(19) United States
(12) Patent Application Publication
(10) Pub. No.: US 2023/0142739 A1
(43) Pub. Date: May 11, 2023

(54) PROTEIN-PROTEIN INTERACTION STABILIZERS

(71) Applicants: The Regents of the University of California, Oakland, CA (US); Eindhoven University of Technology, Eindhoven (NL); Taros Chemicals GmbH & Co. KG, Dortmund (DE)

(72) Inventors: Michelle R. Arkin, San Francisco, CA (US); Lucas Brunsveld, Eindhoven (NL); Christian Ottmann, Eindhoven (NL); Adam R. Renslo, San Francisco, CA (US); R. Jeffrey Neitz, San Francisco, CA (US); Mengqi Zhong, San Francisco, CA (US); Kenneth K. Hallenbeck, San Francisco, CA (US); Priyadarshini Jaishankar, San Francisco, CA (US); Shubhankar Dutta, San Francisco, CA (US); John K. Morrow, San Francisco, CA (US); Eline Sijbesma, Eindhoven (NL); Bente Aminhan Somsen, Eindhoven (NL); Galen Patrick Miley, Eindhoven (NL); Emira Josien Visser, Eindhoven (NL); Peter James Cossar, Eindhoven (NL); Madita Wolter, Eindhoven (NL); Thorsten Genski, Dortmund (DE); Laura Mariana Levy, Dortmund (DE); Torsten Hoffmann, Dortmund (DE); Dimitrios Tzalis, Dortmund (DE); Dario Valenti, Dortmund (DE); Markella Konstantinidou, San Francisco, CA (US)

(21) Appl. No.: 17/915,706

(22) PCT Filed: Apr. 2, 2021
(86) PCT No.: PCT/US2021/025604

§ 371 Date: Sep. 29, 2022

Related U.S. Application Data
(60) Provisional application No. 63/050,045, filed on Jul. 9, 2020.
(60) Provisional application No. 63/004,860, filed on Apr. 3, 2020.

(51) Int. Cl. A61K 31/495; A61K 45/06; C07D 295/185; A61K 31/4453; A61K 31/5375; C07D 265/30; C07D 233/58; C07D 233/90; C07D 233/64; C07D 401/04; A61K 31/4439; A61K 31/4164; C07D 235/24; A61K 31/4184; A61K 31/415; C07D 231/42; C07C 311/16; A61K 31/18; A61K 31/397; A61K 31/40; C07D 207/48; C07D 295/26; A61K 31/551; C07D 211/58; C07D 211/42; C07D 211/44; C07D 217/08; C07D 215/58; C07D 309/14; A61K 31/4468; A61K 31/4462; A61K 31/4465; A61K 31/454; A61K 31/472; A61K 31/47; A61K 31/404; A61K 31/538; A61K 31/498; C07D 209/08; C07D 241/50; C07D 265/36; G01N 33/68; C07D 241/08

(52) U.S. Cl. CPC A61K 31/495; A61K 45/06; C07D 295/185; A61K 31/4453; A61K 31/5375; C07D 265/30; C07D 233/58; C07D 233/90; C07D 233/64; C07D 401/04; A61K 31/4439; A61K 31/4164; C07D 235/24; A61K 31/4184; A61K 31/415; C07D 231/42; C07C 311/16; A61K 31/18; A61K 31/397; A61K 31/40; C07D 207/48; C07D 295/26; A61K 31/551; C07D 211/58; C07D 211/42; C07D 211/44; C07D 217/08; C07D 215/58; C07D 309/14; A61K 31/4468; A61K 31/4462; A61K 31/4465; A61K 31/454; A61K 31/472; A61K 31/47; A61K 31/404; A61K 31/538; A61K 31/498; C07D 209/08; C07D 241/50; C07D 265/36; G01N 33/6845; C07D 241/08

(57) ABSTRACT

Provided herein, inter alia, are stabilizers of protein-protein interactions and methods of identifying and using the same.

### CROSS-REFERENCES TO RELATED APPLICATIONS

This application claims the benefit of U.S. Provisional Application No. 63/004,860, filed Apr. 3, 2020, and U.S. Provisional Application No. 63/050,045, filed Jul. 9, 2020, which are incorporated herein by reference in their entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-672001WO_Sequence_Listing_ST25, created Mar. 31, 2021, 16,358 bytes, machine format IBM-PC, MS Windows operating system, is hereby incorporated by reference.

### BACKGROUND

Once considered ‘undruggable’, protein-protein interactions (PPIs) have been successfully targeted by drug-like molecules in the past 15-20 years (465-468). In contrast to the fruitful development of PPI disruptors, examples of targeted small-molecule PPI stabilizers are relatively scarce, and dedicated screening approaches for PPI stabilizer identification are virtually absent (469-471). Stabilization of PPI allows for diverse functional outcomes, depending of the PPI at hand, and includes inhibition of the transcription process, and inhibition of activity associated with disease progression.

Therapeutic proof-of-concept for PPI stabilization has been provided by natural products, including the anti-tumor drug paclitaxel and immune suppressants rapamycin and FK506 (470, 471). Additionally, a number of successes using synthetic molecules have been reported, such as the BRD4-dimer stabilizer (biBET) (472) and the allosteric stabilizer of the tetramer transthyretin (tafamidis) (473). Synthetic approaches—proteolysis targeting chimeras (PROTACs) and immunomodulatory drugs (iMiDs)—apply this principle to drive the association of two proteins that would not otherwise interact (474). These clinical and chemical-biology applications justify the development of technology platforms to allow systematic stabilization of PPI, especially given the fact that most discoveries of PPI stabilizing molecules have been serendipitous. The design rules for a good stabilizer are poorly understood and technical difficulties complicate assay development. There is thus an unmet need for approaches that overcome inherent limitations of conventional ligand screening to identify PPI stabilizers. Disclosed herein, inter alia, are solutions to these and other problems known in the art.

### BRIEF SUMMARY

In an aspect is provided a compound having the general formula R¹-L¹-W-L³-R³. L¹ and L³ are independently substituted or unsubstituted covalent linkers. R¹ is a 14-3-3 K120 binding moiety. W is a substituted or unsubstituted 14-3-3 binding linker. R³ is a client protein binding moiety.

In an aspect is provided a compound having the general formula R²-L²-W-L³-R³, wherein R² is a 14-3-3 C38 covalent binding moiety. L² is independently a substituted or unsubstituted covalent linker. L³, W, and R³ are as described herein.

In an aspect is provided a compound having the general formula R²-L²-W-L³-R³, wherein R² is a 14-3-3 C38 non-covalent binding moiety. L² is independently a substituted or unsubstituted covalent linker. L³, W, and R³ are as described herein.

In an aspect is provided a pharmaceutical composition including a compound described herein, or a pharmaceutically acceptable salt thereof, and a pharmaceutically acceptable excipient.

In an aspect is provided a method of increasing the level of a 14-3-3 protein-client protein complex in a subject, the method including administering a compound described herein to the subject.

In an aspect is provided a method of increasing the level of a 14-3-3 protein-client protein complex in a cell, the method including contacting the cell with a compound described herein.

In an aspect is provided a method of treating an inflammatory disease, cancer, an autoimmune disease, a neurodegenerative disease, a metabolic disease, or cystic fibrosis in a subject in need thereof, the method including administering to the subject in need thereof an effective amount of a compound described herein.

In an aspect is provided a method of treating a cancer in a subject in need thereof, the method including administering to the subject in need thereof an effective amount of a compound described herein.

In an aspect is provided a method of identifying a chemical compound that modulates the binding of a protein to a client protein, the method including: contacting a first candidate compound with a protein including a solvent exposed reactive amino acid side chain proximal to a client protein binding site, thereby forming a protein conjugate, wherein the first candidate compound includes a first candidate chemical moiety covalently bound to a first reactive group, wherein the first reactive group is specifically reactive with the solvent exposed reactive amino acid side chain, which is not a cysteine side chain; contacting the protein conjugate with the client protein thereby forming a conjugate-client complex; and detecting a change in stability of the conjugate-client complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates binding of a protein to a client protein, the method including: contacting a client protein with a protein including a solvent exposed reactive amino acid side chain proximal to a client protein binding site, thereby forming a protein-client complex; contacting the protein-client complex with a first candidate compound thereby forming a conjugate-client complex, wherein the first candidate compound includes a first candidate chemical moiety covalently bound to a first reactive group, wherein the first reactive group is specifically reactive with the solvent exposed reactive amino acid side chain, which is not a cysteine side chain, and wherein the first candidate compound covalently attaches to the solvent exposed reactive amino acid side chain to form the conjugate-client complex; and detecting a change in stability of the conjugate-client complex relative to the stability of the protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates binding of a protein to a client protein, the method including: contacting a first candidate compound with a client protein including a solvent exposed reactive amino acid side chain, thereby forming a client protein conjugate, wherein the first candidate compound includes a first candidate chemical moiety covalently bound to a first reactive group, wherein the first reactive group is specifically reactive with the solvent exposed reactive amino acid side chain; contacting the client protein conjugate with a protein thereby forming a conjugate-protein complex; and detecting a change in stability of the conjugate-protein complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates binding of a protein to a client protein, the method including: contacting a protein with a client protein including a solvent exposed reactive amino acid side chain thereby forming a protein-client complex; contacting the protein-client complex with a first candidate compound thereby forming a conjugate-protein complex, wherein the first candidate compound includes a first candidate chemical moiety covalently bound to a first reactive group, wherein the first reactive group is specifically reactive with the solvent exposed reactive amino acid side chain, and wherein the first candidate compound covalently attaches to the solvent exposed reactive amino acid side chain to form the conjugate-protein complex; and detecting a change in stability of the conjugate-protein complex relative to the stability of the protein-client complex, wherein the protein-client complex includes the protein and the client protein in the absence of the first candidate compound covalently bound to the solvent exposed reactive amino acid side chain, thereby identifying the first candidate compound as the first chemical compound that modulates binding of the protein to the client protein.

In an aspect is provided a method of treating a disease in a subject in need thereof, the method including administering to the subject an effective amount of a chemical compound that stabilizes binding of a protein to a client protein, wherein the chemical compound is identified by any one of the methods described herein.

### BRIEF DESCRIPTION OF THE DRAWINGS

FIG. 1. Residues to mutate for fragment capture, shown in grey and black. Residues near the identified hot spot (see FIG. 2) are shown in dark grey. Residues already tested are shown in text (C38, N42C, S45C). FIG. 1 shows the residues within 5 Å of the peptide binding groove. They are (14-3-3σ numbering and residue IDs): Black (near hot spot, see FIG. 2): C38, N42, S45, V46, E115, F119, K122, D126, P167, I168, G171, L172, L174, N175, and I219. Grey: E39, R56, R60, Y130, E133, V178, E182, L222, D225, N226, and L229.

FIG. 2. A hotspot (dark grey) identified by fragment-based discovery. Several targets, irrespective of peptide structure, are stabilized by fragments/molecules that bind in this site.

FIG. 3. Numbering for phosphorylated peptide in binding groove (5m36; CDC25C/14-3-3). Note that this structure is turned 180° from FIGS. 1 and 2 (so that the sequence reads N→C). The binding hotspot identified in FIG. 2 is next to M1 and under N4. CDC25C peptide sequence: S₋₉R₋₈S₋₇G₋₆L₋₅Y₋₄R₋₃S₋₂P₋₁pSM₁P₂E₃N₄L₅N₆R₇P₈R₉ (SEQ ID NO:1).

FIG. 4. Primary binding cleft: The natural product fusicoccane-A (FC or FC-A) stabilizes 14-3-3/client complexes. 14-3-3 protein stabilizes diverse peptide conformations.

FIG. 5. Illustration of the approach for selecting stabilizers by disulfide trapping: select for cooperativity. The cysteine-containing protein is incubated with an arrayed disulfide-fragment library under reducing conditions in the apo state (i) or bound to ERα-pp (ii) LC/MS spectra of tethering screen results—illustrates how disulfide libraries are screened.

FIG. 6. Small molecules stabilize 14-3-3σ/Erα-pp binding, for example Frag001 and Frag002.

FIG. 7. Fragments binding to the interface of 14-3-3 with a peptide derived from the p65 subunit of NFκB. Examples from the collection of aldehyde fragments used in the protein crystal based screening.

FIG. 8. Fragments binding to the interface of 14-3-3 with a peptide derived from the p65 subunit of NFκB. Crystal structures of three fragment ‘hits’ covalently bound to Lys122 of 14-3-3σ (ribbons) in the direct vicinity of the NFκB phosphopeptide (compound not enclosed in mesh). The final 2Fo-Fc electron density map is shown as mesh (contoured at 16).

FIGS. 9A-9C. Structure and activity of extended fragments derived from the initial hit TCF521. (FIG. 9A) Crystal structure of extended fragments (top left compound) binding to the complex of 14-3-3σ (ribbons and protein backbone) and a peptide derived from NFκBp65 (peptide on right). The final 2Fo-Fc electron density for the fragment is shown as mesh (contoured at 1). (FIG. 9B) Details of the interaction of the fragments with 14-3-3σ and the NFκB peptide. Residues from 14-3-3σ important for binding the fragments are shown as sticks, with hydrophobic interactions visualized by semi-transparent van-der-Waals surfaces and polar contacts depicted as dotted lines. Water molecules involved in these interactions are shown as spheres. (FIG. 9C) FP measurement of binding of a FITC-labelled NFκB peptide binding to 14-3-3σ in the presence of increasing concentrations of the fragments.

FIG. 10. Principal of optimizing orthosteric PPI stabilization. Increasing the interaction with the protein partner that contributed less to the composite binding pocket of the stabilizer (NFκB, grey surface) results in increased stabilization, whereas further enhancing the interaction with the dominant partner protein (14-3-3, white surface) does not contribute to the stabilizing effect.

FIG. 11. X-ray crystal structures of fragments 1-5 in complex with 14-3-3σ(C42) (white surface; C42) and ERα-pp (right sticks).

FIGS. 12A-12D. Selectivity of hit fragment 2. FIG. 12A) Dose-response curves obtained by MS, analyzing % tethering for titrations of 2 to 14-3-3σ apo (− peptide; circle symbol) or bound to different interaction partner-derived peptide motifs; ERα-pp (square symbol), TASK3-pp (triangle symbol), ExoS (inverted triangle symbol) or TAZ-pp (diamond symbol), starting from 1 mM. FIGS. 12B-12D) Overlays of crystal structures of 14-3-3σ (white surface) bound by 2, and TASK3-pp (PDB: 3P1N) (FIG. 12B), ExoS (PDB: 2002) (FIG. 12C), or TAZ-pp (PDB: 5N75) (FIG. 12D) illustrating (in)compatibility of binding surface areas. Fragment (dark gray) and peptides (medium gray) in space-filling representation. Fluorescence anisotropy data (mean+SD; triplicates) and non-linear fit for titration of 2 (square symbol) to 14-3-3σ. FC-A (inverted triangle symbol) and DMSO (diamond symbol) are included as controls. Sequences shown: KRRKpS³⁷³V-OH (SEQ ID NO:55, FIG. 12B); GLLDALDLAS (SEQ ID NO:56, FIG. 12C); RSHpS⁸⁹SPASLQ (SEQ ID NO:57, FIG. 12D).

FIG. 13. Sequence and conformational diversity of 14-3-3 ligands.

FIGS. 14A-14B. Kinetic effects of disulfide conjugation and stabilization of 14-3-3σ (C42)/ERα-pp observed in titration curves of disulfide-fragment hits. Fluorescence anisotropy (r) plotted versus protein (FIG. 14A) or compound (FIG. 14B) concentration measured directly (t0) and after overnight incubation at RT. At saturating concentration, disulfide-fragment conjugation to the protein is instantaneous as observed from titrations of 14-3-3σ (C42) to 100 nM fluorescein-ERα-pp and 100 μM of FC-A (inverted triangle symbol); DMSO (diamond symbol); and 1 (square symbol) (FIG. 14A). No difference was observed in stabilization for ERα-pp binding affinity over time. Right: Kinetics of protein-peptide stabilization are dependent on disulfide-fragment concentration, observed from increased anisotropy values at intermediate concentrations (0.1-10 μM), resulting in a EC₅₀ shift (indicated by arrows) for disulfide-fragment titrations to a mixture of 1 μM 14-3-3σ(C42) and 100 nM fluorescein-ERα-pp, which was not observed for FC-A. Dashed lines indicate assay window based on protein and disulfide-fragment concentrations.

FIGS. 15A-15C. Titration curves of disulfide hits for 14-3-3σ(C42). Fluorescence anisotropy (r) plotted versus compound concentration. FIG. 15A) Left: Titrations of 1 (square symbol), 2 (circle symbol), a second lot of 2 (2*triangle symbol), FC-A (inverted triangle symbol) and DMSO (diamond symbol) to a mixture of 1 tM 14-3-3σ(C42) and 100 nM fluorescein-ERα-pp. These data are independent replicates of data shown in main text (FIG. 15B). Right: Titrations of 3 (light diamond symbol), 4 (medium diamond symbol) and 5 (dark diamond symbol) added to a mixture of 1 μM 14-3-3σ(C42) and 100 nM fluorescein-ERα-pp, FIGS. 15B-15C) Titration data for additional disulfide-fragments selected for follow-up from tethering screen, as cooperative, neutral (FIG. 15B), or competitive hits (FIG. 15C). EC₅₀ and IC₅₀ values shown in the tables (right of the graphs). EC₅₀ values are reported at the inflection point for the curves.

FIGS. 16A-16B. Concept of imine tethering. FIG. 16A: Lysine residues can be targeted with aldehydes forming an aldimine bond. FIG. 16B: Lysine 122 of 14-3-3 is located in a deep composite binding pocket created by the NF-κB/14-3-3 complex (surface representation of 14-3-3 in white and the p65 subunit of NF-κB in grey). Sequence shown: IPGRRS (SEQ ID NO:8).

FIG. 17. Chemical structures of initial fragments screened.

FIG. 18. Extended aldehyde fragment library to investigate the contribution of an activation of the aldehyde. Fragments TCF521-011, TCF521-025, TCF521-027, TCF521-028, TCT521-033, and TCF521-037 were detected in the electron density map of soaked p65/14-3-3 crystals. Fragment TCF521-021 induced crystal cracking, hence prevented data collection in the assay tested.

FIGS. 19A-19E. Disulfide trapping identify ligands for 14-3-3σ. FIG. 19A: Target pocket for the site-directed disulfide-trapping approach, highlighting two cysteine mutations (C42, C45; grey surface areas) in the 14-3-3σ (white surface)/ERα-pp (grey spheres) pocket. FIG. 19B: Chemical structures of previously described 14-3-3σ/ERα-pp stabilizers 1 and 2. FIGS. 19C-19E: Chemical structures and disulfide trapping screening results for C45 hits. Mass spectrometry spectra for 14-3-3σ(C45) conjugated to fragment 3 (FIG. 19C), 4 (FIG. 19D), and 5 (FIG. 19E) in the absence (left) or presence (right) of ERα-pp. The adduct shift between apo protein [expected mass 26,536 Da, 2-mercaptoethanol (βME)-capped mass 26,612 Da] and protein-disulfide conjugate mass is indicated with arrows. Conditions for mass spectrometry: 100 nM 14-3-3σ, 200 nM ERα-pp, 100 μM fragment, 1 mM βME.

FIG. 20. Close-up view of the binding pocket for co-crystal structures of 14-3-3σ(C45)-tethered fragment 3 (sticks). 2Fₒ-F_{c} electron density maps are contoured at 16, 14-3-3σ is shown as white surface, ERα-pp as dark sticks.

FIGS. 21A-21B. Chemical structures of aldehydes tested in imine tethering screen described in Example 9.

FIGS. 22A-22F. Investigation of 13 representative 14-3-3/peptide interactions reveals selective stabilization of the 14-3-3/Pin1_72 complex by 28. FIG. 22A: Radar plot of the SFs determined by FA protein titrations in presence of 100 μM fragment. Fragment 28 shows preferential binding for the Pin1_72/14-3-3γ comparable to the effect of FCA on the ERα/14-3-3γ interaction. Right: close-up. FIG. 22B: The SF values determined with 14-3-3γ titrations in presence of 100 μM 13, 27 or 28 in FA assays (n=2). FIG. 22C: Structural overlay of the known 14-3-3 binding epitopes used in this study. FIG. 22D: Overlay of the binding pose of 13, 27 and 28 (line representation) with the AS160 binding epitope (PDB: 7NIX). FIG. 22E: Overlay of crystal structures of 13 (2Fo-Fc map at 1 as mesh) binding to the p65_45 (violet sticks, carton)/14-3-3γ complex (PDB: 7NQP) and the CFTR (cyan sticks, cartoon)/14-3-3 complex (PDB: 5D3F, FC-A hidden for clarity). Hydrophobic contacts between 13 and Ile+1 of p65 and Val+1 of CFTR are indicated by transparent spheres. FIG. 22F: Cooperative analysis of ternary complex formation using 28 with Pin1 and p65 peptides shows that stabilization of the ternary complex is driven by the unique environment created by the partner peptide binding.

FIGS. 23A-23C. Synthesis of focused fragment libraries 1 (FIG. 23A), 2 (FIG. 23B), and 3 (FIG. 23C) described in Example 11.

FIGS. 24A-24B. Synthesis of focused fragment libraries 4 (FIG. 24A) and 5 (FIG. 24B) described in Example 11.

FIGS. 25A-25F. Optimization of 23z. FIG. 25A: Ternary structure of 23z (light spheres) in complex with 14-3-3σΔC (white surface) and p65_45 (grey cartoon, transparent spheres) (PDB: 7NJ9). Carbons of the bicyclic head group are numbered. FIG. 25B: Ternary structure of 24b in complex with p65_45/14-3-3σΔC (PDB: 7BIQ). Distance between the 2-methyl and water (grey spheres) are indicated with black dashes. Polar contacts are shown as black dashed lines. FIG. 25C: Structure of 24e binding to the p65_45/14-3-3σΔC complex (as in FIG. 25A). Hydrophobic contacts are indicated with transparent spheres. FIG. 25D: FA compound titrations with 50 μM 14-3-3γ and 100 nM p65_45. FIG. 25E: FA protein titrations with 1 mM fragment and 100 nM p65_45. FIG. 25F: Structure of 24j binding to the p65_45/14-3-3σΔC complex (PDB: 7BIW)). A beneficial hydrogen bond is formed between the backbone carbonyl of p65s′ Pro47 and 24j (black dash).

FIG. 26. Selected chloroacetamide compounds.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings. In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

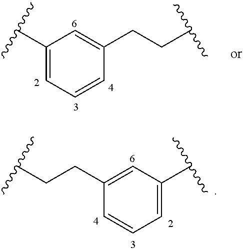 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted (e.g., C₁-C₈, C₁-C₆ or C₁-C₄) alkyl, substituted or unsubstituted (e.g., 2 to 8 membered, 2 to 6 membered, or 2 to 4 membered) heteroalkyl, substituted or unsubstituted (e.g., C₂-C₆ or C₂-C₄) alkenyl, substituted or unsubstituted (e.g., 2 to 6 membered or 2 to 4 membered) heteroalkenyl, substituted or unsubstituted (e.g., C₂-C₆ or C₂-C₄) alkynyl, substituted or unsubstituted (e.g., 2 to 6 membered or 2 to 4 membered) heteroalkynyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀ alkyl, C₁-C₁₂ alkyl, C₁-C₈ alkyl, C₁-C₆ alkyl, C₁-C₄ alkyl, or C₁-C₂alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth herein, for example in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on a R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R¹⁰⁰. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g. R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g. R^{1.3}, R^{2.3}, R^{3.3}, R^{4.3}, R^{5.3} . . . R^{100.3}. R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B. etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g. when R^{WW.1} is R^{WW.2} substituted alkyl, examples of groups so formed include but are not limited to itself optionally substituted by 1 or more R^{WW.2}, which R^{WW.2} is optionally substituted by one or more R^{WW.3}. By way of example when R^{WW.1} is alkyl, groups that could be formed, include but are not limited to:

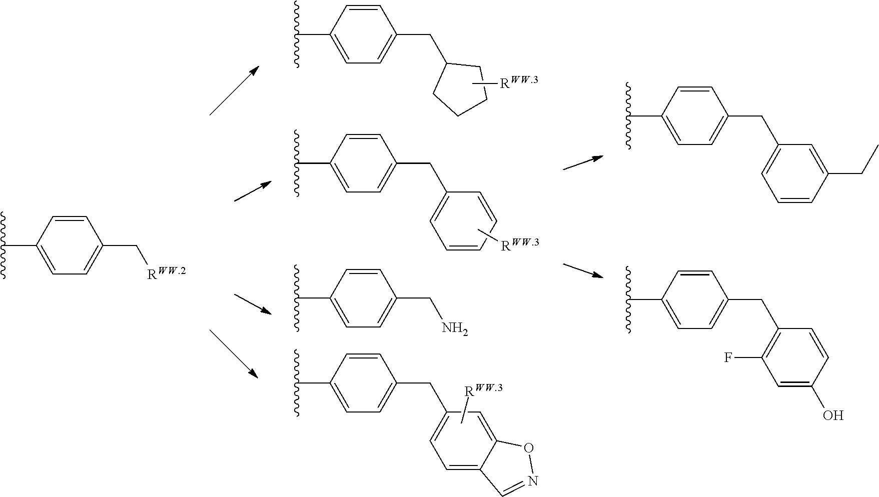

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N3, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N3, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N3, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g. 1, 2, 3, 1A, 2A, 3A, 1B, 2B, 3B. etc.). R^{WW.1}, R^{WW.2}, and R^{WW.3}, are as defined above.

In the event that any L linker group recited in a claim or chemical formula description set forth herein (i.e. an L^{WW} substituent) is not explicitly defined, then that L group (L^{WW} group) is herein defined as independently —O—, —NH—, —C(O)—, —C(O)NH—, —NHC(O)—, —NHC(O)NH—, —C(O)O—, —OC(O)—, —S—, —SO₂NH—, —NHSO₂—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3}, are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein may be bound, for example, by covalent bond, linker (e.g. a first linker of second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g., phosphines) to form, for example, phosphate diester bonds;
(n) azides coupled to alkynes using copper catalyzed cycloaddition click chemistry; and
(o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

The term “electrophilic” as used herein refers to a chemical group that is capable of accepting electron density. An “electrophilic substituent,” “electrophilic chemical moiety,” or “electrophic moiety” refers to an electron-poor chemical group, substitutent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier” or “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

The term “covalent cysteine modifier moiety” as used herein refers to a monovalent electrophilic moiety that is able to measurably bind to a cysteine amino acid. In embodiments, the covalent cysteine modifier moiety binds via an irreversible covalent bond. In embodiments, the covalent cysteine modifier moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

“Nucleophilic” as used herein refers to a chemical group that is capable of donating electron density.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “client protein” as used herein refers to a protein that is capable of binding to another protein (e.g., a 14-3-3 protein). In embodiments, the client protein interaction with the other protein is stabilized with chemical compound as set forth herein. In embodiments the client protein is a 14-3-3 client protein, which is a client protein of a 14-3-3 protein.

The term “14-3-3 protein” as used herein refers to a protein (or portion thereof) that is a member of the 14-3-3 protein family, including, but not limited to, the various human isoforms (β, γ, ε, ζ, η, τ/θ and σ). When specified, the term can refer to a specific isoform or group of isoforms. In one embodiment, the term refers to the σ isoform. In embodiments, the 14-3-3 proteins influence the function of bound phosphoserine and/or threonine phosphorylated proteins via a variety of mechanisms including sequestering them from cellular targets, controlling their enzymatic activity, relocating them or acting as adaptor molecules in mediating the association of two distinct client proteins. Thus, in embodiments, 14-3-3 proteins regulate pathways involved in growth factor signaling and cell cycle progression. The 14-3-3 protein may interact with more than 300 different partners (client proteins), including Raf kinases, heat shock proteins, oncogenes, and tumor suppressors. 14-3-3 proteins are central regulators in many biological processes and pathologies. In embodiments, 14-3-3 binding antagonizes multiple transcription factors that act as oncogenic drivers. In embodiments, 14-3-3 protein binds to an ERrα protein, phosphorylated at the T594 residue, and reduces the transcriptional activity of ERrα. In embodiments, the 14-3-3 protein is 14-3-3σ (14-3-3sigma) (e.g., Entrez 2810, UniProt P31947, RefSeq NP_006133). In embodiments, the 14-3-3 protein is 14-3-3β (14-3-3beta) (e.g., Entrez 7529, UniProt P31946, Q4VY19, RefSeq NP_003395). In embodiments, the 14-3-3 protein is 14-3-3ε (14-3-3epsilon) (e.g., Entrez 7531, UniProt P62258, RefSeq NP_006752). In embodiments, the 14-3 protein is 14-3-3η (14-3-3eta) (e.g., Entrez 7533, UniProt Q04917, RefSeq NP_003396). In embodiments, the 14-3-3 protein is 14-3-3γ(14-3-3gamma) (e.g., Entrez 7532, UniProt P61981, RefSeq NP_36611). In embodiments, the 1433 protein is 14-3-3τ (14-3-3tau) (e.g., Entrez 10971, UniProt P27348, RefSeq NP_006817). In embodiments, the 14-3-3 protein is 14-3-3ζ (14-3-3zeta) (e.g., Entrez 7534, UniProt P63104, RefSeq NP_003397).

In embodiments, the 14-3-3 protein is phosphorylated. In embodiments, the 14-3-3 client is a phosphoserine protein. In embodiments, the 14-3-3 client is a phosphothreonine protein. In embodiments, the 14-3-3 client is a phosphorylated peptide (a phosphopeptide) derived from the 14-3-3 client protein. In embodiments, the 14-3-3 client is a phosphorylated peptide (phosphopeptide) representing the 14-3-3 protein binding motif of the client protein.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer associated with 14-3-3 protein function, 14-3-3 protein associated cancer, 14-3-3 protein associated disease (e.g., cancer)) means that the disease (e.g., cancer) is caused by (in whole or in part), or a symptom of the disease is caused by (in whole or in part) the substance or substance activity or function. For example, a cancer associated with 14-3-3 protein activity or function may be a cancer that results (entirely or partially) from aberrant 14-3-3 protein function (e.g., protein-protein interaction, signaling pathway) or a cancer wherein a particular symptom of the disease is caused (entirely or partially) by aberrant 14-3-3 protein activity or function. As used herein, what is described as being associated with a disease, if a causative agent, could be a target for treatment of the disease. For example, a cancer associated with 14-3-3 protein function or a 14-3-3 protein associated disease (e.g., cancer), may be treated with a 14-3-3 protein modulator or 14-3-3 protein inhibitor, in the instance where increased 14-3-3 protein function (e.g., signaling pathway activity) causes the disease (e.g., cancer). For example, a cancer associated with 14-3-3 protein function or a 14-3-3 protein associated disease (e.g., cancer), may be treated with a 14-3-3 protein modulator or 14-3-3 protein inhibitor, in the instance where increased 14-3-3 protein function (e.g. signaling pathway activity) causes the disease (e.g., cancer). A cancer associated with 14-3-3 protein function or a 14-3-3 protein associated disease (e.g., cancer), may be treated with a 14-3-3 protein modulator or 14-3-3 protein activator, in the instance where decreased 14-3-3 protein function (e.g., signaling pathway activity) causes the disease (e.g., cancer).

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. In embodiments, an anti-cancer agent is an inhibitor of K-Ras, RAF, MEK, Erk, PI3K, Akt, RTK, or mTOR. In embodiments, an anti-cancer agent is an MDM2 inhibitor or a genotoxic anti-cancer agent. In embodiments, an anti-cancer agent is nutlin-1, nutlin-2, nutlin-3, nutlin-3a, nutlin-3b, YH239-EE, MI-219, MI-773, MI-77301, MI-888, MX69, RG7112, RG7388, RITA, idasanutlin, DS-3032b, or AMG232. In embodiments, an anti-cancer agent is an alkylating agent, intercalating agent, or DNA replication inhibitor. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e. paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

“Chemotherapeutic” or “chemotherapeutic agent” is used in accordance with its plain ordinary meaning and refers to a chemical composition or compound having antineoplastic properties or the ability to inhibit the growth or proliferation of cells.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components. For example, binding of a 14-3-3 protein with a compound as described herein may increase the interactions between the 14-3-3 protein and downstream effectors or signaling pathway components, resulting in changes in cell growth, proliferation, or survival.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g., humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, acute lymphoblastic leukemia (ALL), chronic lymphocytic leukemia (CLL), acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myeloid leukemia (AML), chronic myeloid leukemia (CML), leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myelodysplastic syndrome (MDS), myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma (MCL), follicular lymphoma, marginal zone B-cell lymphoma (MZL), mucosa-associated lymphatic tissue lymphoma (MALT), extranodal lymphoma, nodal (monocytoid B-cell) lymphoma, splenic lymphoma, diffuse large cell B-lymphoma (DLBCL), activated B-cell subtype diffuse large B-cell lymphoma (ABC-DBLCL), germinal center B-cell like diffuse large B-cell lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungocides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The term “infection” or “infectious disease” refers to a disease or condition that can be caused by organisms such as a bacterium, virus, parasite, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the disease is Bacterial vaginosis (i.e., bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e., a bacterial inflammation of the meninges); Bacterial pneumonia (i.e., a bacterial infection of the lungs); Urinary tract infection; Bacterial gastroenteritis; or Bacterial skin infections (e.g., impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

As used herein, the term “metabolic disease” or “metabolic disorder” refers to a disease or condition in which a subject's metabolism or metabolic system (e.g., function of storing or utilizing energy) becomes impaired. Examples of metabolic diseases that may be treated with a compound, pharmaceutical composition, or method described herein include diabetes (e.g., type I or type II), obesity, metabolic syndrome, or a mitochondrial disease (e.g., dysfunction of mitochondria or aberrant mitochondrial function).

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein may include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

The term “prevent” refers to a decrease in the occurrence of 14-3-3 protein associated disease symptoms or 14-3-3 protein associated disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity increasing amount,” as used herein, refers to an amount of agonist required to increase the activity of a 14-3-3 protein relative to the absence of the agonist. A “function increasing amount,” as used herein, refers to the amount of agonist required to increase the function of a 14-3-3 protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

An amino acid residue in a protein “corresponds” to a given residue when it occupies the same essential structural position within the protein as the given residue. For example, a selected residue in a selected protein corresponds to C38 of human 14-3-3σ protein when the selected residue occupies the same essential spatial or other structural relationship as C38 in human 14-3-3σ protein. In some embodiments, where a selected protein is aligned for maximum homology with the human 14-3-3σ protein, the position in the aligned selected protein aligning with C38 is said to correspond to C38. Instead of a primary sequence alignment, a three dimensional structural alignment can also be used, e.g., where the structure of the selected protein is aligned for maximum correspondence with the human 14-3-3σ protein and the overall structures compared. In this case, an amino acid that occupies the same essential position as C38 in the structural model is said to correspond to the C38 residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

The term “chemical compound” is used in accordance with its plain ordinary meaning and refers to a chemical substance composed of many identical molecules composed of atoms from more than one element held together by chemical bonds.

The term “chemical moiety” refers to a part of a molecule responsible for characteristic chemical reactions of that molecule. In embodiments, “chemical moiety” refers to a functional group. In embodiments, “chemical moiety” refers to several functional groups.

The term “14-3-3 K120 binding moiety” refers to a moiety of a compound capable of contacting or binding to an amino acid in a 14-3-3 protein corresponding to K120 of 14-3-3R (14-3-3tau). The term “14-3-3 K120 non-covalent binding moiety” refers to a moiety of a compound capable of non-covalently binding to an amino acid in a 14-3-3 protein corresponding to K120 of 14-3-3τ (14-3-3tau). The term “14-3-3 K120 covalent binding moiety” refers to a moiety of a compound capable of covalently binding to an amino acid in a 14-3-3 protein corresponding to K120 of 14-3-3τ (14-3-3tau). In embodiments, a 14-3-3 K120 binding moiety is a 14-3-3β K122 binding moiety (14-3-3beta). In embodiments, a 14-3-3 K120 binding moiety is a 14-3-3ε K123 binding moiety (14-3-3epsilon). In embodiments, a 14-3-3 K120 binding moiety is a 14-3-3η K125 binding moiety (14-3-3eta). In embodiments, a 14-3-3 K120 binding moiety is a 14-3-3γ K125 binding moiety (14-3-3gamma). In embodiments, a 14-3-3 K120 binding moiety is a 14-3-3σ K122 binding moiety (14-3-3sigma). In embodiments, a 14-3-3 K120 binding moiety is a 14-3-3τ K120 binding moiety (14-3-3tau). In embodiments, a 14-3-3 K120 binding moiety is a 14-3-3ζ K120 binding moiety (14-3-3zeta).

The term “14-3-3 binding linker” refers to a divalent chemical linker capable of binding or contacting a 14-3-3 protein.

The term “client protein binding moiety” refers to a moiety of a compound capable of contacting or binding to a client protein of a 14-3-3 protein.

The term “14-3-3 C38 binding moiety” refers to a moiety of a compound capable of contacting or binding to an amino acid in a 14-3-3 protein corresponding to C38 of 14-3-3σ (14-3-3sigma). The term “14-3-3 C38 non-covalent binding moiety” refers to a moiety of a compound capable of non-covalently binding to an amino acid in a 14-3-3 protein corresponding to C38 of 14-3-3σ (14-3-3sigma). The term “14-3-3 C38 covalent binding moiety” refers to a moiety of a compound capable of covalently binding to an amino acid in a 14-3-3 protein corresponding to C38 of 14-3-3σ (14-3-3sigma). In embodiments, a 14-3-3 C38 binding moiety is a 14-3-3β N40 binding moiety (14-3-3beta). In embodiments, a 14-3-3 C38 binding moiety is a 14-3-3ε V39 binding moiety (14-3-3epsilon). In embodiments, a 14-3-3 C38 binding moiety is a 14-3-3η N39 binding moiety (14-3-3eta). In embodiments, a 14-3-3 C38 binding moiety is a 14-3-3γ N39 binding moiety (14-3-3gamma). In embodiments, a 14-3-3 C38 binding moiety is a 14-3-3σ C38 binding moiety (14-3-3sigma). In embodiments, a 14-3-3 C38 binding moiety is a 14-3-3τ N38 binding moiety (14-3-3tau). In embodiments, a 14-3-3 C38 binding moiety is a 14-3-3ζ N38 binding moiety (14-3-3zeta).

The term “14-3-3 D215 binding moiety” refers to a moiety of a compound capable of contacting or binding to an amino acid in a 14-3-3 protein corresponding to D215 of 14-3-3σ (14-3-3sigma). The term “14-3-3 D215 non-covalent binding moiety” refers to a moiety of a compound capable of non-covalently binding to an amino acid in a 14-3-3 protein corresponding to D215 of 14-3-3σ (14-3-3sigma). The term “14-3-3 D215 covalent binding moiety” refers to a moiety of a compound capable of covalently binding to an amino acid in a 14-3-3 protein corresponding to D215 of 14-3-3σ (14-3-3sigma). In embodiments, a 14-3-3 D215 binding moiety is a 14-3-3β D215 binding moiety (14-3-3beta). In embodiments, a 14-3-3 D215 binding moiety is a 14-3-3ε D216 binding moiety (14-3-3epsilon). In embodiments, a 14-3-3 D215 binding moiety is a 14-3-3η D218 binding moiety (14-3-3eta). In embodiments, a 14-3-3 D215 binding moiety is a 14-3-3γ D218 binding moiety (14-3-3gamma). In embodiments, a 14-3-3 D215 binding moiety is a 14-3-3σ D215 binding moiety (14-3-3sigma). In embodiments, a 14-3-3 D215 binding moiety is a 14-3-3τ D213 binding moiety (14-3-3tau). In embodiments, a 14-3-3 D215 binding moiety is a 14-3-3ζ D213 binding moiety (14-3-3zeta).

“ERRγ” refers to a nuclear receptor that in humans is encoded by the ESRRG (EStrogen Related Receptor Gamma) gene. A nuclear receptor is a protein found within cells responsible for sensing steroid and thyroid hormones and certain other molecules. In response, these receptors work with other proteins to regulate the expression of specific genes thereby controlling the development, homeostasis and metabolism of the organism. This receptor is classified as transcription factor. A transcription factor (TF) is a protein that controls the rate of transcription of genetic information from DNA to messenger RNA, by binding to a specific DNA sequence. The function of a transcription factor is to regulate—turn on and off—genes in order to make sure they are expressed in the right cell at the right time and in the right amount throughout the life of the cell and the organism.

“Rel A” refers to a Transcription factor p65 also known as nuclear factor NF-kappa-B p65 subunit. It is a protein that in humans is encoded by the RELA gene. Rel A, also known as p65, is a Rel-associated protein involved in NF-κB heterodimer formation, nuclear translocation and activation. NF-κB is an essential transcription factor complex involved in all types of cellular processes, including cellular metabolism, chemotaxis, etc. Phosphorylation and acetylation of Rel A are crucial post-translational modifications required for NF-κB activation. Rel A has also been shown to modulate immune responses, and activation of Rel A is positively associated with multiple types of cancer.

The term “Estrogen receptor alpha”, “ERα”, or “NR3A1” refers to a hormone receptor activated by estrogen. The term includes any recombinant or naturally-occurring form of ERα, including variants thereof that maintain ERα function or activity (e.g., within at least 30%, 40%, 50%, 60%, 70%, 80%, 90%, 95%, or 100% function or activity compared to wildtype). In embodiments, ERα is encoded by the NR3A1 gene. In embodiments, ERα, has the amino acid sequence set forth in or corresponding to Entrez 2099, UniProt P03372, RefSeq (protein) NP_000116. In embodiments, ERα has the amino acid sequence set forth in or corresponding to RefSeq (protein) NP_000116.2.

The term “Rel A”, “NFκBp65”, or “p65” refers to the NFκB p65 subunit associated with NFκB heterodimer formation, nuclear translocation, and activation. The term includes any recombinant or naturally-occurring form of Rel A, including variants thereof that maintain Rel A function or activity (e.g., within at least 30%, 40%, 50%, 60%, 70%, 80%, 90%, 95%, or 100% function or activity compared to wildtype). In embodiments, Rel A is encoded by the RELA gene. In embodiments, Rel A has the amino acid sequence set forth in or corresponding to Entrez 5970, UniProt Q04206, RefSeq (protein) NP_068810. In embodiments, Rel A has the amino acid sequence set forth in or corresponding to RefSeq (protein) NP_068810.3.

The term “NFκB” or “nuclear factor kappa-light-chain-enhancer of activated B cells” refers to a protein complex associated with transcription of DNA, cytokine production, and cell survival. Incorrect regulation of NFκB may be associated with cancer, inflammatory disease, autoimmune disease, septic shock, infectious diseases, or immune diseases. The term includes any recombinant or naturally-occurring form of NFκB, including variants thereof that maintain NFκB function or activity (e.g., within at least 30%, 40%, 50%, 60%, 70%, 80%, 90%, 95%, or 100% function or activity compared to wildtype). In embodiments, NFκB is NFκBI (e.g., UniProt P19838), NFκB2 (e.g., UniProt Q00653), Rel A (p65), Rel B (e.g., UniProt Q01201), or Rel (c-Rel) (e.g., UniProt Q04864).

The term “serine/threonine-protein kinase B-Raf”, “BRAF”, or “B-RAF” refers to the protein responsible for regulating the MAP kinase/ERKs signaling pathway. The term includes any recombinant or naturally-occurring form of BRAF, including variants thereof that maintain BRAF function or activity (e.g., within at least 30%, 40%, 50%, 60%, 70%, 80%, 90%, 95%, or 100% function or activity compared to wildtype). In embodiments, BRAF is encoded by the BRAF gene. In embodiments, BRAF has the amino acid sequence set forth in or corresponding to Entrez 673, UniProt P15056, RefSeq (protein) NP_004324. In embodiments, BRAF has the amino acid sequence set forth in or corresponding to RefSeq (protein) NP_004324.2.

The term “RAF proto-oncogene serine/threonine-protein kinase”, “C-RAF”, “CRAF” or “Raf-1” refers to the protein that is part of the ERK1/2 pathway and is a MAP kinase. The term includes any recombinant or naturally-occurring form of CRAF, including variants thereof that maintain CRAF function or activity (e.g., within at least 30%, 40%, 50%, 60%, 70%, 80%, 90%, 95%, or 100% function or activity compared to wildtype). In embodiments, CRAF is encoded by the RAF1 gene. In embodiments, CRAF has the amino acid sequence set forth in or corresponding to Entrez 5894, UniProt P04049, RefSeq (protein) NP_002871. In embodiments, CRAF has the amino acid sequence set forth in or corresponding to RefSeq (protein) NP_001341618. In embodiments, CRAF has the amino acid sequence set forth in or corresponding to RefSeq (protein) NP_002871.1.

The term “Son of sevenless homolog”, “SOS”, or “SOS1” refers to the guanine nucleotide exchange factor that interacts with RAS proteins. The term includes any recombinant or naturally-occurring form of SOS1, including variants thereof that maintain SOS1 function or activity (e.g., within at least 30%, 40%, 50%, 60%, 70%, 80%, 90%, 95%, or 100% function or activity compared to wildtype). In embodiments, SOS1 is encoded by the SOS1 gene. In embodiments, SOS1 has the amino acid sequence set forth in or corresponding to Entrez 6654, UniProt Q07889, RefSeq (protein) NP_005624. In embodiments, SOS1 has the amino acid sequence set forth in or corresponding to RefSeq (protein) NP_005624.2.

The term “Estrogen-related receptor gamma”, “ERR-gamma”, “NR3B3”, or “ERRγ” refers to a hormone receptor. The term includes any recombinant or naturally-occurring form of ERRγ, including variants thereof that maintain ERRγ function or activity (e.g., within at least 30%, 40%, 50%, 60%, 70%, 80%, 90%, 95%, or 100% function or activity compared to wildtype). In embodiments, ERRγ is encoded by the ESRRG gene. In embodiments, ERRγ has the amino acid sequence set forth in or corresponding to Entrez 2104 or UniProt P62508.

The term “ubiquitin carboxyl-terminal hydrolase 8” or “USP8” refers to a ubiquitin-specific processing protein. The term includes any recombinant or naturally-occurring form of USP8, including variants thereof that maintain USP8 function or activity (e.g., within at least 30%, 40%, 50%, 60%, 70%, 80%, 90%, 95%, or 100% function or activity compared to wildtype). In embodiments, USP8 is encoded by the USP8 gene. In embodiments, USP8 has the amino acid sequence set forth in or corresponding to Entrez 9101, UniProt P40818, RefSeq (protein) NP_005145. In embodiments, USP8 has the amino acid sequence set forth in or corresponding to RefSeq (protein) NP_005145.3.

### II. Compounds

In an aspect is provided a compound having the general formula R¹-L¹-W-L³-R³. L¹ and L³ are independently substituted or unsubstituted covalent linkers. R¹ is a 14-3-3 K120 binding moiety. W is a substituted or unsubstituted 14-3-3 binding linker. R³ is a client protein binding moiety.

In embodiments, wherein the compound has the general formula R¹-L¹-W-L³-R³, the compound further includes R². In embodiments, wherein the compound has the general formula R¹-L¹-W-L³-R³, the compound further includes -L²-R².

R² is a 14-3-3 C38 non-covalent binding moiety or a 14-3-3 C38 covalent binding moiety. L² is independently a substituted or unsubstituted covalent linker.

In an aspect is provided a compound having the general formula R²-L²-W-L³-R³, wherein R² is a 14-3-3 C38 covalent binding moiety. L² is independently a substituted or unsubstituted covalent linker. L³, W, and R³ are as described herein.

In an aspect is provided a compound having the general formula R²-L²-W-L³-R³, wherein R² is a 14-3-3 C38 non-covalent binding moiety. L² is independently a substituted or unsubstituted covalent linker. L³, W, and R³ are as described herein.

In embodiments, wherein the compound has the general formula R²-L²-W-L³-R³, the compound further includes R¹. In embodiments, wherein the compound has the general formula R²-L²-W-L³-R³, the compound further includes -L¹-R¹. In embodiments, wherein the compound has the general formula R²-L²-W-L³-R³, W is substituted with -L¹-R¹.

In an aspect is provided a compound having the formula R¹-L¹-W-L³-R³. L¹ and L³ are independently substituted or unsubstituted covalent linkers. R¹ is a 14-3-3 K120 binding moiety. W is a substituted or unsubstituted 14-3-3 binding linker. R³ is a client protein binding moiety.

In embodiments, wherein the compound has the formula R¹-L¹-W-L³-R³, the compound further includes R². In embodiments, wherein the compound has the formula R¹-L¹-W-L³-R³, the compound further includes -L²-R².

In embodiments, wherein the compound has the formula R¹-L¹-W-L³-R³, W is substituted with -L²-R².

In an aspect is provided a compound having the formula R²-L²-W-L³-R³, wherein R² is a 14-3-3 C38 non-covalent binding moiety. L² is independently a substituted or unsubstituted covalent linker. L³, W, and R³ are as described herein.

In embodiments, wherein the compound has the formula R²-L²-W-L³-R³, the compound further includes R¹. In embodiments, wherein the compound has the formula R²-L²-W-L³-R³, the compound further includes -L¹-R¹.

In embodiments, wherein the compound has the formula R²-L²-W-L³-R³, W is substituted with -L¹-R¹.

In embodiments, W is substituted with -L⁵-R⁵.

L⁵ is a substituted or unsubstituted covalent linker. R⁵ is a 14-3-3 D215 binding moiety.

In embodiments, W is W¹—W²—W³—W⁴—W⁵—W⁶.

W¹, W², W³, W⁴, W⁵, and W⁶ are independently a bond, —S(O)₂—, —S(O)₃—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenyl), or substituted or unsubstituted heteroarylene (e.g., 5 to 12 membered, 5 to 10 membered, 5 to 9 membered, or 5 to 6 membered).

In embodiments, W is a bond, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenyl), or substituted or unsubstituted heteroarylene (e.g., 5 to 12 membered, 5 to 10 membered, 5 to 9 membered, or 5 to 6 membered).

In embodiments, a substituted W (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W is substituted with a plurality of groups selected from substituent groups, size-limited substituent groups, and lower substituent groups; each substituent group, size-limited substituent group, and/or lower substituent group may optionally be different. In embodiments, when W is substituted, it is substituted with at least one substituent group. In embodiments, when W is substituted, it is substituted with at least one size-limited substituent group. In embodiments, when W is substituted, it is substituted with at least one lower substituent group.

In embodiments, a substituted W¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W¹ is substituted with a plurality of groups selected from substituent groups, size-limited substituent groups, and lower substituent groups; each substituent group, size-limited substituent group, and/or lower substituent group may optionally be different. In embodiments, when W¹ is substituted, it is substituted with at least one substituent group. In embodiments, when W¹ is substituted, it is substituted with at least one size-limited substituent group. In embodiments, when W¹ is substituted, it is substituted with at least one lower substituent group.

In embodiments, a substituted W² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W² is substituted with a plurality of groups selected from substituent groups, size-limited substituent groups, and lower substituent groups; each substituent group, size-limited substituent group, and/or lower substituent group may optionally be different. In embodiments, when W² is substituted, it is substituted with at least one substituent group. In embodiments, when W² is substituted, it is substituted with at least one size-limited substituent group. In embodiments, when W² is substituted, it is substituted with at least one lower substituent group.

In embodiments, a substituted W³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W³ is substituted with a plurality of groups selected from substituent groups, size-limited substituent groups, and lower substituent groups; each substituent group, size-limited substituent group, and/or lower substituent group may optionally be different. In embodiments, when W³ is substituted, it is substituted with at least one substituent group. In embodiments, when W³ is substituted, it is substituted with at least one size-limited substituent group. In embodiments, when W³ is substituted, it is substituted with at least one lower substituent group.

In embodiments, a substituted W⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W⁴ is substituted with a plurality of groups selected from substituent groups, size-limited substituent groups, and lower substituent groups; each substituent group, size-limited substituent group, and/or lower substituent group may optionally be different. In embodiments, when W⁴ is substituted, it is substituted with at least one substituent group. In embodiments, when W⁴ is substituted, it is substituted with at least one size-limited substituent group. In embodiments, when W⁴ is substituted, it is substituted with at least one lower substituent group.

In embodiments, a substituted W⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W⁵ is substituted with a plurality of groups selected from substituent groups, size-limited substituent groups, and lower substituent groups; each substituent group, size-limited substituent group, and/or lower substituent group may optionally be different. In embodiments, when W⁵ is substituted, it is substituted with at least one substituent group. In embodiments, when W⁵ is substituted, it is substituted with at least one size-limited substituent group. In embodiments, when W⁵ is substituted, it is substituted with at least one lower substituent group.

In embodiments, a substituted W⁶ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted W⁶ is substituted with a plurality of groups selected from substituent groups, size-limited substituent groups, and lower substituent groups; each substituent group, size-limited substituent group, and/or lower substituent group may optionally be different. In embodiments, when W⁶ is substituted, it is substituted with at least one substituent group. In embodiments, when W⁶ is substituted, it is substituted with at least one size-limited substituent group. In embodiments, when W⁶ is substituted, it is substituted with at least one lower substituent group.

In embodiments, W³ is

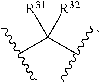

wherein R³¹ and R³² are independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered); or R³¹ and R³² are joined to form a substituted or unsubstituted cycloalkyl (e.g., C₃-C₈, C₃-C₆, C₄-C₆, or C₅-C₆) or substituted or unsubstituted heterocycloalkyl (e.g., 3 to 8 membered, 3 to 6 membered, 4 to 6 membered, 4 to 5 membered, or 5 to 6 membered).

In embodiments, a substituted R³¹ (e.g., substituted alkyl, substituted cycloalkyl, and/or substituted heterocycloalkyl) is substituted with at least one substituent group, size-limited substituent group, or lower substituent group; wherein if the substituted R³¹ is substituted with a plurality of groups selected from substituent groups, size-limited substituent groups, and lower substituent groups; each substituent group, size-limited substituent group, and/or lower substituent group may optionally be different. In embodiments, when R³¹ is substituted, it is substituted with at least one substituent group. In embodiments, when R³¹ is substituted, it is substituted with at least one size-limited substituent group. In embodiments, when R³¹ is substituted, it is substituted with at least one lower substituent group.

In embodiments, a substituted R³² (e.g., substituted alkyl, substituted cycloalkyl, and/or substituted heterocycloalkyl) is substituted with at least one substituent group, size-limited substituent group, or lower substituent group; wherein if the substituted R³² is substituted with a plurality of groups selected from substituent groups, size-limited substituent groups, and lower substituent groups; each substituent group, size-limited substituent group, and/or lower substituent group may optionally be different. In embodiments, when R³² is substituted, it is substituted with at least one substituent group. In embodiments, when R³² is substituted, it is substituted with at least one size-limited substituent group. In embodiments, when R³² is substituted, it is substituted with at least one lower substituent group.

In embodiments, a substituted ring formed when R³¹ and R³² substituents are joined (e.g., substituted cycloalkyl and/or substituted heterocycloalkyl) is substituted with at least one substituent group, size-limited substituent group, or lower substituent group; wherein if the substituted ring formed when R³¹ and R³² substituents are joined is substituted with a plurality of groups selected from substituent groups, size-limited substituent groups, and lower substituent groups; each substituent group, size-limited substituent group, and/or lower substituent group may optionally be different. In embodiments, when the ring formed when R³¹ and R³² substituents are joined is substituted, it is substituted with at least one substituent group. In embodiments, when the ring formed when R³¹ and R³² substituents are joined is substituted, it is substituted with at least one size-limited substituent group. In embodiments, when the ring formed when R³¹ and R³² substituents are joined is substituted, it is substituted with at least one lower substituent group.

In embodiments, R³¹ and R³² are independently hydrogen or unsubstituted C₁-C₄ alkyl. In embodiments, R³¹ and R³² are independently hydrogen. In embodiments, R³¹ and R³² are independently unsubstituted methyl. In embodiments, R³¹ and R³² are independently unsubstituted ethyl. In embodiments, R³¹ and R³² are independently unsubstituted propyl. In embodiments, R³¹ and R³² are independently unsubstituted n-propyl. In embodiments, R³¹ and R³² are independently unsubstituted isopropyl. In embodiments, R³¹ and R³² are independently unsubstituted butyl. In embodiments, R³¹ and R³² are independently unsubstituted n-butyl. In embodiments, R³¹ and R³² are independently unsubstituted isobutyl. In embodiments, R³¹ and R³² are independently unsubstituted tert-butyl. In embodiments, R³¹ and R³² are joined to form a substituted or unsubstituted C₃-C₆ cycloalkyl. In embodiments, R³¹ and R³² are joined to form a substituted or unsubstituted cyclopropyl. In embodiments, R³¹ and R³² are joined to form a substituted or unsubstituted cyclobutyl. In embodiments, R³¹ and R³² are joined to form a substituted or unsubstituted cyclopentyl. In embodiments, R³¹ and R³² are joined to form a substituted or unsubstituted cyclohexyl. In embodiments, R³¹ and R³² are joined to form a substituted or unsubstituted 3 to 6 membered heterocycloalkyl. In embodiments, R³¹ and R³² are joined to form a substituted or unsubstituted tetrahydropyranyl. In embodiments, R³¹ and R³² are joined to form a substituted or unsubstituted piperidinyl.

In embodiments, W¹ is substituted or unsubstituted arylene or substituted or unsubstituted heteroarylene. In embodiments, W¹ is substituted or unsubstituted phenylene. In embodiments, W¹ is chloro-substituted phenylene. In embodiments, W¹ is

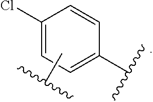

In embodiments, W¹ is methoxy-substituted phenylene. In embodiments, W¹ is

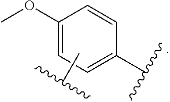

In embodiments, W¹ is methyl-substituted phenylene. In embodiments, W¹ is

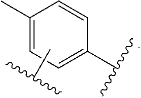

In embodiments, W¹ is

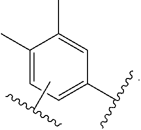

In embodiments, W¹ is

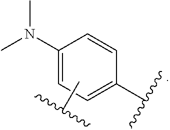

In embodiments, W¹ is unsubstituted phenylene. In embodiments, W¹ is substituted or unsubstituted 5 or 6 membered heteroarylene. In embodiments, W¹ is substituted or unsubstituted pyridinylene. In embodiments, W¹ is chloro-substituted pyridinylene. In embodiments, W¹ is

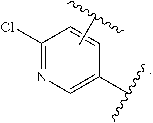

In embodiments, W¹ is

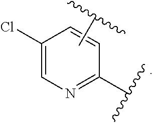

In embodiments, W¹ is unsubstituted pyridinylene. In embodiments, W¹ is substituted or unsubstituted pyrimidinylene. In embodiments, W¹ is chloro-substituted pyrimidinylene. In embodiments, W¹ is

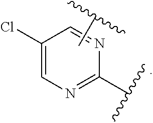

In embodiments, W¹ is

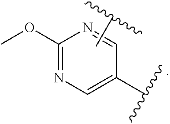

In embodiments, W¹ is unsubstituted pyrimidinylene. In embodiments, W¹ is substituted or unsubstituted pyrazinylene. In embodiments, W¹ is chloro-substituted pyrazinylene. In embodiments, W¹ is

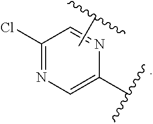

In embodiments, W¹ is unsubstituted pyrazinylene.

In embodiments, W² is —O— or —NH—. In embodiments, W² is —O—. In embodiments, W² is —NH—.

In embodiments, W⁴ is —C(O)—.

In embodiments, W⁵ is substituted or unsubstituted 3 to 8 membered heterocycloalkylene. In embodiments, W⁵ is substituted or unsubstituted 6 membered heterocycloalkylene. In embodiments, W⁵ is substituted or unsubstituted piperidinylene. In embodiments, W⁵ is unsubstituted piperidinylene. In embodiments, W⁵ is

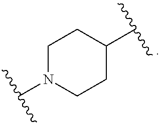 [SMILES: *C1CCN(*)CC1]

In embodiments, W⁶ is a bond.

In embodiments, —W²—W³—W⁴—W⁵— is

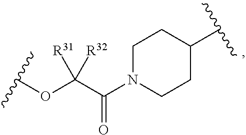

wherein R³¹ and R³² are as described herein, including in embodiments. In embodiments, —W²—W³—W⁴—W⁵— is

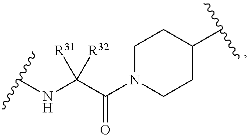

wherein R³¹ and R³² are as described herein, including in embodiments.

In embodiments, W is a bond, substituted or unsubstituted C₁-C₂₀ alkylene, substituted or unsubstituted 2 to 20 membered heteroalkylene, substituted or unsubstituted C₃-C₁₂ cycloalkylene, substituted or unsubstituted 3 to 12 membered heterocycloalkylene, substituted or unsubstituted C₆-C₁₂ arylene, or substituted or unsubstituted 5 to 12 membered heteroarylene. In embodiments, W is a bond, substituted or unsubstituted C₁-C₁₂ alkylene, substituted or unsubstituted 2 to 12 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, W is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 9 membered heteroarylene. In embodiments, W is a bond, substituted or unsubstituted C₁-C₆ alkylene, substituted or unsubstituted 2 to 6 membered heteroalkylene, substituted or unsubstituted C3-C₆ cycloalkylene, substituted or unsubstituted 3 to 6 membered heterocycloalkylene, substituted or unsubstituted phenylene, or substituted or unsubstituted 5 to 6 membered heteroarylene. In embodiments, W is a bond. In embodiments, W is a substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

In embodiments, W is a bond. In embodiments, W is substituted alkylene. In embodiments, W is substituted heteroalkylene. In embodiments, W is substituted cycloalkylene. In embodiments, W is substituted heterocycloalkylene. In embodiments, W is substituted arylene. In embodiments, W is substituted heteroarylene. In embodiments, W is substituted C₁-C₂₀ alkylene. In embodiments, W is substituted 2 to 20 membered heteroalkylene. In embodiments, W is substituted C₃-C₁₂ cycloalkylene. In embodiments, W is substituted 3 to 12 membered heterocycloalkylene. In embodiments, W is substituted C₆-C₁₂ arylene. In embodiments, W is substituted 5 to 12 membered heteroarylene. In embodiments, W is substituted C₁-C₁₂ alkylene. In embodiments, W is substituted 2 to 12 membered heteroalkylene. In embodiments, W is substituted C₃-C₈ cycloalkylene. In embodiments, W is substituted 3 to 8 membered heterocycloalkylene. In embodiments, W is substituted C₆-C₁₀ arylene. In embodiments, W is substituted 5 to 10 membered heteroarylene. In embodiments, W is substituted C₁-C₈ alkylene. In embodiments, W is substituted 2 to 8 membered heteroalkylene. In embodiments, W is substituted C₃-C₈ cycloalkylene. In embodiments, W is substituted 3 to 8 membered heterocycloalkylene. In embodiments, W is substituted phenylene. In embodiments, W is substituted 5 to 9 membered heteroarylene. In embodiments, W is substituted C₁-C₆ alkylene. In embodiments, W is substituted 2 to 6 membered heteroalkylene. In embodiments, W is substituted C₃-C₆ cycloalkylene. In embodiments, W is substituted 3 to 6 membered heterocycloalkylene. In embodiments, W is substituted phenylene. In embodiments, W is substituted 5 to 6 membered heteroarylene. In embodiments, W is substituted C₁-C₆ alkylene. In embodiments, W is substituted 2 to 6 membered heteroalkylene. In embodiments, W is substituted C₃-C₆ cycloalkylene. In embodiments, W is substituted 3 to 6 membered heterocycloalkylene. In embodiments, W is substituted phenylene. In embodiments, W is substituted 5 to 6 membered heteroarylene.

In embodiments, W is a bond. In embodiments, W is unsubstituted alkylene. In embodiments, W is unsubstituted heteroalkylene. In embodiments, W is unsubstituted cycloalkylene. In embodiments, W is unsubstituted heterocycloalkylene. In embodiments, W is unsubstituted arylene. In embodiments, W is unsubstituted heteroarylene. In embodiments, W is unsubstituted C₁-C₂₀ alkylene. In embodiments, W is unsubstituted 2 to 20 membered heteroalkylene. In embodiments, W is unsubstituted C₃-C₁₂ cycloalkylene. In embodiments, W is unsubstituted 3 to 12 membered heterocycloalkylene. In embodiments, W is unsubstituted C₆-C₁₂ arylene. In embodiments, W is unsubstituted 5 to 12 membered heteroarylene. In embodiments, W is unsubstituted C₁-C₁₂ alkylene. In embodiments, W is unsubstituted 2 to 12 membered heteroalkylene. In embodiments, W is unsubstituted C₃-C₈ cycloalkylene. In embodiments, W is unsubstituted 3 to 8 membered heterocycloalkylene. In embodiments, W is unsubstituted C₆-C₁₀ arylene. In embodiments, W is unsubstituted 5 to 10 membered heteroarylene. In embodiments, W is unsubstituted C₁-C₈ alkylene. In embodiments, W is unsubstituted 2 to 8 membered heteroalkylene. In embodiments, W is unsubstituted C₃-C₈ cycloalkylene. In embodiments, W is unsubstituted 3 to 8 membered heterocycloalkylene. In embodiments, W is unsubstituted phenylene. In embodiments, W is unsubstituted 5 to 9 membered heteroarylene. In embodiments, W is unsubstituted C₁-C₆ alkylene. In embodiments, W is unsubstituted 2 to 6 membered heteroalkylene. In embodiments, W is unsubstituted C₃-C₆ cycloalkylene. In embodiments, W is unsubstituted 3 to 6 membered heterocycloalkylene. In embodiments, W is unsubstituted phenylene. In embodiments, W is unsubstituted 5 to 6 membered heteroarylene. In embodiments, W is unsubstituted C₁-C₆ alkylene. In embodiments, W is unsubstituted 2 to 6 membered heteroalkylene. In embodiments, W is unsubstituted C₃-C₆ cycloalkylene. In embodiments, W is unsubstituted 3 to 6 membered heterocycloalkylene. In embodiments, W is unsubstituted phenylene. In embodiments, W is unsubstituted 5 to 6 membered heteroarylene.

In embodiments, W is a bond. In embodiments, W is (-L⁵-R⁵)-substituted alkylene. In embodiments, W is (-L⁵-R⁵)-substituted heteroalkylene. In embodiments, W is (-L⁵-R⁵)-substituted cycloalkylene. In embodiments, W is (-L-R⁵)-substituted heterocycloalkylene. In embodiments, W is (-L⁵-R⁵)-substituted arylene. In embodiments, W is (-L⁵-R⁵)-substituted heteroarylene. In embodiments, W is (-L⁵-R⁵)-substituted C₁-C₂₀ alkylene. In embodiments, W is (-L⁵-R⁵)-substituted 2 to 20 membered heteroalkylene. In embodiments, W is (-L⁵-R⁵)-substituted C₃-C₁₂ cycloalkylene. In embodiments, W is (-L⁵-R⁵)-substituted 3 to 12 membered heterocycloalkylene. In embodiments, W is (-L⁵-R⁵)-substituted C₆-C₁₂ arylene. In embodiments, W is (-L⁵-R⁵)-substituted 5 to 12 membered heteroarylene. In embodiments, W is (-L⁵-R⁵)-substituted C₁-C₁₂ alkylene. In embodiments, W is (-L⁵-R⁵)-substituted 2 to 12 membered heteroalkylene. In embodiments, W is (-L⁵-R⁵)-substituted C₃-C₈ cycloalkylene. In embodiments, W is (-L⁵-R⁵)-substituted 3 to 8 membered heterocycloalkylene. In embodiments, W is (-L⁵-R⁵)-substituted C₆-C₁₀ arylene. In embodiments, W is (-L⁵-R⁵)-substituted 5 to 10 membered heteroarylene. In embodiments, W is (-L⁵-R⁵)-substituted C₁-C₈ alkylene. In embodiments, W is (-L⁵-R⁵)-substituted 2 to 8 membered heteroalkylene. In embodiments, W is (-L⁵-R⁵)-substituted C₃-C₈ cycloalkylene. In embodiments, W is (-L⁵-R⁵)-substituted 3 to 8 membered heterocycloalkylene. In embodiments, W is (-L⁵-R⁵)-substituted phenylene. In embodiments, W is (-L⁵-R⁵)-substituted 5 to 9 membered heteroarylene. In embodiments, W is (-L⁵-R⁵)-substituted C₁-C₆ alkylene. In embodiments, W is (-L⁵-R⁵)-substituted 2 to 6 membered heteroalkylene. In embodiments, W is (-L⁵-R⁵)-substituted C₃-C₆ cycloalkylene. In embodiments, W is (-L⁵-R⁵)-substituted 3 to 6 membered heterocycloalkylene. In embodiments, W is (-L⁵-R⁵)-substituted phenylene. In embodiments, W is (-L⁵-R⁵)-substituted 5 to 6 membered heteroarylene. In embodiments, W is (-L⁵-R⁵)-substituted C₁-C₆ alkylene. In embodiments, W is (-L⁵-R⁵)-substituted 2 to 6 membered heteroalkylene. In embodiments, W is (-L⁵-R⁵)-substituted C₃-C₆ cycloalkylene. In embodiments, W is (-L⁵-R⁵)-substituted 3 to 6 membered heterocycloalkylene. In embodiments, W is (-L⁵-R⁵)-substituted phenylene. In embodiments, W is (-L⁵-R⁵)-substituted 5 to 6 membered heteroarylene.

In embodiments, W is a bond. In embodiments, W is (-L¹-R¹)-substituted alkylene. In embodiments, W is (-L¹-R¹)-substituted heteroalkylene. In embodiments, W is (-L¹-R¹)-substituted cycloalkylene. In embodiments, W is (-L¹-R¹)-substituted heterocycloalkylene. In embodiments, W is (-L¹-R¹)-substituted arylene. In embodiments, W is (-L¹-R¹)-substituted heteroarylene. In embodiments, W is (-L¹-R¹)-substituted C₁-C₂₀ alkylene. In embodiments, W is (-L¹-R¹)-substituted 2 to 20 membered heteroalkylene. In embodiments, W is (-L¹-R¹)-substituted C₃-C₁₂ cycloalkylene. In embodiments, W is (-L¹-R¹)-substituted 3 to 12 membered heterocycloalkylene. In embodiments, W is (-L¹-R¹)-substituted C₆-C₁₂ arylene. In embodiments, W is (-L¹-R¹)-substituted 5 to 12 membered heteroarylene. In embodiments, W is (-L¹-R¹)-substituted C₁-C₁₂ alkylene. In embodiments, W is (-L¹-R¹)-substituted 2 to 12 membered heteroalkylene. In embodiments, W is (-L¹-R¹)-substituted C₃-C₈ cycloalkylene. In embodiments, W is (-L¹-R¹)-substituted 3 to 8 membered heterocycloalkylene. In embodiments, W is (-L¹-R¹)-substituted C₆-C₁₀ arylene. In embodiments, W is (-L¹-R¹)-substituted 5 to 10 membered heteroarylene. In embodiments, W is (-L¹-R¹)-substituted C₁-C₈ alkylene. In embodiments, W is (-L¹-R¹)-substituted 2 to 8 membered heteroalkylene. In embodiments, W is (-L¹-R¹)-substituted C₃-C₈ cycloalkylene. In embodiments, W is (-L¹-R¹)-substituted 3 to 8 membered heterocycloalkylene. In embodiments, W is (-L¹-R¹)-substituted phenylene. In embodiments, W is (-L¹-R¹)-substituted 5 to 9 membered heteroarylene. In embodiments, W is (-L¹-R¹)-substituted C₁-C₆ alkylene. In embodiments, W is (-L¹-R¹)-substituted 2 to 6 membered heteroalkylene. In embodiments, W is (-L¹-R¹)-substituted C₃-C₆ cycloalkylene. In embodiments, W is (-L¹-R¹)-substituted 3 to 6 membered heterocycloalkylene. In embodiments, W is (-L¹-R¹)-substituted phenylene. In embodiments, W is (-L¹-R¹)-substituted 5 to 6 membered heteroarylene. In embodiments, W is (-L¹-R¹)-substituted C₁-C₆ alkylene. In embodiments, W is (-L¹-R¹)-substituted 2 to 6 membered heteroalkylene. In embodiments, W is (-L¹-R¹)-substituted C₃-C₆ cycloalkylene. In embodiments, W is (-L¹-R¹)-substituted 3 to 6 membered heterocycloalkylene. In embodiments, W is (-L¹-R¹)-substituted phenylene. In embodiments, W is (-L¹-R¹)-substituted 5 to 6 membered heteroarylene.

In embodiments, W is a bond. In embodiments, W is (-L²-R²)-substituted alkylene. In embodiments, W is (-L²-R²)-substituted heteroalkylene. In embodiments, W is (-L²-R²)-substituted cycloalkylene. In embodiments, W is (-L²-R²)-substituted heterocycloalkylene. In embodiments, W is (-L²-R²)-substituted arylene. In embodiments, W is (-L²-R²)-substituted heteroarylene. In embodiments, W is (-L²-R²)-substituted C₁-C₂₀ alkylene. In embodiments, W is (-L²-R²)-substituted 2 to 20 membered heteroalkylene. In embodiments, W is (-L²-R²)-substituted C₃-C₁₂ cycloalkylene. In embodiments, W is (-L²-R²)-substituted 3 to 12 membered heterocycloalkylene. In embodiments, W is (-L²-R²)-substituted C₆-C₁₂ arylene. In embodiments, W is (-L²-R²)-substituted 5 to 12 membered heteroarylene. In embodiments, W is (-L²-R²)-substituted C₁-C₁₂ alkylene. In embodiments, W is (-L²-R²)-substituted 2 to 12 membered heteroalkylene. In embodiments, W is (-L²-R²)-substituted C₃-C₈ cycloalkylene. In embodiments, W is (-L²-R²)-substituted 3 to 8 membered heterocycloalkylene. In embodiments, W is (-L²-R²)-substituted C₆-C₁₀ arylene. In embodiments, W is (-L²-R²)-substituted 5 to 10 membered heteroarylene. In embodiments, W is (-L²-R²)-substituted C₁-C₈ alkylene. In embodiments, W is (-L²-R²)-substituted 2 to 8 membered heteroalkylene. In embodiments, W is (-L²-R²)-substituted C₃-C₈ cycloalkylene. In embodiments, W is (-L²-R²)-substituted 3 to 8 membered heterocycloalkylene. In embodiments, W is (-L²-R²)-substituted phenylene. In embodiments, W is (-L²-R²)-substituted 5 to 9 membered heteroarylene. In embodiments, W is (-L²-R²)-substituted C₁-C₆ alkylene. In embodiments, W is (-L²-R²)-substituted 2 to 6 membered heteroalkylene. In embodiments, W is (-L²-R²)-substituted C₃-C₆ cycloalkylene. In embodiments, W is (-L²-R²)-substituted 3 to 6 membered heterocycloalkylene. In embodiments, W is (-L²-R²)-substituted phenylene. In embodiments, W is (-L²-R²)-substituted 5 to 6 membered heteroarylene. In embodiments, W is (-L²-R²)-substituted C₁-C₆ alkylene. In embodiments, W is (-L²-R²)-substituted 2 to 6 membered heteroalkylene. In embodiments, W is (-L²-R²)-substituted C₃-C₆ cycloalkylene. In embodiments, W is (-L²-R²)-substituted 3 to 6 membered heterocycloalkylene. In embodiments, W is (-L²-R²)-substituted phenylene. In embodiments, W is (-L²-R²)-substituted 5 to 6 membered heteroarylene.

In embodiments, W is substituted, (-L⁵-R⁵)-substituted, (-L¹-R¹)-substituted, (-L²-R²)-substituted, or unsubstituted

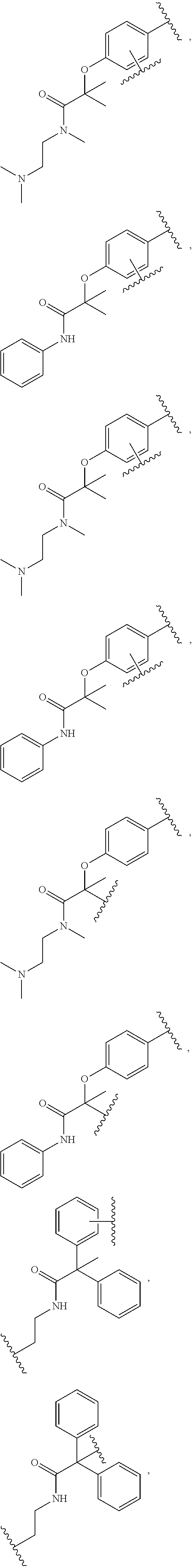

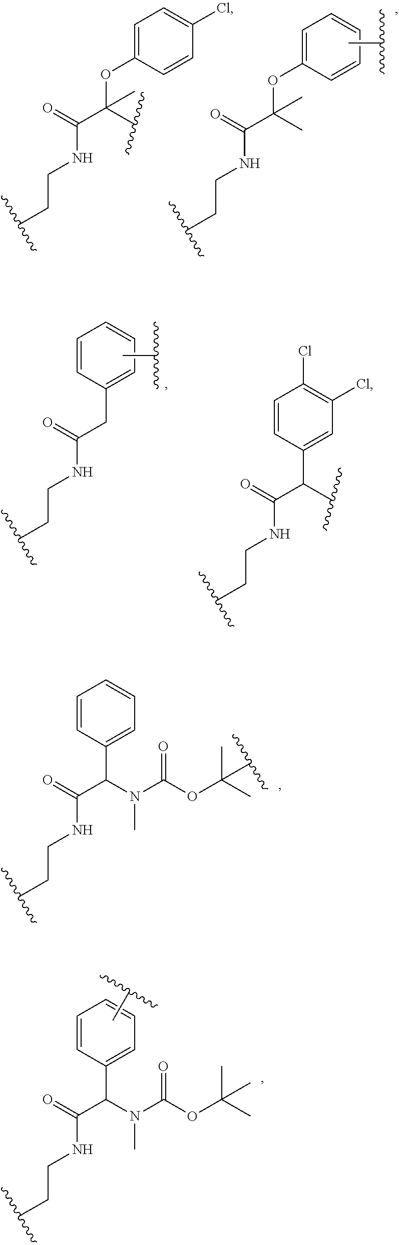

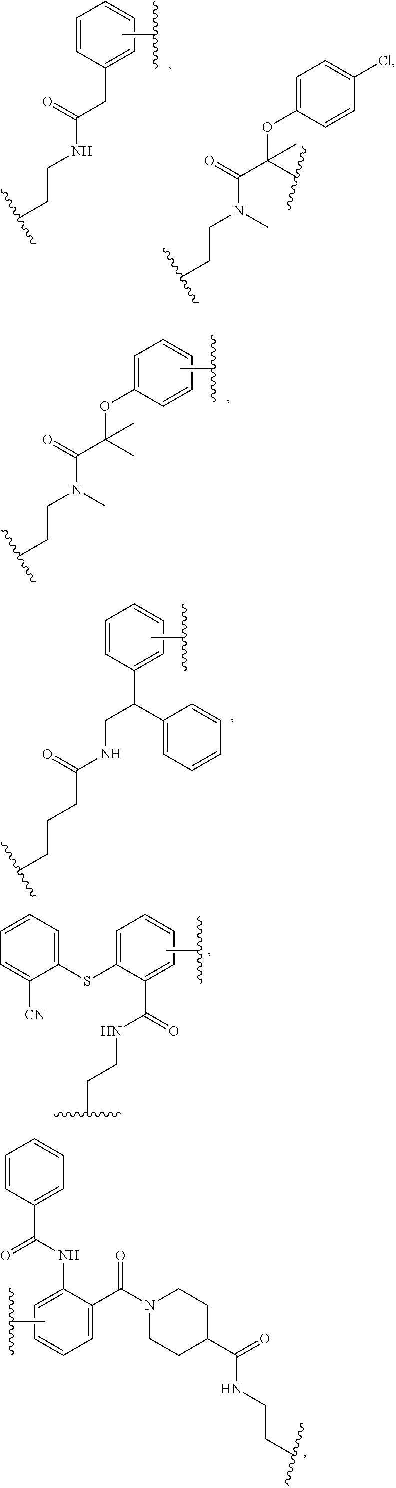

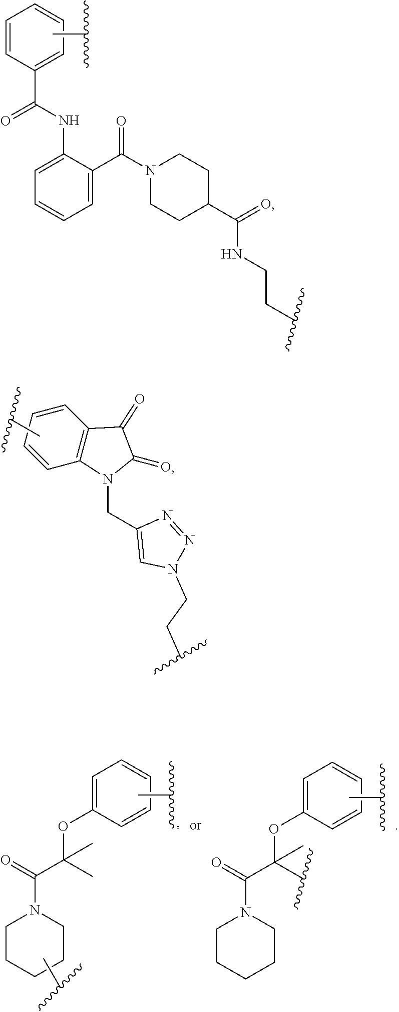

In embodiments, W is substituted, (-L⁵-R⁵)-substituted, or unsubstituted

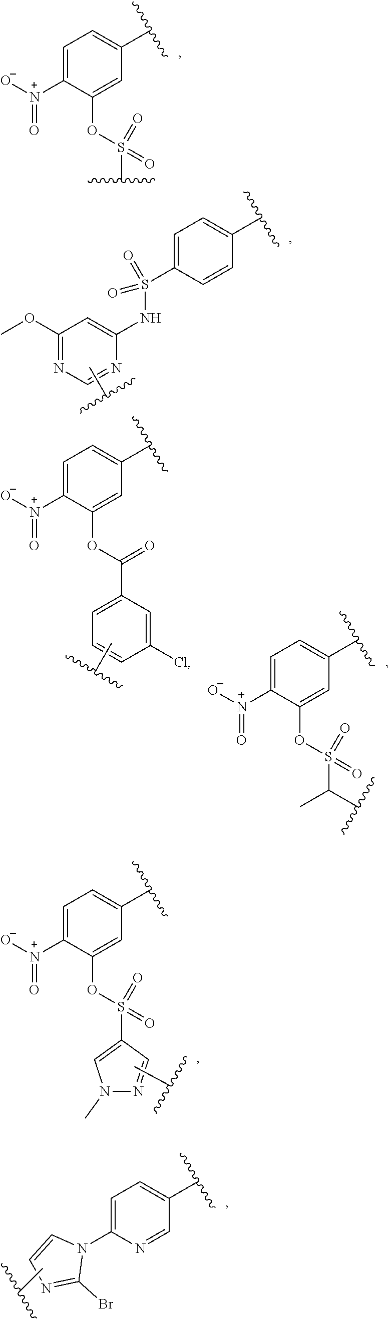

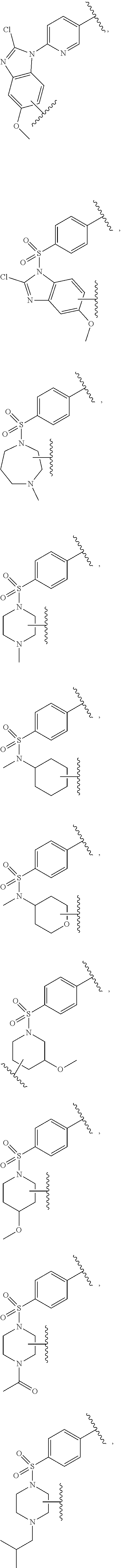

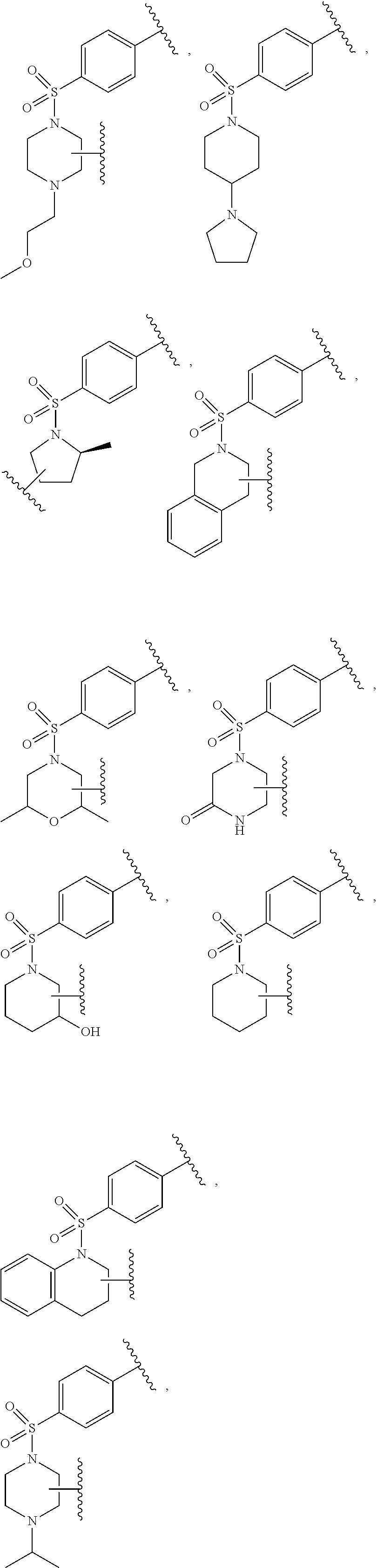

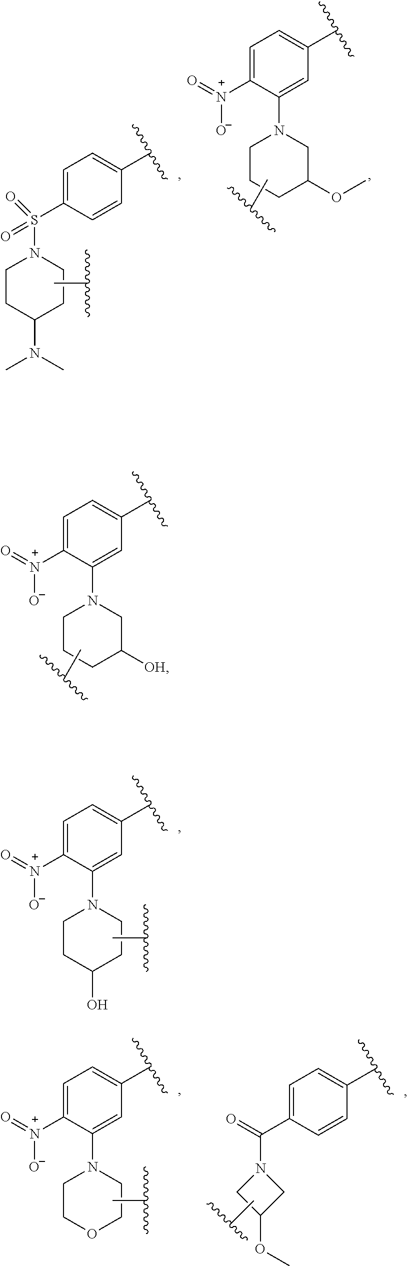

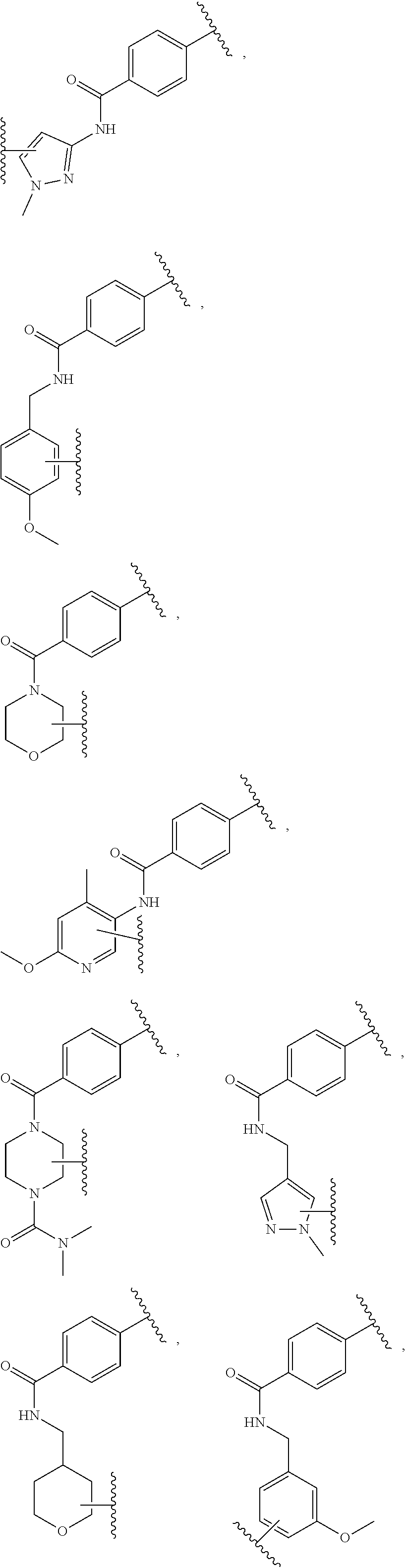

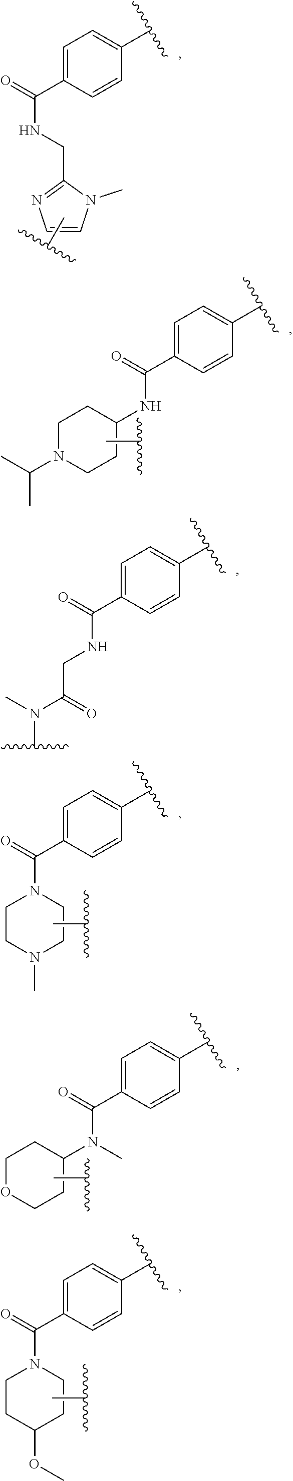

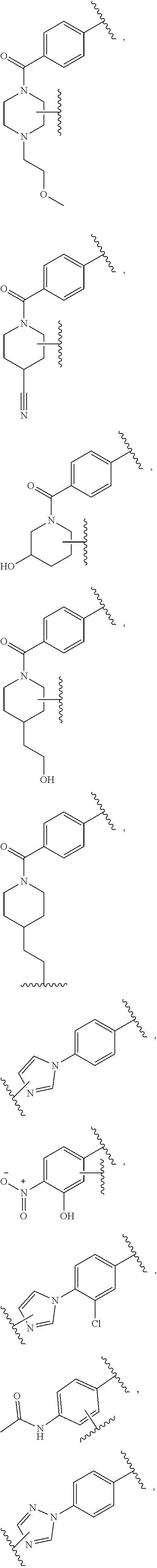

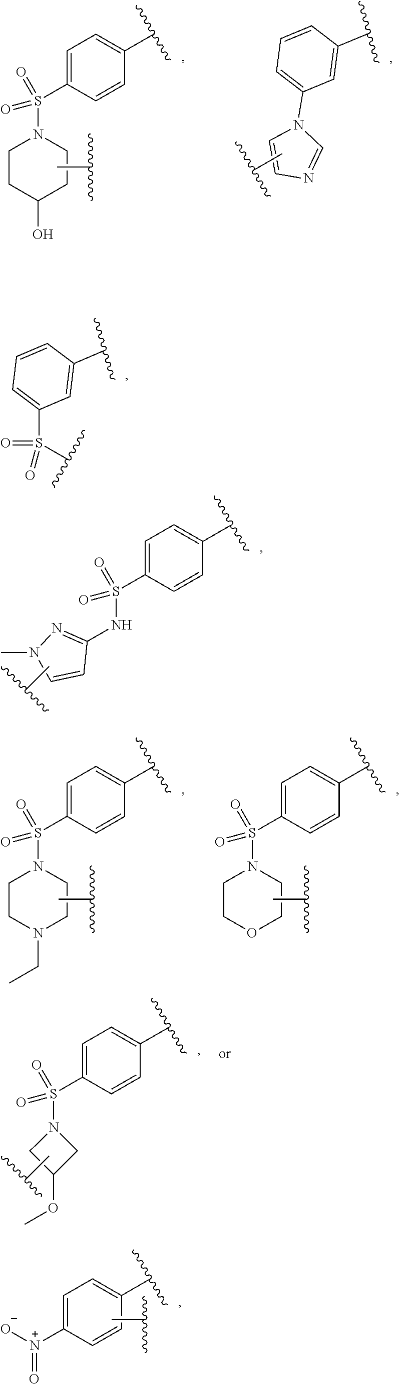

In embodiments, W is substituted, (-L⁵-R⁵)-substituted, or unsubstituted

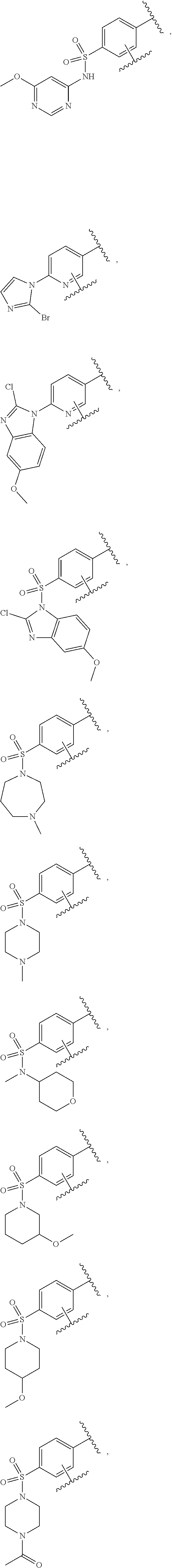

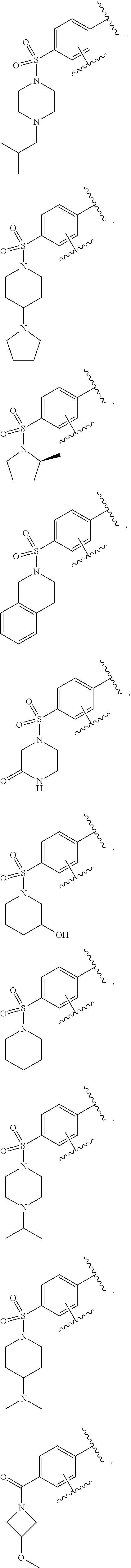

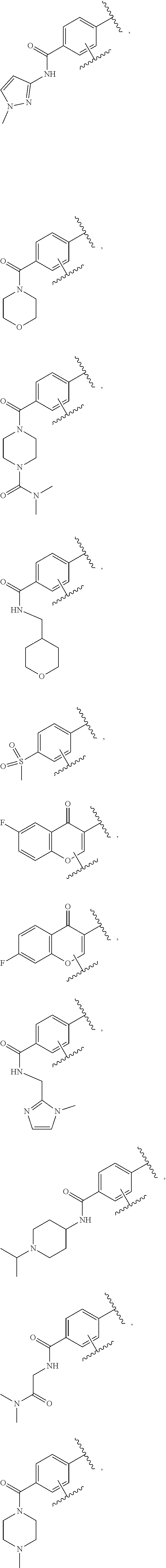

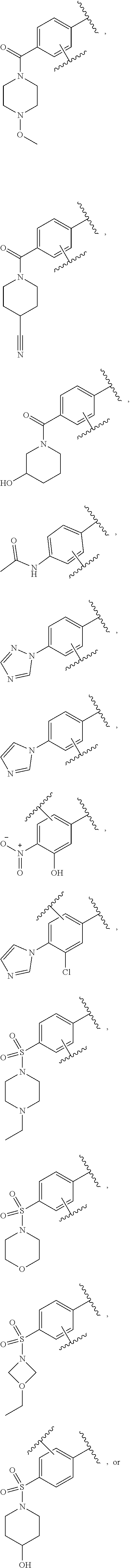

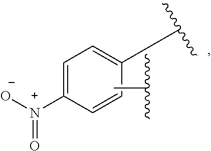

In embodiments, W is substituted, (-L⁵-R⁵)-substituted, (-L¹-R¹)-substituted, (-L²-R²)-substituted, or unsubstituted

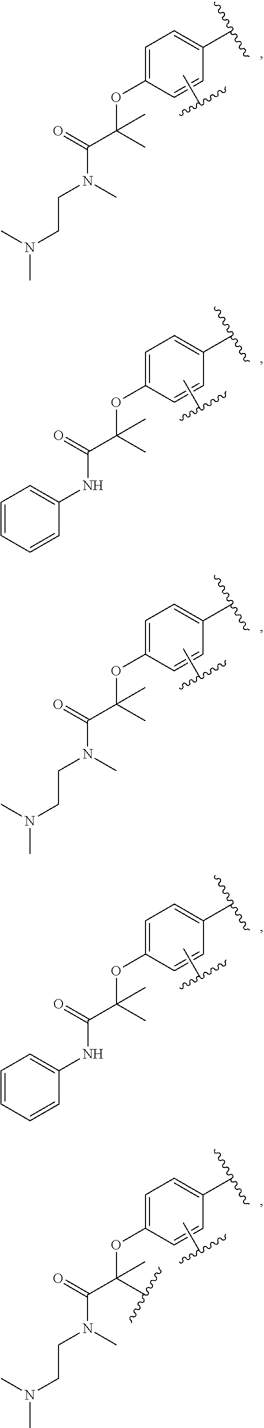

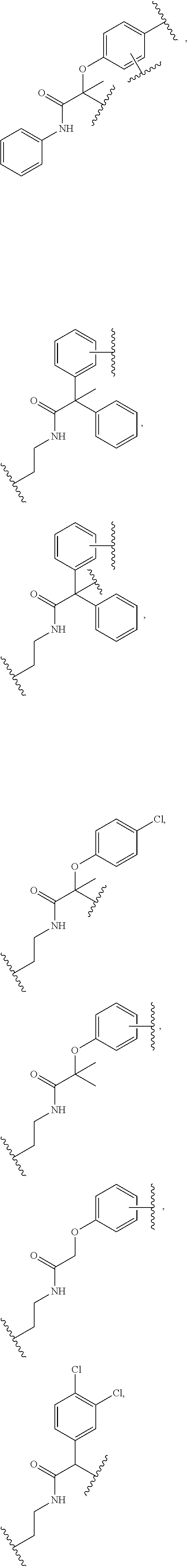

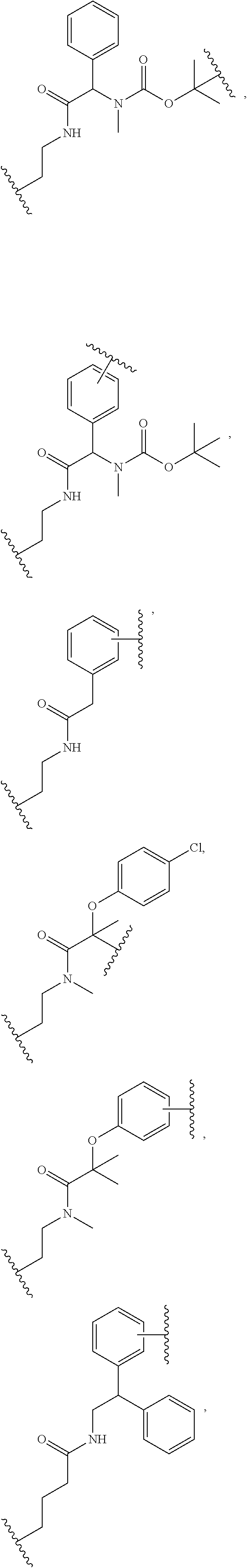

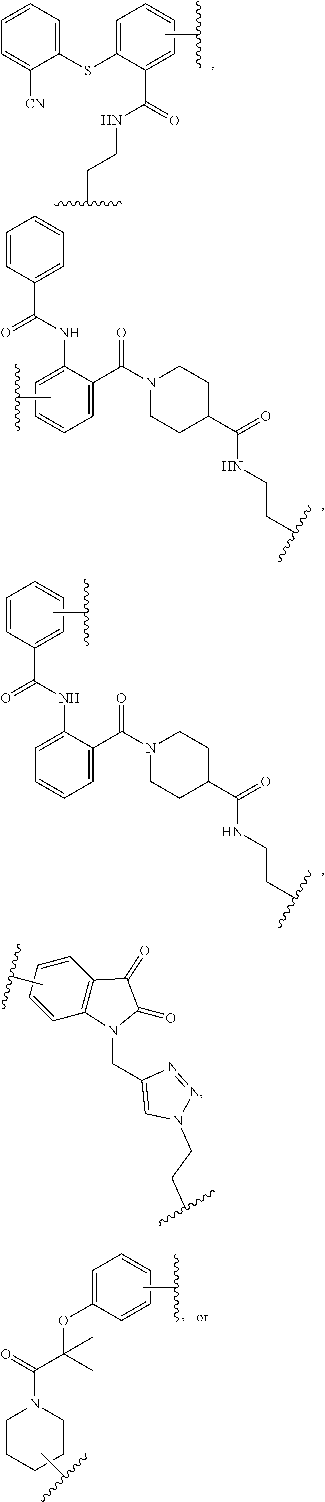

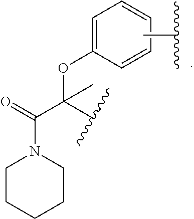

In embodiments, W is substituted, (-L⁵-R⁵)-substituted, (-L¹-R¹)-substituted, (-L²-R²)-substituted, or unsubstituted

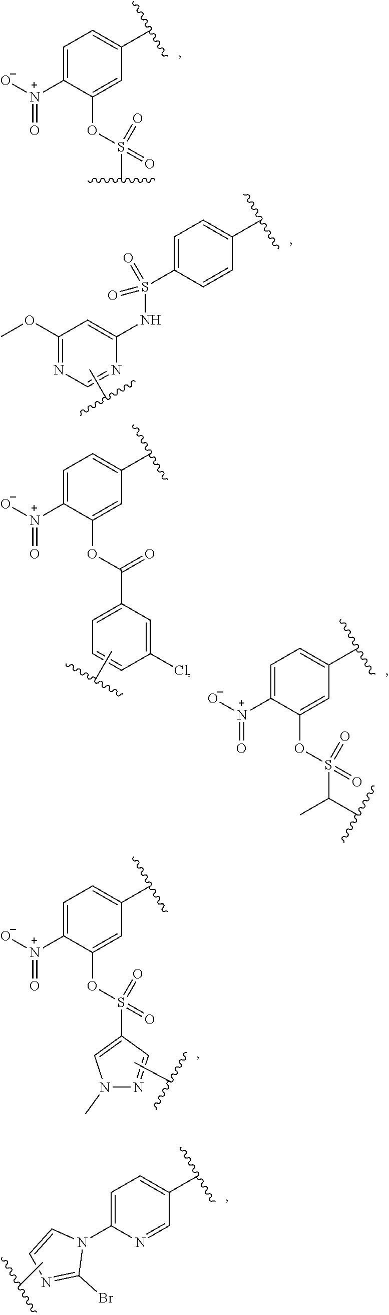

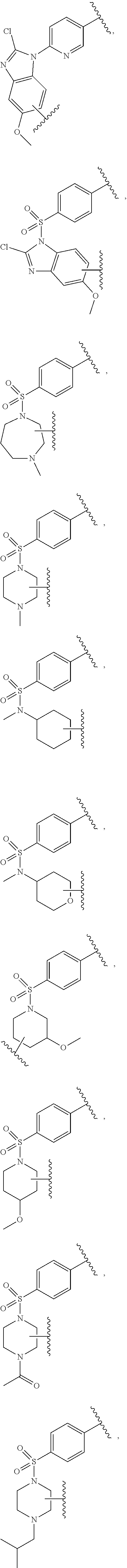

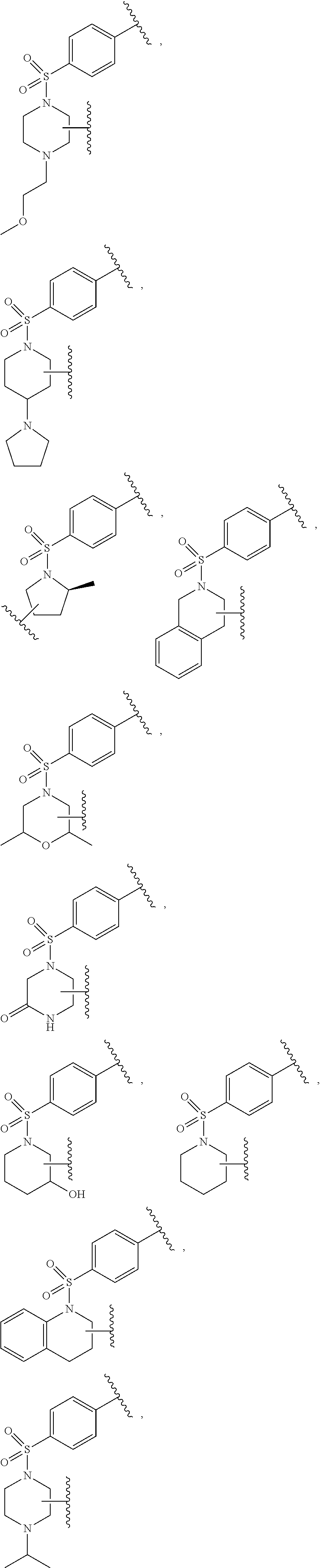

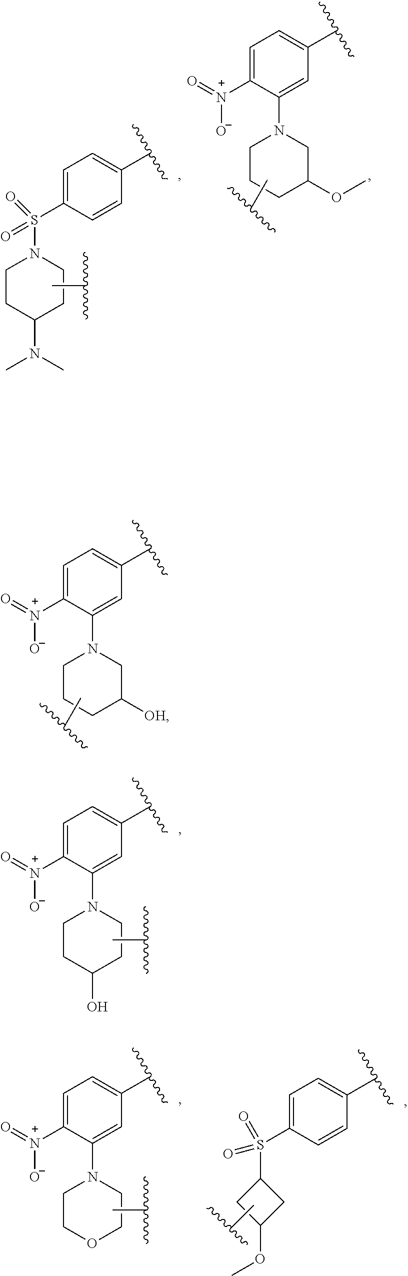

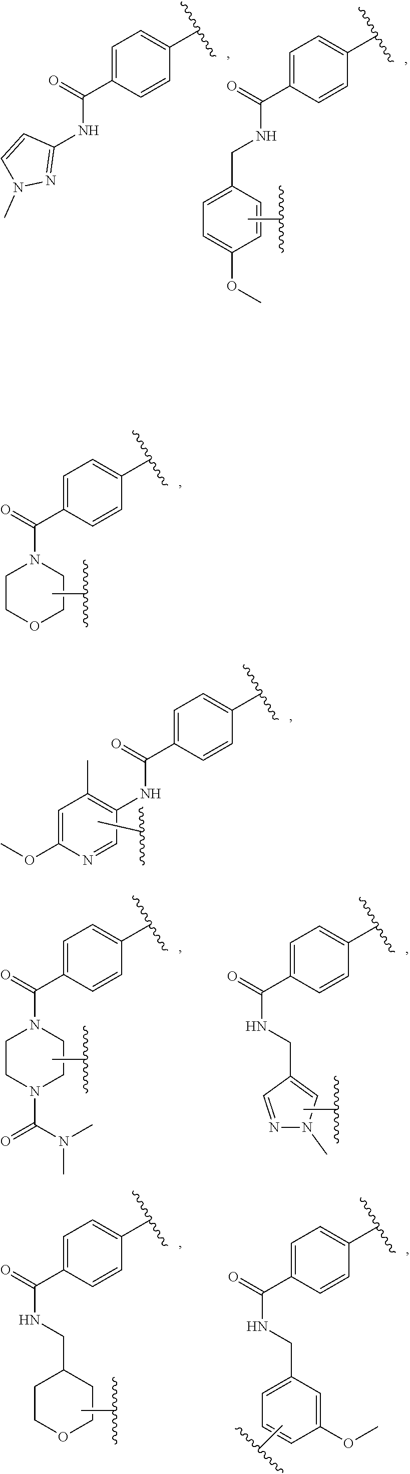

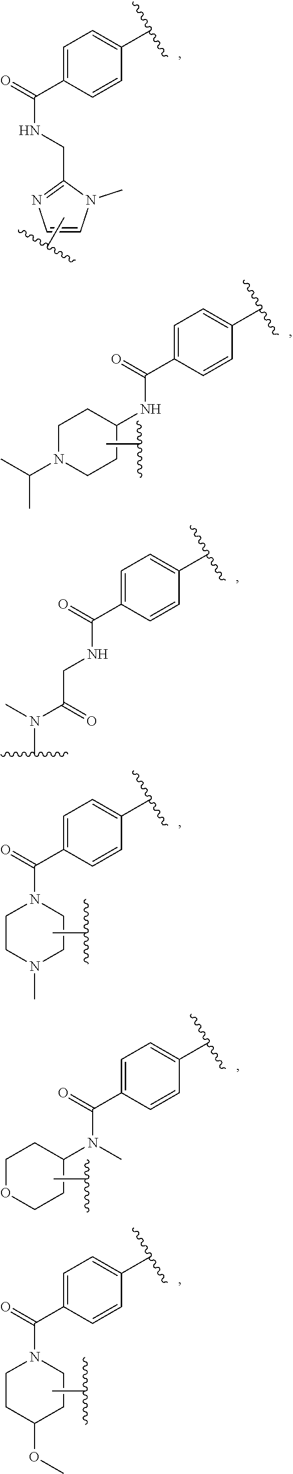

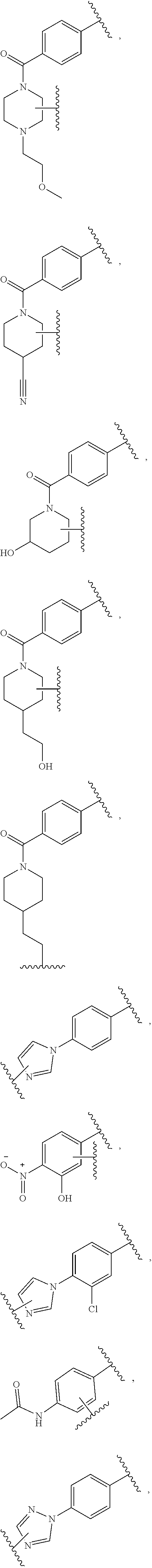

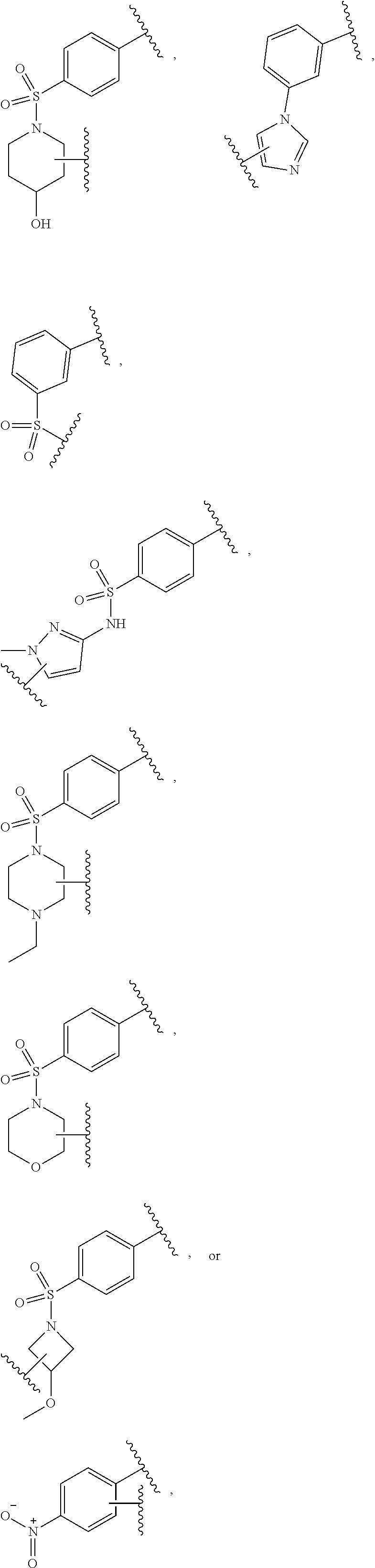

In embodiments, W is substituted, (-L⁵-R⁵)-substituted, (-L¹-R¹)-substituted, (-L²-R²)-substituted, or unsubstituted

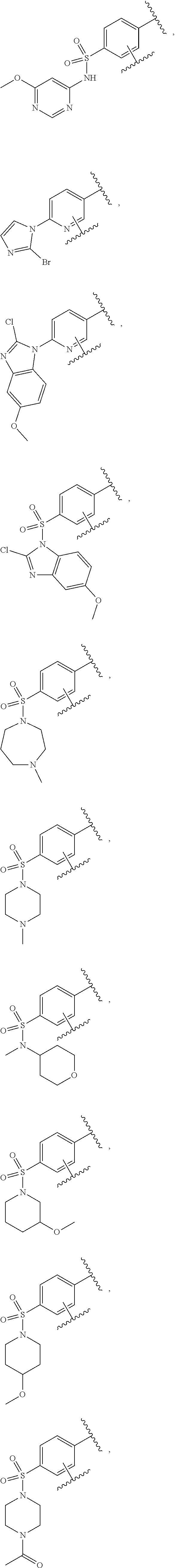

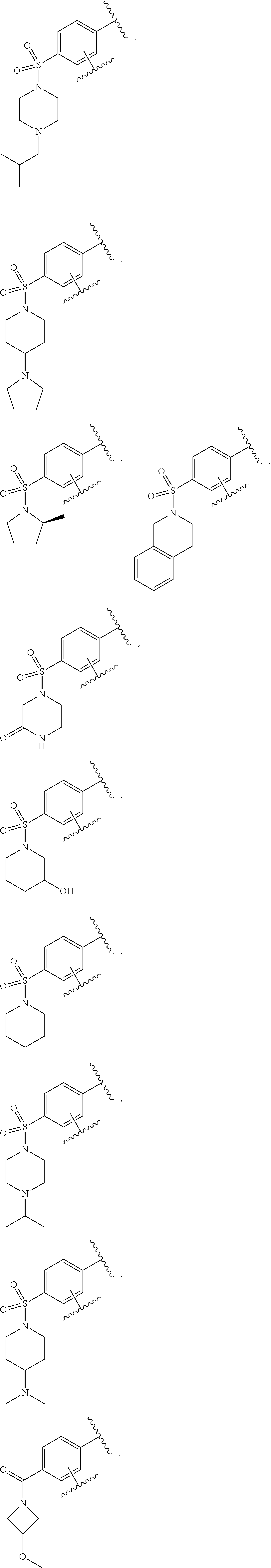

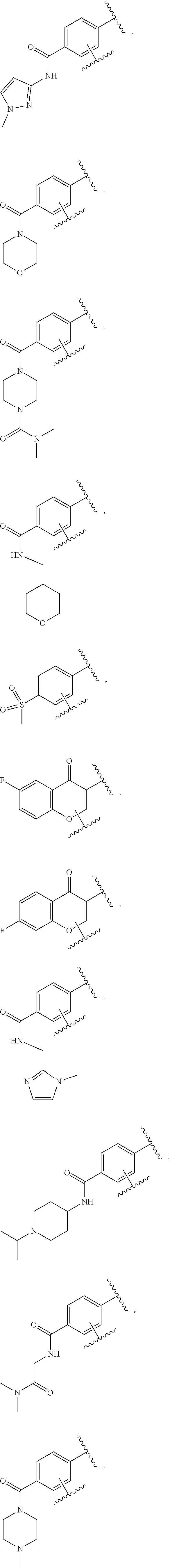

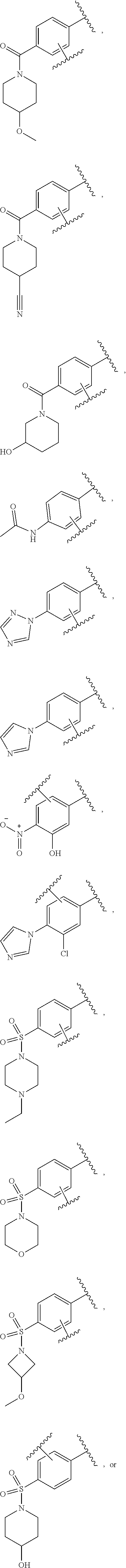

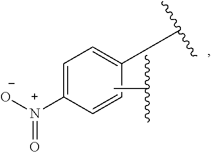

In embodiments, W is substituted, (-L⁵-R⁵)-substituted, (-L¹-R¹)-substituted, (-L²-R²)-substituted, or unsubstituted

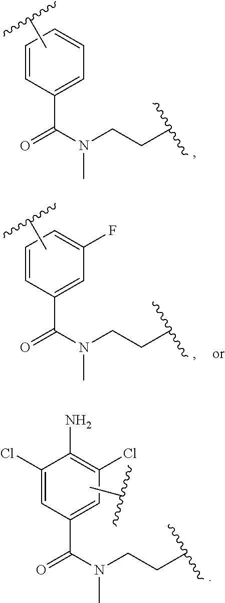

In embodiments, W is substituted, (-L⁵-R⁵)-substituted, (-L¹-R¹)-substituted, (-L²R²)-substituted, or unsubstituted

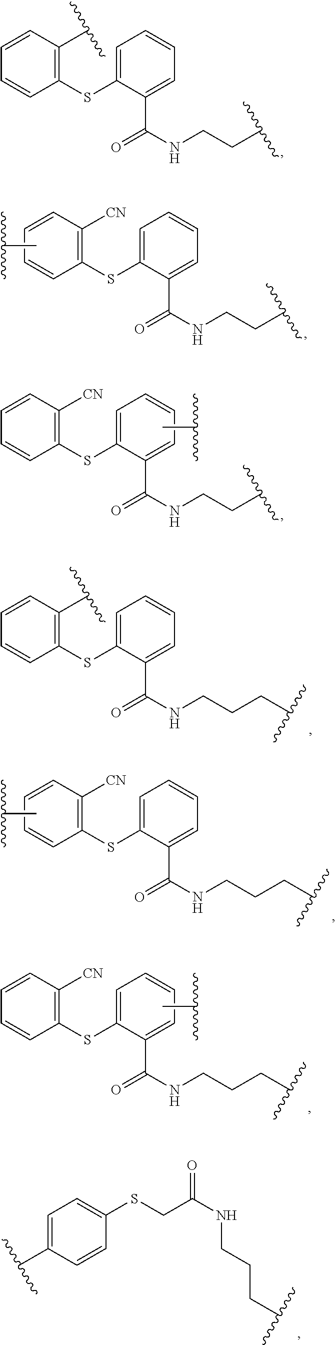

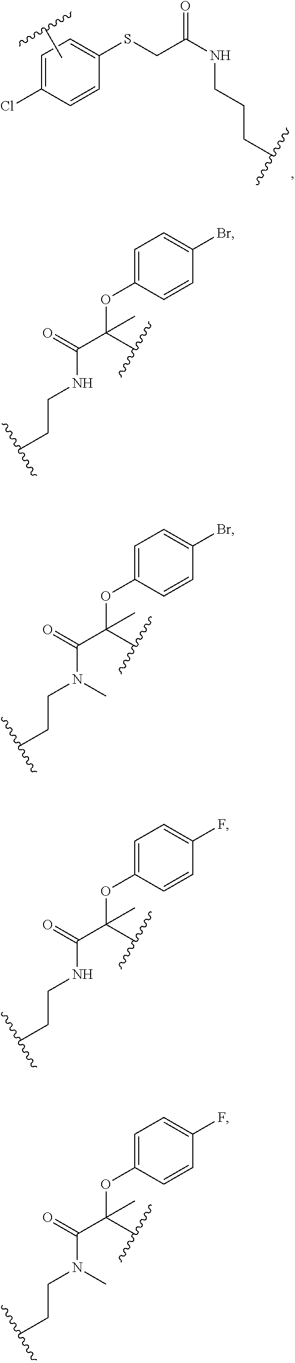

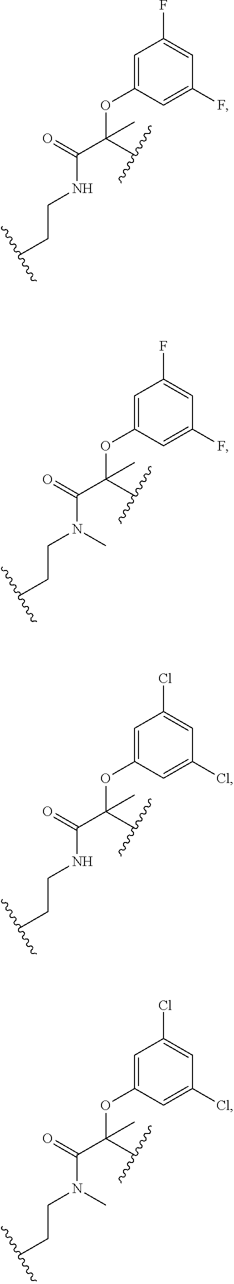 [SMILES: *CCNC(=O)C(*)(C)Oc1cc(F)cc(F)c1 ; *CCN(C)C(=O)C(*)(C)Oc1cc(F)cc(F)c1 ; *CCNC(=O)C(*)(C)Oc1cc(Cl)cc(Cl)c1 ; *CCN(C)C(=O)C(*)(C)Oc1cc(Cl)cc(Cl)c1]

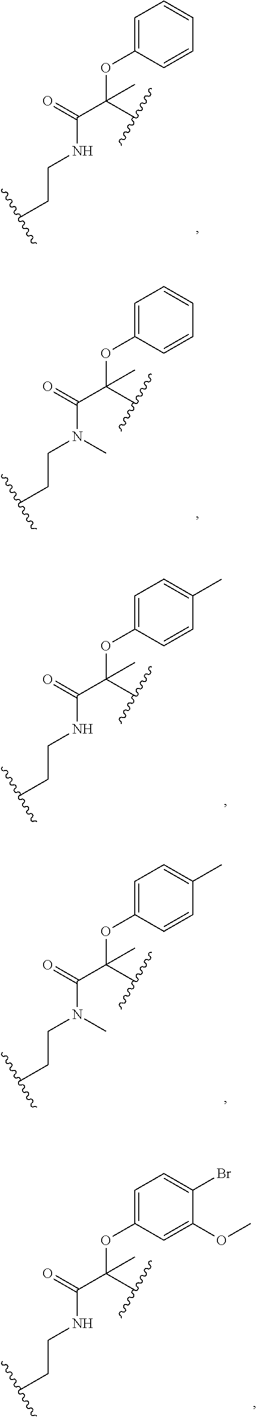 [SMILES: *CCNC(=O)C(*)(C)Oc1ccccc1 ; *CCN(C)C(=O)C(*)(C)Oc1ccccc1 ; *CCNC(=O)C(*)(C)Oc1ccc(C)cc1 ; *CCN(C)C(=O)C(*)(C)Oc1ccc(C)cc1 ; *CCNC(=O)C(*)(C)Oc1ccc(Br)c(OC)c1]

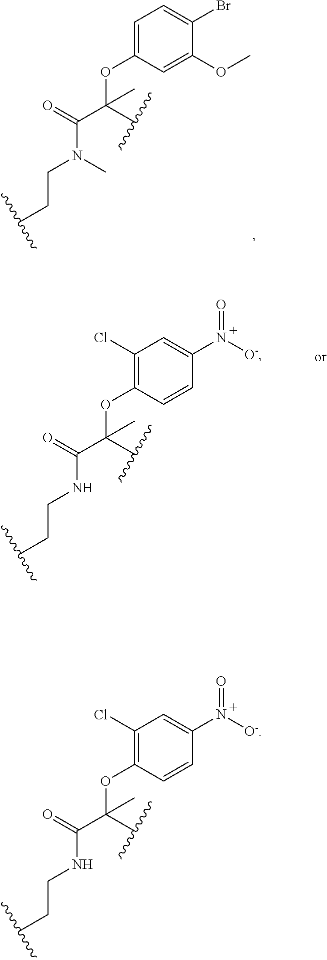

In embodiments, W is substituted, (-L⁵-R⁵)-substituted, (-L¹-R¹)-substituted, (-L²-R²)-substituted, or unsubstituted

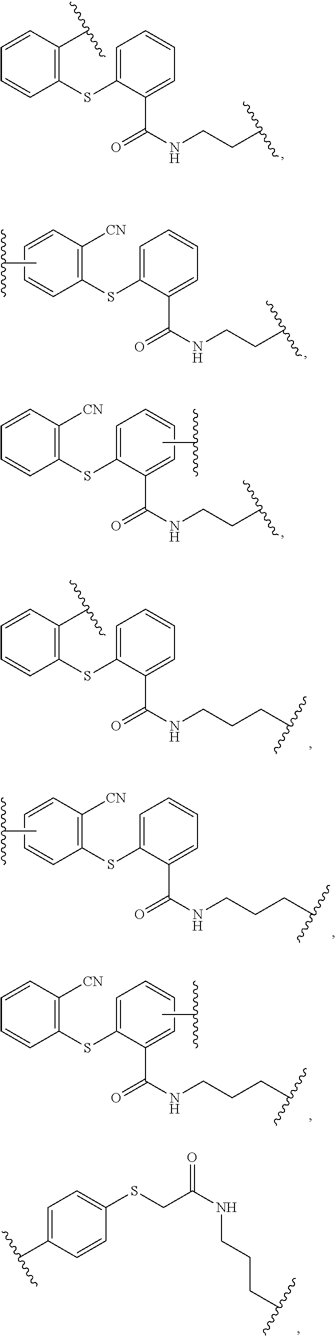

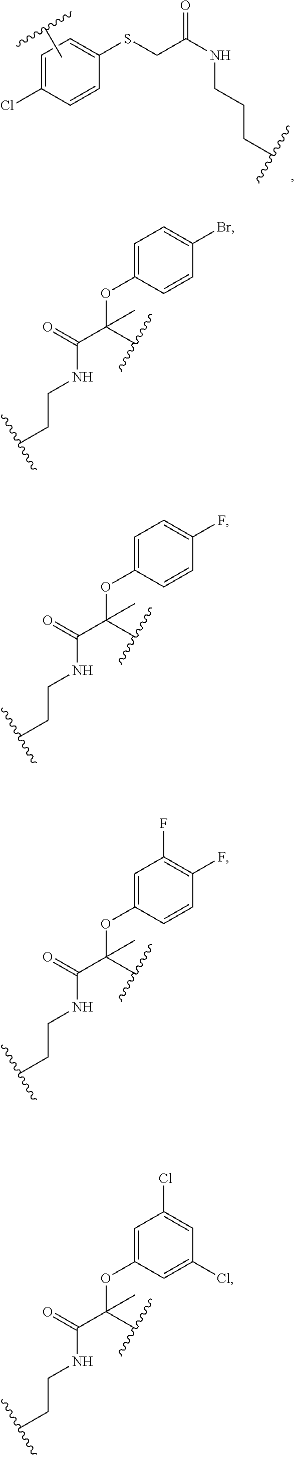

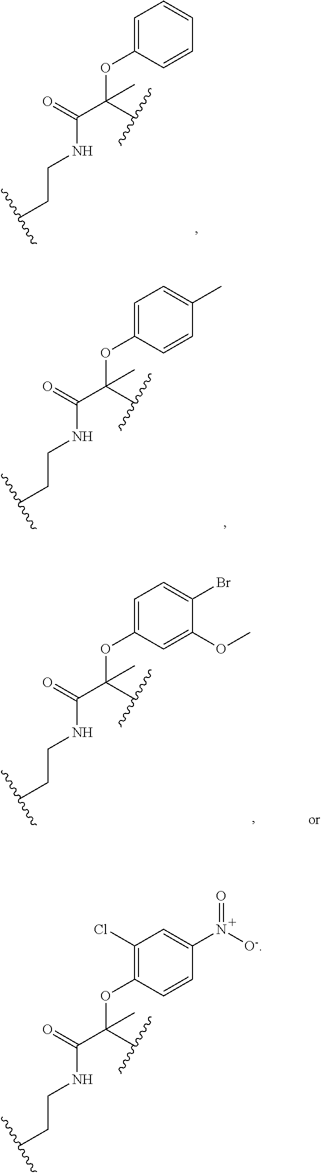

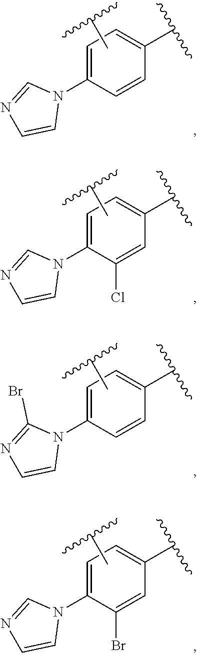

In embodiments, W is substituted, (-L⁵-R⁵)-substituted, (-L¹-R¹)-substituted, (-L²-R²)-substituted, or unsubstituted

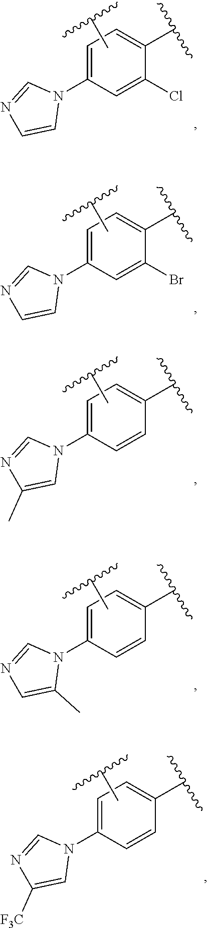

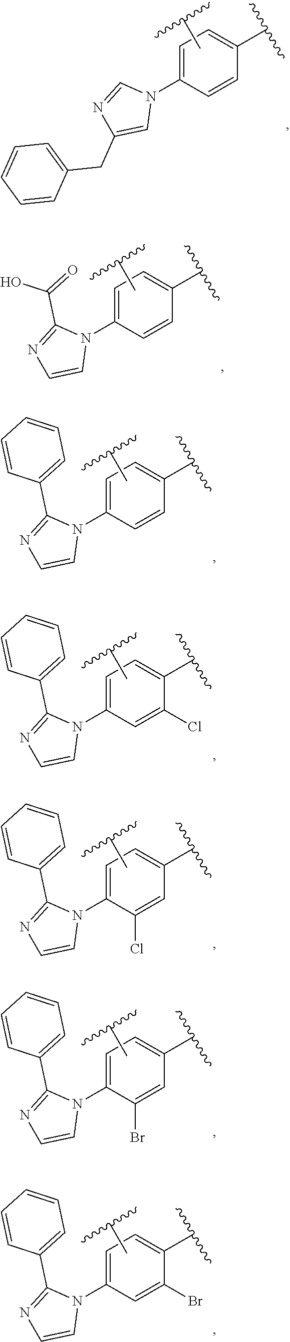

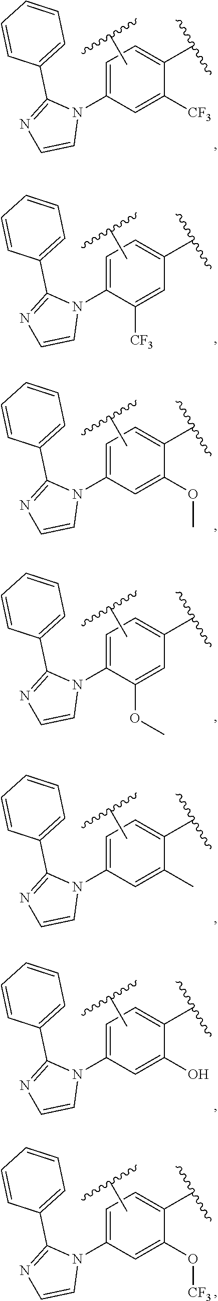

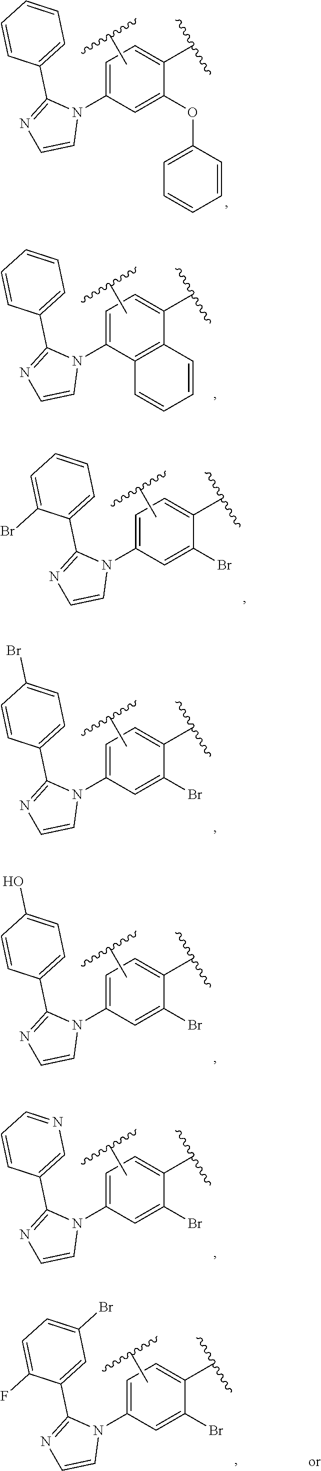

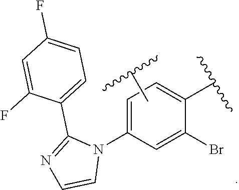

In embodiments, W is substituted, (-L⁵-R⁵)-substituted, (-L¹-R¹)-substituted, (-L²-R²)-substituted, or unsubstituted

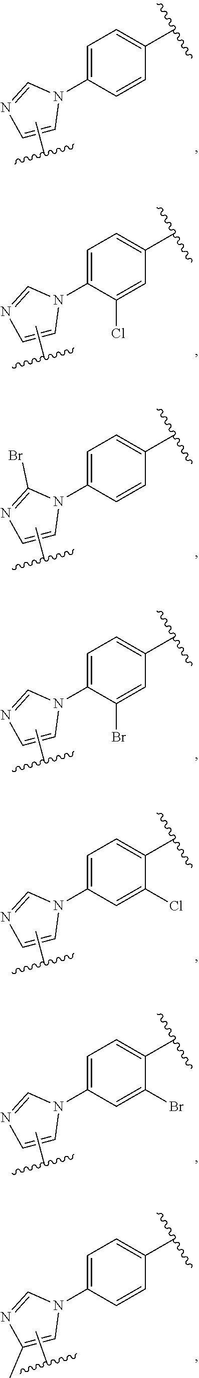

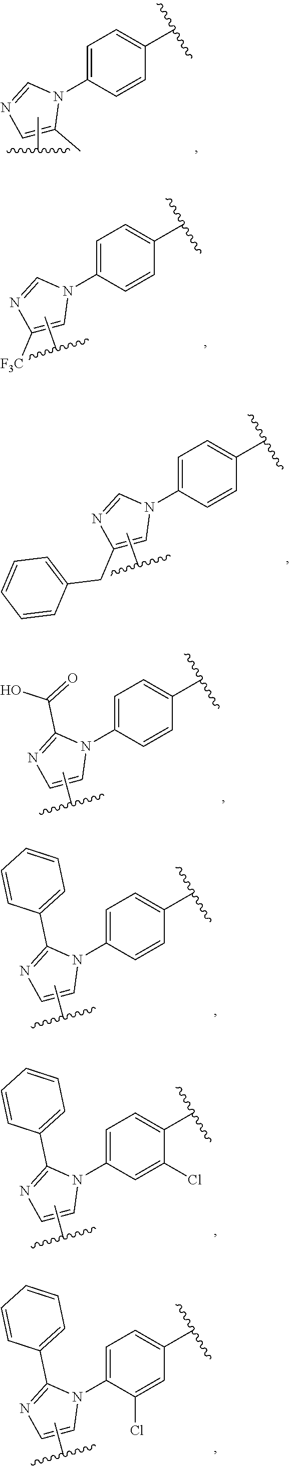

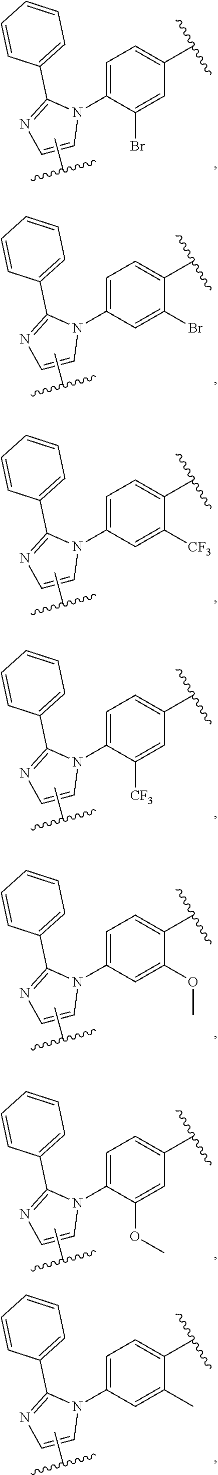

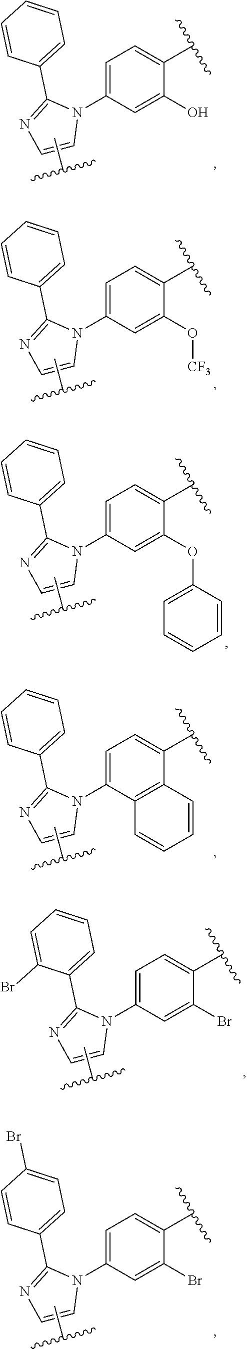

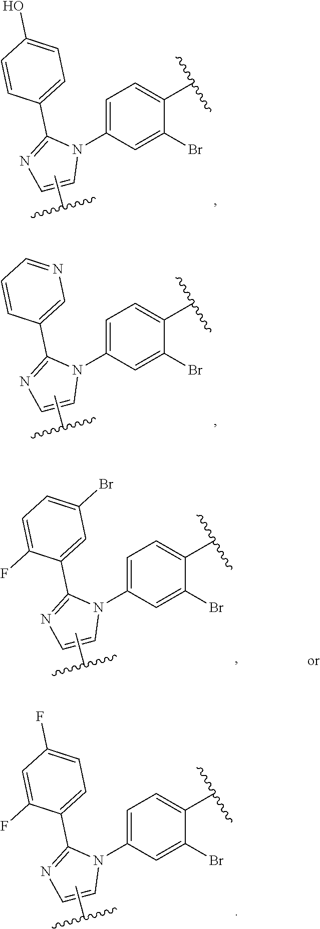

In embodiments, W is substituted, (-L⁵-R⁵)-substituted, (-L¹-R¹)-substituted, (-L²-R²)-substituted, or unsubstituted

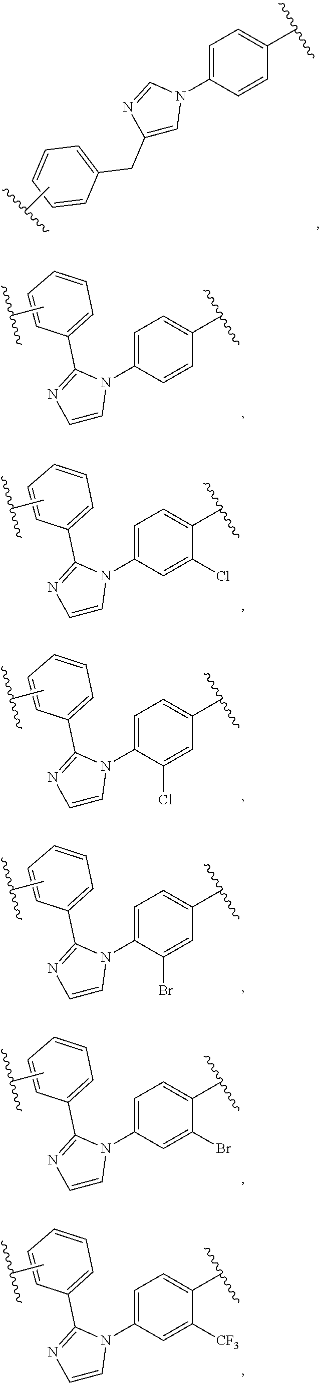

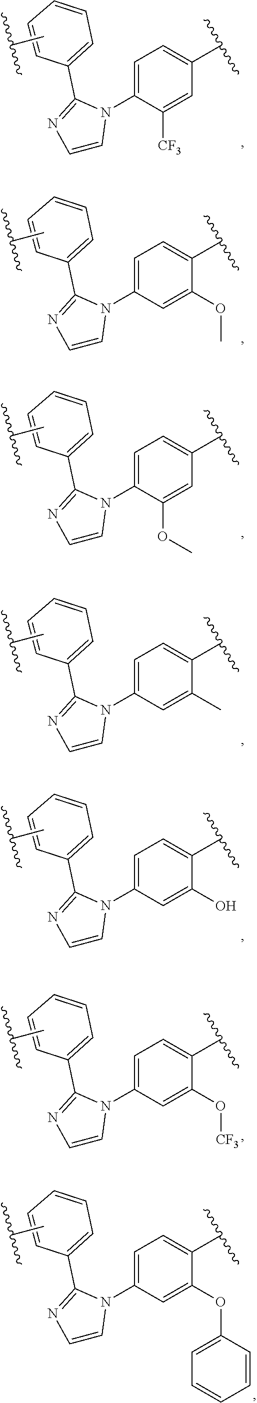

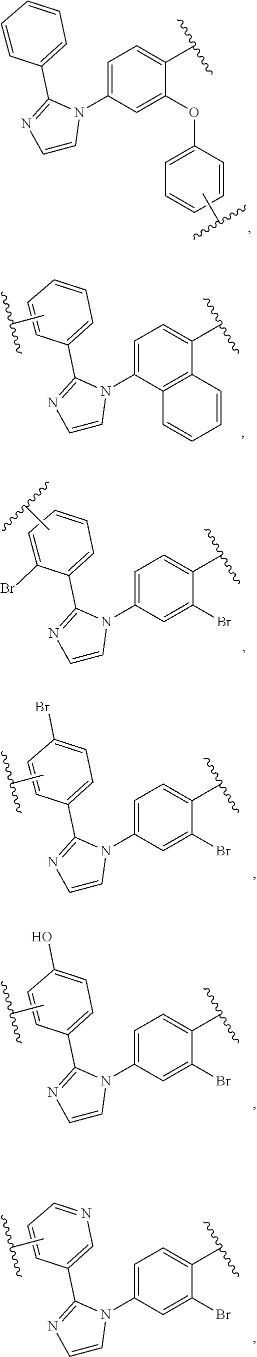

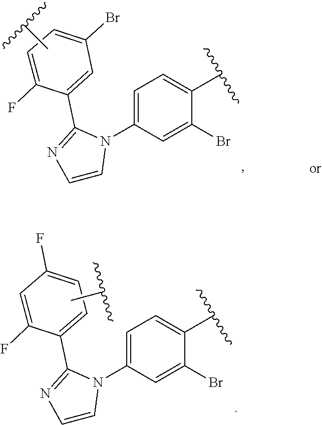

In embodiments, W is substituted, (-L⁵-R⁵)-substituted, (-L¹-R¹)-substituted, (-L²-R²)-substituted, or unsubstituted

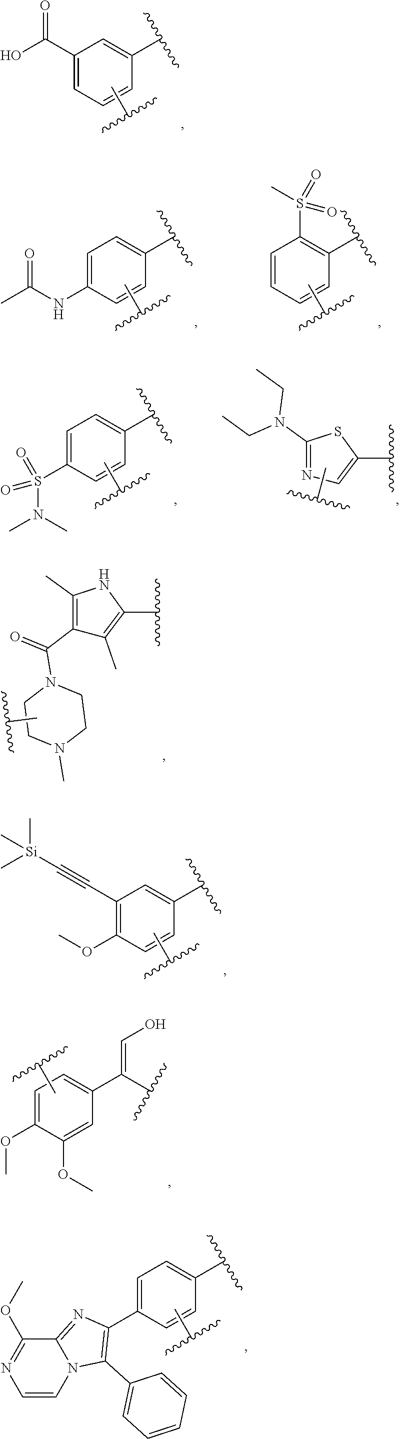

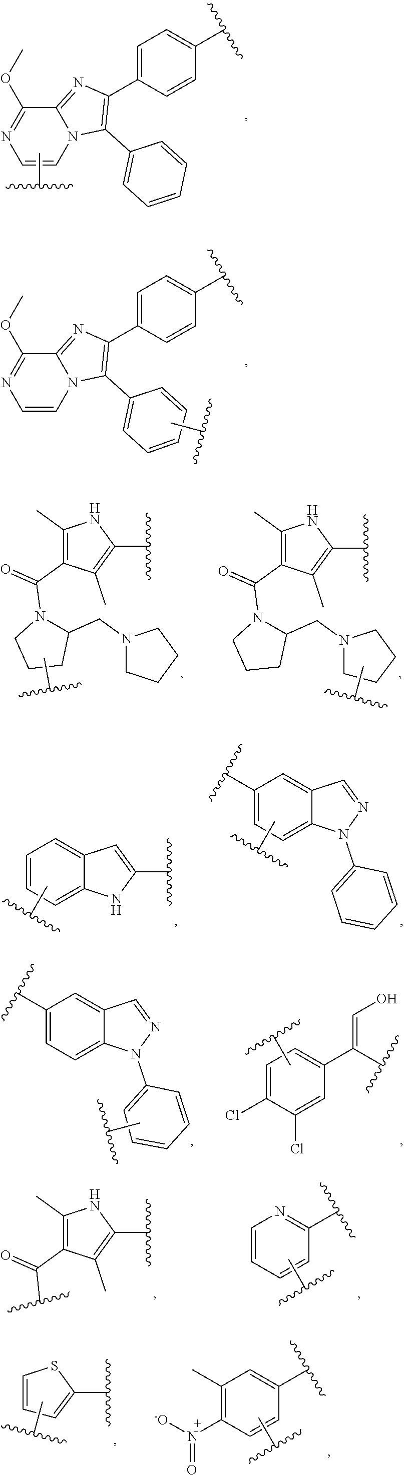

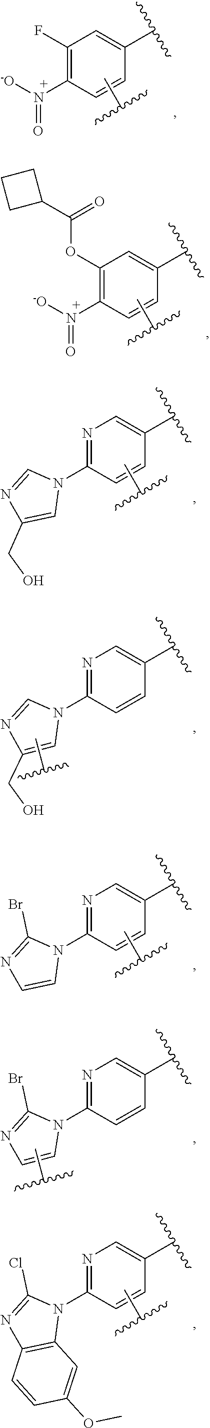

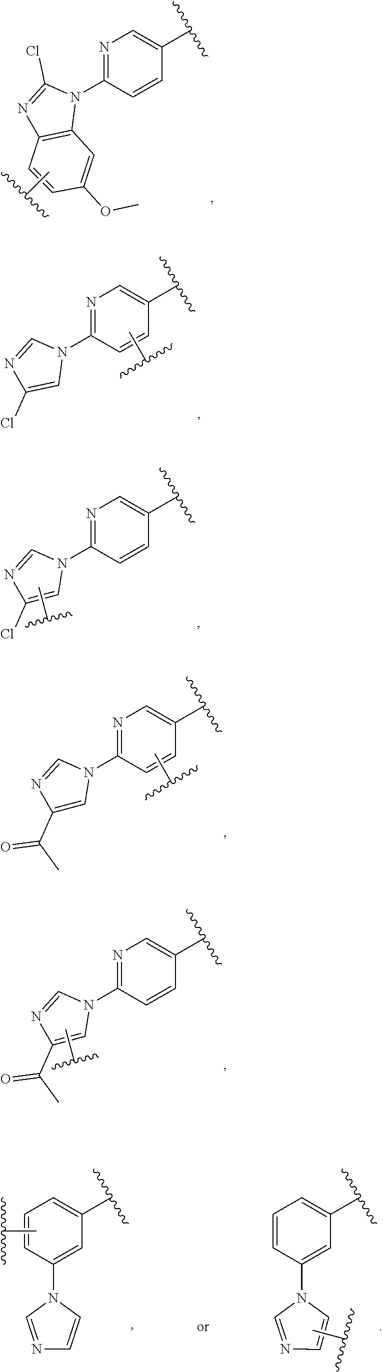

In embodiments, W is substituted, (-L⁵-R⁵)-substituted, (-L¹-R¹)-substituted, (-L²-R²)-substituted, or unsubstituted

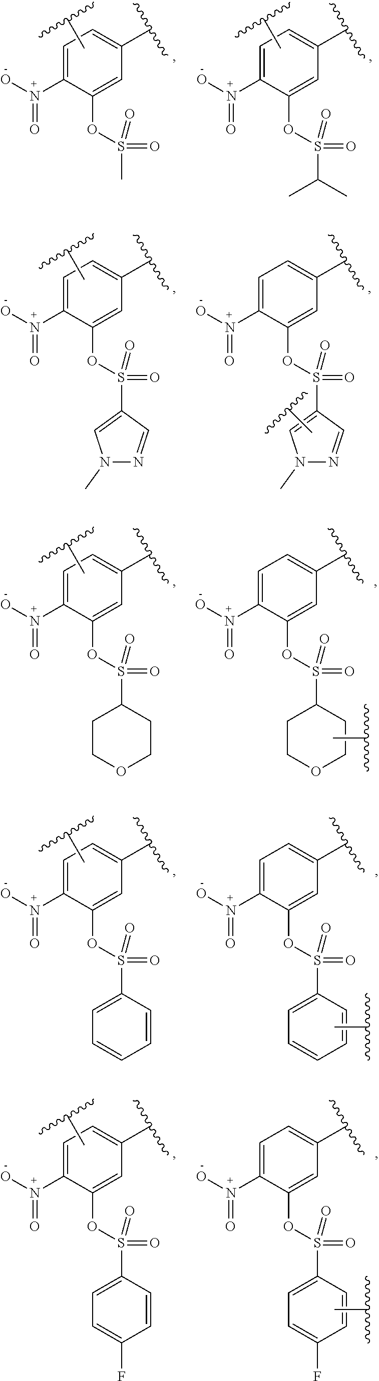

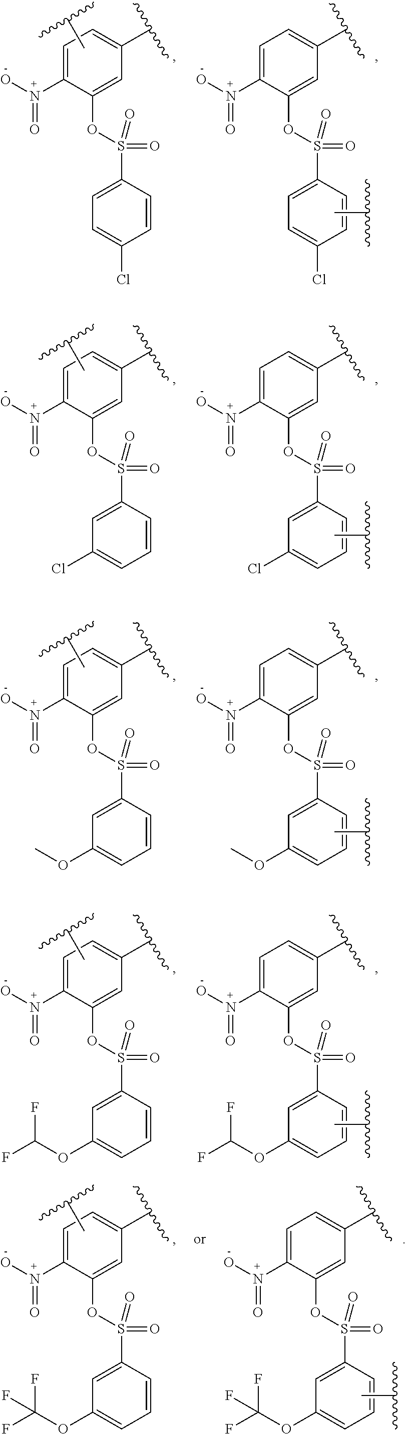

In embodiments, W is substituted, (-L⁵-R⁵)-substituted, (-L¹-R¹)-substituted, (-L²-R²)-substituted, or unsubstituted

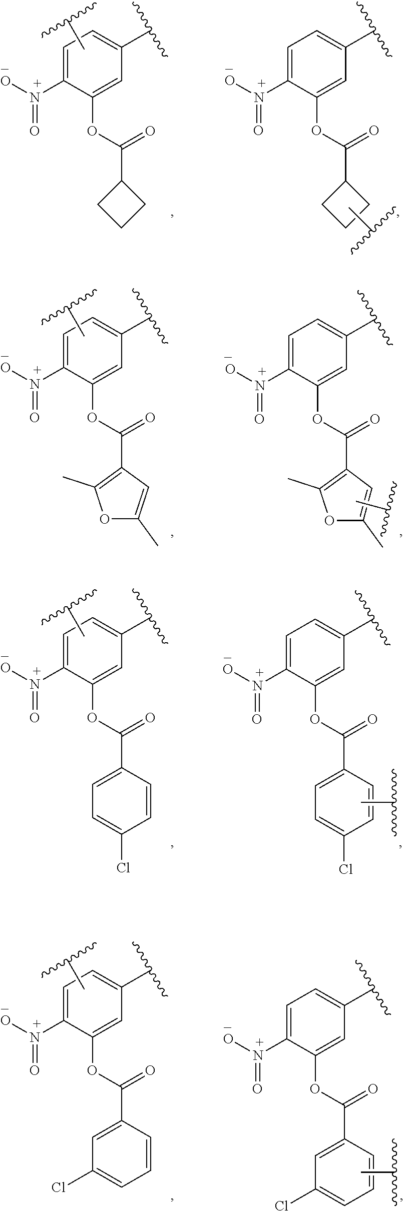

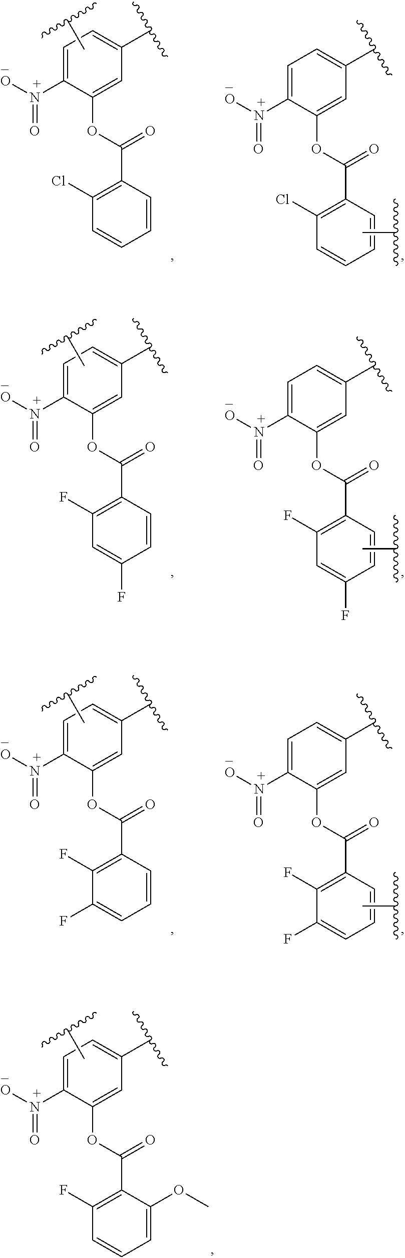

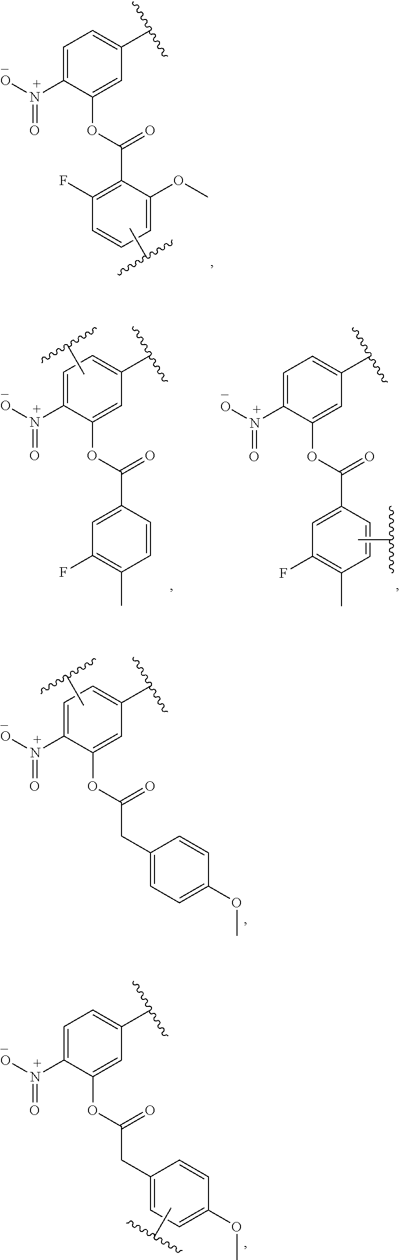

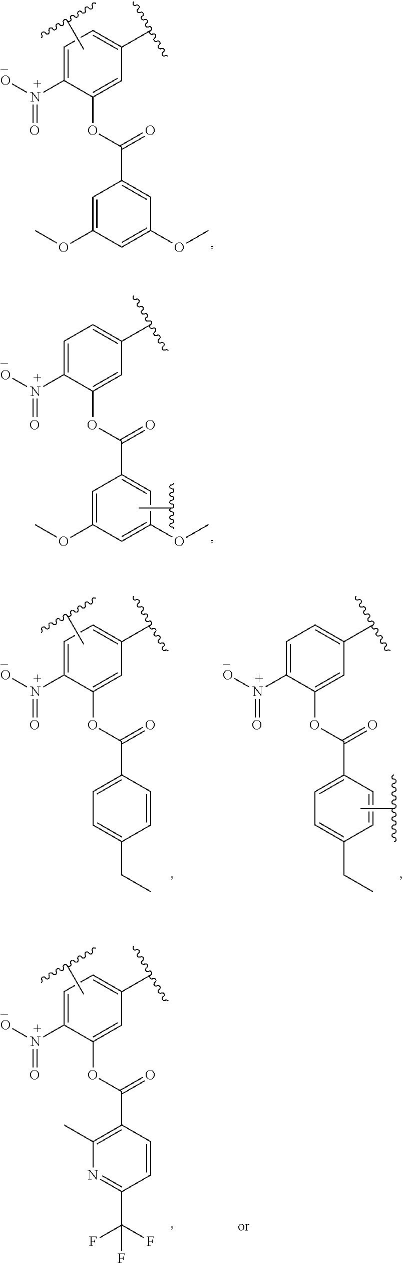

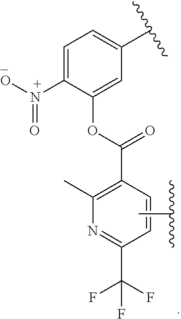

In embodiments, W is substituted, (-L⁵-R⁵)-substituted, (-L¹-R¹)-substituted, (-L²-R²)-substituted, or unsubstituted

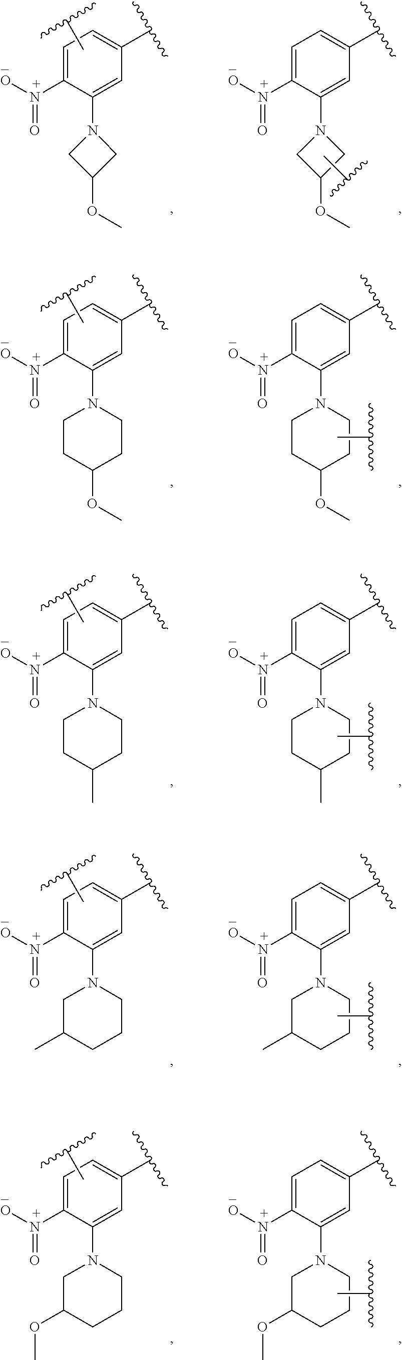

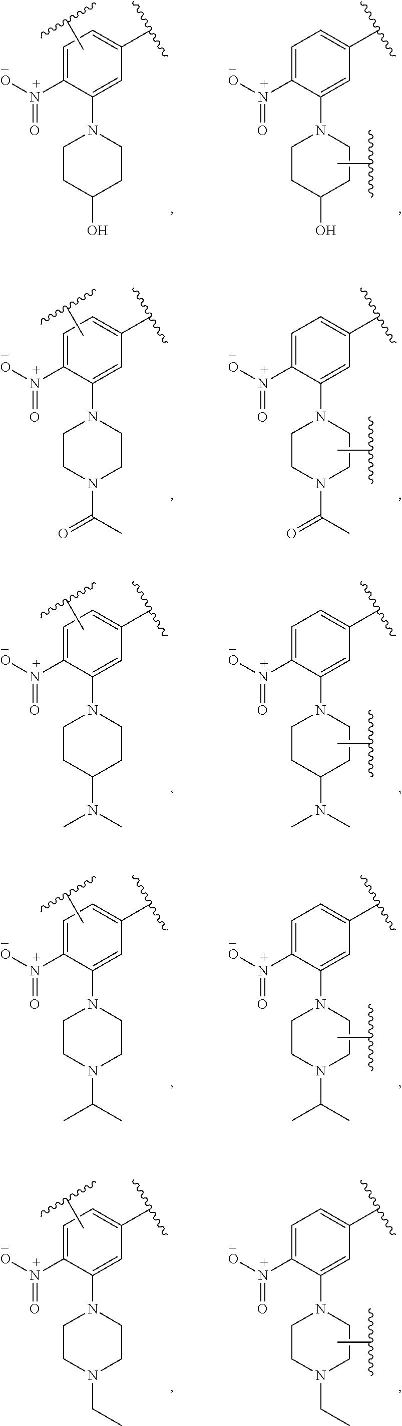

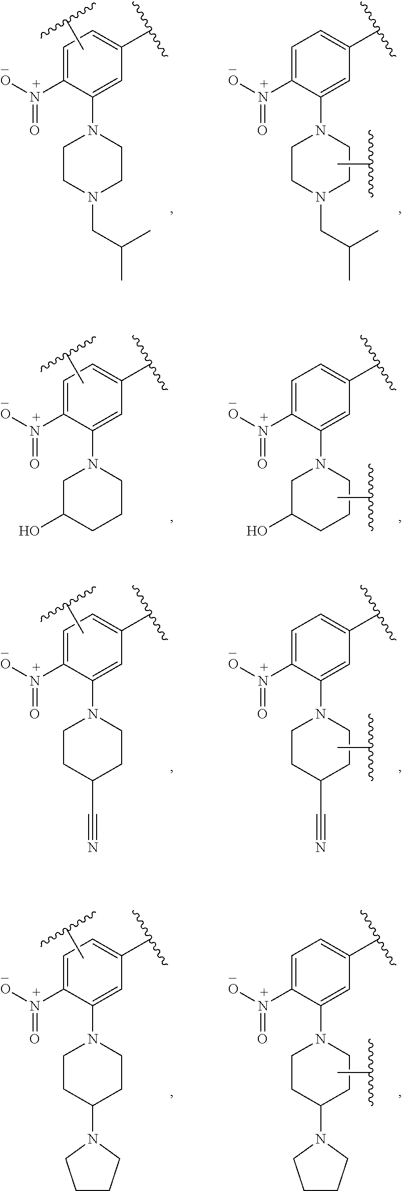

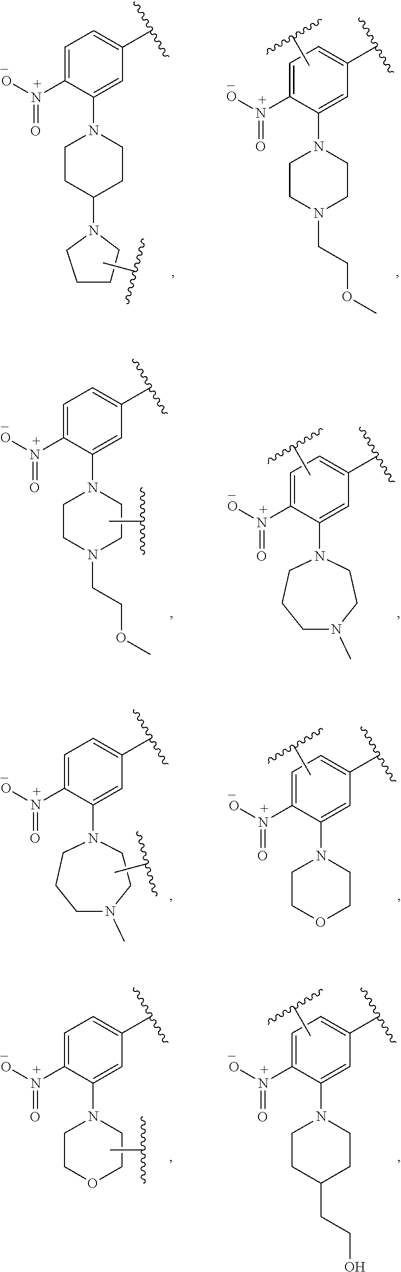

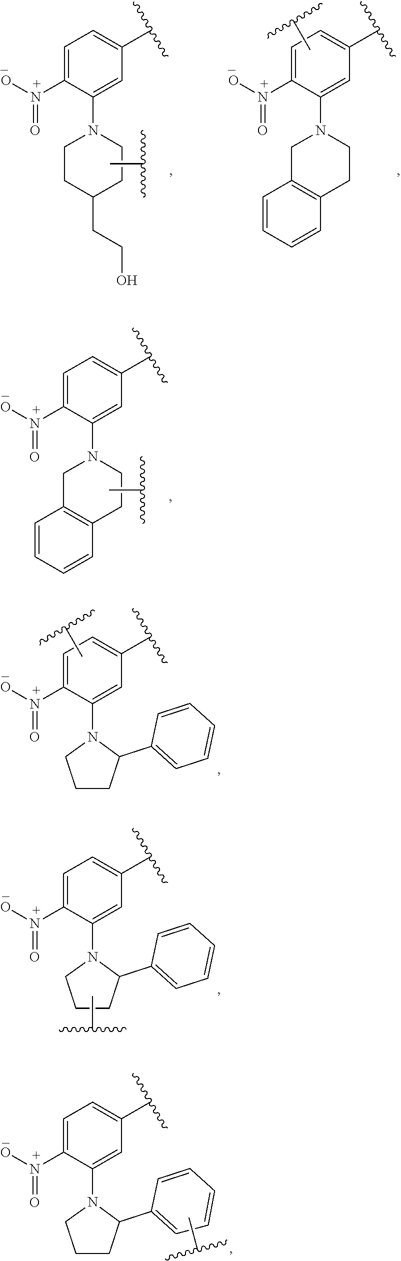

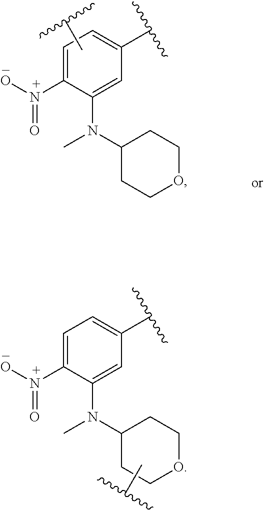

In embodiments, W is substituted, (-L⁵-R⁵)-substituted, (-L¹-R¹)-substituted, (-L²-R²)-substituted, or unsubstituted

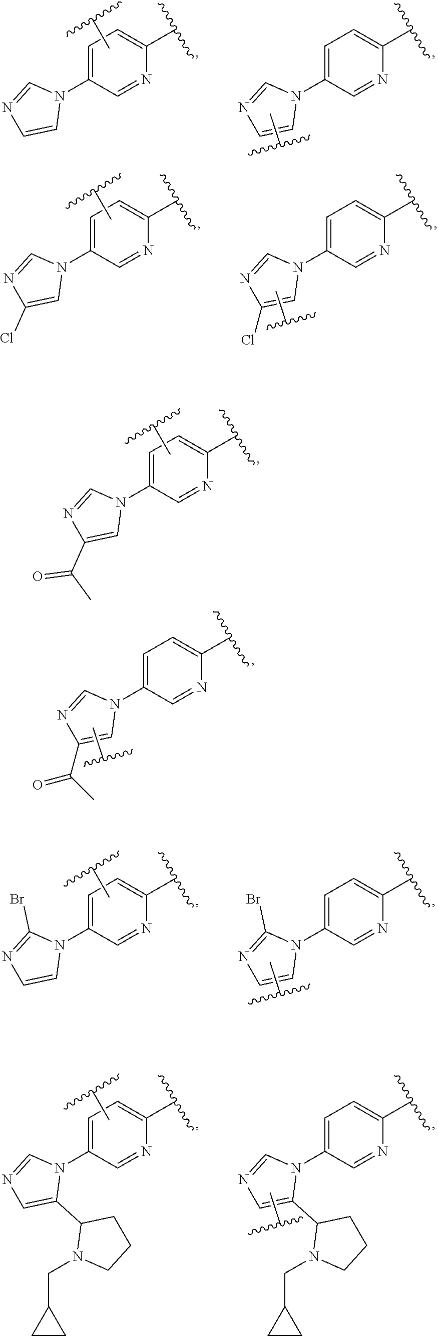

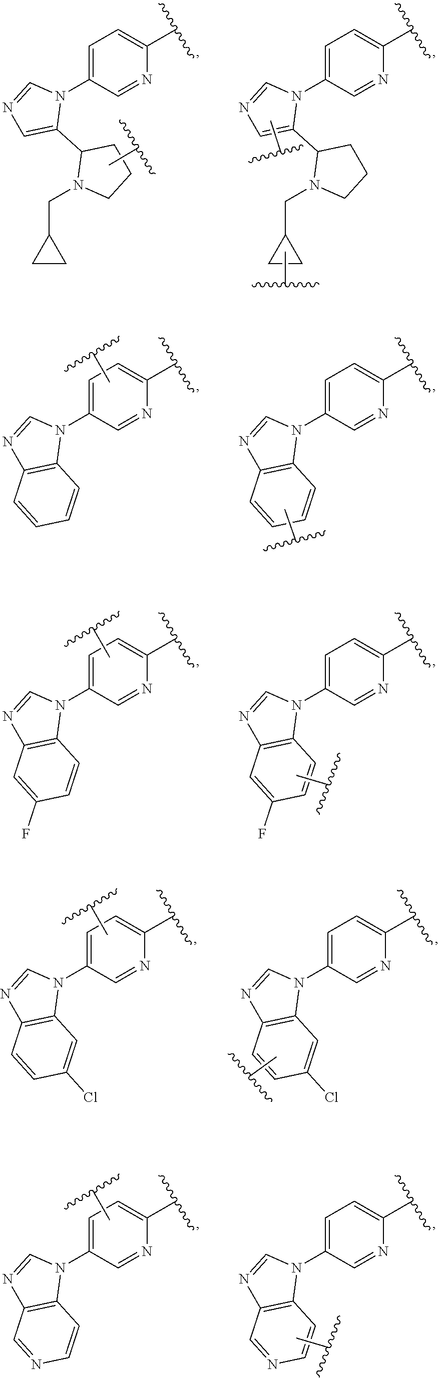

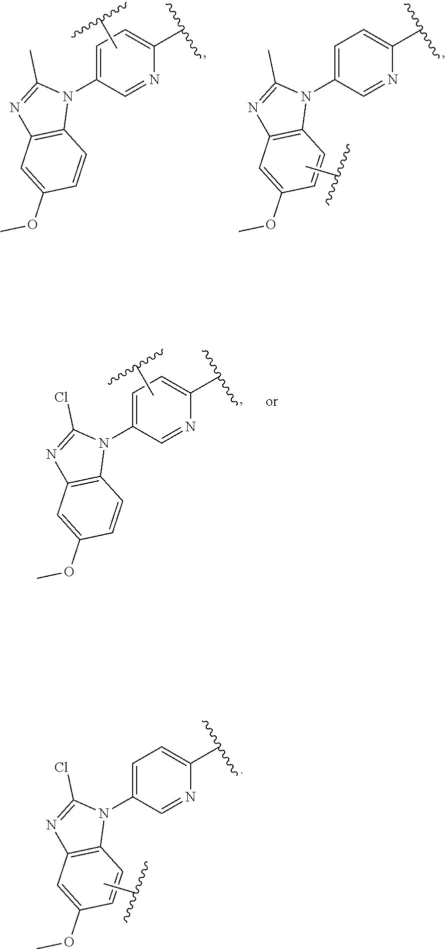

In embodiments, W is substituted, (-L⁵-R⁵)-substituted, (-L¹-R¹)-substituted, (-L²-R²)-substituted, or unsubstituted

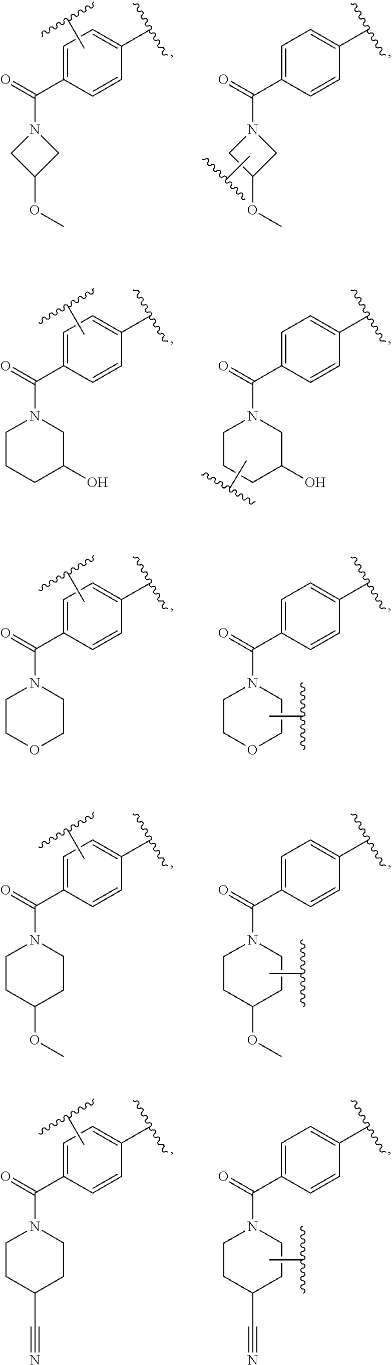

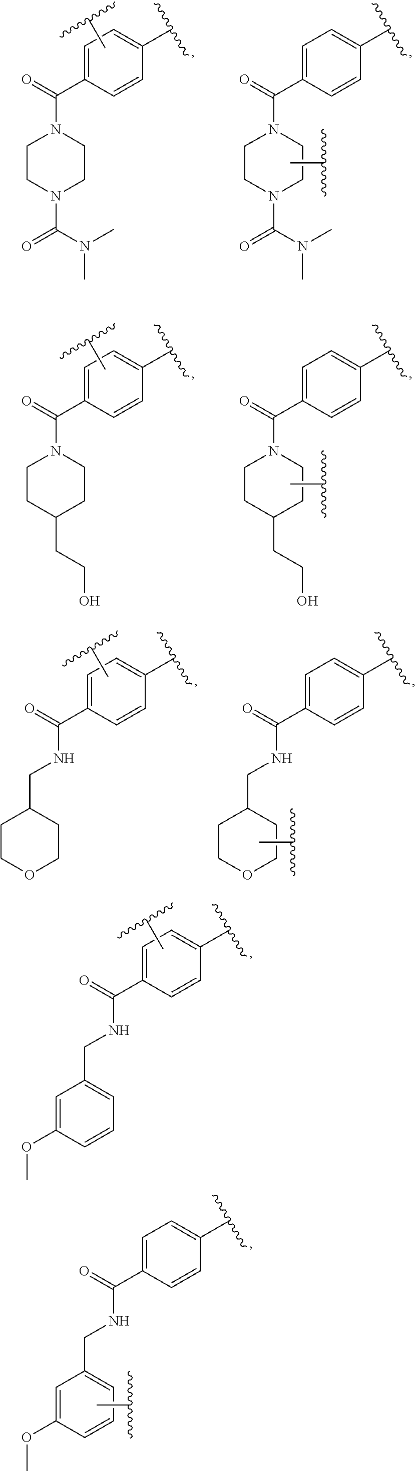

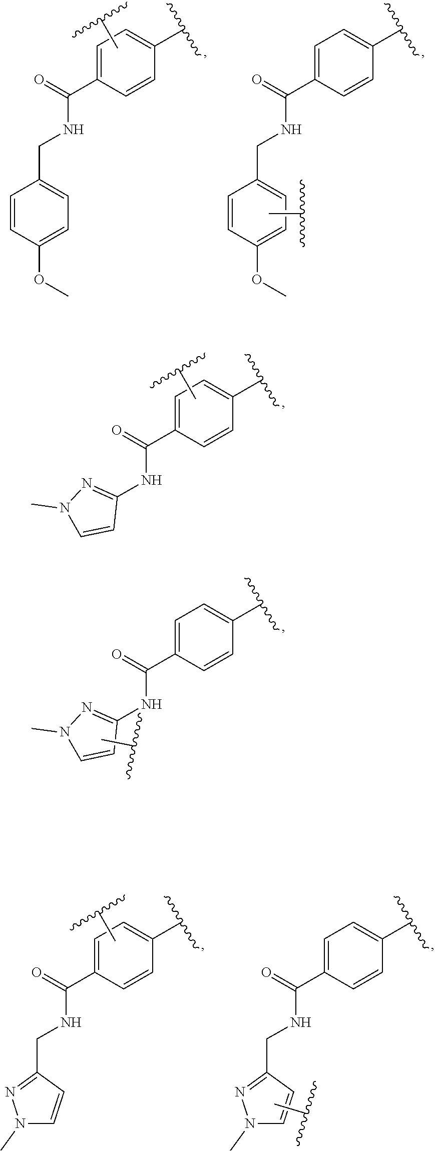

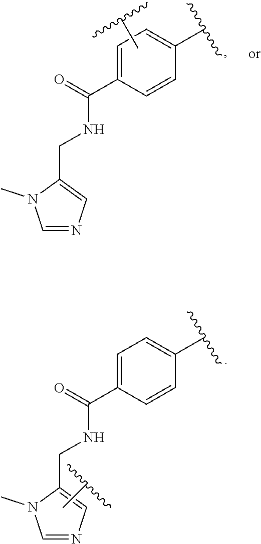

In embodiments, W is substituted, (-L⁵-R⁵)-substituted, (-L¹-R¹)-substituted, (-L²-R²)-substituted, or unsubstituted

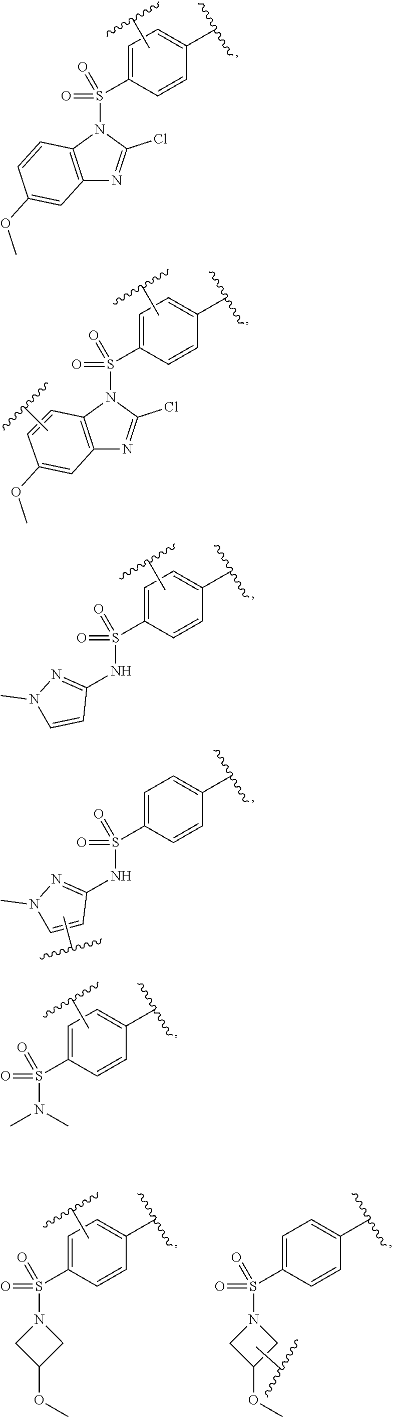

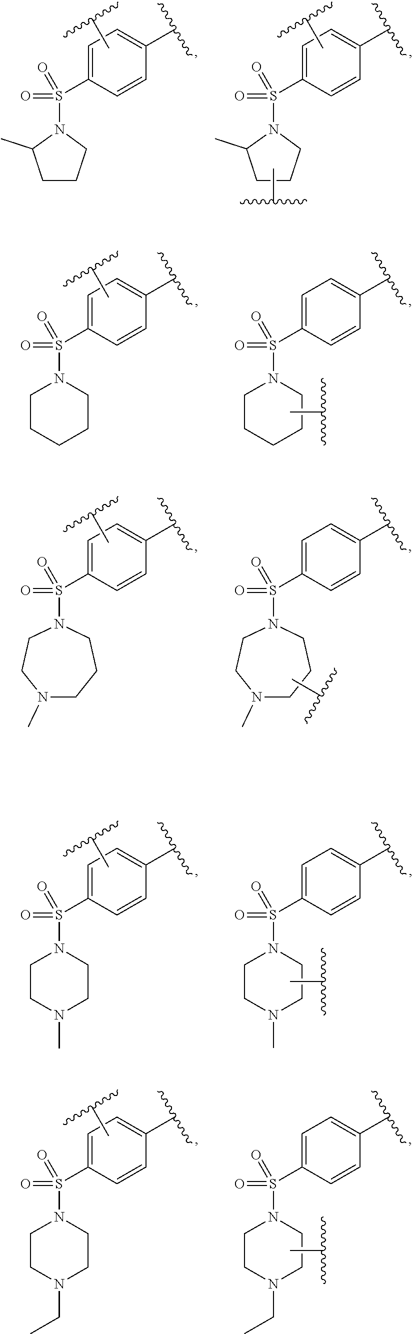

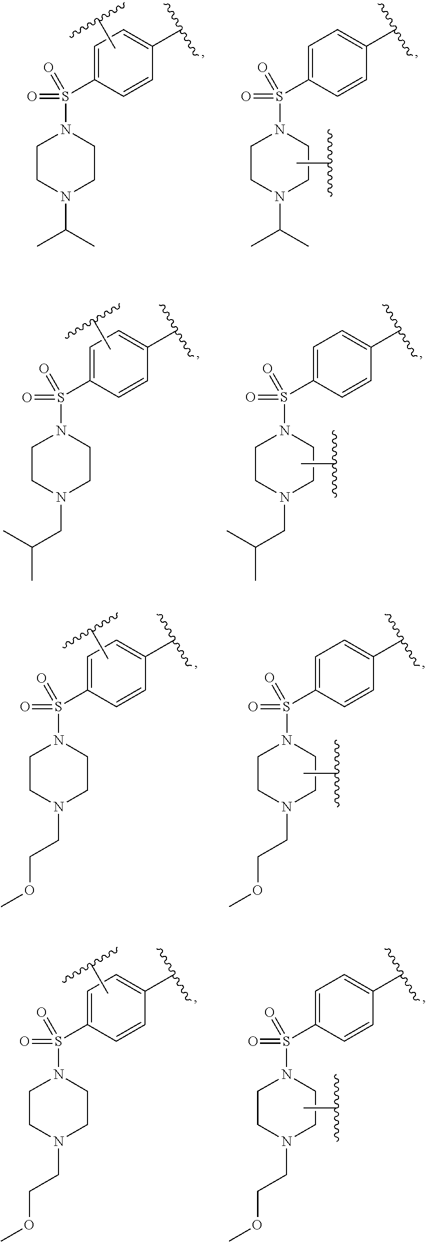

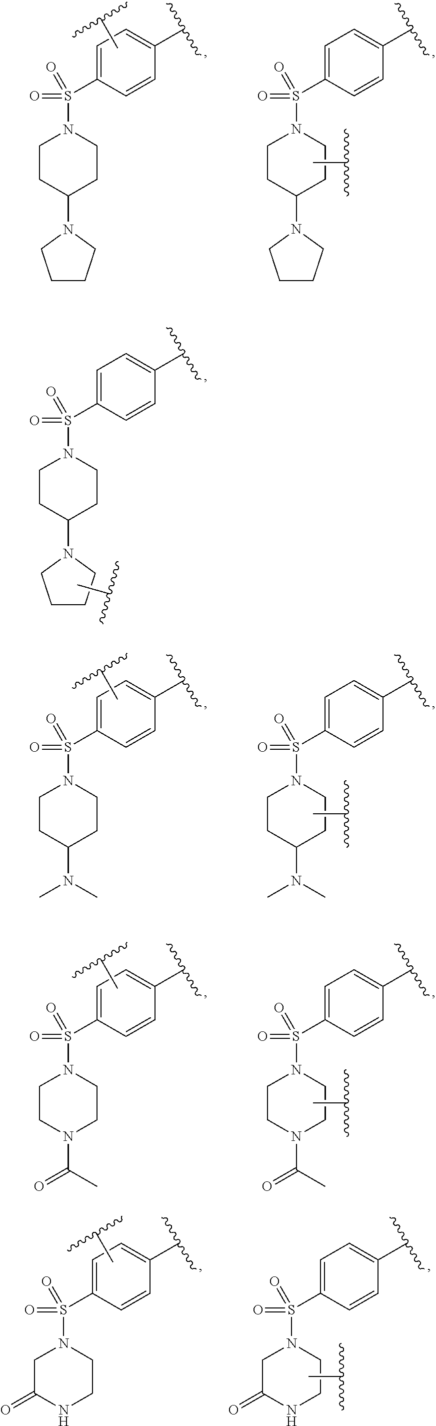

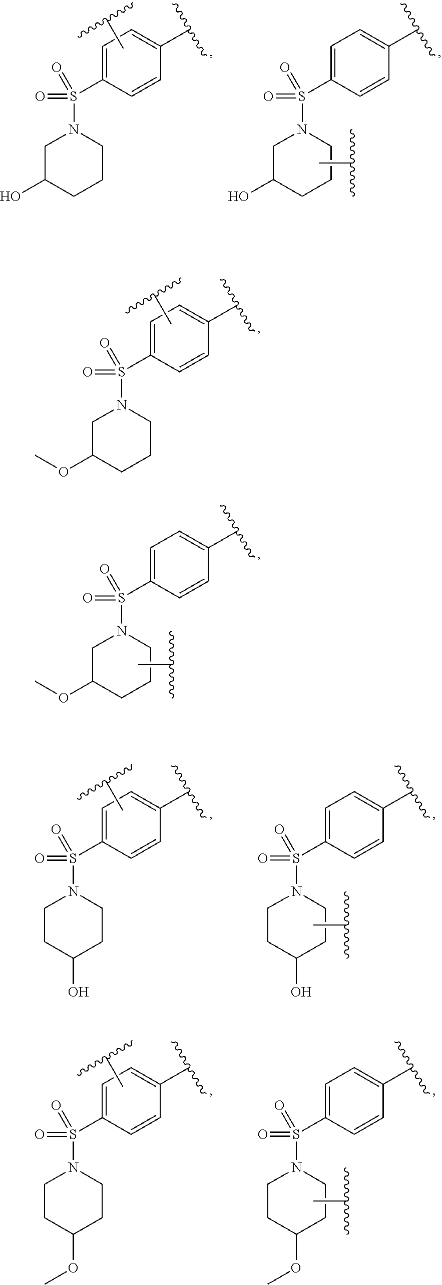

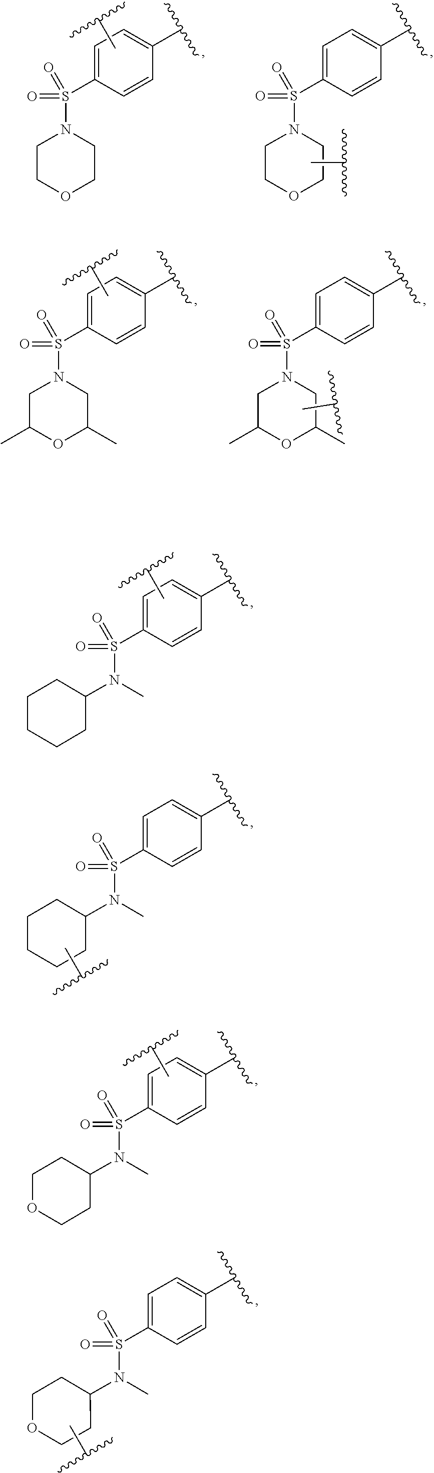

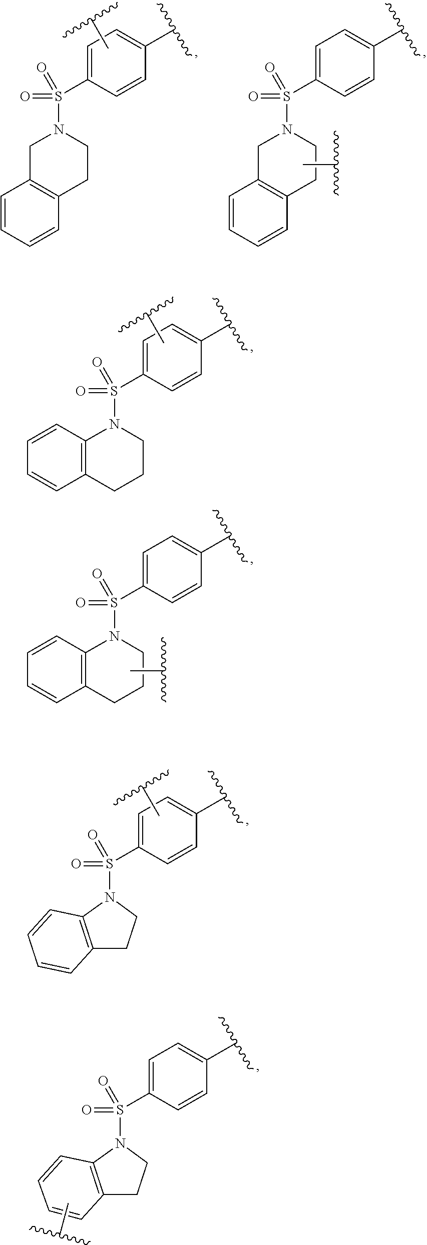

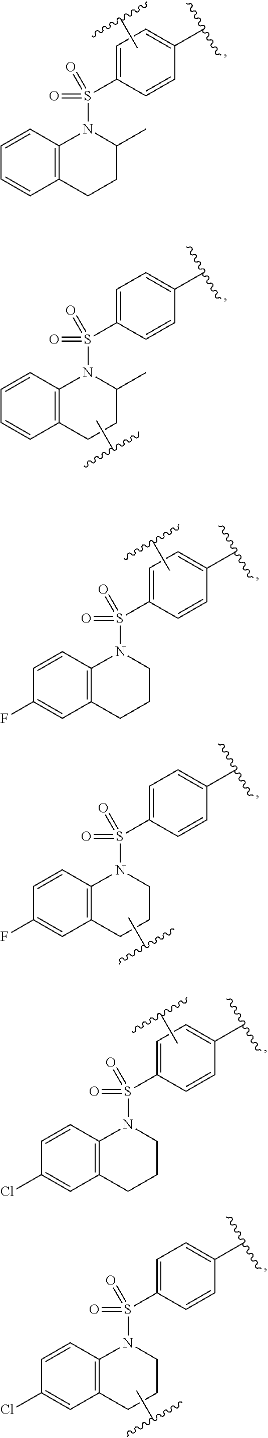

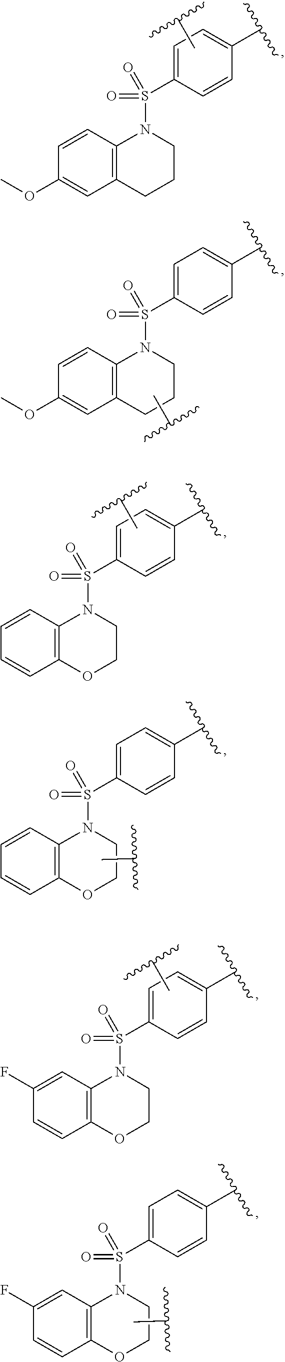

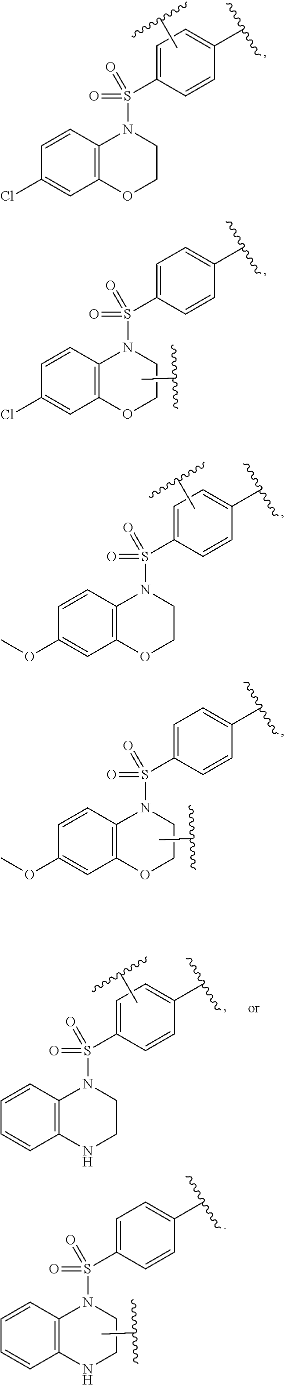

In embodiments, W is substituted, (-L⁵-R⁵)-substituted, (-L¹-R¹)-substituted, (-L²-R²)-substituted, or unsubstituted

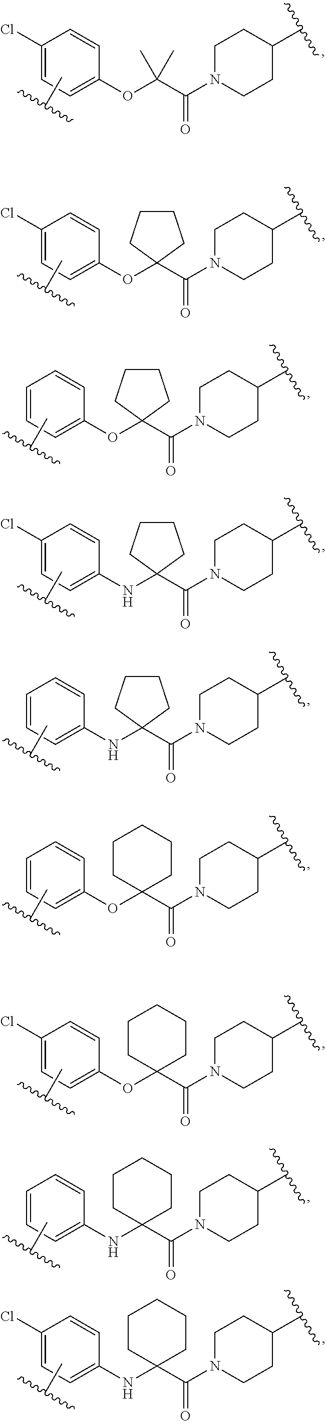

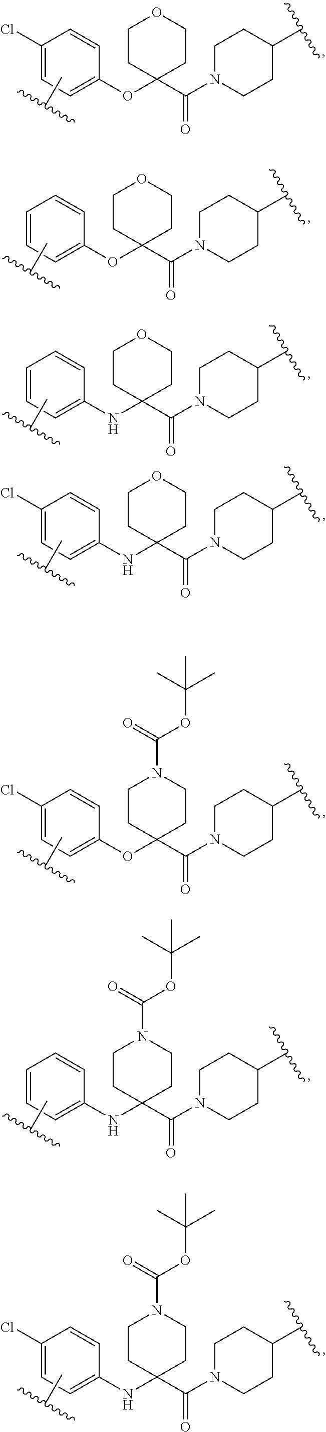

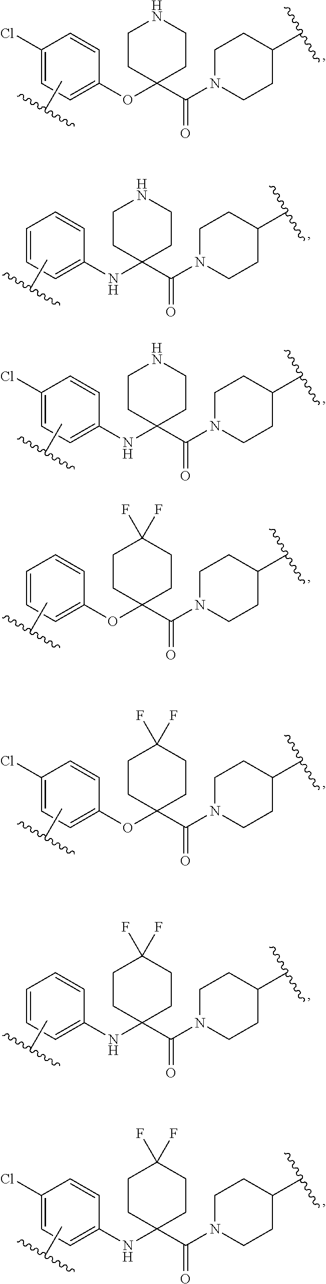

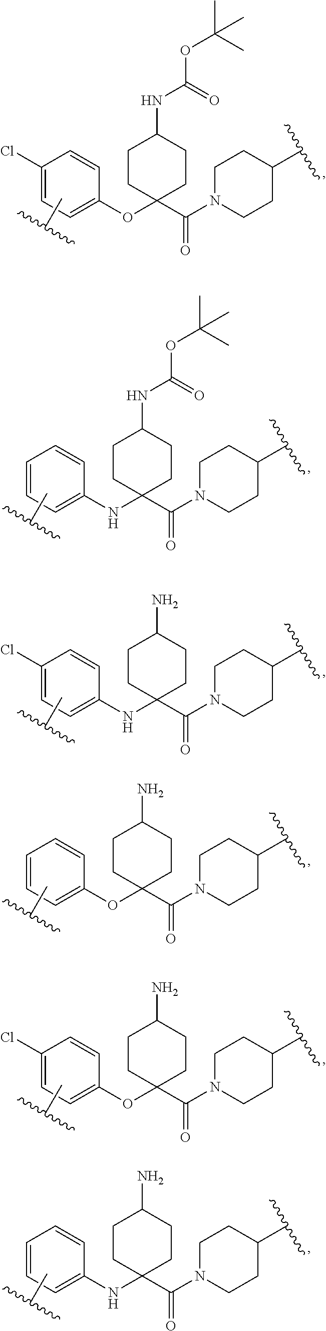

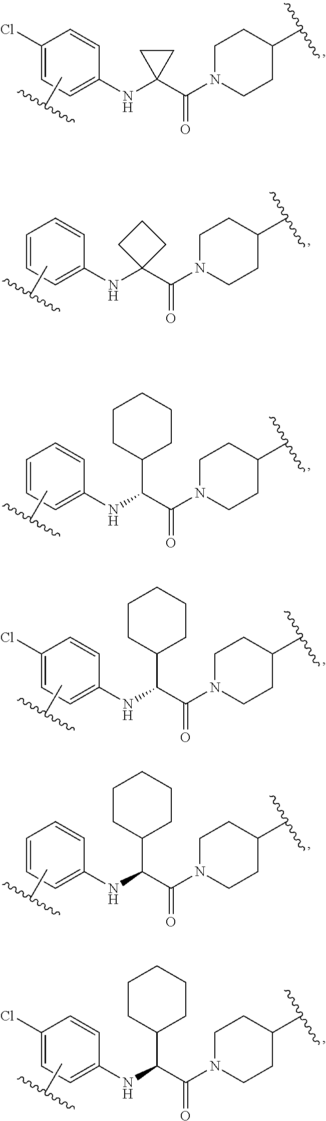

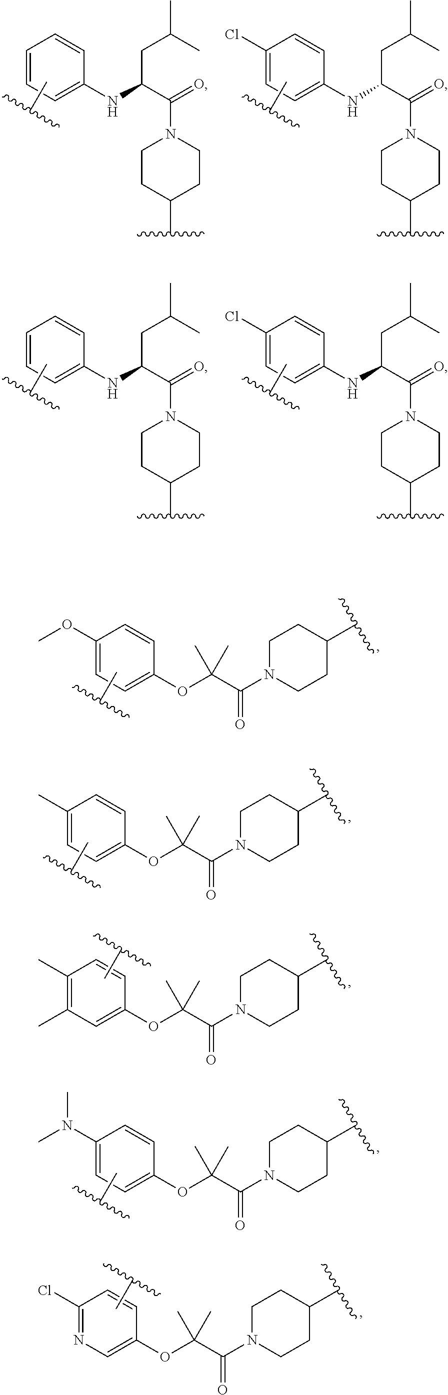

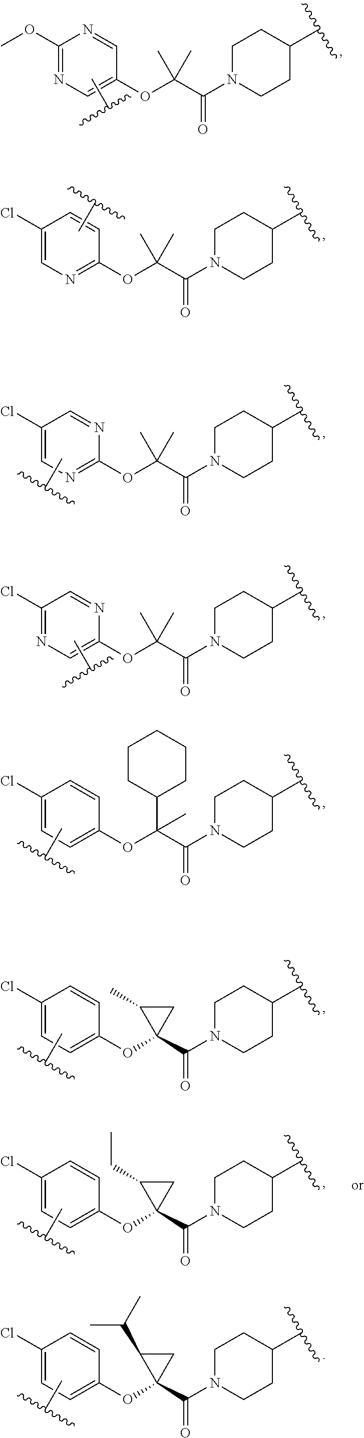

In embodiments, W is substituted, (-L⁵-R⁵)-substituted, (-L¹-R¹)-substituted, (-L²-R²)-substituted, or unsubstituted

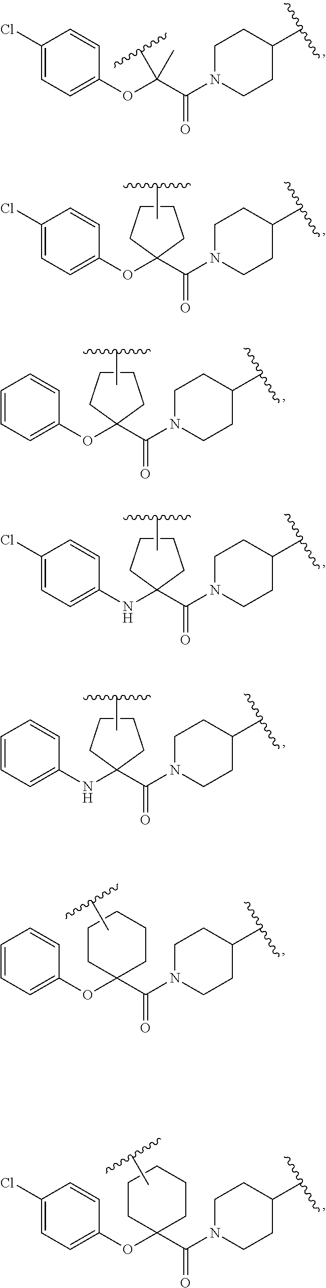

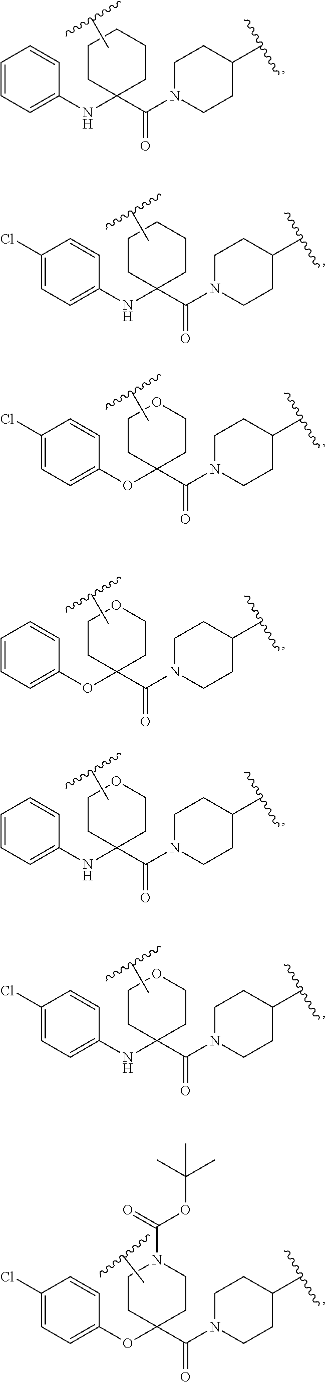

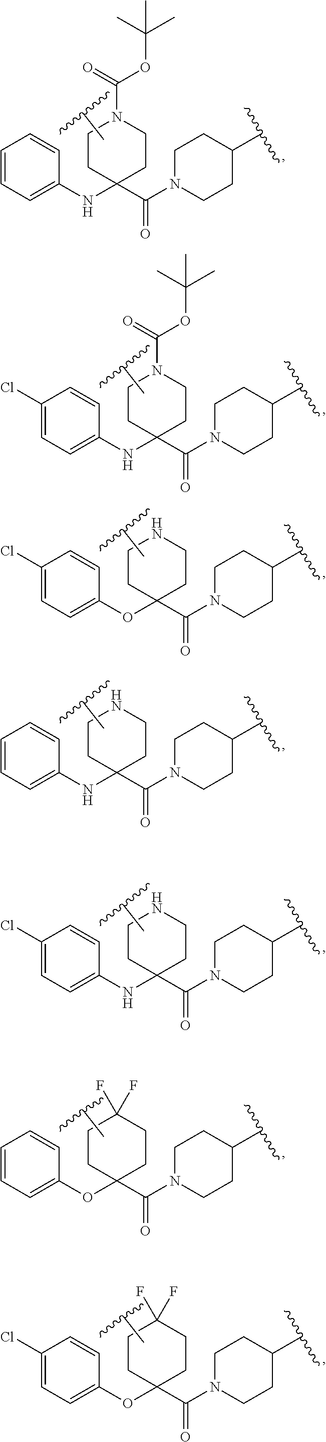

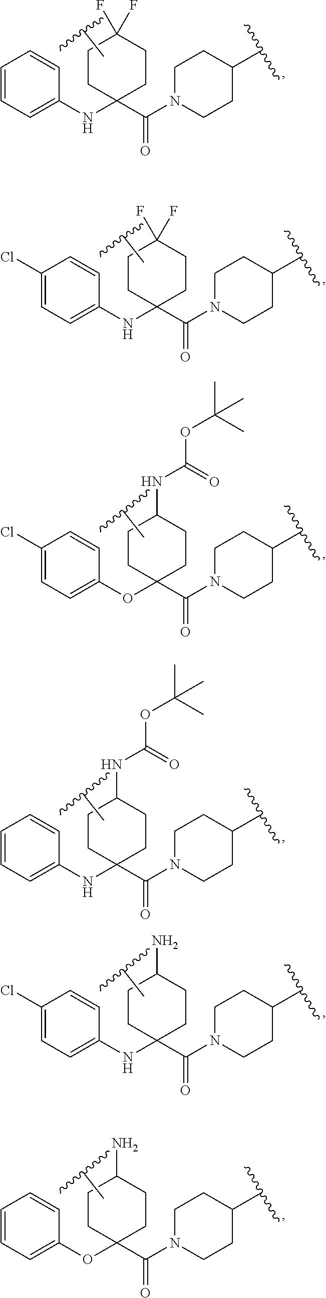

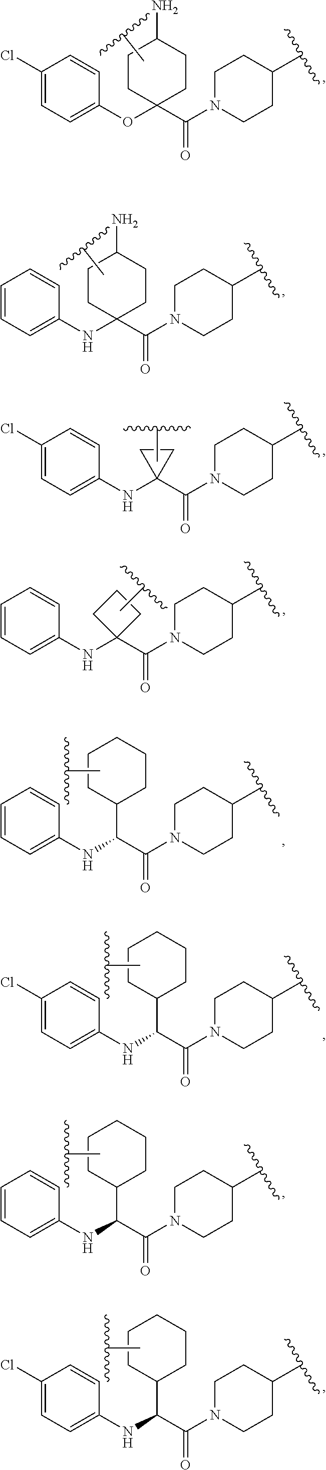

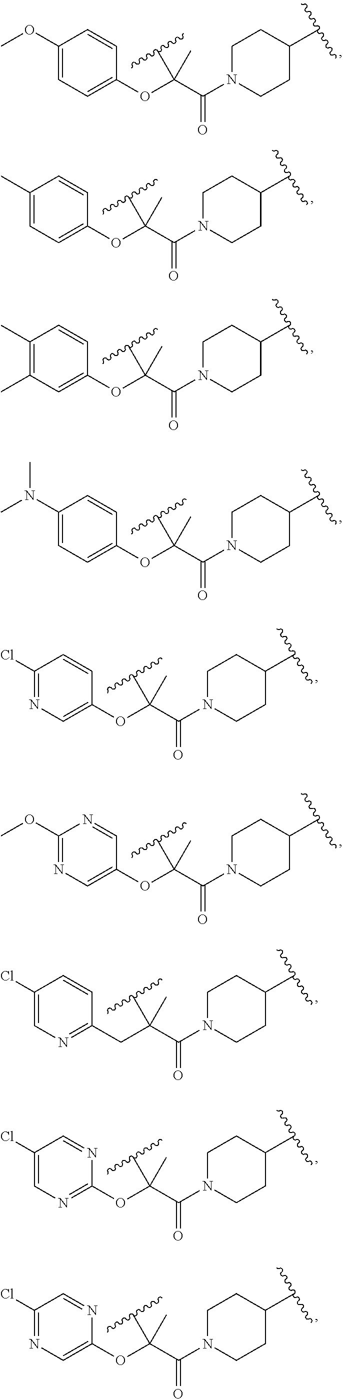

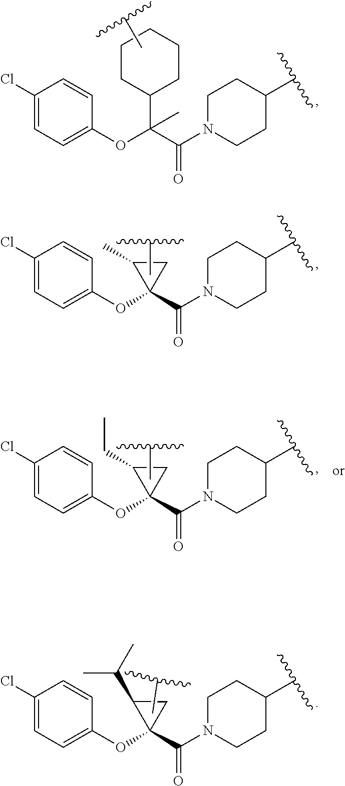

In embodiments, L¹ is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenyl), or substituted or unsubstituted heteroarylene (e.g., 5 to 12 membered, 5 to 10 membered, 5 to 9 membered, or 5 to 6 membered). In embodiments, L¹ is independently a bond. In embodiments, L¹ is independently —S(O)₂—. In embodiments, L¹ is independently —NH—. In embodiments, L¹ is independently —O—. In embodiments, L¹ is independently —S—. In embodiments, L¹ is independently —C(O)—. In embodiments, L¹ is independently —NHS(O)₂—. In embodiments, L¹ is independently —S(O)₂NH—. In embodiments, L¹ is independently —C(O)NH—. In embodiments, L¹ is independently —NHC(O)—. In embodiments, L¹ is independently —NHC(O)NH—. In embodiments, L¹ is independently —C(O)O—. In embodiments, L¹ is independently —OC(O)—. In embodiments, L¹ is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is independently substituted or unsubstituted alkylene. In embodiments, L¹ is independently unsubstituted alkylene. In embodiments, L¹ is independently unsubstituted methylene. In embodiments, L¹ is independently unsubstituted ethylene. In embodiments, L¹ is independently unsubstituted propylene. In embodiments, L¹ is independently substituted or unsubstituted heteroalkylene. In embodiments, L¹ is independently unsubstituted heteroalkylene. In embodiments, L¹ is independently substituted or unsubstituted cycloalkylene. In embodiments, L¹ is independently unsubstituted cycloalkylene. In embodiments, L¹ is independently substituted or unsubstituted heterocycloalkylene. In embodiments, L¹ is independently unsubstituted heterocycloalkylene. In embodiments, L¹ is independently substituted or unsubstituted arylene. In embodiments, L¹ is independently unsubstituted phenylene. In embodiments, L¹ is independently substituted or unsubstituted heteroarylene. In embodiments, L¹ is independently unsubstituted heteroarylene. In embodiments, L¹ is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L¹ is independently substituted or unsubstituted C₁-C₆ alkylene. In embodiments, L¹ is independently unsubstituted C₁-C₆ alkylene. In embodiments, L¹ is independently unsubstituted methylene. In embodiments, L¹ is independently unsubstituted ethylene. In embodiments, L¹ is independently unsubstituted propylene. In embodiments, L¹ is independently substituted or unsubstituted 2 to 6 membered heteroalkylene. In embodiments, L¹ is independently unsubstituted 2 to 6 membered heteroalkylene. In embodiments, L¹ is independently substituted or unsubstituted C₃-C₆ cycloalkylene. In embodiments, L¹ is independently unsubstituted C₃-C₆ cycloalkylene. In embodiments, L¹ is independently substituted or unsubstituted 3 to 6 membered heterocycloalkylene. In embodiments, L¹ is independently unsubstituted 3 to 6 membered heterocycloalkylene. In embodiments, L¹ is independently substituted or unsubstituted C₆-C₁₀ arylene. In embodiments, L¹ is independently unsubstituted C₆-C₁₀ arylene. In embodiments, L¹ is independently substituted phenylene. In embodiments, L¹ is independently unsubstituted phenylene. In embodiments, L¹ is independently substituted or unsubstituted 5 to 10 membered heteroarylene. In embodiments, L¹ is independently substituted or unsubstituted 5 to 6 membered heteroarylene. In embodiments, L¹ is independently unsubstituted 5 to 10 membered heteroarylene. In embodiments, L¹ is independently unsubstituted 5 to 6 membered heteroarylene.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L² is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered). In embodiments, L² is independently a bond. In embodiments, L² is independently —S(O)₂—. In embodiments, L² is independently —NH—. In embodiments, L² is independently —O—. In embodiments, L² is independently —S—. In embodiments, L² is independently —C(O)—. In embodiments, L² is independently —NHS(O)₂—. In embodiments, L² is independently —S(O)₂NH—. In embodiments, L² is independently —C(O)NH—. In embodiments, L² is independently —NHC(O)—. In embodiments, L² is independently —NHC(O)NH—. In embodiments, L² is independently —C(O)O—. In embodiments, L² is independently —OC(O)—. In embodiments, L² is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is independently substituted or unsubstituted alkylene. In embodiments, L² is independently unsubstituted alkylene. In embodiments, L² is independently unsubstituted methylene. In embodiments, L² is independently unsubstituted ethylene. In embodiments, L² is independently unsubstituted propylene. In embodiments, L² is independently substituted or unsubstituted heteroalkylene. In embodiments, L² is independently unsubstituted heteroalkylene. In embodiments, L² is independently substituted or unsubstituted cycloalkylene. In embodiments, L² is independently unsubstituted cycloalkylene. In embodiments, L² is independently substituted or unsubstituted heterocycloalkylene. In embodiments, L² is independently unsubstituted heterocycloalkylene. In embodiments, L² is independently substituted or unsubstituted arylene. In embodiments, L² is independently unsubstituted phenylene. In embodiments, L² is independently substituted or unsubstituted heteroarylene. In embodiments, L² is independently unsubstituted heteroarylene. In embodiments, L² is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L² is independently substituted or unsubstituted C₁-C₆ alkylene. In embodiments, L² is independently unsubstituted C₁-C₆ alkylene. In embodiments, L² is independently unsubstituted methylene. In embodiments, L² is independently unsubstituted ethylene. In embodiments, L² is independently unsubstituted propylene. In embodiments, L² is independently substituted or unsubstituted 2 to 6 membered heteroalkylene. In embodiments, L² is independently unsubstituted 2 to 6 membered heteroalkylene. In embodiments, L² is independently substituted or unsubstituted C₃-C₆ cycloalkylene. In embodiments, L² is independently unsubstituted C₃-C₆ cycloalkylene. In embodiments, L² is independently substituted or unsubstituted 3 to 6 membered heterocycloalkylene. In embodiments, L² is independently unsubstituted 3 to 6 membered heterocycloalkylene. In embodiments, L² is independently substituted or unsubstituted C₆-C₁₀ arylene. In embodiments, L² is independently unsubstituted C₆-C₁₀ arylene. In embodiments, L² is independently substituted phenylene. In embodiments, L² is independently unsubstituted phenylene. In embodiments, L² is independently substituted or unsubstituted 5 to 10 membered heteroarylene. In embodiments, L² is independently substituted or unsubstituted 5 to 6 membered heteroarylene. In embodiments, L² is independently unsubstituted 5 to 10 membered heteroarylene. In embodiments, L² is independently unsubstituted 5 to 6 membered heteroarylene.

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L³ is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered). In embodiments, L³ is independently a bond. In embodiments, L³ is independently —S(O)₂—. In embodiments, L³ is independently —NH—. In embodiments, L³ is independently —O—. In embodiments, L³ is independently —S—. In embodiments, L³ is independently —C(O)—. In embodiments, L³ is independently —NHS(O)₂—. In embodiments, L³ is independently —S(O)₂NH—. In embodiments, L³ is independently —C(O)NH—. In embodiments, L³ is independently —NHC(O)—. In embodiments, L³ is independently —NHC(O)NH—. In embodiments, L³ is independently —C(O)O—. In embodiments, L³ is independently —OC(O)—. In embodiments, L³ is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is independently substituted or unsubstituted alkylene. In embodiments, L³ is independently unsubstituted alkylene. In embodiments, L³ is independently unsubstituted methylene. In embodiments, L³ is independently unsubstituted ethylene. In embodiments, L³ is independently unsubstituted propylene. In embodiments, L³ is independently substituted or unsubstituted heteroalkylene. In embodiments, L³ is independently unsubstituted heteroalkylene. In embodiments, L³ is independently substituted or unsubstituted cycloalkylene. In embodiments, L³ is independently unsubstituted cycloalkylene. In embodiments, L³ is independently substituted or unsubstituted heterocycloalkylene. In embodiments, L³ is independently unsubstituted heterocycloalkylene. In embodiments, L³ is independently substituted or unsubstituted arylene. In embodiments, L³ is independently unsubstituted phenylene. In embodiments, L³ is independently substituted or unsubstituted heteroarylene. In embodiments, L³ is independently unsubstituted heteroarylene. In embodiments, L³ is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L³ is independently substituted or unsubstituted C₁-C₆ alkylene. In embodiments, L³ is independently unsubstituted C₁-C₆ alkylene. In embodiments, L³ is independently unsubstituted methylene. In embodiments, L³ is independently unsubstituted ethylene. In embodiments, L³ is independently unsubstituted propylene. In embodiments, L³ is independently substituted or unsubstituted 2 to 6 membered heteroalkylene. In embodiments, L³ is independently unsubstituted 2 to 6 membered heteroalkylene. In embodiments, L³ is independently substituted or unsubstituted C₃-C₆ cycloalkylene. In embodiments, L³ is independently unsubstituted C₃-C₆ cycloalkylene. In embodiments, L³ is independently substituted or unsubstituted 3 to 6 membered heterocycloalkylene. In embodiments, L³ is independently unsubstituted 3 to 6 membered heterocycloalkylene. In embodiments, L³ is independently substituted or unsubstituted C₆-C₁₀ arylene. In embodiments, L³ is independently unsubstituted C₆-C₁₀ arylene. In embodiments, L³ is independently substituted phenylene. In embodiments, L³ is independently unsubstituted phenylene. In embodiments, L³ is independently substituted or unsubstituted 5 to 10 membered heteroarylene. In embodiments, L³ is independently substituted or unsubstituted 5 to 6 membered heteroarylene. In embodiments, L³ is independently unsubstituted 5 to 10 membered heteroarylene. In embodiments, L³ is independently unsubstituted 5 to 6 membered heteroarylene.

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L⁵ is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenyl), or substituted or unsubstituted heteroarylene (e.g., 5 to 12 membered, 5 to 10 membered, 5 to 9 membered, or 5 to 6 membered). In embodiments, L⁵ is independently a bond. In embodiments, L⁵ is independently —S(O)₂—. In embodiments, L⁵ is independently —NH—. In embodiments, L⁵ is independently —O—. In embodiments, L⁵ is independently —S—. In embodiments, L⁵ is independently —C(O)—. In embodiments, L⁵ is independently —NHS(O)₂—. In embodiments, L⁵ is independently —S(O)₂NH—. In embodiments, L⁵ is independently —C(O)NH—. In embodiments, L⁵ is independently —NHC(O)—. In embodiments, L⁵ is independently —NHC(O)NH—. In embodiments, L⁵ is independently —C(O)O—. In embodiments, L⁵ is independently —OC(O)—. In embodiments, L⁵ is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁵ is independently substituted or unsubstituted alkylene. In embodiments, L⁵ is independently unsubstituted alkylene. In embodiments, L⁵ is independently unsubstituted methylene. In embodiments, L⁵ is independently unsubstituted ethylene. In embodiments, L⁵ is independently unsubstituted propylene. In embodiments, L⁵ is independently substituted or unsubstituted heteroalkylene. In embodiments, L⁵ is independently unsubstituted heteroalkylene. In embodiments, L⁵ is independently substituted or unsubstituted cycloalkylene. In embodiments, L⁵ is independently unsubstituted cycloalkylene. In embodiments, L⁵ is independently substituted or unsubstituted heterocycloalkylene. In embodiments, L⁵ is independently unsubstituted heterocycloalkylene. In embodiments, L⁵ is independently substituted or unsubstituted arylene. In embodiments, L⁵ is independently unsubstituted phenylene. In embodiments, L⁵ is independently substituted or unsubstituted heteroarylene. In embodiments, L⁵ is independently unsubstituted heteroarylene. In embodiments, L⁵ is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L⁵ is independently substituted or unsubstituted C₁-C₆ alkylene. In embodiments, L⁵ is independently unsubstituted C₁-C₆ alkylene. In embodiments, L⁵ is independently unsubstituted methylene. In embodiments, L⁵ is independently unsubstituted ethylene. In embodiments, L⁵ is independently unsubstituted propylene. In embodiments, L⁵ is independently substituted or unsubstituted 2 to 6 membered heteroalkylene. In embodiments, L⁵ is independently unsubstituted 2 to 6 membered heteroalkylene. In embodiments, L⁵ is independently substituted or unsubstituted C₃-C₆ cycloalkylene. In embodiments, L⁵ is independently unsubstituted C₃-C₆ cycloalkylene. In embodiments, L⁵ is independently substituted or unsubstituted 3 to 6 membered heterocycloalkylene. In embodiments, L⁵ is independently unsubstituted 3 to 6 membered heterocycloalkylene. In embodiments, L⁵ is independently substituted or unsubstituted C₆-C₁₀ arylene. In embodiments, L⁵ is independently unsubstituted C₆-C₁₀ arylene. In embodiments, L⁵ is independently substituted phenylene. In embodiments, L⁵ is independently unsubstituted phenylene. In embodiments, L⁵ is independently substituted or unsubstituted 5 to 10 membered heteroarylene. In embodiments, L⁵ is independently substituted or unsubstituted 5 to 6 membered heteroarylene. In embodiments, L⁵ is independently unsubstituted 5 to 10 membered heteroarylene. In embodiments, L⁵ is independently unsubstituted 5 to 6 membered heteroarylene.

In embodiments, a substituted L⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁵ is substituted with a plurality of groups selected from substituent groups, size-limited substituent groups, and lower substituent groups; each substituent group, size-limited substituent group, and/or lower substituent group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, R¹ is a 14-3-3β K122 binding moiety (14-3-3beta). In embodiments, R¹ is a 14-3-3ε K123 binding moiety (14-3-3epsilon). In embodiments, R¹ is a 14-3-3η K125 binding moiety (14-3-3eta). In embodiments, R¹ is a 14-3-3γ K125 binding moiety (14-3-3gamma). In embodiments, R¹ is a 14-3-3σ K122 binding moiety (14-3-3sigma). In embodiments, R¹ is a 14-3-3τ K120 binding moiety (14-3-3tau). In embodiments, R¹ is a 14-3-3ζ K120 binding moiety (14-3-3zeta).

In embodiments, R¹ is a 14-3-3 K120 covalent binding moiety. In embodiments, R¹ is a 14-3-3 K120 non-covalent binding moiety.

In embodiments, R¹ is a 14-3-3 K120 covalent binding moiety.

In embodiments, R¹ is a 14-3-3β K122 covalent binding moiety (14-3-3beta). In embodiments, R¹ is a 14-3-3ε K123 covalent binding moiety (14-3-3epsilon). In embodiments, R¹ is a 14-3-3η K125 covalent binding moiety (14-3-3eta). In embodiments, R¹ is a 14-3-3γ K125 covalent binding moiety (14-3-3gamma). In embodiments, R¹ is a 14-3-3σ K122 covalent binding moiety (14-3-3sigma). In embodiments, R¹ is a 14-3-3τ K120 covalent binding moiety (14-3-3tau). In embodiments, R¹ is a 14-3-3ζ K120 covalent binding moiety (14-3-3zeta).

In embodiments, R¹ is a 14-3-3 K120 non-covalent binding moiety.

In embodiments, R¹ is a 14-3-3β K122 non-covalent binding moiety (14-3-3beta). In embodiments, R¹ is a 14-3-3ε K123 non-covalent binding moiety (14-3-3epsilon). In embodiments, R¹ is a 14-3-3η K125 non-covalent binding moiety (14-3-3eta). In embodiments, R¹ is a 14-3-3γ K125 non-covalent binding moiety (14-3-3gamma). In embodiments, R¹ is a 14-3-3σ K122 non-covalent binding moiety (14-3-3sigma). In embodiments, R¹ is a 14-3-3τ K120 non-covalent binding moiety (14-3-3tau). In embodiments, R¹ is a 14-3-3ζ K120 non-covalent binding moiety (14-3-3zeta).

In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂RID, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C4-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N3, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 12 membered, 5 to 10 membered, 5 to 9 membered, or 5 to 6 membered).

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{1A} and R^{1B} substituents bonded to the same nitrogen atom are joined is substituted, it is substituted with at least one substituent group. In embodiments, when the ring formed when R^{1A} and R^{1B} substituents bonded to the same nitrogen atom are joined is substituted, it is substituted with at least one size-limited substituent group. In embodiments, when the ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1A} is independently hydrogen. In embodiments, R^{1B} is independently hydrogen. In embodiments, R^{1C} is independently hydrogen. In embodiments, R^{1D} is independently hydrogen.

In embodiments, R^{1A} is independently unsubstituted C₁-C₄ alkyl. In embodiments, R^{1B} is independently unsubstituted C₁-C₄ alkyl. In embodiments, R^{1C} is independently unsubstituted C₁-C₄ alkyl. In embodiments, R^{1D} is independently unsubstituted C₁-C₄ alkyl.

X¹ is independently —F, —Cl, —Br, or —I.

In embodiments, X¹ is independently —F. In embodiments, X¹ is independently —Cl. In embodiments, X¹ is independently —Br. In embodiments, X¹ is independently —I.

n1 is independently an integer from 0 to 4.

In embodiments, n1 is independently 0. In embodiments, n1 is independently 1. In embodiments, n1 is independently 2. In embodiments, n1 is independently 3. In embodiments, n1 is independently 4.

m1 and v1 are independently 1 or 2.

In embodiments, m1 is independently 1. In embodiments, m1 is independently 2. In embodiments, v1 is independently 1. In embodiments, v1 is independently 2.

In embodiments, R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R¹ is

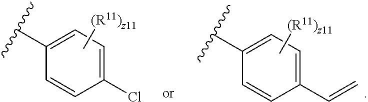

R¹¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered); two adjacent R¹¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

z11 is an integer from 0 to 4. In embodiments, z11 is 0. In embodiments, z11 is 1. In embodiments, z11 is 2. In embodiments, z11 is 3. In embodiments, z11 is 4.

In embodiments, R¹ is

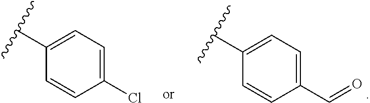 [SMILES: *c1ccc(Cl)cc1 ; *c1ccc(C=O)cc1]

In embodiments, R¹ is

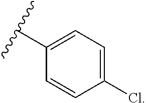 [SMILES: *c1ccc(Cl)cc1]

In embodiments, R¹ is

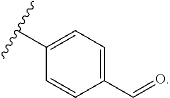 [SMILES: *c1ccc(C=O)cc1]

In embodiments, R¹ is

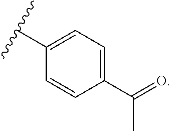 [SMILES: *c1ccc(C(C)=O)cc1]

In embodiments, R¹ is

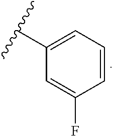 [SMILES: *c1cccc(F)c1]

In embodiments, R¹ is

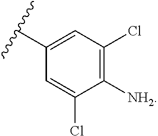 [SMILES: *c1cc(Cl)c(N)c(Cl)c1]

In embodiments, R¹ is —C(O)H. In embodiments, R¹ is —C(O)CH₃. In embodiments, R¹ is —Cl. In embodiments, R¹ is —F. In embodiments, R¹ is halogen.

In embodiments, R¹ is -L^{1A}-L^{1B}E.

L^{1A} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L^{1A} is independently a bond.

In embodiments, L^{1A} is independently —S(O)₂—. In embodiments, L^{1A} is independently —NH—. In embodiments, L^{1A} is independently —O—. In embodiments, L^{1A} is independently —S—. In embodiments, L^{1A} is independently —C(O)—. In embodiments, L^{1A} is independently —NHS(O)₂—. In embodiments, L^{1A} is independently —S(O)₂NH—. In embodiments, L^{1A} is independently —C(O)NH—. In embodiments, L^{1A} is independently —NHC(O)—. In embodiments, L^{1A} is independently —NHC(O)NH—. In embodiments, L^{1A} is independently —C(O)O—. In embodiments, L^{1A} is independently —OC(O)—.

In embodiments, L^{1A} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{1A} is independently substituted or unsubstituted alkylene. In embodiments, L^{1A} is independently unsubstituted alkylene. In embodiments, L^{1A} is independently unsubstituted methylene. In embodiments, L^{1A} is independently unsubstituted ethylene. In embodiments, L^{1A} is independently unsubstituted propylene. In embodiments, L^{1A} is independently substituted or unsubstituted heteroalkylene. In embodiments, L^{1A} is independently unsubstituted heteroalkylene. In embodiments, L^{1A} is independently substituted or unsubstituted cycloalkylene. In embodiments, L^{1A} is independently unsubstituted cycloalkylene. In embodiments, L^{1A} is independently substituted or unsubstituted heterocycloalkylene. In embodiments, L^{1A} is independently unsubstituted heterocycloalkylene. In embodiments, L^{1A} is independently substituted or unsubstituted arylene. In embodiments, L^{1A} is independently unsubstituted phenylene. In embodiments, L^{1A} is independently substituted or unsubstituted heteroarylene. In embodiments, L^{1A} is independently unsubstituted heteroarylene. In embodiments, L^{1A} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{1A} is independently substituted or unsubstituted C₁-C₆ alkylene. In embodiments, L^{1A} is independently unsubstituted C₁-C₆ alkylene. In embodiments, L^{1A} is independently unsubstituted methylene. In embodiments, L^{1A} is independently unsubstituted ethylene. In embodiments, L^{1A} is independently unsubstituted propylene. In embodiments, L^{1A} is independently substituted or unsubstituted 2 to 6 membered heteroalkylene. In embodiments, L^{1A} is independently unsubstituted 2 to 6 membered heteroalkylene. In embodiments, L^{1A} is independently substituted or unsubstituted C₃-C₆ cycloalkylene. In embodiments, L^{1A} is independently unsubstituted C₃-C₆ cycloalkylene. In embodiments, L^{1A} is independently substituted or unsubstituted 3 to 6 membered heterocycloalkylene. In embodiments, L^{1A} is independently unsubstituted 3 to 6 membered heterocycloalkylene. In embodiments, L^{1A} is independently substituted or unsubstituted C₆-C₁₀ arylene. In embodiments, L^{1A} is independently unsubstituted C₆-C₁₀ arylene. In embodiments, L^{1A} is independently substituted phenylene. In embodiments, L^{1A} is independently unsubstituted phenylene. In embodiments, L^{1A} is independently substituted or unsubstituted 5 to 10 membered heteroarylene. In embodiments, L^{1A} is independently substituted or unsubstituted 5 to 6 membered heteroarylene. In embodiments, L^{1A} is independently unsubstituted 5 to 10 membered heteroarylene. In embodiments, L^{1A} is independently unsubstituted 5 to 6 membered heteroarylene.

In embodiments, a substituted L^{1A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{1A} is substituted with a plurality of groups selected from substituent groups, size-limited substituent groups, and lower substituent groups; each substituent group, size-limited substituent group, and/or lower substituent group may optionally be different. In embodiments, when L^{1A} is substituted, it is substituted with at least one substituent group. In embodiments, when L^{1A} is substituted, it is substituted with at least one size-limited substituent group. In embodiments, when L^{1A} is substituted, it is substituted with at least one lower substituent group.

LB is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L^{1B} is independently a bond, —NH—, —C(O)NH—, —NHC(O)—, —NHC(O)NH—, substituted or unsubstituted heteroalkylene (e.g., 2 to 8 membered, 2 to 6 membered, 4 to 6 membered, 2 to 3 membered, or 4 to 5 membered), substituted or unsubstituted heterocycloalkylene (e.g., 3 to 8 membered, 3 to 6 membered, 4 to 6 membered, 4 to 5 membered, or 5 to 6 membered), or substituted or unsubstituted heteroarylene (e.g., 5 to 12 membered, 5 to 10 membered, 5 to 9 membered, or 5 to 6 membered).

In embodiments, L^{1B} is independently a bond.

In embodiments, L^{1B} is independently —NH—. In embodiments, L^{1B} is independently —C(O)NH—. In embodiments, L^{1B} is independently —NHC(O)—. In embodiments, L^{1B} is independently —NHC(O)NH—.

In embodiments, L^{1B} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{1B} is independently substituted or unsubstituted alkylene. In embodiments, L^{1B} is independently unsubstituted alkylene. In embodiments, L^{1B} is independently unsubstituted methylene. In embodiments, L^{1B} is independently unsubstituted ethylene. In embodiments, L^{1B} is independently unsubstituted propylene. In embodiments, L^{1B} is independently substituted or unsubstituted heteroalkylene. In embodiments, L^{1B} is independently unsubstituted heteroalkylene. In embodiments, L^{1B} is independently substituted or unsubstituted cycloalkylene. In embodiments, L^{1B} is independently unsubstituted cycloalkylene. In embodiments, L^{1B} is independently substituted or unsubstituted heterocycloalkylene. In embodiments, L^{1B} is independently unsubstituted heterocycloalkylene. In embodiments, L^{1B} is independently substituted or unsubstituted arylene. In embodiments, L^{1B} is independently unsubstituted phenylene. In embodiments, L^{1B} is independently substituted or unsubstituted heteroarylene. In embodiments, L^{1B} is independently unsubstituted heteroarylene. In embodiments, L^{1B} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{1B} is independently substituted or unsubstituted C₁-C₆ alkylene. In embodiments, L^{1B} is independently unsubstituted C₁-C₆ alkylene. In embodiments, L^{1B} is independently unsubstituted methylene. In embodiments, L^{1B} is independently unsubstituted ethylene. In embodiments, L^{1B} is independently unsubstituted propylene. In embodiments, L^{1B} is independently substituted or unsubstituted 2 to 6 membered heteroalkylene. In embodiments, L^{1B} is independently unsubstituted 2 to 6 membered heteroalkylene. In embodiments, L^{1B} is independently substituted or unsubstituted C₃-C₆ cycloalkylene. In embodiments, L^{1B} is independently unsubstituted C₃-C₆ cycloalkylene. In embodiments, L^{1B} is independently substituted or unsubstituted 3 to 6 membered heterocycloalkylene. In embodiments, L^{1B} is independently unsubstituted 3 to 6 membered heterocycloalkylene. In embodiments, L^{1B} is independently substituted or unsubstituted C₆-C₁₀ arylene. In embodiments, L^{1B} is independently unsubstituted C₆-C₁₀ arylene. In embodiments, L^{1B} is independently substituted phenylene. In embodiments, L^{1B} is independently unsubstituted phenylene. In embodiments, L^{1B} is independently substituted or unsubstituted 5 to 10 membered heteroarylene. In embodiments, L^{1B} is independently substituted or unsubstituted 5 to 6 membered heteroarylene. In embodiments, L^{1B} is independently unsubstituted 5 to 10 membered heteroarylene. In embodiments, L^{1B} is independently unsubstituted 5 to 6 membered heteroarylene.

In embodiments, a substituted L^{1B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{1B} is substituted with a plurality of groups selected from substituent groups, size-limited substituent groups, and lower substituent groups; each substituent group, size-limited substituent group, and/or lower substituent group may optionally be different. In embodiments, when L^{1B} is substituted, it is substituted with at least one substituent group. In embodiments, when L^{1B} is substituted, it is substituted with at least one size-limited substituent group. In embodiments, when L^{1B} is substituted, it is substituted with at least one lower substituent group.

E is a covalent lysine modifier moiety. In embodiments, E is a 14-3-3 K120 covalent binding moiety. In embodiments, E is

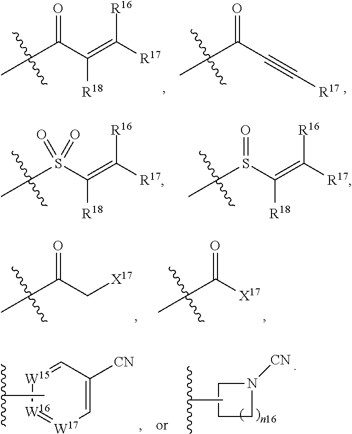

R¹⁶, R¹⁷, and R¹⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

W¹⁵, W¹⁶, and W¹⁷ are independently CH or N. In embodiments, W¹⁵ is independently CH. In embodiments, W¹⁵ is independently N. In embodiments, W¹⁶ is independently CH. In embodiments, W¹⁶ is independently N. In embodiments, W¹⁷ is independently CH. In embodiments, W¹⁷ is independently N. n16 is an integer from 1 to 5. In embodiments, n16 is 1. In embodiments, n16 is 2. In embodiments, n16 is 3. In embodiments, n16 is 4. In embodiments, n16 is 5.

In embodiments, E is a monovalent form of a substituted or unsubstituted aryl sulfonyl halide, substituted or unsubstituted aryl sulfonyl fluoride, substituted or unsubstituted aryl fluorosulfate, substituted or unsubstituted dihalide triazine, substituted or unsubstituted activated ester, substituted or unsubstituted activated thioester, substituted or unsubstituted activated amide, substituted or unsubstituted activated phosphor-amide, substituted or unsubstituted aromatic aldehyde, substituted or unsubstituted aromatic ketone, substituted or unsubstituted isocyanates, substituted or unsubstituted isothiocyanates, or substituted or unsubstituted benzoyl fluoride. In embodiments, E is a monovalent form of a substituted or unsubstituted aryl sulfonyl halide. In embodiments, E is a monovalent form of a substituted or unsubstituted aryl sulfonyl fluoride. In embodiments, E is a monovalent form of a substituted or unsubstituted aryl fluorosulfate. In embodiments, E is a monovalent form of a substituted or unsubstituted dihalide triazine. In embodiments, E is a monovalent form of a substituted or unsubstituted activated ester. In embodiments, E is a monovalent form of a substituted or unsubstituted activated thioester. In embodiments, E is a monovalent form of a substituted or unsubstituted activated amide. In embodiments, E is a monovalent form of a substituted or unsubstituted activated phosphor-amide. In embodiments, E is a monovalent form of a substituted or unsubstituted aromatic aldehyde. In embodiments, E is a monovalent form of a substituted or unsubstituted aromatic ketone. In embodiments, E is a monovalent form of a substituted or unsubstituted isocyanates. In embodiments, E is a monovalent form of a substituted or unsubstituted isothiocyanates. In embodiments, E is a monovalent form of a substituted or unsubstituted benzoyl fluoride. In embodiments, E is a monovalent form of an unsubstituted aryl sulfonyl halide, unsubstituted aryl sulfonyl fluoride, unsubstituted aryl fluorosulfate, unsubstituted dihalide triazine, unsubstituted activated ester, unsubstituted activated thioester, unsubstituted activated amide, unsubstituted activated phosphor-amide, unsubstituted aromatic aldehyde, unsubstituted aromatic ketone, unsubstituted isocyanates, unsubstituted isothiocyanates, or unsubstituted benzoyl fluoride.

In embodiments, a substituted E (e.g., substituted aryl sulfonyl halide, substituted aryl sulfonyl fluoride, substituted aryl fluorosulfate, substituted dihalide triazine, substituted activated ester, substituted activated thioester, substituted activated amide, substituted activated phosphor-amide, substituted aromatic aldehyde, substituted aromatic ketone, substituted isocyanates, substituted isothiocyanates, and/or substituted benzoyl fluoride) is substituted with at least one substituent group, size-limited substituent group, or lower substituent group; wherein if the substituted E is substituted with a plurality of groups selected from substituent groups, size-limited substituent groups, and lower substituent groups; each substituent group, size-limited substituent group, and/or lower substituent group may optionally be different. In embodiments, when E is substituted, it is substituted with at least one substituent group. In embodiments, when E is substituted, it is substituted with at least one size-limited substituent group. In embodiments, when E is substituted, it is substituted with at least one lower substituent group.

In embodiments, R¹⁶ is independently hydrogen, substituted or unsubstituted C₁-C₄ alkyl, or substituted or unsubstituted 2 to 4 membered heteroalkyl. In embodiments, R¹⁶ is independently hydrogen. In embodiments, R¹⁶ is independently substituted or unsubstituted C₁-C₄ alkyl. In embodiments, R¹⁶ is independently substituted or unsubstituted 2 to 4 membered heteroalkyl. In embodiments, R¹⁶ is independently unsubstituted C₁-C₄ alkyl. In embodiments, R¹⁶ is independently unsubstituted 2 to 4 membered heteroalkyl. In embodiments, R¹⁶ is independently substituted or unsubstituted C₁-C₆ alkyl or substituted or unsubstituted 2 to 6 membered heteroalkyl. In embodiments, R¹⁶ is independently —Cl. In embodiments, R¹⁶ is independently —Br. In embodiments, R¹⁶ is independently —F. In embodiments, R¹⁶ is independently —I. In embodiments, R¹⁶ is independently —CH₃. In embodiments, R¹⁶ is independently —CCl₃. In embodiments, R¹⁶ is independently —CBr₃. In embodiments, R¹⁶ is independently —CF₃. In embodiments, R¹⁶ is independently —CI₃. In embodiments, R¹⁶ is independently —CHCl₂. In embodiments, R¹⁶ is independently —CHBr₂. In embodiments, R¹⁶ is independently —CHF₂. In embodiments, R¹⁶ is independently —CHI₂. In embodiments, R¹⁶ is independently —CH₂C1. In embodiments, R¹⁶ is independently —CH₂Br. In embodiments, R¹⁶ is independently —CH₂F. In embodiments, R¹⁶ is independently —CH₂I. In embodiments, R¹⁶ is independently —CN. In embodiments, R¹⁶ is independently —OCH₃. In embodiments, R¹⁶ is independently —NH₂. In embodiments, R¹⁶ is independently —COOH. In embodiments, R¹⁶ is independently —COCH₃. In embodiments, R¹⁶ is independently —CONH₂. In embodiments, R¹⁶ is independently —OCCl₃. In embodiments, R¹⁶ is independently —OCF₃. In embodiments, R¹⁶ is independently —OCBr₃. In embodiments, R¹⁶ is independently —OCI₃. In embodiments, R¹⁶ is independently —OCHCl₂. In embodiments, R¹⁶ is independently —OCHBr₂. In embodiments, R¹⁶ is independently —OCHI₂. In embodiments, R¹⁶ is independently —OCHF₂. In embodiments, R¹⁶ is independently —OCH₂Cl. In embodiments, R¹⁶ is independently —OCH₂Br. In embodiments, R¹⁶ is independently —OCH₂I. In embodiments, R¹⁶ is independently —OCH₂F. In embodiments, R¹⁶ is independently unsubstituted methyl. In embodiments, R¹⁶ is independently —OCH₃. In embodiments, R¹⁶ is independently —OCH₂CH₃. In embodiments, R¹⁶ is independently —OCH(CH₃)₂. In embodiments, R¹⁶ is independently —OC(CH₃)₃. In embodiments, R¹⁶ is independently —CH₃. In embodiments, R¹⁶ is independently —CH₂CH₃. In embodiments, R¹⁶ is independently —CH(CH₃)₂. In embodiments, R¹⁶ is independently —C(CH₃)₃. In embodiments, R¹⁶ is independently —C(O)CH₃. In embodiments, R¹⁶ is independently —C(O)CH₂CH₃. In embodiments, R¹⁶ is independently —C(O)CH(CH₃)₂. In embodiments, R¹⁶ is independently —C(O)C(CH₃)₃.

In embodiments, R¹⁶ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁶ is independently substituted or unsubstituted alkyl. In embodiments, R¹⁶ is independently unsubstituted alkyl. In embodiments, R¹⁶ is independently unsubstituted methyl. In embodiments, R¹⁶ is independently unsubstituted ethyl. In embodiments, R¹⁶ is independently unsubstituted propyl. In embodiments, R¹⁶ is independently substituted or unsubstituted heteroalkyl. In embodiments, R¹⁶ is independently unsubstituted heteroalkyl. In embodiments, R¹⁶ is independently substituted or unsubstituted cycloalkyl. In embodiments, R¹⁶ is independently unsubstituted cycloalkyl. In embodiments, R¹⁶ is independently substituted or unsubstituted heterocycloalkyl. In embodiments, R¹⁶ is independently unsubstituted heterocycloalkyl. In embodiments, R¹⁶ is independently substituted or unsubstituted aryl. In embodiments, R¹⁶ is independently unsubstituted phenyl. In embodiments, R¹⁶ is independently substituted or unsubstituted heteroaryl. In embodiments, R¹⁶ is independently unsubstituted heteroaryl. In embodiments, R¹⁶ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁶ is independently substituted or unsubstituted C₁-C₆ alkyl. In embodiments, R¹⁶ is independently unsubstituted C₁-C₆ alkyl. In embodiments, R¹⁶ is independently unsubstituted methyl. In embodiments, R¹⁶ is independently unsubstituted ethyl. In embodiments, R¹⁶ is independently unsubstituted propyl. In embodiments, R¹⁶ is independently substituted or unsubstituted 2 to 6 membered heteroalkyl. In embodiments, R¹⁶ is independently unsubstituted 2 to 6 membered heteroalkyl. In embodiments, R¹⁶ is independently substituted or unsubstituted C₃-C₆ cycloalkyl. In embodiments, R¹⁶ is independently unsubstituted C₃-C₆ cycloalkyl. In embodiments, R¹⁶ is independently substituted or unsubstituted 3 to 6 membered heterocycloalkyl. In embodiments, R¹⁶ is independently unsubstituted 3 to 6 membered heterocycloalkyl. In embodiments, R¹⁶ is independently substituted or unsubstituted C₆-C₁₀ aryl. In embodiments, R¹⁶ is independently unsubstituted C₆-C₁₀ aryl. In embodiments, R¹⁶ is independently substituted phenyl. In embodiments, R¹⁶ is independently unsubstituted phenyl. In embodiments, R¹⁶ is independently substituted or unsubstituted 5 to 10 membered heteroaryl. In embodiments, R¹⁶ is independently substituted or unsubstituted 5 to 6 membered heteroaryl. In embodiments, R¹⁶ is independently unsubstituted 5 to 10 membered heteroaryl. In embodiments, R¹⁶ is independently unsubstituted 5 to 6 membered heteroaryl.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, R¹⁷ is independently hydrogen, substituted or unsubstituted C₁-C₄ alkyl, or substituted or unsubstituted 2 to 4 membered heteroalkyl. In embodiments, R¹⁷ is independently hydrogen. In embodiments, R¹⁷ is independently substituted or unsubstituted C₁-C₄ alkyl. In embodiments, R¹⁷ is independently substituted or unsubstituted 2 to 4 membered heteroalkyl. In embodiments, R¹⁷ is independently unsubstituted C₁-C₄ alkyl. In embodiments, R¹⁷ is independently unsubstituted 2 to 4 membered heteroalkyl. In embodiments, R¹⁷ is independently substituted or unsubstituted C₁-C₆ alkyl or substituted or unsubstituted 2 to 6 membered heteroalkyl. In embodiments, R¹⁷ is independently —Cl. In embodiments, R¹⁷ is independently —Br. In embodiments, R¹⁷ is independently —F. In embodiments, R¹⁷ is independently —I. In embodiments, R¹⁷ is independently —CH₃. In embodiments, R¹⁷ is independently —CCl₃. In embodiments, R¹⁷ is independently —CBr₃. In embodiments, R¹⁷ is independently —CF₃. In embodiments, R¹⁷ is independently —Cl₃. In embodiments, R¹⁷ is independently —CHCl₂. In embodiments, R¹⁷ is independently —CHBr₂. In embodiments, R¹⁷ is independently —CHF₂. In embodiments, R¹⁷ is independently —CHI₂. In embodiments, R¹⁷ is independently —CH₂C1. In embodiments, R¹⁷ is independently —CH₂Br. In embodiments, R¹⁷ is independently —CH₂F. In embodiments, R¹⁷ is independently —CH₂I. In embodiments, R¹⁷ is independently —CN. In embodiments, R¹⁷ is independently —OCH₃. In embodiments, R¹⁷ is independently —NH₂. In embodiments, R¹⁷ is independently —COOH. In embodiments, R¹⁷ is independently —COCH₃. In embodiments, R¹⁷ is independently —CONH₂. In embodiments, R¹⁷ is independently —OCCl₃. In embodiments, R¹⁷ is independently —OCF₃. In embodiments, R¹⁷ is independently —OCBr₃. In embodiments, R¹⁷ is independently —OCI₃. In embodiments, R¹⁷ is independently —OCHCl₂. In embodiments, R¹⁷ is independently —OCHBr₂. In embodiments, R¹⁷ is independently —OCHI₂. In embodiments, R¹⁷ is independently —OCHF₂. In embodiments, R¹⁷ is independently —OCH₂Cl. In embodiments, R¹⁷ is independently —OCH₂Br. In embodiments, R¹⁷ is independently —OCH₂I. In embodiments, R¹⁷ is independently —OCH₂F. In embodiments, R¹⁷ is independently unsubstituted methyl. In embodiments, R¹⁷ is independently —OCH₃. In embodiments, R¹⁷ is independently —OCH₂CH₃. In embodiments, R¹⁷ is independently —OCH(CH₃)₂. In embodiments, R¹⁷ is independently —OC(CH₃)₃. In embodiments, R¹⁷ is independently —CH₃. In embodiments, R¹⁷ is independently —CH₂CH₃. In embodiments, R¹⁷ is independently —CH(CH₃)₂. In embodiments, R¹⁷ is independently —C(CH₃)₃. In embodiments, R¹⁷ is independently —C(O)CH₃. In embodiments, R¹⁷ is independently —C(O)CH₂CH₃. In embodiments, R¹⁷ is independently —C(O)CH(CH₃)₂. In embodiments, R¹⁷ is independently —C(O)C(CH₃)₃.

In embodiments, R¹⁷ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁷ is independently substituted or unsubstituted alkyl. In embodiments, R¹⁷ is independently unsubstituted alkyl. In embodiments, R¹⁷ is independently unsubstituted methyl. In embodiments, R¹⁷ is independently unsubstituted ethyl. In embodiments, R¹⁷ is independently unsubstituted propyl. In embodiments, R¹⁷ is independently substituted or unsubstituted heteroalkyl. In embodiments, R¹⁷ is independently unsubstituted heteroalkyl. In embodiments, R¹⁷ is independently substituted or unsubstituted cycloalkyl. In embodiments, R¹⁷ is independently unsubstituted cycloalkyl. In embodiments, R¹⁷ is independently substituted or unsubstituted heterocycloalkyl. In embodiments, R¹⁷ is independently unsubstituted heterocycloalkyl. In embodiments, R¹⁷ is independently substituted or unsubstituted aryl. In embodiments, R¹⁷ is independently unsubstituted phenyl. In embodiments, R¹⁷ is independently substituted or unsubstituted heteroaryl. In embodiments, R¹⁷ is independently unsubstituted heteroaryl. In embodiments, R¹⁷ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁷ is independently substituted or unsubstituted C₁-C₆ alkyl. In embodiments, R¹⁷ is independently unsubstituted C₁-C₆ alkyl. In embodiments, R¹⁷ is independently unsubstituted methyl. In embodiments, R¹⁷ is independently unsubstituted ethyl. In embodiments, R¹⁷ is independently unsubstituted propyl. In embodiments, R¹⁷ is independently substituted or unsubstituted 2 to 6 membered heteroalkyl. In embodiments, R¹⁷ is independently unsubstituted 2 to 6 membered heteroalkyl. In embodiments, R¹⁷ is independently substituted or unsubstituted C₃-C₆ cycloalkyl. In embodiments, R¹⁷ is independently unsubstituted C₃-C₆ cycloalkyl. In embodiments, R¹⁷ is independently substituted or unsubstituted 3 to 6 membered heterocycloalkyl. In embodiments, R¹⁷ is independently unsubstituted 3 to 6 membered heterocycloalkyl. In embodiments, R¹⁷ is independently substituted or unsubstituted C₆-C₁₀ aryl. In embodiments, R¹⁷ is independently unsubstituted C₆-C₁₀ aryl. In embodiments, R¹⁷ is independently substituted phenyl. In embodiments, R¹⁷ is independently unsubstituted phenyl. In embodiments, R¹⁷ is independently substituted or unsubstituted 5 to 10 membered heteroaryl. In embodiments, R¹⁷ is independently substituted or unsubstituted 5 to 6 membered heteroaryl. In embodiments, R¹⁷ is independently unsubstituted 5 to 10 membered heteroaryl. In embodiments, R¹⁷ is independently unsubstituted 5 to 6 membered heteroaryl.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁸ is independently hydrogen, substituted or unsubstituted C₁-C₄ alkyl, or substituted or unsubstituted 2 to 4 membered heteroalkyl. In embodiments, R¹⁸ is independently hydrogen. In embodiments, R¹⁸ is independently substituted or unsubstituted C₁-C₄ alkyl. In embodiments, R¹⁸ is independently substituted or unsubstituted 2 to 4 membered heteroalkyl. In embodiments, R¹⁸ is independently unsubstituted C₁-C₄ alkyl. In embodiments, R¹⁸ is independently unsubstituted 2 to 4 membered heteroalkyl. In embodiments, R¹⁸ is independently substituted or unsubstituted C₁-C₆ alkyl or substituted or unsubstituted 2 to 6 membered heteroalkyl. In embodiments, R¹⁸ is independently —Cl. In embodiments, R¹⁸ is independently —Br. In embodiments, R¹⁸ is independently —F. In embodiments, R¹⁸ is independently —I. In embodiments, R¹⁸ is independently —CH₃. In embodiments, R¹⁸ is independently —CCl₃. In embodiments, R¹⁸ is independently —CBr₃. In embodiments, R¹⁸ is independently —CF₃. In embodiments, R¹⁸ is independently —CI₃. In embodiments, R¹⁸ is independently —CHCl₂. In embodiments, R¹⁸ is independently —CHBr₂. In embodiments, R¹⁸ is independently —CHF₂. In embodiments, R¹⁸ is independently —CHI₂. In embodiments, R¹⁸ is independently —CH₂C1. In embodiments, R¹⁸ is independently —CH₂Br. In embodiments, R¹⁸ is independently —CH₂F. In embodiments, R¹⁸ is independently —CH₂I. In embodiments, R¹⁸ is independently —CN. In embodiments, R¹⁸ is independently —OCH₃. In embodiments, R¹⁸ is independently —NH₂. In embodiments, R¹⁸ is independently —COOH. In embodiments, R¹⁸ is independently —COCH₃. In embodiments, R¹⁸ is independently —CONH₂. In embodiments, R¹⁸ is independently —OCCl₃. In embodiments, R¹⁸ is independently —OCF₃. In embodiments, R¹⁸ is independently —OCBr₃. In embodiments, R¹⁸ is independently —OCI₃. In embodiments, R¹⁸ is independently —OCHCl₂. In embodiments, R¹⁸ is independently —OCHBr₂. In embodiments, R¹⁸ is independently —OCHI₂. In embodiments, R¹⁸ is independently —OCHF₂. In embodiments, R¹⁸ is independently —OCH₂Cl. In embodiments, R¹⁸ is independently —OCH₂Br. In embodiments, R¹⁸ is independently —OCH₂I. In embodiments, R¹⁸ is independently —OCH₂F. In embodiments, R¹⁸ is independently unsubstituted methyl. In embodiments, R¹⁸ is independently —OCH₃. In embodiments, R¹⁸ is independently —OCH₂CH₃. In embodiments, R¹⁸ is independently —OCH(CH₃)₂. In embodiments, R¹⁸ is independently —OC(CH₃)₃. In embodiments, R¹⁸ is independently —CH₃. In embodiments, R¹⁸ is independently —CH₂CH₃. In embodiments, R¹⁸ is independently —CH(CH₃)₂. In embodiments, R¹⁸ is independently —C(CH₃)₃. In embodiments, R¹⁸ is independently —C(O)CH₃. In embodiments, R¹⁸ is independently —C(O)CH₂CH₃. In embodiments, R¹⁸ is independently —C(O)CH(CH₃)₂. In embodiments, R¹⁸ is independently —C(O)C(CH₃)₃.

In embodiments, R¹⁸ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁸ is independently substituted or unsubstituted alkyl. In embodiments, R¹⁸ is independently unsubstituted alkyl. In embodiments, R¹⁸ is independently unsubstituted methyl. In embodiments, R¹⁸ is independently unsubstituted ethyl. In embodiments, R¹⁸ is independently unsubstituted propyl. In embodiments, R¹⁸ is independently substituted or unsubstituted heteroalkyl. In embodiments, R¹⁸ is independently unsubstituted heteroalkyl. In embodiments, R¹⁸ is independently substituted or unsubstituted cycloalkyl. In embodiments, R¹⁸ is independently unsubstituted cycloalkyl. In embodiments, R¹⁸ is independently substituted or unsubstituted heterocycloalkyl. In embodiments, R¹⁸ is independently unsubstituted heterocycloalkyl. In embodiments, R¹⁸ is independently substituted or unsubstituted aryl. In embodiments, R¹⁸ is independently unsubstituted phenyl. In embodiments, R¹⁸ is independently substituted or unsubstituted heteroaryl. In embodiments, R¹⁸ is independently unsubstituted heteroaryl. In embodiments, R¹⁸ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁸ is independently substituted or unsubstituted C₁-C₆ alkyl. In embodiments, R¹⁸ is independently unsubstituted C₁-C₆ alkyl. In embodiments, R¹⁸ is independently unsubstituted methyl. In embodiments, R¹⁸ is independently unsubstituted ethyl. In embodiments, R¹⁸ is independently unsubstituted propyl. In embodiments, R¹⁸ is independently substituted or unsubstituted 2 to 6 membered heteroalkyl. In embodiments, R¹⁸ is independently unsubstituted 2 to 6 membered heteroalkyl. In embodiments, R¹⁸ is independently substituted or unsubstituted C₃-C₆ cycloalkyl. In embodiments, R¹⁸ is independently unsubstituted C₃-C₆ cycloalkyl. In embodiments, R¹⁸ is independently substituted or unsubstituted 3 to 6 membered heterocycloalkyl. In embodiments, R¹⁸ is independently unsubstituted 3 to 6 membered heterocycloalkyl. In embodiments, R¹⁸ is independently substituted or unsubstituted C₆-C₁₀ aryl. In embodiments, R¹⁸ is independently unsubstituted C₆-C₁₀ aryl. In embodiments, R¹⁸ is independently substituted phenyl. In embodiments, R¹⁸ is independently unsubstituted phenyl. In embodiments, R¹⁸ is independently substituted or unsubstituted 5 to 10 membered heteroaryl. In embodiments, R¹⁸ is independently substituted or unsubstituted 5 to 6 membered heteroaryl. In embodiments, R¹⁸ is independently unsubstituted 5 to 10 membered heteroaryl. In embodiments, R¹⁸ is independently unsubstituted 5 to 6 membered heteroaryl.

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

X¹⁷ is independently —F, —Cl, —Br, or —I.

In embodiments, X¹⁷ is independently —F. In embodiments, X¹⁷ is independently —Cl. In embodiments, X¹⁷ is independently —Br. In embodiments, X¹⁷ is independently —I.

In embodiments, E is

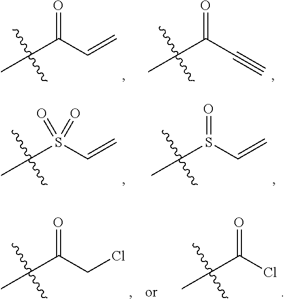

In embodiments, E is

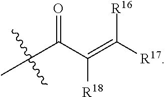

In embodiments, E is

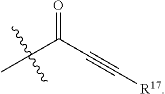

In embodiments, E is

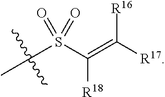

In embodiments, E is

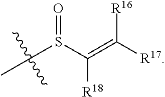

In embodiments, E is

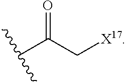

In embodiments, E is

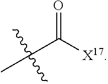

In embodiments, E is

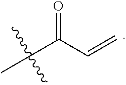 [SMILES: *C(=O)C=C]

In embodiments, E is

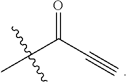 [SMILES: *C(=O)C#C]

In embodiments, E is

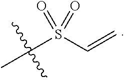 [SMILES: *S(=O)(=O)C=C]

In embodiments, E is

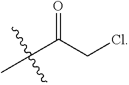 [SMILES: *C(=O)CCl]

In embodiments, E is

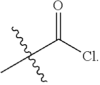 [SMILES: *C(=O)Cl]

In embodiments, E is

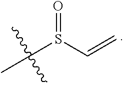 [SMILES: *S(=O)C=C]

In embodiments, E is

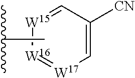

and W¹⁵, W¹⁶, and W⁷ are as described herein. In embodiments, E is

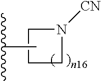

and n16 is as described herein.

In embodiments, E is

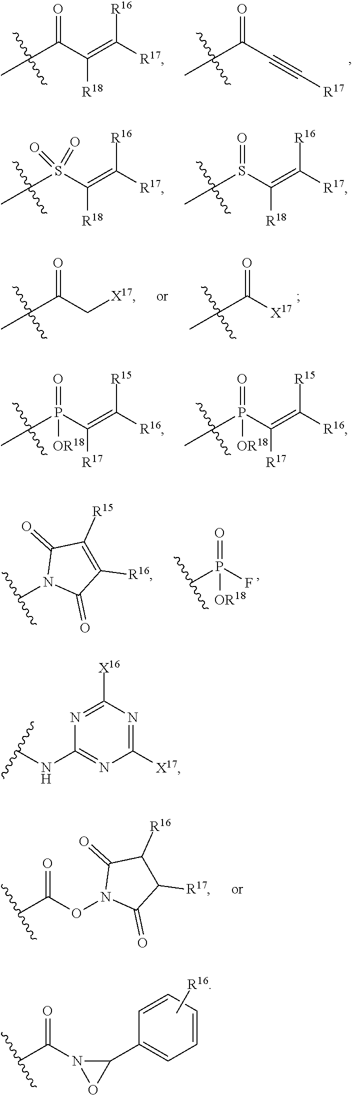

R¹⁶, R¹⁷, R¹⁸, and X¹⁷ are as described herein. X¹⁶ is independently a halogen. In embodiments, X¹⁶ is independently —Cl. In embodiments, X¹⁶ is independently —Br. In embodiments, X¹⁶ is independently —F. In embodiments, X¹⁶ is independently —I.

R¹⁵ is independently hydrogen, halogen, —CC₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁵ is independently hydrogen, substituted or unsubstituted C₁-C₄ alkyl, or substituted or unsubstituted 2 to 4 membered heteroalkyl. In embodiments, R¹⁵ is independently hydrogen. In embodiments, R¹⁵ is independently substituted or unsubstituted C₁-C₄ alkyl. In embodiments, R¹⁵ is independently substituted or unsubstituted 2 to 4 membered heteroalkyl. In embodiments, R¹⁵ is independently unsubstituted C₁-C₄ alkyl. In embodiments, R¹⁵ is independently unsubstituted 2 to 4 membered heteroalkyl. In embodiments, R¹⁵ is independently substituted or unsubstituted C₁-C₆ alkyl or substituted or unsubstituted 2 to 6 membered heteroalkyl. In embodiments, R¹⁵ is independently —Cl. In embodiments, R¹⁵ is independently —Br. In embodiments, R¹⁵ is independently —F. In embodiments, R¹⁵ is independently —I. In embodiments, R¹⁵ is independently —CH₃. In embodiments, R¹⁵ is independently —CCl₃. In embodiments, R¹⁵ is independently —CBr₃. In embodiments, R¹⁵ is independently —CF₃. In embodiments, R¹⁵ is independently —Cl₃. In embodiments, R¹⁵ is independently —CHCl₂. In embodiments, R¹⁵ is independently —CHBr₂. In embodiments, R¹⁵ is independently —CHF₂. In embodiments, R¹⁵ is independently —CHI₂. In embodiments, R¹⁵ is independently —CH₂C1. In embodiments, R¹⁵ is independently —CH₂Br. In embodiments, R¹⁵ is independently —CH₂F. In embodiments, R¹⁵ is independently —CH₂I. In embodiments, R¹⁵ is independently —CN. In embodiments, R¹⁵ is independently —OCH₃. In embodiments, R¹⁵ is independently —NH₂. In embodiments, R¹⁵ is independently —COOH. In embodiments, R¹⁵ is independently —COCH₃. In embodiments, R¹⁵ is independently —CONH₂. In embodiments, R¹⁵ is independently —OCCl₃. In embodiments, R¹⁵ is independently —OCF₃. In embodiments, R¹⁵ is independently —OCBr₃. In embodiments, R¹⁵ is independently —OCI₃. In embodiments, R¹⁵ is independently —OCHCl₂. In embodiments, R¹⁵ is independently —OCHBr₂. In embodiments, R¹⁵ is independently —OCHI₂. In embodiments, R¹⁵ is independently —OCHF₂. In embodiments, R¹⁵ is independently —OCH₂Cl. In embodiments, R¹⁵ is independently —OCH₂Br. In embodiments, R¹⁵ is independently —OCH₂I. In embodiments, R¹⁵ is independently —OCH₂F. In embodiments, R¹⁵ is independently unsubstituted methyl. In embodiments, R¹⁵ is independently —OCH₃. In embodiments, R¹⁵ is independently —OCH₂CH₃. In embodiments, R¹⁵ is independently —OCH(CH₃)₂. In embodiments, R¹⁵ is independently —OC(CH₃)₃. In embodiments, R¹⁵ is independently —CH₃. In embodiments, R¹⁵ is independently —CH₂CH₃. In embodiments, R¹⁵ is independently —CH(CH₃)₂. In embodiments, R¹⁵ is independently —C(CH₃)₃. In embodiments, R¹⁵ is independently —C(O)CH₃. In embodiments, R¹⁵ is independently —C(O)CH₂CH₃. In embodiments, R¹⁵ is independently —C(O)CH(CH₃)₂. In embodiments, R¹⁵ is independently —C(O)C(CH₃)₃.

In embodiments, R¹⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁵ is independently substituted or unsubstituted alkyl. In embodiments, R¹⁵ is independently unsubstituted alkyl. In embodiments, R¹⁵ is independently unsubstituted methyl. In embodiments, R¹⁵ is independently unsubstituted ethyl. In embodiments, R¹⁵ is independently unsubstituted propyl. In embodiments, R¹⁵ is independently substituted or unsubstituted heteroalkyl. In embodiments, R¹⁵ is independently unsubstituted heteroalkyl. In embodiments, R¹⁵ is independently substituted or unsubstituted cycloalkyl. In embodiments, R¹⁵ is independently unsubstituted cycloalkyl. In embodiments, R¹⁵ is independently substituted or unsubstituted heterocycloalkyl. In embodiments, R¹⁵ is independently unsubstituted heterocycloalkyl. In embodiments, R¹⁵ is independently substituted or unsubstituted aryl. In embodiments, R¹⁵ is independently unsubstituted phenyl. In embodiments, R¹⁵ is independently substituted or unsubstituted heteroaryl. In embodiments, R¹⁵ is independently unsubstituted heteroaryl. In embodiments, R¹⁵ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹⁵ is independently substituted or unsubstituted C₁-C₆ alkyl. In embodiments, R¹⁵ is independently unsubstituted C₁-C₆ alkyl. In embodiments, R¹⁵ is independently unsubstituted methyl. In embodiments, R¹⁵ is independently unsubstituted ethyl. In embodiments, R¹⁵ is independently unsubstituted propyl. In embodiments, R¹⁵ is independently substituted or unsubstituted 2 to 6 membered heteroalkyl. In embodiments, R¹⁵ is independently unsubstituted 2 to 6 membered heteroalkyl. In embodiments, R¹⁵ is independently substituted or unsubstituted C₃-C₆ cycloalkyl. In embodiments, R¹⁵ is independently unsubstituted C₃-C₆ cycloalkyl. In embodiments, R¹⁵ is independently substituted or unsubstituted 3 to 6 membered heterocycloalkyl. In embodiments, R¹⁵ is independently unsubstituted 3 to 6 membered heterocycloalkyl. In embodiments, R¹⁵ is independently substituted or unsubstituted C₆-C₁₀ aryl. In embodiments, R¹⁵ is independently unsubstituted C₆-C₁₀ aryl. In embodiments, R¹⁵ is independently substituted phenyl. In embodiments, R¹⁵ is independently unsubstituted phenyl. In embodiments, R¹⁵ is independently substituted or unsubstituted 5 to 10 membered heteroaryl. In embodiments, R¹⁵ is independently substituted or unsubstituted 5 to 6 membered heteroaryl. In embodiments, R¹⁵ is independently unsubstituted 5 to 10 membered heteroaryl. In embodiments, R¹⁵ is independently unsubstituted 5 to 6 membered heteroaryl.

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, R¹ is independently hydrogen, substituted or unsubstituted C₁-C₄ alkyl, or substituted or unsubstituted 2 to 4 membered heteroalkyl. In embodiments, R¹ is independently hydrogen. In embodiments, R¹ is independently substituted or unsubstituted C₁-C₄ alkyl. In embodiments, R¹ is independently substituted or unsubstituted 2 to 4 membered heteroalkyl. In embodiments, R¹ is independently unsubstituted C₁-C₄ alkyl. In embodiments, R¹ is independently unsubstituted 2 to 4 membered heteroalkyl. In embodiments, R¹ is independently substituted or unsubstituted C₁-C₆ alkyl or substituted or unsubstituted 2 to 6 membered heteroalkyl. In embodiments, R¹ is independently —Cl. In embodiments, R¹ is independently —Br. In embodiments, R¹ is independently —F. In embodiments, R¹ is independently —I. In embodiments, R¹ is independently —CH₃. In embodiments, R¹ is independently —CCl₃. In embodiments, R¹ is independently —CBr₃. In embodiments, R¹ is independently —CF₃. In embodiments, R¹ is independently —Cl₃. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CH₂C1. In embodiments, R¹ is independently —CH₂Br. In embodiments, R¹ is independently —CH₂F. In embodiments, R¹ is independently —CH₂I. In embodiments, R¹ is independently —CN. In embodiments, R¹ is independently —OCH₃. In embodiments, R¹ is independently —NH₂. In embodiments, R¹ is independently —COOH. In embodiments, R¹ is independently —COCH₃. In embodiments, R¹ is independently —CONH₂. In embodiments, R¹ is independently —OCCl₃. In embodiments, R¹ is independently —OCF₃. In embodiments, R¹ is independently —OCBr₃. In embodiments, R¹ is independently —OCI₃. In embodiments, R¹ is independently —OCHCl₂. In embodiments, R¹ is independently —OCHBr₂. In embodiments, R¹ is independently —OCHI₂. In embodiments, R¹ is independently —OCHF₂. In embodiments, R¹ is independently —OCH₂Cl. In embodiments, R¹ is independently —OCH₂Br. In embodiments, R¹ is independently —OCH₂I. In embodiments, R¹ is independently —OCH₂F. In embodiments, R¹ is independently unsubstituted methyl. In embodiments, R¹ is independently —OCH₃. In embodiments, R¹ is independently —OCH₂CH₃. In embodiments, R¹ is independently —OCH(CH₃)₂. In embodiments, R¹ is independently —OC(CH₃)₃. In embodiments, R¹ is independently —CH₃. In embodiments, R¹ is independently —CH₂CH₃. In embodiments, R¹ is independently —CH(CH₃)₂. In embodiments, R¹ is independently —C(CH₃)₃. In embodiments, R¹ is independently —C(O)CH₃. In embodiments, R¹ is independently —C(O)CH₂CH₃. In embodiments, R¹ is independently —C(O)CH(CH₃)₂. In embodiments, R¹ is independently —C(O)C(CH₃)₃.

In embodiments, R¹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is independently substituted or unsubstituted alkyl. In embodiments, R¹ is independently unsubstituted alkyl. In embodiments, R¹ is independently unsubstituted methyl. In embodiments, R¹ is independently unsubstituted ethyl. In embodiments, R¹ is independently unsubstituted propyl. In embodiments, R¹ is independently substituted or unsubstituted heteroalkyl. In embodiments, R¹ is independently unsubstituted heteroalkyl. In embodiments, R¹ is independently substituted or unsubstituted cycloalkyl. In embodiments, R¹ is independently unsubstituted cycloalkyl. In embodiments, R¹ is independently substituted or unsubstituted heterocycloalkyl. In embodiments, R¹ is independently unsubstituted heterocycloalkyl. In embodiments, R¹ is independently substituted or unsubstituted aryl. In embodiments, R¹ is independently unsubstituted phenyl. In embodiments, R¹ is independently substituted or unsubstituted heteroaryl. In embodiments, R¹ is independently unsubstituted heteroaryl. In embodiments, R¹ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R¹ is independently substituted or unsubstituted C₁-C₆ alkyl. In embodiments, R¹ is independently unsubstituted C₁-C₆ alkyl. In embodiments, R¹ is independently unsubstituted methyl. In embodiments, R¹ is independently unsubstituted ethyl. In embodiments, R¹ is independently unsubstituted propyl. In embodiments, R¹ is independently substituted or unsubstituted 2 to 6 membered heteroalkyl. In embodiments, R¹ is independently unsubstituted 2 to 6 membered heteroalkyl. In embodiments, R¹ is independently substituted or unsubstituted C₃-C₆ cycloalkyl. In embodiments, R¹ is independently unsubstituted C₃-C₆ cycloalkyl. In embodiments, R¹ is independently substituted or unsubstituted 3 to 6 membered heterocycloalkyl. In embodiments, R¹ is independently unsubstituted 3 to 6 membered heterocycloalkyl. In embodiments, R¹ is independently substituted or unsubstituted C₆-C₁₀ aryl. In embodiments, R¹ is independently unsubstituted C₆-C₁₀ aryl. In embodiments, R¹ is independently substituted phenyl. In embodiments, R¹ is independently unsubstituted phenyl. In embodiments, R¹ is independently substituted or unsubstituted 5 to 10 membered heteroaryl. In embodiments, R¹ is independently substituted or unsubstituted 5 to 6 membered heteroaryl. In embodiments, R¹ is independently unsubstituted 5 to 10 membered heteroaryl. In embodiments, R¹ is independently unsubstituted 5 to 6 membered heteroaryl.

In embodiments, R² is a 14-3-3 C38 non-covalent binding moiety. In embodiments, R² is a 14-3-3 C38 covalent binding moiety. In embodiments, R² is a 14-3-3β N40 binding moiety (14-3-3beta). In embodiments, R² is a 14-3-3ε V39 binding moiety (14-3-3epsilon). In embodiments, R² is a 14-3-3η N39 binding moiety (14-3-3eta). In embodiments, R² is a 14-3-3γ N39 binding moiety (14-3-3gamma). In embodiments, R² is a 14-3-3σ C38 binding moiety (14-3-3sigma). In embodiments, R² is a 14-3-3τ N38 binding moiety (14-3-3tau). In embodiments, R² is a 14-3-3 N38 binding moiety (14-3-3zeta).

In embodiments, R² is a 14-3-3σ C38 non-covalent binding moiety. In embodiments, R² is a 14-3-3σ C38 covalent binding moiety.

In embodiments, R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R²C, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 12 membered, 5 to 10 membered, 5 to 9 membered, or 5 to 6 membered).

X² is independently —F, —Cl, —Br, or —I;

In embodiments, X² is independently —F. In embodiments, X² is independently —Cl. In embodiments, X² is independently —Br. In embodiments, X² is independently —I.

n2 is independently an integer from 0 to 4; and

In embodiments, n2 is independently 0. In embodiments, n2 is independently 1. In embodiments, n2 is independently 2. In embodiments, n2 is independently 3. In embodiments, n2 is independently 4.

m2 and v2 are independently 1 or 2.

In embodiments, m2 is independently 1. In embodiments, m2 is independently 2. In embodiments, v2 is independently 1. In embodiments, v2 is independently 2.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{2A} and R^{2B} substituents bonded to the same nitrogen atom are joined is substituted, it is substituted with at least one substituent group. In embodiments, when the ring formed when R^{2A} and R^{2B} substituents bonded to the same nitrogen atom are joined is substituted, it is substituted with at least one size-limited substituent group. In embodiments, when the ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is independently hydrogen. In embodiments, R^{2B} is independently hydrogen. In embodiments, R^{2C} is independently hydrogen. In embodiments, R^{2D} is independently hydrogen.

In embodiments, R^{2A} is independently unsubstituted C₁-C₄ alkyl. In embodiments, R^{2B} is independently unsubstituted C₁-C₄ alkyl. In embodiments, R^{2C} is independently unsubstituted C₁-C₄ alkyl. In embodiments, R^{2D} is independently unsubstituted C₁-C₄ alkyl.

In embodiments, R² is independently hydrogen, substituted or unsubstituted C₁-C₄ alkyl, or substituted or unsubstituted 2 to 4 membered heteroalkyl. In embodiments, R² is independently hydrogen. In embodiments, R² is independently substituted or unsubstituted C₁-C₄ alkyl. In embodiments, R² is independently substituted or unsubstituted 2 to 4 membered heteroalkyl. In embodiments, R² is independently unsubstituted C₁-C₄ alkyl. In embodiments, R² is independently unsubstituted 2 to 4 membered heteroalkyl. In embodiments, R² is independently substituted or unsubstituted C₁-C₆ alkyl or substituted or unsubstituted 2 to 6 membered heteroalkyl. In embodiments, R² is independently —Cl. In embodiments, R² is independently —Br. In embodiments, R² is independently —F. In embodiments, R² is independently —I. In embodiments, R² is independently —CH₃. In embodiments, R² is independently —CCl₃. In embodiments, R² is independently —CBr₃. In embodiments, R² is independently —CF₃. In embodiments, R² is independently —CI₃. In embodiments, R² is independently —CHCl₂. In embodiments, R² is independently —CHBr₂. In embodiments, R² is independently —CHF₂. In embodiments, R² is independently —CHI₂. In embodiments, R² is independently —CH₂C1. In embodiments, R² is independently —CH₂Br. In embodiments, R² is independently —CH₂F. In embodiments, R² is independently —CH₂I. In embodiments, R² is independently —CN. In embodiments, R² is independently —OCH₃. In embodiments, R² is independently —NH₂. In embodiments, R² is independently —COOH. In embodiments, R² is independently —COCH₃. In embodiments, R² is independently —CONH₂. In embodiments, R² is independently —OCCl₃. In embodiments, R² is independently —OCF₃. In embodiments, R² is independently —OCBr₃. In embodiments, R² is independently —OCI₃. In embodiments, R² is independently —OCHCl₂. In embodiments, R² is independently —OCHBr₂. In embodiments, R² is independently —OCHI₂. In embodiments, R² is independently —OCHF₂. In embodiments, R² is independently —OCH₂Cl. In embodiments, R² is independently —OCH₂Br. In embodiments, R² is independently —OCH₂I. In embodiments, R² is independently —OCH₂F. In embodiments, R² is independently unsubstituted methyl. In embodiments, R² is independently —OCH₃. In embodiments, R² is independently —OCH₂CH₃. In embodiments, R² is independently —OCH(CH₃)₂. In embodiments, R² is independently —OC(CH₃)₃. In embodiments, R² is independently —CH₃. In embodiments, R² is independently —CH₂CH₃. In embodiments, R² is independently —CH(CH₃)₂. In embodiments, R² is independently —C(CH₃)₃. In embodiments, R² is independently —C(O)CH₃. In embodiments, R² is independently —C(O)CH₂CH₃. In embodiments, R² is independently —C(O)CH(CH₃)₂. In embodiments, R² is independently —C(O)C(CH₃)₃.

In embodiments, R² is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is independently substituted or unsubstituted alkyl. In embodiments, R² is independently unsubstituted alkyl. In embodiments, R² is independently unsubstituted methyl. In embodiments, R² is independently unsubstituted ethyl. In embodiments, R² is independently unsubstituted propyl. In embodiments, R² is independently substituted or unsubstituted heteroalkyl. In embodiments, R² is independently unsubstituted heteroalkyl. In embodiments, R² is independently substituted or unsubstituted cycloalkyl. In embodiments, R² is independently unsubstituted cycloalkyl. In embodiments, R² is independently substituted or unsubstituted heterocycloalkyl. In embodiments, R² is independently unsubstituted heterocycloalkyl. In embodiments, R² is independently substituted or unsubstituted aryl. In embodiments, R² is independently unsubstituted phenyl. In embodiments, R² is independently substituted or unsubstituted heteroaryl. In embodiments, R² is independently unsubstituted heteroaryl. In embodiments, R² is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R² is independently substituted or unsubstituted C₁-C₆ alkyl. In embodiments, R² is independently unsubstituted C₁-C₆ alkyl. In embodiments, R² is independently unsubstituted methyl. In embodiments, R² is independently unsubstituted ethyl. In embodiments, R² is independently unsubstituted propyl. In embodiments, R² is independently substituted or unsubstituted 2 to 6 membered heteroalkyl. In embodiments, R² is independently unsubstituted 2 to 6 membered heteroalkyl. In embodiments, R² is independently substituted or unsubstituted C₃-C₆ cycloalkyl. In embodiments, R² is independently unsubstituted C₃-C₆ cycloalkyl. In embodiments, R² is independently substituted or unsubstituted 3 to 6 membered heterocycloalkyl. In embodiments, R² is independently unsubstituted 3 to 6 membered heterocycloalkyl. In embodiments, R² is independently substituted or unsubstituted C₆-C₁₀ aryl. In embodiments, R² is independently unsubstituted C₆-C₁₀ aryl. In embodiments, R² is independently substituted phenyl. In embodiments, R² is independently unsubstituted phenyl. In embodiments, R² is independently substituted or unsubstituted 5 to 10 membered heteroaryl. In embodiments, R² is independently substituted or unsubstituted 5 to 6 membered heteroaryl. In embodiments, R² is independently unsubstituted 5 to 10 membered heteroaryl. In embodiments, R² is independently unsubstituted 5 to 6 membered heteroaryl.

In embodiments, R² is -L^{A2}-L^{2B}-E2.

L^{2A} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L^{2A} is independently a bond.

In embodiments, L^{2A} is independently —S(O)₂—. In embodiments, L^{2A} is independently —NH—. In embodiments, L^{2A} is independently —O—. In embodiments, L^{2A} is independently —S—. In embodiments, L^{2A} is independently —C(O)—. In embodiments, L^{2A} is independently —NHS(O)₂—. In embodiments, L^{2A} is independently —S(O)₂NH—. In embodiments, L^{2A} is independently —C(O)NH—. In embodiments, L^{2A} is independently —NHC(O)—. In embodiments, L^{2A} is independently —NHC(O)NH—. In embodiments, L^{2A} is independently —C(O)O—. In embodiments, L^{2A} is independently —OC(O)—.

In embodiments, L^{2A} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{2A} is independently substituted or unsubstituted alkylene. In embodiments, L^{2A} is independently unsubstituted alkylene. In embodiments, L^{2A} is independently unsubstituted methylene. In embodiments, L^{2A} is independently unsubstituted ethylene. In embodiments, L^{2A} is independently unsubstituted propylene. In embodiments, L^{2A} is independently substituted or unsubstituted heteroalkylene. In embodiments, L^{2A} is independently unsubstituted heteroalkylene. In embodiments, L^{2A} is independently substituted or unsubstituted cycloalkylene. In embodiments, L^{2A} is independently unsubstituted cycloalkylene. In embodiments, L^{2A} is independently substituted or unsubstituted heterocycloalkylene. In embodiments, L^{2A} is independently unsubstituted heterocycloalkylene. In embodiments, L^{2A} is independently substituted or unsubstituted arylene. In embodiments, L^{2A} is independently unsubstituted phenylene. In embodiments, L^{2A} is independently substituted or unsubstituted heteroarylene. In embodiments, L^{2A} is independently unsubstituted heteroarylene. In embodiments, L^{2A} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{2A} is independently substituted or unsubstituted C₁-C₆ alkylene. In embodiments, L^{2A} is independently unsubstituted C₁-C₆ alkylene. In embodiments, L^{2A} is independently unsubstituted methylene. In embodiments, L^{2A} is independently unsubstituted ethylene. In embodiments, L^{2A} is independently unsubstituted propylene. In embodiments, L^{2A} is independently substituted or unsubstituted 2 to 6 membered heteroalkylene. In embodiments, L^{2A} is independently unsubstituted 2 to 6 membered heteroalkylene. In embodiments, L^{2A} is independently substituted or unsubstituted C₃-C₆ cycloalkylene. In embodiments, L^{2A} is independently unsubstituted C₃-C₆ cycloalkylene. In embodiments, L^{2A} is independently substituted or unsubstituted 3 to 6 membered heterocycloalkylene. In embodiments, L^{2A} is independently unsubstituted 3 to 6 membered heterocycloalkylene. In embodiments, L^{2A} is independently substituted or unsubstituted C₆-C₁₀ arylene. In embodiments, L^{2A} is independently unsubstituted C₆-C₁₀ arylene. In embodiments, L^{2A} is independently substituted phenylene. In embodiments, L^{2A} is independently unsubstituted phenylene. In embodiments, L^{2A} is independently substituted or unsubstituted 5 to 10 membered heteroarylene. In embodiments, L^{2A} is independently substituted or unsubstituted 5 to 6 membered heteroarylene. In embodiments, L^{2A} is independently unsubstituted 5 to 10 membered heteroarylene. In embodiments, L^{2A} is independently unsubstituted 5 to 6 membered heteroarylene.

In embodiments, a substituted L^{2A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2A} is substituted with a plurality of groups selected from substituent groups, size-limited substituent groups, and lower substituent groups; each substituent group, size-limited substituent group, and/or lower substituent group may optionally be different. In embodiments, when L^{2A} is substituted, it is substituted with at least one substituent group. In embodiments, when L^{2A} is substituted, it is substituted with at least one size-limited substituent group. In embodiments, when L^{2A} is substituted, it is substituted with at least one lower substituent group.

L^{2B} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L^{2B} is independently a bond, —NH—, —C(O)NH—, —NHC(O)—, —NHC(O)NH—, substituted or unsubstituted heteroalkylene (e.g., 2 to 8 membered, 2 to 6 membered, 4 to 6 membered, 2 to 3 membered, or 4 to 5 membered), substituted or unsubstituted heterocycloalkylene (e.g., 3 to 8 membered, 3 to 6 membered, 4 to 6 membered, 4 to 5 membered, or 5 to 6 membered), or substituted or unsubstituted heteroarylene (e.g., 5 to 12 membered, 5 to 10 membered, 5 to 9 membered, or 5 to 6 membered).

In embodiments, L^{2B} is independently a bond.

In embodiments, L^{2B} is independently —NH—. In embodiments, L^{2B} is independently —C(O)NH—. In embodiments, L^{2B} is independently —NHC(O)—. In embodiments, L^{2B} is independently —NHC(O)NH—.

In embodiments, L^{2B} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{2B} is independently substituted or unsubstituted alkylene. In embodiments, L^{2B} is independently unsubstituted alkylene. In embodiments, L^{2B} is independently unsubstituted methylene. In embodiments, L^{2B} is independently unsubstituted ethylene. In embodiments, L^{2B} is independently unsubstituted propylene. In embodiments, L^{2B} is independently substituted or unsubstituted heteroalkylene. In embodiments, L^{2B} is independently unsubstituted heteroalkylene. In embodiments, L^{2B} is independently substituted or unsubstituted cycloalkylene. In embodiments, L^{2B} is independently unsubstituted cycloalkylene. In embodiments, L^{2B} is independently substituted or unsubstituted heterocycloalkylene. In embodiments, L^{2B} is independently unsubstituted heterocycloalkylene. In embodiments, L^{2B} is independently substituted or unsubstituted arylene. In embodiments, L^{2B} is independently unsubstituted phenylene. In embodiments, L^{2B} is independently substituted or unsubstituted heteroarylene. In embodiments, L^{2B} is independently unsubstituted heteroarylene. In embodiments, L^{2B} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{2B} is independently substituted or unsubstituted C₁-C₆ alkylene. In embodiments, L^{2B} is independently unsubstituted C₁-C₆ alkylene. In embodiments, L^{2B} is independently unsubstituted methylene. In embodiments, L^{2B} is independently unsubstituted ethylene. In embodiments, L^{2B} is independently unsubstituted propylene. In embodiments, L^{2B} is independently substituted or unsubstituted 2 to 6 membered heteroalkylene. In embodiments, L^{2B} is independently unsubstituted 2 to 6 membered heteroalkylene. In embodiments, L^{2B} is independently substituted or unsubstituted C₃-C₆ cycloalkylene. In embodiments, L^{2B} is independently unsubstituted C₃-C₆ cycloalkylene. In embodiments, L^{2B} is independently substituted or unsubstituted 3 to 6 membered heterocycloalkylene. In embodiments, L^{2B} is independently unsubstituted 3 to 6 membered heterocycloalkylene. In embodiments, L^{2B} is independently substituted or unsubstituted C₆-C₁₀ arylene. In embodiments, L^{2B} is independently unsubstituted C₆-C₁₀ arylene. In embodiments, L^{2B} is independently substituted phenylene. In embodiments, L^{2B} is independently unsubstituted phenylene. In embodiments, L^{2B} is independently substituted or unsubstituted 5 to 10 membered heteroarylene. In embodiments, L^{2B} is independently substituted or unsubstituted 5 to 6 membered heteroarylene. In embodiments, L^{2B} is independently unsubstituted 5 to 10 membered heteroarylene. In embodiments, L^{2B} is independently unsubstituted 5 to 6 membered heteroarylene.

In embodiments, a substituted L^{2B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{2B} is substituted with a plurality of groups selected from substituent groups, size-limited substituent groups, and lower substituent groups; each substituent group, size-limited substituent group, and/or lower substituent group may optionally be different. In embodiments, when L^{2B} is substituted, it is substituted with at least one substituent group. In embodiments, when L^{2B} is substituted, it is substituted with at least one size-limited substituent group. In embodiments, when L^{2B} is substituted, it is substituted with at least one lower substituent group.

E2 is a covalent cysteine modifier moiety. In embodiments, E2 is a 14-3-3 C38 covalent binding moiety. In embodiments, E2 is —SH, —SSR²⁶,

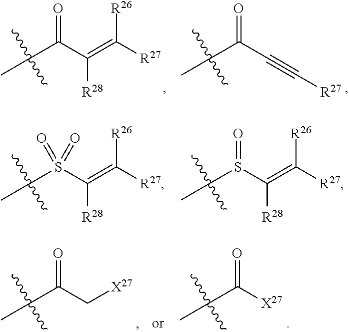

R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, R²⁶ is independently hydrogen, substituted or unsubstituted C₁-C₄ alkyl, or substituted or unsubstituted 2 to 4 membered heteroalkyl. In embodiments, R²⁶ is independently hydrogen. In embodiments, R²⁶ is independently substituted or unsubstituted C₁-C₄ alkyl. In embodiments, R²⁶ is independently substituted or unsubstituted 2 to 4 membered heteroalkyl. In embodiments, R²⁶ is independently unsubstituted C₁-C₄ alkyl. In embodiments, R²⁶ is independently unsubstituted 2 to 4 membered heteroalkyl. In embodiments, R²⁶ is independently substituted or unsubstituted C₁-C₆ alkyl or substituted or unsubstituted 2 to 6 membered heteroalkyl. In embodiments, R²⁶ is independently —Cl. In embodiments, R²⁶ is independently —Br. In embodiments, R²⁶ is independently —F. In embodiments, R²⁶ is independently —I. In embodiments, R²⁶ is independently —CH₃. In embodiments, R²⁶ is independently —CCl₃. In embodiments, R²⁶ is independently —CBr₃. In embodiments, R²⁶ is independently —CF₃. In embodiments, R²⁶ is independently —CI₃. In embodiments, R²⁶ is independently —CHCl₂. In embodiments, R²⁶ is independently —CHBr₂. In embodiments, R²⁶ is independently —CHF₂. In embodiments, R²⁶ is independently —CHI₂. In embodiments, R²⁶ is independently —CH₂C1. In embodiments, R²⁶ is independently —CH₂Br. In embodiments, R²⁶ is independently —CH₂F. In embodiments, R²⁶ is independently —CH₂I. In embodiments, R²⁶ is independently —CN. In embodiments, R²⁶ is independently —OCH₃. In embodiments, R²⁶ is independently —NH₂. In embodiments, R²⁶ is independently —COOH. In embodiments, R²⁶ is independently —COCH₃. In embodiments, R²⁶ is independently —CONH₂. In embodiments, R²⁶ is independently —OCCl₃. In embodiments, R²⁶ is independently —OCF₃. In embodiments, R²⁶ is independently —OCBr₃. In embodiments, R²⁶ is independently —OCI₃. In embodiments, R²⁶ is independently —OCHCl₂. In embodiments, R²⁶ is independently —OCHBr₂. In embodiments, R²⁶ is independently —OCHI₂. In embodiments, R²⁶ is independently —OCHF₂. In embodiments, R²⁶ is independently —OCH₂Cl. In embodiments, R²⁶ is independently —OCH₂Br. In embodiments, R²⁶ is independently —OCH₂I. In embodiments, R²⁶ is independently —OCH₂F. In embodiments, R²⁶ is independently unsubstituted methyl. In embodiments, R²⁶ is independently —OCH₃. In embodiments, R²⁶ is independently —OCH₂CH₃. In embodiments, R²⁶ is independently —OCH(CH₃)₂. In embodiments, R²⁶ is independently —OC(CH₃)₃. In embodiments, R²⁶ is independently —CH₃. In embodiments, R²⁶ is independently —CH₂CH₃. In embodiments, R²⁶ is independently —CH(CH₃)₂. In embodiments, R²⁶ is independently —C(CH₃)₃. In embodiments, R²⁶ is independently —C(O)CH₃. In embodiments, R²⁶ is independently —C(O)CH₂CH₃. In embodiments, R²⁶ is independently —C(O)CH(CH₃)₂. In embodiments, R²⁶ is independently —C(O)C(CH₃)₃.

In embodiments, R²⁶ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁶ is independently substituted or unsubstituted alkyl. In embodiments, R²⁶ is independently unsubstituted alkyl. In embodiments, R²⁶ is independently unsubstituted methyl. In embodiments, R²⁶ is independently unsubstituted ethyl. In embodiments, R²⁶ is independently unsubstituted propyl. In embodiments, R²⁶ is independently substituted or unsubstituted heteroalkyl. In embodiments, R²⁶ is independently unsubstituted heteroalkyl. In embodiments, R²⁶ is independently substituted or unsubstituted cycloalkyl. In embodiments, R²⁶ is independently unsubstituted cycloalkyl. In embodiments, R²⁶ is independently substituted or unsubstituted heterocycloalkyl. In embodiments, R²⁶ is independently unsubstituted heterocycloalkyl. In embodiments, R²⁶ is independently substituted or unsubstituted aryl. In embodiments, R²⁶ is independently unsubstituted phenyl. In embodiments, R²⁶ is independently substituted or unsubstituted heteroaryl. In embodiments, R²⁶ is independently unsubstituted heteroaryl. In embodiments, R²⁶ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R²⁶ is independently substituted or unsubstituted C₁-C₆ alkyl. In embodiments, R²⁶ is independently unsubstituted C₁-C₆ alkyl. In embodiments, R²⁶ is independently unsubstituted methyl. In embodiments, R²⁶ is independently unsubstituted ethyl. In embodiments, R²⁶ is independently unsubstituted propyl. In embodiments, R²⁶ is independently substituted or unsubstituted 2 to 6 membered heteroalkyl. In embodiments, R²⁶ is independently unsubstituted 2 to 6 membered heteroalkyl. In embodiments, R²⁶ is independently substituted or unsubstituted C₃-C₆ cycloalkyl. In embodiments, R²⁶ is independently unsubstituted C₃-C₆ cycloalkyl. In embodiments, R²⁶ is independently substituted or unsubstituted 3 to 6 membered heterocycloalkyl. In embodiments, R²⁶ is independently unsubstituted 3 to 6 membered heterocycloalkyl. In embodiments, R²⁶ is independently substituted or unsubstituted C₆-C₁₀ aryl. In embodiments, R²⁶ is independently unsubstituted C₆-C₁₀ aryl. In embodiments, R²⁶ is independently substituted phenyl. In embodiments, R²⁶ is independently unsubstituted phenyl. In embodiments, R²⁶ is independently substituted or unsubstituted 5 to 10 membered heteroaryl. In embodiments, R²⁶ is independently substituted or unsubstituted 5 to 6 membered heteroaryl. In embodiments, R²⁶ is independently unsubstituted 5 to 10 membered heteroaryl. In embodiments, R²⁶ is independently unsubstituted 5 to 6 membered heteroaryl.

In embodiments, a substituted R²⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁶ is substituted with a plurality of groups selected from substituent groups, size-limited substituent groups, and lower substituent groups; each substituent group, size-limited substituent group, and/or lower substituent group may optionally be different. In embodiments, when R²⁶ is substituted, it is substituted with at least one substituent group. In embodiments, when R²⁶ is substituted, it is substituted with at least one size-limited substituent group. In embodiments, when R²⁶ is substituted, it is substituted with at least one lower substituent group.

In embodiments, R²⁷ is independently hydrogen, substituted or unsubstituted C₁-C₄ alkyl, or substituted or unsubstituted 2 to 4 membered heteroalkyl. In embodiments, R²⁷ is independently hydrogen. In embodiments, R²⁷ is independently substituted or unsubstituted C₁-C₄ alkyl. In embodiments, R²⁷ is independently substituted or unsubstituted 2 to 4 membered heteroalkyl. In embodiments, R²⁷ is independently unsubstituted C₁-C₄ alkyl. In embodiments, R²⁷ is independently unsubstituted 2 to 4 membered heteroalkyl. In embodiments, R²⁷ is independently substituted or unsubstituted C₁-C₆ alkyl or substituted or unsubstituted 2 to 6 membered heteroalkyl. In embodiments, R²⁷ is independently —Cl. In embodiments, R²⁷ is independently —Br. In embodiments, R²⁷ is independently —F. In embodiments, R²⁷ is independently —I. In embodiments, R²⁷ is independently —CH₃. In embodiments, R²⁷ is independently —CCl₃. In embodiments, R²⁷ is independently —CBr₃. In embodiments, R²⁷ is independently —CF₃. In embodiments, R²⁷ is independently —CI₃. In embodiments, R²⁷ is independently —CHCl₂. In embodiments, R²⁷ is independently —CHBr₂. In embodiments, R²⁷ is independently —CHF₂. In embodiments, R²⁷ is independently —CHI₂. In embodiments, R²⁷ is independently —CH₂C1. In embodiments, R²⁷ is independently —CH₂Br. In embodiments, R²⁷ is independently —CH₂F. In embodiments, R²⁷ is independently —CH₂I. In embodiments, R²⁷ is independently —CN. In embodiments, R²⁷ is independently —OCH₃. In embodiments, R²⁷ is independently —NH₂. In embodiments, R²⁷ is independently —COOH. In embodiments, R²⁷ is independently —COCH₃. In embodiments, R²⁷ is independently —CONH₂. In embodiments, R²⁷ is independently —OCCl₃. In embodiments, R²⁷ is independently —OCF₃. In embodiments, R²⁷ is independently —OCBr₃. In embodiments, R²⁷ is independently —OCI₃. In embodiments, R²⁷ is independently —OCHCl₂. In embodiments, R²⁷ is independently —OCHBr₂. In embodiments, R²⁷ is independently —OCHI₂. In embodiments, R²⁷ is independently —OCHF₂. In embodiments, R²⁷ is independently —OCH₂Cl. In embodiments, R²⁷ is independently —OCH₂Br. In embodiments, R²⁷ is independently —OCH₂I. In embodiments, R²⁷ is independently —OCH₂F. In embodiments, R²⁷ is independently unsubstituted methyl. In embodiments, R²⁷ is independently —OCH₃. In embodiments, R²⁷ is independently —OCH₂CH₃. In embodiments, R²⁷ is independently —OCH(CH₃)₂. In embodiments, R²⁷ is independently —OC(CH₃)₃. In embodiments, R²⁷ is independently —CH₃. In embodiments, R²⁷ is independently —CH₂CH₃. In embodiments, R²⁷ is independently —CH(CH₃)₂. In embodiments, R²⁷ is independently —C(CH₃)₃. In embodiments, R²⁷ is independently —C(O)CH₃. In embodiments, R²⁷ is independently —C(O)CH₂CH₃. In embodiments, R²⁷ is independently —C(O)CH(CH₃)₂. In embodiments, R²⁷ is independently —C(O)C(CH₃)₃.

In embodiments, R²⁷ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁷ is independently substituted or unsubstituted alkyl. In embodiments, R²⁷ is independently unsubstituted alkyl. In embodiments, R²⁷ is independently unsubstituted methyl. In embodiments, R²⁷ is independently unsubstituted ethyl. In embodiments, R²⁷ is independently unsubstituted propyl. In embodiments, R²⁷ is independently substituted or unsubstituted heteroalkyl. In embodiments, R²⁷ is independently unsubstituted heteroalkyl. In embodiments, R²⁷ is independently substituted or unsubstituted cycloalkyl. In embodiments, R²⁷ is independently unsubstituted cycloalkyl. In embodiments, R²⁷ is independently substituted or unsubstituted heterocycloalkyl. In embodiments, R²⁷ is independently unsubstituted heterocycloalkyl. In embodiments, R²⁷ is independently substituted or unsubstituted aryl. In embodiments, R²⁷ is independently unsubstituted phenyl. In embodiments, R²⁷ is independently substituted or unsubstituted heteroaryl. In embodiments, R²⁷ is independently unsubstituted heteroaryl. In embodiments, R²⁷ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R²⁷ is independently substituted or unsubstituted C₁-C₆ alkyl. In embodiments, R²⁷ is independently unsubstituted C₁-C₆ alkyl. In embodiments, R²⁷ is independently unsubstituted methyl. In embodiments, R²⁷ is independently unsubstituted ethyl. In embodiments, R²⁷ is independently unsubstituted propyl. In embodiments, R²⁷ is independently substituted or unsubstituted 2 to 6 membered heteroalkyl. In embodiments, R²⁷ is independently unsubstituted 2 to 6 membered heteroalkyl. In embodiments, R²⁷ is independently substituted or unsubstituted C₃-C₆ cycloalkyl. In embodiments, R²⁷ is independently unsubstituted C₃-C₆ cycloalkyl. In embodiments, R²⁷ is independently substituted or unsubstituted 3 to 6 membered heterocycloalkyl. In embodiments, R²⁷ is independently unsubstituted 3 to 6 membered heterocycloalkyl. In embodiments, R²⁷ is independently substituted or unsubstituted C₆-C₁₀ aryl. In embodiments, R²⁷ is independently unsubstituted C₆-C₁₀ aryl. In embodiments, R²⁷ is independently substituted phenyl. In embodiments, R²⁷ is independently unsubstituted phenyl. In embodiments, R²⁷ is independently substituted or unsubstituted 5 to 10 membered heteroaryl. In embodiments, R²⁷ is independently substituted or unsubstituted 5 to 6 membered heteroaryl. In embodiments, R²⁷ is independently unsubstituted 5 to 10 membered heteroaryl. In embodiments, R²⁷ is independently unsubstituted 5 to 6 membered heteroaryl.

In embodiments, a substituted R²⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁷ is substituted with a plurality of groups selected from substituent groups, size-limited substituent groups, and lower substituent groups; each substituent group, size-limited substituent group, and/or lower substituent group may optionally be different. In embodiments, when R²⁷ is substituted, it is substituted with at least one substituent group. In embodiments, when R²⁷ is substituted, it is substituted with at least one size-limited substituent group. In embodiments, when R²⁷ is substituted, it is substituted with at least one lower substituent group.

In embodiments, R²⁸ is independently hydrogen, substituted or unsubstituted C₁-C₄ alkyl, or substituted or unsubstituted 2 to 4 membered heteroalkyl. In embodiments, R²⁸ is independently hydrogen. In embodiments, R²⁸ is independently substituted or unsubstituted C₁-C₄ alkyl. In embodiments, R²⁸ is independently substituted or unsubstituted 2 to 4 membered heteroalkyl. In embodiments, R²⁸ is independently unsubstituted C₁-C₄ alkyl. In embodiments, R²⁸ is independently unsubstituted 2 to 4 membered heteroalkyl. In embodiments, R²⁸ is independently substituted or unsubstituted C₁-C₆ alkyl or substituted or unsubstituted 2 to 6 membered heteroalkyl. In embodiments, R²⁸ is independently —Cl. In embodiments, R²⁸ is independently —Br. In embodiments, R²⁸ is independently —F. In embodiments, R²⁸ is independently —I. In embodiments, R²⁸ is independently —CH₃. In embodiments, R²⁸ is independently —CCl₃. In embodiments, R²⁸ is independently —CBr₃. In embodiments, R²⁸ is independently —CF₃. In embodiments, R²⁸ is independently —CI₃. In embodiments, R²⁸ is independently —CHCl₂. In embodiments, R²⁸ is independently —CHBr₂. In embodiments, R²⁸ is independently —CHF₂. In embodiments, R²⁸ is independently —CHI₂. In embodiments, R²⁸ is independently —CH₂C1. In embodiments, R²⁸ is independently —CH₂Br. In embodiments, R²⁸ is independently —CH₂F. In embodiments, R²⁸ is independently —CH₂I. In embodiments, R²⁸ is independently —CN. In embodiments, R²⁸ is independently —OCH₃. In embodiments, R²⁸ is independently —NH₂. In embodiments, R²⁸ is independently —COOH. In embodiments, R²⁸ is independently —COCH₃. In embodiments, R²⁸ is independently —CONH₂. In embodiments, R²⁸ is independently —OCCl₃. In embodiments, R²⁸ is independently —OCF₃. In embodiments, R²⁸ is independently —OCBr₃. In embodiments, R²⁸ is independently —OCI₃. In embodiments, R²⁸ is independently —OCHCl₂. In embodiments, R²⁸ is independently —OCHBr₂. In embodiments, R²⁸ is independently —OCHI₂. In embodiments, R²⁸ is independently —OCHF₂. In embodiments, R²⁸ is independently —OCH₂Cl. In embodiments, R²⁸ is independently —OCH₂Br. In embodiments, R²⁸ is independently —OCH₂I. In embodiments, R²⁸ is independently —OCH₂F. In embodiments, R²⁸ is independently unsubstituted methyl. In embodiments, R²⁸ is independently —OCH₃. In embodiments, R²⁸ is independently —OCH₂CH₃. In embodiments, R²⁸ is independently —OCH(CH₃)₂. In embodiments, R²⁸ is independently —OC(CH₃)₃. In embodiments, R²⁸ is independently —CH₃. In embodiments, R²⁸ is independently —CH₂CH₃. In embodiments, R²⁸ is independently —CH(CH₃)₂. In embodiments, R²⁸ is independently —C(CH₃)₃. In embodiments, R²⁸ is independently —C(O)CH₃. In embodiments, R²⁸ is independently —C(O)CH₂CH₃. In embodiments, R²⁸ is independently —C(O)CH(CH₃)₂. In embodiments, R²⁸ is independently —C(O)C(CH₃)₃.

In embodiments, R²⁸ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁸ is independently substituted or unsubstituted alkyl. In embodiments, R²⁸ is independently unsubstituted alkyl. In embodiments, R²⁸ is independently unsubstituted methyl. In embodiments, R²⁸ is independently unsubstituted ethyl. In embodiments, R²⁸ is independently unsubstituted propyl. In embodiments, R²⁸ is independently substituted or unsubstituted heteroalkyl. In embodiments, R²⁸ is independently unsubstituted heteroalkyl. In embodiments, R²⁸ is independently substituted or unsubstituted cycloalkyl. In embodiments, R²⁸ is independently unsubstituted cycloalkyl. In embodiments, R²⁸ is independently substituted or unsubstituted heterocycloalkyl. In embodiments, R²⁸ is independently unsubstituted heterocycloalkyl. In embodiments, R²⁸ is independently substituted or unsubstituted aryl. In embodiments, R²⁸ is independently unsubstituted phenyl. In embodiments, R²⁸ is independently substituted or unsubstituted heteroaryl. In embodiments, R²⁸ is independently unsubstituted heteroaryl. In embodiments, R²⁸ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R²⁸ is independently substituted or unsubstituted C₁-C₆ alkyl. In embodiments, R²⁸ is independently unsubstituted C₁-C₆ alkyl. In embodiments, R²⁸ is independently unsubstituted methyl. In embodiments, R²⁸ is independently unsubstituted ethyl. In embodiments, R²⁸ is independently unsubstituted propyl. In embodiments, R²⁸ is independently substituted or unsubstituted 2 to 6 membered heteroalkyl. In embodiments, R²⁸ is independently unsubstituted 2 to 6 membered heteroalkyl. In embodiments, R²⁸ is independently substituted or unsubstituted C₃-C₆ cycloalkyl. In embodiments, R²⁸ is independently unsubstituted C₃-C₆ cycloalkyl. In embodiments, R²⁸ is independently substituted or unsubstituted 3 to 6 membered heterocycloalkyl. In embodiments, R²⁸ is independently unsubstituted 3 to 6 membered heterocycloalkyl. In embodiments, R²⁸ is independently substituted or unsubstituted C₆-C₁₀ aryl. In embodiments, R²⁸ is independently unsubstituted C₆-C₁₀ aryl. In embodiments, R²⁸ is independently substituted phenyl. In embodiments, R²⁸ is independently unsubstituted phenyl. In embodiments, R²⁸ is independently substituted or unsubstituted 5 to 10 membered heteroaryl. In embodiments, R²⁸ is independently substituted or unsubstituted 5 to 6 membered heteroaryl. In embodiments, R²⁸ is independently unsubstituted 5 to 10 membered heteroaryl. In embodiments, R²⁸ is independently unsubstituted 5 to 6 membered heteroaryl.

In embodiments, a substituted R²⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁸ is substituted with a plurality of groups selected from substituent groups, size-limited substituent groups, and lower substituent groups; each substituent group, size-limited substituent group, and/or lower substituent group may optionally be different. In embodiments, when R²⁸ is substituted, it is substituted with at least one substituent group. In embodiments, when R²⁸ is substituted, it is substituted with at least one size-limited substituent group. In embodiments, when R²⁸ is substituted, it is substituted with at least one lower substituent group.

X²⁷ is independently —F, —Cl, —Br, or —I.

In embodiments, X²⁷ is independently —F. In embodiments, X²⁷ is independently —Cl. In embodiments, X²⁷ is independently —Br. In embodiments, X²⁷ is independently —I.

In embodiments, E2 is

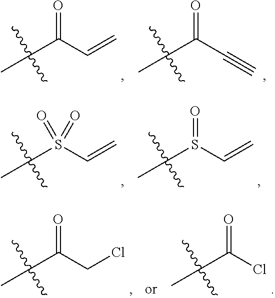

In embodiments, E2 is —SH. In embodiments, E2 is —SSR²⁶. In embodiments, E2 is

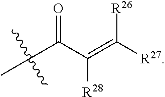

In embodiments, E2 is

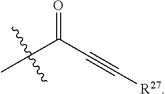

In embodiments, E2 is

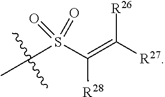

In embodiments, E2 is

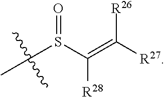

In embodiments, E2 is

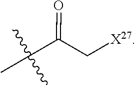

In embodiments, E2 is

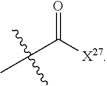

In embodiments, E2 is

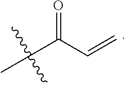 [SMILES: *C(=O)C=C]

In embodiments, E2 is

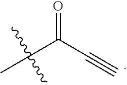 [SMILES: *C(=O)C#C]

In embodiments, E2 is

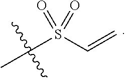 [SMILES: *S(=O)(=O)C=C]

In embodiments, E2 is

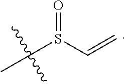 [SMILES: *S(=O)C=C]

In embodiments, E2 is

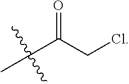 [SMILES: *C(=O)CCl]

In embodiments, E2 is.

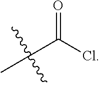 [SMILES: *C(=O)Cl]

In embodiments, E2 is not —SSR²⁶. In embodiments, R² is not —SSR²⁶. In embodiments, E2 is not —SSH. In embodiments, R² is not —SSH. In embodiments, R² does not include —SSR²⁶. In embodiments, R² does not include —SSH. In embodiments, R² does not include a disulfide. In embodiments, E2 is not —SR^{2D}. In embodiments, R² is not —SR^{2D}. In embodiments, R² does not include —SR^{2D}. In embodiments, E2 is not —SH. In embodiments, R² is not —SH. In embodiments, R² does not include —SH. In embodiments, R² does not include a thiol.

In embodiments, E2 is

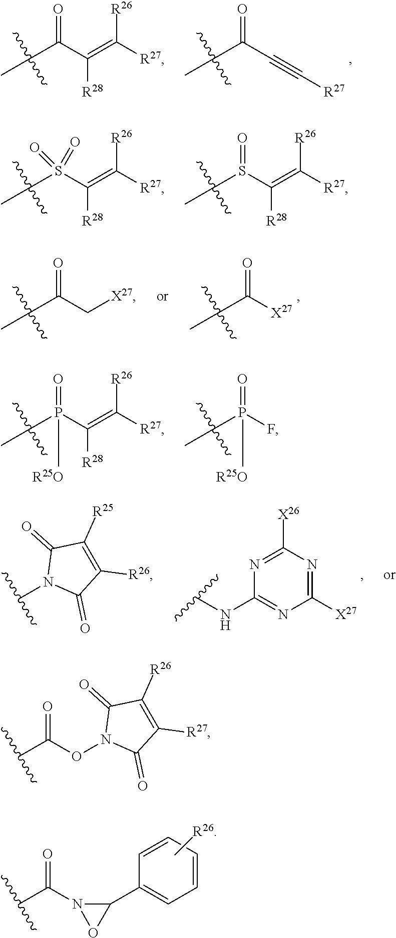

R²⁶, R²⁷, R²⁸, and X²⁷ are as described herein. X²⁶ is independently a halogen. In embodiments, X²⁶ is independently —Cl. In embodiments, X²⁶ is independently —Br. In embodiments, X²⁶ is independently —F. In embodiments, X²⁶ is independently —I.

R²⁵ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁵ is independently hydrogen, substituted or unsubstituted C₁-C₄ alkyl, or substituted or unsubstituted 2 to 4 membered heteroalkyl. In embodiments, R²⁵ is independently hydrogen. In embodiments, R²⁵ is independently substituted or unsubstituted C₁-C₄ alkyl. In embodiments, R²⁵ is independently substituted or unsubstituted 2 to 4 membered heteroalkyl. In embodiments, R²⁵ is independently unsubstituted C₁-C₄ alkyl. In embodiments, R²⁵ is independently unsubstituted 2 to 4 membered heteroalkyl. In embodiments, R²⁵ is independently substituted or unsubstituted C₁-C₆ alkyl or substituted or unsubstituted 2 to 6 membered heteroalkyl. In embodiments, R²⁵ is independently —Cl. In embodiments, R²⁵ is independently —Br. In embodiments, R²⁵ is independently —F. In embodiments, R²⁵ is independently —I. In embodiments, R²⁵ is independently —CH₃. In embodiments, R²⁵ is independently —CCl₃. In embodiments, R²⁵ is independently —CBr₃. In embodiments, R²⁵ is independently —CF₃. In embodiments, R²⁵ is independently —CI₃. In embodiments, R²⁵ is independently —CHCl₂. In embodiments, R²⁵ is independently —CHBr₂. In embodiments, R²⁵ is independently —CHF₂. In embodiments, R²⁵ is independently —CHI₂. In embodiments, R²⁵ is independently —CH₂C1. In embodiments, R²⁵ is independently —CH₂Br. In embodiments, R²⁵ is independently —CH₂F. In embodiments, R²⁵ is independently —CH₂I. In embodiments, R²⁵ is independently —CN. In embodiments, R²⁵ is independently —OCH₃. In embodiments, R²⁵ is independently —NH₂. In embodiments, R²⁵ is independently —COOH. In embodiments, R²⁵ is independently —COCH₃. In embodiments, R²⁵ is independently —CONH₂. In embodiments, R²⁵ is independently —OCCl₃. In embodiments, R²⁵ is independently —OCF₃. In embodiments, R²⁵ is independently —OCBr₃. In embodiments, R²⁵ is independently —OCI₃. In embodiments, R²⁵ is independently —OCHCl₂. In embodiments, R²⁵ is independently —OCHBr₂. In embodiments, R²⁵ is independently —OCHI₂. In embodiments, R²⁵ is independently —OCHF₂. In embodiments, R²⁵ is independently —OCH₂Cl. In embodiments, R²⁵ is independently —OCH₂Br. In embodiments, R²⁵ is independently —OCH₂I. In embodiments, R²⁵ is independently —OCH₂F. In embodiments, R²⁵ is independently unsubstituted methyl. In embodiments, R²⁵ is independently —OCH₃. In embodiments, R²⁵ is independently —OCH₂CH₃. In embodiments, R²⁵ is independently —OCH(CH₃)₂. In embodiments, R²⁵ is independently —OC(CH₃)₃. In embodiments, R²⁵ is independently —CH₃. In embodiments, R²⁵ is independently —CH₂CH₃. In embodiments, R²⁵ is independently —CH(CH₃)₂. In embodiments, R²⁵ is independently —C(CH₃)₃. In embodiments, R²⁵ is independently —C(O)CH₃. In embodiments, R²⁵ is independently —C(O)CH₂CH₃. In embodiments, R²⁵ is independently —C(O)CH(CH₃)₂. In embodiments, R²⁵ is independently —C(O)C(CH₃)₃.

In embodiments, R²⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁵ is independently substituted or unsubstituted alkyl. In embodiments, R²⁵ is independently unsubstituted alkyl. In embodiments, R²⁵ is independently unsubstituted methyl. In embodiments, R²⁵ is independently unsubstituted ethyl. In embodiments, R²⁵ is independently unsubstituted propyl. In embodiments, R²⁵ is independently substituted or unsubstituted heteroalkyl. In embodiments, R²⁵ is independently unsubstituted heteroalkyl. In embodiments, R²⁵ is independently substituted or unsubstituted cycloalkyl. In embodiments, R²⁵ is independently unsubstituted cycloalkyl. In embodiments, R²⁵ is independently substituted or unsubstituted heterocycloalkyl. In embodiments, R²⁵ is independently unsubstituted heterocycloalkyl. In embodiments, R²⁵ is independently substituted or unsubstituted aryl. In embodiments, R²⁵ is independently unsubstituted phenyl. In embodiments, R²⁵ is independently substituted or unsubstituted heteroaryl. In embodiments, R²⁵ is independently unsubstituted heteroaryl. In embodiments, R²⁵ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R²⁵ is independently substituted or unsubstituted C₁-C₆ alkyl. In embodiments, R²⁵ is independently unsubstituted C₁-C₆ alkyl. In embodiments, R²⁵ is independently unsubstituted methyl. In embodiments, R²⁵ is independently unsubstituted ethyl. In embodiments, R²⁵ is independently unsubstituted propyl. In embodiments, R²⁵ is independently substituted or unsubstituted 2 to 6 membered heteroalkyl. In embodiments, R²⁵ is independently unsubstituted 2 to 6 membered heteroalkyl. In embodiments, R²⁵ is independently substituted or unsubstituted C₃-C₆ cycloalkyl. In embodiments, R²⁵ is independently unsubstituted C₃-C₆ cycloalkyl. In embodiments, R²⁵ is independently substituted or unsubstituted 3 to 6 membered heterocycloalkyl. In embodiments, R²⁵ is independently unsubstituted 3 to 6 membered heterocycloalkyl. In embodiments, R²⁵ is independently substituted or unsubstituted C₆-C₁₀ aryl. In embodiments, R²⁵ is independently unsubstituted C₆-C₁₀ aryl. In embodiments, R²⁵ is independently substituted phenyl. In embodiments, R²⁵ is independently unsubstituted phenyl. In embodiments, R²⁵ is independently substituted or unsubstituted 5 to 10 membered heteroaryl. In embodiments, R²⁵ is independently substituted or unsubstituted 5 to 6 membered heteroaryl. In embodiments, R²⁵ is independently unsubstituted 5 to 10 membered heteroaryl. In embodiments, R²⁵ is independently unsubstituted 5 to 6 membered heteroaryl.

In embodiments, a substituted R²⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁵ is substituted with a plurality of groups selected from substituent groups, size-limited substituent groups, and lower substituent groups; each substituent group, size-limited substituent group, and/or lower substituent group may optionally be different. In embodiments, when R²⁵ is substituted, it is substituted with at least one substituent group. In embodiments, when R²⁵ is substituted, it is substituted with at least one size-limited substituent group. In embodiments, when R²⁵ is substituted, it is substituted with at least one lower substituent group.

In embodiments, R³ is independently hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

R^{3A}, R^{3B}, R^{3C}, and R^{3D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 12 membered, 5 to 10 membered, 5 to 9 membered, or 5 to 6 membered).

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{3A} and R^{3B} substituents bonded to the same nitrogen atom are joined is substituted, it is substituted with at least one substituent group. In embodiments, when the ring formed when R^{3A} and R^{3B} substituents bonded to the same nitrogen atom are joined is substituted, it is substituted with at least one size-limited substituent group. In embodiments, when the ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

X³ is independently —F, —Cl, —Br, or —I.

In embodiments, X³ is independently —F. In embodiments, X³ is independently —Cl. In embodiments, X³ is independently —Br. In embodiments, X³ is independently —I.

n3 is independently an integer from 0 to 4.

In embodiments, n3 is independently 0. In embodiments, n3 is independently 1. In embodiments, n3 is independently 2. In embodiments, n3 is independently 3. In embodiments, n3 is independently 4.

m3 and v3 are independently 1 or 2.

In embodiments, m3 is independently 1. In embodiments, m3 is independently 2. In embodiments, v3 is independently 1. In embodiments, v3 is independently 2.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3A} is independently hydrogen. In embodiments, R^{3B} is independently hydrogen. In embodiments, R^{3C} is independently hydrogen. In embodiments, R^{3D} is independently hydrogen.

In embodiments, R^{3A} is independently unsubstituted C₁-C₄ alkyl. In embodiments, R^{3B} is independently unsubstituted C₁-C₄ alkyl. In embodiments, R^{3C} is independently unsubstituted C₁-C₄ alkyl. In embodiments, R^{3D} is independently unsubstituted C₁-C₄ alkyl.

In embodiments, R³ is independently hydrogen, substituted or unsubstituted C₁-C₄ alkyl, or substituted or unsubstituted 2 to 4 membered heteroalkyl. In embodiments, R³ is independently hydrogen. In embodiments, R³ is independently substituted or unsubstituted C₁-C₄ alkyl. In embodiments, R³ is independently substituted or unsubstituted 2 to 4 membered heteroalkyl. In embodiments, R³ is independently unsubstituted C₁-C₄ alkyl. In embodiments, R³ is independently unsubstituted 2 to 4 membered heteroalkyl. In embodiments, R³ is independently substituted or unsubstituted C₁-C₆ alkyl or substituted or unsubstituted 2 to 6 membered heteroalkyl. In embodiments, R³ is independently —Cl. In embodiments, R³ is independently —Br. In embodiments, R³ is independently —F. In embodiments, R³ is independently —I. In embodiments, R³ is independently —CH₃. In embodiments, R³ is independently —CCl₃. In embodiments, R³ is independently —CBr₃. In embodiments, R³ is independently —CF₃. In embodiments, R³ is independently —Cl₃. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CH₂C1. In embodiments, R³ is independently —CH₂Br. In embodiments, R³ is independently —CH₂F. In embodiments, R³ is independently —CH₂I. In embodiments, R³ is independently —CN. In embodiments, R³ is independently —OCH₃. In embodiments, R³ is independently —NH₂. In embodiments, R³ is independently —COOH. In embodiments, R³ is independently —COCH₃. In embodiments, R³ is independently —CONH₂. In embodiments, R³ is independently —OCCl₃. In embodiments, R³ is independently —OCF₃. In embodiments, R³ is independently —OCBr₃. In embodiments, R³ is independently —OCI₃. In embodiments, R³ is independently —OCHCl₂. In embodiments, R³ is independently —OCHBr₂. In embodiments, R³ is independently —OCHI₂. In embodiments, R³ is independently —OCHF₂. In embodiments, R³ is independently —OCH₂Cl. In embodiments, R³ is independently —OCH₂Br. In embodiments, R³ is independently —OCH₂I. In embodiments, R³ is independently —OCH₂F. In embodiments, R³ is independently unsubstituted methyl. In embodiments, R³ is independently —OCH₃. In embodiments, R³ is independently —OCH₂CH₃. In embodiments, R³ is independently —OCH(CH₃)₂. In embodiments, R³ is independently —OC(CH₃)₃. In embodiments, R³ is independently —CH₃. In embodiments, R³ is independently —CH₂CH₃. In embodiments, R³ is independently —CH(CH₃)₂. In embodiments, R³ is independently —C(CH₃)₃. In embodiments, R³ is independently —C(O)CH₃. In embodiments, R³ is independently —C(O)CH₂CH₃. In embodiments, R³ is independently —C(O)CH(CH₃)₂. In embodiments, R³ is independently —C(O)C(CH₃)₃.

In embodiments, R³ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently substituted or unsubstituted alkyl. In embodiments, R³ is independently unsubstituted alkyl. In embodiments, R³ is independently unsubstituted methyl. In embodiments, R³ is independently unsubstituted ethyl. In embodiments, R³ is independently unsubstituted propyl. In embodiments, R³ is independently substituted or unsubstituted heteroalkyl. In embodiments, R³ is independently unsubstituted heteroalkyl. In embodiments, R³ is independently substituted or unsubstituted cycloalkyl. In embodiments, R³ is independently unsubstituted cycloalkyl. In embodiments, R³ is independently substituted or unsubstituted heterocycloalkyl. In embodiments, R³ is independently unsubstituted heterocycloalkyl. In embodiments, R³ is independently substituted or unsubstituted aryl. In embodiments, R³ is independently unsubstituted phenyl. In embodiments, R³ is independently substituted or unsubstituted heteroaryl. In embodiments, R³ is independently unsubstituted heteroaryl. In embodiments, R³ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³ is independently substituted or unsubstituted C₁-C₆ alkyl. In embodiments, R³ is independently unsubstituted C₁-C₆ alkyl. In embodiments, R³ is independently unsubstituted methyl. In embodiments, R³ is independently unsubstituted ethyl. In embodiments, R³ is independently unsubstituted propyl. In embodiments, R³ is independently substituted or unsubstituted 2 to 6 membered heteroalkyl. In embodiments, R³ is independently unsubstituted 2 to 6 membered heteroalkyl. In embodiments, R³ is independently substituted or unsubstituted C₃-C₆ cycloalkyl. In embodiments, R³ is independently unsubstituted C₃-C₆ cycloalkyl. In embodiments, R³ is independently substituted or unsubstituted 3 to 6 membered heterocycloalkyl. In embodiments, R³ is independently unsubstituted 3 to 6 membered heterocycloalkyl. In embodiments, R³ is independently substituted or unsubstituted C₆-C₁₀ aryl. In embodiments, R³ is independently unsubstituted C₆-C₁₀ aryl. In embodiments, R³ is independently substituted phenyl. In embodiments, R³ is independently unsubstituted phenyl. In embodiments, R³ is independently substituted or unsubstituted 5 to 10 membered heteroaryl. In embodiments, R³ is independently substituted or unsubstituted 5 to 6 membered heteroaryl. In embodiments, R³ is independently unsubstituted 5 to 10 membered heteroaryl. In embodiments, R³ is independently unsubstituted 5 to 6 membered heteroaryl.

In embodiments, R³ is -L^{3A}-L^{3B}-E3.

L^{3A} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L^{3A} is independently a bond.

In embodiments, L^{3A} is independently —S(O)₂—. In embodiments, L^{3A} is independently —NH—. In embodiments, L^{3A} is independently —O—. In embodiments, L^{3A} is independently —S—. In embodiments, L^{3A} is independently —C(O)—. In embodiments, L^{3A} is independently —NHS(O)₂—. In embodiments, L^{3A} is independently —S(O)₂NH—. In embodiments, L^{3A} is independently —C(O)NH—. In embodiments, L^{3A} is independently —NHC(O)—. In embodiments, L^{3A} is independently —NHC(O)NH—. In embodiments, L^{3A} is independently —C(O)O—. In embodiments, L^{3A} is independently —OC(O)—.

In embodiments, L^{3A} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{3A} is independently substituted or unsubstituted alkylene. In embodiments, L^{3A} is independently unsubstituted alkylene. In embodiments, L^{3A} is independently unsubstituted methylene. In embodiments, L^{3A} is independently unsubstituted ethylene. In embodiments, L^{3A} is independently unsubstituted propylene. In embodiments, L^{3A} is independently substituted or unsubstituted heteroalkylene. In embodiments, L^{3A} is independently unsubstituted heteroalkylene. In embodiments, L^{3A} is independently substituted or unsubstituted cycloalkylene. In embodiments, L^{3A} is independently unsubstituted cycloalkylene. In embodiments, L^{3A} is independently substituted or unsubstituted heterocycloalkylene. In embodiments, L^{3A} is independently unsubstituted heterocycloalkylene. In embodiments, L^{3A} is independently substituted or unsubstituted arylene. In embodiments, L^{3A} is independently unsubstituted phenylene. In embodiments, L^{3A} is independently substituted or unsubstituted heteroarylene. In embodiments, L^{3A} is independently unsubstituted heteroarylene. In embodiments, L^{3A} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{3A} is independently substituted or unsubstituted C₁-C₆ alkylene. In embodiments, L^{3A} is independently unsubstituted C₁-C₆ alkylene. In embodiments, L^{3A} is independently unsubstituted methylene. In embodiments, L^{3A} is independently unsubstituted ethylene. In embodiments, L^{3A} is independently unsubstituted propylene. In embodiments, L^{3A} is independently substituted or unsubstituted 2 to 6 membered heteroalkylene. In embodiments, L^{3A} is independently unsubstituted 2 to 6 membered heteroalkylene. In embodiments, L^{3A} is independently substituted or unsubstituted C₃-C₆ cycloalkylene. In embodiments, L^{3A} is independently unsubstituted C₃-C₆ cycloalkylene. In embodiments, L^{3A} is independently substituted or unsubstituted 3 to 6 membered heterocycloalkylene. In embodiments, L^{3A} is independently unsubstituted 3 to 6 membered heterocycloalkylene. In embodiments, L^{3A} is independently substituted or unsubstituted C₆-C₁₀ arylene. In embodiments, L^{3A} is independently unsubstituted C₆-C₁₀ arylene. In embodiments, L^{3A} is independently substituted phenylene. In embodiments, L^{3A} is independently unsubstituted phenylene. In embodiments, L^{3A} is independently substituted or unsubstituted 5 to 10 membered heteroarylene. In embodiments, L^{3A} is independently substituted or unsubstituted 5 to 6 membered heteroarylene. In embodiments, L^{3A} is independently unsubstituted 5 to 10 membered heteroarylene. In embodiments, L^{3A} is independently unsubstituted 5 to 6 membered heteroarylene.

In embodiments, a substituted L^{3A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{3A} is substituted with a plurality of groups selected from substituent groups, size-limited substituent groups, and lower substituent groups; each substituent group, size-limited substituent group, and/or lower substituent group may optionally be different. In embodiments, when L^{3A} is substituted, it is substituted with at least one substituent group. In embodiments, when L^{3A} is substituted, it is substituted with at least one size-limited substituent group. In embodiments, when L^{3A} is substituted, it is substituted with at least one lower substituent group.

L^{3B} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₂, C₆-C₁₀, or phenylene), or substituted or unsubstituted heteroarylene (e.g., 5 to 12 membered, 5 to 10 membered, 5 to 9 membered, or 5 to 6 membered).

In embodiments, L³¹ is independently a bond, —NH—, —C(O)NH—, —NHC(O)—, —NHC(O)NH—, substituted or unsubstituted heteroalkylene (e.g., 2 to 8 membered, 2 to 6 membered, 4 to 6 membered, 2 to 3 membered, or 4 to 5 membered), substituted or unsubstituted heterocycloalkylene (e.g., 3 to 8 membered, 3 to 6 membered, 4 to 6 membered, 4 to 5 membered, or 5 to 6 membered), or substituted or unsubstituted heteroarylene (e.g., 5 to 12 membered, 5 to 10 membered, 5 to 9 membered, or 5 to 6 membered).

In embodiments, L³¹ is independently a bond.

In embodiments, L^{3B} is independently —NH—. In embodiments, L^{3B} is independently —C(O)NH—. In embodiments, L^{3B} is independently —NHC(O)—. In embodiments, L^{3B} is independently —NHC(O)NH—.

In embodiments, L^{3B} is independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{3B} is independently substituted or unsubstituted alkylene. In embodiments, L^{3B} is independently unsubstituted alkylene. In embodiments, L^{3B} is independently unsubstituted methylene. In embodiments, L^{3B} is independently unsubstituted ethylene. In embodiments, L^{3B} is independently unsubstituted propylene. In embodiments, L^{3B} is independently substituted or unsubstituted heteroalkylene. In embodiments, L^{3B} is independently unsubstituted heteroalkylene. In embodiments, L^{3B} is independently substituted or unsubstituted cycloalkylene. In embodiments, L^{3B} is independently unsubstituted cycloalkylene. In embodiments, L^{3B} is independently substituted or unsubstituted heterocycloalkylene. In embodiments, L^{3B} is independently unsubstituted heterocycloalkylene. In embodiments, L^{3B} is independently substituted or unsubstituted arylene. In embodiments, L^{3B} is independently unsubstituted phenylene. In embodiments, L^{3B} is independently substituted or unsubstituted heteroarylene. In embodiments, L^{3B} is independently unsubstituted heteroarylene. In embodiments, L^{3B} is independently substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 10 membered heteroarylene. In embodiments, L^{3B} is independently substituted or unsubstituted C₁-C₆ alkylene. In embodiments, L^{3B} is independently unsubstituted C₁-C₆ alkylene. In embodiments, L^{3B} is independently unsubstituted methylene. In embodiments, L^{3B} is independently unsubstituted ethylene. In embodiments, L^{3B} is independently unsubstituted propylene. In embodiments, L^{3B} is independently substituted or unsubstituted 2 to 6 membered heteroalkylene. In embodiments, L^{3B} is independently unsubstituted 2 to 6 membered heteroalkylene. In embodiments, L^{3B} is independently substituted or unsubstituted C₃-C₆ cycloalkylene. In embodiments, L^{3B} is independently unsubstituted C₃-C₆ cycloalkylene. In embodiments, L^{3B} is independently substituted or unsubstituted 3 to 6 membered heterocycloalkylene. In embodiments, L^{3B} is independently unsubstituted 3 to 6 membered heterocycloalkylene. In embodiments, L^{3B} is independently substituted or unsubstituted C₆-C₁₀ arylene. In embodiments, L^{3B} is independently unsubstituted C₆-C₁₀ arylene. In embodiments, L^{3B} is independently substituted phenylene. In embodiments, L^{3B} is independently unsubstituted phenylene. In embodiments, L^{3B} is independently substituted or unsubstituted 5 to 10 membered heteroarylene. In embodiments, L^{3B} is independently substituted or unsubstituted 5 to 6 membered heteroarylene. In embodiments, L^{3B} is independently unsubstituted 5 to 10 membered heteroarylene. In embodiments, L^{3B} is independently unsubstituted 5 to 6 membered heteroarylene.

In embodiments, a substituted L^{3B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{3B} is substituted with a plurality of groups selected from substituent groups, size-limited substituent groups, and lower substituent groups; each substituent group, size-limited substituent group, and/or lower substituent group may optionally be different. In embodiments, when L^{3B} is substituted, it is substituted with at least one substituent group. In embodiments, when L^{3B} is substituted, it is substituted with at least one size-limited substituent group. In embodiments, when L^{3B} is substituted, it is substituted with at least one lower substituent group.

E3 is a covalent cysteine modifier moiety. In embodiments, E3 is a client protein covalent binding moiety. In embodiments, E3 is —SH, —SSR³⁶,

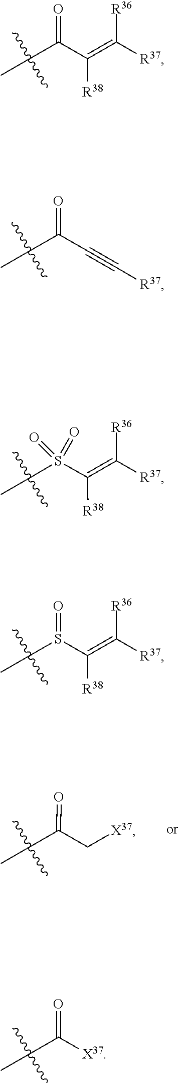

R³⁶, R³⁷, and R³⁸ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

X³⁷ is independently —F, —Cl, —Br, or —I.

In embodiments, R³⁶ is independently hydrogen, substituted or unsubstituted C₁-C₄ alkyl, or substituted or unsubstituted 2 to 4 membered heteroalkyl. In embodiments, R³⁶ is independently hydrogen. In embodiments, R³⁶ is independently substituted or unsubstituted C₁-C₄ alkyl. In embodiments, R³⁶ is independently substituted or unsubstituted 2 to 4 membered heteroalkyl. In embodiments, R³⁶ is independently unsubstituted C₁-C₄ alkyl. In embodiments, R³⁶ is independently unsubstituted 2 to 4 membered heteroalkyl. In embodiments, R³⁶ is independently substituted or unsubstituted C₁-C₆ alkyl or substituted or unsubstituted 2 to 6 membered heteroalkyl. In embodiments, R³⁶ is independently —Cl. In embodiments, R³⁶ is independently —Br. In embodiments, R³⁶ is independently —F. In embodiments, R³⁶ is independently —I. In embodiments, R³⁶ is independently —CH₃. In embodiments, R³⁶ is independently —CCl₃. In embodiments, R³⁶ is independently —CBr₃. In embodiments, R³⁶ is independently —CF₃. In embodiments, R³⁶ is independently —Cl₃. In embodiments, R³⁶ is independently —CHCl₂. In embodiments, R³⁶ is independently —CHBr₂. In embodiments, R³⁶ is independently —CHF₂. In embodiments, R³⁶ is independently —CHI₂. In embodiments, R³⁶ is independently —CH₂C1. In embodiments, R³⁶ is independently —CH₂Br. In embodiments, R³⁶ is independently —CH₂F. In embodiments, R³⁶ is independently —CH₂I. In embodiments, R³⁶ is independently —CN. In embodiments, R³⁶ is independently —OCH₃. In embodiments, R³⁶ is independently —NH₂. In embodiments, R³⁶ is independently —COOH. In embodiments, R³⁶ is independently —COCH₃. In embodiments, R³⁶ is independently —CONH₂. In embodiments, R³⁶ is independently —OCCl₃. In embodiments, R³⁶ is independently —OCF₃. In embodiments, R³⁶ is independently —OCBr₃. In embodiments, R³⁶ is independently —OCI₃. In embodiments, R³⁶ is independently —OCHCl₂. In embodiments, R³⁶ is independently —OCHBr₂. In embodiments, R³⁶ is independently —OCHI₂. In embodiments, R³⁶ is independently —OCHF₂. In embodiments, R³⁶ is independently —OCH₂Cl. In embodiments, R³⁶ is independently —OCH₂Br. In embodiments, R³⁶ is independently —OCH₂I. In embodiments, R³⁶ is independently —OCH₂F. In embodiments, R³⁶ is independently unsubstituted methyl. In embodiments, R³⁶ is independently —OCH₃. In embodiments, R³⁶ is independently —OCH₂CH₃. In embodiments, R³⁶ is independently —OCH(CH₃)₂. In embodiments, R³⁶ is independently —OC(CH₃)₃. In embodiments, R³⁶ is independently —CH₃. In embodiments, R³⁶ is independently —CH₂CH₃. In embodiments, R³⁶ is independently —CH(CH₃)₂. In embodiments, R³⁶ is independently —C(CH₃)₃. In embodiments, R³⁶ is independently —C(O)CH₃. In embodiments, R³⁶ is independently —C(O)CH₂CH₃. In embodiments, R³⁶ is independently —C(O)CH(CH₃)₂. In embodiments, R³⁶ is independently —C(O)C(CH₃)₃.

In embodiments, R³⁶ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³⁶ is independently substituted or unsubstituted alkyl. In embodiments, R³⁶ is independently unsubstituted alkyl. In embodiments, R³⁶ is independently unsubstituted methyl. In embodiments, R³⁶ is independently unsubstituted ethyl. In embodiments, R³⁶ is independently unsubstituted propyl. In embodiments, R³⁶ is independently substituted or unsubstituted heteroalkyl. In embodiments, R³⁶ is independently unsubstituted heteroalkyl. In embodiments, R³⁶ is independently substituted or unsubstituted cycloalkyl. In embodiments, R³⁶ is independently unsubstituted cycloalkyl. In embodiments, R³⁶ is independently substituted or unsubstituted heterocycloalkyl. In embodiments, R³⁶ is independently unsubstituted heterocycloalkyl. In embodiments, R³⁶ is independently substituted or unsubstituted aryl. In embodiments, R³⁶ is independently unsubstituted phenyl. In embodiments, R³⁶ is independently substituted or unsubstituted heteroaryl. In embodiments, R³⁶ is independently unsubstituted heteroaryl. In embodiments, R³⁶ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³⁶ is independently substituted or unsubstituted C₁-C₆ alkyl. In embodiments, R³⁶ is independently unsubstituted C₁-C₆ alkyl. In embodiments, R³⁶ is independently unsubstituted methyl. In embodiments, R³⁶ is independently unsubstituted ethyl. In embodiments, R³⁶ is independently unsubstituted propyl. In embodiments, R³⁶ is independently substituted or unsubstituted 2 to 6 membered heteroalkyl. In embodiments, R³⁶ is independently unsubstituted 2 to 6 membered heteroalkyl. In embodiments, R³⁶ is independently substituted or unsubstituted C₃-C₆ cycloalkyl. In embodiments, R³⁶ is independently unsubstituted C₃-C₆ cycloalkyl. In embodiments, R³⁶ is independently substituted or unsubstituted 3 to 6 membered heterocycloalkyl. In embodiments, R³⁶ is independently unsubstituted 3 to 6 membered heterocycloalkyl. In embodiments, R³⁶ is independently substituted or unsubstituted C₆-C₁₀ aryl. In embodiments, R³⁶ is independently unsubstituted C₆-C₁₀ aryl. In embodiments, R³⁶ is independently substituted phenyl. In embodiments, R³⁶ is independently unsubstituted phenyl. In embodiments, R³⁶ is independently substituted or unsubstituted 5 to 10 membered heteroaryl. In embodiments, R³⁶ is independently substituted or unsubstituted 5 to 6 membered heteroaryl. In embodiments, R³⁶ is independently unsubstituted 5 to 10 membered heteroaryl. In embodiments, R³⁶ is independently unsubstituted 5 to 6 membered heteroaryl.

In embodiments, a substituted R³⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁶ is substituted with a plurality of groups selected from substituent groups, size-limited substituent groups, and lower substituent groups; each substituent group, size-limited substituent group, and/or lower substituent group may optionally be different. In embodiments, when R³⁶ is substituted, it is substituted with at least one substituent group. In embodiments, when R³⁶ is substituted, it is substituted with at least one size-limited substituent group. In embodiments, when R³⁶ is substituted, it is substituted with at least one lower substituent group.

In embodiments, R³⁷ is independently hydrogen, substituted or unsubstituted C₁-C₄ alkyl, or substituted or unsubstituted 2 to 4 membered heteroalkyl. In embodiments, R³⁷ is independently hydrogen. In embodiments, R³⁷ is independently substituted or unsubstituted C₁-C₄ alkyl. In embodiments, R³⁷ is independently substituted or unsubstituted 2 to 4 membered heteroalkyl. In embodiments, R³⁷ is independently unsubstituted C₁-C₄ alkyl. In embodiments, R³⁷ is independently unsubstituted 2 to 4 membered heteroalkyl. In embodiments, R³⁷ is independently substituted or unsubstituted C₁-C₆ alkyl or substituted or unsubstituted 2 to 6 membered heteroalkyl. In embodiments, R³⁷ is independently —Cl. In embodiments, R³⁷ is independently —Br. In embodiments, R³⁷ is independently —F. In embodiments, R³⁷ is independently —I. In embodiments, R³⁷ is independently —CH₃. In embodiments, R³⁷ is independently —CCl₃. In embodiments, R³⁷ is independently —CBr₃. In embodiments, R³⁷ is independently —CF₃. In embodiments, R³⁷ is independently —CI₃. In embodiments, R³⁷ is independently —CHCl₂. In embodiments, R³⁷ is independently —CHBr₂. In embodiments, R³⁷ is independently —CHF₂. In embodiments, R³⁷ is independently —CHI₂. In embodiments, R³⁷ is independently —CH₂C1. In embodiments, R³⁷ is independently —CH₂Br. In embodiments, R³⁷ is independently —CH₂F. In embodiments, R³⁷ is independently —CH₂I. In embodiments, R³⁷ is independently —CN. In embodiments, R³⁷ is independently —OCH₃. In embodiments, R³⁷ is independently —NH₂. In embodiments, R³⁷ is independently —COOH. In embodiments, R³⁷ is independently —COCH₃. In embodiments, R³⁷ is independently —CONH₂. In embodiments, R³⁷ is independently —OCCl₃. In embodiments, R³⁷ is independently —OCF₃. In embodiments, R³⁷ is independently —OCBr₃. In embodiments, R³⁷ is independently —OCI₃. In embodiments, R³⁷ is independently —OCHCl₂. In embodiments, R³⁷ is independently —OCHBr₂. In embodiments, R³⁷ is independently —OCHI₂. In embodiments, R³⁷ is independently —OCHF₂. In embodiments, R³⁷ is independently —OCH₂Cl. In embodiments, R³⁷ is independently —OCH₂Br. In embodiments, R³⁷ is independently —OCH₂I. In embodiments, R³⁷ is independently —OCH₂F. In embodiments, R³⁷ is independently unsubstituted methyl. In embodiments, R³⁷ is independently —OCH₃. In embodiments, R³⁷ is independently —OCH₂CH₃. In embodiments, R³⁷ is independently —OCH(CH₃)₂. In embodiments, R³⁷ is independently —OC(CH₃)₃. In embodiments, R³⁷ is independently —CH₃. In embodiments, R³⁷ is independently —CH₂CH₃. In embodiments, R³⁷ is independently —CH(CH₃)₂. In embodiments, R³⁷ is independently —C(CH₃)₃. In embodiments, R³⁷ is independently —C(O)CH₃. In embodiments, R³⁷ is independently —C(O)CH₂CH₃. In embodiments, R³⁷ is independently —C(O)CH(CH₃)₂. In embodiments, R³⁷ is independently —C(O)C(CH₃)₃.

In embodiments, R³⁷ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³⁷ is independently substituted or unsubstituted alkyl. In embodiments, R³⁷ is independently unsubstituted alkyl. In embodiments, R³⁷ is independently unsubstituted methyl. In embodiments, R³⁷ is independently unsubstituted ethyl. In embodiments, R³⁷ is independently unsubstituted propyl. In embodiments, R³⁷ is independently substituted or unsubstituted heteroalkyl. In embodiments, R³⁷ is independently unsubstituted heteroalkyl. In embodiments, R³⁷ is independently substituted or unsubstituted cycloalkyl. In embodiments, R³⁷ is independently unsubstituted cycloalkyl. In embodiments, R³⁷ is independently substituted or unsubstituted heterocycloalkyl. In embodiments, R³⁷ is independently unsubstituted heterocycloalkyl. In embodiments, R³⁷ is independently substituted or unsubstituted aryl. In embodiments, R³⁷ is independently unsubstituted phenyl. In embodiments, R³⁷ is independently substituted or unsubstituted heteroaryl. In embodiments, R³⁷ is independently unsubstituted heteroaryl. In embodiments, R³⁷ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³⁷ is independently substituted or unsubstituted C₁-C₆ alkyl. In embodiments, R³⁷ is independently unsubstituted C₁-C₆ alkyl. In embodiments, R³⁷ is independently unsubstituted methyl. In embodiments, R³⁷ is independently unsubstituted ethyl. In embodiments, R³⁷ is independently unsubstituted propyl. In embodiments, R³⁷ is independently substituted or unsubstituted 2 to 6 membered heteroalkyl. In embodiments, R³⁷ is independently unsubstituted 2 to 6 membered heteroalkyl. In embodiments, R³⁷ is independently substituted or unsubstituted C₃-C₆ cycloalkyl. In embodiments, R³⁷ is independently unsubstituted C₃-C₆ cycloalkyl. In embodiments, R³⁷ is independently substituted or unsubstituted 3 to 6 membered heterocycloalkyl. In embodiments, R³⁷ is independently unsubstituted 3 to 6 membered heterocycloalkyl. In embodiments, R³⁷ is independently substituted or unsubstituted C₆-C₁₀ aryl. In embodiments, R³⁷ is independently unsubstituted C₆-C₁₀ aryl. In embodiments, R³⁷ is independently substituted phenyl. In embodiments, R³⁷ is independently unsubstituted phenyl. In embodiments, R³⁷ is independently substituted or unsubstituted 5 to 10 membered heteroaryl. In embodiments, R³⁷ is independently substituted or unsubstituted 5 to 6 membered heteroaryl. In embodiments, R³⁷ is independently unsubstituted 5 to 10 membered heteroaryl. In embodiments, R³⁷ is independently unsubstituted 5 to 6 membered heteroaryl.

In embodiments, a substituted R³⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁷ is substituted with a plurality of groups selected from substituent groups, size-limited substituent groups, and lower substituent groups; each substituent group, size-limited substituent group, and/or lower substituent group may optionally be different. In embodiments, when R³⁷ is substituted, it is substituted with at least one substituent group. In embodiments, when R³⁷ is substituted, it is substituted with at least one size-limited substituent group. In embodiments, when R³⁷ is substituted, it is substituted with at least one lower substituent group.

In embodiments, R³⁸ is independently hydrogen, substituted or unsubstituted C₁-C₄ alkyl, or substituted or unsubstituted 2 to 4 membered heteroalkyl. In embodiments, R³⁸ is independently hydrogen. In embodiments, R³⁸ is independently substituted or unsubstituted C₁-C₄ alkyl. In embodiments, R³⁸ is independently substituted or unsubstituted 2 to 4 membered heteroalkyl. In embodiments, R³⁸ is independently unsubstituted C₁-C₄ alkyl. In embodiments, R³⁸ is independently unsubstituted 2 to 4 membered heteroalkyl. In embodiments, R³⁸ is independently substituted or unsubstituted C₁-C₆ alkyl or substituted or unsubstituted 2 to 6 membered heteroalkyl. In embodiments, R³⁸ is independently —Cl. In embodiments, R³⁸ is independently —Br. In embodiments, R³⁸ is independently —F. In embodiments, R³⁸ is independently —I. In embodiments, R³⁸ is independently —CH₃. In embodiments, R³⁸ is independently —CCl₃. In embodiments, R³⁸ is independently —CBr₃. In embodiments, R³⁸ is independently —CF₃. In embodiments, R³⁸ is independently —CI₃. In embodiments, R³⁸ is independently —CHCl₂. In embodiments, R³⁸ is independently —CHBr₂. In embodiments, R³⁸ is independently —CHF₂. In embodiments, R³⁸ is independently —CHI₂. In embodiments, R³⁸ is independently —CH₂C1. In embodiments, R³⁸ is independently —CH₂Br. In embodiments, R³⁸ is independently —CH₂F. In embodiments, R³⁸ is independently —CH₂I. In embodiments, R³⁸ is independently —CN. In embodiments, R³⁸ is independently —OCH₃. In embodiments, R³⁸ is independently —NH₂. In embodiments, R³⁸ is independently —COOH. In embodiments, R³⁸ is independently —COCH₃. In embodiments, R³⁸ is independently —CONH₂. In embodiments, R³⁸ is independently —OCCl₃. In embodiments, R³⁸ is independently —OCF₃. In embodiments, R³⁸ is independently —OCBr₃. In embodiments, R³⁸ is independently —OCI₃. In embodiments, R³⁸ is independently —OCHCl₂. In embodiments, R³⁸ is independently —OCHBr₂. In embodiments, R³⁸ is independently —OCHI₂. In embodiments, R³⁸ is independently —OCHF₂. In embodiments, R³⁸ is independently —OCH₂Cl. In embodiments, R³⁸ is independently —OCH₂Br. In embodiments, R³⁸ is independently —OCH₂I. In embodiments, R³⁸ is independently —OCH₂F. In embodiments, R³⁸ is independently unsubstituted methyl. In embodiments, R³⁸ is independently —OCH₃. In embodiments, R³⁸ is independently —OCH₂CH₃. In embodiments, R³⁸ is independently —OCH(CH₃)₂. In embodiments, R³⁸ is independently —OC(CH₃)₃. In embodiments, R³⁸ is independently —CH₃. In embodiments, R³⁸ is independently —CH₂CH₃. In embodiments, R³⁸ is independently —CH(CH₃)₂. In embodiments, R³⁸ is independently —C(CH₃)₃. In embodiments, R³⁸ is independently —C(O)CH₃. In embodiments, R³⁸ is independently —C(O)CH₂CH₃. In embodiments, R³⁸ is independently —C(O)CH(CH₃)₂. In embodiments, R³⁸ is independently —C(O)C(CH₃)₃.

In embodiments, R³⁸ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³⁸ is independently substituted or unsubstituted alkyl. In embodiments, R³⁸ is independently unsubstituted alkyl. In embodiments, R³⁸ is independently unsubstituted methyl. In embodiments, R³⁸ is independently unsubstituted ethyl. In embodiments, R³⁸ is independently unsubstituted propyl. In embodiments, R³⁸ is independently substituted or unsubstituted heteroalkyl. In embodiments, R³⁸ is independently unsubstituted heteroalkyl. In embodiments, R³⁸ is independently substituted or unsubstituted cycloalkyl. In embodiments, R³⁸ is independently unsubstituted cycloalkyl. In embodiments, R³⁸ is independently substituted or unsubstituted heterocycloalkyl. In embodiments, R³⁸ is independently unsubstituted heterocycloalkyl. In embodiments, R³⁸ is independently substituted or unsubstituted aryl. In embodiments, R³⁸ is independently unsubstituted phenyl. In embodiments, R³⁸ is independently substituted or unsubstituted heteroaryl. In embodiments, R³⁸ is independently unsubstituted heteroaryl. In embodiments, R³⁸ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³⁸ is independently substituted or unsubstituted C₁-C₆ alkyl. In embodiments, R³⁸ is independently unsubstituted C₁-C₆ alkyl. In embodiments, R³⁸ is independently unsubstituted methyl. In embodiments, R³⁸ is independently unsubstituted ethyl. In embodiments, R³⁸ is independently unsubstituted propyl. In embodiments, R³⁸ is independently substituted or unsubstituted 2 to 6 membered heteroalkyl. In embodiments, R³⁸ is independently unsubstituted 2 to 6 membered heteroalkyl. In embodiments, R³⁸ is independently substituted or unsubstituted C₃-C₆ cycloalkyl. In embodiments, R³⁸ is independently unsubstituted C₃-C₆ cycloalkyl. In embodiments, R³⁸ is independently substituted or unsubstituted 3 to 6 membered heterocycloalkyl. In embodiments, R³⁸ is independently unsubstituted 3 to 6 membered heterocycloalkyl. In embodiments, R³⁸ is independently substituted or unsubstituted C₆-C₁₀ aryl. In embodiments, R³⁸ is independently unsubstituted C₆-C₁₀ aryl. In embodiments, R³⁸ is independently substituted phenyl. In embodiments, R³⁸ is independently unsubstituted phenyl. In embodiments, R³⁸ is independently substituted or unsubstituted 5 to 10 membered heteroaryl. In embodiments, R³⁸ is independently substituted or unsubstituted 5 to 6 membered heteroaryl. In embodiments, R³⁸ is independently unsubstituted 5 to 10 membered heteroaryl. In embodiments, R³⁸ is independently unsubstituted 5 to 6 membered heteroaryl.

In embodiments, a substituted R³⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁸ is substituted with a plurality of groups selected from substituent groups, size-limited substituent groups, and lower substituent groups; each substituent group, size-limited substituent group, and/or lower substituent group may optionally be different. In embodiments, when R³⁸ is substituted, it is substituted with at least one substituent group. In embodiments, when R³⁸ is substituted, it is substituted with at least one size-limited substituent group. In embodiments, when R³⁸ is substituted, it is substituted with at least one lower substituent group.

X³⁷ is independently —F, —Cl, —Br, or —I.

In embodiments, X³⁷ is independently —F. In embodiments, X³⁷ is independently —Cl. In embodiments, X³⁷ is independently —Br. In embodiments, X³⁷ is independently —I.

In embodiments, E3 is

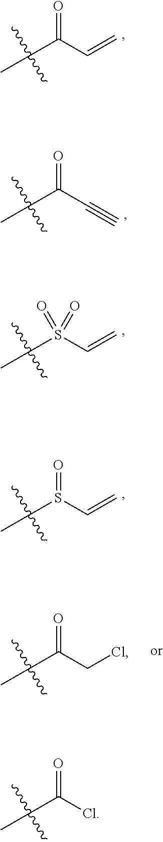

In embodiments, E3 is —SH. In embodiments, E3 is —SSR³⁶. In embodiments, E3 is

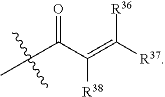

In embodiments, E3 is

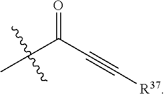

In embodiments, E3 is

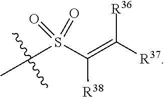

In embodiments, E3 is

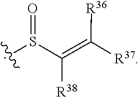

In embodiments, E3 is

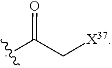

In embodiments, E3 is

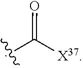

In embodiments, E3 is

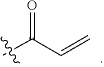

In embodiments, E3 is

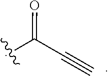

In embodiments, E3 is

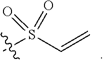

In embodiments, E3 is

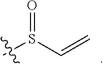

In embodiments, E3 is

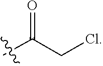

In embodiments, E3 is

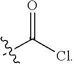

In embodiments, E3 is not —SSR³⁶. In embodiments, R³ is not —SSR³⁶. In embodiments, E3 is not —SSH. In embodiments, R³ is not —SSH. In embodiments, R³ does not include —SSR³⁶. In embodiments, R³ does not include —SSH. In embodiments, R³ does not include a disulfide. In embodiments, E3 is not —SR^{3D}. In embodiments, R³ is not —SR^{3D}In embodiments, R³ does not include —SR^{3D}. In embodiments, E3 is not —SH. In embodiments, R³ is not —SH. In embodiments, R³ does not include —SH. In embodiments, R³ does not include a thiol.

In embodiments, E3 is

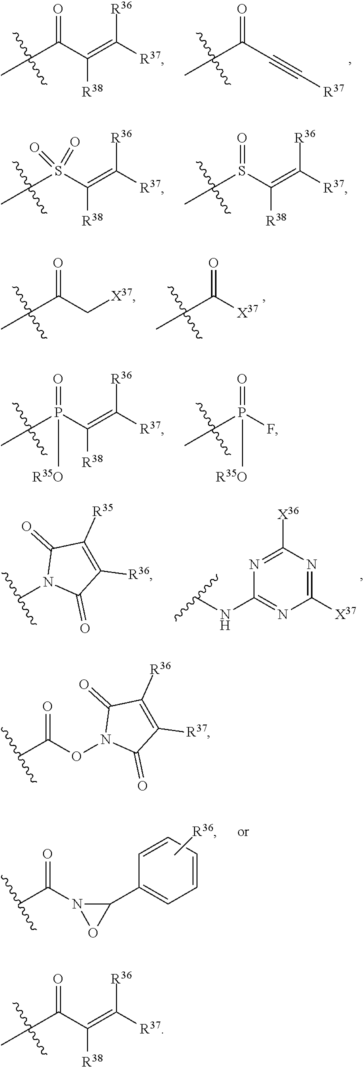

R³⁶, R³⁷, R³⁸, and X³⁷ are as described herein. X³⁶ is independently a halogen. In embodiments, X³⁶ is independently —F. In embodiments, X³⁶ is independently —Cl. In embodiments, X³⁶ is independently —Br. In embodiments, X³⁶ is independently —I.

R³⁵ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³⁵ is independently hydrogen, substituted or unsubstituted C₁-C₄ alkyl, or substituted or unsubstituted 2 to 4 membered heteroalkyl. In embodiments, R³⁵ is independently hydrogen. In embodiments, R³⁵ is independently substituted or unsubstituted C₁-C₄ alkyl. In embodiments, R³⁵ is independently substituted or unsubstituted 2 to 4 membered heteroalkyl. In embodiments, R³⁵ is independently unsubstituted C₁-C₄ alkyl. In embodiments, R³⁵ is independently unsubstituted 2 to 4 membered heteroalkyl. In embodiments, R³⁵ is independently substituted or unsubstituted C₁-C₆ alkyl or substituted or unsubstituted 2 to 6 membered heteroalkyl. In embodiments, R³⁵ is independently —Cl. In embodiments, R³⁵ is independently —Br. In embodiments, R³⁵ is independently —F. In embodiments, R³⁵ is independently —I. In embodiments, R³⁵ is independently —CH₃. In embodiments, R³⁵ is independently —CCl₃. In embodiments, R³⁵ is independently —CBr₃. In embodiments, R³⁵ is independently —CF₃. In embodiments, R³⁵ is independently —CI₃. In embodiments, R³⁵ is independently —CHCl₂. In embodiments, R³⁵ is independently —CHBr₂. In embodiments, R³⁵ is independently —CHF₂. In embodiments, R³⁵ is independently —CHI₂. In embodiments, R³⁵ is independently —CH₂C1. In embodiments, R³⁵ is independently —CH₂Br. In embodiments, R³⁵ is independently —CH₂F. In embodiments, R³⁵ is independently —CH₂I. In embodiments, R³⁵ is independently —CN. In embodiments, R³⁵ is independently —OCH₃. In embodiments, R³⁵ is independently —NH₂. In embodiments, R³⁵ is independently —COOH. In embodiments, R³⁵ is independently —COCH₃. In embodiments, R³⁵ is independently —CONH₂. In embodiments, R³⁵ is independently —OCCl₃. In embodiments, R³⁵ is independently —OCF₃. In embodiments, R³⁵ is independently —OCBr₃. In embodiments, R³⁵ is independently —OCI₃. In embodiments, R³⁵ is independently —OCHCl₂. In embodiments, R³⁵ is independently —OCHBr₂. In embodiments, R³⁵ is independently —OCHI₂. In embodiments, R³⁵ is independently —OCHF₂. In embodiments, R³⁵ is independently —OCH₂Cl. In embodiments, R³⁵ is independently —OCH₂Br. In embodiments, R³⁵ is independently —OCH₂I. In embodiments, R³⁵ is independently —OCH₂F. In embodiments, R³⁵ is independently unsubstituted methyl. In embodiments, R³⁵ is independently —OCH₃. In embodiments, R³⁵ is independently —OCH₂CH₃. In embodiments, R³⁵ is independently —OCH(CH₃)₂. In embodiments, R³⁵ is independently —OC(CH₃)₃. In embodiments, R³⁵ is independently —CH₃. In embodiments, R³⁵ is independently —CH₂CH₃. In embodiments, R³⁵ is independently —CH(CH₃)₂. In embodiments, R³⁵ is independently —C(CH₃)₃. In embodiments, R³⁵ is independently —C(O)CH₃. In embodiments, R³⁵ is independently —C(O)CH₂CH₃. In embodiments, R³⁵ is independently —C(O)CH(CH₃)₂. In embodiments, R³⁵ is independently —C(O)C(CH₃)₃.

In embodiments, R³⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³⁵ is independently substituted or unsubstituted alkyl. In embodiments, R³⁵ is independently unsubstituted alkyl. In embodiments, R³⁵ is independently unsubstituted methyl. In embodiments, R³⁵ is independently unsubstituted ethyl. In embodiments, R³⁵ is independently unsubstituted propyl. In embodiments, R³⁵ is independently substituted or unsubstituted heteroalkyl. In embodiments, R³⁵ is independently unsubstituted heteroalkyl. In embodiments, R³⁵ is independently substituted or unsubstituted cycloalkyl. In embodiments, R³⁵ is independently unsubstituted cycloalkyl. In embodiments, R³⁵ is independently substituted or unsubstituted heterocycloalkyl. In embodiments, R³⁵ is independently unsubstituted heterocycloalkyl. In embodiments, R³⁵ is independently substituted or unsubstituted aryl. In embodiments, R³⁵ is independently unsubstituted phenyl. In embodiments, R³⁵ is independently substituted or unsubstituted heteroaryl. In embodiments, R³⁵ is independently unsubstituted heteroaryl. In embodiments, R³⁵ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R³⁵ is independently substituted or unsubstituted C₁-C₆ alkyl. In embodiments, R³⁵ is independently unsubstituted C₁-C₆ alkyl. In embodiments, R³⁵ is independently unsubstituted methyl. In embodiments, R³⁵ is independently unsubstituted ethyl. In embodiments, R³⁵ is independently unsubstituted propyl. In embodiments, R³⁵ is independently substituted or unsubstituted 2 to 6 membered heteroalkyl. In embodiments, R³⁵ is independently unsubstituted 2 to 6 membered heteroalkyl. In embodiments, R³⁵ is independently substituted or unsubstituted C₃-C₆ cycloalkyl. In embodiments, R³⁵ is independently unsubstituted C₃-C₆ cycloalkyl. In embodiments, R³⁵ is independently substituted or unsubstituted 3 to 6 membered heterocycloalkyl. In embodiments, R³⁵ is independently unsubstituted 3 to 6 membered heterocycloalkyl. In embodiments, R³⁵ is independently substituted or unsubstituted C₆-C₁₀ aryl. In embodiments, R³⁵ is independently unsubstituted C₆-C₁₀ aryl. In embodiments, R³⁵ is independently substituted phenyl. In embodiments, R³⁵ is independently unsubstituted phenyl. In embodiments, R³⁵ is independently substituted or unsubstituted 5 to 10 membered heteroaryl. In embodiments, R³⁵ is independently substituted or unsubstituted 5 to 6 membered heteroaryl. In embodiments, R³⁵ is independently unsubstituted 5 to 10 membered heteroaryl. In embodiments, R³⁵ is independently unsubstituted 5 to 6 membered heteroaryl.

In embodiments, a substituted R³⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁵ is substituted with a plurality of groups selected from substituent groups, size-limited substituent groups, and lower substituent groups; each substituent group, size-limited substituent group, and/or lower substituent group may optionally be different. In embodiments, when R³⁵ is substituted, it is substituted with at least one substituent group. In embodiments, when R³⁵ is substituted, it is substituted with at least one size-limited substituent group. In embodiments, when R³⁵ is substituted, it is substituted with at least one lower substituent group.

In embodiments, R⁵ is a 14-3-3β D215 binding moiety (14-3-3beta). In embodiments, R⁵ is a 14-3-3ε D216 binding moiety (14-3-3epsilon). In embodiments, R⁵ is a 14-3-3η D218 binding moiety (14-3-3eta). In embodiments, R⁵ is a 14-3-3γ D218 binding moiety (14-3-3gamma). In embodiments, R⁵ is a 14-3-3σ D215 binding moiety (14-3-3sigma). In embodiments, R⁵ is a 14-3-3τ D213 binding moiety (14-3-3tau). In embodiments, R⁵ is a 14-3-3ζ D213 binding moiety (14-3-3zeta).

In embodiments, R⁵ is a 14-3-3 D215 covalent binding moiety. In embodiments, R⁵ is a 14-3-3 D215 non-covalent binding moiety.

In embodiments, R⁵ is a 14-3-3 D215 covalent binding moiety.

In embodiments, R⁵ is a 14-3-3β D215 covalent binding moiety (14-3-3beta). In embodiments, R⁵ is a 14-3-3ε D216 covalent binding moiety (14-3-3epsilon). In embodiments, R⁵ is a 14-3-3η D218 covalent binding moiety (14-3-3eta). In embodiments, R⁵ is a 14-3-3γ D218 covalent binding moiety (14-3-3gamma). In embodiments, R⁵ is a 14-3-3σ D215 covalent binding moiety (14-3-3sigma). In embodiments, R⁵ is a 14-3-3τ D213 covalent binding moiety (14-3-3tau). In embodiments, R⁵ is a 14-3-3ζ D213 covalent binding moiety (14-3-3zeta).

In embodiments, R⁵ is a 14-3-3 D215 non-covalent binding moiety.

In embodiments, R⁵ is a 14-3-3β D215 non-covalent binding moiety (14-3-3beta). In embodiments, R⁵ is a 14-3-3ε D216 non-covalent binding moiety (14-3-3epsilon). In embodiments, R⁵ is a 14-3-3η D218 non-covalent binding moiety (14-3-3eta). In embodiments, R⁵ is a 14-3-3γ D218 non-covalent binding moiety (14-3-3gamma). In embodiments, R⁵ is a 14-3-3σ D215 non-covalent binding moiety (14-3-3sigma). In embodiments, R⁵ is a 14-3-3τ D213 non-covalent binding moiety (14-3-3tau). In embodiments, R⁵ is a 14-3-3ζ D213 non-covalent binding moiety (14-3-3zeta).

In embodiments, R⁵ is independently hydrogen, halogen, —CX⁵₃, —CHX⁵₂, —CH₂X⁵, —OCX⁵₃, —OCH₂X⁵, —OCHX⁵₂, —CN, —SO₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered).

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 12 membered, 5 to 10 membered, 5 to 9 membered, or 5 to 6 membered).

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{5A} and R^{5B} substituents bonded to the same nitrogen atom are joined is substituted, it is substituted with at least one substituent group. In embodiments, when the ring formed when R^{5A} and R^{5B} substituents bonded to the same nitrogen atom are joined is substituted, it is substituted with at least one size-limited substituent group. In embodiments, when the ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R^{5A} is independently hydrogen. In embodiments, R^{5B} is independently hydrogen. In embodiments, R^{5C} is independently hydrogen. In embodiments, R^{5D} is independently hydrogen.

In embodiments, R^{5A} is independently unsubstituted C₁-C₄ alkyl. In embodiments, R^{5B} is independently unsubstituted C₁-C₄ alkyl. In embodiments, R^{5C} is independently unsubstituted C₁-C₄ alkyl. In embodiments, R^{5D} is independently unsubstituted C₁-C₄ alkyl.

In embodiments, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted C₆-C₁₀ aryl, or unsubstituted 5 to 10 membered heteroaryl.

X⁵ is independently —F, —Cl, —Br, or —I.

In embodiments, X⁵ is independently —F. In embodiments, X⁵ is independently —Cl. In embodiments, X⁵ is independently —Br. In embodiments, X⁵ is independently —I.

n5 is independently an integer from 0 to 4.

In embodiments, n5 is independently 0. In embodiments, n5 is independently 1. In embodiments, n5 is independently 2. In embodiments, n5 is independently 3. In embodiments, n5 is independently 4.

m5 and v5 are independently 1 or 2.

In embodiments, m5 is independently 1. In embodiments, m5 is independently 2. In embodiments, v5 is independently 1. In embodiments, v5 is independently 2.

In embodiments, R⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —C(NH)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R⁵ is independently hydrogen. In embodiments, R⁵ is independently halogen. In embodiments, R⁵ is independently —CCl₃. In embodiments, R⁵ is independently —CBr₃. In embodiments, R⁵ is independently —CF₃. In embodiments, R⁵ is independently —CI₃. In embodiments, R⁵ is independently —CH₂C1. In embodiments, R⁵ is independently —CH₂Br. In embodiments, R⁵ is independently —CH₂F. In embodiments, R⁵ is independently —CH₂I. In embodiments, R⁵ is independently —CHCl₂. In embodiments, R⁵ is independently —CHBr₂. In embodiments, R⁵ is independently —CHF₂. In embodiments, R⁵ is independently —CHI₂. In embodiments, R⁵ is independently —CN. In embodiments, R⁵ is independently —OH. In embodiments, R⁵ is independently —NH₂. In embodiments, R⁵ is independently —COOH. In embodiments, R⁵ is independently —CONH₂. In embodiments, R⁵ is independently —NO₂. In embodiments, R⁵ is independently —SH. In embodiments, R⁵ is independently —SO₃H. In embodiments, R⁵ is independently —SO₄H. In embodiments, R⁵ is independently —SO₂NH₂. In embodiments, R⁵ is independently —NHNH₂. In embodiments, R⁵ is independently —ONH₂. In embodiments, R⁵ is independently —NHC(O)NHNH₂. In embodiments, R⁵ is independently —NHC(O)NH₂. In embodiments, R⁵ is independently —NHSO₂H. In embodiments, R⁵ is independently —NHC(O)H. In embodiments, R⁵ is independently —NHC(O)OH. In embodiments, R⁵ is independently —NHC(NH)H. In embodiments, R⁵ is independently —NHC(NH)NH₂. In embodiments, R⁵ is independently —NHOH. In embodiments, R⁵ is independently —OCCl₃. In embodiments, R⁵ is independently —OCBr₃. In embodiments, R⁵ is independently —OCF₃. In embodiments, R⁵ is independently —OCI₃. In embodiments, R⁵ is independently —OCH₂Cl. In embodiments, R⁵ is independently —OCH₂Br. In embodiments, R⁵ is independently —OCH₂F. In embodiments, R⁵ is independently —OCH₂I. In embodiments, R⁵ is independently —OCHCl₂. In embodiments, R⁵ is independently —OCHBr₂. In embodiments, R⁵ is independently —OCHF₂. In embodiments, R⁵ is independently —OCHI₂. In embodiments, R⁵ is independently —N₃. In embodiments, R⁵ is independently —SF₅. In embodiments, R⁵ is independently —C(NH)NH₂. In embodiments, R⁵ is independently substituted or unsubstituted alkyl (e.g., C₁-C₈, C₁-C₆, C₁-C₄, or C₁-C₂). In embodiments, R⁵ is independently substituted alkyl (e.g., C₁-C₈, C₁-C₆, C₁-C₄, or C₁-C₂). In embodiments, R⁵ is independently unsubstituted alkyl (e.g., C₁-C₈, C₁-C₆, C₁-C₄, or C₁-C₂). In embodiments, R⁵ is independently substituted or unsubstituted heteroalkyl (e.g., 2 to 8 membered, 2 to 6 membered, 4 to 6 membered, 2 to 3 membered, or 4 to 5 membered). In embodiments, R⁵ is independently substituted heteroalkyl (e.g., 2 to 8 membered, 2 to 6 membered, 4 to 6 membered, 2 to 3 membered, or 4 to 5 membered). In embodiments, R⁵ is independently unsubstituted heteroalkyl (e.g., 2 to 8 membered, 2 to 6 membered, 4 to 6 membered, 2 to 3 membered, or 4 to 5 membered). In embodiments, R⁵ is independently substituted or unsubstituted cycloalkyl (e.g., C₃-C₈, C₃-C₆, C₄-C₆, or C₅-C₅). In embodiments, R⁵ is independently substituted cycloalkyl (e.g., C₃-C₈, C₃-C₆, C₄-C₆, or C₅-C₆). In embodiments, R⁵ is independently unsubstituted cycloalkyl (e.g., C₃-C₈, C₃-C₆, C₄-C₆, or C₅-C₆). In embodiments, R⁵ is independently substituted or unsubstituted heterocycloalkyl (e.g., 3 to 8 membered, 3 to 6 membered, 4 to 6 membered, 4 to 5 membered, or 5 to 6 membered). In embodiments, R⁵ is independently substituted heterocycloalkyl (e.g., 3 to 8 membered, 3 to 6 membered, 4 to 6 membered, 4 to 5 membered, or 5 to 6 membered). In embodiments, R⁵ is independently unsubstituted heterocycloalkyl (e.g., 3 to 8 membered, 3 to 6 membered, 4 to 6 membered, 4 to 5 membered, or 5 to 6 membered). In embodiments, R⁵ is independently substituted or unsubstituted aryl (e.g., C₆-C₁₀ or phenyl). In embodiments, R⁵ is independently substituted aryl (e.g., C₆-C₁₀ or phenyl). In embodiments, R⁵ is independently unsubstituted aryl (e.g., C₆-C₁₀ or phenyl). In embodiments, R⁵ is independently substituted or unsubstituted heteroaryl (e.g., 5 to 10 membered, 5 to 9 membered, or 5 to 6 membered). In embodiments, R⁵ is independently substituted heteroaryl (e.g., 5 to 10 membered, 5 to 9 membered, or 5 to 6 membered). In embodiments, R⁵ is independently unsubstituted heteroaryl (e.g., 5 to 10 membered, 5 to 9 membered, or 5 to 6 membered). In embodiments, R⁵ is independently unsubstituted methyl. In embodiments, R⁵ is independently —OCH₃. In embodiments, R⁵ is independently —OCH₂CH₃. In embodiments, R⁵ is independently —OCH(CH₃)₂. In embodiments, R⁵ is independently —OC(CH₃)₃. In embodiments, R⁵ is independently —CH₃. In embodiments, R⁵ is independently —CH₂CH₃. In embodiments, R⁵ is independently —CH(CH₃)₂. In embodiments, R⁵ is independently —C(CH₃)₃. In embodiments, R⁵ is independently —C(O)CH₃. In embodiments, R⁵ is independently —C(O)CH₂CH₃. In embodiments, R⁵ is independently —C(O)CH(CH₃)₂. In embodiments, R⁵ is independently —C(O)C(CH₃)₃. In embodiments, R⁵ is independently substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl. In embodiments, R⁵ is independently substituted or unsubstituted C₁-C₆ alkyl. In embodiments, R⁵ is independently unsubstituted C₁-C₆ alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently substituted or unsubstituted 2 to 6 membered heteroalkyl. In embodiments, R⁵ is independently unsubstituted 2 to 6 membered heteroalkyl. In embodiments, R⁵ is independently substituted or unsubstituted C₃-C₆ cycloalkyl. In embodiments, R⁵ is independently unsubstituted C₃-C₆ cycloalkyl. In embodiments, R⁵ is independently substituted or unsubstituted 3 to 6 membered heterocycloalkyl. In embodiments, R⁵ is independently unsubstituted 3 to 6 membered heterocycloalkyl. In embodiments, R⁵ is independently substituted or unsubstituted C₆-C₁₀ aryl. In embodiments, R⁵ is independently unsubstituted C₆-C₁₀ aryl. In embodiments, R⁵ is independently substituted phenyl. In embodiments, R⁵ is independently unsubstituted phenyl. In embodiments, R⁵ is independently substituted or unsubstituted 5 to 10 membered heteroaryl. In embodiments, R⁵ is independently substituted or unsubstituted 5 to 6 membered heteroaryl. In embodiments, R⁵ is independently unsubstituted 5 to 10 membered heteroaryl. In embodiments, R⁵ is independently unsubstituted 5 to 6 membered heteroaryl.

In embodiments, when R¹ is substituted, R¹ is substituted with one or more first substituent groups denoted by R¹¹ as explained in the definitions section above in the description of “first substituent group(s)”. In embodiments, when an R¹¹ substituent group is substituted, the R¹¹ substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R¹³ have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A}, R^{1A.2}, and R^{1A3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2A}, R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D}0.1 as explained in the definitions section above in the description of “first substituent group(s)”. In embodiments, when an R^{2D}0.1 substituent group is substituted, the R^{2D}0.1 substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}-1, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}-1, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3.3} is substituted, R^{3.3} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}-1, R^{WW.2}, and R^{WW.3} correspond to R^{5B}, R^{5B.1}, R^{5B.2}, and R^{5B.3}, respectively.

In embodiments, when R^{5A} and R^{5B} substituents that are bonded to the same nitrogen atom are joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents that are bonded to the same nitrogen atom are joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R^{11.1}, R^{11.2}, and R^{11.3}, respectively.

In embodiments, when R¹⁵ is substituted, R¹⁵ is substituted with one or more first substituent groups denoted by R^{15.1} as explained in the definitions section above in the description of “first substituent group(s)”. In embodiments, when an R^{15.1} substituent group is substituted, the R^{15.1} substituent group is substituted with one or more second substituent groups denoted by R^{15.2} as explained in the definitions section above in the description of “first substituent group(s)”. In embodiments, when an R^{15.2} substituent group is substituted, the R^{15.2} substituent group is substituted with one or more third substituent groups denoted by R^{15.3} as explained in the definitions section above in the description of “first substituent group(s)”. In the above embodiments, R¹⁵, R^{15.1}, R^{15.2}, and R^{15.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁵, R^{15.1}, R^{15.2}, and R^{15.3}, respectively.

In embodiments, when R¹⁶ is substituted, R¹⁶ is substituted with one or more first substituent groups denoted by R^{16.1} as explained in the definitions section above in the description of “first substituent group(s)”. In embodiments, when an R^{16.1} substituent group is substituted, the R^{16.1} substituent group is substituted with one or more second substituent groups denoted by R^{16.2} as explained in the definitions section above in the description of “first substituent group(s)”. In embodiments, when an R^{16.2} substituent group is substituted, the R^{16.2} substituent group is substituted with one or more third substituent groups denoted by R^{16.3} as explained in the definitions section above in the description of “first substituent group(s)”. In the above embodiments, R¹⁶, R^{16.1}, R^{16.2}, and R^{16.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁶, R^{16.1}, R^{16.2}, and R^{16.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R¹⁸ is substituted, R¹⁸ is substituted with one or more first substituent groups denoted by R^{18.1} as explained in the definitions section above in the description of “first substituent group(s)”. In embodiments, when an R^{18.1} substituent group is substituted, the R^{18.1} substituent group is substituted with one or more second substituent groups denoted by R^{18.2} as explained in the definitions section above in the description of “first substituent group(s)”. In embodiments, when an R^{18.2} substituent group is substituted, the R^{18.2} substituent group is substituted with one or more third substituent groups denoted by R^{18.3} as explained in the definitions section above in the description of “first substituent group(s)”. In the above embodiments, R¹⁸, R^{18.1}, R^{18.2}, and R^{18.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁸, R^{18.1}, R^{18.2}, and R^{18.3}, respectively.

In embodiments, when R²⁵ is substituted, R²⁵ is substituted with one or more first substituent groups denoted by R^{25.1} as explained in the definitions section above in the description of “first substituent group(s)”. In embodiments, when an R^{25.1} substituent group is substituted, the R^{25.1} substituent group is substituted with one or more second substituent groups denoted by R^{25.2} as explained in the definitions section above in the description of “first substituent group(s)”. In embodiments, when an R^{25.2} substituent group is substituted, the R^{25.2} substituent group is substituted with one or more third substituent groups denoted by R^{25.3} as explained in the definitions section above in the description of “first substituent group(s)”. In the above embodiments, R²⁵, R^{25.1}, R^{25.2}, and R^{25.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2,} and R^{WW.3} correspond to R²⁵, R^{25.1}, R^{25.2}, and R^{25.3}, respectively.

In embodiments, when R²⁶ is substituted, R²⁶ is substituted with one or more first substituent groups denoted by R^{26.1} as explained in the definitions section above in the description of “first substituent group(s)”. In embodiments, when an R^{26.1} substituent group is substituted, the R^{26.1} substituent group is substituted with one or more second substituent groups denoted by R^{26.2} as explained in the definitions section above in the description of “first substituent group(s)”. In embodiments, when an R^{26.2} substituent group is substituted, the R^{26.2} substituent group is substituted with one or more third substituent groups denoted by R^{26.3} as explained in the definitions section above in the description of “first substituent group(s)”. In the above embodiments, R²⁶, R^{26.1}, R^{26.2}, and R^{26.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁶, R^{26.1}, R^{26.2}, and R^{26.3}, respectively.

In embodiments, when R²⁷ is substituted, R²⁷ is substituted with one or more first substituent groups denoted by R^{27.1} as explained in the definitions section above in the description of “first substituent group(s)”. In embodiments, when an R^{27.1} substituent group is substituted, the R^{27.1} substituent group is substituted with one or more second substituent groups denoted by R^{27.2} as explained in the definitions section above in the description of “first substituent group(s)”. In embodiments, when an R^{27.2} substituent group is substituted, the R^{27.2} substituent group is substituted with one or more third substituent groups denoted by R^{27.3} as explained in the definitions section above in the description of “first substituent group(s)”. In the above embodiments, R²⁷, R^{27.1}, R^{27.2}, and R^{27.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁷, R^{27.1}, R^{27.2}, and R^{27.3}, respectively.

In embodiments, when R²⁸ is substituted, R²⁸ is substituted with one or more first substituent groups denoted by R^{28.1} as explained in the definitions section above in the description of “first substituent group(s)”. In embodiments, when an R^{28.1} substituent group is substituted, the R^{28.1} substituent group is substituted with one or more second substituent groups denoted by R^{28.2} as explained in the definitions section above in the description of “first substituent group(s)”. In embodiments, when an R^{28.2} substituent group is substituted, the R^{28.2} substituent group is substituted with one or more third substituent groups denoted by R^{28.3} as explained in the definitions section above in the description of “first substituent group(s)”. In the above embodiments, R²⁸, R^{28.1}, R^{28.2}, and R^{28.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁸, R^{28.1}, R^{28.2}, and R^{28.3}, respectively.

In embodiments, when R³¹ is substituted, R³¹ is substituted with one or more first substituent groups denoted by R^{31.1} as explained in the definitions section above in the description of “first substituent group(s)”. In embodiments, when an R^{31.1} substituent group is substituted, the R^{31.1} substituent group is substituted with one or more second substituent groups denoted by R^{31.2} as explained in the definitions section above in the description of “first substituent group(s)”. In embodiments, when an R^{31.2} substituent group is substituted, the R^{31.2} substituent group is substituted with one or more third substituent groups denoted by R^{31.3} as explained in the definitions section above in the description of “first substituent group(s)”. In the above embodiments, R³¹, R^{31.1}, R^{31.2}, and R^{31.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}-1, R^{WW.2}, and R^{WW.3} correspond to R³¹, R^{31.1}, R^{31.2}, and R^{31.3}, respectively.

In embodiments, when R³² is substituted, R³² is substituted with one or more first substituent groups denoted by R^{32.1} as explained in the definitions section above in the description of “first substituent group(s)”. In embodiments, when an R^{32.1} substituent group is substituted, the R^{32.1} substituent group is substituted with one or more second substituent groups denoted by R^{32.2} as explained in the definitions section above in the description of “first substituent group(s)”. In embodiments, when an R^{32.2} substituent group is substituted, the R^{32.2} substituent group is substituted with one or more third substituent groups denoted by R^{32.3} as explained in the definitions section above in the description of “first substituent group(s)”. In the above embodiments, R³², R^{32.1}, R^{32.2}, and R^{32.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³², R^{32.1}, R^{32.2}, and R^{32.3}, respectively.

In embodiments, when R³¹ and R³² substituents are joined to form a moiety that is substituted (e.g., a substituted cycloalkyl or substituted heterocycloalkyl), the moiety is substituted with one or more first substituent groups denoted by R^{31.1} as explained in the definitions section above in the description of “first substituent group(s)”. In embodiments, when an R^{31.1} substituent group is substituted, the R^{31.1} substituent group is substituted with one or more second substituent groups denoted by R^{31.2} as explained in the definitions section above in the description of “first substituent group(s)”. In embodiments, when an R^{31.2} substituent group is substituted, the R^{31.2} substituent group is substituted with one or more third substituent groups denoted by R^{31.3} as explained in the definitions section above in the description of “first substituent group(s)”. In the above embodiments, R^{31.1}, R^{31.2}, and R^{31.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{31.1}, R^{31.2}, and R^{31.3}, respectively.

In embodiments, when R³¹ and R³² substituents are optionally joined to form a moiety that is substituted (e.g., a substituted cycloalkyl or substituted heterocycloalkyl), the moiety is substituted with one or more first substituent groups denoted by R^{32.1} as explained in the definitions section above in the description of “first substituent group(s)”. In embodiments, when an R^{32.1} substituent group is substituted, the R^{32.1} substituent group is substituted with one or more second substituent groups denoted by R^{32.2} as explained in the definitions section above in the description of “first substituent group(s)”. In embodiments, when an R^{32.2} substituent group is substituted, the R^{32.2} substituent group is substituted with one or more third substituent groups denoted by R^{32.3} as explained in the definitions section above in the description of “first substituent group(s)”. In the above embodiments, R^{32.1}, R^{32.2}, and R^{32.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{32.1}, R^{32.2}, and R^{32.3}, respectively.

In embodiments, when R³⁵ is substituted, R³⁵ is substituted with one or more first substituent groups denoted by R^{35.1} as explained in the definitions section above in the description of “first substituent group(s)”. In embodiments, when an R^{35.1} substituent group is substituted, the R^{35.1} substituent group is substituted with one or more second substituent groups denoted by R^{35.2} as explained in the definitions section above in the description of “first substituent group(s)”. In embodiments, when an R^{35.2} substituent group is substituted, the R^{35.2} substituent group is substituted with one or more third substituent groups denoted by R^{35.3} as explained in the definitions section above in the description of “first substituent group(s)”. In the above embodiments, R³⁵, R^{35.1}, R^{35.2}, and R^{35.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁵, R^{35.1}, R^{35.2}, and R^{35.3}, respectively.

In embodiments, when R³⁶ is substituted, R³⁶ is substituted with one or more first substituent groups denoted by R^{36.1} as explained in the definitions section above in the description of “first substituent group(s)”. In embodiments, when an R^{36.1} substituent group is substituted, the R^{36.1} substituent group is substituted with one or more second substituent groups denoted by R^{36.2} as explained in the definitions section above in the description of “first substituent group(s)”. In embodiments, when an R^{36.2} substituent group is substituted, the R^{36.2} substituent group is substituted with one or more third substituent groups denoted by R^{36.3} as explained in the definitions section above in the description of “first substituent group(s)”. In the above embodiments, R³⁶, R^{36.1}, R^{36.2}, and R^{36.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁶, R^{36.1}, R^{36.2}, and R^{36.3}, respectively.

In embodiments, when R³⁷ is substituted, R³⁷ is substituted with one or more first substituent groups denoted by R^{37.1} as explained in the definitions section above in the description of “first substituent group(s)”. In embodiments, when an R^{37.1} substituent group is substituted, the R^{37.1} substituent group is substituted with one or more second substituent groups denoted by R^{37.2} as explained in the definitions section above in the description of “first substituent group(s)”. In embodiments, when an R^{37.2} substituent group is substituted, the R^{37.2} substituent group is substituted with one or more third substituent groups denoted by R^{37.3} as explained in the definitions section above in the description of “first substituent group(s)”. In the above embodiments, R³⁷, R^{37.1}, R^{37.2}, and R^{37.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁷, R^{37.1}, R^{37.2}, and R^{37.3}, respectively.

In embodiments, when R³⁸ is substituted, R³⁸ is substituted with one or more first substituent groups denoted by R^{38.1} as explained in the definitions section above in the description of “first substituent group(s)”. In embodiments, when an R^{38.1} substituent group is substituted, the R^{38.1} substituent group is substituted with one or more second substituent groups denoted by R^{38.2} as explained in the definitions section above in the description of “first substituent group(s)”. In embodiments, when an R^{38.2} substituent group is substituted, the R^{38.2} substituent group is substituted with one or more third substituent groups denoted by R^{38.3} as explained in the definitions section above in the description of “first substituent group(s)”. In the above embodiments, R³⁸, R^{38.1}, R^{38.2}, and R^{38.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁸, R^{38.1}, R^{38.2}, and R^{38.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L}L² substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L¹, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L², R^{L2.1}, R^{L2.2}, and R^{L2.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L3.3} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L³, R^{L3.1}, R^{L3.2}, and R^{L3.3}, respectively.

In embodiments, when L⁵ is substituted, L⁵ is substituted with one or more first substituent groups denoted by R^{L5.1} as explained in the definitions section above in the description of “first substituent group(s)”. In embodiments, when an R^{L5.1} substituent group is substituted, the R^{L5.1} substituent group is substituted with one or more second substituent groups denoted by R^{L5.2} as explained in the definitions section above in the description of “first substituent group(s)”. In embodiments, when an R^{L5.2} substituent group is substituted, the R^{L5.2} substituent group is substituted with one or more third substituent groups denoted by R^{L5.3} as explained in the definitions section above in the description of “first substituent group(s)”. In the above embodiments, L⁵, R^{L5.1}, R^{L5.2}, and R^{L5.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L⁵, R^{L5.1}, R^{L5.2}, and R^{L5.3}, respectively.

In embodiments, when L^{1A} is substituted, L^{1A} is substituted with one or more first substituent groups denoted by R^{L1A.1} as explained in the definitions section above in the description of “first substituent group(s)”. In embodiments, when an R^{L1A.1} substituent group is substituted, the R^{L1A.1} substituent group is substituted with one or more second substituent groups denoted by R^{L1A.2} as explained in the definitions section above in the description of “first substituent group(s)”. In embodiments, when an R^{L1A.2} substituent group is substituted, the R^{L1A.2} substituent group is substituted with one or more third substituent groups denoted by R^{L1A.3} as explained in the definitions section above in the description of “first substituent group(s)”. In the above embodiments, L^{1A}, R^{L1A.1}, R^{L1A.2}, and R^{L1A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1A}, R^{L1A.1}, R^{L1A.2}, and R^{L1A.3}, respectively.

In embodiments, when L^{1B} is substituted, L^{1B} is substituted with one or more first substituent groups denoted by R^{L1B.1} as explained in the definitions section above in the description of “first substituent group(s)”. In embodiments, when an R^{L1B.1} substituent group is substituted, the R^{L1B.1} substituent group is substituted with one or more second substituent groups denoted by R^{L1B.2} as explained in the definitions section above in the description of “first substituent group(s)”. In embodiments, when an R^{L1B.2} substituent group is substituted, the R^{L1B.2} substituent group is substituted with one or more third substituent groups denoted by R^{L1B.3} as explained in the definitions section above in the description of “first substituent group(s)”. In the above embodiments, L^{1B}, R^{L1B.1}, R^{L1B.2}, and R^{L1B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1B}, R^{L1B.1}, R^{L1B.2}, and R^{L1B.3}, respectively.

In embodiments, when L^{2A} is substituted, L^{2A} is substituted with one or more first substituent groups denoted by R^{L2A.1} as explained in the definitions section above in the description of “first substituent group(s)”. In embodiments, when an R^{L2A.1} substituent group is substituted, the R^{L2A.1} substituent group is substituted with one or more second substituent groups denoted by R^{L2A.2} as explained in the definitions section above in the description of “first substituent group(s)”. In embodiments, when an R^{L2A.2} substituent group is substituted, the R^{L2A.2} substituent group is substituted with one or more third substituent groups denoted by R^{L2A.3} as explained in the definitions section above in the description of “first substituent group(s)”. In the above embodiments, L^{2A}, R^{L2A.1}, R^{L2A.2}, and R^{L2A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2A}, R^{L2A.1}, R^{L2A.2}, and R^{L2A.3}, respectively.

In embodiments, when L^{2B} is substituted, L^{2B} is substituted with one or more first substituent groups denoted by R^{L2B.1} as explained in the definitions section above in the description of “first substituent group(s)”. In embodiments, when an R^{L2B.1} substituent group is substituted, the R^{L2B.1} substituent group is substituted with one or more second substituent groups denoted by R^{L2B.2} as explained in the definitions section above in the description of “first substituent group(s)”. In embodiments, when an R^{L2B.2} substituent group is substituted, the R^{L2B.2} substituent group is substituted with one or more third substituent groups denoted by R^{L2B.3} as explained in the definitions section above in the description of “first substituent group(s)”. In the above embodiments, L^{2B}, R^{L2B.1}, R^{L2B.2}, and R^{L2B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2B}, R^{L2B.1}, R^{L2B.2}, and R^{L2B.3}, respectively.

In embodiments, when L^{3A} is substituted, L^{3A} is substituted with one or more first substituent groups denoted by R^{L3A.1} as explained in the definitions section above in the description of “first substituent group(s)”. In embodiments, when an R^{L3A.1} substituent group is substituted, the R^{L3A.1} substituent group is substituted with one or more second substituent groups denoted by R^{L3A.2} as explained in the definitions section above in the description of “first substituent group(s)”. In embodiments, when an R^{L3A.2} substituent group is substituted, the R^{L3A.2} substituent group is substituted with one or more third substituent groups denoted by R^{L3A.3} as explained in the definitions section above in the description of “first substituent group(s)”. In the above embodiments, L^{3A}, R^{L3A.1}, R^{L3A.2}, and R^{L3A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3A}, R^{L3A.1}, R^{L3A.2}, and R^{L3A.3}, respectively.

In embodiments, when L^{3B} is substituted, L^{3B} is substituted with one or more first substituent groups denoted by R^{L3B.1} as explained in the definitions section above in the description of “first substituent group(s)”. In embodiments, when an R^{L3B.1} substituent group is substituted, the R^{L3B.1} substituent group is substituted with one or more second substituent groups denoted by R^{L3B.2} as explained in the definitions section above in the description of “first substituent group(s)”. In embodiments, when an R^{L3B.2} substituent group is substituted, the R^{L3B.2} substituent group is substituted with one or more third substituent groups denoted by R^{L3B.3} as explained in the definitions section above in the description of “first substituent group(s)”. In the above embodiments, L^{3B}, R^{L3B.1}, R^{L3B.2}, and R^{L3B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3B}, R^{L3B.1}, R^{L3B.2}, and R^{L3B.3}, respectively.

In embodiments, when W is substituted, W is substituted with one or more first substituent groups denoted by R^{W.1} as explained in the definitions section above in the description of “first substituent group(s)”. In embodiments, when an R^{W.1} substituent group is substituted, the R^{W.1} substituent group is substituted with one or more second substituent groups denoted by R^{W.2} as explained in the definitions section above in the description of “first substituent group(s)”. In embodiments, when an R^{W.2} substituent group is substituted, the R^{W.2} substituent group is substituted with one or more third substituent groups denoted by R^{W.3} as explained in the definitions section above in the description of “first substituent group(s)”. In the above embodiments, W, R^{W.1}, R^{W.2}, and R^{W.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are W, R^{W.1}, R^{W.2}, and R^{W.3}, respectively.

In embodiments, when W¹ is substituted, W¹ is substituted with one or more first substituent groups denoted by R^{W1.1} as explained in the definitions section above in the description of “first substituent group(s)”. In embodiments, when an R^{W1.1} substituent group is substituted, the R^{W1.1} substituent group is substituted with one or more second substituent groups denoted by R^{W1.2} as explained in the definitions section above in the description of “first substituent group(s)”. In embodiments, when an R^{W1.2} substituent group is substituted, the R^{W1.2} substituent group is substituted with one or more third substituent groups denoted by R^{W1.3} as explained in the definitions section above in the description of “first substituent group(s)”. In the above embodiments, W¹, R^{W1.1}, R^{W1.2}, and R^{W1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W¹, R^{W1.1}, R^{W1.2} and R^{W1.3}, respectively.

In embodiments, when W² is substituted, W² is substituted with one or more first substituent groups denoted by R^{W2.1} as explained in the definitions section above in the description of “first substituent group(s)”. In embodiments, when an R^{W2.1} substituent group is substituted, the R^{W2.1} substituent group is substituted with one or more second substituent groups denoted by R^{W2.2} as explained in the definitions section above in the description of “first substituent group(s)”. In embodiments, when an R^{W2.2} substituent group is substituted, the R^{W2.2} substituent group is substituted with one or more third substituent groups denoted by R^{W2.3} as explained in the definitions section above in the description of “first substituent group(s)”. In the above embodiments, W², R^{W2.1}, R^{W2.2}, and R^{W2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W², R^{W2.1}, R^{W2.2}, and R^{W2.3}, respectively.

In embodiments, when W³ is substituted, W³ is substituted with one or more first substituent groups denoted by R^{W3.1} as explained in the definitions section above in the description of “first substituent group(s)”. In embodiments, when an R^{W3.1} substituent group is substituted, the R^{W3.1} substituent group is substituted with one or more second substituent groups denoted by R^{W3.2} as explained in the definitions section above in the description of “first substituent group(s)”. In embodiments, when an R^{W3.2} substituent group is substituted, the R^{W3.2} substituent group is substituted with one or more third substituent groups denoted by R^{W3.3} as explained in the definitions section above in the description of “first substituent group(s)”. In the above embodiments, W³, R^{W3.1}, R^{W3.2}, and R^{W3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W³, R^{W3.1}, R^{W3.2}, and R^{W3.3}, respectively.

In embodiments, when W⁴ is substituted, W⁴ is substituted with one or more first substituent groups denoted by R^{W4.1} as explained in the definitions section above in the description of “first substituent group(s)”. In embodiments, when an R^{W4.1} substituent group is substituted, the R^{W4.1} substituent group is substituted with one or more second substituent groups denoted by R^{W4.2} as explained in the definitions section above in the description of “first substituent group(s)”. In embodiments, when an R^{W4.2} substituent group is substituted, the R^{W4.2} substituent group is substituted with one or more third substituent groups denoted by R^{W4.3} as explained in the definitions section above in the description of “first substituent group(s)”. In the above embodiments, W⁴, R^{W4.1}, R^{W4.2}, and R^{W4.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W⁴, R^{W4.1}, R^{W4.2}, and R^{W4.3}, respectively.

In embodiments, when W⁵ is substituted, W⁵ is substituted with one or more first substituent groups denoted by R^{W5.1} as explained in the definitions section above in the description of “first substituent group(s)”. In embodiments, when an R^{W5.1} substituent group is substituted, the R^{W5.1} substituent group is substituted with one or more second substituent groups denoted by R^{W5.2} as explained in the definitions section above in the description of “first substituent group(s)”. In embodiments, when an R^{W5.2} substituent group is substituted, the R^{W5.2} substituent group is substituted with one or more third substituent groups denoted by R^{W5.3} as explained in the definitions section above in the description of “first substituent group(s)”. In the above embodiments, W⁵, R^{W5.1}, R^{W5.2}, and R^{W5.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W⁵, R^{W5.1}, R^{W5.2}, and R^{W.3}, respectively.

In embodiments, when W⁶ is substituted, W⁶ is substituted with one or more first substituent groups denoted by R^{W6.1} as explained in the definitions section above in the description of “first substituent group(s)”. In embodiments, when an R^{W6.1} substituent group is substituted, the R^{W6.1} substituent group is substituted with one or more second substituent groups denoted by R^{W6.2} as explained in the definitions section above in the description of “first substituent group(s)”. In embodiments, when an R^{W5.2} substituent group is substituted, the R^{W6.2} substituent group is substituted with one or more third substituent groups denoted by R^{W6.3} as explained in the definitions section above in the description of “first substituent group(s)”. In the above embodiments, W⁶, R^{W6.1}, R^{W6.2}, and R^{W6.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are W⁶, R^{W6.1}, R^{W6.2}, and R^{W6.3}, respectively.

In embodiments, when E is substituted, E is substituted with one or more first substituent groups denoted by R^{E.1} as explained in the definitions section above in the description of “first substituent group(s)”. In embodiments, when an R^{E.1} substituent group is substituted, the R^{E.1} substituent group is substituted with one or more second substituent groups denoted by R^{E.2} as explained in the definitions section above in the description of “first substituent group(s)”. In embodiments, when an R^{E.2} substituent group is substituted, the R^{E.2} substituent group is substituted with one or more third substituent groups denoted by R^{E.3} as explained in the definitions section above in the description of “first substituent group(s)”. In the above embodiments, E, R^{E.1}, R^{E.2}, and R^{E.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to E, R^{E.1}, R^{E}0.2, and R^{E}0.3, respectively.

In embodiments, the compound contacts an amino acid corresponding to C38 of 14-3-3σ protein. In embodiments, the compound contacts an amino acid corresponding to N42 of 14-3-3σ protein. In embodiments, the compound contacts an amino acid corresponding to S45 of 14-3-3σ protein. In embodiments, the compound contacts an amino acid corresponding to V46 of 14-3-3σ protein. In embodiments, the compound contacts an amino acid corresponding to E115 of 14-3-3σ protein. In embodiments, the compound contacts an amino acid corresponding to F119 of 14-3-3σ protein. In embodiments, the compound contacts an amino acid corresponding to K122 of 14-3-3σ protein. In embodiments, the compound contacts an amino acid corresponding to D126 of 14-3-3σ protein. In embodiments, the compound contacts an amino acid corresponding to P167 of 14-3-3σ protein. In embodiments, the compound contacts an amino acid corresponding to 1168 of 14-3-3σ protein. In embodiments, the compound contacts an amino acid corresponding to G171 of 14-3-3σ protein. In embodiments, the compound contacts an amino acid corresponding to L172 of 14-3-3σ protein. In embodiments, the compound contacts an amino acid corresponding to L174 of 14-3-3σ protein. In embodiments, the compound contacts an amino acid corresponding to N175 of 14-3-3σ protein. In embodiments, the compound contacts an amino acid corresponding to I219 of 14-3-3σ protein. In embodiments, the compound contacts an amino acid corresponding to E39 of 14-3-3σ protein. In embodiments, the compound contacts an amino acid corresponding to R56 of 14-3-3σ protein. In embodiments, the compound contacts an amino acid corresponding to R60 of 14-3-3σ protein. In embodiments, the compound contacts an amino acid corresponding to Y130 of 14-3-3σ protein. In embodiments, the compound contacts an amino acid corresponding to E133 of 14-3-3σ protein. In embodiments, the compound contacts an amino acid corresponding to V178 of 14-3-3σ protein. In embodiments, the compound contacts an amino acid corresponding to E182 of 14-3-3σ protein. In embodiments, the compound contacts an amino acid corresponding to L222 of 14-3-3σ protein. In embodiments, the compound contacts an amino acid corresponding to D225 of 14-3-3σ protein. In embodiments, the compound contacts an amino acid corresponding to N226 of 14-3-3σ protein. In embodiments, the compound contacts an amino acid corresponding to L229.

In embodiments, the compound is a compound described herein. In embodiments, the compound, or a pharmaceutically acceptable salt thereof, is the compound. In embodiments, the compound, or a pharmaceutically acceptable salt thereof, is the pharmaceutically acceptable salt of the compound.

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is not a compound described herein (e.g., in an aspect, embodiment, figure, scheme, example, table, or claim). In embodiments, the compound is not a compound identified in a library screen as described herein (e.g., including a thiol, disulfide, or aldehyde moiety). In embodiments, the compound is not

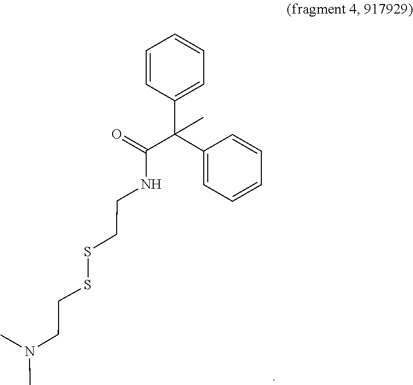 [SMILES: CN(C)CCSSCCNC(=O)C(C)(c1ccccc1)c1ccccc1]

In embodiments, the compound is not

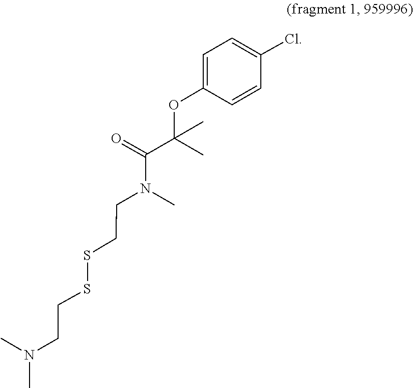 [SMILES: CN(C)CCSSCCN(C)C(=O)C(C)(C)Oc1ccc(Cl)cc1]

In embodiments, the compound is not

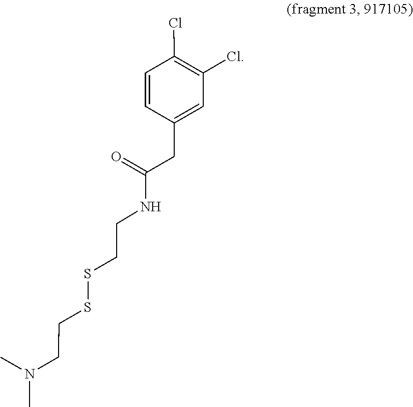 [SMILES: CN(C)CCSSCCNC(=O)Cc1ccc(Cl)c(Cl)c1]

In embodiments, the compound is not

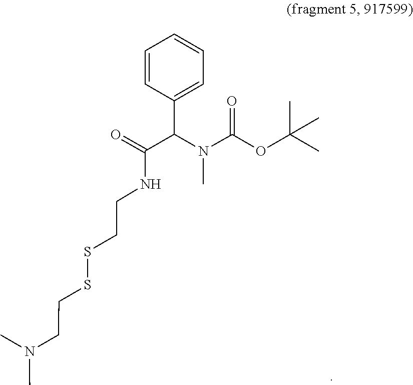 [SMILES: CN(C)CCSSCCNC(=O)C(c1ccccc1)N(C)C(=O)OC(C)(C)C]

In embodiments, the compound is not

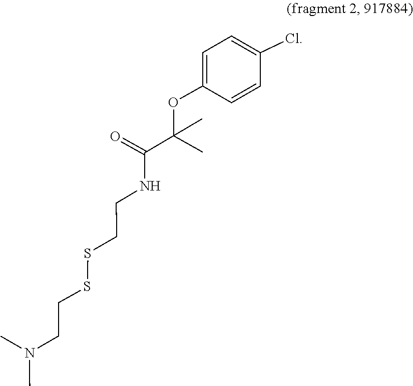 [SMILES: CN(C)CCSSCCNC(=O)C(C)(C)Oc1ccc(Cl)cc1]

In embodiments, the compound is not

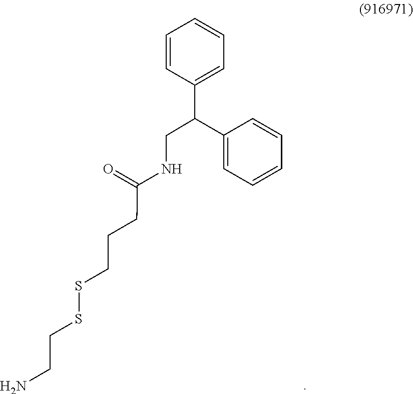 [SMILES: NCCSSCCCC(=O)NCC(c1ccccc1)c1ccccc1]

In embodiments, the compound is not

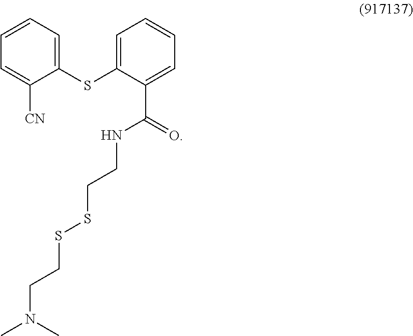 [SMILES: CN(C)CCSSCCNC(=O)c1ccccc1Sc1ccccc1C#N]

In embodiments, the compound is not

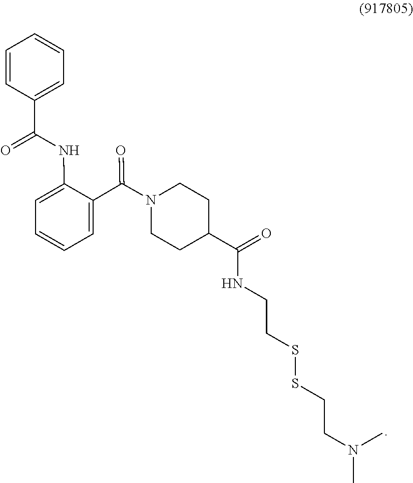 [SMILES: CN(C)CCSSCCNC(=O)C1CCN(C(=O)c2ccccc2NC(=O)c2ccccc2)CC1]

In embodiments, the compound is not

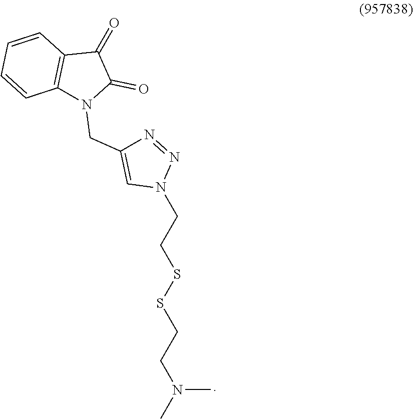 [SMILES: CN(C)CCSSCCn1cc(CN2C(=O)C(=O)c3ccccc32)nn1]

In embodiments, the compound is not

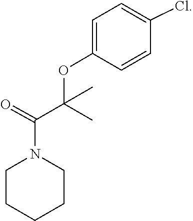 [SMILES: CC(C)(Oc1ccc(Cl)cc1)C(=O)N1CCCCC1]

In embodiments, the compound is not

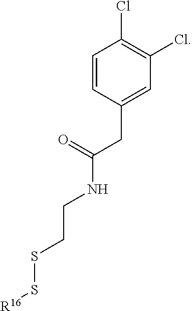

In embodiments, the compound is not

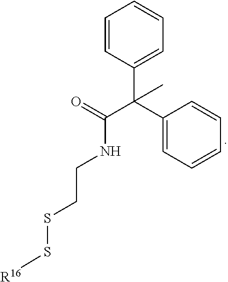

In embodiments, the compound is not

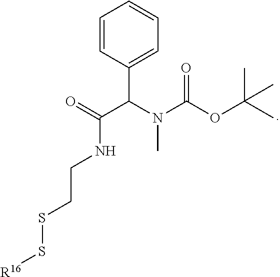

In embodiments, the compound is not

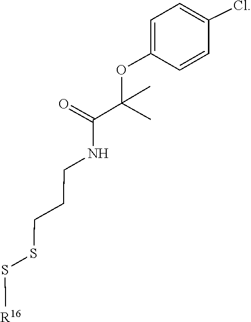

In embodiments, the compound is not

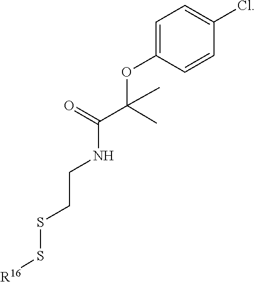

In embodiments, the compound is not

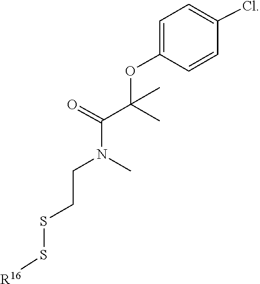

In embodiments, the compound is not

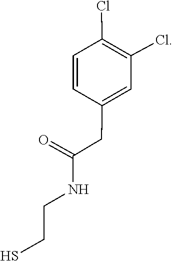 [SMILES: O=C(Cc1ccc(Cl)c(Cl)c1)NCCS]

In embodiments, the compound is not

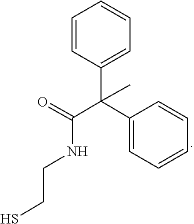 [SMILES: CC(C(=O)NCCS)(c1ccccc1)c1ccccc1]

In embodiments, the compound is not

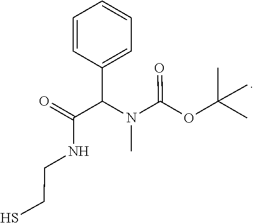 [SMILES: CN(C(=O)OC(C)(C)C)C(C(=O)NCCS)c1ccccc1]

In embodiments, the compound is not

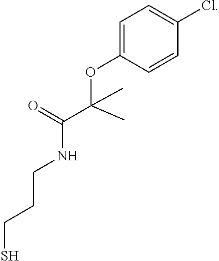 [SMILES: CC(C)(Oc1ccc(Cl)cc1)C(=O)NCCCS]

In embodiments, the compound is not

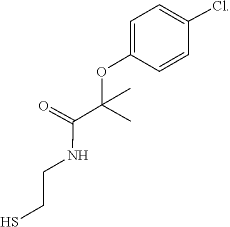 [SMILES: CC(C)(Oc1ccc(Cl)cc1)C(=O)NCCS]

In embodiments, the compound is not

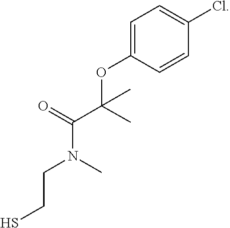 [SMILES: CN(CCS)C(=O)C(C)(C)Oc1ccc(Cl)cc1]

In embodiments, the compound does not include a moiety having the formula

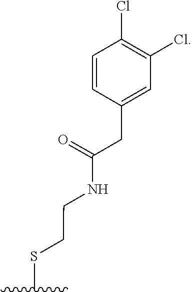 [SMILES: *SCCNC(=O)Cc1ccc(Cl)c(Cl)c1]

In embodiments, the compound does not include a moiety having the formula

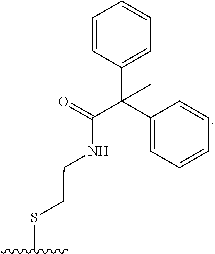 [SMILES: *SCCNC(=O)C(C)(c1ccccc1)c1ccccc1]

In embodiments, the compound does not include a moiety having the formula

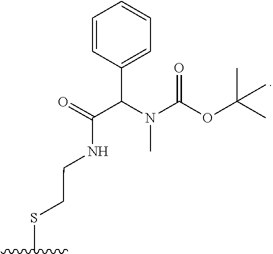 [SMILES: *SCCNC(=O)C(c1ccccc1)N(C)C(=O)OC(C)(C)C]

In embodiments, the compound does not include a moiety having the formula

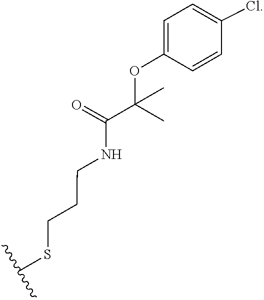 [SMILES: *SCCCNC(=O)C(C)(C)Oc1ccc(Cl)cc1]

In embodiments, the compound does not include a moiety having the formula

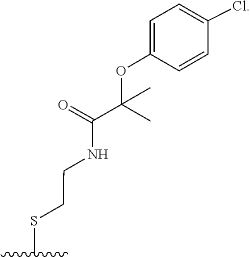 [SMILES: *SCCNC(=O)C(C)(C)Oc1ccc(Cl)cc1]

In embodiments, the compound does not include a moiety having the formula

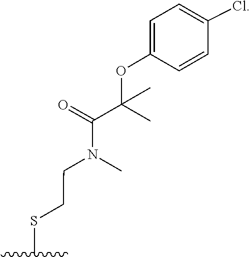 [SMILES: *SCCN(C)C(=O)C(C)(C)Oc1ccc(Cl)cc1]

### III. Stabilized Protein-Protein Complexes

In an aspect is provided an engineered stabilized protein-protein complex (such as a 14-3-3 protein-client protein complex described herein, or a conjugate-client complex described herein). The engineered stabilized protein-protein complex (e.g., the 14-3-3 protein-client protein complex or conjugate-client complex as described herein) may include: (a) a 14-3-3 protein (e.g., as described herein), (b) an exogenous protein-protein interaction (PPI) stabilizer (such as a compound described herein), e.g., having a molecular weight (Mw) of no more than 1,000 daltons (Da), and (c) a 14-3-3 client protein (e.g., as described herein). The 14-3-3 client protein may be TAZ or p65. In embodiments, the exogenous protein-protein interaction (PPI) stabilizer is a compound having the general formula R¹-L¹-W-L³-R³, wherein R¹, L¹, W, L³, and R³ are as described herein, including in embodiments. In embodiments, the exogenous protein-protein interaction (PPI) stabilizer is a compound having the general formula R²-L²-W-L³-R³, wherein R², L², W, L³, and R³ are as described herein, including in embodiments. In embodiments, the exogenous protein-protein interaction (PPI) stabilizer is a compound described herein, including in embodiments.

In embodiments of the stabilized protein-protein complex, the 14-3-3 protein and the 14-3-3 client protein define an interface. The interface may include a binding groove of the 14-3-3 protein and a 14-3-3 binding motif of the 14-3-3 client protein. The binding groove of the 14-3-3 protein may include a solvent exposed reactive amino acid side chain (e.g., as described herein) proximal to a 14-3-3 client protein binding site. The PPI stabilizer may bind with at least one amino acid residue (e.g., Cys, Lys) at the interface. The binding groove of the 14-3-3 protein may include an amino acid sequence having at least 80% sequence identity to a wild-type sequence. The 14-3-3 binding motif of the 14-3-3 client protein may include an amino acid sequence having at least 80% sequence identity to a wild-type sequence, e.g., SEQ ID NO(s) 1-2, 4, 9-14, and 43-54.

### IV. Pharmaceutical Compositions

In an aspect is provided a pharmaceutical composition including a compound described herein, or a pharmaceutically acceptable salt thereof, and a pharmaceutically acceptable excipient.

In embodiments of the pharmaceutical composition, the compound, or pharmaceutically acceptable salt thereof, is included in a therapeutically effective amount.

In embodiments of the pharmaceutical composition, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments, the second agent is an anti-cancer agent. In embodiments, the second agent is an agent useful for treating an inflammatory disease, cancer, an autoimmune disease, a neurodegenerative disease, a metabolic disease, or an infectious disease. In embodiments, the second agent is an agent useful for treating an inflammatory disease, cancer, an autoimmune disease, a neurodegenerative disease, a metabolic disease, or cystic fibrosis.

### V. Methods of Use

In an aspect is provided a method of increasing the level of a 14-3-3 protein-client protein complex in a subject, the method including administering a compound described herein to the subject.

In embodiments, the 14-3-3 client protein of the 14-3-3 protein-client protein complex is an estrogen receptor.

In embodiments, the 14-3-3 client protein of the 14-3-3 protein-client protein complex is an estrogen receptor gamma. In embodiments, the 14-3-3 client protein of the 14-3-3 protein-client protein complex is an estrogen related receptor gamma. In embodiments, the 14-3-3 client protein is ERα, ERRγ, TASK3, ExoS, MYC, Rel A, FOXO-1 or TAZ or functional replacement thereof. In embodiments, the 14-3-3 client protein is ERα, ERRγ, TASK3, ExoS, MYC, Rel A, FOXO-1 or TAZ. In embodiments, the 14-3-3 client protein is a protein described in Table 1. In embodiments, the 14-3-3 client protein is a protein described in an Example. In embodiments, the 14-3-3 client protein is a protein described herein. In embodiments, the 14-3-3 client protein is p65. In embodiments, the 14-3-3 client protein is Pin1. In embodiments, the 14-3-3 client protein is C-Raf. In embodiments, the 14-3-3 client protein is B-Raf. In embodiments, the 14-3-3 client protein is SOS. In embodiments, the 14-3-3 client protein is SOS1. In embodiments, the 14-3-3 client protein is USP8. In embodiments, the 14-3-3 client protein is ERα. In embodiments, the 14-3-3 client protein is ERRγ. In embodiments, the 14-3-3 client protein is TASK3. In embodiments, the 14-3-3 client protein is ExoS. In embodiments, the 14-3-3 client protein is MYC. In embodiments, the 14-3-3 client protein is Rel A. In embodiments, the 14-3-3 client protein is FOXO-1. In embodiments, the 14-3-3 client protein is TAZ. In embodiments, the 14-3-3 client protein is ERα, ERRγ, TASK3, ExoS, MYC, Rel A, NFκB, FOXO-1 or TAZ or functional replacement thereof.

In embodiments, the 14-3-3 client protein of the 14-3-3 protein-client protein complex is an estrogen receptor gamma. In embodiments, the 14-3-3 client protein of the 14-3-3 protein-client protein complex is an estrogen related receptor gamma. In embodiments, the 14-3-3 client protein is ERα, ERRγ, TASK3, ExoS, MYC, Rel A, FOXO-1, Cdc25A, Cdc25B, Cdc25C, Cdc2, Wee1, E2F1, ARaf, BRaf, CRaf, SLP76, BLNK, Mdm2, MdmX, PKR, RIPK2, NPM1, Pyrin, ChREBP, CIP2A, DAPK2, LDB1, MAGI1, NDE1, RND3, SSBP2, SSBP3, SSBP4, MLF1, RapGEF2, p53, Shroom3, Casp2, Cby, Tau, Ataxin1, IKBa, CFTR, TBC1D7, Gab2, USP8, SOS1, PAK6, CaMKK2, IntB2, IntAlpha4, ASKI, LRRK2, YAP, or TAZ or functional replacement thereof. In embodiments, the 14-3-3 client protein is ERα, ERRγ, TASK3, ExoS, MYC, Rel A, FOXO-1 or TAZ. In embodiments, the 14-3-3 client protein is a protein described in Table 1. In embodiments, the 14-3-3 client protein is a protein described in an Example. In embodiments, the 14-3-3 client protein is a protein described herein. In embodiments, the 14-3-3 client protein is p65. In embodiments, the 14-3-3 client protein is Pin1. In embodiments, the 14-3-3 client protein is C-Raf. In embodiments, the 14-3-3 client protein is B-Raf. In embodiments, the 14-3-3 client protein is SOS. In embodiments, the 14-3-3 client protein is SOS1. In embodiments, the 14-3-3 client protein is USP8. In embodiments, the 14-3-3 client protein is ERα. In embodiments, the 14-3-3 client protein is ERRγ. In embodiments, the 14-3-3 client protein is TASK3. In embodiments, the 14-3-3 client protein is ExoS. In embodiments, the 14-3-3 client protein is MYC. In embodiments, the 14-3-3 client protein is Rel A. In embodiments, the 14-3-3 client protein is FOXO-1. In embodiments, the 14-3-3 client protein is TAZ. In embodiments, the 14-3-3 client protein is ERα, ERRγ, TASK3, ExoS, MYC, Rel A, NFκB, FOXO-1 or TAZ or functional replacement thereof.

In embodiments, the 14-3-3 client protein of the 14-3-3 protein-client protein complex is NFκB. In embodiments, the 14-3-3 client protein of the 14-3-3 protein-client protein complex is p65. In embodiments, the 14-3-3 client protein of the 14-3-3 protein-client protein complex is Rel A.

In an aspect is provided a method of increasing the level of a 14-3-3 protein-client protein complex in a cell, the method including contacting the cell with a compound described herein.

In an aspect is provided a method of treating an inflammatory disease, cancer, an autoimmune disease, a neurodegenerative disease, a metabolic disease, or cystic fibrosis in a subject in need thereof, the method including administering to the subject in need thereof an effective amount of a compound described herein.

In an aspect is provided a method of treating an inflammatory disease, cancer, an autoimmune disease, a neurodegenerative disease, a metabolic disease, or an infectious disease in a subject in need thereof, the method including administering to the subject in need thereof an effective amount of a compound described herein.

In an aspect is provided a method of treating a 14-3-3 associated disease in a subject in need thereof, the method including administering to the subject in need thereof an effective amount of a compound described herein. In embodiments the 14-3-3 associated disease is a disease described herein.

In an aspect is provided a method of treating a cancer in a subject in need thereof, the method including administering to the subject in need thereof an effective amount of a compound described herein.

In embodiments, the cancer is breast cancer.

In embodiments, the method includes co-administering an anti-cancer agent to the subject in need. In embodiments, the method includes co-administering an agent useful for treating an inflammatory disease, cancer, an autoimmune disease, a neurodegenerative disease, a metabolic disease, or cystic fibrosis.

In an aspect is provided a method of increasing the amount of a 14-3-3 protein-client protein complex in a subject, the method including administering a compound as described herein.

In an aspect is provided a method of increasing the amount of a 14-3-3 protein-client protein complex in a cell, the method including contacting the cell with a compound as described herein.

### VI. Methods of Screening

In an aspect is provided a method of identifying a chemical compound that modulates the binding of a protein to a client protein, the method including: contacting a first candidate compound with a protein including a solvent exposed reactive amino acid side chain proximal to a client protein binding site, thereby forming a protein conjugate, wherein the first candidate compound includes a first candidate chemical moiety covalently bound to a first reactive group, wherein the first reactive group is specifically reactive with the solvent exposed reactive amino acid side chain; contacting the protein conjugate with the client protein thereby forming a conjugate-client complex; and detecting a change in stability of the conjugate-client complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates the binding of a protein to a client protein, the method including: contacting a first candidate compound with a protein including a solvent exposed reactive amino acid side chain proximal to a client protein binding site, thereby forming a protein conjugate, wherein the first candidate compound includes a first candidate chemical moiety covalently bound to a first reactive group, wherein the first reactive group is specifically reactive with the solvent exposed reactive amino acid side chain, which is not a cysteine side chain; contacting the protein conjugate with the client protein thereby forming a conjugate-client complex; and detecting a change in stability of the conjugate-client complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In embodiments, the method identifies a chemical compound that stabilizes the binding of a protein to a client protein including detecting an increase in stability of the conjugate-client complex relative to the stability of a protein-client complex.

In an aspect is provided a method of identifying a chemical compound that modulates binding of a protein to a client protein, the method including: contacting a client protein with a protein including a solvent exposed reactive amino acid side chain proximal to a client protein binding site, thereby forming a protein-client complex; contacting the protein-client complex with a first candidate compound thereby forming a conjugate-client complex, wherein the first candidate compound includes a first candidate chemical moiety covalently bound to a first reactive group, wherein the first reactive group is specifically reactive with the solvent exposed reactive amino acid side chain, and wherein the first candidate compound covalently attaches to the solvent exposed reactive amino acid side chain to form the conjugate-client complex; and detecting a change in stability of the conjugate-client complex relative to the stability of the protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates binding of a protein to a client protein, the method including: contacting a client protein with a protein including a solvent exposed reactive amino acid side chain proximal to a client protein binding site, thereby forming a protein-client complex; contacting the protein-client complex with a first candidate compound thereby forming a conjugate-client complex, wherein the first candidate compound includes a first candidate chemical moiety covalently bound to a first reactive group, wherein the first reactive group is specifically reactive with the solvent exposed reactive amino acid side chain, which is not a cysteine side chain, and wherein the first candidate compound covalently attaches to the solvent exposed reactive amino acid side chain to form the conjugate-client complex; and detecting a change in stability of the conjugate-client complex relative to the stability of the protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In embodiments, the method identifies a chemical compound that stabilizes the binding of a protein to a client protein including detecting an increase in stability of the conjugate-client complex relative to the stability of a protein-client complex.

In an aspect is provided a method of identifying a chemical compound that modulates binding of a protein to a client protein, the method including: contacting a first candidate compound with a client protein including a solvent exposed reactive amino acid side chain, thereby forming a client protein conjugate, wherein the first candidate compound includes a first candidate chemical moiety covalently bound to a first reactive group, wherein the first reactive group is specifically reactive with the solvent exposed reactive amino acid side chain; contacting the client protein conjugate with a protein thereby forming a conjugate-protein complex; and detecting a change in stability of the conjugate-protein complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that modulates binding of the protein to the client protein.

In an aspect is provided a method of identifying a chemical compound that modulates binding of a protein to a client protein, the method including: contacting a protein with a client protein including a solvent exposed reactive amino acid side chain thereby forming a protein-client complex; contacting the protein-client complex with a first candidate compound thereby forming a conjugate-protein complex, wherein the first candidate compound includes a first candidate chemical moiety covalently bound to a first reactive group, wherein the first reactive group is specifically reactive with the solvent exposed reactive amino acid side chain, and wherein the first candidate compound covalently attaches to the solvent exposed reactive amino acid side chain to form the conjugate-protein complex; and detecting a change in stability of the conjugate-protein complex relative to the stability of the protein-client complex, wherein the protein-client complex includes the protein and the client protein in the absence of the first candidate compound covalently bound to the solvent exposed reactive amino acid side chain, thereby identifying the first candidate compound as the first chemical compound that modulates binding of the protein to the client protein.

In embodiments, the method identifies a chemical compound that stabilizes the binding of a protein to a client protein including detecting an increase in stability of the conjugate-protein complex relative to the stability of a protein-client complex.

In one aspect, provided herein is a method of identifying a chemical compound that stabilizes binding of a protein (e.g., 14-3-3 protein) to a client protein. The method includes: (a) contacting a first candidate compound with a protein including a solvent exposed reactive amino acid side chain proximal to a client protein binding site, thereby forming a protein conjugate, wherein said first candidate compound includes a first candidate chemical moiety covalently bound to a first reactive group, wherein said first reactive group is specifically reactive with said solvent exposed reactive amino acid side chain; (b) contacting said protein conjugate with said client protein thereby forming a conjugate-client complex; and (c) detecting an increased stability of said conjugate-client complex relative to the stability of a protein-client complex, wherein said protein-client complex includes said client protein and said protein in the absence of said first candidate chemical compound covalently bound to said first reactive group, thereby identifying said first candidate compound that stabilizes binding of said protein to said client protein. In embodiments, the solvent exposed reactive amino acid side chain is not the side chain of the amino acid corresponding to C38 of 14-3-3 protein. In embodiments, the solvent exposed reactive amino acid side chain is not the side chain of the amino acid corresponding to C38 of 14-3-3σ protein. In embodiments, the first reactive group is not —SH. In embodiments, the first reactive group is not a substituted or unsubstituted disulfide moiety. In embodiments, the solvent exposed reactive amino acid side chain is not the side chain of the amino acid corresponding to C38 of 14-3-3 protein and the first reactive group is not —SH. In embodiments, the solvent exposed reactive amino acid side chain is not the side chain of the amino acid corresponding to C38 of 14-3-3σ protein and the first reactive group is not —SH. In embodiments, the solvent exposed reactive amino acid side chain is not the side chain of the amino acid corresponding to C38 of 14-3-3 protein and the first reactive group is not a substituted or unsubstituted disulfide moiety. In embodiments, the solvent exposed reactive amino acid side chain is not the side chain of the amino acid corresponding to C38 of 14-3-3σ protein and the first reactive group is not a substituted or unsubstituted disulfide moiety.

In embodiments, the solvent exposed reactive amino acid side chain is the side chain of the amino acid corresponding to C38 of 14-3-3 protein. In embodiments, the solvent exposed reactive amino acid side chain is the side chain of the amino acid corresponding to C38 of 14-3-3σ protein. In embodiments, the first reactive group is —SH. In embodiments, the first reactive group is a substituted or unsubstituted disulfide moiety. In embodiments, the solvent exposed reactive amino acid side chain is the side chain of the amino acid corresponding to C38 of 14-3-3 protein and the first reactive group is —SH. In embodiments, the solvent exposed reactive amino acid side chain is the side chain of the amino acid corresponding to C38 of 14-3-3σ protein and the first reactive group is —SH. In embodiments, the solvent exposed reactive amino acid side chain is the side chain of the amino acid corresponding to C38 of 14-3-3 protein and the first reactive group is a substituted or unsubstituted disulfide moiety. In embodiments, the solvent exposed reactive amino acid side chain is the side chain of the amino acid corresponding to C38 of 14-3-3σ protein and the first reactive group is a substituted or unsubstituted disulfide moiety. In embodiments, the solvent exposed reactive amino acid side chain is the side chain of the amino acid corresponding to N40 of 14-3-3β protein. In embodiments, the solvent exposed reactive amino acid side chain is the side chain of the amino acid corresponding to V39 of 14-3-3ε protein. In embodiments, the solvent exposed reactive amino acid side chain is the side chain of the amino acid corresponding to N39 of 14-3-3η protein. In embodiments, the solvent exposed reactive amino acid side chain is the side chain of the amino acid corresponding to N39 of 14-3-3γ protein. In embodiments, the solvent exposed reactive amino acid side chain is the side chain of the amino acid corresponding to N38 of 14-3-3τ protein. In embodiments, the solvent exposed reactive amino acid side chain is the side chain of the amino acid corresponding to N38 of 14-3-3 protein. In embodiments, the first candidate chemical compound has the formula R²-L²-W-L³-R³, wherein R², L², and W are as described herein, including in embodiments; L³ is a bond; and R³ is hydrogen.

In embodiments, the solvent exposed reactive amino acid side chain is the side chain of the amino acid corresponding to K120 of 14-3-3 protein. In embodiments, the solvent exposed reactive amino acid side chain is the side chain of the amino acid corresponding to K120 of 14-3-3τ protein. In embodiments, the solvent exposed reactive amino acid side chain is the side chain of the amino acid corresponding to K122 of 14-3-3β protein. In embodiments, the solvent exposed reactive amino acid side chain is the side chain of the amino acid corresponding to K123 of 14-3-3ε protein. In embodiments, the solvent exposed reactive amino acid side chain is the side chain of the amino acid corresponding to K125 of 14-3-3η protein. In embodiments, the solvent exposed reactive amino acid side chain is the side chain of the amino acid corresponding to K125 of 14-3-3γ protein. In embodiments, the solvent exposed reactive amino acid side chain is the side chain of the amino acid corresponding to K122 of 14-3-3σ protein. In embodiments, the solvent exposed reactive amino acid side chain is the side chain of the amino acid corresponding to K120 of 14-3-3ζ protein. In embodiments, the first candidate chemical compound has the formula R¹-L¹-W-L³-R³, wherein R¹, L¹, and W are as described herein, including in embodiments; L³ is a bond; and R³ is hydrogen.

In embodiments, the solvent exposed reactive amino acid side chain is the side chain of the amino acid corresponding to D215 of 14-3-3 protein. In embodiments, the solvent exposed reactive amino acid side chain is the side chain of the amino acid corresponding to D215 of 14-3-3σ protein. In embodiments, the solvent exposed reactive amino acid side chain is the side chain of the amino acid corresponding to D215 of 14-3-3β protein. In embodiments, the solvent exposed reactive amino acid side chain is the side chain of the amino acid corresponding to D215 of 14-3-3ε protein. In embodiments, the solvent exposed reactive amino acid side chain is the side chain of the amino acid corresponding to D218 of 14-3-3η protein. In embodiments, the solvent exposed reactive amino acid side chain is the side chain of the amino acid corresponding to D218 of 14-3-3η protein. In embodiments, the solvent exposed reactive amino acid side chain is the side chain of the amino acid corresponding to D213 of 14-3-3τ protein. In embodiments, the solvent exposed reactive amino acid side chain is the side chain of the amino acid corresponding to D213 of 14-3-3ζ protein.

In one aspect, provided herein is a method of identifying a chemical compound that stabilizes binding of a protein to a client protein. The method includes: (a) contacting a client protein with a protein including a solvent exposed reactive amino acid side chain proximal to a client protein binding site, thereby forming a protein-client complex; (b) contacting said protein-client complex with a first candidate compound thereby forming a conjugate-client complex, wherein said first candidate compound includes a first candidate chemical moiety covalently bound to a first reactive group, wherein said first reactive group is specifically reactive with said solvent exposed reactive amino acid side chain, and wherein said first candidate compound covalently attaches to said solvent exposed reactive amino acid side chain to form said conjugate-client complex; and (c) detecting an increased stability of said conjugate-client complex relative to the stability of said protein-client complex, wherein said protein-client complex includes said client protein and said protein in the absence of said first candidate chemical compound covalently bound to said first reactive group, thereby identifying said first candidate compound that stabilizes binding of said protein to said client protein.

In one aspect, provided herein is a method of identifying a chemical compound that stabilizes binding of a protein to a client protein. The method includes: (a) contacting a first candidate compound with a client protein including a solvent exposed reactive amino acid side chain, thereby forming a client protein conjugate, wherein said first candidate compound includes a first candidate chemical moiety covalently bound to a first reactive group, wherein said first reactive group is specifically reactive with said solvent exposed reactive amino acid side chain; (b) contacting said client protein conjugate with a protein thereby forming a conjugate-protein complex; and (c) detecting an increased stability of said conjugate-protein complex relative to the stability of a protein-client complex, wherein said protein-client complex includes said client protein and said protein in the absence of said first candidate chemical compound covalently bound to said first reactive group, thereby identifying said first candidate compound that stabilizes binding of said protein to said client protein.

In one aspect, provided herein is a method of identifying a chemical compound that stabilizes binding of a protein to a client protein. The method includes: (a) contacting a protein with a client protein including a solvent exposed reactive amino acid side chain thereby forming a protein-client complex; (b) contacting said protein-client complex with a first candidate compound thereby forming a conjugate-protein complex, wherein said first candidate compound includes a first candidate chemical moiety covalently bound to a first reactive group, wherein said first reactive group is specifically reactive with said solvent exposed reactive amino acid side chain, and wherein said first candidate compound covalently attaches to said solvent exposed reactive amino acid side chain to form said conjugate-protein complex; and (c) detecting an increased stability of said conjugate-protein complex relative to the stability of said protein-client complex, wherein said protein-client complex includes said protein and said client protein in the absence of said first candidate chemical compound covalently bound to said first reactive group, thereby identifying said first candidate compound that stabilizes binding of said protein to said client protein.

In embodiments, a chemical compound stabilizes binding of a protein to a client protein. In embodiments, a candidate chemical compound includes a candidate chemical moiety covalently bound to a reactive group. In embodiments, said candidate chemical compound is a disulfide compound. In embodiments, said candidate chemical compound is an amine compound. In embodiments, said candidate chemical compound is a carboxylic acid compound. In embodiments, said candidate chemical compound is a ketone compound. In embodiments, said candidate chemical compound is an aldehyde compound. In embodiments, said candidate chemical compound is an acrylamide compound. In embodiments, said candidate chemical compound is a vinyl sulfonamide compound. In embodiments, said candidate chemical compound is an acrylyl ester compound. In embodiments, said candidate chemical compound is identified from a library of compounds. In embodiments, said candidate chemical compound is identified from a library of disulfide compounds.

In embodiments, a first candidate compound is contacted with a protein, having a solvent exposed reactive amino acid side chain. In embodiments, said protein includes a solvent exposed reactive amino acid side chain proximal to a client protein binding site. In embodiments, a first candidate compound includes a first candidate chemical moiety covalently bound to a reactive group. In embodiments, said reactive group is specifically reactive with the solvent exposed reactive amino acid side chain of the protein.

In embodiments, the solvent exposed reactive amino acid is a natural amino acid. In embodiments, the solvent exposed reactive amino acid is a non-natural amino acid. In embodiments, the solvent exposed reactive amino acid side chain is:

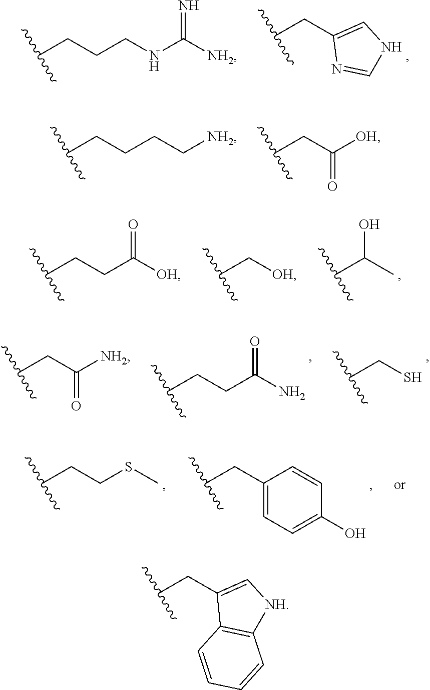

In embodiments, the solvent exposed reactive amino acid side chain is:

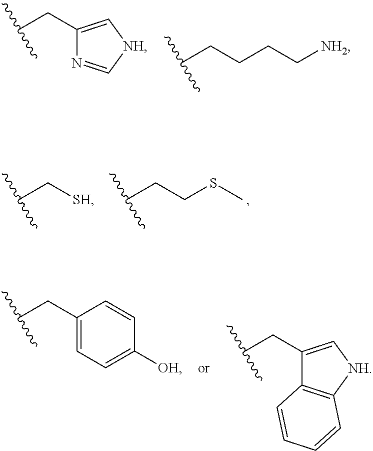

In embodiments, the solvent exposed reactive amino acid side chain is:

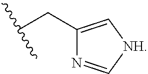 [SMILES: *Cc1c[nH]cn1]

In embodiments, the solvent exposed reactive amino acid side chain is:

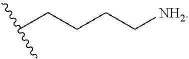 [SMILES: *CCCCN]

In embodiments, the solvent exposed reactive amino acid side chain is:

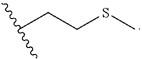 [SMILES: *CCSC]

In embodiments, the solvent exposed reactive amino acid side chain is:

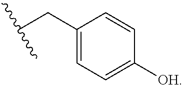 [SMILES: *Cc1ccc(O)cc1]

In embodiments, the solvent exposed reactive amino acid side chain is:

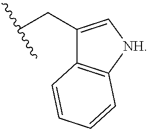 [SMILES: *Cc1c[nH]c2ccccc12]

In embodiments, the solvent exposed reactive amino acid side chain is:

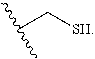 [SMILES: *CS]

In embodiments, the solvent exposed reactive amino acid side chain is not

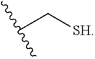 [SMILES: *CS]

In embodiments, the solvent exposed reactive amino acid side chain is the side chain of a cysteine, methionine, tryptophan, tyrosine, lysine or histidine. In embodiments, the solvent exposed reactive amino acid side chain is the side chain of a cysteine. In embodiments, the solvent exposed reactive amino acid side chain is thiol.

In embodiments, the solvent exposed reactive amino acid side chain is the side chain of a methionine, tryptophan, tyrosine, lysine or histidine. In embodiments, the solvent exposed reactive amino acid side chain is not the side chain of a cysteine. In embodiments, the solvent exposed reactive amino acid side chain is not thiol.

In embodiments, the solvent exposed reactive amino acid side chain is proximal to a client protein binding site.

In embodiments, a candidate compound includes a candidate chemical moiety covalently bound to a reactive group. In embodiments, the candidate compound is a disulfide. In embodiments, a disulfide compound is an organic compound with a disulfide moiety (—S—S—). In embodiments, the organic compound is less than 2000 Da.

In embodiments, the candidate compound is not a disulfide. In embodiments, a candidate compound is an organic compound including a disulfide linker (—S—S—). In embodiments, a candidate compound is an organic compound that does not include a disulfide linker (—S—S—).

In embodiments, the first candidate compound contacted with a protein including a solvent exposed reactive amino acid side chain proximal to a client protein binding site, forms a protein conjugate. The “protein conjugate” includes a first candidate chemical moiety covalently bound to a solvent exposed reactive amino acid side chain of the protein, proximal to a client protein binding site. In embodiments, the solvent exposed amino acid is cysteine and the candidate compound is a disulfide compound. In embodiments, the “protein conjugate” is a product of a disulfide interchange reaction, where the first candidate chemical moiety is covalently bound to the protein via a disulfide bond. In embodiments, the first candidate chemical moiety is tethered to the protein via cysteine proximal to a client protein binding site.

In embodiments, the solvent exposed amino acid is not a cysteine and the candidate compound is not a disulfide compound. In embodiments, the “protein conjugate” is not a product of a disulfide interchange reaction and the first candidate chemical moiety is not covalently bound to the protein via a disulfide bond. In embodiments, the first candidate chemical moiety is not tethered to the protein via cysteine proximal to a client protein binding site.

In embodiments, solvent exposed reactive amino acid of the protein, proximal to a client protein binding site, is a lysine. In embodiments, the candidate compound includes a reactive group. In embodiments, the reactive group is, or includes, a carboxylic acid, amide, ketone, or aldehyde. In embodiments, the amino acid is a lysine and the candidate compound is a carboxylic acid. In embodiments, the amino acid is a lysine and the candidate compound is a ketone. In embodiments, the amino acid is a lysine and the candidate compound is an aldehyde. In embodiments, the amino acid is a lysine and the candidate compound is an amide. In embodiments, the amino acid is a cysteine and the compound is a sulfide, acyl halide, or carbamoyl halide. In embodiments, the amino acid is a cysteine and the compound is a sulfide. In embodiments, the amino acid is a cysteine and the compound is an acyl halide. In embodiments, the amino acid is a cysteine and the compound is a carbamoyl halide. In embodiments, the amino acid is a histidine and the compound is a carboxylic acid, amide, ketone, or aldehyde. In embodiments, the amino acid is a histidine and the compound is a carboxylic acid. In embodiments, the amino acid is a histidine and the compound is an amide. In embodiments, the amino acid is a histidine and the compound is a ketone. In embodiments, the amino acid is a histidine and the compound is an aldehyde. In embodiments, the amino acid is a methionine and the compound is a thiol. In embodiments, the amino acid is a tyrosine and the compound is a carboxylic acid, ketone, or aldehyde. In embodiments, the amino acid is a tyrosine and the compound is a carboxylic acid. In embodiments, the amino acid is a tyrosine and the compound is a ketone. In embodiments, the amino acid is a tyrosine and the compound is an aldehyde. In embodiments, the amino acid is a tryptophan and the compound is a carboxylic acid, ketone, or aldehyde. In embodiments, the amino acid is a tryptophan and the compound is a carboxylic acid. In embodiments, the amino acid is a tryptophan and the compound is a ketone. In embodiments, the amino acid is a tryptophan and the compound is an aldehyde.

In embodiments, the amino acid is not a cysteine and the compound is not a sulfide, acyl halide, or carbamoyl halide. In embodiments, the amino acid is not a cysteine and the compound is not a sulfide. In embodiments, the amino acid is not a cysteine and the compound is not an acyl halide. In embodiments, the amino acid is not a cysteine and the compound is not a carbamoyl halide.

In embodiments, the reactive group is a bioconjugate reactive moiety as described above.

In embodiments, the reactive group is a covalent cysteine modifier, covalent lysine modifier, covalent serine modifier, or covalent methionine modifier. In embodiments, the reactive group is a covalent cysteine modifier. In embodiments, the reactive group is a covalent lysine modifier. In embodiments, the reactive group is a covalent serine modifier. In embodiments, the reactive group is a covalent methionine modifier. In embodiments, the reactive group is a covalent methionine modifier described in Lin S, Yang X, Jia S, et al. (Redox-based reagents for chemoselective methionine bioconjugation. Science (New York, N.Y.). 2017; 355(6325):597-602. doi:10.1126/science.aal3316), which is incorporated herein by reference in its entirety for all purposes.

In embodiments, the reactive group is a covalent lysine modifier, covalent serine modifier, or covalent methionine modifier. In embodiments, the reactive group is not a covalent cysteine modifier.

In embodiments, the reactive group is

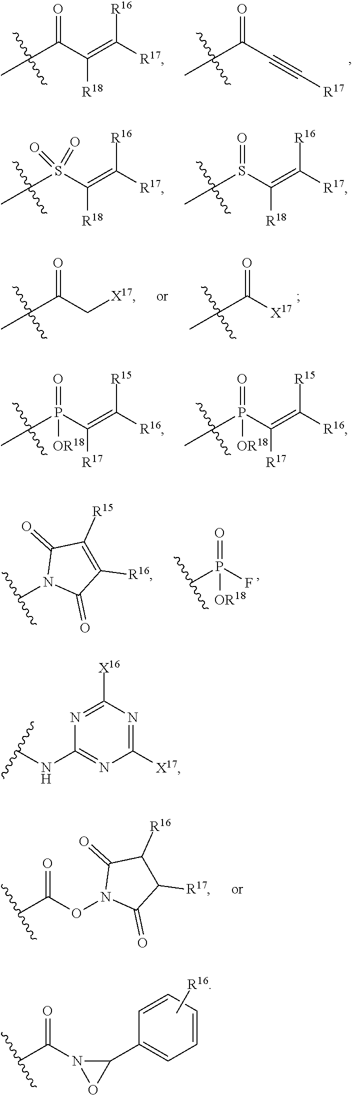

R¹⁵, R¹⁶, R¹⁷, R¹⁸, X¹⁶, and X¹⁷ are as described herein.

In embodiments, the first candidate compound contacted with a client protein including a solvent exposed reactive amino acid side chain, forms a client protein conjugate. In embodiments, the first candidate compound forms a covalent attachment to the solvent exposed reactive amino acid side chain of the client protein. The “client protein conjugate” includes a first candidate chemical moiety covalently bound to a solvent exposed reactive amino acid side chain of the client protein. In embodiments, the solvent exposed amino acid is cysteine and the candidate compound is a disulfide compound. In embodiments, a disulfide compound is an organic compound with disulfide moiety. In embodiments, the “client protein conjugate” is a product of a disulfide interchange reaction, where the first candidate chemical moiety is covalently bound to the client protein via a disulfide bond. The first candidate chemical moiety is tethered to the client protein via cysteine.

In embodiments, a conjugate-client complex includes a client protein and a protein conjugate. In embodiments, the conjugate-client complex is formed by contacting a protein conjugate with a client protein. In embodiments, the client protein is non-covalently bound to the protein conjugate. In embodiments, the client protein is covalently bound to the protein conjugate. In embodiments, a protein-client complex includes a protein and a client protein of said protein. In embodiments, the protein-client complex is formed by contacting a protein with a client protein.

In embodiments, a conjugate-protein complex includes a protein and a client protein conjugate. In embodiments, the conjugate-protein complex is formed by contacting a client protein conjugate with a protein. In embodiments, the protein is non-covalently bound to the client protein conjugate. In embodiments, the protein is covalently bound to the client protein conjugate. In embodiments, a protein-client complex includes a protein and a client protein of said protein. In embodiments, the protein-client complex is formed by contacting a protein with a client protein.

In embodiments, a first candidate compound contacted with a protein-client complex forms a conjugate-client complex. In embodiments, a first candidate chemical moiety is covalently attached to a solvent exposed reactive amino acid side chain on the protein of the protein-client complex.

In embodiments, a first candidate compound contacted with a protein-client complex forms a conjugate-protein complex. In embodiments, a first candidate chemical moiety is covalently attached to a solvent exposed reactive amino acid side chain on the client protein of the protein-client complex.

In embodiments, a compound that stabilizes binding of a protein to a client protein is selected from a disulfide library by screening. In embodiments, percent tethering of a chemical moiety to the protein is compared in the presence and in the absence of a client protein (or a phosphopeptide representing the protein binding motif of the client protein). The tethering of a chemical moiety to the protein yields a protein conjugate which can be detected by intact protein Mass Spectrometry (MS). “Percent tethering” is defined as the intensity of the protein conjugate peak divided by the sum of the intensities of all protein peaks, in the LC/MS spectra.

In embodiments, a compound that stabilizes binding of a protein to a client protein is not selected from a disulfide library by screening.

Some candidate compounds are tethered to the protein preferentially in the presence of the client protein (referred to herein as cooperative candidate compounds). Some candidate compounds are tethered to the protein only in the absence of the client protein (referred to herein as competitive candidate compounds). Some candidate compounds are tethered to the protein both in the presence or absence of the client protein (referred to herein as neutral candidate compounds). Overall percent tethering may also be determined. Chemical compounds exhibiting high overall tethering, and either neutral or cooperative behavior may be selected for further studies from the initial screen.

In embodiments, identified candidate compounds exhibit at least 50%, at least 55%, at least 60%, at least 65%, at least 70%, at least 75%, at least 80%, at least 85%, at least 90%, at least 95% or at least 99% overall percent tethering to the protein.

In embodiments, stability of the protein-client complex is assessed relative to stability of the conjugate-client complex. In embodiments, stability of the protein-client complex is assessed relative to stability of the conjugate-client complex using fluorescence anisotropy. In embodiments, a client protein is labeled with a fluorescent moiety (e.g., moiety of fluorescein) and contacted with the protein (e.g., the 14-3-3 protein). In embodiments, a client protein is labeled with a fluorescent moiety (e.g., moiety of fluorescein) and contacted with the protein conjugate. The apparent dissociation constants of the two complexes may be compared. In embodiments, the apparent dissociation constant of the conjugate-client complex is lower than the apparent dissociation constant of the protein-client complex (i.e., the conjugate-client complex exhibiting higher stability).

In embodiments, stability of the protein-client complex is assessed relative to stability of the conjugate-protein complex using fluorescence anisotropy. In embodiments, a protein is labeled with a fluorescent moiety (e.g., moiety of fluorescein) and contacted with a client protein. In embodiments, a protein is labeled with a fluorescent moiety (e.g., moiety of fluorescein) and contacted with a client protein conjugate. The apparent dissociation constants of the two complexes may be compared. In embodiments, the apparent dissociation constant of the conjugate-protein complex is lower than the apparent dissociation constant of the protein-client complex (i.e., conjugate-protein complex exhibiting higher stability).

In embodiments, the apparent dissociation constant of the conjugate-client complex is at least 2, 4, 8, 10, 20, 30, 40, 50, 60, 80, 100, 500, or 1000-fold lower than the apparent dissociation constant of the protein-client complex. In embodiments, the apparent dissociation constant of the conjugate-client complex is at least 2-fold lower than the apparent dissociation constant of the protein-client complex. In embodiments, the apparent dissociation constant of the conjugate-client complex is at least 4-fold lower than the apparent dissociation constant of the protein-client complex. In embodiments, the apparent dissociation constant of the conjugate-client complex is at least 8-fold lower than the apparent dissociation constant of the protein-client complex. In embodiments, the apparent dissociation constant of the conjugate-client complex is at least 10-fold lower than the apparent dissociation constant of the protein-client complex. In embodiments, the apparent dissociation constant of the conjugate-client complex is at least 20-fold lower than the apparent dissociation constant of the protein-client complex. In embodiments, the apparent dissociation constant of the conjugate-client complex is at least 30-fold lower than the apparent dissociation constant of the protein-client complex. In embodiments, the apparent dissociation constant of the conjugate-client complex is at least 40-fold lower than the apparent dissociation constant of the protein-client complex. In embodiments, the apparent dissociation constant of the conjugate-client complex is at least 50-fold lower than the apparent dissociation constant of the protein-client complex. In embodiments, the apparent dissociation constant of the conjugate-client complex is at least 60-fold lower than the apparent dissociation constant of the protein-client complex. In embodiments, the apparent dissociation constant of the conjugate-client complex is at least 80-fold lower than the apparent dissociation constant of the protein-client complex. In embodiments, the apparent dissociation constant of the conjugate-client complex is at least 100-fold lower than the apparent dissociation constant of the protein-client complex. In embodiments, the apparent dissociation constant of the conjugate-client complex is at least 500-fold lower than the apparent dissociation constant of the protein-client complex. In embodiments, the apparent dissociation constant of the conjugate-client complex is at least 1000-fold lower than the apparent dissociation constant of the protein-client complex.

In embodiments, the apparent dissociation constant of the conjugate-protein complex is at least 2, 4, 8, 10, 20, 30, 40, 50, 60, 80, 100, 500, or 1000-fold lower than the apparent dissociation constant of the protein-client complex. In embodiments, the apparent dissociation constant of the conjugate-protein complex is at least 2-fold lower than the apparent dissociation constant of the protein-client complex. In embodiments, the apparent dissociation constant of the conjugate-protein complex is at least 4-fold lower than the apparent dissociation constant of the protein-client complex. In embodiments, the apparent dissociation constant of the conjugate-protein complex is at least 8-fold lower than the apparent dissociation constant of the protein-client complex. In embodiments, the apparent dissociation constant of the conjugate-protein complex is at least 10-fold lower than the apparent dissociation constant of the protein-client complex. In embodiments, the apparent dissociation constant of the conjugate-protein complex is at least 20-fold lower than the apparent dissociation constant of the protein-client complex. In embodiments, the apparent dissociation constant of the conjugate-protein complex is at least 30-fold lower than the apparent dissociation constant of the protein-client complex. In embodiments, the apparent dissociation constant of the conjugate-protein complex is at least 40-fold lower than the apparent dissociation constant of the protein-client complex. In embodiments, the apparent dissociation constant of the conjugate-protein complex is at least 50-fold lower than the apparent dissociation constant of the protein-client complex. In embodiments, the apparent dissociation constant of the conjugate-protein complex is at least 60-fold lower than the apparent dissociation constant of the protein-client complex. In embodiments, the apparent dissociation constant of the conjugate-protein complex is at least 80-fold lower than the apparent dissociation constant of the protein-client complex. In embodiments, the apparent dissociation constant of the conjugate-protein complex is at least 100-fold lower than the apparent dissociation constant of the protein-client complex. In embodiments, the apparent dissociation constant of the conjugate-protein complex is at least 500-fold lower than the apparent dissociation constant of the protein-client complex. In embodiments, the apparent dissociation constant of the conjugate-protein complex is at least 1000-fold lower than the apparent dissociation constant of the protein-client complex.

In embodiments, the protein is a “hub” protein. A “hub” protein refers to a protein that interacts with a number of different proteins in a protein-protein interaction networks. Hubs can be static or dynamic. Static hubs bind a large number of partners simultaneously at different sites, for example, BRCA2. Dynamic hubs bind multiple partners that compete for the same site. Well-known examples of dynamic hubs include calmodulin, dynein light chain LC8, and 14-3-3 proteins.

In embodiments, the protein is a 14-3-3 protein. The 14-3-3 isoforms are highly homologous in the primary phosphopeptide binding groove. In embodiments, the term refers to the c isoform.

In embodiments, the solvent exposed reactive amino acid side chain of the 14-3-3 protein is the side chain of a cysteine, methionine, tryptophan, tyrosine, lysine or histidine. In embodiments, the solvent exposed reactive amino acid side chain of the 14-3-3 protein is the side chain of a cysteine. In embodiments, the solvent exposed reactive amino acid side chain of the 14-3-3 protein is thiol.

In embodiments, the solvent exposed reactive amino acid side chain of the 14-3-3 protein is the side chain of a methionine, tryptophan, tyrosine, lysine or histidine. In embodiments, the solvent exposed reactive amino acid side chain of the 14-3-3 protein is not the side chain of a cysteine. In embodiments, the solvent exposed reactive amino acid side chain of the 14-3-3 protein is not thiol.

In embodiments, the solvent exposed reactive amino acid side chain of the 14-3-3 client protein is the side chain of a cysteine, methionine, tryptophan, tyrosine, lysine or histidine. In embodiments, the solvent exposed reactive amino acid side chain of the 14-3-3 client protein is the side chain of a cysteine. In embodiments, the solvent exposed reactive amino acid side chain of the 14-3-3 client protein is thiol.

In embodiments, the solvent exposed reactive amino acid side chain of the 14-3-3 protein is proximal to a 14-3-3 client protein binding site. In embodiments, a solvent exposed reactive amino acid proximal to a 14-3-3 client protein binding site is C38, N42, S45, V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, a solvent exposed reactive amino acid proximal to a 14-3-3 client protein binding site is C38, N42, S45, V46, E115, F119, K122, D126, P167, I168, G171, L172, L174, N175, or I219 (e.g., of 14-3-3σ). In embodiments, the solvent exposed reactive amino acid proximal to the 14-3-3 client binding site corresponds to C38, N42, S45, V46, E115, F119, K122, D126, P167, I168, G171, L172, L174, N175, I219, E39, R56, R60, Y130, E133, V178, E182, L222, D225, N226, and L229 (e.g., of 14-3-3σ). In embodiments, the solvent exposed reactive amino acid proximal to the 14-3-3 client binding site corresponds to C38, N42, S45, V46, E115, F119, K122, D126, P167, I168, G171, L172, L174, N175, or I219 (e.g., of 14-3-3σ).

In embodiments, a solvent exposed reactive amino acid proximal to a 14-3-3 client protein binding site is N42, S45, V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, a solvent exposed reactive amino acid proximal to a 14-3-3 client protein binding site is N42, S45, V46, E115, F119, K122, D126, P167, I168, G171, L172, L174, N175, or I219 (e.g., of 14-3-3σ). In embodiments, the solvent exposed reactive amino acid proximal to the 14-3-3 client binding site corresponds to N42, S45, V46, E115, F119, K122, D126, P167, I168, G171, L172, L174, N175, I219, E39, R56, R60, Y130, E133, V178, E182, L222, D225, N226, and L229 (e.g., of 14-3-3σ). In embodiments, the solvent exposed reactive amino acid proximal to the 14-3-3 client binding site corresponds to N42, S45, V46, E115, F119, K122, D126, P167, I168, G171, L172, L174, N175, or I219 (e.g., of 14-3-3σ). In embodiments, a solvent exposed reactive amino acid proximal to a 14-3-3 client protein binding site is V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, a solvent exposed reactive amino acid proximal to a 14-3-3 client protein binding site is V46, E115, F119, K122, D126, P167, I168, G171, L172, L174, N175, or I219 (e.g., of 14-3-3σ). In embodiments, the solvent exposed reactive amino acid proximal to the 14-3-3 client binding site corresponds to V46, E115, F119, K122, D126, P167, I168, G171, L172, L174, N175, I219, E39, R56, R60, Y130, E133, V178, E182, L222, D225, N226, and L229 (e.g., of 14-3-3σ). In embodiments, the solvent exposed reactive amino acid proximal to the 14-3-3 client binding site corresponds to V46, E115, F119, K122, D126, P167, I168, G171, L172, L174, N175, or I219 (e.g., of 14-3-3σ).

In embodiments, the 14-3-3 protein includes an amino acid mutation. In embodiments, the 14-3-3σ protein includes a mutation of amino acids corresponding to C38, N42, S45, V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, the 14-3-3σ protein includes a mutation of amino acids corresponding to C38, N42, S45, V46, E115, F119, K122, D126, P167,1168, G171, L172, L174, N175, or I219 (e.g., of 14-3-3σ). In embodiments, the 14-3-3σ protein includes a mutation of amino acid corresponding to C38. In embodiments, the 14-3-3σ protein includes a mutation of amino acid corresponding to N42. In embodiments, the 14-3-3σ protein includes a mutation of amino acid corresponding to S45. In embodiments, the 14-3-3σ protein includes a mutation of amino acid corresponding to V46. In embodiments, the 14-3-3σ protein includes a mutation of amino acid corresponding to E115. In embodiments, the 14-3-3β protein includes a mutation of amino acid corresponding to F119. In embodiments, the 14-3-3σ protein includes a mutation of amino acid corresponding to K122. In embodiments, the 14-3-3σ protein includes a mutation of amino acid corresponding to D126. In embodiments, the 14-3-3σ protein includes a mutation of amino acid corresponding to P167. In embodiments, the 14-3-3σ protein includes a mutation of amino acid corresponding to 1168. In embodiments, the 14-3-3σ protein includes a mutation of amino acid corresponding to G171. In embodiments, the 14-3-3σ protein includes a mutation of amino acid corresponding to L172. In embodiments, the 14-3-3σ protein includes a mutation of amino acid corresponding to L174. In embodiments, the 14-3-3σ protein includes a mutation of amino acid corresponding to N175. In embodiments, the 14-3-3σ protein includes a mutation of amino acid corresponding to I219. In embodiments, the 14-3-3σ protein includes a mutation of amino acid corresponding to E39. In embodiments, the 14-3-3σ protein includes a mutation of amino acid corresponding to R56. In embodiments, the 14-3-3σ protein includes a mutation of amino acid corresponding to R60. In embodiments, the 14-3-3σ protein includes a mutation of amino acid corresponding to Y130. In embodiments, the 14-3-3σ protein includes a mutation of amino acid corresponding to E133. In embodiments, the 14-3-3σ protein includes a mutation of amino acid corresponding to V178. In embodiments, the 14-3-3σ protein includes a mutation of amino acid corresponding to E182. In embodiments, the 14-3-3σ protein includes a mutation of amino acid corresponding to L222. In embodiments, the 14-3-3σ protein includes a mutation of amino acid corresponding to D225. In embodiments, the 14-3-3σ protein includes a mutation of amino acid corresponding to N226. In embodiments, the 14-3-3σ protein includes a mutation of amino acid corresponding to L229. In embodiments, the 14-3-3σ protein includes a mutation of an amino acid capable of contacting a client protein. In embodiments, the 14-3-3σ protein includes a mutation of an amino acid adjacent (e.g., in primary sequence or in three dimensional space when the protein is folded) to an amino acid corresponding to V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, the 14-3-3σ protein includes a mutation of an amino acid adjacent (e.g., in primary sequence or in three dimensional space when the protein is folded) to an amino acid capable of contacting a client protein.

In embodiments, the 14-3-3 protein includes an amino acid mutation. In embodiments, the 14-3-3σ protein includes a mutation of amino acids corresponding to V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, the 14-3-3σ protein includes a mutation of amino acids corresponding to V46, E115, F119, K122, D126, P167, I168, G171, L172, L174, N175, or I219 (e.g., of 14-3-3σ). In embodiments, the 14-3-3σ protein does not include a mutation of amino acid corresponding to C38 (e.g., of 14-3-3σ). In embodiments, the 14-3-3σ protein does not include a mutation of amino acid corresponding to N42 (e.g., of 14-3-3σ). In embodiments, the 14-3-3σ protein does not include a mutation of amino acid corresponding to S45 (e.g., of 14-3-3σ). In embodiments, the 14-3-3 protein includes a mutation of an amino acid capable of contacting a client protein. In embodiments, the 14-3-3 protein includes a mutation of an amino acid adjacent (e.g., in primary sequence or in three dimensional space when the protein is folded) to an amino acid corresponding to V46, E115, F119, K122, D126, P167, I168, G171, L172, L174, N175, I219, E39, R56, R60, Y130, E133, V178, E182, L222, D225, N226, or L229 (e.g., of 14-3-3σ). In embodiments, the 14-3-3 protein includes a mutation of an amino acid adjacent (e.g., in primary sequence or in three dimensional space when the protein is folded) to an amino acid capable of contacting a client protein.

In embodiments, amino acids corresponding to N42, S45, V46, E115, F119, K122, D126, P167, I168, G171, L172, L174, N175, I219, E39, R56, R60, Y130, E133, V178, E182, L222, D225, N226, or L229 (e.g., of 14-3-3σ) are mutated to a Cysteine (Cys, C). In embodiments, amino acids corresponding to N42, S45, V46, E115, F119, K122, D126, P167, 1168, G171, L172, L174, N175, I219, E39, R56, R60, Y130, E133, V178, E182, L222, D225, N226, or L229 (e.g. of 14-3-3σ) are mutated to a Cysteine (Cys, C) and amino acid corresponding to C38 is mutated to Asparagine (Asn, N).

In embodiments, amino acids corresponding to V46, E115, F119, K122, D126, P167, I168, G171, L172, L174, N175, I219, E39, R56, R60, Y130, E133, V178, E182, L222, D225, N226, or L229 (e.g. of 14-3-3σ) are mutated to a Cysteine (Cys, C). In embodiments, amino acids corresponding to V46, E115, F119, K122, D126, P167, I168, G171, L172, L174, N175, I219, E39, R56, R60, Y130, E133, V178, E182, L222, D225, N226, or L229 (e.g. of 14-3-3σ) are mutated to a Cysteine (Cys, C) and amino acid corresponding to C38 (e.g. of 14-3-3σ) is mutated to Asparagine (Asn, N).

In embodiments, the 14-3-3σ protein includes a mutation of amino acid corresponding to Asparagine (Asn, N) 42 to Cysteine (Cys, C) and amino acid corresponding to Cysteine 38 (Cys, C) to Asparagine (Asn, N). In embodiments, the 14-3-3σ protein includes a mutation of amino acid corresponding to Serine (Ser, S) 45 to Cysteine (Cys, C) and amino acid corresponding to Cysteine 38 (Cys, C) to Asparagine (Asn, N). In embodiments, the 14-3-3σ protein includes a mutation of amino acid corresponding to Valine (Val, V) 45 to Cysteine (Cys, C) and amino acid corresponding to Cysteine 38 (Cys, C) to Asparagine (Asn, N). In embodiments, the 14-3-3c protein includes a mutation of amino acid corresponding to Glutamic Acid (Glu, E) 115 to Cysteine (Cys, C) and amino acid corresponding to Cysteine 38 (Cys, C) to Asparagine (Asn, N). In embodiments, the 14-3-3σ protein includes a mutation of amino acid corresponding to Phenylalanine (Phe, F) 119 to Cysteine (Cys, C) and amino acid corresponding to Cysteine 38 (Cys, C) to Asparagine (Asn, N). In embodiments, the 14-3-3σ protein includes a mutation of amino acid corresponding to Lysine (Lys, K) 122 to Cysteine (Cys, C) and amino acid corresponding to Cysteine 38 (Cys, C) to Asparagine (Asn, N). In embodiments, the 14-3-3; protein includes a mutation of amino acid corresponding to Aspartic acid (Asp, D) 126 to Cysteine (Cys, C) and amino acid corresponding to Cysteine 38 (Cys, C) to Asparagine (Asn, N). In embodiments, the 14-3-3σ protein includes a mutation of amino acid corresponding to Proline (Pro, P) 167 to Cysteine (Cys, C) and amino acid corresponding to Cysteine 38 (Cys, C) to Asparagine (Asn, N). In embodiments, the 14-3-3σ protein includes a mutation of amino acid corresponding to Isoleucine (Ile, I) 168 to Cysteine (Cys, C) and amino acid corresponding to Cysteine 38 (Cys, C) to Asparagine (Asn, N). In embodiments, the 14-3-3c protein includes a mutation of amino acid corresponding to Glycine (Gly, G) 171 to Cysteine (Cys, C) and amino acid corresponding to Cysteine 38 (Cys, C) to Asparagine (Asn, N). In embodiments, the 14-3-3σ protein includes a mutation of amino acid corresponding to Leucine (Leu, L) 172 to Cysteine (Cys, C) and amino acid corresponding to Cysteine 38 (Cys, C) to Asparagine (Asn, N). In embodiments, the 14-3-3σ protein includes a mutation of amino acid corresponding to Leucine (Leu, L) 174 to Cysteine (Cys, C) and amino acid corresponding to Cysteine 38 (Cys, C) to Asparagine (Asn, N). In embodiments, the 14-3-3c protein includes a mutation of amino acid corresponding to Asparagine (Asn, N) 175 to Cysteine (Cys, C) and amino acid corresponding to Cysteine 38 (Cys, C) to Asparagine (Asn, N). In embodiments, the 14-3-3σ protein includes a mutation of amino acid corresponding to Isoleucine (Ile, I) 219 to Cysteine (Cys, C) and amino acid corresponding to Cysteine 38 (Cys, C) to Asparagine (Asn, N).

In embodiments, the 14-3-3σ protein does not include a mutation of amino acid corresponding to Asparagine (Asn, N) 42 to Cysteine (Cys, C) and amino acid corresponding to Cysteine 38 (Cys, C) to Asparagine (Asn, N). In embodiments, the 14-3-3β protein does not include a mutation of amino acid corresponding to Serine (Ser, S) 45 to Cysteine (Cys, C) and amino acid corresponding to Cysteine 38 (Cys, C) to Asparagine (Asn, N).

In embodiments, the 14-3-3σ protein includes a mutation of amino acid corresponding to Glutamic acid (Glu, E) 39 to Cysteine (Cys, C) and amino acid corresponding to Cysteine 38 (Cys, C) to Asparagine (Asn, N). In embodiments, the 14-3-3σ protein includes a mutation of amino acid corresponding to Arginine (Arg, R) 56 to Cysteine (Cys, C) and amino acid corresponding to Cysteine 38 (Cys, C) to Asparagine (Asn, N). In embodiments, the 14-3-3σ protein includes a mutation of amino acid corresponding to Arginine (Arg, R) 60 to Cysteine (Cys, C) and amino acid corresponding to Cysteine 38 (Cys, C) to Asparagine (Asn, N). In embodiments, the 14-3-3σ protein includes a mutation of amino acid corresponding to Tyrosine (Tyr, Y) 130 to Cysteine (Cys, C) and amino acid corresponding to Cysteine 38 (Cys, C) to Asparagine (Asn, N). In embodiments, the 14-3-3σ protein includes a mutation of amino acid corresponding to Glutamic acid (Glu, E) 133 to Cysteine (Cys, C) and amino acid corresponding to Cysteine 38 (Cys, C) to Asparagine (Asn, N). In embodiments, the 14-3-3σ protein includes a mutation of amino acid corresponding to Valine (Val, V) 178 to Cysteine (Cys, C) and amino acid corresponding to Cysteine 38 (Cys, C) to Asparagine (Asn, N). In embodiments, the 14-3-3σ protein includes a mutation of amino acid corresponding to Glutamic acid (Glu, E) 182 to Cysteine (Cys, C) and amino acid corresponding to Cysteine 38 (Cys, C) to Asparagine (Asn, N). In embodiments, the 14-3-3σ protein includes a mutation of amino acid corresponding to Leucine (Leu, L) 222 to Cysteine (Cys, C) and amino acid corresponding to Cysteine 38 (Cys, C) to Asparagine (Asn, N). In embodiments, the 14-3-3σ protein includes a mutation of amino acid corresponding to Aspartic acid (Asp, D) 225 to Cysteine (Cys, C) and amino acid corresponding to Cysteine 38 (Cys, C) to Asparagine (Asn, N). In embodiments, the 14-3-3σ protein includes a mutation of amino acid corresponding to Aspargine (Asn, N) 226 to Cysteine (Cys, C) and amino acid corresponding to Cysteine 38 (Cys, C) to Asparagine (Asn, N). In embodiments, the 14-3-3σ protein includes a mutation of amino acid corresponding to Leucine (Leu, L) 229 to Cysteine (Cys, C) and amino acid corresponding to Cysteine 38 (Cys, C) to Asparagine (Asn, N). In embodiments, the 14-3-3 protein includes an amino acid mutation of the amino acid corresponding to C38, N42, S45, V46, E115, F119, K122, D126, P167, I168, G171, L172, L174, N175, I219, E39, R56, R60, Y130, E133, V178, E182, L222, D225, N226, or L229 of 14-3-3σ. In embodiments, the 14-3-3 protein includes a mutation of an amino acid corresponding to C38, N42, S45, V46, E115, F119, K122, D126, P167, I168, G171, L172, L174, N175, or I219 of 14-3-3σ. In embodiments, the 14-3-3 protein includes a mutation (e.g., to Asn, Lys, Met, His, Trp, or Tyr) of amino acid corresponding to C38 of 14-3-3σ. In embodiments, the 14-3-3 protein includes a mutation (e.g., to Cys, Lys, Met, His, Trp, or Tyr) of amino acid corresponding to N42 of 14-3-3σ. In embodiments, the 14-3-3 protein includes a mutation (e.g., to Cys, Lys, Met, His, Trp, or Tyr) of amino acid corresponding to S45 of 14-3-3σ. In embodiments, the 14-3-3 protein includes a mutation (e.g., to Cys, Lys, Met, His, Trp, or Tyr) of amino acid corresponding to V46 of 14-3-3σ. In embodiments, the 14-3-3 protein includes a mutation (e.g., to Cys, Lys, Met, His, Trp, or Tyr) of amino acid corresponding to E115 of 14-3-3σ. In embodiments, the 14-3-3 protein includes a mutation (e.g., to Cys, Lys, Met, His, Trp, or Tyr) of amino acid corresponding to F119 of 14-3-3σ. In embodiments, the 14-3-3 protein includes a mutation (e.g., to Cys, Met, His, Trp, or Tyr) of amino acid corresponding to K122 of 14-3-3σ. In embodiments, the 14-3-3 protein includes a mutation (e.g., to Cys, Lys, Met, His, Trp, or Tyr) of amino acid corresponding to D126 of 14-3-3σ. In embodiments, the 14-3-3 protein includes a mutation (e.g., to Cys, Lys, Met, His, Trp, or Tyr) of amino acid corresponding to P167 of 14-3-3σ. In embodiments, the 14-3-3 protein includes a mutation (e.g., to Cys, Lys, Met, His, Trp, or Tyr) of amino acid corresponding to 1168 of 14-3-3σ. In embodiments, the 14-3-3 protein includes a mutation (e.g., to Cys, Lys, Met, His, Trp, or Tyr) of amino acid corresponding to G171 of 14-3-3σ. In embodiments, the 14-3-3 protein includes a mutation (e.g., to Cys, Lys, Met, His, Trp, or Tyr) of amino acid corresponding to L172 of 14-3-3σ. In embodiments, the 14-3-3 protein includes a mutation (e.g., to Cys, Lys, Met, His, Trp, or Tyr) of amino acid corresponding to L174 of 14-3-3σ. In embodiments, the 14-3-3 protein includes a mutation (e.g., to Cys, Lys, Met, His, Trp, or Tyr) of amino acid corresponding to N175 of 14-3-3n. In embodiments, the 14-3-3 protein includes a mutation (e.g., to Cys, Lys, Met, His, Trp, or Tyr) of amino acid corresponding to I219 of 14-3-3σ. In embodiments, the 14-3-3 protein includes a mutation (e.g., to Cys, Lys, Met, His, Trp, or Tyr) of amino acid corresponding to E39 of 14-3-3σ. In embodiments, the 14-3-3 protein includes a mutation (e.g., to Cys, Lys, Met, His, Trp, or Tyr) of amino acid corresponding to R56 of 14-3-3σ. In embodiments, the 14-3-3 protein includes a mutation (e.g., to Cys, Lys, Met, His, Trp, or Tyr) of amino acid corresponding to R60 of 14-3-3σ. In embodiments, the 14-3-3 protein includes a mutation (e.g., to Cys, Lys, Met, His, or Trp) of amino acid corresponding to Y130 of 14-3-3σ. In embodiments, the 14-3-3 protein includes a mutation (e.g., to Cys, Lys, Met, His, Trp, or Tyr) of amino acid corresponding to E133 of 14-3-3σ. In embodiments, the 14-3-3 protein includes a mutation (e.g., to Cys, Lys, Met, His, Trp, or Tyr) of amino acid corresponding to V178 of 14-3-3σ). In embodiments, the 14-3-3 protein includes a mutation (e.g., to Cys, Lys, Met, His, Trp, or Tyr) of amino acid corresponding to E182 of 14-3-3σ. In embodiments, the 14-3-3 protein includes a mutation (e.g., to Cys, Lys, Met, His, Trp, or Tyr) of amino acid corresponding to L222 of 14-3-3σ. In embodiments, the 14-3-3 protein includes a mutation (e.g., to Cys, Lys, Met, His, Trp, or Tyr) of amino acid corresponding to D225 of 14-3-3σ. In embodiments, the 14-3-3 protein includes a mutation (e.g., to Cys, Lys, Met, His, Trp, or Tyr) of amino acid corresponding to N226 of 14-3-3σ. In embodiments, the 14-3-3 protein includes a mutation (e.g., to Cys, Lys, Met, His, Trp, or Tyr) of amino acid corresponding to L229 of 14-3-3σ. In embodiments the amino acid may be mutated to any other natural amino acid.

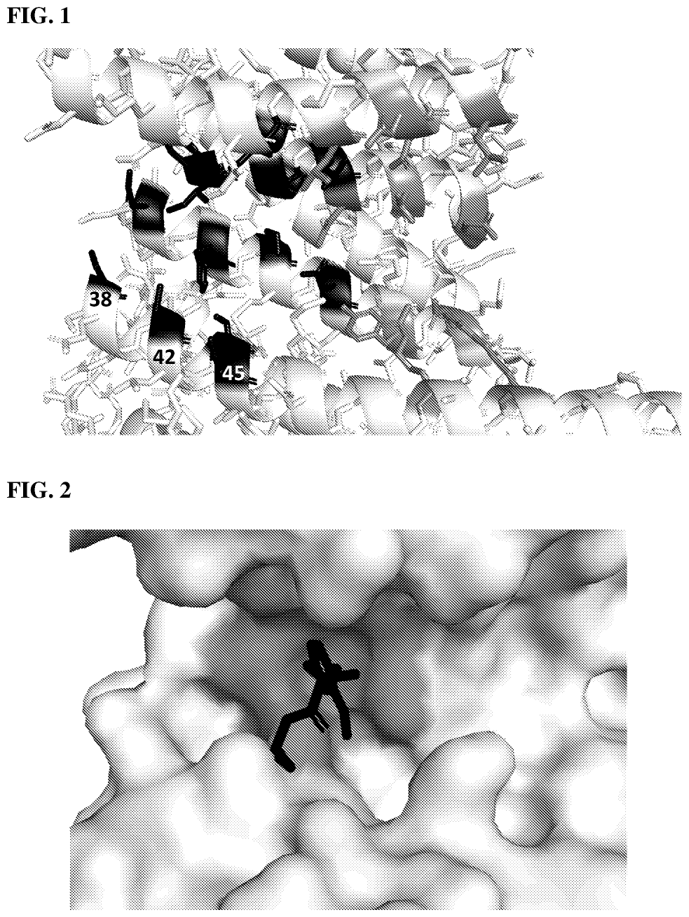

In embodiments, residues that can be mutated for identifying a chemical compound that stabilizes binding of the 14-3-3σ protein to its client protein are depicted in FIG. 1. In embodiments, the 14-3-3 client protein is phosphorylated. In embodiments, the 14-3-3 client is a phosphoserine protein. In embodiments, the 14-3-3 client is a phosphothreonone protein. In embodiments, the 14-3-3 client is a phosphothreonine protein. In embodiments, the 14-3-3 client is a phosphorylated peptide (a phosphopeptide) derived from the 14-3-3 client protein. In embodiments, the 14-3-3 client is a phosphorylated peptide (phosphopeptide) representing the 14-3-3 protein binding motif of the client protein.

In embodiments, the chemical compound that stabilizes binding of the 14-3-3σ protein to its client protein contacts a 14-3-3c protein amino acid corresponding to F119, K122, P167, I168, G171, and L172. In embodiments, the chemical compound that stabilizes binding of the 14-3-3σ protein to its client protein contacts a 14-3-3β protein amino acid corresponding to F119. In embodiments, the chemical compound that stabilizes binding of the 14-3-3σ protein to its client protein contacts a 14-3-3σ protein amino acid corresponding to K122. In embodiments, the chemical compound that stabilizes binding of the 14-3-3σ protein to its client protein contacts a 14-3-3c protein amino acid corresponding to P167. In embodiments, the chemical compound that stabilizes binding of the 14-3-3c protein to its client protein contacts a 14-3-3σ protein amino acid corresponding to 1168. In embodiments, the chemical compound that stabilizes binding of the 14-3-3c protein to its client protein contacts a 14-3-3c protein amino acid corresponding to G171. In embodiments, the chemical compound that stabilizes binding of the 14-3-3σ protein to its client protein contacts a 14-3-3σ protein amino acid corresponding to L192. Said amino acids are in a binding pocket determined to be a “hot spot”, which is depicted in FIG. 2. In embodiments, a 14-3-3c client protein is stabilized by the identified chemical compound, irrespective of peptide structure.

In embodiments, the chemical moieties that stabilize binding of a 14-3-3 (e.g., the 14-3-3σ) protein to its client protein are:

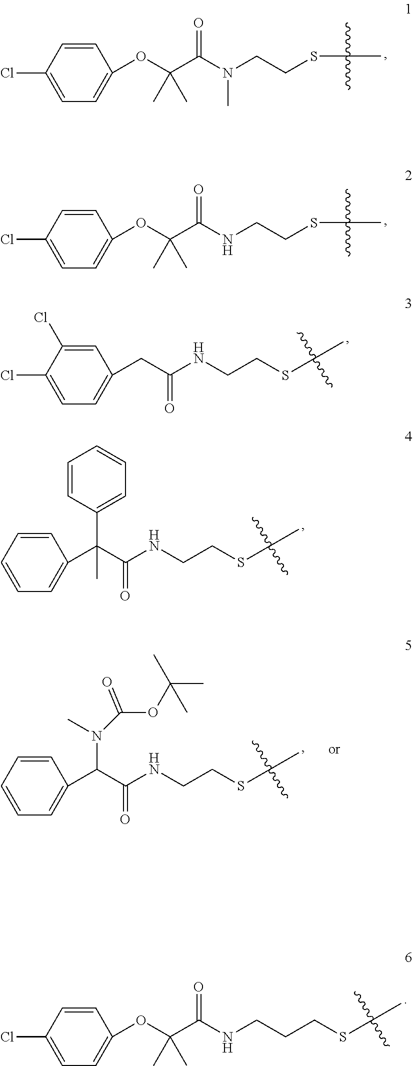

In embodiments, the chemical moieties that stabilize binding of a 14-3-3 (e.g. the 14-3-3σ) protein to its client protein are not: (e.g., Entrez 7531, UniProt P62258, RefSeq NP_6752)

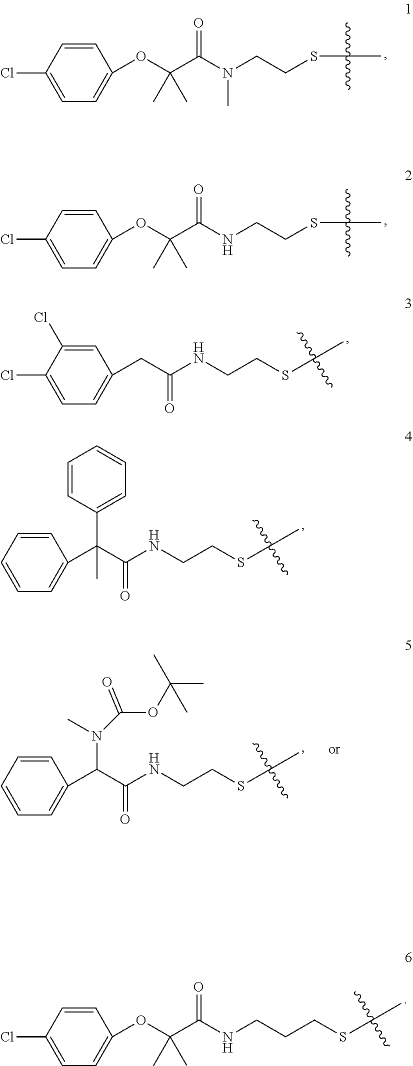

In embodiments, 14-3-3 client protein or 14-3-3 client is phosphorylated. In embodiments, the 14-3-3σ client protein or 14-3-3σ client is phosphorylated. In embodiments, the 14-3-3 client protein is a phosphoserine protein. In embodiments, the 14-3-3 client protein is a phosphothreonine protein. In embodiments, the 14-3-3 client is a phosphorylated peptide (a phosphopeptide) derived from the 14-3-3 client protein. In embodiments, the 14-3-3 client is a phosphorylated peptide (phosphopeptide) representing the 14-3-3 protein binding motif of the client protein.

In embodiments, the 14-3-3 client protein and the indication associated with said client protein, are listed in Table 1. The 14-3-3 client proteins listed in Table 1 are useful in the methods of identifying a chemical compound that modulates the binding of a protein to a client protein, as set forth herein.

TABLE 1

| Name of 14-3-3 | | |
|---|---|---|
| Client Protein | Indication | Literature |
| 14-3-3-FAM22A fusion | Cancer | 1-3, 3-8 |
| AANAT | Neuro | 9, 9, 9-13, 13-17 |
| AMOT130 | Cancer | 18 |
| AS160 | Diabetes | 19-24, 24-29 |
| ASK1 | Neuro | 30-38 |
| AUF1 | Neuro | 39-41 |
| B,C-Raf | Cancer | 42-59 |
| BAD | Neurodeg | 60-72 |
| BAG3 | Neurodeg | 73-75 |
| BAP1 | Cancer | 76 |
| Bax | Neurodeg. | 77-82 |
| Bid | Neurodeg | 83, 84 |
| Bim | Neurodeg | 85 |
| BLNK | Inflammation | 86, 87 |
| BTK | Cancer | 88 |
| CaMKK2 | | 89 |
| CaV2.2 | Neuro | 90-92 |
| Casp2 | | 93-96 |
| Cdc25B,C | Cancer | 97-119 |
| CDK2 | Cancer | 120 |
| CFTR | Cystic Fibrosis | 121-125 |
| Chibby | Cancer | 126-133 |
| CHK1 | Cancer | 134-137 |
| ChREBP | Metabolic Dis. | 138-142 |
| CSP | Neuro | 143-145 |
| CyaA | | 146 |
| E2F1 | Cancer | 147, 148 |
| ERalpha (ERa) | Cancer | 149 |
| Exoenzyme S (ExoS) | Infection | 150-172 |
| Exoenzyme T | Infection | 173 |
| FOXO-1 | Diabetes/Neuro | 174-184 |
| GAB2 | Cancer | 185-188 |
| GAKIN/KIF13B | Neurodeg | 189 |
| Gli1 | | 190 |
| GP120 | | 191-193 |
| HAP1 | Neurodeg | 194 |
| HBX | | 195 |
| HCV | | 196-199 |
| HDAC4,5,7 | Cancer | 200-210 |
| Histone H3 | | 211, 211-217 |
| HSF1 | Cancer | 218, 219 |
| HSPB6 | | 220-226 |
| Huntingtin | Huntington | 194, 227 |
| Integrin α4 | | 228 |
| Integrin β2 | | 228, 228-232 |
| IGFR | Diabetes | 233-238 |
| IL3-R | Inflammation | 239, 240 |
| IL9-R | Inflammation | 241, 242 |
| IkB | Inflammation | 243 |
| IRSp53 | | 244 |
| IRS1,2 | Diabetes | 233, 245-250 |
| Jun | Cancer | 251 |
| KSR | Cancer | 252-258 |
| KSR | Cancer | 204, 205, 207, |
| | | 209-21268, 69, 71, |
| | | 73, 74, 125, 126 |
| LASP1 | Cancer | 261 |
| LFA-1 | Inflammation | 229-231, 262-264 |
| LKB1 | | 265-267 |
| LRRK2 | Parkinson | 268-283 |
| MDM2 | Cancer | 284-286 |
| MDMX | Cancer | 287-294 |
| MLF1 | Cancer | 295-297 |
| MondoA | Metabol. Dis. | 298, 299 |
| MondoB/ChREBP | Metabol. Dis. | 138-142, 300-302 |
| MST4 | | |
| MT | | 303-307 |
| Myc | Cancer | 308, 309 |
| Myo1C | Metabol. Dis. | 310-313 |
| Ndel | Miller-Dieker | 314, 315 |
| | syndrome | |
| NELFE | Cancer | 316 |
| NFAT | Inflammation | 317, 318 |
| NFκB | Inflammation | 243, 319, 320 |
| NHE1 | Cancer | 321-324 |
| Notch4 | Cancer | 325 |
| NS1 | | 326 |
| PADI6 | | 327, 328 |
| PAK6 | | 329 |
| PI4KIIIB | | |
| p27 | Cancer | 330-335 |
| p53 | Cancer | 284, 336-340 |
| PlexA | Neurodeg | 341 |
| PRAS40 | Metabol. Dis. | 342-349, 349-351 |
| PrP | Neurodeg | 352, 353 |
| Pyrin | Inflammation | 354-359 |
| RapGEF2 | | |
| Raptor | Metabol. Dis. | 360, 361 |
| REDD1 | Diabetes | 251, 362-366 |
| Rem2 | Neuro | 367 |
| RIG-I | Infection/ | 368-372 |
| | inflammation | |
| RND3 | | 373 |
| STARD1 | | 374, 375 |
| Shroom3 | Chronic Kidney | 376 |
| | Disease (CKD) | |
| SLP76 | Inflammation | 377 |
| Snail | Cancer | |
| SOS1 | Cancer | 378-380 |
| SRPK2 | Neuro | 381, 382 |
| Synaptopodin | Diabetes | 383-385 |
| TASK1,3 | Neuro | 386-390 |
| Tau | Alzheimer | 391-396, 396-402, |
| | | 402-406 |
| TBC1D1 | Diabetes | 407, 408 |
| TBC1D7 | Diabetes | 409 |
| TFEB | Autophagy | 410 |
| TERT | Cancer | 411-414 |
| TET2 | Cancer | 415, 416 |
| TGase2 | Neuro | 417-419 |
| TH | Neurodeg | 420-423 |
| TPH | Neurodeg | 420, 420-423 |
| TSC2 | Diabetes | 424-428 |
| USP8 | Cushing’s Disease | 429 |
| Vpr | | 430-437 |
| YAP/TAZ | Cancer | 18, 18, 438-440, |
| | | 440-442, 442-453, |
| | | 453-456 |
| αII spectrin | Neurodeg | 457 |
| α-Synuclein | Parkinson | 458-464 |

In embodiments, the 14-3-3 client protein is ERα, ERRγ, TASK3, ExoS, MYC, Rel A, FOXO-1 or TAZ or functional replacement thereof. In embodiments, the 14-3-3 client protein is ERα or functional replacement thereof. In embodiments, the 14-3-3 client protein is ERRγ or functional replacement thereof. In embodiments, the 14-3-3 client protein is TASK3 or functional replacement thereof. In embodiments, the 14-3-3 client protein is ExoS or functional replacement thereof. In embodiments, the 14-3-3 client protein is MYC or functional replacement thereof. In embodiments, the 14-3-3 client protein is Rel A or functional replacement thereof. In embodiments, the 14-3-3 client protein is FOXO-1 or functional replacement thereof. In embodiments, the 14-3-3 client protein is TAZ or functional replacement thereof.

In embodiments, the 14-3-3 client protein is ERα, ERRγ, TASK3, ExoS, MYC, Rel A, FOXO-1, Cdc25A, Cdc25B, Cdc25C, Cdc2, Wee1, E2F1, ARaf, BRaf, CRaf, SLP76, BLNK, Mdm2, MdmX, PKR, RIPK2, NPM1, Pyrin, ChREBP, CIP2A, DAPK2, LDB1, MAGI1, NDE1, RND3, SSBP2, SSBP3, SSBP4, MLF1, RapGEF2, p53, Shroom3, Casp2, Cby, Tau, Ataxin1, IKBa, CFTR, TBC1D7, Gab2, USP8, SOS1, PAK6, CaMKK2, IntB2, IntAlpha4, ASKI, LRRK2, YAP or TAZ or functional replacement thereof. In embodiments, the 14-3-3 client protein is ERα or functional replacement thereof. In embodiments, the 14-3-3 client protein is ERRγ or functional replacement thereof. In embodiments, the 14-3-3 client protein is TASK3 or functional replacement thereof. In embodiments, the 14-3-3 client protein is ExoS or functional replacement thereof. In embodiments, the 14-3-3 client protein is MYC or functional replacement thereof. In embodiments, the 14-3-3 client protein is Rel A or functional replacement thereof. In embodiments, the 14-3-3 client protein is FOXO-1 or functional replacement thereof. In embodiments, the 14-3-3 client protein is TAZ or functional replacement thereof.

In embodiments, the 14-3-3 client protein is ERRγ, TASK3, ExoS, MYC, Rel A, FOXO-1 or TAZ or functional replacement thereof. In embodiments, the 14-3-3 client protein is TASK3, ExoS, MYC, Rel A, FOXO-1 or TAZ or functional replacement thereof. In embodiments, the 14-3-3 client protein is not ERα or functional replacement thereof. In embodiments, the 14-3-3 client protein is not ERRγ or functional replacement thereof.

“ERRγ” refers to a nuclear receptor that in humans is encoded by the ESRRG (EStrogen Related Receptor Gamma) gene. A nuclear receptor is a protein found within cells responsible for sensing steroid and thyroid hormones and certain other molecules. In response, these receptors work with other proteins to regulate the expression of specific genes thereby controlling the development, homeostasis and metabolism of the organism. This receptor is classified as transcription factor. A transcription factor (TF) is a protein that controls the rate of transcription of genetic information from DNA to messenger RNA, by binding to a specific DNA sequence. The function of a transcription factor is to regulate—turn on and off—genes in order to make sure they are expressed in the right cell at the right time and in the right amount throughout the life of the cell and the organism.

“Rel A” refers to a Transcription factor p65 also known as nuclear factor NF-kappa-B p65 subunit. It is a protein that in humans is encoded by the RELA gene. Rel A, also known as p65, is a Rel-associated protein involved in NF-κB heterodimer formation, nuclear translocation and activation. NF-κB is an essential transcription factor complex involved in all types of cellular processes, including cellular metabolism, chemotaxis, etc. Phosphorylation and acetylation of Rel A are crucial post-translational modifications required for NF-κB activation. Rel A has also been shown to modulate immune responses, and activation of Rel A is positively associated with multiple types of cancer.

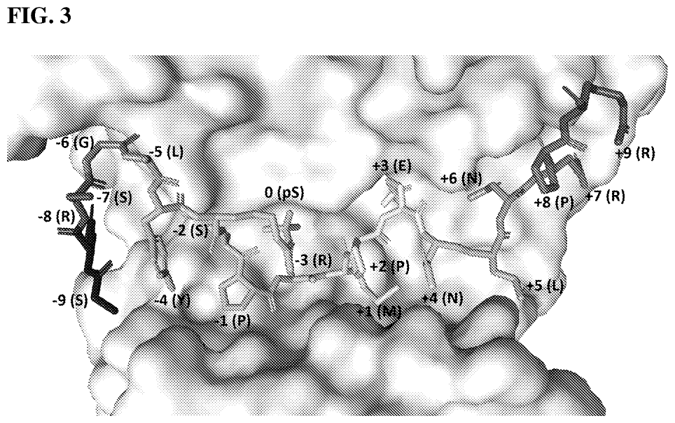

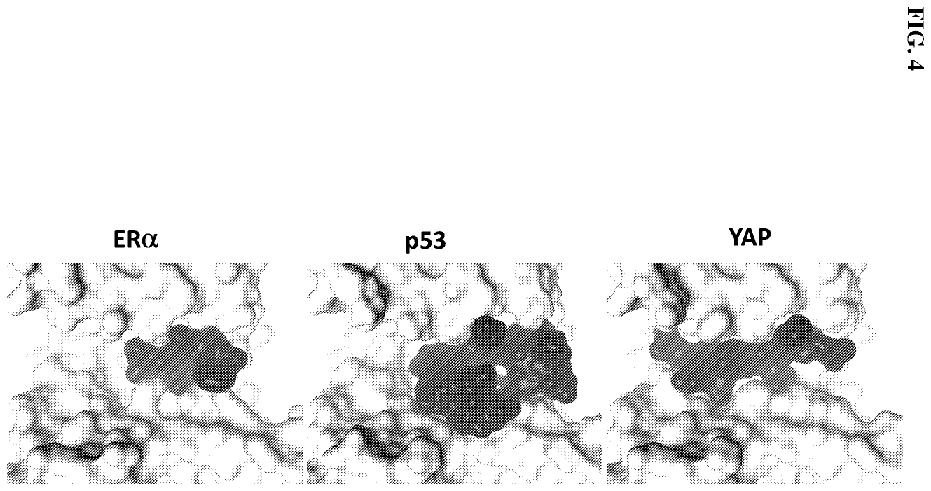

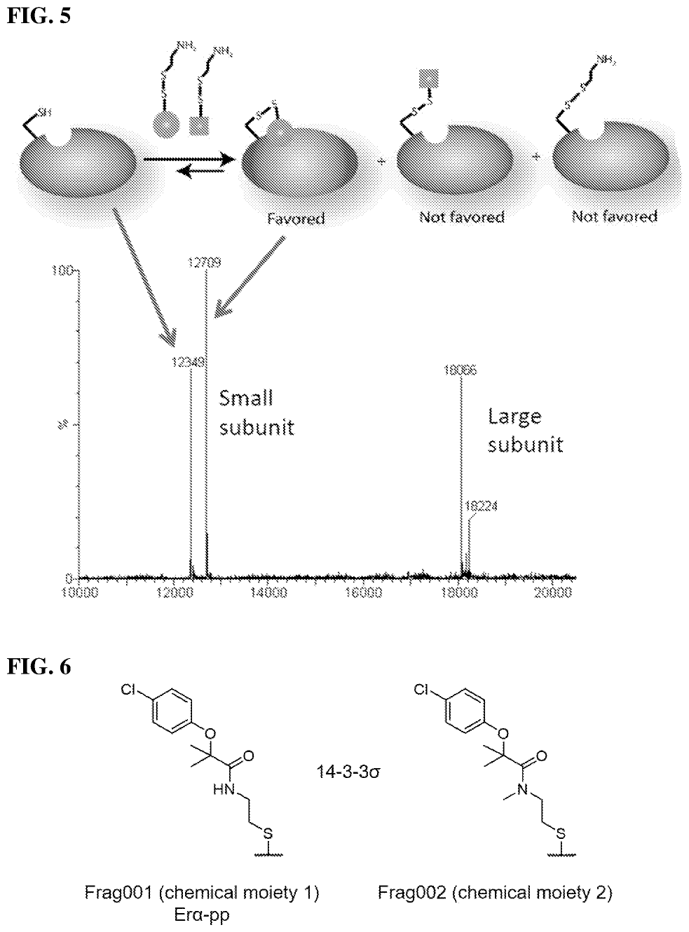

In embodiments, the solvent exposed reactive amino acid side chain of the 14-3-3σ client protein contacting the first candidate compound, is from residue −10 to +10 of the 14-3-3σ client protein, as numbered relative to the phosphorylated serine or threonine residues, shown in FIG. 3. The sequences vary from 14-3-3σ client protein to 14-3-3σ client protein. The structures of the bound phosphopeptides also vary significantly.

In embodiments, the 14-3-3σ client protein includes an amino acid mutation.

In embodiments, the 14-3-3σ client protein is selected from those listed in Table 1. In embodiments, the 14-3-3σ client protein is ERα, ERRγ, TASK3, ExoS, MYC, Rel A, FOXO-1 or TAZ or functional fragment thereof. In embodiments, the 14-3-3σ client protein is ERRγ or functional fragment thereof. In embodiments the client is a phosphorylated peptide (a phosphopeptide) derived from the 14-3-3σ client protein. In embodiments, the 14-3-3σ client is a phosphorylated peptide (phosphopeptide) representing the 14-3-3σ protein binding motif of the client protein. In embodiments, the 14-3-3 client protein is selected from those listed in Table 1. In embodiments, the 14-3-3 client protein is ERα, ERRγ, TASK3, ExoS, MYC, Rel A, FOXO-1 or TAZ or functional fragment thereof.

In embodiments, the conjugate-client complex is further contacted with a second candidate compound. In embodiments, the second candidate compound is soaked into conjugate-client complex co-crystal. In embodiments, the conjugate-client complex further includes a second candidate chemical moiety covalently bound to the first chemical moiety. In embodiments, the conjugate-client complex further includes a second candidate chemical moiety non-covalently bound to the first chemical moiety. In embodiments, the second candidate chemical moiety is in immediate contact with the first candidate chemical moiety. In embodiments, the second candidate chemical moiety is within about 10 A°, 5 A°, 4 A°, 3 A°, 2 A°, or 1 A°, of the first candidate chemical moiety. In embodiments, the second candidate chemical moiety is within about 10 A°, 5 A°, 4 A°, 3 A°, 2 A°, or 1 A°, of the first candidate chemical moiety, and connected through a linker. In embodiments, the second candidate chemical moiety is within about 10 A° of the first candidate chemical moiety. In embodiments, the second candidate chemical moiety is within about 5 A° of the first candidate chemical moiety. In embodiments, the second candidate chemical moiety is within about 4 A° of the first candidate chemical moiety. In embodiments, the second candidate chemical moiety is within about 3 A° of the first candidate chemical moiety. In embodiments, the second candidate chemical moiety is within about 2 A° of the first candidate chemical moiety. In embodiments, the second candidate chemical moiety is within about 1 A° of the first candidate chemical moiety. In embodiments, the second candidate chemical moiety is within about 10 A° of the first candidate chemical moiety, and connected through a linker. In embodiments, the second candidate chemical moiety is within about 5 A° of the first candidate chemical moiety, and connected through a linker. In embodiments, the second candidate chemical moiety is within about 4 A° of the first candidate chemical moiety, and connected through a linker. In embodiments, the second candidate chemical moiety is within about 3 A° of the first candidate chemical moiety, and connected through a linker. In embodiments, the second candidate chemical moiety is within about 2 A° of the first candidate chemical moiety, and connected through a linker. In embodiments, the second candidate chemical moiety is within about 1 A° of the first candidate chemical moiety, and connected through a linker. In embodiments, the binding of the second candidate chemical moiety is detected using NMR. “A” unit as used in this paragraph refers to Angstrom or Angstroms.

In embodiments, the conjugate-protein complex is further contacted with a second candidate compound. In embodiments, the second candidate compound is soaked into conjugate-protein complex co-crystal. In embodiments, the conjugate-protein complex further includes a second candidate chemical moiety covalently bound to the first chemical moiety. In embodiments, the conjugate-protein complex further includes a second candidate chemical moiety non-covalently bound to the first chemical moiety. In embodiments, the second candidate chemical moiety is in immediate contact with the first candidate chemical moiety. In embodiments, the second candidate chemical moiety is within about 10 A°, 5 A°, 4 A°, 3 A°, 2 A°, or 1 A°, of the first candidate chemical moiety. In embodiments, the second candidate chemical moiety is within about 10 A°, 5 A°, 4 A°, 3 A°, 2 A°, or 1 A°, of the first candidate chemical moiety, and connected through a linker. In embodiments, the second candidate chemical moiety is within about 10 A° of the first candidate chemical moiety. In embodiments, the second candidate chemical moiety is within about 5 A° of the first candidate chemical moiety. In embodiments, the second candidate chemical moiety is within about 4 A° of the first candidate chemical moiety. In embodiments, the second candidate chemical moiety is within about 3 A° of the first candidate chemical moiety. In embodiments, the second candidate chemical moiety is within about 2 A° of the first candidate chemical moiety. In embodiments, the second candidate chemical moiety is within about 1 A° of the first candidate chemical moiety. In embodiments, the second candidate chemical moiety is within about 10 A° of the first candidate chemical moiety, and connected through a linker. In embodiments, the second candidate chemical moiety is within about 5 A° of the first candidate chemical moiety, and connected through a linker. In embodiments, the second candidate chemical moiety is within about 4 A° of the first candidate chemical moiety, and connected through a linker. In embodiments, the second candidate chemical moiety is within about 3 A° of the first candidate chemical moiety, and connected through a linker. In embodiments, the second candidate chemical moiety is within about 2 A° of the first candidate chemical moiety, and connected through a linker. In embodiments, the second candidate chemical moiety is within about 1 A° of the first candidate chemical moiety, and connected through a linker. In embodiments, the binding of the second candidate chemical moiety is detected using NMR. “A” unit as used in this paragraph refers to Angstrom or Angstroms.

In embodiments, the first candidate chemical moiety is covalently bound to a solvent exposed reactive amino acid side chain of the 14-3-3 protein, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 protein, via disulfide bond, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 protein, via a bond that is not a disulfide bond, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a first solvent exposed reactive amino acid side chain of the protein, forming a conjugate client, then said conjugate client is further contacted with a second candidate moiety covalently bound to the client protein. In embodiments, the first candidate chemical moiety is covalently bound to a reactive amino acid side chain of the 14-3-3 protein, forming a conjugate client, then said conjugate client is further contacted with a second candidate moiety covalently bound to the 14-3-3 client protein. In embodiments, the first candidate chemical moiety is covalently bound to a reactive amino acid side chain of the 14-3-3 protein, via disulfide bond, forming a conjugate client, then said conjugate client is further contacted with a second candidate moiety covalently bound to the 14-3-3 client protein. In embodiments, the first candidate chemical moiety is covalently bound to a reactive amino acid side chain of the 14-3-3 protein, via disulfide bond, forming a conjugate client, then said conjugate client is further contacted with a second candidate moiety covalently bound to the 14-3-3 client protein, via disulfide bond.

In embodiments, the first candidate chemical moiety is covalently bound to a reactive amino acid side chain of the 14-3-3 protein, via a bond that is not a disulfide bond, forming a conjugate client, then said conjugate client is further contacted with a second candidate moiety covalently bound to the 14-3-3 client protein. In embodiments, the first candidate chemical moiety is covalently bound to a reactive amino acid side chain of the 14-3-3 protein, via a bond that is not a disulfide bond, forming a conjugate client, then said conjugate client is further contacted with a second candidate moiety covalently bound to the 14-3-3 client protein, via a bond that is not a disulfide bond.

In embodiments, the first candidate chemical moiety is covalently bound to a solvent exposed reactive amino acid side chain of the 14-3-3 protein, forming a protein conjugate, then said protein conjugate is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 protein, via disulfide bond, forming a protein conjugate, then said protein conjugate is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 protein, via a bond that is not a disulfide bond, forming a protein conjugate, then said protein conjugate is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a first solvent exposed reactive amino acid side chain of the protein, forming a protein conjugate, then said protein conjugate is further contacted with a second candidate moiety covalently bound to the client protein. In embodiments, the first candidate chemical moiety is covalently bound to a reactive amino acid side chain of the 14-3-3 protein, forming a protein conjugate, then said protein conjugate is further contacted with a second candidate moiety covalently bound to the 14-3-3 client protein. In embodiments, the first candidate chemical moiety is covalently bound to a reactive amino acid side chain of the 14-3-3 protein, via disulfide bond, forming a protein conjugate, then said protein conjugate is further contacted with a second candidate moiety covalently bound to the 14-3-3 client protein. In embodiments, the first candidate chemical moiety is covalently bound to a reactive amino acid side chain of the 14-3-3 protein, via disulfide bond, forming a protein conjugate, then said protein conjugate is further contacted with a second candidate moiety covalently bound to the 14-3-3 client protein, via disulfide bond.

In embodiments, the first candidate chemical moiety is covalently bound to a reactive amino acid side chain of the 14-3-3 protein, via a bond that is not a disulfide bond, forming a protein conjugate, then said protein conjugate is further contacted with a second candidate moiety covalently bound to the 14-3-3 client protein. In embodiments, the first candidate chemical moiety is covalently bound to a reactive amino acid side chain of the 14-3-3 protein, via a bond that is not a disulfide bond, forming a protein conjugate, then said protein conjugate is further contacted with a second candidate moiety covalently bound to the 14-3-3 client protein, via a bond that is not a disulfide bond.

In embodiments, the first candidate chemical moiety is covalently bound to a solvent exposed reactive amino acid side chain of the 14-3-3 client protein, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 client protein, via disulfide bond, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a solvent exposed reactive amino acid side chain of the 14-3-3 client protein, via a bond that is not a disulfide bond, forming a conjugate client, then said conjugate client is further contacted with a second candidate compound. In embodiments, the second candidate chemical moiety is covalently attached to the first candidate chemical moiety. In embodiments, the second candidate chemical moiety is non-covalently attached to the first candidate chemical moiety.

In embodiments, the first candidate chemical moiety is covalently bound to a first solvent exposed reactive amino acid side chain of the client protein, forming a conjugate client, then said conjugate client is further contacted with a second candidate moiety covalently bound to the protein. In embodiments, the first candidate chemical moiety is covalently bound to a reactive amino acid side chain of the 14-3-3 client protein, forming a conjugate client, then said conjugate client is further contacted with a second candidate moiety covalently bound to the 14-3-3 protein. In embodiments, the first candidate chemical moiety is covalently bound to a reactive amino acid side chain of the 14-3-3 client protein, via disulfide bond, forming a conjugate client, then said conjugate client is further contacted with a second candidate moiety covalently bound to the 14-3-3 protein. In embodiments, the first candidate chemical moiety is covalently bound to a reactive amino acid side chain of the 14-3-3 client protein, via disulfide bond, forming a conjugate client, then said conjugate client is further contacted with a second candidate moiety covalently bound to the 14-3-3 protein, via disulfide bond.

In embodiments, the first candidate chemical moiety is covalently bound to a reactive amino acid side chain of the 14-3-3 client protein, via a bond that is not a disulfide bond, forming a conjugate client, then said conjugate client is further contacted with a second candidate moiety covalently bound to the 14-3-3 protein. In embodiments, the first candidate chemical moiety is covalently bound to a reactive amino acid side chain of the 14-3-3 client protein, via a bond that is not a disulfide bond, forming a conjugate client, then said conjugate client is further contacted with a second candidate moiety covalently bound to the 14-3-3 protein, via a bond that is not a disulfide bond.

In embodiments, the protein client complex is contacted simultaneously with a first candidate compound and a second candidate compound. In embodiments, said first and second candidate compounds are non-covalently bound to the protein client complex, and to each other.

In embodiments, the protein client complex is contacted sequentially with a first candidate compound and then with a second candidate compound. In embodiments, the first candidate compound is non-covalently bound to the protein client complex and the second candidate compound is also non-covalently bound to the protein client complex and to the first candidate chemical moiety. In embodiments, the first and second candidate chemical moieties are covalently bound to each other, while being non-covalently bound to the protein client complex.

In embodiments, first candidate compound and second candidate compound are optimized such that no disulfide bonding to the protein and/or client protein is necessary for stabilization of the protein-protein interactions. In embodiments, first candidate compound and second candidate compound are optimized such that the first chemical moiety is covalently bound to the protein not via disulfide bond (e.g., via any other type of covalent bond), and the second compound is then contacted with the conjugate-client complex and is non-covalently bound. In embodiments, first candidate compound and second candidate compound are optimized such that the first chemical moiety is covalently bound to the protein not via disulfide bond (e.g., via any other type of covalent bond), and the second compound is then contacted with the conjugate-client complex and is covalently bound to the first chemical moiety. In embodiments, first candidate compound and second candidate compound are optimized such that the first chemical moiety is covalently bound to the protein not via disulfide bond (e.g., via any other type of covalent bond) and the second chemical moiety is covalently bound to the client protein not via disulfide bond (e.g., via any other type of covalent bond), and the two conjugates are then contacted with each other.

In an aspect, provided herein is a method for treating a disease in a subject in need thereof, the method including administering to the subject an effective amount of a chemical compound that stabilizes binding of a protein to a client protein, wherein the chemical compound is identified by any one of the methods described herein (including embodiments, examples, figures, or Tables).

In an aspect, provided herein is a compound described herein, that stabilizes binding of a protein to a client protein, for use in a method of treating a disease including administering to a subject an effective amount of the compound.

In an aspect, provided herein is use of a compound described herein, that stabilizes binding of a protein to a client protein, in the manufacture of a medicament for the treatment of a disease, the use including administering to a subject an effective amount of the compound.

In embodiments, the disease is a cancer, inflammatory disease, metabolic disease, neurodegenerative disease, or infection.

In embodiments, the disease is a cancer. In embodiments, the disease is an inflammatory disease. In embodiments, the disease is a metabolic disease. In embodiments, the disease is a neurodegenerative disease. In embodiments, the disease is an infection. In embodiments, the disease is an infectious disease.

In embodiments, the disease is an immune response related disease. In embodiments, the disease is an autoimmune disease.

In embodiments, the cancer is brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, or Non-Hodgkin's Lymphomas. In embodiments, the cancer is leukemias, lymphomas, carcinomas or sarcomas. In embodiments, the cancer is cancer of the thyroid, endocrine system, brain, breast, cervix, colon, head & neck, liver, kidney, lung, ovary, pancreas, rectum, stomach, or uterus.

In embodiments, the cancer is thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In embodiments, the neurodegenerative disease is Huntington Disease. In embodiments, the neurodegenerative disease is Alzheimer Disease. In embodiments, the neurodegenerative disease is Parkinson's Disease. In embodiments, the neurodegenerative disease is frontotemporal dementia. In embodiments, the method includes reducing protein aggregates (e.g., in the brain). In embodiments, the method includes reducing TDP-43 aggregates (e.g., in the brain). In embodiments, the neurodegenerative disease is amyotrophic lateral sclerosis. In embodiments, the neurodegenerative disease is chronic traumatic encephalopathy. In embodiments, the neurodegenerative disease is traumatic brain injury (e.g., concussion).

In embodiments, the metabolic disease is diabetes. In embodiments, the metabolic disease is type I diabetes. In embodiments, the metabolic disease is type II diabetes. In embodiments, the metabolic disease is obesity. In embodiments, the metabolic disease is metabolic syndrome. In embodiments, the metabolic disease is a mitochondrial disease (e.g., dysfunction of mitochondria or aberrant mitochondrial function).

In embodiments, the inflammatory disease is autoimmune disease, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis.

In embodiments, the immune response related disease is an autoimmune disease. In embodiments, the autoimmune disease is Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

In embodiments, the infection or infectious disease is viral, bacterial, fungal, protozoal, parasitic or prion disease. In embodiments, the infection or infectious disease is caused by a pathogenic bacteria. Pathogenic bacteria are bacteria which cause diseases (e.g., in humans). In embodiments, the infection or infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the infection or infectious disease is Bacterial vaginosis (i.e., bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e., a bacterial inflammation of the meninges); Bacterial pneumonia (i.e., a bacterial infection of the lungs); Urinary tract infection; Bacterial gastroenteritis; or Bacterial skin infections (e.g., impetigo, or cellulitis). In embodiments, the infection or infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

In an aspect is provided a method of identifying a chemical compound that stabilizes binding of a protein to a client protein, the method including: contacting a first candidate compound with a protein including a solvent exposed reactive amino acid side chain proximal to a client protein binding site, thereby forming a protein conjugate, wherein the first candidate compound includes a first candidate chemical moiety covalently bound to a first reactive group, wherein the first reactive group is specifically reactive with the solvent exposed reactive amino acid side chain, which is not a cysteine side chain; contacting the protein conjugate with the client protein thereby forming a conjugate-client complex; and detecting an increased stability of the conjugate-client complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that stabilizes binding of the protein to the client protein.

In an aspect is provided a method of identifying a chemical compound that stabilizes binding of a protein to a client protein, the method including: contacting a client protein with a protein including a solvent exposed reactive amino acid side chain proximal to a client protein binding site, thereby forming a protein-client complex; contacting the protein-client complex with a first candidate compound thereby forming a conjugate-client complex, wherein the first candidate compound includes a first candidate chemical moiety covalently bound to a first reactive group, wherein the first reactive group is specifically reactive with the solvent exposed reactive amino acid side chain, which is not a cysteine side chain, and wherein the first candidate compound covalently attaches to the solvent exposed reactive amino acid side chain to form the conjugate-client complex; and detecting an increased stability of the conjugate-client complex relative to the stability of the protein-client complex, wherein the protein-client complex includes the client protein and the protein in the absence of the first candidate compound covalently bound to the solvent exposed reactive amino acid side chain, thereby identifying the first candidate compound as the first chemical compound that stabilizes binding of the protein to the client protein.

In embodiments, the protein is a 14-3-3 protein. In embodiments, the solvent exposed reactive amino acid side chain of the 14-3-3 protein, proximal to the 14-3-3 client protein binding site, is the side chain of a methionine, tryptophan, tyrosine, lysine or histidine. In embodiments, the 14-3-3 protein includes an amino acid mutation. In embodiments, the 14-3-3 client protein is ERα, ERRγ, TASK3, ExoS, MYC, Rel A, FOXO-1 or TAZ. In embodiments, the 14-3-3 client protein is ERα. In embodiments, the conjugate-client complex further includes a second candidate compound covalently bound to the first chemical compound. In embodiments, the conjugate-client complex is further contacted with a second candidate compound, such that the conjugate-client complex is non-covalently attached to the second candidate compound.

In an aspect is provided a method of identifying a chemical compound that stabilizes binding of a protein to a client protein, the method including: contacting a first candidate compound with a client protein including a solvent exposed reactive amino acid side chain, thereby forming a client protein conjugate, wherein the first candidate compound includes a first candidate chemical moiety covalently bound to a first reactive group, wherein the first reactive group is specifically reactive with the solvent exposed reactive amino acid side chain; contacting the client protein conjugate with a protein thereby forming a conjugate-protein complex; and detecting an increased stability of the conjugate-protein complex relative to the stability of a protein-client complex, wherein the protein-client complex includes the client protein and the protein in the absence of the first candidate compound covalently bound to the solvent exposed reactive amino acid side chain, thereby identifying the first candidate compound as the first chemical compound that stabilizes binding of the protein to the client protein.

In an aspect is provided a method of identifying a chemical compound that stabilizes binding of a protein to a client protein, the method including: contacting a protein with a client protein including a solvent exposed reactive amino acid side chain thereby forming a protein-client complex; contacting the protein-client complex with a first candidate compound thereby forming a conjugate-protein complex, wherein the first candidate compound includes a first candidate chemical moiety covalently bound to a first reactive group, wherein the first reactive group is specifically reactive with the solvent exposed reactive amino acid side chain, and wherein the first candidate compound covalently attaches to the solvent exposed reactive amino acid side chain to form the conjugate-protein complex; and detecting an increased stability of the conjugate-protein complex relative to the stability of the protein-client complex, wherein the protein-client complex includes the protein and the client protein in the absence of the first candidate compound covalently bound to the solvent exposed reactive amino acid side chain, thereby identifying the first candidate compound as the first chemical compound that stabilizes binding of the protein to the client protein.

In embodiments, the protein is a 14-3-3 protein. In embodiments, the solvent exposed reactive amino acid side chain of the 14-3-3 client protein is the side chain of a cysteine, methionine, tryptophan, tyrosine, lysine or histidine. In embodiments, the solvent exposed reactive amino acid side chain of the 14-3-3 client protein is the side chain of a cysteine. In embodiments, the solvent exposed reactive amino acid side chain of the 14-3-3 client protein includes a thiol. In embodiments, the 14-3-3 client protein includes an amino acid mutation. In embodiments, the 14-3-3 client protein is ERα, ERRγ, TASK3, ExoS, MYC, Rel A, FOXO-1 or TAZ. In embodiments, the 14-3-3 client protein is ERRγ. In embodiments, the conjugate-protein complex further includes a second candidate compound covalently bound to the first chemical compound. In embodiments, the conjugate-protein complex is further contacted with a second candidate compound, such that the conjugate-protein complex is non-covalently attached to the second candidate compound.

In an aspect is provided a method of treating a disease in a subject in need thereof, the method including administering to the subject an effective amount of a chemical compound that stabilizes binding of a protein to a client protein, wherein the chemical compound is identified by any one of the methods described herein.

In embodiments, the disease is cancer, inflammatory disease, metabolic disease, neurodegenerative disease, or infection.

In one aspect, is provided a method of making a chemical compound that modulates (e.g., stabilizes) binding of a protein (e.g., 14-3-3 protein) to a client protein, the method including: 1. identifying a first chemical compound that modulates (e.g., stabilizes) binding of a protein (e.g., 14-3-3 protein) to a client protein, including the steps: (a) contacting a first candidate compound with a protein including a first reactive amino acid side chain proximal to a client protein binding site, thereby forming a first protein conjugate, wherein said first candidate compound includes a first candidate chemical moiety covalently bound to a first reactive group, wherein said first reactive group is specifically reactive with said first reactive amino acid side chain; (b) contacting said first protein conjugate with said client protein thereby forming a first conjugate-client complex; and (c) detecting a modulated (e.g., an increased) stability of said first conjugate-client complex relative to the stability of a protein-client complex, wherein said protein-client complex includes said client protein and said protein in the absence of said first candidate compound covalently bound to said first reactive group, thereby identifying a first candidate compound that modulates (e.g., stabilizes) binding of said protein to said client protein; 2. Optionally identifying a second chemical compound that modulates (e.g., stabilizes) binding of the protein (e.g., 14-3-3 protein) to the client protein, including the steps: (a) contacting a second candidate compound with the protein including a second reactive amino acid side chain proximal to a client protein binding site, thereby forming a second protein conjugate, wherein said second candidate compound includes a second candidate chemical moiety covalently bound to a second reactive group, wherein said second reactive group is specifically reactive with said second reactive amino acid side chain, wherein said second reactive amino acid side chain proximal to a client protein binding site is different from the first reactive amino acid side chain proximal to a client protein binding site; (b) contacting said second protein conjugate with said client protein thereby forming a second conjugate-client complex; and (c) detecting a modulated (e.g., an increased) stability of said second conjugate-client complex relative to the stability of a protein-client complex, wherein said protein-client complex includes said client protein and said protein in the absence of said second candidate compound covalently bound to said second reactive group, thereby identifying said second candidate compound that modulates (e.g., stabilizes) binding of said protein to said client protein; 3. optionally repeating step 2 above; 4. Optionally identifying a third chemical compound that modulates (e.g., stabilizes) binding of the protein (e.g., 14-3-3 protein) to the client protein, including the steps: (a) contacting a third candidate compound with the client protein including a third reactive amino acid side chain proximal to a protein binding site, thereby forming a third client protein conjugate, wherein said third candidate compound includes a third candidate chemical moiety covalently bound to a third reactive group, wherein said third reactive group is specifically reactive with said third reactive amino acid side chain; (b) contacting said third client protein conjugate with said protein thereby forming a third conjugate-protein complex; and (c) detecting a modulated (e.g., an increased) stability of said third conjugate-protein complex relative to the stability of a protein-client complex, wherein said protein-client complex includes said client protein and said protein in the absence of said third candidate compound covalently bound to said third reactive group, thereby identifying said third candidate compound that modulates (e.g., stabilizes) binding of said protein to said client protein; 5. optionally repeating step 4 above; and 6. making the chemical compound that modulates (e.g., stabilizes) binding of a protein to a client protein wherein the chemical compound includes: (a) the first candidate chemical moiety identified in step 1; (b) the one or more second candidate chemical moieties identified in steps 2 and 3; (c) the one or more third candidate chemical moieties identified in steps 4 and 5; and (d) covalent linkers connecting the moieties recited in steps 6(a) to 6(c) above.

In one aspect, is provided a method of making a chemical compound that modulates (e.g., stabilizes) binding of a protein (e.g., 14-3-3 protein) to a client protein, the method including: 1. identifying a first chemical compound that modulates (e.g., stabilizes) binding of a protein (e.g., 14-3-3 protein) to a client protein, including the steps: (a) contacting a first candidate compound with a client protein including a first reactive amino acid side chain proximal to a protein binding site, thereby forming a first client protein conjugate, wherein said first candidate compound includes a first candidate chemical moiety covalently bound to a first reactive group, wherein said first reactive group is specifically reactive with said first reactive amino acid side chain; (b) contacting said first client protein conjugate with said protein thereby forming a first conjugate-protein complex; and (c) detecting a modulated (e.g, an increased) stability of said first conjugate-protein complex relative to the stability of a protein-client complex, wherein said protein-client complex includes said client protein and said protein in the absence of said first candidate compound covalently bound to said first reactive group, thereby identifying a first candidate compound that modulates (e.g., stabilizes) binding of said protein to said client protein; 2. Optionally identifying a second chemical compound that modulates (e.g., stabilizes) binding of the protein (e.g., 14-3-3 protein) to the client protein, including the steps: (a) contacting a second candidate compound with the protein including a second reactive amino acid side chain proximal to a client protein binding site, thereby forming a second protein conjugate, wherein said second candidate compound includes a second candidate chemical moiety covalently bound to a second reactive group, wherein said second reactive group is specifically reactive with said second reactive amino acid side chain, wherein said second reactive amino acid side chain proximal to a client protein binding site is different from the first reactive amino acid side chain proximal to a client protein binding site; (b) contacting said second protein conjugate with said client protein thereby forming a second conjugate-client complex; and (c) detecting a modulated (e.g., an increased) stability of said second conjugate-client complex relative to the stability of a protein-client complex, wherein said protein-client complex includes said client protein and said protein in the absence of said second candidate compound covalently bound to said second reactive group, thereby identifying said second candidate compound that modulates (e.g., stabilizes) binding of said protein to said client protein; 3. optionally repeating step 2 above; 4. Optionally identifying a third chemical compound that modulates (e.g., stabilizes) binding of the protein (e.g., 14-3-3 protein) to the client protein, including the steps: (a) contacting a third candidate compound with the client protein including a third reactive amino acid side chain proximal to a protein binding site, thereby forming a third client protein conjugate, wherein said third candidate compound includes a third candidate chemical moiety covalently bound to a third reactive group, wherein said third reactive group is specifically reactive with said third reactive amino acid side chain; (b) contacting said third client protein conjugate with said protein thereby forming a third conjugate-protein complex; and (c) detecting a modulated (e.g., an increased) stability of said third conjugate-protein complex relative to the stability of a protein-client complex, wherein said protein-client complex includes said client protein and said protein in the absence of said third candidate compound covalently bound to said third reactive group, thereby identifying said third candidate compound that modulates (e.g., stabilizes) binding of said protein to said client protein; 5. optionally repeating step 4 above; and 6. making the chemical compound that modulates (e.g., stabilizes) binding of a protein to a client protein wherein the chemical compound includes: (a) the first candidate chemical moiety identified in step 1; (b) the one or more second candidate chemical moieties identified in steps 2 and 3; (c) the one or more third candidate chemical moieties identified in steps 4 and 5; and (d) covalent linkers connecting the moieties recited in steps 6(a) to 6(c) above.

In embodiments, the first reactive amino acid side chain is a cysteine side chain. In embodiments, the first reactive amino acid side chain is a lysine side chain. In embodiments, the first reactive amino acid side chain is a histidine side chain. In embodiments, the first reactive amino acid side chain is a methionine side chain. In embodiments, the first reactive amino acid side chain is a tyrosine side chain. In embodiments, the first reactive amino acid side chain is a tryptophan side chain. In embodiments, the first reactive amino acid side chain is a cysteine side chain of the amino acid corresponding to C38 of 14-3-3σ. In embodiments, the first reactive amino acid side chain is not a cysteine side chain. In embodiments, the first reactive amino acid side chain is a lysine side chain of the amino acid corresponding to K122 of 14-3-3σ.

In embodiments, the second reactive amino acid side chain is a cysteine side chain. In embodiments, the second reactive amino acid side chain is a lysine side chain. In embodiments, the second reactive amino acid side chain is a histidine side chain. In embodiments, the second reactive amino acid side chain is a methionine side chain. In embodiments, the second reactive amino acid side chain is a tyrosine side chain. In embodiments, the second reactive amino acid side chain is a tryptophan side chain. In embodiments, the second reactive amino acid side chain is a cysteine side chain of the amino acid corresponding to C38 of 14-3-3σ. In embodiments, the second reactive amino acid side chain is not a cysteine side chain. In embodiments, the second reactive amino acid side chain is a lysine side chain of the amino acid corresponding to K122 of 14-3-3σ.

In embodiments, the third reactive amino acid side chain is a cysteine side chain. In embodiments, the third reactive amino acid side chain is a lysine side chain. In embodiments, the third reactive amino acid side chain is a histidine side chain. In embodiments, the third reactive amino acid side chain is a methionine side chain. In embodiments, the third reactive amino acid side chain is a tyrosine side chain. In embodiments, the third reactive amino acid side chain is a tryptophan side chain. In embodiments, the third reactive amino acid side chain is a cysteine side chain of the amino acid corresponding to C38 of 14-3-3σ. In embodiments, the third reactive amino acid side chain is not a cysteine side chain. In embodiments, the third reactive amino acid side chain is a lysine side chain of the amino acid corresponding to K122 of 14-3-3σ.

### VII. Embodiments

Embodiment P1. A compound having the general formula:

R¹-L¹-W-L³-R³.

wherein
L¹ and L³ are independently substituted or unsubstituted covalent linkers;
R¹ is a 14-3-3 K120 binding moiety;
W is a substituted or unsubstituted 14-3-3 binding linker; and
R³ is a client protein binding moiety.

Embodiment P2. The compound of embodiment P1, wherein R¹ is a 14-3-3 K120 covalent binding moiety.

Embodiment P3. The compound of embodiment P1, wherein R¹ is a 14-3-3 K120 non-covalent binding moiety.

Embodiment P4. The compound of one of embodiments P1 to P3, further comprising R², wherein R² is a 14-3-3 C38 non-covalent binding moiety or a 14-3-3 C38 covalent binding moiety.

Embodiment P5. The compound of embodiment P4, wherein R² is a 14-3-3 C38 non-covalent binding moiety.

Embodiment P6. The compound of embodiment P4, wherein R² is a 14-3-3 C38 covalent binding moiety.

Embodiment P7. A compound having the general formula:

R²-L²-W-L³-R³.

wherein
L² and L³ are independently substituted or unsubstituted covalent linkers;
R² is a 14-3-3 C38 non-covalent binding moiety;
W is a substituted or unsubstituted 14-3-3 binding linker; and
R³ is a client protein binding moiety.

Embodiment P8. The compound of embodiment P7, comprising R¹, wherein R¹ is a 14-3-3 K120 binding moiety.

Embodiment P9. The compound of embodiment P8, wherein R¹ is a 14-3-3 K120 covalent binding moiety.

Embodiment P10. The compound of embodiment P8, wherein R¹ is a 14-3-3 K120 non-covalent binding moiety.

Embodiment P11. The compound of one of embodiments P1 to P10, wherein W is substituted with -L⁵-R⁵, wherein

L⁵ is a substituted or unsubstituted covalent linker;
R⁵ is a 14-3-3 D215 binding moiety.

Embodiment P12. The compound of one of embodiments P1 to P11, wherein W is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P13. The compound of one of embodiments P1 to P6 and P8 to P12, wherein

R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is independently an integer from 0 to 4; and
m1 and v1 are independently 1 or 2.

Embodiment P14. The compound of one of embodiments P1 to P6 and P8 to P12, wherein

R¹ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment P15. The compound of one of embodiments P1, P4 to P6, P8, and P11 to P12, wherein

### R¹ is

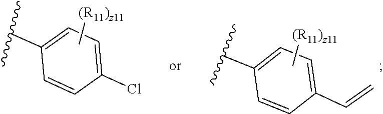

R¹¹ is independently halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹¹ substituents may optionally be joined to form a substituted or unsubstituted cycloalkyl, substituted or unsubstituted heterocycloalkyl, substituted or unsubstituted aryl, or substituted or unsubstituted heteroaryl;
and z11 is an integer from 0 to 4.

Embodiment P16. The compound of one of embodiments P1, P4 to P6, P8, and P11 to P12, wherein

### R¹ is

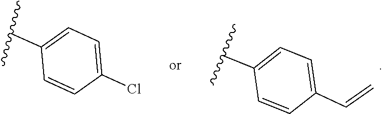

Embodiment P17. The compound of one of embodiments P4 to P16, wherein

R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is independently an integer from 0 to 4; and
m2 and v2 are independently 1 or 2.

Embodiment P18. The compound of one of embodiments P4 to P16, wherein

R² is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment P19. The compound of one of embodiments P4, P6, and P11 to P16, wherein

R² is -L^{2A}-L^{2B}-E2;

L^{2A} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{2B} is independently a bond, —NH—, —C(O)NH—, —NHC(O)NH—, substituted or unsubstituted heteroalkylene, substituted or unsubstituted heterocycloalkylene, or substituted or unsubstituted heteroarylene;

### E2 is —SH, —SSR²⁶,

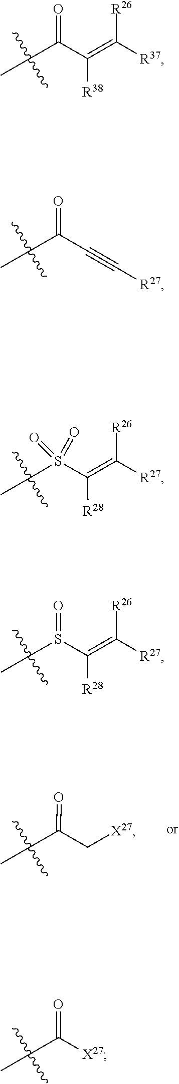

R²⁶, R²⁷, and R²⁸ is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X²⁷ is independently —F, —Cl, —Br, or —I.

Embodiment P20. The compound of one of embodiments P4, P6, and P11 to P16, wherein

R² is -L^{2A}-L^{2B}-E2;
L^{2A} is independently a bond;
L^{2B} is independently —NH—;

### E2 is

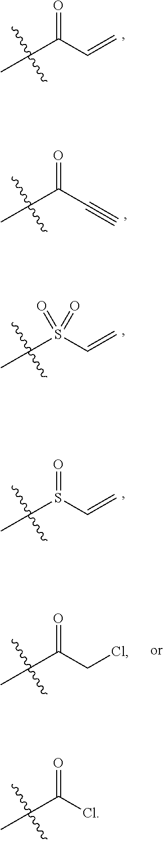

Embodiment P21. The compound of one of embodiments P11 to P20, wherein R⁵ is independently hydrogen, halogen, —CX⁵₃, —CHX⁵₂, —CH₂X⁵, —OCX⁵₃, —OCH₂X⁵, —OCHX⁵₂, —CN, —SOᵥ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X⁵ is independently —F, —Cl, —Br, or —I;
n5 is independently an integer from 0 to 4; and
m5 and v5 are independently 1 or 2.

Embodiment P22. The compound of one of embodiments P11 to P20, wherein

R⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P23. The compound of one of embodiments P1 to P22, wherein R³ is independently hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{3A}, R^{3B}, R^{3C}, and R^{3D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X³ is independently —F, —Cl, —Br, or —I;
n3 is independently an integer from 0 to 4; and
m3 and v3 are independently 1 or 2.

Embodiment P24. The compound of one of embodiments P1 to P22, wherein

R³ is -L^{3A}-L^{3B}-E3;
L^{3A} is independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is independently a bond, —NH—, —C(O)NH—, —NHC(O)NH—, substituted or unsubstituted heteroalkylene, substituted or unsubstituted heterocycloalkylene, or substituted or unsubstituted heteroarylene;

### E3 is —SH,

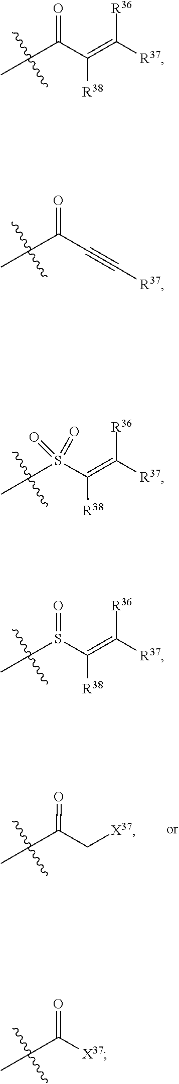

R³⁶, R³⁷, and R³⁸ is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X³⁷ is independently —F, —Cl, —Br, or —I.

Embodiment P25. The compound of one of embodiments P1 to P22, wherein

R³ is -L^{3A}-L^{3B}-E3;
L^{3A} is independently a bond;
L^{3B} is independently —NH—;

### E3 is

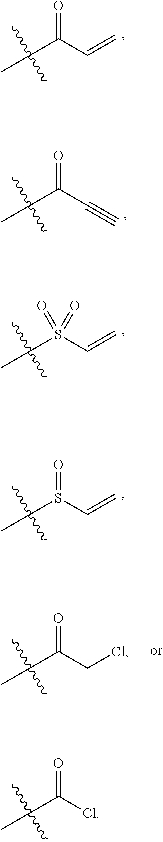

Embodiment P26. A pharmaceutical composition comprising the compound of any one of embodiments P1 to P25, or a pharmaceutically acceptable salt thereof, and a pharmaceutically acceptable excipient.

Embodiment P27. A method of increasing the level of a 14-3-3 protein-client protein complex in a subject, said method comprising administering a compound of one of embodiments P1 to P25 to said subject.

Embodiment P28. A method of embodiment P27, wherein the client protein of the 14-3-3 protein-client protein complex is an estrogen receptor.

Embodiment P29. A method of embodiment P27, wherein the client protein of the 14-3-3 protein-client protein complex is an estrogen related receptor gamma.

Embodiment P30. A method of embodiment P27, wherein the client protein of the 14-3-3 protein-client protein complex is p65.

Embodiment P31. A method of increasing the level of a 14-3-3 protein-client protein complex in a cell, said method comprising contacting the cell with a compound of one of embodiments P1 to P25.

Embodiment P32. A method of treating an inflammatory disease, cancer, an autoimmune disease, a neurodegenerative disease, a metabolic disease, or cystic fibrosis in a subject in need thereof, said method comprising administering to the subject in need thereof an effective amount of a compound of one of embodiments P1 to P25.

Embodiment P33. A method of treating a cancer in a subject in need thereof, said method comprising administering to the subject in need thereof an effective amount of a compound of one of embodiments P1 to P25.

Embodiment P34. The method of embodiment P33, wherein the cancer is breast cancer.

Embodiment P35. The method of one of embodiments P33 to P34, further comprising co-administering an anti-cancer agent to said subject in need.

Embodiment P36. A method of identifying a chemical compound that modulates the binding of a protein to a client protein, the method comprising:

contacting a first candidate compound with a protein comprising a solvent exposed reactive amino acid side chain proximal to a client protein binding site, thereby forming a protein conjugate, wherein said first candidate compound comprises a first candidate chemical moiety covalently bound to a first reactive group, wherein said first reactive group is specifically reactive with said solvent exposed reactive amino acid side chain, which is not a cysteine side chain;
contacting said protein conjugate with said client protein thereby forming a conjugate-client complex; and
detecting a change in stability of said conjugate-client complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment P37. The method of embodiment P36, wherein the method identifies a chemical compound that stabilizes the binding of a protein to a client protein comprising detecting an increase in stability of said conjugate-client complex relative to the stability of a protein-client complex.

Embodiment P38. A method of identifying a chemical compound that modulates binding of a protein to a client protein, the method comprising:

contacting a client protein with a protein comprising a solvent exposed reactive amino acid side chain proximal to a client protein binding site, thereby forming a protein-client complex;
contacting said protein-client complex with a first candidate compound thereby forming a conjugate-client complex, wherein said first candidate compound comprises a first candidate chemical moiety covalently bound to a first reactive group, wherein said first reactive group is specifically reactive with said solvent exposed reactive amino acid side chain, which is not a cysteine side chain, and wherein said first candidate compound covalently attaches to said solvent exposed reactive amino acid side chain to form said conjugate-client complex; and
detecting a change in stability of said conjugate-client complex relative to the stability of said protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment P39. The method of embodiment P38, wherein the method identifies a chemical compound that stabilizes the binding of a protein to a client protein comprising detecting an increase in stability of said conjugate-client complex relative to the stability of a protein-client complex.

Embodiment P40. The method of one of embodiments P36 to P39, wherein the protein is a 14-3-3 protein.

Embodiment P41. The method of any one of embodiments P36 to P40, wherein the solvent exposed reactive amino acid side chain of the 14-3-3 protein, proximal to the 14-3-3 client protein binding site, is the side chain of a methionine, tryptophan, tyrosine, lysine or histidine.

Embodiment P42. The method of any one of embodiments P40 to P41, wherein the 14-3-3 protein comprises an amino acid mutation.

Embodiment P43. The method of any one of embodiments P40 to P42, wherein the 14-3-3 client protein is ERα, ERRγ, TASK3, ExoS, MYC, Rel A, FOXO-1 or TAZ.

Embodiment P44. The method of embodiment P43, wherein the 14-3-3 client protein is ERα.

Embodiment P45. The method of any one of embodiments P36 to P44, wherein the conjugate-client complex further comprises a second candidate compound covalently bound to said first chemical compound.

Embodiment P46. The method of any one of embodiments P36 to P44, wherein the conjugate-client complex is further contacted with a second candidate compound, such that the conjugate-client complex is non-covalently attached to said second candidate compound.

Embodiment P47. A method of identifying a chemical compound that modulates binding of a protein to a client protein, the method comprising:

contacting a first candidate compound with a client protein comprising a solvent exposed reactive amino acid side chain, thereby forming a client protein conjugate, wherein said first candidate compound comprises a first candidate chemical moiety covalently bound to a first reactive group, wherein said first reactive group is specifically reactive with said solvent exposed reactive amino acid side chain;
contacting said client protein conjugate with a protein thereby forming a conjugate-protein complex; and
detecting a change in stability of said conjugate-protein complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment P48. A method of identifying a chemical compound that modulates binding of a protein to a client protein, the method comprising:

contacting a protein with a client protein comprising a solvent exposed reactive amino acid side chain thereby forming a protein-client complex;
contacting said protein-client complex with a first candidate compound thereby forming a conjugate-protein complex, wherein said first candidate compound comprises a first candidate chemical moiety covalently bound to a first reactive group, wherein said first reactive group is specifically reactive with said solvent exposed reactive amino acid side chain, and wherein said first candidate compound covalently attaches to said solvent exposed reactive amino acid side chain to form said conjugate-protein complex; and
detecting a change in stability of said conjugate-protein complex relative to the stability of said protein-client complex, wherein said protein-client complex comprises said protein and said client protein in the absence of said first candidate compound covalently bound to said solvent exposed reactive amino acid side chain, thereby identifying said first candidate compound as the first chemical compound that modulates binding of said protein to said client protein.

Embodiment P49. The method of one of embodiments P47 to P48, wherein the method identifies a chemical compound that stabilizes the binding of a protein to a client protein comprising detecting an increase in stability of said conjugate-protein complex relative to the stability of a protein-client complex.

Embodiment P50. The method of one of embodiments P47 to P49, wherein the protein is a 14-3-3 protein.

Embodiment P51. The method of any one of embodiments P47 to P50, wherein the solvent exposed reactive amino acid side chain of the 14-3-3 client protein is the side chain of a cysteine, methionine, tryptophan, tyrosine, lysine or histidine.

Embodiment P52. The method of embodiment P51, wherein the solvent exposed reactive amino acid side chain of the 14-3-3 client protein is the side chain of a cysteine.

Embodiment P53. The method of embodiment P52, wherein the solvent exposed reactive amino acid side chain of the 14-3-3 client protein comprises a thiol.

Embodiment P54. The method of any one of embodiments P50 to P53, wherein the 14-3-3 client protein comprises an amino acid mutation.

Embodiment P55. The method of any one of embodiments P50 to P54, wherein the 14-3-3 client protein is ERα, ERRγ, TASK3, ExoS, MYC, Rel A, FOXO-1 or TAZ.

Embodiment P56. The method of embodiment P55, wherein the 14-3-3 client protein is ERRγ.

Embodiment P57. The method of any one of embodiments P47 to P56, wherein the conjugate-protein complex further comprises a second candidate compound covalently bound to said first chemical compound.

Embodiment P58. The method of any one of embodiments P47 to P56, wherein the conjugate-protein complex is further contacted with a second candidate compound, such that the conjugate-protein complex is non-covalently attached to said second candidate compound.

Embodiment P59. A method of treating a disease in a subject in need thereof, the method comprising administering to the subject an effective amount of a chemical compound that stabilizes binding of a protein to a client protein, wherein the chemical compound is identified by any one of the methods of embodiments P36 to P58.

Embodiment P60. The method of embodiment P59, wherein the disease is cancer, inflammatory disease, metabolic disease, neurodegenerative disease, or infection.

### VIII. Additional Embodiments

Embodiment 1. A method of identifying a chemical compound that modulates the binding of a protein to a client protein, the method comprising:

contacting a first candidate compound with a protein comprising a solvent exposed reactive amino acid side chain proximal to a client protein binding site, thereby forming a protein conjugate, wherein said first candidate compound comprises a first candidate chemical moiety covalently bound to a first reactive group, wherein said first reactive group is specifically reactive with said solvent exposed reactive amino acid side chain, which is not a cysteine side chain;
contacting said protein conjugate with said client protein thereby forming a conjugate-client complex; and
detecting a change in stability of said conjugate-client complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment 2. The method of embodiment 1, wherein the method identifies a chemical compound that stabilizes the binding of a protein to a client protein comprising detecting an increase in stability of said conjugate-client complex relative to the stability of a protein-client complex.

Embodiment 3. The method of embodiment 1 or 2, wherein the protein is a 14-3-3 protein.

Embodiment 4. The method of embodiment 3, wherein the solvent exposed reactive amino acid side chain of the 14-3-3 protein, proximal to the 14-3-3 client protein binding site, is the side chain of a methionine, tryptophan, tyrosine, lysine or histidine.

Embodiment 5. The method of embodiment 3 or 4, wherein the 14-3-3 protein comprises an amino acid mutation.

Embodiment 6. The method of any one of embodiments 3-5, wherein the 14-3-3 client protein is ERα, ERRγ, TASK3, ExoS, MYC, Rel A, FOXO-1, p65, or TAZ.

Embodiment 7. The method of any one of embodiments 1-6, wherein the conjugate-client complex further comprises a second candidate compound covalently bound to said first candidate compound.

Embodiment 8. The method of any one of embodiments 1-6, wherein the conjugate-client complex is further contacted with a second candidate compound, such that the conjugate-client complex is non-covalently attached to said second candidate compound.

Embodiment 9. A method of identifying a chemical compound that modulates binding of a protein to a client protein, the method comprising:

contacting a client protein with a protein comprising a solvent exposed reactive amino acid side chain proximal to a client protein binding site, thereby forming a protein-client complex;
contacting said protein-client complex with a first candidate compound thereby forming a conjugate-client complex, wherein said first candidate compound comprises a first candidate chemical moiety covalently bound to a first reactive group, wherein said first reactive group is specifically reactive with said solvent exposed reactive amino acid side chain, which is not a cysteine side chain, and wherein said first candidate compound covalently attaches to said solvent exposed reactive amino acid side chain to form said conjugate-client complex; and
detecting a change in stability of said conjugate-client complex relative to the stability of said protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment 10. The method of embodiment 9, wherein the method identifies a chemical compound that stabilizes the binding of a protein to a client protein comprising detecting an increase in stability of said conjugate-client complex relative to the stability of a protein-client complex.

Embodiment 11. The method of embodiment 9 or 10, wherein the protein is a 14-3-3 protein.

Embodiment 12. The method of embodiment 11, wherein the solvent exposed reactive amino acid side chain of the 14-3-3 protein, proximal to the 14-3-3 client protein binding site, is the side chain of a methionine, tryptophan, tyrosine, lysine or histidine.

Embodiment 13. The method of embodiment 11 or 12, wherein the 14-3-3 protein comprises an amino acid mutation.

Embodiment 14. The method of any one of embodiments 11-13, wherein the 14-3-3 client protein is ERα, ERRγ, TASK3, ExoS, MYC, Rel A, FOXO-1, p65, or TAZ.

Embodiment 15. The method of any one of embodiments 9-14, wherein the conjugate-client complex further comprises a second candidate compound covalently bound to said first candidate compound.

Embodiment 16. The method of any one of embodiments 9-14, wherein the conjugate-client complex is further contacted with a second candidate compound, such that the conjugate-client complex is non-covalently attached to said second candidate compound.

Embodiment 17. A method of identifying a chemical compound that modulates binding of a protein to a client protein, the method comprising:

contacting a first candidate compound with a client protein comprising a solvent exposed reactive amino acid side chain, thereby forming a client protein conjugate, wherein said first candidate compound comprises a first candidate chemical moiety covalently bound to a first reactive group, wherein said first reactive group is specifically reactive with said solvent exposed reactive amino acid side chain;
contacting said client protein conjugate with a protein thereby forming a conjugate-protein complex; and
detecting a change in stability of said conjugate-protein complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

Embodiment 18. The method of embodiment 17, wherein the method identifies a chemical compound that stabilizes the binding of a protein to a client protein comprising detecting an increase in stability of said conjugate-protein complex relative to the stability of a protein-client complex.

Embodiment 19. The method of embodiment 17 or 18, wherein the protein is a 14-3-3 protein.

Embodiment 20. The method of embodiment 19, wherein the solvent exposed reactive amino acid side chain of the 14-3-3 client protein is the side chain of a cysteine, methionine, tryptophan, tyrosine, lysine or histidine.

Embodiment 21. The method of embodiment 20, wherein the solvent exposed reactive amino acid side chain of the 14-3-3 client protein is the side chain of a cysteine.

Embodiment 22. The method of embodiment 21, wherein the solvent exposed reactive amino acid side chain of the 14-3-3 client protein comprises a thiol.

Embodiment 23. The method of any one of embodiments 19-22, wherein the 14-3-3 client protein comprises an amino acid mutation.

Embodiment 24. The method of any one of embodiments 19-23, wherein the 14-3-3 client protein is ERα, ERRγ, TASK3, ExoS, MYC, Rel A, FOXO-1, p65, or TAZ.

Embodiment 25. The method of any one of embodiments 17-24, wherein the conjugate-protein complex further comprises a second candidate compound covalently bound to said first candidate compound.

Embodiment 26. The method of any one of embodiments 17-24, wherein the conjugate-protein complex is further contacted with a second candidate compound, such that the conjugate-protein complex is non-covalently attached to said second candidate compound.

Embodiment 27. A method of identifying a chemical compound that modulates binding of a protein to a client protein, the method comprising:

contacting a protein with a client protein comprising a solvent exposed reactive amino acid side chain thereby forming a protein-client complex;
contacting said protein-client complex with a first candidate compound thereby forming a conjugate-protein complex, wherein said first candidate compound comprises a first candidate chemical moiety covalently bound to a first reactive group, wherein said first reactive group is specifically reactive with said solvent exposed reactive amino acid side chain, and wherein said first candidate compound covalently attaches to said solvent exposed reactive amino acid side chain to form said conjugate-protein complex; and
detecting a change in stability of said conjugate-protein complex relative to the stability of said protein-client complex, wherein said protein-client complex comprises said protein and said client protein in the absence of said first candidate compound covalently bound to said solvent exposed reactive amino acid side chain, thereby identifying said first candidate compound as the first chemical compound that modulates binding of said protein to said client protein.

Embodiment 28. The method of embodiment 27, wherein the method identifies a chemical compound that stabilizes the binding of a protein to a client protein comprising detecting an increase in stability of said conjugate-protein complex relative to the stability of a protein-client complex.

Embodiment 29. The method of embodiment 27 or 28, wherein the protein is a 14-3-3 protein.

Embodiment 30. The method of embodiment 29, wherein the solvent exposed reactive amino acid side chain of the 14-3-3 client protein is the side chain of a cysteine, methionine, tryptophan, tyrosine, lysine or histidine.

Embodiment 31. The method of embodiment 30, wherein the solvent exposed reactive amino acid side chain of the 14-3-3 client protein is the side chain of a cysteine.

Embodiment 32. The method of embodiment 31, wherein the solvent exposed reactive amino acid side chain of the 14-3-3 client protein comprises a thiol.

Embodiment 33. The method of any one of embodiments 29-32, wherein the 14-3-3 client protein comprises an amino acid mutation.

Embodiment 34. The method of any one of embodiments 29-33, wherein the 14-3-3 client protein is ERα, ERRγ, TASK3, ExoS, MYC, Rel A, FOXO-1, p65, or TAZ.

Embodiment 35. The method of any one of embodiments 27-34, wherein the conjugate-protein complex further comprises a second candidate compound covalently bound to said first candidate compound.

Embodiment 36. The method of any one of embodiments 27-34, wherein the conjugate-protein complex is further contacted with a second candidate compound, such that the conjugate-protein complex is non-covalently attached to said second candidate compound.

Embodiment 37. A method of treating a disease in a subject in need thereof, the method comprising administering to the subject an effective amount of a chemical compound that stabilizes binding of a protein to a client protein, wherein the chemical compound is identified by a method of one of embodiments 1 to 36.

Embodiment 38. The method of embodiment 37, wherein the disease is cancer, inflammatory disease, metabolic disease, neurodegenerative disease, or infection.

Embodiment 39. A compound having the general formula:

R¹-L¹-W-L³-R³,

wherein:
L¹ and L³ are independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;

### X¹ is —F, —Cl, —Br, or —I;

n1 is an integer from 0 to 4;
m1 and v1 are independently 1 or 2;
W is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is -L^{3A}-L^{3B}-E³, hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

### X³ is —F, —Cl, —Br, or —I;

n3 is an integer from 0 to 4;
m3 and v3 are independently 1 or 2;
L^{3A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —NH—, —C(O)NH—, —NHC(O)NH—, substituted or unsubstituted heteroalkylene, substituted or unsubstituted heterocycloalkylene, or substituted or unsubstituted heteroarylene;

### E3 is —SH,

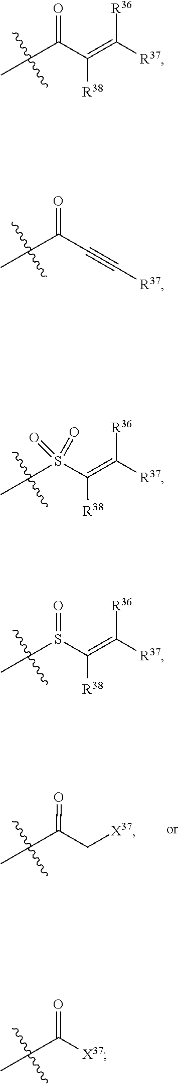

R³⁶, R³⁷, and R³⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

### X³⁷ is —F, —Cl, —Br, or —I.

Embodiment 40. The compound of embodiment 39, wherein

R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment 41. The compound of embodiment 39, wherein

### R¹ is

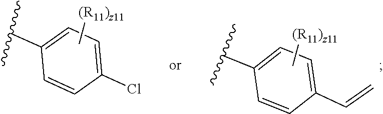

R¹¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹¹ substituents may optionally be joined to form a substituted or unsubstituted cycloalkyl, substituted or unsubstituted heterocycloalkyl, substituted or unsubstituted aryl, or substituted or unsubstituted heteroaryl; and
z11 is an integer from 0 to 4.

Embodiment 42. The compound of embodiment 39, wherein

### R¹ is

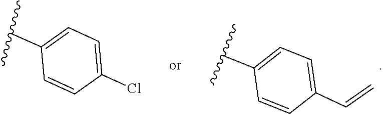

Embodiment 43. The compound of any one of embodiments 39-42, further comprising R², wherein R² is a 14-3-3 C38 binding moiety.

Embodiment 44. The compound of embodiment 43, wherein R² is a 14-3-3 C38 non-covalent binding moiety.

Embodiment 45. The compound of embodiment 43, wherein R² is a 14-3-3 C38 covalent binding moiety.

Embodiment 46. The compound of embodiment 43, wherein

R² is hydrogen, halogen, —CX²₃, —CHX²₂, —CH₂X², —OCX²₃, —OCH₂X², —OCHX²₂, —CN, —SOₙ₂R_{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N3, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl;

### X² is —F, —Cl, —Br, or —I;

n2 is an integer from 0 to 4; and
m2 and v2 are independently 1 or 2.

Embodiment 47. The compound of embodiment 43, wherein

R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N3, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment 48. The compound of embodiment 43, wherein

R² is -L^{2A}-L^{2B}-E2;
L^{2A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{2B} is a bond, —NH—, —C(O)NH—, —NHC(O)NH—, substituted or unsubstituted heteroalkylene, substituted or unsubstituted heterocycloalkylene, or substituted or unsubstituted heteroarylene;

### E2 is —SH, —SSR²⁶,

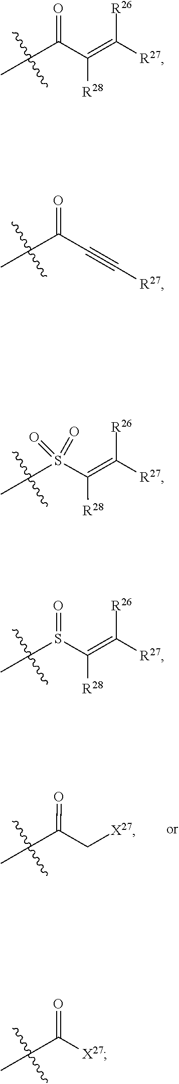

R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

### X²⁷ is —F, —Cl, —Br, or —I.

Embodiment 49. The compound of embodiment 43, wherein

R² is -L^{2A}-L^{2B}-E2;
L^{2A} is a bond;

### L^{2B} is —NH—; and

### E2 is

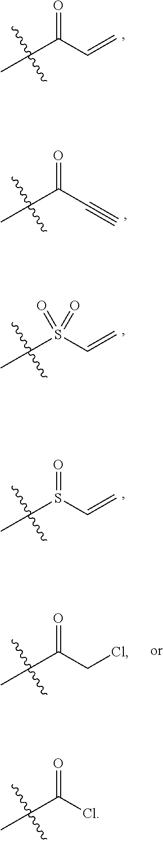

Embodiment 50. The compound of any one of embodiments 39-49, wherein W is substituted with -L⁵-R⁵, wherein

L⁵ is a substituted or unsubstituted covalent linker; and
R⁵ is a 14-3-3 D215 binding moiety.

Embodiment 51. The compound of embodiment 50, wherein

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

### X⁵ is —F, —Cl, —Br, or —I;

n5 is an integer from 0 to 4; and
m5 and v5 are independently 1 or 2.

Embodiment 52. The compound of embodiment 50, wherein

R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 53. The compound of any one of embodiments 39-52, wherein

R³ is -L^{3A}-L^{3B}-E3, wherein
L^{3A} is a bond;

### L^{3B} is —NH—; and

### E3 is

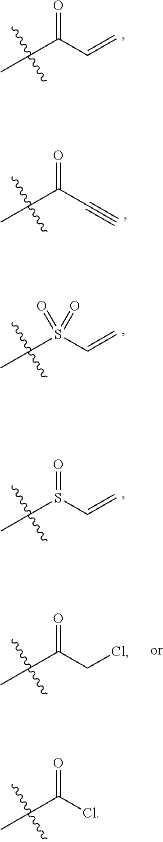

Embodiment 54. A compound having the general formula:

R²-L²-W-L³-R³;

wherein:
L² and L³ are independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein:
R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;

### X² is —F, —Cl, —Br, or —I;

n2 is an integer from 0 to 4;
m2 and v2 are independently 1 or 2;
W is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is -L^{3A}-L^{3B}-E3, hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3A}, R^{3B}, R^{3C}, and R^{3D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

### X³ is —F, —Cl, —Br, or —I;

n3 is an integer from 0 to 4;
m3 and v3 are independently 1 or 2;
L^{3A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —NH—, —C(O)NH—, —NHC(O)NH—, substituted or unsubstituted heteroalkylene, substituted or unsubstituted heterocycloalkylene, or substituted or unsubstituted heteroarylene;

### E3 is —SH,

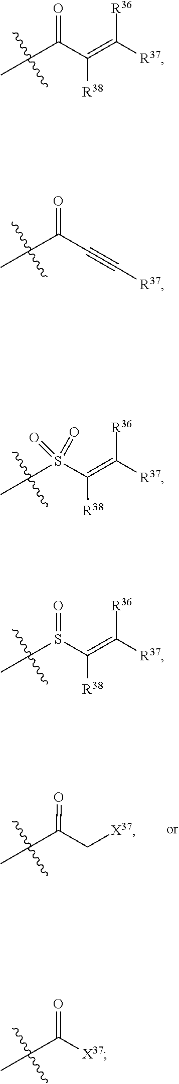

R³⁶, R³⁷, and R³⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

### X³⁷ is —F, —Cl, —Br, or —I.

Embodiment 55. The compound of embodiment 54, further comprising R¹, wherein R¹ is a 14-3-3 K120 binding moiety.

Embodiment 56. The compound of embodiment 55, wherein R¹ is a 14-3-3 K120 covalent binding moiety.

Embodiment 57. The compound of embodiment 55, wherein R¹ is a 14-3-3 K120 non-covalent binding moiety.

Embodiment 58. The compound of embodiment 55, wherein

R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} substituents bonded to the same nitrogen atom may optionally be joined to form a substituted or unsubstituted heterocycloalkyl or substituted or unsubstituted heteroaryl;

### X¹ is —F, —Cl, —Br, or —I;

n1 is an integer from 0 to 4; and
m1 and v1 are independently 1 or 2.

Embodiment 59. The compound of embodiment 55, wherein

R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment 60. The compound of embodiment 55, wherein

### R¹ is

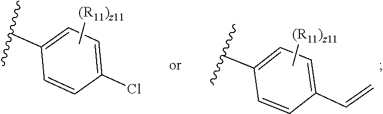

R¹¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹¹ substituents may optionally be joined to form a substituted or unsubstituted cycloalkyl, substituted or unsubstituted heterocycloalkyl, substituted or unsubstituted aryl, or substituted or unsubstituted heteroaryl; and
z11 is an integer from 0 to 4.

Embodiment 61. The compound of embodiment 55, wherein

### R¹ is

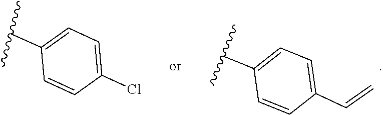

Embodiment 62. The compound of any one of embodiments 54-61, wherein

R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment 63. The compound of any one of embodiments 54-62, wherein W is substituted with -L⁵-R⁵, wherein

L⁵ is a substituted or unsubstituted covalent linker; and
R⁵ is a 14-3-3 D215 binding moiety.

Embodiment 64. The compound of embodiment 63, wherein

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

### X⁵ is —F, —Cl, —Br, or —I;

n5 is an integer from 0 to 4; and
m5 and v5 are independently 1 or 2.

Embodiment 65. The compound of embodiment 63, wherein

R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 66. The compound of any one of embodiments 54-65, wherein

R³ is -L^{3A}-L^{3B}-E3;
L^{3A} is a bond;

### L^{3B} is —NH—; and

### E3 is

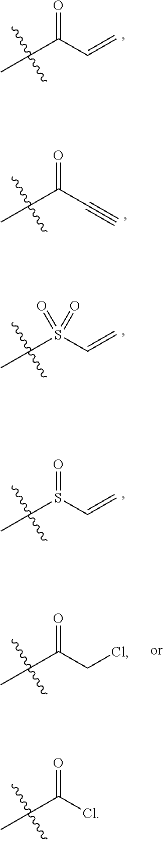

Embodiment 67. A pharmaceutical composition comprising the compound of any one of embodiments 39 to 66, or a pharmaceutically acceptable salt thereof, and a pharmaceutically acceptable excipient.

Embodiment 68. A method of increasing the level of a 14-3-3 protein-client protein complex in a subject, said method comprising administering a compound of one of embodiments 39 to 66 to said subject.

Embodiment 69. The method of embodiment 68, wherein the client protein of the 14-3-3 protein-client protein complex is an estrogen receptor.

Embodiment 70. The method of embodiment 68, wherein the client protein of the 14-3-3 protein-client protein complex is TAZ.

Embodiment 71. The method of embodiment 68, wherein the client protein of the 14-3-3 protein-client protein complex is p65.

Embodiment 72. A method of increasing the level of a 14-3-3 protein-client protein complex in a cell, said method comprising contacting the cell with a compound of one of embodiments 39 to 66.

Embodiment 73. A method of treating an inflammatory disease, cancer, an autoimmune disease, a neurodegenerative disease, a metabolic disease, or cystic fibrosis in a subject in need thereof, said method comprising administering to the subject in need thereof an effective amount of a compound of one of embodiments 39 to 66.

Embodiment 74. A method of treating a cancer in a subject in need thereof, said method comprising administering to the subject in need thereof an effective amount of a compound of one of embodiments 39 to 66.

Embodiment 75. The method of embodiment 74, wherein the cancer is breast cancer.

Embodiment 76. The method of embodiment 74, further comprising co-administering an anti-cancer agent to said subject in need.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Compound Screening

We envisioned that disulfide trapping (tethering) would be a promising technology to develop such a platform. Disulfide trapping allows site-directed selection of ligands and readily measures cooperative binding—qualities that address the main challenges posed by screening for PPI stabilizers. Since the technology was pioneered by Wells, Erlanson and co-workers (475), disulfide trapping has successfully identified allele-specific inhibitors of oncogenic KRas (G12C) (476), allosteric ligands of kinase PDK1 (477) and inhibitors of the IL-2/IL-2receptor PPI (478-479). Here, we offer the first demonstration of the tethering technology to identify small-molecule stabilizers of a protein complex.

We selected the interaction between the hub protein 14-3-3 and the phosphorylated motif derived from the breast-cancer-associated transcription factor Estrogen Receptor a (ERα) as a suitable and relevant test case. With >300 cellular interaction partners, including Raf kinases (480), heat shock proteins, (481) oncogenes (482) and tumor suppressors (p53) (483), 14-3-3 proteins are central regulators in many biological processes and pathologies. (484-486) For example, 14-3-3 binding antagonizes multiple transcription factors that act as oncogenic drivers. Since inhibition of transcriptional activity is a central therapeutic challenge in cancer, we have focused our efforts towards identifying small molecule stabilizers for this PPI class. De Vries-van Leeuwen et al reported that ERα is phosphorylated at the penultimate residue T594 and that binding of this site to 14-3-3 reduces its estradiol-dependent transcriptional activity. Inhibition of ERα activity is enhanced by the natural product Fusicoccin A (FC-A), which binds at the 14-3-3/ERα interface. (482) Stabilizing this PPI was proposed to be a valid alternative strategy for interfering with ERα-positive breast cancer.

Here, we identified several disulfide fragments that each bound cooperatively to a complex of 14-3-3 and ERα-derived phosphopeptide (ERα-pp). Hits selectively increased the binding affinity between ERα-pp and 14-3-3 by as much as 40-fold; multiple x-ray co-structures suggested the mechanism of stabilization. Disulfide tethering is a promising approach to identify starting points to specifically stabilize protein-peptide interactions and provides a first and long-needed, systematic screening platform for PPI-stabilizing molecules.

Tethering uses a cysteine on the target protein as a reactivity handle to trap disulfide-containing fragments that have an inherent (weak) binding affinity for a target pocket near the cysteine. The bound fragments can then be detected by intact protein mass spectrometry (MS). (475, 487) Our disulfide trapping approach was designed to target FC-A's hydrophobic pocket at the 14-3-3σ/ERα interface. Sigma is the only one of seven human 14-3-3 isoforms that contains a native surface-exposed cysteine (C38) at the edge of this pocket. We further designed two protein constructs in which the wildtype cysteine was mutated (C38N; N being the most common residue at this position) and a cysteine introduced at positions 42 or 45, one or two α-helix turns towards the ERα binding site, respectively. The three 14-3-3σ cysteine constructs were screened both in apo form and in complex with a 15-mer phosphopeptide representing the 14-3-3-binding motif of ERα (ERα-pp; KYYITGEAEGFPApT⁵⁹⁴V (SEQ ID NO:2)). Phosphorylated motifs derived from 14-3-3 client proteins recapitulate key interactions of the PPI, and mutating the single phosphorylation site can completely abrogate the interaction in vitro and in cells. (488) Short 14-3-3 client-derived phosphopeptides can thus be used in vitro as surrogates for the PPI; this approach has been used to characterize FC-A/14-3-3/client complexes and to screen for inhibitors (489), e.g., of the 14-3-3/Tau PPI. (490-491)

Apo-14-3-3σ or the 14-3-3σ/ERα-pp complex was screened against a 1600-member disulfide library under mildly reducing conditions (100 μM betamercaptoethanol; βME). Conjugate formation for each individual reaction was analyzed by intact protein MS. Three peaks observed in mass spectra corresponded to apo, βME-capped, and fragment-conjugated 14-3-3σ. The ‘percent tethering’, defined as the intensity of the fragment-specific conjugate protein peak divided by the sum of the intensities for all protein peaks, was calculated for each individual experiment using an automated pipeline. (492)

For each screen, hits were categorized as competitive (only a hit in the apo screen), cooperative (preferentially a hit for the protein-peptide complex), or neutral (a hit both in the apo and the 14-3-3σ/ERα-pp complex). C38 yielded the highest fraction of cooperative hits, but the maximal percent tethering was low (<55% conjugated), suggesting that fragments bound to C38 had a low affinity for the pocket. Conversely, C45, closest to the target pocket, yielded a large fraction of hits with >75% conjugation, with more competitive than cooperative hits. Satisfyingly, C42, with an intermediate position, yielded hits for both apo and ERα-pp bound 14-3-3σ, suggesting an optimal distance and orientation towards the target pocket to identify potent and cooperative fragments.

For C42, the most cooperative fragment was 1; tethering increased 2.3-fold, from 26% (apo) to 60% (protein-peptide complex). A nearly identical compound, 2, was identified in both screens, with 46% tethering to the apo 14-3-3σ and 59% tethering to the complex. Compounds 1, 2, and seven additional fragments that bound to the 14-3-3σ(C42)/ERα-pp complex were selected for follow-up experiments.

The 14-3-3σ(C42)-binding hits were confirmed in dose-response experiments detected by intact protein MS. Both 1 and 2 demonstrated strong preferential binding to the 14-3-3σ/ERα-pp complex over the 14-3-3σ protein alone. Binding of the tethered fragment 2 was improved ˜300-fold, from an effective concentration (EC₅₀) ˜1 mM for apo to EC₅₀=3 μM for the ERα-pp bound 14-3-3σ. Fragment 1, the N-methylated version of 2, showed an EC₅₀˜100 μM for binding to apo but remained >80% tethered to 14-3-3σ bound to ERα-pp down to 100 nM fragment, even in the stringent disulfide-reducing condition of 1 mM βME. Cooperative binding was less pronounced for the other primary screening hits (data not shown)

The effect of 1 and 2 on the binding affinity between ERα-pp and 14-3-3σ(C42) was studied in fluorescence anisotropy experiments. 14-3-3σ(C42) was titrated into fluorescein-labeled ERα-pp in the presence of DMSO or saturating concentrations of fragments. The apparent dissociation constant of 14-3-3σ(C42)/ERα-pp (K_{d,app}) was 1.3 μM for the DMSO control, and decreased to 32 nM in the presence of 1, 92 nM in the presence of fragment 2, and 4.2 nM in the presence of the positive control FC-A. Thus, 1 and 2 stabilized the 14-3-36/ERα-pp complex by 40- and 14-fold, respectively.

We observed the same trend when we titrated the fragments into a mixture of 1 μM 14-3-3σ(C42) and 100 nM fluorescein-ERα-pp, conditions under which half of the peptide was initially bound. Fragments binding to the 14-3-3σ(C42)/ERα-pp complex increased the anisotropy, and hence the bound fraction, of fluorescein-ERα-pp. Additionally, from these experiments we observed that the kinetics of disulfide formation (i.e. stabilizing effect on ERα-pp binding) was dependent on the disulfide-fragment concentration, as evidenced by an increase in anisotropy values over time for 0.1-10 μM. The maximum effect was instantaneous at a saturating concentration (100 μM). Compound 1 displayed slightly more cooperative behavior, as reflected in a lower EC₅₀ (87 nM) compared to 2 (EC₅₀=209 nM). Notably, both were slightly more potent compared to FC-A (EC₅₀=216 nM).

Whereas this cooperative behavior for 1 was expected based on the initial criteria for hit selection, 2 was only slightly cooperative in the primary screen. To evaluate whether the single-concentration screen was reproducible, we further evaluated ten additional fragment hits, including three selected as ‘competitive’ from the screen. While moderate cooperativity was observed for most of the ‘cooperative’ fragments, the competitive and neutral fragments generally had no effect on ERα-pp binding, except for one ‘neutral’ fragment that modestly inhibited peptide binding. Thus, while single-concentration screening yielded reproducible cooperative fragments, screening at multiple doses could be advantageous.

To elucidate the molecular mechanism for cooperativity, fragments were soaked into co-crystals of 14-3-3σ an 8-mer ERα-pp. In addition to 1 and 2, electron density was resolved for three other C42 hits. Fragments 1-5 each contained an aromatic ring pointed into the back of the 14-3-3 pocket, oriented to make hydrophobic contact with the C-terminal V595 of ERα-pp. The chlorophenyl substitutions in 1, 2, and 3 were fully buried in the pocket. Whereas for 1, 2, and 4 continuous electron density could be traced from the bound cysteine, 3 and 5 had less complete density, perhaps suggesting disorder in their linker region. Interestingly, while all fragments had a phenyl ring in an analogous location, 3-5 showed significantly less cooperativity compared to 1 and 2. These data could suggest that the electronic nature of the ring and/or the stability of the ring orientation are critical for productive interactions with both 14-3-3σ and ERα-pp.

Comparison of hits from screening C42 and C45 revealed 6, which differed from 2 only in linker length (propyl vs ethyl, respectively) between the fragment and the disulfide-forming thiol. We solved the structure of 6 conjugated to 14-3-3σ(C45) in complex with ERα-pp and found electron density for the expected tethered fragment and parts of the linker. An overlay of 6-C45 with 2-C42 showed that the chloride moiety was positioned in the same pocket of 14-3-3, but the chlorophenyl ring was tilted so that the edge, rather than the face, of the phenyl ring was pointed towards ERα-pp V595. Indeed, 6 displayed low cooperativity when bound to 14-3-3σ(C45). Interestingly, 6 bound to 14-3-3σ(C42) showed similar cooperativity and binding affinity compared to 2 (EC₅₀ value of <1 μM in the presence of ERα-pp). The convergent positioning of the C42- and C45-targeted analogs 2 and 6 suggested that the fragments were selected based on their compatibility with the pocket formed by 14-3-3 and ERα-pp; however, the conformational restriction imposed by the anchoring residue determined how productively the fragment interacted with ERα-pp.

Together, the data for disulfide hits 1, 2 and 6 supported the hypothesis that the binding affinity of the fragments to the protein-peptide complex was driven by non-covalent interactions, which was further enhanced by the linker. To confirm, we tested a non-covalent analogue for binding to 14-3-3/ERα-pp by ligand-observed NMR in Tip and waterLOGSY experiments. Tip relaxation was significantly enhanced in the presence of 14-3-3/ERα-pp and a positive wateLOGSY signal was seen in the presence, but not in the absence, of 14-3-3/ERα-pp. These data demonstrated that the fragment bound to the complex even in the absence of a covalent linkage.

Finally, to investigate the selectivity of disulfide fragments for 14-3-3/ERα-pp, we selected the binding motifs of ExoS and TAZ as representative alternative 14-3-3 clients. (493-494) The TAZ phosphopeptide (TAZ-pp) extends through the druggable pocket, thereby restricting the space for fragment binding. ExoS is one of the few reported non-phosphorylated clients of 14-3-3, and also occupies almost the full length of the amphipathic groove, including the target pocket. We also included TASK3, which contains a C-terminal phosphoSV nearly identical to the phosphoTV motif in ERα. (495) In dose-response analysis by MS, a shift to the left was observed for the binding curve of 2 in the presence of TASK3 phosphopeptide (TASK3-pp) compared to apo 14-3-3σ(C42), indicating a similar ability for 2 to bind cooperatively to the 14-3-3σ(C42)/TASK3-pp and ERα-pp complexes, with EC₅₀ values of 7 μM and 3 μM, respectively. By contrast, for ExoS or TAZ-pp, the dose-response curve for binding of 2 was shifted to the right. Even at 500 μM, 2 just reached ˜40% bound, compared to 80% bound to apo 14-3-3σ(C42), and 100% bound to 14-3-3σ(C42)/ERα-pp or TASK3-pp. The effect of 2 on the binding affinity of 14-3-3σ(C42) for the different peptide partners was further quantified by fluorescence anisotropy, where 2 was titrated into a solution of fluorescently labeled TASK3-pp, ExoS, or TAZ-pp and 14-3-3σ(C42) at a concentration that allowed 20% binding of the peptide initially (Anisotropy (r) of ˜40 mAU). FC-A and DMSO were included as controls. In alignment with MS data, 2 increased the affinity between 14-3-3 and TASK3-pp (EC50=2 μM) and showed a destabilizing effect on ExoS and TAZ-pp binding (IC50=1.4 μM and 2 μM, respectively). Interestingly, the maximal anisotropy value for TASK3-pp was lower when 2 was titrated compared to FC-A; this difference was not observed when 2 and FC-A were titrated to ERα-pp. It might indicate a reduced stabilization of the distal regions of TASK3-pp. Furthermore, there was a 10-20 fold shift in the EC₅₀ for the stabilizing vs inhibiting effect of 2 on the binding of ERα-pp (100-200 nM) compared to TASK3-pp, ExoS and TAZ-pp (1-2 μM), indicating already partial selectivity for the hit fragment that can be further exploited by chemical optimization.

Small-molecule PPI stabilization has diverse therapeutic applications, justifying the pursuit of novel drug discovery strategies. The major and unmet challenge in this field is the lack of starting points for small-molecule stabilizer development. In contrast to conventional screening techniques, we find disulfide trapping to be highly suitable for early stabilizer discovery, likely because the technology is site-directed and the disulfide bond allows the fragment to fully saturate the binding site. We have validated the disulfide screening paradigm by selecting fragments that enhance the interaction between 14-3-3σ and an ERα-derived phosphopeptide (ERα-pp) and crystallized these fragments to learn the molecular requirements to achieve stabilization.

Disulfide-bound fragments bind cooperatively with ERα-pp to 14-3-3σ(C42), providing as much as a 40-fold increase in affinity for the 14-3-3σ(C42)/ERα-pp complex. Both the binding affinity and the degree of PPI stabilization depend on the chemical structure of the fragments and their orientation in the binding site. In particular, stabilization of the 14-3-3/ERα-pp complex correlated with the presence of para-chlorophenol ring oriented with its face towards the terminal valine of the peptide. Taken together, the biochemical data and crystal structures support the hypothesis that binding of the fragments was driven by the non-covalent interactions with the protein-peptide complex, which was confirmed in ligand-observed NMR experiments for a non-covalent analogue.

Towards development of the platform, we compared differential hits from three cysteine constructs of 14-3-3 and observed that the appropriate stringency of screening is essential for selecting fragments can engage the targeted pocket. In addition to the differences in the degree of tethering, we also observed that different types of hits (i.e., cooperative, neutral, competitive towards peptide binding) were more likely at different positions. Whereas we initially were very stringent towards selecting cooperative hits for follow-up, we found that ‘neutral’ hits displaying high intrinsic affinity for protein could also induce a cooperative effect when studied in more detail. Hence, future efforts could include multiple-dose screening to maximize the window between binding to 14-3-3 and to 14-3-3/peptide complexes.

One challenge when targeting PPI of proteins with many binding partners, such as 14-3-3, is client selectivity. Opportunities for selectivity result from the significant variation in phosphoprotein sequences and 14-3-3-binding modes, giving rise to differences in the protein-protein interface that small molecules could exploit. As demonstration, 2 shows the strongest cooperativity towards ERα-pp, secondly towards the ERα-like-peptide TASK3-pp, and at higher concentrations also partially influences the structurally unrelated 14-3-3/TAZ-pp or ExoS protein-peptide complexes. These differences could be further exploited by optimizing contacts with ERα-pp and tuning the ratio of intrinsic binding of the fragments to apo-14-3-3 versus binding to the 14-3-3/peptide complex.

It is important to note that 14-3-3 client proteins are much larger than the peptides studied here. However, 14-3-3 proteins exert their regulatory role specifically via phosphorylation-induced PPIs, and the phosphate group on a binding partner is usually the primary driver of the binding affinity. Therefore, even in the context of differential secondary interactions, a stabilizing effect on this primary interaction site will result in an overall increased stability of the full-length protein complex. To fully validate the utility of our fragments will require chemical optimization and characterization of the PPI in a biological environment. The principle innovation of these studies is the systematic platform for discovery of PPI stabilizing fragments that are then suitable for tried-and-true strategies to optimize fragments into chemical probes and/or drug leads.

The disulfide trapping strategy can be generalized to any 14-3-3/client pair. In addition to ERα, several other important transcription factors, including TAZ, Myc, RelA, and FOXO-1, are clients of 14-3-3, and this approach could conceivably develop modulators of multiple transcription factors. Furthermore, small molecules might be able to induce unnatural 14-3-3/protein complexes, allowing the exploration of synthetic biology. As a site-directed binding methodology, disulfide trapping is an ideal technology for such a platform approach. Systematic discovery of novel PPI stabilizers has the potential to access ‘undruggable’ targets and provide opportunities for intervention in previously inaccessible pathways.

The 14-3-3/ER stabilizer FC (Fusicoccin) inhibits estradiol dependent signaling. FC (a natural product) inhibits dimerization of E2/ER and tamoxifen/ER. FC also blocks DNA binding and transcription (482) (PNAS May 28, 2013 110 (22) 8894-8899). Disulfide trapping for stabilizers of 14-3-3σ/ERα-pp (15-mer phosphopeptide representing the 14-3-3σ binding motif of ERα), was measured (J. Am. Chem. Soc. 2019, 141, 8, 3524-3531, FIG. 1b). Different cysteine residues yield different 14-3-3/client stabilizing chemical moieties. At each tethering residue, bound chemical moieties can be competitive with peptide, noncompetitive, or cooperative (J. Am. Chem. Soc. 2019, 141, 8, 3524-3531, FIG. 1c). LC/MS spectra of tethering screen results for chemical moiety 1 conjugated to 14-3-3σ (C42) apo or ERα-pp bound, resulted in 26% and 60% tethering, respectively. 14-3-3σ (C42) expected mass: 26509 Da, βME capped mass: 26585 Da, protein-disulfide conjugate mass: 26795 Da. LC/MS dose response curves showing percentage of protein conjugate formation for titrations of disulfides to 14-3-3σ (C42) apo and bound to ERα-pp, were measured. Dose-response curve demonstrates tighter binding of compound in presence of ERα-pp. LC/MS spectra of tethering screen results for chemical moiety 2 conjugated to 14-3-3σ (C42) apo (46%) or ERα-pp bound (59%); protein-disulfide conjugate mass: 26781 Da. LC/MS dose response curves showing percentage of protein conjugate formation for titrations of disulfides to 14-3-3σ (C42) apo and bound to ERα-pp, were measured. Dose-response curve demonstrates tighter binding of compound in presence of ERα-pp, were measured. Dose-response curve of Fragment 1, which induces ERα binding, were measured (J. Am. Chem. Soc. 2019, 141, 8, 3524-3531, FIG. 2a-2b, supporting information). Small molecules stabilize 14-3-3σ/Erα-pp binding. Schematic of experimental design and Plot of anisotropy (mean+SD) for 14-3-3σ (C42) titrations to fluorescein-Erα-pp and saturating (100 μM) Frag001, Frag002, FC-A (natural product fusicoccane-A), or DMSO control, showed a 40-fold increase of the 14-3-3σ (C42)/Erα-pp binding affinity in the presence of Frag002 (FIG. 6). Schematic of experimental design and Plot of anisotropy (mean+SD) for titrations of Frag001, Frag002, FC-A (natural product fusicoccane-A), or DMSO control to fluorescein-Erα-pp and 1 μM 14-3-3σ (C42) (J. Am. Chem. Soc. 2019, 141, 8, 3524-3531, FIG. 3a-3b). Dose-response curves were obtained by MS by analyzing % tethering for titrations of Fragment 2 to 14-3-3σ apo (− peptide;) or bound to different interaction partner-derived peptide motifs; ERα-pp ( ), TASK3-pp ( ), ExoS ( ) or TAZ-pp ( ), starting from 1 mM (J. Am. Chem. Soc. 2019, 141, 8, 3524-3531, FIG. 4b). LC/MS screening data of hits for 14-3-3σ (C42)+/−ERα-pp was collected. The deconvoluted LC/MS spectra of disulfide fragments bound to 14-3-3σ (C42) alone or in complex with ERα-pp were determined with 14-3-3σ (C42) expected mass: 26509 Da, βME capped mass: 26585 Da, protein-disulfide conjugate mass: between 26752-26919 Da. Fragments bound preferentially in the presence of ERα-pp: 959996 (1), 916971, and 917137 were termed ‘cooperative’. Fragments that bound similarly in the presence and absence of ERα-pp: 917884 (2), 917105 (3), 917929 (4), 917599 (5), 917805, and 957838 were termed ‘neutral’. Compounds with bold numbers are described in the main text (J. Am. Chem. Soc. 2019, 141, 8, 3524-3531, supporting information). Fragment 6 displays cooperativity with ERα-pp when bound to 14-3-3σ(C42), but not (C45). Fragment-protein conjugate formation (% Tethered) to 14-3-3σ(C42) or 14-3-3σ(C45) versus concentration of 6, both in presence or absence of ERα-pp was tested. Fluorescence anisotropy (r) of fluorescein-ERα-pp versus concentration of 6 or FC-A binding to 14-3-3σ(C42) or 14-3-3σ(C45) was tested. The EC50 values for 6 bound to 14-3-3σ(C42)/ERα-pp or 14-3-3σ(C45)/ERα-pp were determined to be 340 nM and 687 nM, respectively, though the maximum anisotropy was much lower for 14-3-3σ(C45) (J. Am. Chem. Soc. 2019, 141, 8, 3524-3531, supporting information). Binding of non-covalent derivative of disulfide hit fragment to 14-3-3σ and ERα-pp were tested with samples labeled ‘protein/peptide’ containing 10 μM 14-3-3σ, 15 μM ERα-pp, 200 μM ligand. Fragment binding is confirmed in a T1p experiment for relaxation observed after 10 ms and 200 ms: the self-relaxation of 11% in the absence of protein-peptide is increased to 42% in the presence of protein-peptide, due to binding to 14-3-3/ERα-pp. Binding is also seen in waterLOGSY spectra by the change in signal sign recorded in presence and absence of protein-peptide complex. The spectrum shows the waterLOGSY spectrum of 14-3-3/ERα-pp alone (J. Am. Chem. Soc. 2019, 141, 8, 3524-3531, supporting information). Binding affinity determined from fluorescence anisotropy (r) titration curves for 14-3-3σ(C42) binding to various fluorescein-labeled peptides derived from partner proteins ERα (ERα-pp 0.3 μM EC₅₀), TASK3 (TASK3-pp 0.2 μM EC₅₀), ExoS 11.1 μM EC₅₀), and TAZ (TAZ-pp 2.2 μM EC₅₀). EC₅₀ values were obtained from nonlinear fitting of the data (J. Am. Chem. Soc. 2019, 141, 8, 3524-3531, supporting information).

### Example 2: Experimental Details and Compound Characterization

Protein expression and purification. The 14-3-3 cr isoform with a truncated C-terminus after T231 (AC; to enhance crystallization) and an N-terminal His6-tag was expressed in NiCo21 (DE3) competent E. coli (New England biolabs Inc) from a pPROEX HTb expression vector. Site-directed mutagenesis to obtain double mutants C38N/N42C and C38N/S45C was performed using the QuickChange Lightening site-directed mutagenesis kit (Agilent Technologies) following manufacturer's instructions. C38N was selected since asparagine is the most prevalent amino acid at that position across the 14-3-3 family. Constructs were confirmed by DNA sequencing. After transformation following manufacturer's instructions, single colonies were picked to inoculate 30 mL pre-cultures (LB), which were added to 1.5 L 2XYT medium after overnight growth at 37° C., 250 rpm. Expression was induced upon reaching OD₆₀₀ 0.5-0.6 by adding 400 μM IPTG. After overnight expression at 18° C., 140 rpm, cells were harvested by centrifugation at 8000 rpm and resuspended in lysis buffer (50 mM Tris, pH 8.0, 300 mM NaCl, 10 mM imidazole, 5 mM MgCl₂, 1 mM PMSF, 250 μM TCEP). The His6-tagged proteins were first purified by Ni-affinity chromatography (HisTrap HP column, GE) (Elution buffer 50 mM Tris, pH 8.0, 300 mM NaCl, 250 mM imidazole, 250 1 . . . LM TCEP), followed by His-tag cleavage by TEV protease during dialysis (25 mM HEPES pH 7.5, 200 mM NaCl, 5% glycerol, 10 mM MgCl2, 250 1 . . . LM TCEP) overnight at 4° C. The flow-through of a second HisTrap column was subjected to final purification step by size-exclusion chromatography (Superdex75, GE) (SEC buffer 25 mM HEPES pH 7.5, 100 mM NaCl, 10 mM MgCl2, 250 1 . . . LM TCEP). The protein was concentrated to ˜60 mg/mL, analyzed for purity by SDS-PAGE and Q-Tof LC/MS and aliquots flash-frozen for storage at ˜80° C.

Peptide sequences. Peptides for disulfide trapping were purchased from Elim Biopharmaceuticals, Inc. (Hayward, Calif.) Sequences were as follows: Ac-KYYITGEAEGFPA{pT}V-COOH (ERα-pp) (SEQ ID NO:3); Ac-RRK{pS}V-COOH (TASK3-pp) (SEQ ID NO:4); Ac-RSH{pS}SPASLQLGT-CONH₂ (TAZ-pp) (SEQ ID NO:5); Ac-SGHGQGLLDALDLAS-CONH₂ (ExoS) (SEQ ID NO:6). ERα-pp for X-ray crystallography and fluorescein-labeled peptides were ordered from GenScript Biotech Corp. Sequences were: Ac- or 5-FAM-AEGFPA{pT}V-COOH (8mer ERα-pp) (SEQ ID NO:7) and 5-FAM-labeled sequences as above for TASK3-pp, TAZ-pp and ExoS (i.e., 5-FAM labeled SEQ ID NO:4, 5-FAM labeled SEQ ID NO:5, and 5-FAM labeled SEQ ID NO:6).

Disulfide Tethering screening and data processing. The primary screening was performed by incubating the target with individual compounds in a 384-well plate format. A custom library of 1600 disulfide-containing fragments of the UCSF Small Molecule Discovery Center (SMDC), synthesized as previously reported, was available as 50 mM stock solutions in DMSO. (496, 497) For screening, 14-3-3σ wild-type and Cys-mutants were diluted to 100 nM in buffer (10 mM Tris, 100 μM betamercaptoethanol (βME), pH 8.0) and plated in 384-well plates (15 μL/well). 30 nL of each fragment was pinned from the library master plates into the protein samples using a Biomek FX (Beckman) to give a final concentration of 100 μM. The duplicate experiments additionally contained 200 nM ERα-pp. The reaction mixtures were incubated for 3 hours at RT before being subjected to LC/MS (I-class Acquity UPLC/Xevo G2-XS Quadrupole Time of Flight mass spectrometer, Waters). Data collection and automated processing followed a custom workflow, as previously described. (492) All compounds described in the text were from the same lot as the original screening material.

Dose-Response LC/MS experiments. Disulfide tethering dose-response analysis used the same procedures as primary screening, with the exception that the βME concentration was 1 mM, and compounds were titrated from 5-50 mM in 2-fold serial dilutions in DMSO, then 400 nL of the compound was transferred to 10 μL protein solution for final concentrations 0.1-2000 μM and 4% DMSO. For the dose-response of 2 in the presence of TAZ-pp (150 μM), a 5 minute chromatography step was employed to separate the hydrophobic peptide from the 14-3-3 before ionization.

Fluorescence Anisotropy. Fluorescein-labeled peptides, 14-3-3 protein, FC-A (10 mM stock solution in DMSO) and disulfide fragments (50 mM stock solutions in DMSO) were diluted in buffer (10 mM HEPES pH 7.5, 150 mM NaCl, 0.1% TWEEN-20, 1 mg/mL Bovine Serum Albumine (BSA; Sigma Aldrich)). Final DMSO concentration in the assay was always 1%. Dilution series of 14-3-3 protein or fragments were made in black, round-bottom 384-microwell plates (Corning) in a final sample volume of 10 μL in triplicates. Fluorescence anisotropy measurements were performed directly and after overnight incubation at room-temperature, using a Tecan Infinite F500 plate reader (filter set λₑₓ: 485±20 nm, λₑₘ: 535±25 nm). Data reported are at endpoint. EC₅₀ values were obtained from fitting the data with a four-parameter logistic model (4PL) in GraphPad Prism 6.

X-Ray Crystallography. 14-3-3 protein (470 μM; 12.5 mg/mL) was mixed with ERα-pp (1:2 molar stochiometry; 940 μM) and incubated in buffer (20 mM HEPES pH 7.4, 2 mM MgCl₂, 2 mM βME overnight at 4° C. before setting up for sitting drop crystallization in MRC crystallization plates (Swissci) with a custom crystallization liquor-grid (0.095 M HEPES (pH 7.1, 7.3, 7.5, 7.7), 0.19 M CaCl₂, 5% glycerol, 24-29% PEG 400). Crystals grew at 4° C. within 4 days. Soaking of crystals was performed by mixing 0.4 μL disulfide fragments from 50 mM stock solutions in DMSO with 2 mM βME in 3.6 μL mother liquor, and adding this to crystal-containing drops. Soaked crystals were fished after overnight incubation and flash-frozen in liquid nitrogen.

### Example 3: Additional Binding Site Compound Screening

Small-molecule modulation of protein-protein interactions (PPIs) is one of the most promising strategies for drug discovery and a very active field in chemical biology. Especially the field of targeted PPI inhibition has matured into a successful area (498), whereas the opposite strategy of PPI stabilization has been largely a domain of serendipity and retrospective elucidation of modes-of-action (499,500). However, as the examples of the immunomodulatory drug (IMiD) Lenalidomide (Revlimid®) and the immunosuppressant Rapamycin (Rapamune®) show, the approach of PPI stabilization can be tremendously successful. In contrast to inhibitors of both PPIs and more traditional drug targets, how to design and optimize such PPI stabilizers in a systematic approach is not well established yet. One successful technology for a ‘bottom-up’ strategy for drug candidate identification is fragment-based drug discovery (FBDD). (501) Here, very small, (100-250 Da), low-affinity (mM range) protein binders are identified by various biophysical methods (NMR, X-Ray, DSF, FP, SPR) and subsequently be optimized towards higher potency. (501) A variation of this approach is ‘tethering’⁵, where a native or engineered cysteine is used as covalent anchor for disulfide-containing fragments to ‘trap’ their weak binding for identification by mass spectrometry. We have recently shown how the introduction of cysteines at the interface of the adapter protein 14-3-3 and a peptide derived from the nuclear receptor ERα can be used to identify the first 14-3-3/ERα PPI-stabilizing fragments. (502)

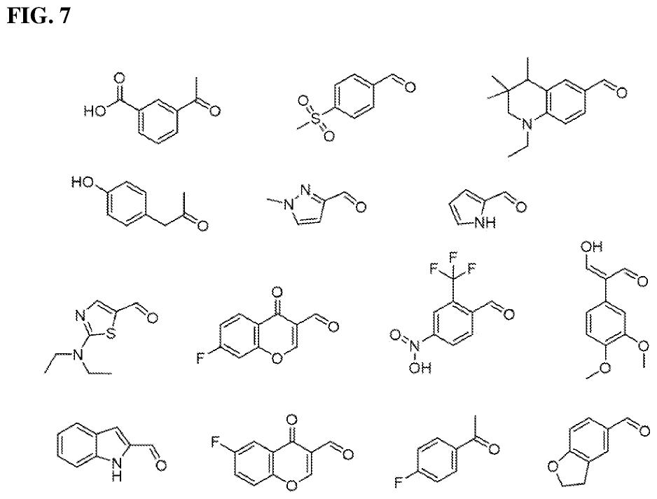

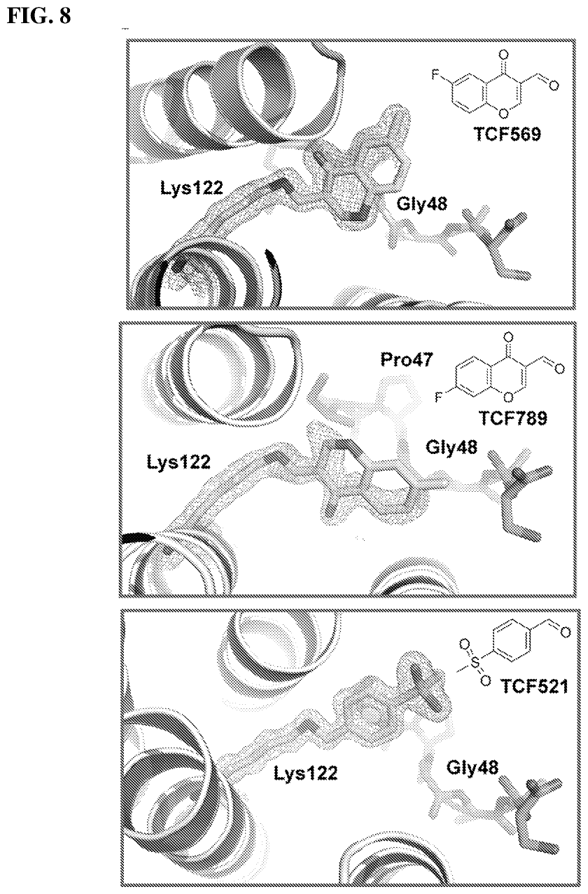

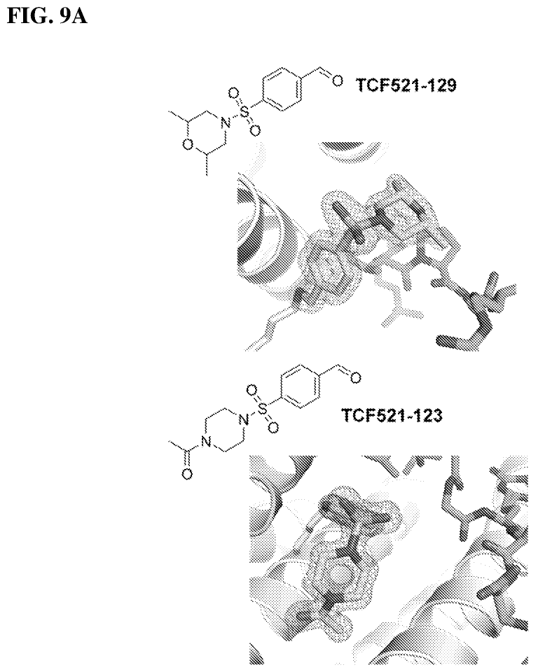

Due to the fact that in most of the cases there is no native cysteine near the prospected target site, the covalent mode of binding in tethering is only used to trap the low-affinity fragment. In order to bind to the surface of the wild-type protein, the covalent anchor needs to be removed in subsequent optimization steps. Since this is also the case with our previous example of fragments covalently bound to the 14-3-3/ER, we asked the question if it was possible to target a non-cysteine residue in an interface of 14-3-3 with a partner protein peptide. Our recently solved structure of 14-3-3 in complex with the interaction motif from the p65 subunit of NFκB offers such an opportunity as it displays an accessible lysine residue (Lys122) that locates closely to Ile46 and Pro47 of the NFκB peptide. In addition, we have shown that DP005—a semisynthetic derivative of Fusicoccin A—stabilizes this interaction and binds to the interface of 14-3-3 and the peptide. In order to evaluate the possibility of covalently targeting Lys122 in the 14-3-3/NFκBp65 complex, we assembled a small library of aldehyde-bearing fragments (FIG. 7) and soaked these individually into crystals of the binary 14-3-3 complex. Additional density observed after X-ray diffraction and data analysis identified binding of three fragments to Lys122 (FIG. 8). Fragments TCF569 and TCF789 showed only partial coverage by electron density indicating a relative low occupancy (FIG. 8). In addition, these molecules can have a tendency of ring rearrangements and display properties of PAINS. In contrast, TCF521, a 4-mesylbenzaldhyde, is completely covered by the density map allowing the unambiguous positioning of the molecule (FIG. 8). Binding of TCF521 to the complex is most probably more advantageous due to a better fit of the single benzyl ring arranging in a hydrophobic contact with the side chain of Ile46 compared to the more bulky and rigid double-ring systems displayed by TCF569 and TCF789. For this reason and the overall good chemical tractability, we decided to continue with TCF521 as the scaffold to design a small library of extended fragments.

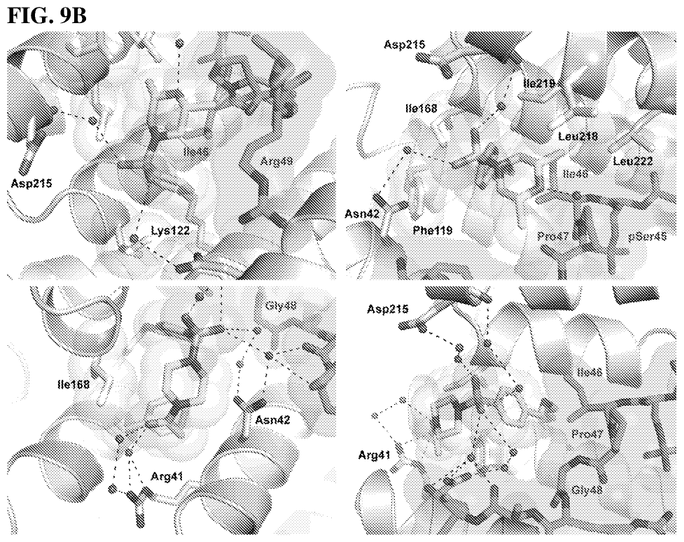

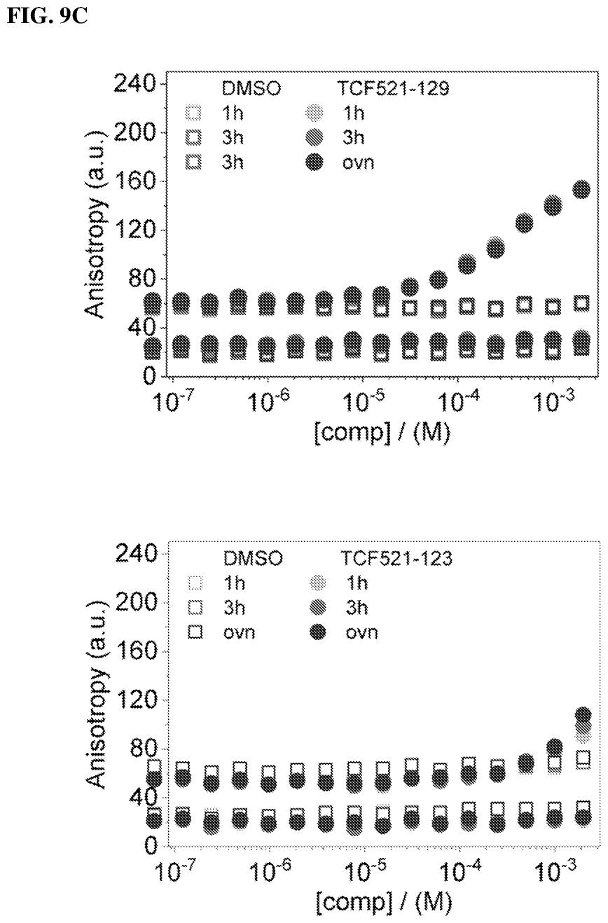

As the primary assays to test for binding and activity we used X-ray protein crystallography (crystal soaking) and a fluorescence polarization assay (FP) employing a fluorophore-labelled (FITC) version of the NFκBp65 peptide. After some rounds of analogues testing, we were able to identify the molecules' electrochemical properties that are enabling the lysine attach to the carbonyl feature of the fragment. One interesting observation was that while many extended fragments showed a clear and well-defined binding in the x-ray structure, predominantly those that showed an enhanced contact surface with the peptide showed stabilization activity in the functional FP assay. TCF521-129 shows a clear electron density (FIG. 9A) that unambiguously point the sulfonamide extensions into the direction of the NFκB peptide. This reflects a principal feature of orthosteric PPI stabilization which is based on the direct, simultaneous physical interaction of the stabilizer with both protein partners.

In addition to the hydrophobic interaction of the primary phenyl ring of TCF521-129 with the side chain of Ile46, the 2,6-dimethylmorpholine substituent sits on top of this side chain and additionally contacts with one of its methyl groups Pro47 and Gly48. The opposite methyl is engaging a hydrophobic patch in the ‘roof’ of the 14-3-3 groove comprised of Leu218, Ile219, and Leu22 (FIG. 9B, upper row). The sulfonamide oxygens of TCF521-129 each establish a water-mediated contact with 14-3-3, one to the side chain of Asn42, the other to the main-chain oxygen of Asp215 (FIG. 9B, upper row). The interaction of the sulfonamide with 14-3-3 is slightly different between TCF521-129 and TCF521-123 fragments, which can be explained by the deviation of the position of this group when bound to 14-3-3. Both fragments establish a polar opposite methyl is engaging a hydrophobic patch in the ‘roof’ of the 14-3-3 groove comprised of Leu218, Ile219, and Leu22 (FIG. 9B, upper row). The sulfonamide oxygens of TCF521-129 each establish a water-mediated contact with 14-3-3, one to the side chain of Asn42, the other to the main-chain oxygen of Asp215 (FIG. 9B, upper row). The interaction of the sulfonamide with 14-3-3 is slightly different between TCF521-129 and TCF521-123 fragments, which can be explained by the deviation of the position of this group when bound to 14-3-3. Both fragments establish a polar contact with the side chain of Asn42. In the case of TCF521-129 the contact to Asn42 is mediated by a water as is the interaction with the main-chain carbonyl oxygen of Asp215 (FIG. 9B, upper row). Finally, the sulfonamide oxygens in TCF521-123 are engaged via a more complex water network with both Asn42 and Asp214 of 14-3-3 as well as main-chain oxygens of Arg50 and Ser51 of the p65 peptide (FIG. 9B, upper row). The potential PPI stabilizing activity of both fragments were tested in a fluorescence polarization assay measuring the binding of a fluorescently-labelled (FITC) NFκBp65 peptide to 14-3-3 in the presence of an increasing concentration of the fragment. TCF521-129 stabilizes the interaction of the peptide with 14-3-3 with an EC₅₀ of 370 μM, whereas TCF521-123 display only a very weak at the highest concentrations tested (FIG. 9C).

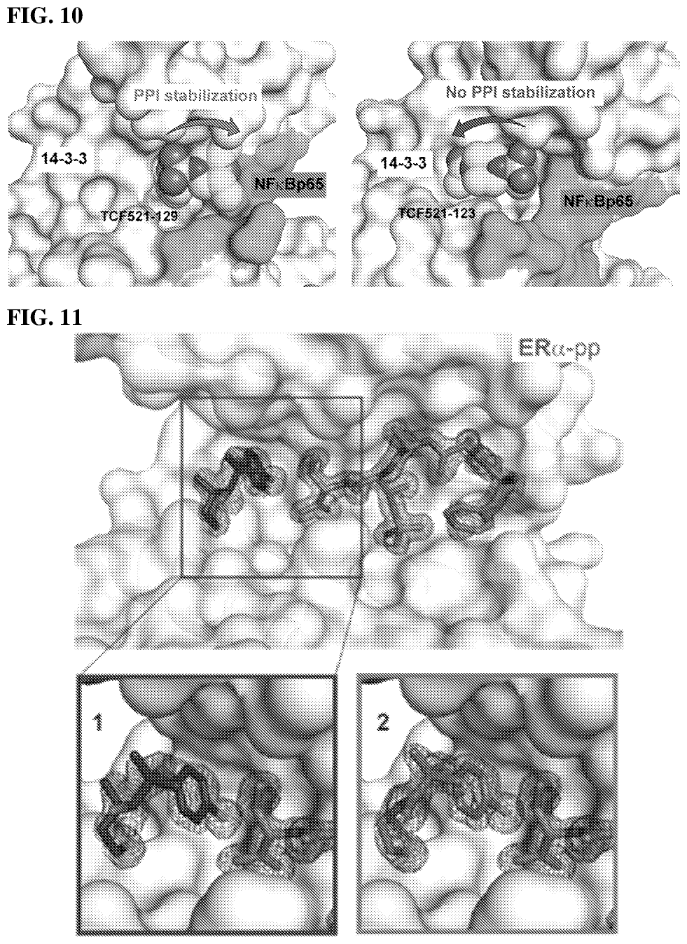

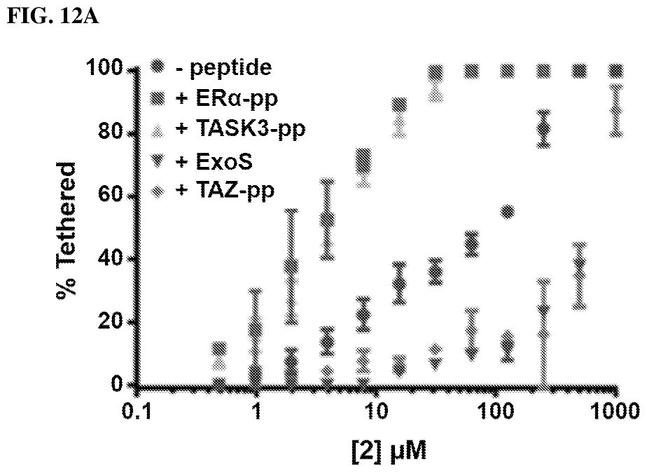

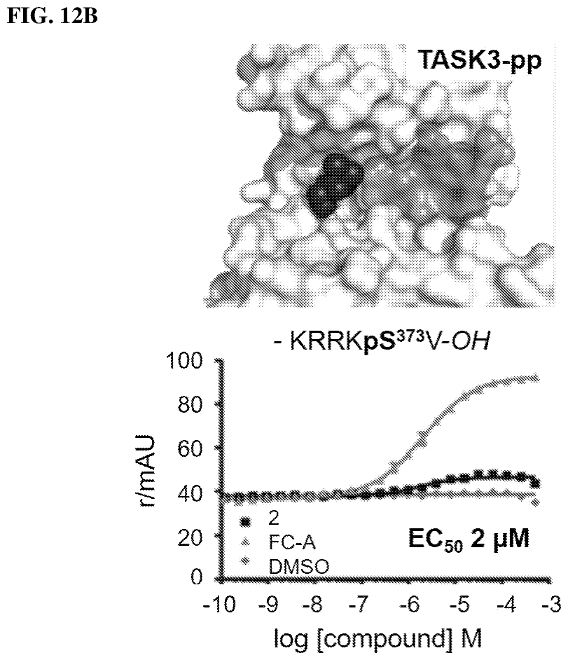

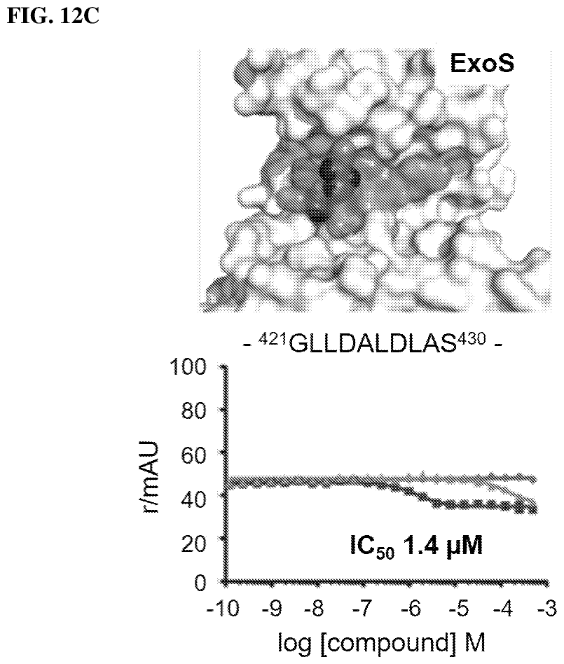

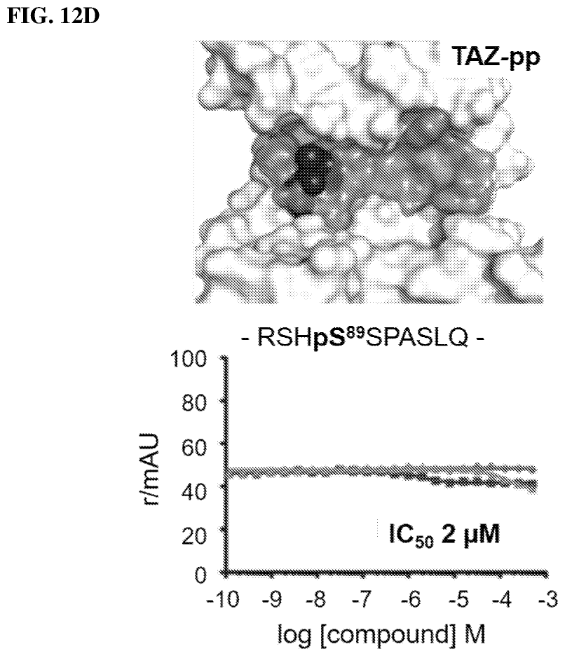

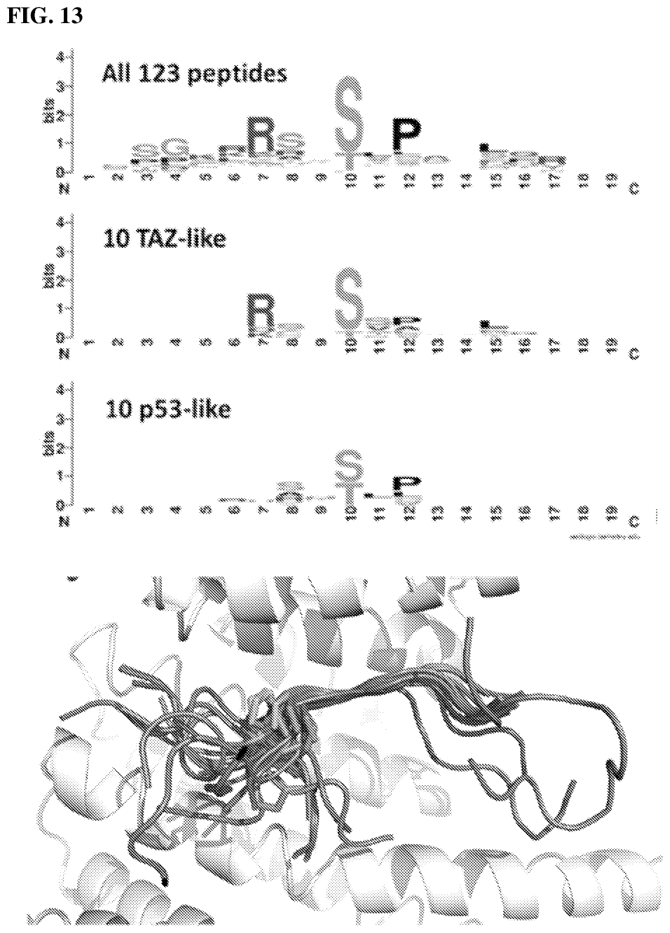

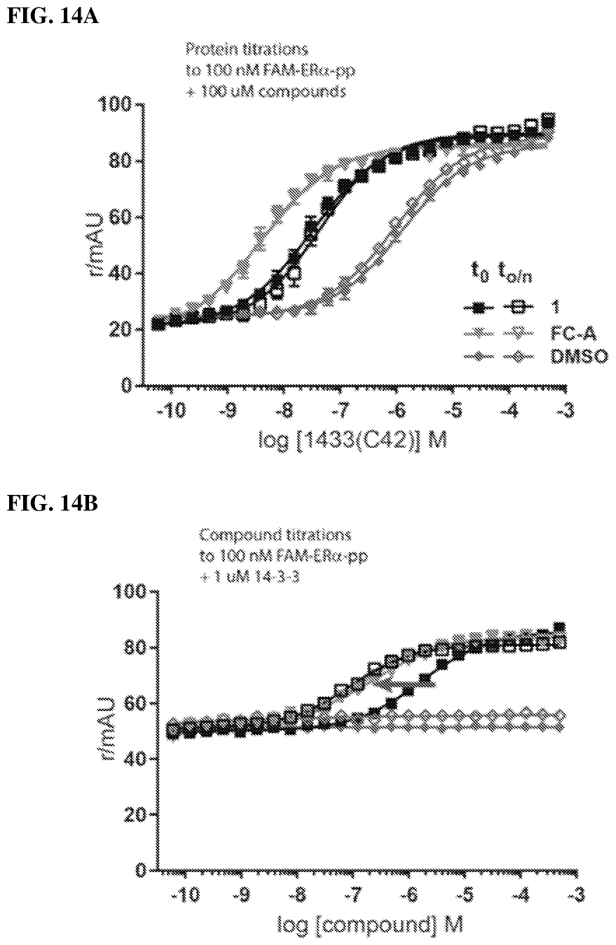

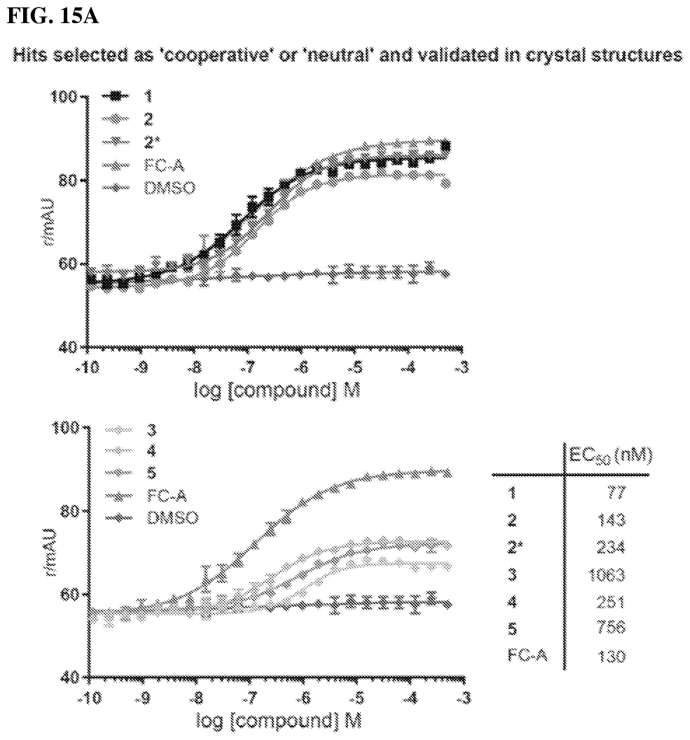

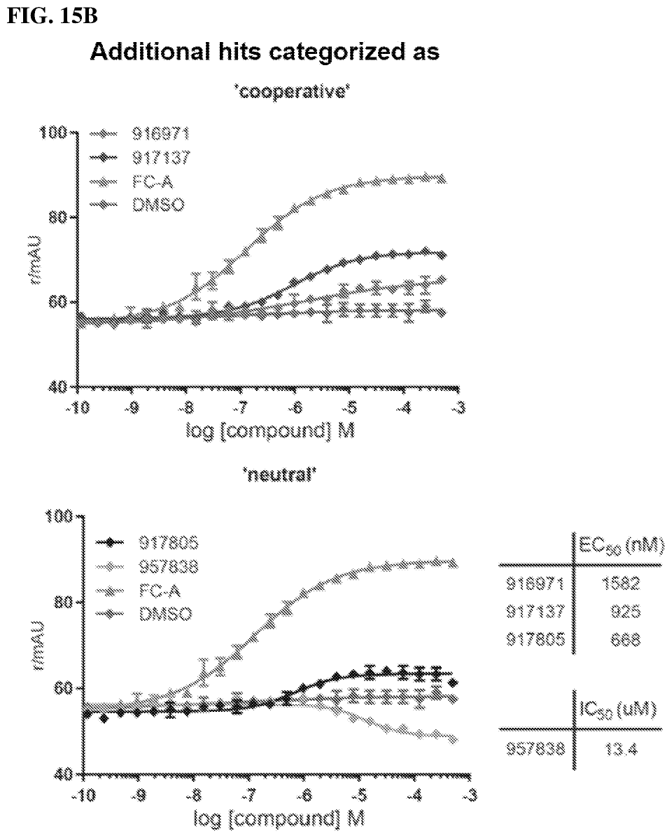

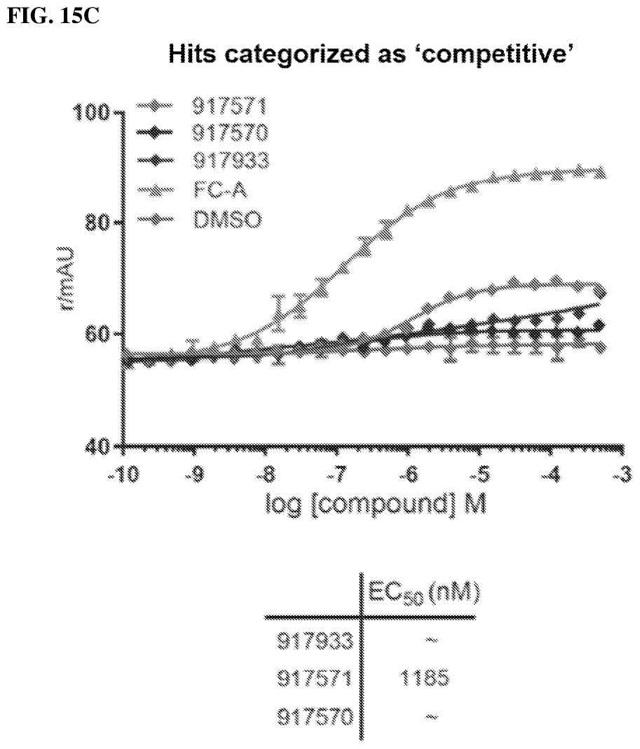

The main difference in binding to the interface of the p65 peptide and 14-3-3 between the PPI-stabilizing and non-stabilizing fragments is the way the substituent of the sulfonamide arranges either towards the peptide (TCF521-129) or to the opposite direction (TCF521-123), enhancing the interaction with 14-3-3 (FIG. 10). For the overall goal of stabilizing the interaction of the two partner proteins and increase the cooperativity of the extended fragments, the priority at this stage should be to increase the contacts with the peptide rather than with 14-3-3.

Ultimately, the potency of a PPI stabilizer will depend on the distribution between the binding energy to the two proteins. From theoretical considerations this should be in the ideal case shared equally. However, in the case of 14-3-3 PPIs two considerations speak in favor of aiming for extended contacts with the peptide. i) 14-3-3 proteins are structurally highly conserved among all seven human isoforms and interact with several hundred protein partners⁷. This means that the structural diversity of the interface to which an orthosteric stabilizier binds and which is the basis for specificity of the compound, is contributed largely by the target protein. ii) The 14-3-3 binding motifs of the partner proteins are localized exclusively in disordered regions of the target protein that only undergo a disorder-to-order transition when these peptide stretches bind to 14-3-3. This results in very low binding affinities of the PPI stabilizer to the target protein when it is not complexed with 14-3-3. One of the main advantages of orthosteric PPI stabilizers is the fact that their affinity to the individual partner proteins is low and increases by several orders of magnitude when they bind to the target complex (505,506). The combination with the aforementioned disordered nature of the 14-3-3 recognition sequences combines thus two favourable features of 14-3-3 PPIs as small molecule targets which should enable the development of specific and potent synthetic molecules for this protein class.

### Example 4: Additional Compound Characterization

X-Ray Crystallography Data Collection and Refinement

Diffraction data were collected on in-house X-ray diffraction system (Equipped with Rigaku MicroMax-003 sealed tube X-ray source and Rigaku Dectris PILATUS3 R 200K detector), at the Deutsche Electronen Synchrotron (DESY, PETRA-III beamline) or Swiss Light Source (PXII beamline). Initial processing of all datasets was done using Pipedream from GlobalPhasing. (507) First, Autoproc (508) ran XDS (509) for data indexing and integration, and AIMLESS (510,511) for scaling. Then Phaser (512) was used for limited molecular replacement using PDB ID 4JC3 as template. Finally, Buster (513) was used for structure refinement. Upon completion of the pipedream run, presence of soaked ligands was verified by visual inspection of the Fo-Fc and 2Fo-Fc electron density maps in Coot. (514) If electron density corresponding to the soaked ligand was present, its structure and restrains were generated using eLBOW (515) before final model building and refinement was done using phenix.refine (516,517) and Coot. See Table 2 for data collection and refinement statistics. The structures were submitted to the PDB with IDs 6HHP, 6HMT, 6HKF, 6HKB, 2HN2 and 6HMU.

Synthetic Procedure

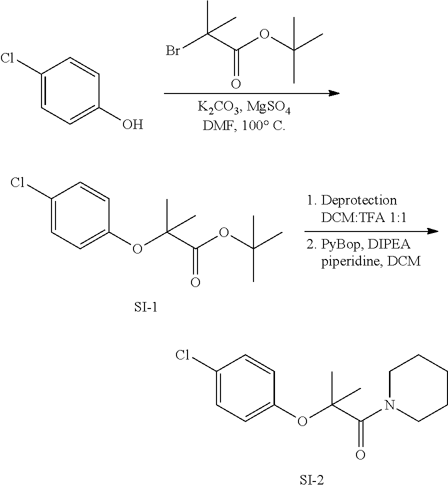 [SMILES: Oc1ccc(Cl)cc1 ; CC(C)(C)OC(=O)C(C)(C)Br ; CC(C)(C)OC(=O)C(C)(C)Oc1ccc(Cl)cc1 ; "SI-1" ; CC(C)(Oc1ccc(Cl)cc1)C(=O)N1CCCCC1 ; "SI-2"]

Tert-butyl 2-(4-chlorophenoxy)-2-methylpropanoate (SI-1) To a solution of 4-chlorophenol (375 mg, 2.9 mmol) in DMF (10 mL) was added tert-butyl 2-bromo-2-methylpropanoate (1952 mg, 8.8 mmol), K₂CO₃ (1612 mg, 11.7 mmol) and MgSO₄ (351 mg, 2.9 mmol). The reaction mixture was heated to 100° C. and stirred overnight under nitrogen. After, the mixture was cooled and diluted with H₂O. The aqueous solution was extracted three times with EtOAc. The combined organic layers were dried on MgSO₄. After removal of the solvent in vacuo, the crude product was purified by flash chromatography (0-20% EtOAc/Hexane, 25 CV). ¹H NMR (400 MHz, DMSO-d6) δ 7.32 (d, J=8.9 Hz, 1H), 6.81 (d, J=9.0 Hz, 1H), 1.49 (s, 6H), 1.39 (s, 9H).

2-(4-chlorophenoxy)-2-methyl-1-(piperidin-1-yl)propan-1-one (SI-2) SI-1 was dissolved in 1:1 DCM/TFA (3 mL) and stirred at RT for 5 h. The solvent was removed in vacuo. To the product (0.4 mmol) re-dissolved in DCM (2 mL) was added PyBop (1.25 eq.), and after shaking for 10 min, DIPEA (40.6 ul) and piperidine (3 eq.). The reaction mixture was stirred overnight. The crude was purified on prep-TLC using 60/40 EtOAc/Heptane. Removal of solvent in vacuo resulted in the final product. ¹H NMR (400 MHz, DMSO-d6) δ 7.35-7.30 (m, 2H), 6.84-6.77 (m, 2H), 3.67 (s, 2H), 3.46 (s, 2H), 1.53 (s, 6H), 1.46 (s, 2H), 1.37 (s, 2H), 1.15 (s, 2H). MS (ESI) calc. for C₁₅H₂₀ClNO₂ [M] 281.78; observed [M]⁺ 282.

NMR Spectroscopy

In one dimensional ligand-observed experiments, waterLOGSY (518) and T1p′³ (10/200 ms) spectra were recorded to obtain binding information for the fragment SI-2 to the 14-3-3cT/ERα-pp complex. Protein concentration 10 μM, ERα-pp concentration 15 μM, fragment concentration 200 μM. Experiments were performed at 296 K on a 600 MHz Bruker AVANCE III spectrometer, equipped with a triple-resonance cryogenic probe head. All samples were diluted in buffer (25 mM d-Tris, 100 mM NaCl, 1 mM TCEP, pH 7.4).

TABLE 2
XRD statistics

| 14-3-3σ Δc/ERα-pp | C42-959996 | | C42-917884 | | C42-917599 | |
|---|---|---|---|---|---|---|
| PDB ID | 6HHP | | 6HMT | | 6HKB | |
| Data collection | | | | | | |
| Wavelength (Å) | 1.54 | | 1.033 | | 1.54 | |
| Resolution (Å) | 45.39-1.80 | (1.84-1.80) | 66.22-1.10 | (1.12-1.10) | 66.37-1.70 | (1.73-1.70) |
| Space group | C2221 | | C2221 | | C2221 | |
| Unit cell | 81.87 | | 81.93 | | 82.23 | |
| | 112.21 | | 112.43 | | 112.39 | |
| | 62.41 | | 62.39 | | 62.44 | |
| Total reflections^{a} | 166665 | (6652) | 1359280 | (27522) | 151020 | (7696) |
| Unique reflections^{a} | 26771 | (1423) | 114386 | (4256) | 32176 | (1681) |
| Redundancy^{a} | 6.2 | (4.7) | 11.9 | (6.5) | 4.7 | (4.6) |
| Completeness (%)^{a} | 99.3 | (89.4) | 98.1 | (74.5) | 99.9 | (100.0) |
| Average^{I/σ}_{(I)}^{a} | 30.8 | (7.6) | 22.3 | (1.9) | 15.4 | (7.5) |
| Wilson B-factor | 6.1 | | 9.3 | | 6.3 | |
| CC_{1/2}^{a, b} | 0.999 | (0.972) | 1.000 | (0.657) | 0.995 | (0.967) |
| R_{sym}^{a, c} | 0.048 | (0.191) | 0.052 | (0.910) | 0.075 | (0.188) |
| Rₘₑₐₛ^{a, d} | 0.052 | (0.214) | 0.054 | (0.991) | 0.084 | (0.212) |
| Refinement | | | | | | |
| Reflections (refinement) | 26753 | | 114341 | | 32128 | |
| Reflections (R-free) | 1302 | | 2898 | | 1674 | |
| Non-hydrogen atoms | 2234/321 | | 2334/312 | | 2256/339 | |
| (overall/solvent) | | | | | | |
| R_{work} (%) | 17.1 | | 18.1 | | 17.7 | |
| R_{free} (%) | 21.5 | | 18.4 | | 20.0 | |
| RMS (bonds)/(angles) | 0.006/0.827 | | 0.004/0.792 | | 0.006/0.883 | |
| Average protein B-factor | 10.94 | | 10.41 | | 10.79 | |
| Ramachandran: favored/ | 98.3/0.0 | | 98.3/0.0 | | 98.3/0.0 | |
| outliers (%) | | | | | | |
| Clashscore | 2.12 | | 2.01 | | 1.32 | |
| 14-3-3σ Δc/ERα-pp | C42-917929 | | C42-917105 | | C45-957782 | |
| PDB ID | 6HKF | | 6HN2 | | 6HMU | |
| Data collection | | | | | | |
| Wavelength (Å) | 1.54 | | 1.54 | | 1.033 | |
| Resolution (Å) | 45.46-1.80 | (1.84-1.80) | 66.27-1.70 | (1.73-1.70) | 66.18-1.20 | (1.22-1.20) |
| Space group | C2221 | | C2221 | | C2221 | |
| Unit cell | 82.02 | | 82.06 | | 81.85 | |
| | 112.53 | | 112.36 | | 112.40 | |
| | 62.47 | | 62.44 | | 62.58 | |
| Total reflections^{a} | 167745 | (6546) | 148994 | (7386) | 1133888 | (39164) |
| Unique reflections^{a} | 26955 | (1428) | 30678 | (1530) | 90076 | (4343) |
| Redundancy^{a} | 6.2 | (4.6) | 4.9 | (4.8) | 12.6 | (9.0) |
| Completeness (%)^{a} | 99.3 | (89.4) | 95.8 | (92.1) | 99.9 | (98.5) |
| Average^{I/σ}_{(I)}^{a} | 33.6 | (8.3) | 17.3 | (7.6) | 28.2 | (2.9) |
| Wilson B-factor | 7.2 | | 5.9 | | 10.1 | |
| CC_{1/2}^{a, b} | 0.999 | (0.973) | 0.997 | (0.973) | 1.000 | (0.838) |
| R_{sym}^{a, c} | 0.043 | (0.176) | 0.066 | (0.197) | 0.049 | (0.757) |
| Rₘₑₐₛ^{a, d} | 0.047 | (0.198) | 0.074 | (0.221) | 0.051 | (0.802) |

| | | | |
|---|---|---|---|
| Refinement | | | |
| Reflections (refinement) | 26935 | 30631 | 90056 |
| Reflections (R-free) | 1292 | 1604 | 4535 |
| Non-hydrogen atoms | 2141/226 | 2212/297 | 2320/365 |
| (overall/solvent) | | | |
| R_{work} (%) | 18.1 | 17.8 | 18.7 |
| R_{free} (%) | 21.1 | 20.8 | 20.6 |
| RMS (bonds)/(angles) | 0.003/0.59 | 0.006/0.793 | 0.005/0.844 |
| Average protein B-factor | 11.18 | 10.49 | 10.58 |
| Ramachandran: favored/ | 98.3/0.0 | 98.3/0.0 | 98.3/0.0 |
| outliers (%) | | | |
| Clashscore | 1.59 | 1.59 | 3.88 |

^{a}Number in parentheses is for the highest resolution shell used in the refinement
^{b}CC½ = Pearson's intra-dataset correlation coefficient, as described by Karplus and Diederichs.(520)
^{c}Rsym = ΣₕΣₗ | Iₕₗ − <Iₕ> |/ΣₕΣₗ<Iₕ>, ^{where} Iₕₗ is the intensity of the l^{th} observation of reflection h and <Ih> is the average intensity of reflection h
^{d}Rmeas = Σₕ | √(nₕ/nₕ − 1))Σₗ | Iₕₗ − <Iₕ> | |/ΣₕΣₗ<Iₕ>, where nh is the number of observations of reflection h
^{e}Correlation of experimental intensities with intensities calculated from refinded model, as described by Karplus and Diederichs. (520)

### Example 5: Fragment-Based Protein-Protein Interaction Stabilizers Via Imine-Based Tethering

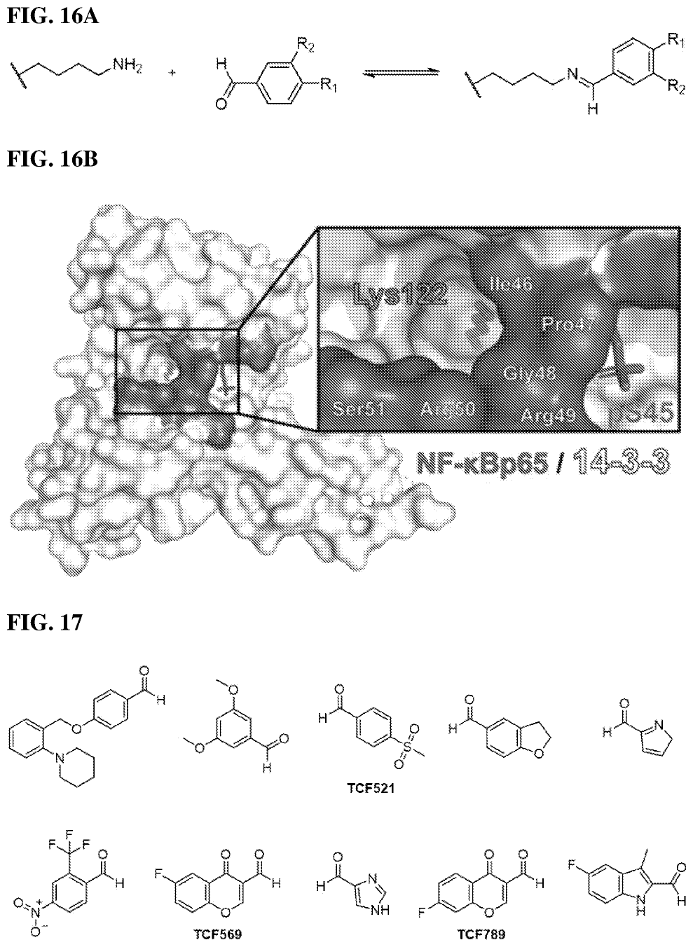

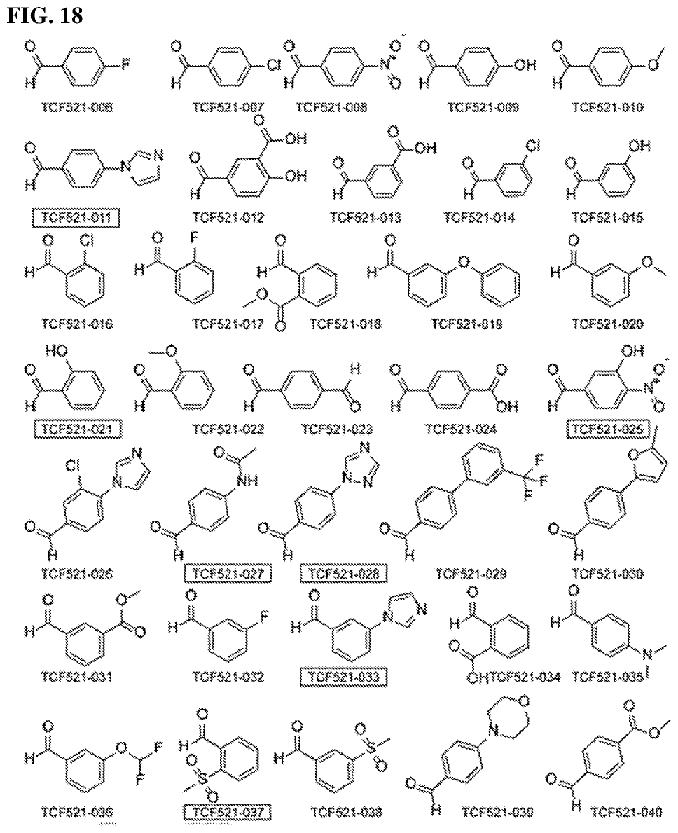

Lysines constitute a large percentage of the proteinogenic amino acids, with concomitant covalent and dynamic covalent drug targeting approaches developed for this amino acid. Aldehydes forming aldimine bonds provide attractive entries for targeting lysine sidechains, but have typically only been successful when the imine bond was intrinsically stabilized by flanking chemical functionalities which trap the imine bond via an intramolecular hydrogen bond. Nevertheless, we reasoned the reversible nature of imine bonds to be of high potential for tethered FBDD of PPI complexes. The formation of non-trapped aldimines would potentially aid in identifying fragments with beneficial contacts to the target pocket, as templating effects would facilitate aldimine bond formation. Here we show the use of dynamic covalent fragments which stabilize a protein complex, using imine chemistry as covalent anchor. Illustrated using the 14-3-3/NF-κB interaction, a high value drug target, we reveal how a composite PPI binding pocket featuring the hydrophobically buried Lys122 provides entry to selective PPI stabilizers (FIGS. 16A-16B). Notably, hit compounds are specific Lys122 binders, affording exquisite control over localization. Additionally, our study reveals that only those fragments that feature enhanced contacts with the NF-κB element, rather than with 14-3-3 alone, provide the best starting points as molecular glues.

14-3-3σ, exemplary as one of the seven 14-3-3 isoforms, features 18 mostly solvent exposed lysine residues. In silico analysis of the local pKa values (Table 3) with the Rosetta webtool showed that Lys122 and Lys159 feature the lowest predicted pKa's both around 10, suggesting these two residues to be most amenable to imine bond formation. Lys122 is of particular interest, as the amino acid is located within a predominately hydrophobic region of the 14-3-3 phosphopeptide binding groove (FIG. 16B). Lys122 is part of the so-called ‘Fusicocin binding pocket’—a preferred drug targeting pocket for 14-3-3 PPI stabilization - and thus ideally positioned to explore for fragment-based PPI stabilization via imine-based tethering. The crystal structure of 14-3-3 in complex with the p65 subunit of NF-κB offers an excellent opportunity for fragment crystal soaking, also because the hydrophobic microenvironment around Lys122 is further extended by three hydrophobic residues of p65, Ile46, Pro47 and Gly48 (FIG. 16B).

TABLE 3
Analysis of pKa values of lysine residues based
on the p65_45^{R}/14-3-3σΔC crystal structure

| # Fields: | p65/14-3-3σΔC | |
|---|---|---|
| Residue N Chain | IpKa | pKa |
| LYS 9 A | 10.4 | 10.2 |
| LYS 11 A | 10.4 | 11.3 |
| LYS 27 A | 10.4 | 11.2 |
| LYS 32 A | 10.4 | 10.6 |
| LYS 49 A | 10.4 | 10.6 |
| LYS 68 A | 10.4 | 11.2 |
| LYS 75 A | 10.4 | 10.5 |
| LYS 77 A | 10.4 | 10.2 |
| LYS 87 A | 10.4 | 10.3 |
| LYS 109 A | 10.4 | 10.5 |
| LYS 122 A | 10.4 | 10 |
| LYS 124 A | 10.4 | 10.4 |
| LYS 140 A | 10.4 | 10.5 |
| LYS 141 A | 10.4 | 11.2 |
| LYS 159 A | 10.4 | 10 |
| LYS 160 A | 10.4 | 11 |
| LYS 195 A | 10.4 | 10.8 |
| LYS 214 A | 10.4 | 10.7 |

Initially, we assembled a small collection of 10 aldehyde-bearing fragments (FIG. 17) and soaked these fragments individually into crystals of the binary p65/14-3-3 complex. Following X-ray diffraction data analysis, additional density was observed for three fragments binding to Lys122: 1 (TCF521), 2 (TCF569) and 3 (TCF789). 2 and 3 showed partial electron density coverage and at least three other lysine residues elicited extra electron density, indicating non-specific reactivity of these compounds.

In contrast, 1 was completely covered by the electron density map allowing the unambiguous elucidation of the molecular orientation. No secondary binding site was detected for 1, testifying to its potential for selective targeting of the Lys122. The balanced reactivity and specificity of 1 for Lys122 likely relates to the aldehyde moiety being activated by the electron-withdrawing sulfonyl moiety in combination with templating effects based on hydrophobic contacts of the benzyl ring with the side chain of Ile46. Replacement of the aldehyde moiety with related functional groups like acid, alcohol, amine, ketone and methyl impeded binding in the crystal structure, highlighting the essential contribution of the imine bond formation. An extended aldehyde fragment library with various ring substitutions on the benzaldehyde core was subsequently tested in the crystal screening setup. Interestingly, only those fragments featuring an electron withdrawing group, activating the aldehyde for imine formation, showed electron density in the crystal structures. Importantly though, all compounds again specifically bound to Lys122. These results further support the importance of a balanced activation of the aldehyde for effective but specific imine formation with Lys122. We also soaked the ortho-hydroxy variant of 1, featuring a hydrogen bond donor group typically used for imine bond trapping. However, of all investigated aldehydes this was the only one inducing crystal cracking potentially caused by pan-labeling of the majority of the lysine residues.

Given the well-defined binding mechanism of 1 and its chemical tractability, we sought to grow this compound into a stabilizer of the p65/14-3-3 interaction. To this end, we designed a focused library of extended fragments, of which derivatives 4 (TCF521-123) and 5 (TCF521-129), showed highly interesting binding characteristics. Briefly, 4 and 5 were accessed via a sulfonyl amide coupling of 4-formylbenzenesulfonyl chloride with 4-acetylpiperazin-1-yl (4) or 2,6-dimethyl-morpholine (5), respectively (Scheme 1).

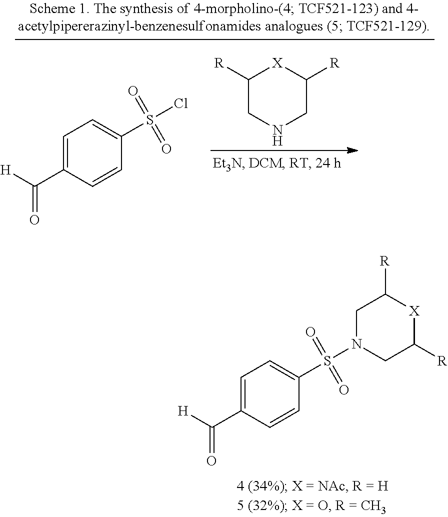

Structural data on the binding of both compounds was acquired using crystal soaking experiments. The additional electron density was again specifically confined to Lys122 and both compounds were completely covered by the electron density map. The aldehyde functionalities of 4 and 5 account for a continuous electron density with the Lys122 side chain, verifying the covalent imide bond formation. The coupling of a single compound to 14-3-3 was also verified with mass spectroscopy after reductive amination of the imine bond. The aromatic element of the benzaldehyde ring of both compounds engages in hydrophobic contacts with Ile46 of p65. Both sulfonamide groups make additional water mediated contacts with 14-3-3 via Asn42 and the backbone of Asp215. These interactions are analogous to those found for the starting fragment 1 and clarify the basal binding affinity of these fragments to the 14-3-3 scaffold.

A significant difference between both fragments was found regarding the orientation of their sulfonyl amide head groups. These newly inserted functionalities, as compared to 1, adopt opposite conformations within the PPI interface. The substituted morpholino ring system of 5 actively engages with elements of the p65 peptide, while the piperazine functionality of 4 adopts an opposite orientation and points away from the p65 element. The sulfonamide oxygens in 4 are engaged in a complex water network with additional water-mediated contacts to Arg41 of 14-3-3 and the Arg50 and Ser51 main-chain carbonyls of the p65 peptide. 5 is engaged in a less pronounced water network and its morpholino group bends off to p65. In addition, one of the methyl groups of 5 makes additionally contacts with Pro47 and Gly48 of p65. The other methyl group is engaging in hydrophobic contacts with the ‘roof’ of the 14-3-3 groove comprised of residues Leu218, Ile219, and Leu222. As ultimate proof of the potential stabilizing capacity of these Lys122-specific, imine forming compounds, biochemical PPI stabilization studies were performed. Compound titrations of 4 and 5 with a fluorescently-labeled monovalent p65 peptide and 14-3-3 protein induced a concentration dependent increase in fluorescence anisotropy (FA), indicative for compound driven complex stabilization. Whereas both compounds induce an increase in anisotropy, 5 is active at lower concentrations and shows a stronger increase in anisotropy. The stabilizing effect of the compounds was quantified by titrating 14-3-3 to a bivalent p65 peptide and multiple constant concentrations of compound. Decreasing apparent dissociation constants (K_{D}) due to increasing compound concentrations imply complex stabilization. Comparing the K_{D} of the DMSO control and of the highest compound concentration reveals a stabilization factor (SF) of SF=3 for 4 and SF=8 for 5.

The observed stabilizing effect of 4 is probably solely caused by the hydrophobic contact between the benzaldehyde ring and Ile46 of p65. The tilted conformation of 4 has the overall effect of an increased distance between the benzaldehyde ring and Ile46 of the peptide, potentially weakening this hydrophobic contact. The orientation of 5 overlays more precisely with that of the initial hit 1, reaching the full potential of this hydrophobic contact.

The combined structural and biochemical data reveal that the additional contacts made by the morpholino ring of 5 with both the 14-3-3 protein and the p65 peptide are beneficial for the ternary small molecule-stabilized complex. In contrast, the additional contacts of 4 by virtue of its piperazine functionality and the extensive contacts with the water network are exclusively engaged with 14-3-3. While such observations are highly valuable towards affinity optimization and selectivity considerations, here specifically these do not contribute to p65/14-3-3 stabilization. As an adapter protein, 14-3-3 binds to multiple other interaction partners. Importantly, 4 and 5 are not able to stabilize the TAZ/14-3-3, ERα/14-3-3, nor the p53/14-3-3 interaction. These are three representative 14-3-3 client proteins covering a typical interaction partner binding in an elongated manner in the binding groove (TAZ), one with a phosphorylated C-terminus (ERα) and a partner with a bent conformation alike the one p65 but with a bulky and charged amino acid in +1 position of the phosphorylation site (p53). The transient nature of the imine bond prevents binding competition of compound and TAZ peptide binding, while both ERα and p53 engage Lys122 in polar bonds, hence prevent imine formation. The specific molecular nature of p65, making a sharp turn out of the 14-3-3 binding pocket, governed by Ile46, Pro47, and Gly48 of p65, provides access to the Lys122 in a uniquely generated composite hydrophobic pocket. This reflects a principal feature of orthosteric PPI stabilization which is based on the direct, simultaneous physical interaction of the stabilizer with both protein partners.

We have developed compounds that stabilize the 14-3-3/p65 complex, using a site-directed fragment screening approach. This screening approach is unique to other covalent aldehyde chemical probes, which are reliant on trapping moieties. The lack of trapping moieties enables us to exploit templating effects caused by the binding of the p65 subunit. The unique pKa profile of Lys122, in combination with templating effects of the partner peptide facilitates the specific aldimine bond formation with Lys122. Further, we demonstrate how initial fragments can be rapidly developed into extended stabilizing fragments which elicit promising activity. Further we show that the unique interface of 14-3-3/p65 enables the development of selective fragments. This concept provides valuable starting points for further PPI drug development.

### Example 6: Additional Compound Characterization

Protein Expression and Purification

14-3-3 proteins were expressed in BL21(DE3) cells with pPROEX HTb vectors encoding for the indicated isoforms. Cells were grown to an OD₆₀₀=0.8-1 in TB media and expression was induced with 0.4 mM IPTG overnight at 18° C. After harvesting the cells by centrifugation (10.000×g, 15 min), they were resuspended in lysis buffer (50 mM Tris/HCl pH8, 300 mM NaCl, 12.5 mM imidazole, 2 mM β-mercaptoethanol). A homogenizer was used for cell lysis, the lysate was cleared via centrifugation (40.000×g, 30 min). The cleared lysate was applied to Ni-NTA-columns and eluted with 250 mM imidazole (50 mM Tris/HCl pH8, 300 mM NaCl, 250 mM imidazole, 2 mM β-mercaptoethanol). For the full length 14-3-3γ, the imidazole was removed via dialysis, the protein rebuffered (25 mM HEPES pH7.5, 100 mM NaCl, 10 mM MgCl₂, 0.5 mM Tris(2-carboxyethyl)phosphine) and stored at −80° C. For the 14-3-3σΔC (last 17 amino acids of flexible C-terminus were removed) for crystallography, the His6-tag was removed following standard procedures of TEV cleavage; the TEV was removed with Ni-NTA-columns. For highest purity necessary for crystallography, the protein was additionally applied to size exclusion chromatography (20 mM HEPES pH7.5, 150 mM NaCl, 2 mM β-mercaptoethanol) and stored at −80° C.

Note to the use of 14-3-3 isoforms: 14-3-3σΔC was only used for crystallography because of its enhanced potential to grow high resolution crystals. For all other techniques the 14-3-3γ isoform was used due to its beneficial binding to the p65 epitope. All residues of the p65/14-3-3 interface are conserved throughout all human 14-3-3 isoforms, so that the observed contacts in the crystal structures are translatable to all isoforms.

X-Ray Crystallography

Binary crystals with NF-κBp65 peptide (Sequence: EGRSAG pS45 IPGRRS, C-terminus: amidation; N-terminus: acetylation (SEQ ID NO:9)) and 14-3-3σΔC were grown as follows: 14-3-3σΔC at a concentration of 12 mg/ml was mixed in a 1:2 ratio with the acetylated NF-κBp65 peptide in 20 mM HEPES pH7.5, 2 mM MgCl2, 2 mM 3-mercaptoethanol and incubated at 4° C. overnight. Then it was mixed in 1:2 ratio with precipitation buffer (95 mM HEPES pH7.5, 27-28% PEG400, 190 mM CaCl₂, 5% glycerol) in the wells of a hanging drop crystallography plate. The reservoir was filled with 500 μL precipitation buffer. Crystals grew within two weeks and were directly flash frozen in liquid nitrogen for data acquisition.

For soaking experiments compounds in DMSO stock solutions were directly added to fully grown crystals to a final compound concentration of 10 mM (≤1% DMSO) in the crystal solution. After seven days the crystals were harvested and measured either on a home source, P11 beamline of PetraIII (DESY campus, Hamburg, Germany) or i-03/i-24 beamline of the diamond light source (Oxford, UK). For data integration the xia2/DIALS pipeline was utilized followed by molecular replacement with MolRep using the NF-κBp65/14-3-3 binary structure as search model (PDB ID: 6QHL). Model building was done in iterative cycles with Coot, Refmac5 and phenix.refine. For ligand preparation, the fragment SMILES were transformed to 3D models using elbow of the phenix suite. Figures were generated with PyMOL© (V2.0.6, Schrodinger LLC). The crystal structures were uploaded to the PDB server with the following PDB IDs: 6YOW, 6YP2, 6YOY, 6YOX, 6YP3, 6YP8, 6YPL, 6YPY, 6YQ2.

Mass Spectrometry

Reductive amination of 4 (TCF521-123) and 5 (TCF521-129) and following LC/MS analysis of the complex was performed as follows: 50 μM of 14-3-3γ and/or 1 mM of monovalent p65 peptide and 500 μM of compound were incubated in 10 mM HEPES pH 7.4, 150 mM NaCl for 1 h at RT, then an 1000×excess of NaBH₃CN was added (fresh stock solution with 6 mg/ml). The mixture was incubated for 1 h at RT before the reaction was stopped with 0.1% formic acid, diluting the mixture by 1:100.

For all the samples were applied to a hybrid quadrupole time-of-flight (QTOF) LC/MS system, with 1 μL injection volume. The chromatogram was measured on an Agilent Polaris C18-A 100×2.00 mm column over 8 min with a water/acetonitrile (+0.1% formic acid) gradient of 15-60% acetonitrile/0.1% formic acid followed by 2 min washing with 15% acetonitrile acetonitrile/0.1% formic acid (flowrate 0.3 ml/min, column temperature 60° C.). For MS data acquisition a full scan with 150-2000 m/z was performed and data analysis was performed with the MassLynx software. For deconvolution of the mass/z spectra the MaxEnt1 function of the MassLynx software was applied to the 4 most abundant peaks of the mass distributions. The output mass range was set to 30-40 kDa with a resolution of 0.1 Da/channel. As damage model the “simulated isotope pattern” was applied, whereby the blur width was determined by measuring the peak width of the most abundant peak at half of its height (typically 0.3 Da). All graphs were prepared with OriginLab 2019.

Fluorescence Anisotropy Assays

Fluorescence Anisotropy was measured in Corning 384 well plates (black, round bottom, low binding) with the Tecan Infinite 500 plate reader (FITC dye: excitation 485 nm, emission 535 nm; TAMRA dye: excitation 535 nm, emission 590 nm) in FA buffer (10 mM HEPES pH 7.4, 150 mM NaCl, 0.1% Tween20). The plates were measured after 3 h incubation at RT. The concentration of the peptide coupled to the fluorescent tracer was kept constant as follows: FITC-βAla-p65 c=100 nM, TAMRA-Ahx-p53 peptide c=10 nM, FITC-βAla-TAZ c=10 nM, FITC-O1Pen-ERα c=10 nM. Peptide sequences are listed in Table 4.

TABLE 4
Overview of peptide sequences. Shown are
the names of the peptides used, the N-terminal
fluorescent dyes and the corresponding linker,
and the sequence.

| Peptide | N-terminal | |
|---|---|---|
| name | modification | Sequence |
| p65 | FITC-βAla | EGRSAG pS45 IPGRRS |
| monovalent | | (SEQ ID NO: 9) |
| p65 | FITC-βAla | EGRSAG pS45 IPGRRS |
| bivalent | | GSGGGSGPSDREL pS |
| | | EPMEFQ (SEQ ID NO: 10) |
| TAZ | FITC-βAla | RSH pS89 SPASLQ |
| | | (SEQ ID NO: 11) |
| P53 | TAMRA-Ahx | SRAHSSHLKSKKGQS |
| | | TSRHKKLMFK pT387 |
| | | EGPDSD-COOH |
| | | (SEQ ID NO: 12) |
| ERa | FITC-OlPen | AEGFPA pT594 V-COOH |
| | | (SEQ ID NO: 13) |

For compound titrations, the 14-3-3γ concentrations was constant at concentration of 1/3 of the K_{D} of the binary complex (assay concentration for p65: 50 μM, TAZ: 0.1 μM, Era: 0.1 μM, p53: 0.3 μM) and the compound was titrated in a 1:1 dilution series with a highest concentration of 2 mM.

For protein titrations, the compound and peptide concentration were constant as indicated; the protein was titrated in a 1:1 dilution series starting from 400 μM. For 2D titrations the compound was diluted in a 1:1 dilution series in DMSO prior to the protein titrations to keep the DMSO concentration constant throughout the assay.

General information. All commercial chemicals were used as received. Reagents were used without further purification unless otherwise noted.

TLC analysis was performed on TLC aluminum sheets, silica gel layer, ALUGRAM SIL G UV254, 20×20 cm by MACHEREY-NAGEL. TLC plates were analysed by UV fluorescence (254 nm).

UHPLC-MS analysis was performed using UPLC Agilent Technologies 1290 Infinity coupled with Agilent Technologies 6120. Quadrupole LC/MS DAD detector. Column: ACQUITY UHPLC BEH C18 (1.7 μm) 2.1 mm×50 mm. Temperature: 40° C. Detection: DAD+MS/6120 Quadrupole. Injected volume: 1 μL. Flow: 1.2 mL/min. Solvent A: Water+0.1% Formic Acid. Solvent B: Acetonitrile+0.1% Formic Acid. Gradient: 0 min 2% B; 0.2 min 2% B; 2.0 min 98% B; 2.2 min 98% B; 2.21 min 2% B; 2.5 min 2% B.

Preparative HPLC was performed using UPLC Agilent Technologies 1260 Infinity coupled with Agilent Technologies 6120 Quadrupole

LC/MS. Column: Waters XBridge Prep C18 5 μm OBD 19×150 mm. Detection: DAD+MS/6120 Quadrupole. Flow: 32 mL/min. Solvent A: Water+0.1% Formic Acid. Solvent B: Acetonitrile+0.1% Formic Acid. Gradient: 0 min 77% A/23% B; 1 min 77% A/23% B; 9 min 16% A/84% B; 9.01 min 2% A/98% B; 11 min 2% A/98% B.

¹H NMR and ¹³C NMR spectra were recorded on a Bruker 300 MHz spectrometer at ambient temperature. The chemical shifts are listed in ppm on the 5=scale and coupling constants were recorded in Hertz (Hz). Chemical shifts are calibrated relative to the signals corresponding of the non-deuterated solvent (CHCl₃: 5=7.26 ppm for 1H and 77.16 for 13C). Abbreviations are used in the description of NMR data as follows; chemical shift (5=ppm), multiplicity (s=singlet, d=doublet, t=triplet, m=multiplet, bs=broad singlet), coupling constant (J=Hz).

Synthesis of Selected Products.

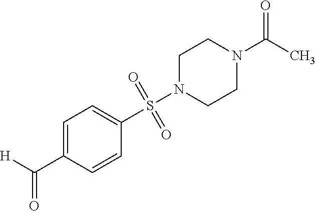 [SMILES: CC(=O)N1CCN(S(=O)(=O)c2ccc(C=O)cc2)CC1]

### 4-[(4-acetylpiperazin-1-yl)sulfonyl]benzaldehyde (4, TCF521-123)

To a solution of 0.29 mmol (1 Eq.) 1-Acetylpiperazine in 1 mL of DCM were added 0.88 mmol (3 Eq.) of Triethylamine. After stirring at room temperature for 10 min., a solution of 0.29 mmol (1 Eq.) 4-formylbenzene-1-sulfonyl-chloride in 1 mL of DCM was added. The reaction was stirred at room temperature for 24 hours. After complete consumption of the starting materials—monitored by TLC (DCM/MeOH 9:1) and UHPLC-MS—the mixture was added of 1 mL of NaHCO₃ saturated solution. After separation, the organic layer was dried and concentrated under pressure. The compound was purified by preparative HPLC. Obtained 30 mg (34% yield) of 4 (TCF521-123) with purity 99% by UHPLC-MS as a white solid. UHPLC-MS (ESI+APCI) m/z calcd. for C₁₃H₁₆N₂O₄S [M+H]⁺=297. Found: 297. Retention time: 1.01 min. ¹H NMR (300 MHz, CDCl₃) δ=10.11 (s, 1H), 8.05 (d, J=8.44 Hz, 2H), 7.90 (d, J=8.30 Hz, 2H), 3.70 (t, 2H), 3.55 (t, 2H), 3.04 (m, 4H), 2.02 (s, 3H) ppm. ¹³C NMR (75 MHz, CDCl₃) δ=190.39, 166.63, 140.45, 139.00, 130.06, 128.11, 45.85, 45.57, 45.46, 40.48, 20.98 ppm.

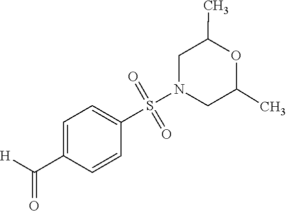 [SMILES: CC1CN(S(=O)(=O)c2ccc(C=O)cc2)CC(C)O1]

### 4-[(2,6-dimethylmorpholin-4-yl)sulfonyl]benzaldehyde (5, TCF521-129)

To a solution of 0.29 mmol (1 Eq.) 2.6-Dimethylmorpholine in 1 mL of DCM were added 0.88 mmol (3 Eq.) of Triethylamine. After stirring at room temperature for 10 min., a solution of 0.29 mmol (1 Eq.) 4-formylbenzene-1-sulfonyl-chloride in 1 mL of DCM was added. The reaction was stirred at room temperature for 24 hours. After complete consumption of the starting materials—monitored by TLC (DCM/MeOH 9:1) and UHPLC-MS—the mixture was added of 1 mL of NaHCO₃ saturated solution. After separation, the organic layer was dried and concentrated under pressure. The compound was purified by preparative HPLC. Obtained 26.6 mg (32% yield) of 5 (TCF521-129) with purity 99% by UHPLC-MS as a white solid. UHPLC-MS (ESI+APCI) m/z calcd. for C₁₃H₁₇NO₄S [M+H]+=284. Found: 284. Retention time: 1.29 min. ¹H NMR (300 MHz, CDCl₃) δ=10.12 (s, 1H), 8.06 (d, J=8.47 Hz, 2H), 7.91 (d, J=8.26 Hz, 2H), 3.70 (m, 2H), 3.60 (d, J=10.14 Hz, 2H), 1.97 (m, 2H), 1.13 (d, J=6.27 Hz, 6H) ppm. ¹³C NMR (75 MHz, CDCl₃) δ=190.49, 140.57, 138.81, 129.95, 128.09, 71.12, 50.49, 18.39 ppm.

### Example 7: Exploration of a 14-3-3 PPI Pocket by Covalent Fragments as Stabilizers

Here, we present the results of a study into the properties of disulfide-tethered ligands and analyze both the affinity of fragments at the 14-3-3 PPI pocket and the cooperativity observed for fragments engaging a specific subpocket. The position of the cysteine residue used for screening by disulfide trapping was found to be crucial. Comparing covalent fragments tethered to different cysteine residues along the rim of the pocket provided insight into pocket ligandability by fragments. Residue C42 was suitable for finding stabilizing fragments, whereas fragments bound more potently—but not cooperatively—to a cysteine at position 45 (C45). Both sites yielded several co-crystal structures that provided hypotheses for binding affinity and cooperativity. Initial structure-activity relationships (SAR) were explored for the main cooperative hit tethered to 14-3-3σ(C42), aiding our understanding of the rules for 14-3-3/client stabilization by covalent fragments.

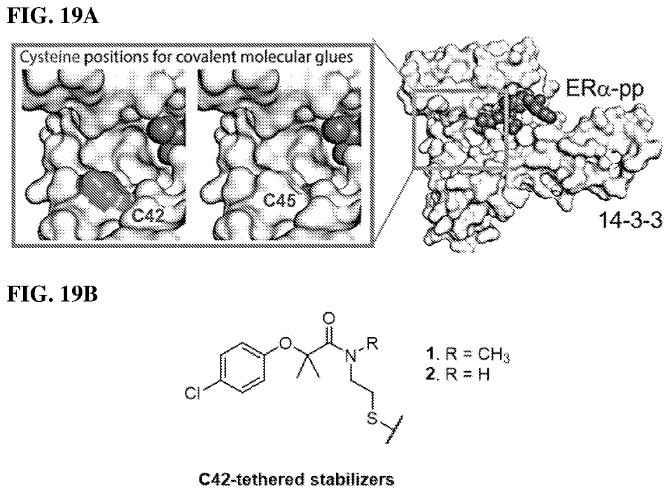

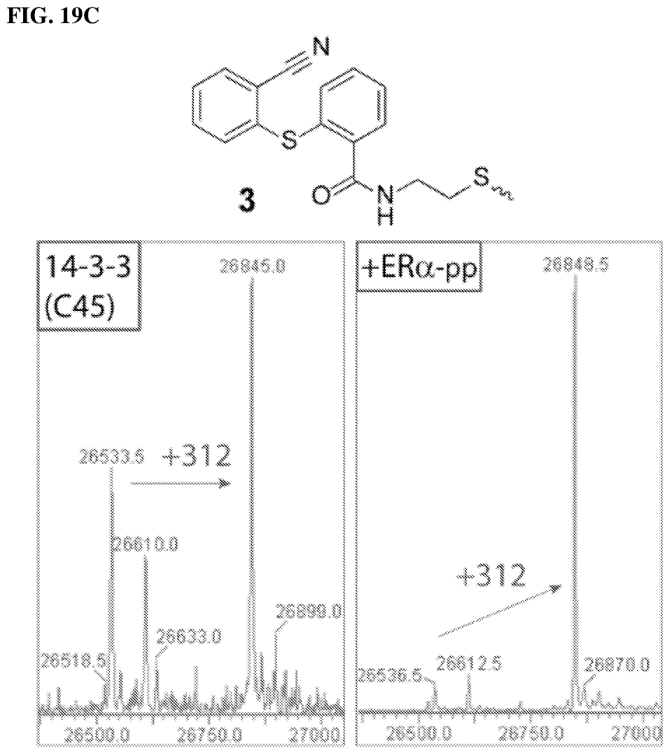

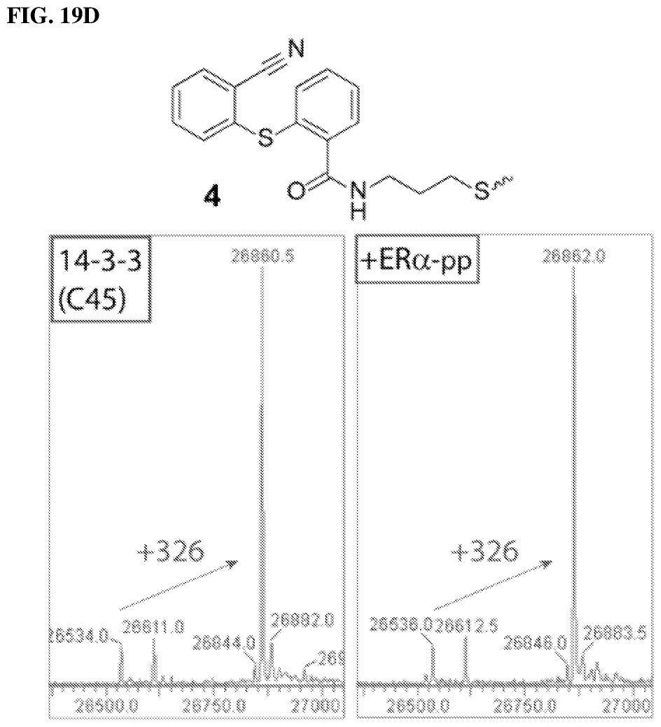

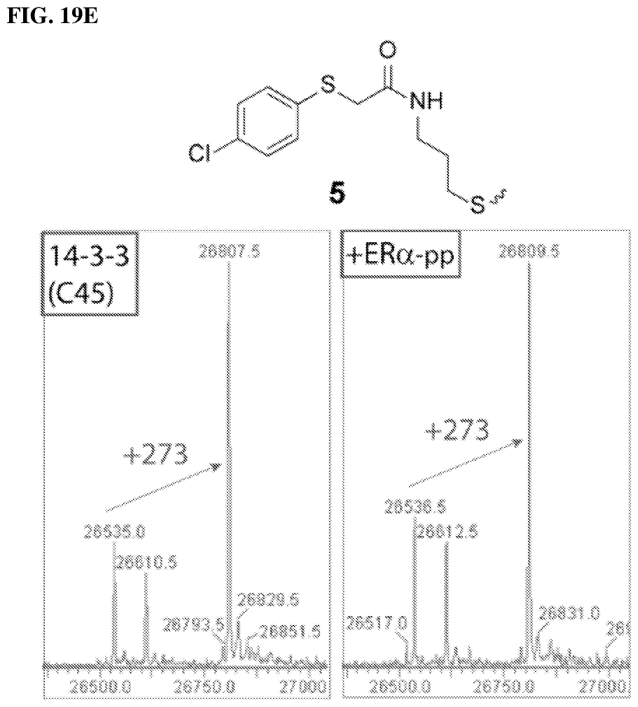

Covalent fragments tethered to 14-3-3σ C45. Covalent fragments that strongly stabilized the interaction between 14-3-3 and ERα were described previously. Briefly, in a site-directed screening approach, we varied the position of a cysteine residue serving as a reactive handle for thiol-disulfide exchange with a library of ˜1600 disulfide-containing fragments. We introduced cysteines at residues 42 and 45 on 14-3-3σ at the base of the target pocket adjacent to the C-terminus of the ERα-pp in the protein/peptide complex (FIG. 19A). Fragments 1 and 2, tethered to 14-3-3σ(C42), showed the best stabilization of the 14-3-3/ERα-pp complex (FIG. 19B); however, we also identified fragments tethered to 14-3-3σ(C45). Of these, fragment 3 was ˜50% bound to 14-3-3σ(C45) in the absence of ERα-pp and ˜90% bound to 14-3-3σ(C45) in the presence of ERα-pp, based on intact mass spectrometry (MS; FIG. 19C). Additional fragments displayed high % bound, as observed from the protein-conjugate peaks for 4 and 5 (FIGS. 19D-19E). Here, no difference was observed between 14-3-3σ(C45) apo or ERα-pp bound, indicating a strong affinity of these fragments to 14-3-3 alone and no additional influence from the ERα peptide on fragment binding. Based on these studies, there is no initial indication of PPI stabilizing or inhibiting activity.

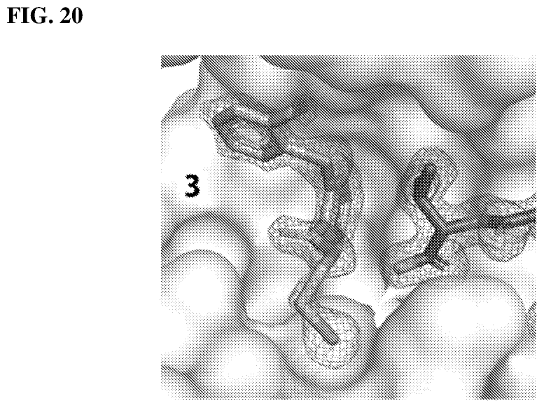

Soaking co-crystals of 14-3-3σ(C45)/ERα-pp enabled the observation of electron density for the three fragments 3-5, with the most convincing, continuous density for 3 (FIG. 20). 4 only differs from 3 by the addition of a longer alkyl chain (C3 versus C2 in 3), resulting in a less optimal binding pose in the PPI complex. Fragment 5 features a chlorophenyl moiety, as is seen in fragments 1 and 2; interestingly, while the C1 moiety is positioned identically, the phenyl ring of 5 is slightly tilted relative to 2. To determine whether fragments 3-5 stabilized the 14-3-3σ(C45)/ERα-pp complex, we measured the binding of fluorescein-labeled ERα-pp to 14-3-3σ(C45) by fluorescence anisotropy. Notably, fragments 3-5 did not induce 14-3-3σ(C45)/ERα-pp complex formation, implying no stabilization of the protein/peptide complex. This observation was particularly striking for 3, given the close proximity between the fragment and ERα-pp in the co-crystal structure (FIG. 20), and its apparent increased binding to 14-3-3σ(C45) in the presence of ERα-pp observed by mass spectrometry (FIG. 19C).

The lack of cooperativity for fragments 3-5 was confirmed in MS titration experiments, where the conjugation peak for 14-3-3σ(C45)-3 indicated >80% tethering for all concentrations of fragment 3 (100 nM-1 mM), which was not influenced by the presence of ERα-pp. Interestingly, a dose-response effect was observed for titration of 3 to 14-3-3σ(C42), where the presence of ERα-pp slightly increased %-tethering at all concentrations. This cooperative difference for 3 tethered to C45 versus C42 was further confirmed by fluorescence anisotropy, where fluorescein-labeled ERα-pp binding was enhanced upon titration of 14-3-3σ(C42) with 3 (EC₅₀ 0.9±0.11 μM). These data indicated that, in addition to the appropriate chemophore, the covalent tethering position was also important to elicit stabilization. Together, these data suggested that even though the C45-tethered fragments bound tightly to the 14-3-3 pocket, they lacked significant stabilizing activity towards the motif. Since they also did not show any inhibition towards the PPI under study, these fragments, as neutral binders, were compatible with the binary complex but did not engage the composite interface enough to drive orthosteric cooperativity. Thus, cooperativity in PPI binding is finely tuned and depends on an optimal positioning of all molecular elements.

These results illustrate that strong binding of a fragment to the PPI complex does not necessarily result in PPI stabilizing activity. Indeed, when looking in more detail at data for C42 hits, strong tethering by itself or clear density in a co-crystal structure are not per se good predictors of stabilizing activity towards 14-3-3/ERα-pp, whereas differential dose-response behavior of tethered fragments in absence or presence of ERα-pp by MS is nicely correlated with stabilization in fluorescence anisotropy. Fragment 1, for example, displays high % tethering to 14-3-3σ(C42) only in the presence of ERα-pp, which is reflected by efficient stabilization of 14-3-3/ERα-pp by 1. The co-crystal structures display similarly clear density for fragments 1 and 3, further confirming that a good binder (even to a composite pocket) is not sufficient nor necessarily predictive of PPI stabilization potential. Furthermore, the data suggest that the C45 position of 14-3-3 does not allow the fragments in our library to achieve the proper orientation to stabilize the 14-3-3σ/ERα-pp complex.

Derivatives of 14-3-3 C42-tethered stabilizers. A small library of derivatives of fragment 2 was synthesized to assess the main contributing factors to the 14-3-3/ERα-pp stabilizing activity. Co-crystal structures were obtained for eight disulfide fragments tethered to 14-3-3σ(C42) bound by ERα-pp. The most resolved electron density was observed for variants with a single para- or double meta-halogen substituent on the phenyl (6-9). The unsubstituted phenyl (10) and the p-methylphenyl (11) showed weaker electron density. A m-methoxy in addition to a p-bromo substituent resulted in well-resolved electron density for 12 whereas a combination of o-chloro and p-nitro substituents was less beneficial, resulting in electron density mainly for the phenyl group and only part of the linker for 13. Compared to the rest of the series, the phenyl ring of 13 was also rotated by 90°, directed by the o-chloro and resulting in the subsequent relocation of the linker.

Crystallographic overlays of compounds 6-12 with 2 (PDB entry 6HMT) reveals the apparent strict positioning of the halogen in the pocket, specifically when comparing single substituents on the para-position to double meta-substituents. For the double meta-substituted compounds, the molecules are reorientated so that one of the halogens (in 8 and 9) overlays with the para-chloro position of 2. The unsubstituted or p-fluorophenyl are less directing, while a p-methyl or the p-bromo/m-methoxy combination perfectly overlays with the position of 2. While all compounds occupy this same subpocket, 13 shows the most divergent binding pose.

A potential stabilization activity of 6-13 was analyzed in fluorescence anisotropy experiments by titrating 14-3-3σ(C42) and ERα-pp with the fragments. Data were collected directly after preparing the samples (t₀), and after reaching equilibrium (at endpoint, after overnight incubation, t_{o/n}). All derivatives were found to be stabilizers of the 14-3-3σ/ERα-pp complex as indicated by enhanced ERα-pp binding upon fragment titrations, displaying EC₅₀ values (173 nM-911 nM) in the same range as 2 (EC₅₀ 299±14 nM). Whereas the equilibrium was near-instantaneous for stabilization by the natural product FC-A, PPI stabilization induced by tethered fragments binding upon thiol-disulfide exchange logically displayed slower kinetics, perhaps due to the absence of βME in these experiments. The to/˜curve was shifted to the left, resulting in roughly 10-fold improved EC₅₀ values compared to to. Additionally, upper plateaus for 6, 8 and 9 reached anisotropy values similar to FC-A and 2, while for 7, 10, and 11-13 the maximum anisotropy was lower, possibly caused by a reduced stabilization of the distal region of ERα-pp. This was reflected in the crystal structures, where the sidechain of the phenylalanine at the −2 position (F591) was flexible, revealing different orientations and in two co-structures; additional density was also observed for G590.

This set of derivatives provides several valuable insights. First, it is interesting to find that all variations are tolerated and only influence stabilization activity within a 3-fold range of EC₅₀ values. Second, a halogen on the phenyl is highly beneficial for orientation into the identified subpocket, as the strongest stabilization and most resolved electron density are observed for both p-chloro (2) and p-bromo (6) and doubly substituted m,m-fluoro (8) and m,m-chloro- (9) phenyls. Finally, the constraining effect of the dimethyl moiety on the linker appears important for achieving a specific orientation.

In this work, we described covalent fragments that bound to two engineered cysteine residues near the pocket formed by the 14-3-3σ/ERα-pp complex. These fragments were identified via disulfide trapping (‘tethering’) screens that we proposed as a systematic strategy for the discovery of PPI stabilizers. Cooperative stabilization was achieved via tethering to C42, whereas tethering to C45 resulted in neutral binders based on similar chemophores. Co-crystal structures combined with biochemical binding studies revealed that tight binding alone did not necessarily guarantee effective PPI stabilization. C42 appeared to be ideally located for identifying optimal stabilizers for 14-3-3/ERα from this disulfide library. Some fragments, particularly tethered to C45, strongly bound to 14-3-3 without influencing ERα binding. Coupled with an understanding of the features that lead to PPI stabilization, these tightly bound compounds could perhaps be chemical optimized into effective stabilizers. The ability to optimize screening for local differences in target pockets is an important benefit of a reversible covalent-fragment screening strategy and further illustrates the suitability of the tethering approach to identify stabilizers for adaptive interfaces and composite PPI pockets.

### Example 8: Additional Compound Characterization

Peptide sequence. ERα peptides were purchased from GenScript Biotech Corp. The sequences, either N-terminally acetylated or FAM-labelled, was as follows:

### -AEGFPA{pT}V-COOH (SEQ ID NO:14).

Protein expression and purification. His₆-tagged 14-3-3σ proteins (full-length (FL) and ΔC) were expressed in NiCo21 (DE3) competent cells from a pPROEX HTb plasmid and purified using Ni²⁺-affinity chromatography. The ΔC variant meant for crystallization was treated with TEV protease to cleave off the His₆ tag, followed by a second Ni²⁺-affinity column and size exclusion chromatography, as described previously.²

Mass Spectrometry. Disulfide trapping dose-response titrations were performed as described in detail previously. Concentrations used: 14-3-3σ (100 nM), acetylated ERα phosphopeptide (200 nM), β-mercaptoethanol (βME, 1 mM), SMDC Monophore library (range 0.1-2000 μM in 2-fold serial dilution). Buffer: 10 mM Tris pH 8.0, sample size: 25 L, final 4% DMSO.

Fluorescence Anisotropy. Fluorescein-labeled peptides, 14-3-3 protein, FC-A (10 mM stock solution in DMSO), and disulfide fragments (50 mM stock solutions in DMSO) were diluted in buffer (10 mM HEPES pH 7.5, 150 mM NaCl, 0.1% TWEEN-20, 1 mg/mL Bovine Serum Albumine (BSA; Sigma-Aldrich)). Final 1% DMSO. Dilution series of 14-3-3 protein or fragments were made in black, round-bottom 384-microwell plates (Corning) in a final sample volume of 10 μL in triplicates. Fluorescence anisotropy measurements were performed directly and after overnight incubation at room-temperature, using a Tecan Infinite F500 plate reader (filter set λ: 485±20 nm, λₑₘ: 535±25 nm). EC₅₀ values were obtained from fitting the data with a four-parameter logistic model (4PL) in GraphPad Prism 7.

Crystallography. The 14-3-3σ protein was C-terminally truncated (ΔC) after T231 to enhance crystallization. The 14-3-3 protein and ERα phosphopeptide were dissolved in complexation buffer (25 mM HEPES pH 7.5, 2 mM MgCl₂, 2 mM beta-mercaptoethanol (βME)) and mixed in a 1:2 molar stoichiometry (protein:peptide) at a final protein concentration of 12.5 mg/mL (470 μM). The complex was set up for hanging-drop crystallization after 30 min incubation at 4° C., in a custom crystallization liquor (0.095 M HEPES (pH7.1, 7.3, 7.5, 7.7), 0.19 M CaCl₂, 24-29% (v/v) PEG 400 and 5% (v/v) glycerol). Crystals grew to a sufficient size in 7 days at 4° C.

Crystal soaks were performed by mixing 0.4 μL of 50 mM stock solutions in dimethyl sulfoxide (DMSO) with 2 mM βME in 3.6 μL mother liquor, which was then added to drops containing multiple crystals. Soaked crystals were fished after overnight incubation at 4° C. and flash-cooled in liquid nitrogen.

X-ray diffraction (XRD) data were collected either in-house on a Rigaku Compact HomeLab (equipped with Rigaku MicroMax-003 sealed tube X-ray source and Rigaku Dectris PILATUS3 R 200K detector; 1433σC45/ERα/3), at the Deutsches Elektronen-Synchrotron (DESY) PETRA-III beamline P11, Hamburg, Germany (14336C45/ERα/4 and 5; and all 14336C42/ERα datasets).

Initial processing of all datasets was done using Pipedream from GlobalPhasing. First, Autoproc ran XDS for data indexing and integration, and AIMLESS for scaling. The structures were phased by limited molecular replacement, using protein data bank (PDB) entry 4JC3 (ERα) as a template, in Phaser. Finally, Buster was used for initial structure refinement. Upon completion of the Pipedream run, the presence of soaked fragments was verified by visual inspection of the Fo-Fc and 2Fo-Fc electron density maps in Coot. Structure and restraints were generated using eLBOW or grade for successfully soaked ligands before using phenix.refine and Coot in alternating cycles for model building and refinement.

Crystallographic data were deposited in the Protein Data Bank (PDB) and obtained accession codes 7B9M, 7B9R, 7B9T, 7BA3, 7BA5, 7BA6, 7BA7, 7BA8, 7BA9, 7BAA, and 7BAB.

Synthesis of Derivatives for C42-Tethered Fragments

General Remarks. Unless otherwise stated, all solvents employed were commercially available and used without purification. Deuterated solvents were obtained from Cambridge Isotope Laboratories. Water was purified using a Millipore purification train. Dry solvents were obtained from a MBRAUN Solvent Purification System (MB-SPS-800). All reagents were obtained from Sigma Aldrich and used without purification. Reaction progress was monitored by analytical thin-layer chromatography (TLC, pre-coated silica gel 60 F254 plates, Merck) using ultraviolet (UV) light (254 and 365 nm). Analytical liquid chromatography coupled with mass spectrometry (LC-MS) was performed on a C4 Jupiter SuC4300A 150×2.0 mm column (using a 15 min. gradient of 5% to 100% acetonitrile in H₂O (0.1% formic acid)), connected to a ThermoFischer LCQ Fleet Ion Trap Mass Spectrometer. Preparative high-pressure column chromatography was performed on a Grace™ Reveleris™ system using SRC C18 cartridges. NMR data were recorded on a Bruker Advance-III 400 MHz equipped with a BBFO probe from Bruker (400 MHz for ¹H-NMR and 100 MHz for ¹³C-NMR). Chemical shifts were reported in parts per million (ppm) referenced to an internal standard (d-chloroform; 7.26 ppm for ¹H-NMR and 77 ppm for ¹³C-NMR), relative to tetramethylsilane (TMS). ¹H-NMR and ¹³C-NMR signals were assigned with the aid of two-dimensional ¹H, ¹H-COSY, and ¹H, ¹³C-HSQC spectra.

Synthetic Procedure

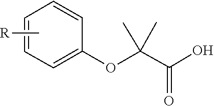

2-methyl-2-phenoxypropanoic acid derivatives. To each phenol derivative (1 mmol) dissolved in DMF (2 mL) was added tert-butyl-2-bromo-2-methylpropanoate (3 mmol), K₂CO₃ (4 mmol) and MgSO₄ (1 mmol). Mixtures were heated to 100° C. and stirred overnight under argon atmosphere. After cooling, reaction mixtures were extracted with EtOAc, washed with water 3× and brine. The organic layers were dried over MgSO₄ and concentrated in vacuo. The crudes were purified by automated column chromatography (C18, Grace™ Reveleris™ system, heptane/EtOAc 0-20%). Purity was verified by gas chromatography-mass spectrometry (GCMS) before proceeding. Deprotection of the acids was performed in a solution of 1:1 DCM/TFA (3 mL), which was stirred at room temperature for 5 hours. Solvent was removed in vacuo to yield the pure 2-methyl-2-phenoxypropanoic acid derivatives (40-60%). Completion of deprotection was confirmed by GCMS for all derivatives.

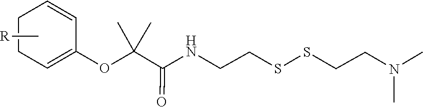

N-(2-((2-(dimethylamino)ethyl)disulfaneyl)ethyl)-2-methyl-2-phenoxypropanamide derivatives (6-13). The 2-methyl-2-phenoxypropanoic acid derivatives (0.4 mmol) were each dissolved in a mixture of DMF, THF and water (4 mL, 5:4:1 v/v). To this was added cystamine (2,2′-disulfanediylbis(ethan-1-amine), 0.21 mmol), HBTU (1.2 mmol) and TEA (2.0 mmol). The reaction was stirred at room temperature overnight. Upon completion, which was verified with LCMS, tris(2-carboxyethyl)phosphine hydrochloride (TCEP, 0.04 mmol) and 2,2′-disulfanediylbis(N—N-dimethylethan-1-amine) were added to the reaction mixture to initiate disulfide exchange. After stirring at room temperature overnight, the solvent was removed in vacuo. Products were purified by column chromatography (C18, Grace™ Reveleris™ system, heptane/DCM 10-70%, containing 4% TEA), yielding final compounds 6-13 (25-32%). 6: ¹H NMR (400 MHz, Chloroform-d) δ 7.01-6.93 (m, 2H), 6.93-6.87 (m, 2H), 3.66 (q, J=6.1 Hz, 2H), 2.89-2.77 (m, 4H), 2.59 (t, J=8.1, 6.2 Hz, 2H), 2.25 (s, 6H), 1.47 (s, 6H). ¹³C NMR (100 MHz, CDCl₃) δ 174.73, 153.53, 132.39, 123.11, 116.13, 81.95, 58.54, 45.20, 38.28, 37.91, 25.16. 7: ¹H NMR (400 MHz, Chloroform-d) δ 7.01-6.93 (m, 2H), 6.93-6.87 (m, 2H), 3.66 (q, J=6.1 Hz, 2H), 2.89-2.77 (m, 4H), 2.59 (t, J=8.1, 6.2 Hz, 2H), 2.25 (s, 6H), 1.47 (s, 6H). ¹³C NMR (100 MHz, CDCl₃) δ 174.78, 160.17, 157.77, 123.22, 123.13, 115.88, 115.65, 81.92, 58.62, 45.35, 38.08, 37.79, 36.91, 24.94. 8: ¹H NMR (400 MHz, Chloroform-d) δ 6.53 (tt, J=8.9, 2.3 Hz, 1H), 6.48-6.43 (m, 2H), 3.62 (q, J=6.2 Hz, 2H), 3.08-2.98 (m, 2H), 2.98-2.91 (m, 2H), 2.87 (t, J=6.3 Hz, 2H), 2.62 (s, 6H), 1.55 (s, 6H). ¹³C NMR (100 MHz, CDCl₃) δ 174.26, 164.60, 162.14, 156.53, 105.02, 98.84, 81.99, 57.81, 44.08, 38.43, 38.02, 33.34, 25.16. 9: ¹H NMR (400 MHz, Chloroform-d) δ 7.08 (t, J=1.8 Hz, 1H), 6.84 (s, 1H), 6.83 (s, 1H), 3.66 (q, J=6.1 Hz, 2H), 2.87-2.76 (m, 4H), 2.58 (t, J=8.0, 6.2 Hz, 2H), 2.25 (s, 6H), 1.54 (s, 6H). ¹³C NMR (100 MHz, CDCl₃) δ 173.86, 155.49, 135.14, 123.59, 119.70, 82.57, 58.58, 45.34, 38.12, 37.61, 36.88, 25.04. 10: ¹H NMR (400 MHz, Chloroform-d) δ 7.31-7.26 (m, 2H), 7.10-7.04 (m, 1H), 6.95-6.91 (m, 2H), 3.65 (q, J=6.2 Hz, 2H), 2.86-2.76 (m, 4H), 2.58 (t, J=8.1, 6.2 Hz, 2H), 2.25 (s, 6H), 1.51 (s, 6H). ¹³C NMR (100 MHz, CDCl₃) δ 175.02, 154.21, 129.25, 123.31, 121.35, 81.45, 58.61, 45.34, 38.13, 37.77, 36.87, 25.11. 11: ¹H NMR (400 MHz, Chloroform-d) δ 7.11-7.02 (m, 2H), 6.86-6.79 (m, 2H), 3.65 (q, J=6.2 Hz, 2H), 2.88-2.76 (m, 4H), 2.58 (t, J=8.1, 6.2 Hz, 2H), 2.30 (s, 3H), 2.25 (s, 6H), 1.48 (s, 6H). ¹³C NMR (100 MHz, CDCl₃) δ 175.18, 151.82, 132.93, 129.72, 121.49, 81.40, 58.63, 45.35, 38.14, 38.10, 37.81, 36.91, 25.04, 20.65. 12: ¹H NMR (400 MHz, Chloroform-d) δ 7.39 (d, J=8.6, 1H), 6.53-6.49 (m, 1H), 6.43-6.36 (m, 1H), 3.86 (s, 3H), 3.65 (q, J=6.2 Hz, 2H), 2.86-2.76 (m, 4H), 2.60 (t, J=8.1, 6.2 Hz, 2H), 2.27 (s, 6H), 1.53 (s, 6H). LCMS (ESI) calc. for (6) C₁₆H₂₅BrN₂O₂S₂ [M] 420.05; observed [M+H]+ 421.08, LC Rₜ=5.76 min. LCMS (ESI) calc. for (7) C₁₆H₂₅FN₂O₂S₂ [M] 360.13; observed [M+H]+ 361.08, LC Rₜ=3.80 min. LCMS (ESI) calc. for (8) C₁₆H2₄F2N₂O₂S₂ [M] 378.12; observed [M+H]+ 379.00, LC Rₜ=4.17 min. LCMS (ESI) calc. for (9) C₁₆H2₄C1₂N₂O₂S₂[M] 410.07; observed [M+H]+ 411.08, LC Rₜ=4.47 min. LCMS (ESI) calc. for (10) C₁₆H2₆N₂O₂S₂[M] 342.14; observed [M+H]+ 343.08, LC Rₜ=3.84 min. LCMS (ESI) calc. for (11) C₁₇H2₈N₂O₂S₂[M] 356.16; observed [M+H]+ 357.08, LC Rₜ=4.06 min. LCMS (ESI) calc. for (12) C₁₇H₂₇BrN₂O₃S₂ [M]452.06; observed [M+H]+ 453.00, LC Rₜ=4.08 min. LCMS (ESI) calc. for (13) C₁₆H₂₄ClN₃O₄S₂ [M] 421.09; observed [M+H]+ 422.00, LC Rₜ=3.96 min.

### Example 9: Selectivity in the 14-3-3 Hub Protein Interactions Via Reversible Covalent (PPI) Stabilizers

We selected the interaction between 14-3-3 and the Peptidyl-prolyl cis-trans isomerase NIMA-interacting 1 (Pin1) which is closely involved in many disease states as a relevant case study. The formation of the 14-3-3/Pin1/Myc complex is reported to drive the ubiquitination and pro-teasomal degradation of oncogenic Myc by ubiquitin ligase Fbxw7.

We show that the unique topologies and functionalities of various binding interfaces shaped by the complexes of 14-3-3 will direct for specific molecular fragments that selectively stabilize a specific 14-3-3 PPI. Utilizing an imine-tethering approach, we demonstrate how selectivity can be engineered in the early stages of the drug discovery process. We exploit a privileged anchor point of Lys122 that lies at the interface of the composite binding pocket formed by the protein complex. This binding pocket is situated adjacent to the phospho-accepting pocket. Fragment binding is only compatible with bent partner protein epitopes. Further selectivity is driven by templating effects of the amino acid in the plus one position relative to the phosphorylated residue of the interaction partner.

Elucidation of 14-3-3/Pin1 interaction. We have developed an aldehyde fragment screening approach, targeting the p65/14-3-3σ PPI. This site-directed fragment screening approach forms an aldimine bond between the aldehyde fragment and Lys122 of 14-3-3σ. Lysine present an attractive anchoring point for covalent drug discovery owing to the large representation of lysine in the proteome, Lys122 is located within the binding groove of 14-3-3, adjacent to the p65/14-3-3 interface. This privileged location of imine bond formation offers the unique opportunity to evaluate the efficiency and selectivity of aldehydes stabilizing complex formation with the hub protein 14-3-3. Research by Wen et al. has suggested that 14-3-3 binds the Pin1 protein in a disordered loop region (Val62-Thr81). Further screening of the protein sequence with a 14-3-3 prediction server40 further supported the proposed binding site being within the loop region of Pin1. Amino acids Ser71 and Ser72 were identified as potential 14-3-3 recognition sites. Computational screening predicted that the pSer72 site was the more likely binding motif (Table 5). Considering the proximity of the two amino acids in the binding motif, we tested both phosphorylation sites. We screened 17-mer phosphopeptides representing the loop region of Pin1 whereby either Ser71 or Ser72 were phosphorylated. The elucidation of binding affinities was done using a fluorescence anisotropy (FA) assay with 14-3-3γ. The pSer72 (Pin1_72) peptide elicited a K_{D} of 22.2±1.20 μM. In contrast, a K_{D} of ˜270 μM was observed for the pSer71 peptide. Next, the Pin1_72 peptide was crystallized in complex with 14-3-3σ, at 1.5 Å resolution. Notably, we were unable to crystallize the pSer71 site. Analysis of the complex showed that Pin1_72 occupied two-third of the amphiphilic phospho-binding groove of 14-3-3. Of particular interest was the orientation of Trp73 of Pin1_72 due to its hydrophobic interactions with the 14-3-3 surface. Further, the C-terminus of the peptide veers out of the binding groove, generating a composite pocket for small molecule binding.

TABLE 5
The 14-3-3pred server allows the in silica
analysis of potential 14-3-3 binding
sites of Pin1.

| Posi- | Peptide | | | | |
|---|---|---|---|---|---|
| tion | [−6:4] | ANN | PSSM | SVM | Consensus |
| 18 | EKRMSR[S] | 0.655 | 0.376 | −0.187 | 0.281 |
| | SGRV | | | | |
| | (SEQ ID | | | | |
| | NO: 15) | | | | |
| 19 | KRMSRS[S] | 0.236 | 0.172 | −0.741 | −0.111 |
| | GRVY | | | | |
| | (SEQ ID | | | | |
| | NO: 16) | | | | |
| 29 | YYFNHI[T] | 0.112 | −0.118 | −1.135 | −0.380 |
| | NASQ | | | | |
| | (SEQ ID | | | | |
| | NO: 17) | | | | |
| 32 | NHITNA[S] | 0.344 | 0.017 | −1.148 | −0.229 |
| | QWER | | | | |
| | (SEQ ID | | | | |
| | NO: 18) | | | | |
| 38 | SQWERP[S] | 0.049 | −0.032 | −1.242 | −0.408 |
| | GNSS | | | | |
| | (SEQ ID | | | | |
| | NO: 19) | | | | |
| 41 | ERPSGN[S] | 0.062 | −0.107 | −1.383 | −0.476 |
| | SSGG | | | | |
| | (SEQ ID | | | | |
| | NO: 20) | | | | |
| 42 | RPSGNS[S] | 0.136 | −0.226 | −0.929 | −0.340 |
| | SGGK | | | | |
| | (SEQ ID | | | | |
| | NO: 21) | | | | |
| 43 | PSGNSS[S] | 0.147 | 0.033 | −0.636 | −0.152 |
| | GGKN | | | | |
| | (SEQ ID | | | | |
| | NO: 22) | | | | |
| 58 | PARVRC[S] | 0.525 | 0.332 | −0.614 | 0.081 |
| | HLLV | | | | |
| | (SEQ ID | | | | |
| | NO: 23) | | | | |
| 65 | HLLVKH[S] | 0.142 | −0.013 | −1.085 | −0.318 |
| | QSRR | | | | |
| | (SEQ ID | | | | |
| | NO: 24) | | | | |
| 67 | LVKHSQ[S] | 0.214 | −0.020 | −0.469 | −0.092 |
| | RRPS | | | | |
| | (SEQ ID | | | | |
| | NO: 25) | | | | |
| 71 | SQSRRP[S] | 0.552 | 0.712 | −0.128 | 0.379 |
| | SWRQ | | | | |
| | (SEQ ID | | | | |
| | NO: 26) | | | | |
| 72 | QSRRPS[S] | 0.583 | 0.821 | 0.370 | 0.591 |
| | WRQE | | | | |
| | (SEQ ID | | | | |
| | NO: 27) | | | | |
| 79 | WRQEKI[T] | | | | |
| | RTKE | 0.118 | −0.035 | −1.168 | −0.362 |
| | (SEQ ID | | | | |
| | NO: 28) | | | | |
| 81 | QEKITR[T] | | | | |
| | KEEA | 0.114 | −0.035 | −1.036 | −0.319 |
| | (SEQ ID | | | | |
| | NO: 29) | | | | |
| 98 | YIQKIK[S] | 0.519 | 0.214 | 0.162 | 0.298 |
| | GEED | | | | |
| | (SEQ ID | | | | |
| | NO: 30) | | | | |
| 105 | GEEDFE[S] | 0.159 | −0.151 | −1.308 | −0.433 |
| | LASQ | | | | |
| | (SEQ ID | | | | |
| | NO: 31) | | | | |
| 108 | DFESLA[S] | 0.167 | −0.173 | −1.130 | −0.379 |
| | QFSD | | | | |
| | (SEQ ID | | | | |
| | NO: 32) | | | | |
| 111 | SLASQF[S] | 0.062 | −0.163 | −1.479 | −0.527 |
| | DCSS | | | | |
| | (SEQ ID | | | | |
| | NO: 33) | | | | |
| 114 | SQFSDC[S] | 0.069 | −0.328 | −1.607 | −0.622 |
| | SAKA | | | | |
| | (SEQ ID | | | | |
| | NO: 34) | | | | |
| 115 | QFSDCS[S] | 0.248 | 0.007 | −0.605 | −0.117 |
| | AKAR | | | | |
| | (SEQ ID | | | | |
| | NO: 35) | | | | |
| 126 | GDLGAF[S] | 0.262 | −0.048 | −0.479 | −0.088 |
| | RGQM | | | | |
| | (SEQ ID | | | | |
| | NO: 36) | | | | |
| 138 | KPFEDA[S] | 0.152 | −0.078 | −1.023 | −0.316 |
| | FALR | | | | |
| | (SEQ ID | | | | |
| | NO: 37) | | | | |
| 143 | ASFALR[T] | 0.050 | −0.369 | −1.800 | −0.706 |
| | GEMS | | | | |
| | (SEQ ID | | | | |
| | NO: 38) | | | | |
| 147 | LRTGEM[S] | 0.528 | 0.730 | 0.209 | 0.489 |
| | GPVF | | | | |
| | (SEQ ID | | | | |
| | NO: 39) | | | | |
| 152 | MSGPVF[T] | 0.200 | −0.067 | −0.747 | −0.205 |
| | DSGI | | | | |
| | (SEQ ID | | | | |
| | NO: 40) | | | | |
| 154 | GPVFTD[S] | 0.131 | −0.120 | −0.970 | −0.320 |
| | GIHI | | | | |
| | (SEQ ID | | | | |
| | NO: 41) | | | | |
| 162 | IHIILR[T] | 0.118 | 0.148 | −1.347 | −0.360 |
| | E | | | | |
| | (SEQ ID | | | | |
| | NO: 42) | | | | |

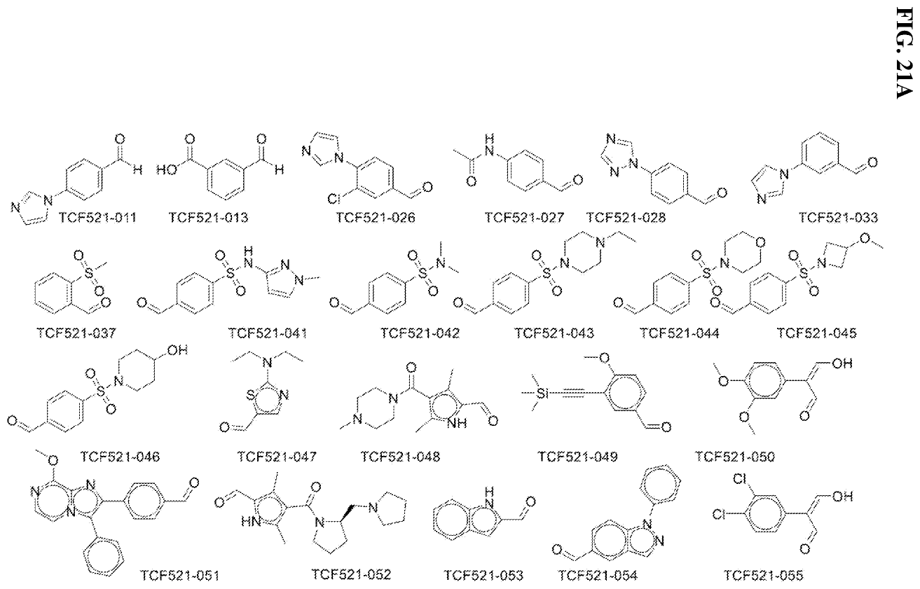

Site-Directed Shift base fragment Screening. Given the solvent exposure of Lys122 of 14-3-3 in the complex with Pin1_72, we selected 42 covalent fragments from an in-house aldehyde fragment library for fragment screening with the Pin1_72/14-3-3σ complex using a FA assay (FIGS. 21A-21B). Critical to this selection was the knowledge that several fragments bound in the p65/14-3-3σ complex, observed by X-ray, although did not elicit a stabilizing effect in FA assays, herein termed silent binders. Fragments were screened by titration to a fixed concentration of 14-3-3σ (10 μM) and Pin1_72 (50 nM). As measure of activity the inflection point of the curve was determined, representing the half-maximum complex formation (CC₅₀). From the fragment screen, 11 compounds were found to stabilize the Pin1_72/14-3-3σ complex. Of these fragments, L2 and L3 were shown to exhibit significant complex formation, albeit that a lack of upper plateau limited accurate assignment of the CC₅₀ values. Notably, fragment L1, which did not contain a halogen was not active. Inquisitive regarding the binding of L1, we also soaked this fragment with the 14-3-3σΔC/Pin1_72 complex. X-ray crystal structures of L1, L2 and L3 in complex with 14-3-3σΔC/Pin1_72 confirmed that all fragments formed a covalent imine bond with Lys122. The binding of all induced a conformational change in Pin1_72 when compared with the binary complex. Specifically, Trp73 of Pin1, herein denoted Trp+1, describing its position relative to the phosphorylated Ser72, was flipped ˜90° forming a t-t interaction with the fragment.

Fragment extension and SAR analysis. Having identified L2 and L3 as hit fragments for optimization, we sought to extend the fragments, with a focus on enhancing potency for the Pin1_72/14-3-3 complex. Three key sub-pockets were identified (P1, P2 and P3) as potential points for fragment extension. A focused library was constructed utilizing a nucleophilic aromatic substitution reaction, with substituted imidazoles or benzoimidazole and substituted 4-fluorobenzaldehydes (Table 6). Initial library development focused on halogen substitution and shifting the position of the halogen to probe pocket P1. Further, we investigated the effect of substituted imidazoles to explore pockets P2 and P3. Analysis using the FA assay showed an exchange of the chloride of L2 for bromine (1) resulted in a loss of activity. In contrast, 2-substituted chlorine (2) and bromine analogues (3) resulted in improved affinity to the complex, with CC₅₀ of 200±27.0 μM and 136±99.0 μM, respectively (Table 6). Decorations on the imid-azole ring (4-9) did induce minor to no complex stabilization.

TABLE 6
Structural analogues of T1 and T2 were designed to explore the composite binding pocket of the
Pin1/14-3-3 complex.

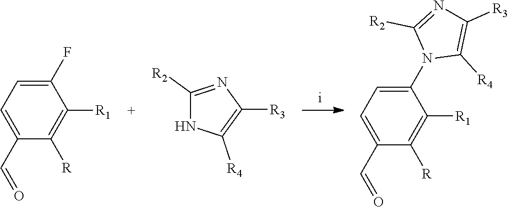

| Com- | | | | | | | App. KD | | |
|---|---|---|---|---|---|---|---|---|---|
| pound | R | R1 | R2 | R3 | R4 | CC₅₀ (μM) | (μM) ^{b} | SF^{c} | PDB |
| DMSO | — | | — | — | | — | 22.2 ± 1.23 | — | 7AOG |
| L1 | H | H | H | H | H | >1000 | | | 7NIF |
| L2 | H | Cl | H | H | H | 423 ± 130 | 24.01 | 0.8 | 7AXN |
| L3^{a} | H | H | Br | H | H | 480 ± 74.0 | 14.87 | 1.3 | 7AYF |
| 1 | H | Br | H | H | H | >1000 | | | n.d. |
| 2 | Cl | H | H | H | H | 200 ± 27.0 | 9.09 | 2.1 | n.d. |
| 3 | Br | H | H | H | H | 136 ± 99 | 6.34 | 3.0 | 7NIG |
| 4 | H | H | H | Me | H | >1000 | | | 7NRK |
| 5 | H | H | H | H | Me | >1000 | | | n.d. |
| 6 | H | H | H | CF₃ | H | >1000 | | | 7NJ6 |
| 7 | H | H | H | Benzyl | | >1000 | | | 7NJ8 |
| 8 | H | H | COOH | H | H | >1000 | | | n.d. |
| 9 | H | H | Phenyl | H | H | >1000 | | | 7NJA |
| 10 | Cl | H | Phenyl | H | H | 166 ± 30.9 | 5.37 ± 0.30 | 3.6 | 7BDP |
| 11 | H | Cl | Phenyl | H | H | >500 | | | n.d. |
| 12 | H | Br | Phenyl | H | H | >500 | | | n.d. |
| 13 | Br | H | Phenyl | H | H | 101 ± 5.88 | 1.67 ± 0.04 | 15.6 | 7BDT |
| 14 | CF₃ | H | Phenyl | H | H | 306 ± 42.3 | 15.6 ± 1.29 | 1.2 | 7AZ1 |
| 15 | H | CF₃ | Phenyl | H | H | >500 | | | 7AZ2 |
| 16 | OMe | H | Phenyl | H | H | -d | | | 7BGQ |
| 17 | H | OMe | Phenyl | H | H | -d | | | 7BGV |
| 18 | Me | H | Phenyl | H | H | -d | | | 7BGR |
| 19 | OH | H | Phenyl | H | H | 19.2 ± 14.6 | 3.92 ± 0.25 | 4.9 | 7NRL |
| 20 | OCF₃ | H | Phenyl | H | H | >500 | | | n. bind. |
| 21 | OPh | H | Phenyl | H | H | -d | | | n. bind. |
| 22 | Naphth | | Phenyl | H | H | -d | | | 7BGW |
| 23 | Br | H | 2-Br | H | H | 23.9 ± 3.22 | 1.15 ± 0.07 | 18.9 | 7BG3 |
| | | | phenyl | | | | | | |
| 24 | Br | H | 4-Br | H | H | 105.9 ± 17.0 | 5.77 ± 1.01 | 3.32 | n. bind |
| | | | phenyl | | | | | | |
| 25 | Br | H | 4-OH | H | H | >500 | | | n.d. |
| | | | phenyl | | | | | | |
| 26 | Br | H | 3- | H | H | 92.2 ± 8.42 | 8.47 ± 0.64 | 2.27 | n.d. |
| | | | pyridinyl | | | | | | |
| 27 | Br | H | 2-F, 5-Br | H | H | 117 ± 6.50 | 0.771 ± 0.02 | 33.5 | 7BDY |
| | | | phenyl | | | | | | |
| 28 | Br | H | 2,4-diF | H | H | 78.8 ± 2.76 | 0.293 ± 0.01 | 93.0 | 7BFW |
| | | | phenyl | | | | | | |

^{a}contains a nicotinaldehyde scaffold; ^{b}Measurements were taken after overnight incubation and in presence of 100 μM fragment; ^{c}Fold stabilization was measured based on the internal DMSO control and 100 μM fragment; ^{d}Compound showed autofluorescence within the FP assay; n.d.: not determined; n. bind.: no extra electron density due to compound binding.

Analysis of X-ray crystal structures of fragments provided valuable insight into the activity profile of this library of compounds. All measured fragments, 1-9, bound to Lys122, notably, 4-9 proved to be silent binders. They induced a similar shift of the Trp+1 residue of Pin1_72, forming a 7-7 stack between the indole side chain and the benzaldehyde ring of the fragment. Further, shift of the halogen to the 2-position probes the P1 sub-pocket formed by residues Asn42, Val46, Phe119 and Lys122 of 14-3-3. Fragments 4, 5 and 7 probe the P3 pocked comprised of Asp215, Leu218, Ile219 and Leu222. The occupancy of the electron density map for fragments 4, 5 and 7 is low and prevents accurate positioning of the imidazole decorations. However, the tri-fluoro of 6 reaches Asp215 of 14-3-3 and the benzimidazole of 7 engages in hydrophobic contacts with Leu218 and Ile219 in the roof of 14-3-3. Lastly, the installation of a phenyl ring in the 2-position of the imidazole ring (9) probed sub-pocket P2 formed by Ile168, Asn42, and Phe119.

Inspired by the binding poses of 3 and 9 we combined their structural features to improve stabilization. Synthesis and screening of compounds 10-22 identified that a 2-bromo (13) or 2-hydroxy (19) substituted phenyl imidazole provided CC₅₀ values of 101±5.88 and 23.9±3.22 μM, respectively (Table 6). The CC₅₀ values were further confirmed by protein titration assay using FA in presence of a constant concentration of compound (100 μM). In case of complex stabilization, a left shift of apparent K_{D} values is expected, here described as stabilization factors (SF). Protein titration assay showed that fragment 13 (app. K_{D}=2.85±0.09 μM, SF=10.8) elicited improved stabilization of the ternary complex formation relative to 19 (app K_{D}=3.92±0.25 μM, SF=4.9).

Analysis of the ternary crystal structure showed that 13 bound in a similar conformation to fragment 3 and 9. Interestingly, a conformational change is observed in Asn42 of 14-3-3 and the C-terminus of the Pin1_72. This induces a water mediated hydrogen bond interaction between Gln+3 of Pin1_72 and Asn42. This conformational change is highly advantageous as this enhances the polar contact between Pin1 and 14-3-3. Inspection of the electron density mesh of 13 suggested that its 2-phenyl freely rotated. Further, Asn42 occupied two different conformations indicating either a low occupancy of the fragment or a high conformational freedom. We therefore investigated introduction of bulky side groups and/or hydrogen bonding groups to the 2-phenyl imidazole to impair free rotation (23-28, Table 6). Introduction of a hydrogen bonding group proved to have limited effect (25 and 26) with 26 only showing weak stabilization (SF=2.27). Increasing the bulk of the 2-phenyl ring proved highly effective in improving potency and stabilization with 2-bromo (23), 2-fluoro-5-bromo (27) and 2,4-difluoro (28) eliciting CC₅₀ values of 23.9±3.22, 117.5±6.50 μM and 78.8±2.76, respectively (Table 6). Further, 23, 27 and 28 showed a significant shift in apparent K_{D} ranging from single-digit micromolar to sub-micromolar activity (1.95-0.28 μM). This translated to SFs ranging from 13-93-fold stabilization.

To benchmark the activity of fragment 28, we also screened known 14-3-3 stabilizer Fusicoccin A (FCA) against Pin1. FCA preferentially stabilizes 14-3-3 interaction partners with C-terminal phosphorylation sites (pSer/pThr-X-COOH, X: hydrophobic residue), like those present in the estrogen receptor a (ERα). Protein titrations with FCA afforded an apparent K_{D} of 3.32 0.25 M, an order of magnitude less potent than 28.

Cooperativity in ternary complex formation. In contrast to PPI inhibition, where affinity to one of the protein pockets is the driving force for drug development, design of molecular glues is driven by cooperative ternary complex formation. Both CC₅₀ and SF values are concentration dependent values and might differ based on assay design. Hence, we were aiming to determine the cooperativity factor (α) as concentration independent measure of cooperativity. Cooperative complex formation is often accompanied by structural changes of the interface of a complex which translates to increased stability of the ternary com-plex. In order to assess cooperativity of the ternary complex, fragments 13, 23, 27 and 28 were selected for cooper-activity analysis. The α-factor of the fragments were determined using 14-3-3 titrations in the presence of varied, but constant concentration of fragment in a dose-dependent manner. The α-factor also describes the SF of a saturated system, where higher compound concentrations do not further decrease the apparent K_{D}. Further, the interval of change in stabilization further describes the systems cooperative behaviour.

Cooperativity analysis of 13 showed that the compound induced an order of magnitude decrease of the app. K_{D} of the 14-3-3/Pin_72 complex at 250 μM. However, at higher concentration regimes significant assay interference was observed, probably due to compound aggregation. Fragments 23, 27 and 28 all reached saturation or approached saturation enabling accurate determination of α-factor. Fragments 13, 23 and 27 showed α-factors of approximately 60. Notably, 27 reached saturation at significantly lower concentration, compared to 23, resulting in the previously observed difference of SFs. The 14-3-3/Pin1/28 complex showed the highest cooperativity with an α-factor=270 and with only 1 μM of 28 necessary to induce already a 2-fold increase in PPI stabilization. Interestingly, whilst FCA elicits significant stabilization for the 14-3-3/Pin1 complex at concentrations of 8 μM (SF 8 μM=˜10), at a concentration of 100 μM, the observed shift of app. K_{D} remains constant also at higher concentrations of FC-A, indicating non-specific effects. This cooperative profile may be a function of the bulky hydrophobic properties of FCA, having a higher intrinsic affinity to 14-33 but being less compatible with the size of Trp+1 for optimal stabilization.

In order to better understand how structural changes in 13, 23, 27 and 28 translated to different cooperativity, the compounds were soaked into 14-3-3/Pin1 crystals. Analysis of the crystal structures shows conformational changes at the composite interface that potentially drive cooperative behavior. The 14-3-3/Pin1/28 complex showed a conformational change in Asn42 side chain of 14-3-3 induced by the presence of the 2,4-difluorophenyl ring of 28. Specifically, this induces a conformational change in Asn42 of 14-3-3 facilitating a direct hydrogen bond with Gln+3 of Pin1. Notably, this interaction is absent in the crystal structures of 13 and 27. Additionally, we observed that the 4-fluoro occupies a deep pocket formed by Cys38, Arg41 and Phe119, thereby locking the orientation of the 2,4-difluorophenyl ring. It was also observed that the indole side chain of Trp+1 has an inverted conformation compared to 13 and 27. Notably, the 14-3-3/Pin1/23 complex shows two conformations for Trp+1 suggesting that the side chain is not in the lowest energy state. Furthermore, the alternative Trp+1 conformation induced by 28 allows formation of water mediated hydrogen bonds between the indole moiety of Trp+1 and Gln+3 of Pin1 and Asn42 and Ser45 of 14-3-3. These additional contacts at the interface of the complex potentially explain the improved cooperative behavior. We further hypothesize that these Pin1 specific interactions will result in high selectivity of these fragments towards the Pin1/14-3-3 complex.

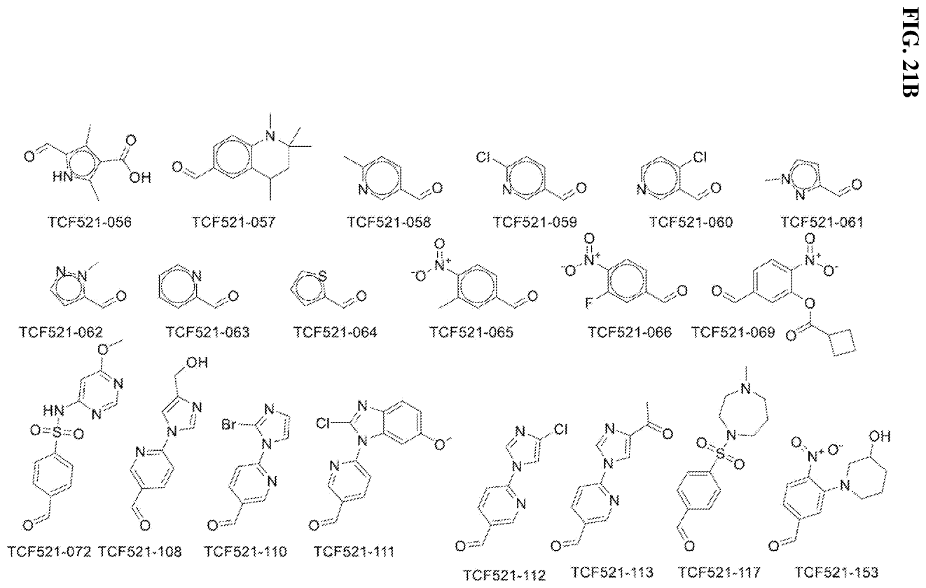

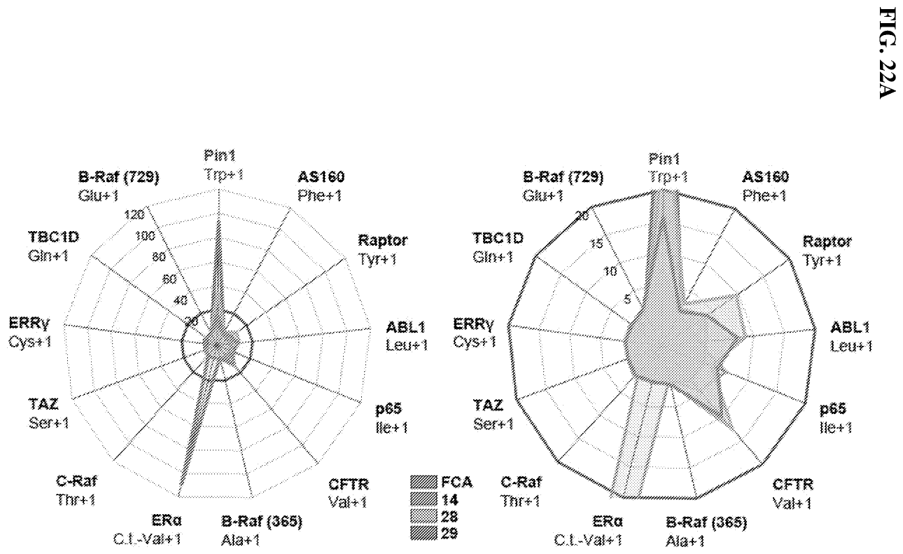

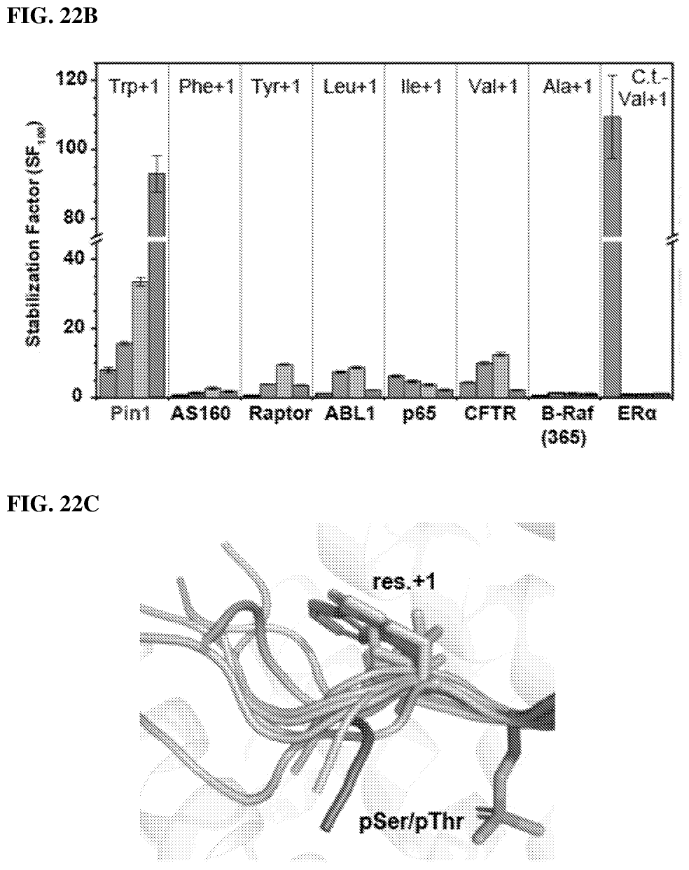

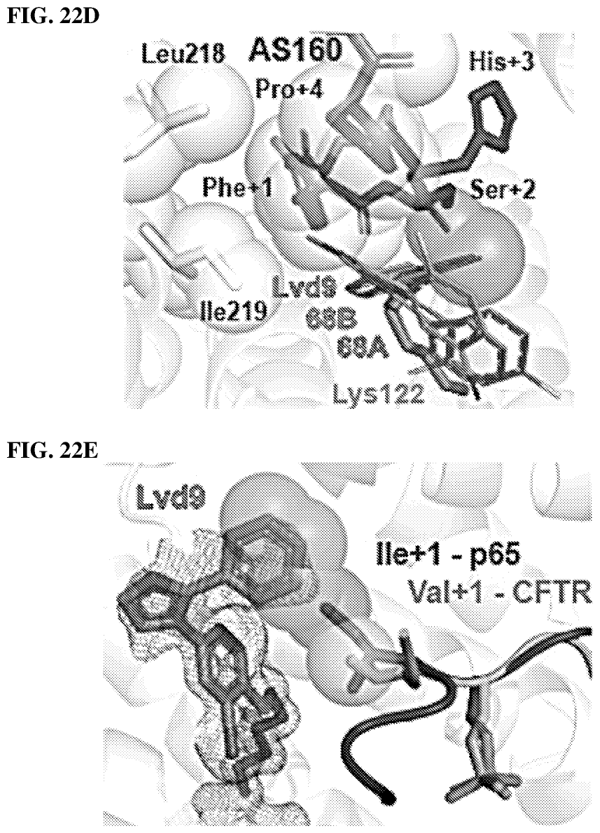

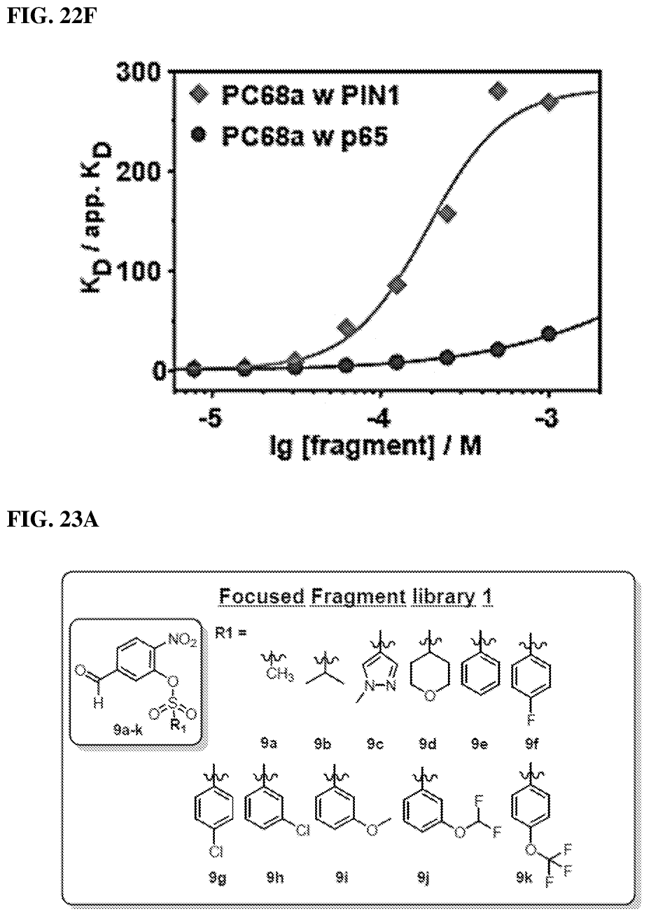

Selectivity screening of covalent fragments. Drugging the hub protein 14-3-3 raises the challenge of selectivity. We hypothesized that the high level of cooperative behavior for 14-3-3/Pin1_72/28 complex is a function of the unique functionality and topology of the interface, specifically the +1 and +3 amino acid of Pin1_72 with the covalent fragment. We further rationalized that this cooperativity would likely translate to high selectivity. To test this hypothesis, fragments 13, 27 and 28 were screened at a single fragment concentration (100 μM) against a panel of 13 peptides as diverse representatives of 14-3-3 client proteins, differing in size and hydrophobicity of the +1 amino acid (FIG. 22A).

First, 14-3-3 interaction partners with polar amino acids in the +1 position were investigated. C-Raf has a threonine in the +1 position, whereby the hydroxyl group sufficiently abolishes any stabilizing effect of 13, 27 and 28. Glutamic acid, glutamine, cysteine or serine, in this position, as offered by the B-Raf_729, TBC1D237, ERRγ_179 and Mypt1_472 peptides, showed no significant stabilization with 13, 27 and 28. A polar amino acid in the +1 position is not compatible with these imine forming fragments. This is likely due to the direct hydrogen bond possible between Lys122 of 14-3-3 and the polar side chain of the +1 amino acid, coupled with repulsive behavior of a polar amino acid perpendicular to the aromatic ring of benzaldehyde. Similarly to polar +1 amino acids, a C-terminal phosphorylation motif, as prototypical for ERα was also not responsive to fragment stabilization with 13, 27 and 28. Again, salt bridge formation between Lys122 and the carboxylic acid terminus of ERα is the most logical rationale. This is in contrast to the natural compound FCA which elicits a 110-fold stabilization of the 14-3-3/ERα complex. Remarkably, none of the fragments had any significant inhibiting effects on binary complex formation, indicating a very low intrinsic affinity of the aldehyde fragments towards 14-3-3 alone. This leads to a desirable, non-competitive binding mode.

Following the importance of the tryptophan for complex stabilization of Pin1_72/14-3-3γ with the benzaldehydes, the influence of phenylalanine (AS160) and tyrosine (Raptor) were investigated (FIGS. 22A-22B). No appreciable stabilization of the AS160/14-3-3γ complex was observed with any of the fragments (13, 27 and 28), with SFs ranging from 1.2-2.7. The crystal structure of AS160 shows that the phenyl side chain employs similar hydrophobic contacts with the roof of 14-3-3 as Trp+1 of Pin1_72 (FIG. 22D). Unlike Pin1_72, the C-terminus of AS160 engages Phe+1 in intramolecular hydrophobic contacts with Pro+4 and Pro+5. The +1 phenylalanine likely cannot rearrange to allow fragment binding. The Raptor/14-3-3γ binary complex proved to be more responsive to fragment stabilization with 27 showing a 9.5-fold stabilization of the binary complex.

The aldimine formation with Lys122 was first identified for the p65/14-3-3 interaction, with p65 containing an isoleucine at the +1 position. Hence, small hydrophobic residues could potentially form hydrophobic contacts with the benzaldehyde scaffold. This was investigated by comparing the effect of 13, 27 and 28 on 14-3-3 interaction partners with a leucine (Abl1pT735), isoleucine (p65pS45), or valine (CFTRpS753) at the +1 position. Fragments 13 and 27 elicited some stabilizing activity for all three interaction partners with SF values ranging from 4.7 for 13 with p65 to 12.5 for 27 with CFTR. Fragment 28 induced no significant complex stabilization. The B-Raf_365 peptide with an alanine in the +1 position was not responsive to complex stabilization by any of the imine-forming aldehydes. This is likely a result of the topology formed by the C-terminus of B-Raf_365, which creates a smaller binding pocket occluding the fragments.

Soakings of 13 and 23 into p65/14-3-3σΔC complexes provided an explanation of selectivity. The ternary complex with p65/14-3-3σΔC/13 showed a distinct binding pose to the fragments in comparison with Pin1_72/14-3-3σΔC. Specifically, the 2-phenyl ring of 13 and 23 points towards Ile+1 of p65_45 (FIG. 22E). In this orientation, the Ile+1 makes hydrophobic contacts with both benzene rings of 13 and 23, providing a rationale for the correlation of the size of the hydrophobic residue and the observed complex stabilization. With increasing size of the +1 amino acid, the residue fills more of the physical space between the two ring systems. The additional bromine of 23 pushes the 2-phenyl ring away from the roof of 14-3-3σΔC, explaining the lower activity towards Abl1, p65 and CFTR. Whilst a crystal structure of 28 with p65 was not obtained, given the structural similarities of the fragments, it can be assumed that 28 adopts a similar binding pose. The conformational change of 13 and 23 within the p65/14-3-3 complex illustrates how the functionality and topology of the binding partner influences ligand binding. Whilst direct hydrophobic contacts were observed with the fragments 13 and 23, compared with the Pin1_72/14-3-3 complex, there are significantly less interactions occurring at the composite interface within the p65/14-3-3 complex.

Finally, we performed a cooperativity analysis of the 14-3-3/p65/28 complex to investigate how these structural observations translate to cooperativity. For the 14-3-3/p65/28 complex, saturation of the system was not achieved at concentration ≤1 mM with SF_{1 mM}=37. This stabilization effect remains relatively small compared to the 270-fold stabilization of the Pin1/14-3-3 complex by 28 already achieved at lower concentrations. This lower cooperativity profile suggests that hydrophobic contacts of the phenyl and benzaldehyde rings of 28 with Ile+1 of p65 do not contribute significantly to stabilization of the ternary complex. More importantly, the lack of induced additional 14-3-3/p65 contacts upon binding of 28, as seen with Pin1, likely accounts for the disparity in cooperativity. The cooperative interactions within the 14-3-3/Pin1/28 complex are thus significant driving factors for the selective stabilization.

Targeting hub proteins, such as 14-3-3, via PPI modulation, raises the challenge of non-specific off target effects. Here we demonstrate a covalent imine-based tethering approach for de novo development of highly selective stabilizer fragments for the hub protein 14-3-3, within only a few focused library iterations. Critical to the development of selective stabilizers is location of the covalent anchor at the interface of the composite pocket. In contrast to anchor points peripheral to the interface, this approach biases fragments which are selective for a specific PPI interaction, by exploiting templating effects of the partner protein. We show that by harnessing unique topologies and functionalities within a composite binding pocket, unique fragments specific for the complex can be identified. Building upon these fragments to engage with the partner protein enabled the rapid identification of fragment based molecular glues which elicit sub-micromolar stabilizing activity. Further, we show how the 14-3-3/Pin1 complex can selectively be stabilized over other 14-3-3/complexes and demonstrate that the use of aldehydes as reversible covalent chemical probes does not lead to the inhibition of other 14-3-3 complexes formation. This highlights the advantage of using dynamic covalent tethering over non-reversible covalent bonds. Utilizing cooperative analysis and X-ray crystallography we elucidate the cooperativity of this series of fragments and the mechanism of action. Selectivity screening using a panel of 14-3-3 partner peptides identifies fragment 28 as highly selective for the Pin1 interaction. This is an important step forward in PPI stabilization of specifically hub proteins, such as 14-3-3, showing that a specific interaction can be stabilized over other interactions with a common binding motif. Finally, we show that by exploiting cooperative behavior we can drive selective complex formation. Specifically, we observed that direct communication through ligand-peptide interactions is critical to cooperativity, inducing additional interactions between the two protein partners that are relevant for the 14-3-3/Pin1/28 complex stability. The research shown here is relevant to the ongoing growth of molecular glues as drug targets.

### Example 10: Additional Compound Characterization

Protein Expression and Purification. The 14-3-3 proteins were recombinantly expressed in BL21(DE3) cells using pPROEX HTb vectors encoding for the 14-3-3σΔC (ΔC17 truncated C-terminus) and 14-3-3γ isoforms and TB medium. At a culture density of OD₆₀₀=0.8-1, protein expression was initiated with 0.4 mM IPTG for 18 h at 18° C. The cells were isolated by centrifugation (10.000×g, 15 min) and resuspended in lysis buffer (50 mM Tris/HCl pH8, 300 mM NaCl, 12.5 mM imidazole, 2 mM β-mercaptoethanol). A homogenizer was utilized for cell lysis, followed by centrifugation (40.000×g, 30 min) to clear the lysate. The proteins were purified using standard protocols for Ni-NTA-columns. The proteins were eluted with 250 mM imidazole (50 mM Tris/HCl pH8, 300 mM NaCl, 250 mM imidazole, 2 mM β-mercaptoethanol) and the full length 14-3-3γ was dialysis against 25 mM HEPES pH 7.5, 100 mM NaCl, 10 mM MgCl₂, 0.5 mM Tris(2-carboxyethyl)phosphine) and stored at −80° C. The 14-3-3σΔC for crystallography required removal of the His6-tag by TEV protease; the TEV was removed with Ni-NTA-columns. To ensure highest purity, the 14-3-3σΔC was applied to a size exclusion chromatography (20 mM HEPES pH7.5, 150 mM NaCl, 2 mM β-mercaptoethanol) and stored at −80° C.

Fluorescence Anisotropy (FA) Assays. Dissociation constants of binary complex formation were measured with a 1:1 dilution series of 14-3-3γ in the presence of 50 nM fluorescently labeled peptide. Stabilization factors (SF) were measured by a 1:1 dilution series of 14-3-3γ in the presence of 50 nM fluorescently labeled peptide and 100 μM compound or DMSO as control, with SF=K_{D,DMSO}/K_{D,compound}. For compound titrations, constant 50 μM of 14-3-3γ and 50 nM of fluorescently labeled Pin1_72 peptide was used, whereby the compound was titrated in a 1:1 dilution series. All FA assays were measured in FA buffer (10 mM HEPES pH 7.4, 150 mM NaCl, 0.1% Tween20, 1% BSA) in Corning 384 well plates (black, round bottom, low binding). Plates were incubated overnight prior to anisotropy measurements with the Tecan Infinite 500 plate reader (λ_{excitation}=485 nm, λₑₘᵢₛₛᵢₒₙ=535 nm).

TABLE 7
Overview of utilized peptide epitopes.
Given are the names as mentioned
in the main text, the binding site, the
N-terminal modifications with either
a fluorophore-linker construct or an
acetylation (ace.) for
crystallography, and the binding sequence.

| | | N-term. | |
|---|---|---|---|
| | Binding | modifi- | |
| Name | Site | cation | Binding Epitope |
| Pin1_71 | pS71 | FITC-Ahx | LVKHSQSRRP pS |
| | | | SWRQEK |
| | | | (SEQ ID NO: 43) |
| Pin1_72 | pS72 | FITC-Ahx/ | LVKHSQSRRPS pS |
| | | ace. | WRQEK |
| | | | (SEQ ID NO: 44) |
| p65 | pS45pS281 | FITC- | EGRSAG pS |
| | | βAla | IPGRRSGSG |
| | | | GGSGPSDREL pS |
| | | | EPMEFQ |
| | | | (SEQ ID NO:45) |
| p65_45 | pS45 | ace. | EGRSAG pS |
| | | | IPGRRS |
| | | | (SEQ ID NO: 46) |
| B-Raf | pS729 | FITC-Ahx/ | IHRSA pS |
| | | ace. | EPSLN |
| | | | (SEQ ID NO: 47) |
| C-Raf | pT259 | FITC-βAla | SQRQRST pS |
| | | | TPNVH |
| | | | (SEQ ID NO: 48) |
| AS160 | PT | FITC-Ahx/ | RRRAH pT FSHPP |
| | | ace. | (SEQ ID NO: 49) |
| Ab11 | pT735 | FITC-Ahx/ | EWRSV pT LPRDL |
| | | ace. | (SEQ ID NO: 50) |
| CFTR | pS753pS568 | HTC-βAla | AILPRI pS |
| | | | VISTGPTLQ |
| | | | ARRRQ pS |
| | | | VLNLMT |
| | | | (SEQ ID NO: 51) |
| Raptor | pS | FITC-Ahx | MRRAS pS |
| | | | YSSLN |
| | | | (SEQ ID NO: 52) |
| ERα | pT594 | FITC- | AEGFPA pT V-COOH |
| | | O1Pen | (SEQ ID NO: 14) |
| Mypt1 | pS472 | FITC-βAla | GVTRSA pS |
| | | | SPRLSS |
| | | | (SEQ ID NO: 53) |
| ERRγ | pS179 | FITC-Ahx | KRRRK pS |
| | | | CQA |
| | | | (SEQ ID NO: 54) |

X-Ray Crystallography. All binary crystals prepared by mixing 12 mg/ml 14-3-3σΔC in a 1:2 ratio with acetylated peptide in 20 mM HEPES pH7.5, 2 mM MgCl₂, 2 mM β-mercaptoethanol, followed by overnight incubation. Pin1_72/14-3-3σΔC crystals were grown in a hanging drop set up, whereby the complexation solution was mixed in 1:1 ratio with precipitation buffer (95 mM HEPES pH7.1, 27-28% PEG400, 190 mM CaCl₂), 5% glycerol). B-Raf/14-3-3σΔC and Abl1/14-3-3σΔC crystals were grown in a sitting drop set up. The complexation solution was mixed in 1:1 ratio with precipitation buffer (95 mM HEPES pH7.5, 27-28% PEG400, 190 mM CaCl₂, 5% glycerol). For AS160/14-3-3σΔC crystals the complexation solution was mixed in a 1:1 ration with the Wizard Cryo™ crystallization screen (Rigaku, Bainbridge Island, US), resulting in crystal growth with 40% (v/v) MPD and 100 mM CHES/Sodium hydroxide pH 9.5. All crystals were directly flash-frozen in liquid nitrogen and data acquisition took place at either the P11 beamline of PetraIII (DESY campus, Hamburg, Germany) or i-03/i-24 beamline of the diamond light source (Oxford, UK) or in-house.

Fragment screening was performed by crystal soaking, whereby a final concentration of 10 mM fragment was added to fully grown crystals (final DMSO ≤1%). The fragment/crystal mixtures incubated for seven days prior to data acquisition. The diffraction data were analyzed with the xia2/DIALS pipeline and MolRep was used for phasing. For model refinement Coot, Refmac5 and phenix.refine were utilized in iterative cycles. The elbow software of the phenix suite was used for ligand preparation based on fragment SMILES. Figures were generated with PyMOL© (V2.0.6, Schrodinger LLC).

Excitation/Emission Scans. Excitation/Emission profiles of fragments were measured at a final concentration of 1 mM in PBS buffer (137 mM NaCl, 2.7 mM KCl, 10 mM Na₂HPO₄, 1.8 mM KH₂PO₄). Measurements were performed with a Tecan Safire 2 plate reader (λ_{excitation}=230-730 nm; λₑₘᵢₛₛᵢₒₙ=280-830 nm; step size: 25 nm; gain: 60; lagtime: 0 μs) in a Corning 384-well plate (black, round bottom, low volume, low binding).

General materials. All reactions were prepared using AR or HPLC grade solvents without further purification. All reagents were purchase from Fluorochem, ABCR, Ak Scientific or Sigma-Adrich and were used without further purification unless stated. Microwave reactions were performed using a Biotage Initiator Plus equipped with a handling robot. Solvents were removed in vacuo using a Büchi rotary evaporator and a diaphragm pump. DMF and CH₂Cl₂ were dried and purified by means of a MBRAUN Solvent Purification System (MB-SPS-800). All other solvents used were of chromatography or analytical grade and supplied by Biosolve or Sigma-Aldrich. TLC was carried out on aluminum-backed silica (Merck silica gel 60 F254) plates supplied by Merck. Visualization of the plates was achieved using an ultraviolet lamp (λₘₐₓ=254 nm), 2,4-DNP, KMnO₄, anisaldehyde, bromine or ninhydrin. Column chromatography was either performed manually using silica gel (60-63 um particle size), automated Grace Reveleris X2 or Biotage Isolera chromatograph with prepacked silica columns supplied by Büchi/Grace (40 μm particle size). LC-MS analysis was carried out with a system comprising a Shimadzu Ion Trap Mass Spectrometer and C18 Jupiter SuC4300A 150×2.0 mm column using a gradient of 5-100% MeCN in water (+0.1% HCOOH) over 15 min. The purity of the samples was assessed using a UV detector at 254 nm. Unless otherwise stated all final compounds were >95% pure as judged by HPLC. GCMS analysis was performed on a Phenomenex Zebron ZB-5MS 30 m×0.25 mm×0.25 mm column with a gradient of 80° C. for 1 min to 300° C. for 1 min with a rate of 30° C./min in helium gas connected to a GCMS-QP2010 Plus Quadrupole Mass Spectrometer. High resolution mass spectra (HRMS) were recorded using a Waters ACQUITY UPLC I-Class LC system coupled to a Xevo G2 Quadrupole Time of Flight (Q-tof) mass spectrometer. Proton (¹H) and carbon (¹³C) NMR spectral data were collected on a 400 MHz Bruker Cryomagnet or 400 MHz Varian Gemini. Chemical shifts (6) are quoted in parts per million (ppm) and referenced to the residual solvent peak. Coupling constants (J) are quoted in Hertz (Hz) and splitting patterns reported in an abbreviated manner: app. (apparent), s (singlet), d (doublet), t (triplet), q (quartet), and m (multiplet). Assignments were made with the aid of 2D COSY, HMQC, and HMBC experiments.

General Procedure 1. To a microwave reaction tube was added 4-fluorobenzaldehyde derivative (1 eq), imidazole derivative (1.1 eq) and potassium carbonate (1.5 eq) in 2 mL of DMF. The reaction mixture was subject to microwave irradiation at 120° C. for 15 min. To the resulting reaction mixture was added water (10 mL) and the reaction mixture was subject to 2 min of ultra-sonication. The resulting precipitate was then filtered under vacuum, washed with water (2×3 mL) and dried under vacuum to afford the titled compound.

General Procedure 2. To a microwave reaction tube was added 4-fluorobenzaldehyde derivative (1 eq), imidazole derivative (1.1 eq) and potassium carbonate (1.5 eq) in 2 mL of DMF. The reaction mixture was subject to microwave irradiation at 120° C. for 15 min. To the resulting reaction mixture was added water (50 mL) and was extracted with ethyl acetate (2×50 mL). The organic layers were combined, washed with water (3×100 mL) and brine (100 mL). The organic layer was then separated, dried over sodium sulphate and concentrated under vacuum. The resulting crude residue was then subject silica column chromatography (gradient; hexane/EtOAc) to afford the titled compound.

3-bromo-4-(1H-imidazol-1-yl)benzaldehyde (1). Fragment 1 was synthesized according to general synthesis procedure 2 using 3-bromo-4-fluorobenzaldehyde (203 mg, 1.00 mmol), K₂CO₃ (207 mg, 1.50 mmol) and imidazole (75 mg, 1.10 mmol) to afford an amorphous cream solid (120 mg, 48%); ¹H NMR (400 MHz, DMSO-d6) δ 10.1 (s, 1H), 8.4 (d, J=1.7 Hz, 1H), 8.0 (dd, J=8.1, 1.8 Hz, 1H), 8.0 (s, 1H), 7.7 (d, J=8.0 Hz, 1H), 7.5 (s, OH), 7.1 (s, 1H); ¹³C NMR (101 MHz, DMSO) δ 191.8, 141.3, 138.1, 137.5, 135.2, 129.7, 129.6, 129.5, 121.3, 120.0.

2-chloro-4-(1H-imidazol-1-yl)benzaldehyde (2) Fragment 2 was synthesized according to general synthesis procedure 1 using 2-chloro-4-fluorobenzaldehyde (159 mg, 1.00 mmol), K₂CO₃ (207 mg, 1.50 mmol) and imidazole (75 mg, 1.10 mmol) to afford an amorphous cream solid (38 mg, 18%); ¹H NMR (400 MHz, Acetone-d6) δ 10.42 (s, 1H), 8.34 (s, 1H), 8.03 (d, J=8.5 Hz, 1H), 7.94 (d, J=2.1 Hz, 1H), 7.89-7.76 (m, 2H), 7.17 (s, 1H); ¹³C NMR (100 MHz, Acetone) δ 188.7, 143.2, 139.4, 136.6, 132.0, 131.9, 131.4, 122.5, 119.9, 118.4.

2-bromo-4-(1H-imidazol-1-yl)benzaldehyde (3). Fragment 3 was synthesized according to general synthesis procedure 2 using 2-bromo-4-fluorobenzaldehyde (100 mg, 0.49 mmol), K₂CO₃ (74.9 mg, 0.54 mmol) and imidazole (36.9 mg, 0.54 mmol) to afford an amorphous cream solid (91 mg, 74%); ¹H NMR (400 MHz, Acetone) δ 8.34 (s, 1H), 8.11 (d, J=2.2 Hz, 1H), 8.01 (d, J=8.5 Hz, 1H), 7.88 (dd, J=8.5, 2.1 Hz, 1H), 7.81 (s, 1H), 7.17 (s, 1H), 2.05 (p, J=2.2 Hz, 3H); ¹³C NMR (101 MHz, Acetone) δ 190.7, 143.2, 136.6, 132.5, 132.2, 132.0, 128.2, 125.7, 120.5, 118.4.

4-(4-methyl-1H-imidazol-1-yl)benzaldehyde (4). Fragment 4 was synthesized according to general synthesis procedure 1 using 4-fluorobenzaldehyde (200 mg, 1.61 mmol), K₂CO₃ (245 mg, 1.77 mmol) and 4-methylimidazole (146 mg, 1.77 mmol). The reaction mixture was diluted with water (50 mL) and was extracted with ethylacetate (2×50 mL). The resulting mixture was washed with sodium chloride solution (100 mL). The material was absorbed to silica and subject to automated column chromatography (0-100% Hexane:EtOAc) to afford the titled compound as cream solid (70 mg, 21.3%); ¹H NMR (400 MHz, Acetone) δ 10.05 (s, 1H), 8.15 (s, 1H), 8.05 (d, J=8.7 Hz, 2H), 7.82 (d, J=8.6 Hz, 2H), 7.44 (s, 1H), 2.21 (s, 3H). ¹³C NMR (101 MHz, Acetone) δ 191.7, 142.7, 140.9, 135.6, 135.5, 132.2 (2C), 120.9 (2C), 114.6, 13.9. NB: structural isomer [4-(3-methyl-1H-imidazol-1-yl)benzaldehyde] observed as a 7.4% impurity (based on proton NMR).

4-(4-(trifluoromethyl)-1H-imidazol-1-yl)benzaldehyde (6). Fragment 6 was synthesized according to general synthesis procedure 1 using 4-fluorobenzaldehyde (200 mg, 1.61 mmol), K₂CO₃ (245 mg, 1.17 mmol) and 4-(trifluoromethyl)-1Himidazol (241 mg, 1.17 mmol) to afford an amorphous cream solid (109 mg, 39%); ¹H NMR (400 MHz, Acetone-d6) δ 10.11 (s, 1H), 8.42 (s, 1H), 8.31 (s, 1H), 8.13 (d, J=8.6 Hz, 2H), 7.98 (d, J=8.6 Hz, 2H); ¹³C NMR (101 MHz, Acetone) δ 191.8, 141.6, 138.1, 136.8, 134.1, (q, J=38.5 Hz), 132.1 (2C), 122.8, (q, J=266.3), 122.5 (2C), 119.5 (q, J=4.0 Hz).

4-(1H-benzo[d]imidazol-1-yl)benzaldehyde (7). Fragment 7 was synthesized according to general synthesis procedure 2 using using 4-fluorobenzaldehyde (100 mg, 0.81 mmol), K₂CO₃ (123 mg, 0.89 mmol) and benzimidazole (105 mg, 0.89 mmol) to afford the titled compound as a brown solid (45 mg, 25%); ¹H NMR (400 MHz, Chloroform-d) δ 10.11 (s, 1H), 8.19 (s, 1H), 8.12 (d, J=8.5 Hz, 2H), 7.90 (dt, J=7.1, 3.6 Hz, 1H), 7.74 (d, J=8.4 Hz, 2H), 7.62 (dt, J=6.7, 3.5 Hz, 1H), 7.43-7.34 (m, 2H); ¹³C NMR (100 MHz, CDCl3) δ 190.8, 144.5, 141.9, 141.5, 135.4, 133.1 (2C), 131.8, 124.4, 123.9 (2C), 123.6, 121.2, 110.6.

1-(4-formylphenyl)-1H-imidazole-2-carboxylic acid (8). Fragment 2 was synthesized according to general synthesis procedure 2 using 2-chloro-4-fluorobenzaldehyde (124 mg, 1.00 mmol), K₂CO₃ (207 mg, 1.50 mmol) and imidazole (123 mg, 1.10 mmol) to afford the titled compound as a off-white amorphous solid (18 mg, 8%)¹H NMR (400 MHz, Acetone-d6) δ 10.08 (s, 1H), 8.27 (s, 1H), 8.09 (d, J=8.4 Hz, 2H), 7.89 (d, J=8.4 Hz, 2H), 7.17 (s, 1H); ¹³C NMR (100 MHz, Acetone) δ 192.7, 143.6, 137.4, 136.8, 133.1 (2C), 132.7, 122.4 (2C), 119.3.

4-(2-phenyl-1H-imidazol-1-yl)benzaldehyde (9). Fragment 9 was synthesized according to general synthesis procedure 2 using 4-fluorobenzaldehyde (100 mg, 0.81 mmol), K₂CO₃ (122.5 mg, 0.89 mmol) and 2-phenylimidazole (127.8 mg, 0.89 mmol) to afford the titled compound as an amorphous yellow oil (0.8 mg, 0.4%); ¹H NMR (400 MHz, DMSO-d6) δ 10.04 (s, 1H), 7.98 (d, J=8.4 Hz, 2H), 7.61 (d, J=1.3 Hz, 1H), 7.58-7.46 (m, 2H), 7.46-7.27 (m, 5H), 7.24 (d, J=1.3 Hz, 1H). ¹³C NMR (101 MHz, DMSO) δ 192.7, 146.3, 143.2, 135.7, 131.2 (2C), 130.6, 129.7, 129.0, 128.90 (2C), 128.85 (2C), 126.9 (2C), 123.9.

2-chloro-4-(2-phenyl-1H-imidazol-1-yl)benzaldehyde (10). Fragment 10 was synthesized according to general synthesis procedure 2 using 3-chloro-4-fluorobenzaldehyde (100 mg, 0.81 mmol), K₂CO₃ (116 mg, 0.84 mmol) and 2-phenylimidazole (90.1 mg, 0.63 mmol) to afford the titled compound as a brown solid (45 mg, 20%); ¹H NMR (400 MHz, Acetone) δ 10.41 (s, 1H), 7.92 (d, J=8.3 Hz, 1H), 7.57 (d, J=2.0 Hz, 1H), 7.51 (d, J=1.4 Hz, 1H), 7.42 (dd, J=7.5, 2.1 Hz, 2H), 7.39 (d, J=2.0 Hz, 1H), 7.38-7.29 (m, 3H), 7.21 (d, J=1.4 Hz, 1H). ¹³C NMR (101 MHz, Acetone) δ 189.0, 147.3, 144.8, 138.5, 132.5, 131.4, 131.2, 130.5, 129.7 (2C), 129.5, 129.2 (2C), 128.4, 126.0, 123.6.

3-chloro-4-(2-phenyl-1H-imidazol-1-yl)benzaldehyde (11). Fragment 11 was synthesized according to general synthesis procedure 2 using 3-chloro-4-fluorobenzaldehyde (159 mg, 1.00 mmol), K₂CO₃ (207 mg, 1.50 mmol) and 2-phenylimidazole (159 mg, 1.10 mmol) to afford an amorphous cream solid (31 mg, 11%); ¹H NMR (400 MHz, Acetone-d6) δ 10.12 (s, 1H), 8.12 (d, J=1.7 Hz, 1H), 8.04 (dd, J=8.0, 1.7 Hz, 1H), 7.77 (d, J=8.1 Hz, 1H), 7.39 (dd, J=7.4, 2.0 Hz, 2H), 7.35 (d, J=1.3 Hz, 1H), 7.31-7.21 (m, 4H); ¹³C NMR (100 MHz, Acetone) δ 191.2, 147.8, 142.1, 138.9, 133.4, 132.0, 131.61, 131.59, 130.3, 129.7, 129.3, 129.2 (2C), 128.5 (2C), 123.8.

3-bromo-4-(2-phenyl-1H-imidazol-1-yl)benzaldehyde (12). Fragment 12 was synthesized according to general synthesis procedure 2 using 3-bromo-4-fluorobenzaldehyde (100 mg, 0.49 mmol), K₂CO₃ (75 mg, 0.54 mmol) and 2-phenylimidazole (78 mg, 0.54 mmol) to afford the titled compound as an amorphous brown oil (9.7 mg, 6%); ¹H NMR (400 MHz, CDCl3) δ 10.44 (s, 1H), 9.35 (d, J=8.6 Hz, 1H), 8.00 (d, J=7.5 Hz, 1H), 7.76 (t, J=7.6 Hz, 1H), 7.70-7.57 (m, 1H), 7.49 (d, J=7.5 Hz, 1H), 7.30 (d, J=7.6 Hz, 2H), 7.23-7.01 (m, 3H). ¹³C NMR (101 MHz, CDCl3) δ 192.7, 135.7, 132.0, 131.7, 130.4, 130.1, 128.8, 128.7, 128.5 (2C), 127.9 (2C), 125.5, 124.7, 123.4.

2-bromo-4-(2-phenyl-1H-imidazol-1-yl)benzaldehyde (13). Fragment 13 was synthesized according to general synthesis procedure 1 using 2-bromo-4-fluorobenzaldehyde (100 mg, 0.49 mmol), K₂CO₃ (75 mg, 0.54 mmol) and 2-phenylimidazole (78.1 mg, 0.54 mmol to afford the titled compound as a brown solid (64 mg, 40%); ¹H NMR (400 MHz, Acetone-d6) δ 10.32 (s, 1H), 7.92 (d, J=8.3 Hz, 1H), 7.76 (d, 1H), 7.53 (s, 1H), 7.48-7.31 (m, 6H), 7.22 (s, 1H). ¹³C NMR (100 MHz, CDCl3) 0 190.5, 147.1, 143.9, 132.7, 130.9, 130.4, 130.3, 129.7, 129.3, 129.0 (2C), 128.7 (2C), 127.5, 125.2, 122.2.

4-(2-phenyl-1H-imidazol-1-yl)-2-(trifluoromethyl)benzaldehyde (14). Fragment 14 was synthesized according to general synthesis procedure 2 using 4-Fluoro-2-(trifluoromethyl)benzaldehyde (100 mg, 0.52 mmol), K₂CO₃ (79 mg, 0.57 mmol) and 2-phenylimidazole (82.6 mg, 0.57 mmol) to afford the titled compound as a brown solid (75 mg, 46%); ¹H NMR (400 MHz, Acetone) 0 10.36 (d, J=2.3 Hz, 1H), 8.19 (d, J=8.3 Hz, 1H), 7.88 (s, 1H), 7.78 (d, J=8.3 Hz, 1H), 7.63 (s, 1H), 7.51-7.31 (m, 6H), 7.27 (s, 1H); ¹³C NMR (100 MHz, Acetone-d6) 0 189.4 (q, J=2.1 Hz), 148.3, 144.6, 134.4 (d, J=1.5 Hz), 133.1, 132.7 (d, J=33.1 Hz), 132.2, 131.6, 130.7 (2C), 130.5, 130.1 (2C), 125.5 (q, J=6.0 Hz), 124.5.

4-(2-phenyl-1H-imidazol-1-yl)-3-(trifluoromethyl)benzaldehyde (15). Fragment 15 was synthesized according to general synthesis procedure 2 using 4-Fluoro-3-(trifluoromethyl)benzaldehyde (100 mg, 0.52 mmol), K₂CO₃ (96 mg, 0.69 mmol) and 2-phenylimidazole (75 mg, 0.52 mmol) to afford the titled compound as a brown solid (9 mg, 5.4%). ¹H NMR (400 MHz, DMSO-d6) 0 10.17 (s, 1H), 8.46 (d, J=1.8 Hz, 1H), 8.28 (dd, J=8.1, 1.8 Hz, 1H), 7.81 (d, J=8.1 Hz, 1H), 7.51 (brs, 1H), 7.29-7.24 (m, 6H); ¹³C NMR (101 MHz, DMSO-d6) 0 192.0, 147.2, 140.9 (d, J=1.7 Hz, *quartet not completely resolved), 137.1, 134.4, 132.9, 130.2, 129.2, 128.9 (2C), 127.9 (2C), 127.2 (q, J=31.2 Hz), 125.4, 122.9 (d, J=274.2 Hz, *quartet not completely resolved).

2-methoxy-4-(2-phenyl-1H-imidazol-1-yl)benzaldehyde (16). Fragment 16 was synthesized according to general synthesis procedure 2 using 2-methoxy-4-fluorobenzaldehyde (100 mg, 0.65 mmol), K₂CO₃ (89.7 mg, 0.65 mmol) and 2-phenylimidazole (62.4 mg, 0.43 mmol) to afford the titled compound as a brown solid (22.9 mg, 19%); ¹H NMR (400 MHz, CDCl3) 0 10.40 (s, 1H), 7.84 (d, J=8.3 Hz, 1H), 7.39 (dd, J=7.6, 2.0 Hz, 1H), 7.33-7.24 (m, 3H), 7.21 (s, 1H), 6.90 (dd, J=8.2, 1.2 Hz, 1H), 6.72 (d, J=1.9 Hz, 1H), 3.70 (s, 2H). ¹³C NMR (100 MHz, CDCl3) 0 207.1, 188.6, 162.2, 146.9, 144.6, 130.1, 123.0, 129.8, 128.9 (2C), 128.5 (2C), 124.0, 122.2, 117.6, 109.5, 56.0.

3-methoxy-4-(2-phenyl-1H-imidazol-1-yl)benzaldehyde (17). Fragment 17 was synthesized according to general synthesis procedure 1 using 3-methoxy-4-fluorobenzaldehyde (100 mg, 0.65 mmol), K₂CO₃ (99 mg, 0.71 mmol) and 2-phenylimidazole (103 mg, 0.71 mmol) to afford the titled compound as a brown solid (7 mg, 10%); ¹H NMR (400 MHz, Acetone) 0 10.07 (s, 1H), 7.65 (d, J=6.6 Hz, 2H), 7.54 (d, J=8.2 Hz, 1H), 7.43-7.36 (m, 2H), 7.33-7.22 (m, 4H), 7.20 (s, 1H), 3.70 (s, 3H). ¹³C NMR (100 MHz, Acetone) 0 192.1, 155.7, 147.9, 138.9, 133.6, 132.1, 129.9, 129.5, 129.1, 128.9 (2C), 128.4 (2C), 124.1, 124.0, 112.8, 56.4.

3-methyl-4-(2-phenyl-1H-imidazol-1-yl)benzaldehyde (18). Fragment 18 was synthesized according to general synthesis procedure 1 using 4-Fluoro-3-methylbenzaldehyde (100 mg, 0.72 mmol), K₂CO₃ (133.4 mg, 0.97 mmol) and 2-phenylimidazole (72.4 mg, 0.72 mmol) to afford the titled compound as an amorphous brown oil (12 mg, 6.6%); ¹H NMR (400 MHz, Acetone) 0 10.09 (s, 1H), 7.91 (d, J=7.1 Hz, 2H), 7.56 (d, J=8.7 Hz, 1H), 7.39 (dd, J=7.8, 1.9 Hz, 2H), 7.32-7.20 (m, 4H), 2.03 (s, 3H). ¹³C NMR (101 MHz, Acetone) δ 192.3, 143.9, 137.8, 137.2, 133.1, 131.8, 130.3, 129.6, 129.2, 129.1 (2C), 129.0, 128.4 (2C), 123.6, 17.5.

2-hydroxy-4-(2-phenyl-1H-imidazol-1-yl)benzaldehyde (19). Boron tribromide (1 M in DCM, 5 mL) was added dropwise to a solution of 2-methoxy-4-(2-phenyl-1H-imidazol-1-yl)benzaldehyde (16, 30 mg, 0.11 mmol) in anhydrous DCM (3 mL). The reaction was stirred at rt overnight after addition under an argon atmosphere. The reaction was quenched using ice water, followed by additional 5 M hydrochloride solution until the pH reached 1. The product was extracted using ethylacetate (2×50 mL). The resulting organic layer was washed with saturated sodium chloride solution (100 mL). The crude residue was absorbed to silica and subject to column chromatography (0-20% EtOAc:methanol) to afford the titled compound as a yellow solid (5 mg, 17%); ¹H NMR (400 MHz, Acetone-d6) δ 10.09 (s, 1H), 7.92-7.75 (m, 1H), 7.56-7.40 (m, 3H), 7.39-7.26 (m, 3H), 7.20 (d, J=1.3 Hz, 1H), 7.05-6.81 (m, 2H). 13C NMR (101 MHz, Acetone) δ 198.0, 187.1, 136.5, 132.6, 131.3, 130.4, 130.3 (2C), 130.02, 130.01 (2C), 124.5, 122.4, 122.1, 119.2, 115.8.

4-(2-phenyl-1H-imidazol-1-yl)-3-(trifluoromethoxy)benzaldehyde (20). Fragment 20 was synthesized according to general synthesis procedure 1 using 4-Fluoro-3-(trifluoromethoxy)benzaldehyde (100 mg, 0.48 mmol), K₂CO₃ (66 mg, 0.48 mmol) and 2-phenylimidazole (46 mg, 0.32 mmol) to afford the titled compound as an amorphous brown oil (64 mg, 60%); ¹H NMR (400 MHz, Acetone-d6) δ 10.15 (s, 1H), 8.10 (d, J=8.1 Hz, 1H), 8.00 (s, 1H), 7.83 (d, J=8.1 Hz, 1H), 7.42 (s, 1H), 7.38 (d, J=5.7 Hz, 2H), 7.33-7.28 (m, 4H); ¹³C NMR (101 MHz, Acetone-d6) δ 191.1, 148.1, 144.8 (q, J=1.6 Hz), 138.7, 136.8, 131.4, 131.4, 130.4, 129.9, 129.4, 129.1 (2C), 128.6 (2C), 124.0, 122.4, 121.0 (q, J=259.2 Hz).

3-phenoxy-4-(2-phenyl-1H-imidazol-1-yl)benzaldehyde (21). Fragment 21 was synthesized according to general synthesis procedure 1 using 3-phenoxy-4-fluorobenzaldehyde (100 mg, 0.46 mmol), K₂CO₃ (64 mg, 0.46 mmol) and 2-phenylimidazole (44.5 mg, 0.31 mmol to afford the titled compound as an amorphous brown oil (17.5 mg, 17%); ¹H NMR (400 MHz, Acetone-d6) δ 10.00 (s, 1H), 7.79 (dd, J=21.9, 8.0 Hz, 2H), 7.45 (d, J=7.4 Hz, 2H), 7.39 (s, 1H), 7.36-7.27 (m, 6H), 7.21-7.14 (m, 2H), 6.66 (d, J=8.0 Hz, 2H); ¹³C NMR (101 MHz, Acetone) δ 191.7, 155.5, 154.1, 148.1, 138.6, 134.8, 132.3, 131.1 (2C), 130.5, 130.0, 129.2, 129.1 (2C), 128.6 (2C), 125.8, 125.7, 124.0, 120.6 (2C), 117.8.

4-(2-phenyl-1H-imidazol-1-yl)-1-naphthaldehyde (22). Fragment 22 was synthesized according to general synthesis procedure 1 using 4-fluoro-1-naphtaldehyde (100 mg, 0.57 mmol), K₂CO₃ (87 mg, 0.63 mmol) and 2-phenylimidazole (91 mg, 0.63 mmol to afford the titled compound as a yellow solid (44 mg, 26%); ¹H NMR (400 MHz, Acetone-d6) δ 10.52 (s, 1H), 9.35 (d, J=8.6 Hz, 1H), 8.29 (d, J=7.5 Hz, 1H), 7.84-7.77 (m, 2H), 7.66 (t, J=7.7 Hz, 1H), 7.52-7.44 (m, 2H), 7.38-7.31 (m, 3H), 7.23-7.09 (m, 3H); ¹³C NMR (100 MHz, Acetone) δ 193.9, 148.4, 141.7, 136.8, 133.0, 132.2, 131.4, 131.3, 130.6, 130.1, 129.3, 129.2, 129.0 (2C), 128.4 (2C), 126.0, 125.9, 125.3, 124.0.

2-bromo-4-(2-(2-bromophenyl)-1H-imidazol-1-yl)benzaldehyde (23). Fragment 23 was synthesized according to general synthesis procedure 2 using 3-bromo-4-fluorobenzaldehyde (120 mg, 0.6 mmol), K₂CO₃ (80 mg, 0.6 mmol) and 2-(2-Bromophenyl)-1H-imidazole (90 mg, 0.4 mmol) to afford an amorphous off white solid (94 mg, 58%); ¹H NMR (400 MHz, Acetone-d6) δ 10.25 (s, 1H), 7.84 (d, J=8.4 Hz, 1H), 7.67 (s, 1H), 7.65-7.59 (m, 5H), 7.52 (t, J=7.5 Hz, 2H), 7.43 (t, J=7.7 Hz, 2H), 7.37 (d, J=8.3 Hz, 2H), 7.26 (s, 1H); 13C NMR (100 MHz, Acetone) δ 190.8, 146.2, 144.0, 134.0, 133.70, 133.67, 133.1, 132.2, 131.3, 130.5, 130.3, 128.7, 126.9, 124.9, 124.5, 121.9.

2-bromo-4-(2-(4-bromophenyl)-1H-imidazol-1-yl)benzaldehyde (24). Fragment 24 was synthesized according to general synthesis procedure 2 using 3-bromo-4-fluorobenzaldehyde (300 mg, 1.5 mmol), K₂CO₃ (210 mg, 1.5 mmol) and 2-(4-Bromophenyl)-1H-imidazole (220 mg, 1.0 mmol) to afford an amorphous cream solid (19 mg, 5%); ¹H NMR (400 MHz, Acetone-d6) δ 10.33 (s, 1H), 7.95 (d, J=8.3 Hz, 1H), 7.82 (d, J=2.0 Hz, 1H), 7.54 (d, J=8.9 Hz, 3H), 7.47 (dd, J=8.4, 2.0 Hz, 1H), 7.38 (d, J=8.5 Hz, 2H), 7.23 (s, 1H); 13C NMR (100 MHz, Acetone) δ 190.9, 146.2, 144.5, 133.8, 132.4 (2C), 131.63, 131.60, 131.4 (2C), 130.7, 130.5, 127.3, 126.6, 124.1, 123.3.

2-bromo-4-(2-(4-hydroxyphenyl)-1H-imidazol-1-yl)benzaldehyde (25). Fragment 25 was synthesized according to general synthesis procedure 1 using 3-bromo-4-fluorobenzaldehyde (300 mg, 1.5 mmol), K₂CO₃ (210 mg, 1.5 mmol) and 4-(1H-Imidazol-2-yl)phenol (160 mg, 1.0 mmol) to afford an amorphous cream solid (66 mg, 19%); ¹H NMR (400 MHz, Acetone-d6) δ 11.71 (s, 1H), 10.23 (s, 1H), 8.11 (d, J=8.7 Hz, 2H), 7.92 (d, J=8.7 Hz, 1H), 7.31 (d, J=2.4 Hz, 1H), 7.30-7.21 (m, 4H), 7.16 (dd, J=8.7, 2.4 Hz, 1H), 7.09 (s, 1H). 13C NMR (100 MHz, Acetone) δ 191.3, 164.8, 156.2, 147.1, 133.4, 131.3, 130.55, 130.45, 129.3, 128.8 (2C), 123.6, 122.5 (2C), 119.0, 118.8.

2-bromo-4-(2-(pyridin-3-yl)-1H-imidazol-1-yl)benzaldehyde (26). Fragment 26 was synthesized according to general synthesis procedure 2 using 3-bromo-4-fluorobenzaldehyde (300 mg, 1.5 mmol), K₂CO₃ (210 mg, 1.5 mmol) and 3-(1H-Imidazol-2-yl)-pyridine (150 mg, 1.0 mmol) to afford an amorphous cream solid (120 mg, 37%); ¹H NMR (400 MHz, Acetone-d6) δ 10.33 (s, 1H), 8.63 (d, J=2.2 Hz, 1H), 8.54 (dd, J=4.9, 1.6 Hz, 1H), 7.95 (d, J=8.3 Hz, 1H), 7.85 (d, J=2.0 Hz, 1H), 7.75 (d, J=8.0 Hz, 1H), 7.60 (d, J=1.3 Hz, 1H), 7.51 (dd, J=8.3, 2.0 Hz, 1H), 7.34 (dd, J=8.0, 4.8 Hz, 1H), 7.28 (d, J=1.3 Hz, 1H). 13C NMR (100 MHz, Acetone) δ 190.0, 149.4, 149.3, 143.8, 143.4, 135.6, 133.0, 130.9, 130.8, 130.0, 126.5, 126.5, 125.8, 123.4, 123.1.

2-bromo-4-(2-(5-bromo-2-fluorophenyl)-1H-imidazol-1-yl)benzaldehyde (27). Fragment 27 was synthesized according to general synthesis procedure 1 using 3-bromo-4-fluorobenzaldehyde (203 mg, 1.0 mmol), K₂CO₃ (152 mg, 1.1 mmol) and 2-(2,4-difluorophenyl)-1H-imidazole (176 mg, 1.1 mmol) to afford an amorphous beige solid (80 mg, 19%); ¹H NMR (399 MHz, Acetone-d6) δ 10.30 (s, 1H), 7.91 (d, J=8.3 Hz, 1H), 7.87 (dd, J=6.4, 2.5 Hz, 1H), 7.79 (s, 1H), 7.68 (d, J=11.6 Hz, 2H), 7.47 (d, J=8.3 Hz, 1H), 7.31 (s, 1H), 7.18-6.96 (m, 1H). ¹³C NMR (100 MHz, Acetone-d6) δ 189.9, 159.7, 157.2, 143.3 (d, J=2.2 Hz), 140.4 (d, J=1.3 Hz), 134.7 (d, J=3.0 Hz), 134.4 (d, J=8.5 Hz), 132.7, 130.5 (d, J=29.6 Hz), 129.2, 126.3, 124.0, 122.7, 121.1 (d, J=16.2 Hz), 117.9 (d, J=23.6 Hz), 116.6 (d, J=3.4 Hz).

2-bromo-4-(2-(2,4-difluorophenyl)-1H-imidazol-1-yl)benzaldehyde (28). Fragment 28 was synthesized according to general synthesis procedure 1 using 3-bromo-4-fluorobenzaldehyde (203 mg, 1.0 mmol), K₂CO₃ (152 mg, 1.1 mmol) and 2-(2,4-difluorophenyl)-1H-imidazole (198 mg, 1.1 mmol) to afford an amorphous beige solid (62 mg, 17%); ¹H NMR (400 MHz, DMSO-d6) δ 10.17 (s, 1H), 7.84 (d, J=8.4 Hz, 1H), 7.78 (dd, J=16.9, 1.8 Hz, 2H), 7.68 (td, J=8.2, 6.3 Hz, 1H), 7.35 (ddd, J=8.3, 2.1, 0.7 Hz, 1H), 7.30 (d, J=1.4 Hz, 1H), 7.28-7.20 (m, 2H); ¹³C NMR (101 MHz, DMSO-d6) δ 190.7, 163.0 (dd, J=249.4, 12.3 Hz), 159.1 (dd, J=250.3, 12.8 Hz), 142.6 (d, J=1.4 Hz), 140.3 (d, J=1.0 Hz), 133.6 (dd, J=10.0, 4.0 Hz), 132.0, 131.0, 129.8, 129.0, 125.9, 123.9, 122.7, 115.1 (dd, J=14.9, 3.8 Hz), 112.4 (dd, J=21.7, 3.5 Hz), 104.5 (t, J=26.0 Hz).

### Example 11: Cooperative Protein-Protein Stabilizers—Utilizing a Reversible Covalent Tethering Approach

Targeting the p65 subunit of NF-κB is of specific interest since NF-κB is a homo- and/or heterodimeric transcription factor involved in the regulation of immune responses, cell proliferation and inflammation, therefore connected to cancer and autoimmune diseases amongst others. Attempts to directly inhibit the transcriptional activity of NF-κB have typically failed due to the inability to identify NF-κB-targeting matter. Interestingly, increased transcriptional activity of p65 has been correlated with downregulation of 14-3-3 in studies on ischemia-reperfusion and breast cancer. Also, upregulation of 14-3-3 has been shown to favor cytosolic localization of p65, subsequently preventing transcriptional activity. Stabilization of the 14-3-3/p65 complex could therefore furnish a novel entry point for targeting NF-κB and enabling a controlled therapeutic intervention.

Point mutational studies on p65 revealed three potential 14-3-3 binding sites, surrounding the phosphorylation sites S45, S281 and S340. Binding affinities and structural information for two of these sites, pS45 and pS281, were gained, showing the direct physical interaction between both proteins. Benzaldehyde-based molecular fragments were shown to bind specifically to Lys122 of 14-3-3 via imine bond formation, thereby stabilizing the interaction with the p65 motif around phosphorylation site pS45, via hydrophobic contacts with p65.

Here, we provide additional data on an imine-based site directed fragment approach to develop a 14-3-3/p65 molecular glue. Critical to the development of molecular glues is a robust understanding of molecular interactions and structural changes in the protein-protein-ligand complex that result in cooperative behavior. To understand the chemical properties that produce cooperative ligands we employed a fragment extension design process, using structural information gathered from X-ray crystallography soaking experiments and fluorescence anisotropy (FA) measurements. This enabled us to designed initial fragments into molecular glues which showed stabilizing activity for the 14-3-3/p65 complex; culminating with the discovery of compound 241 which elicits an 81-fold stabilizing effect on the 14-3-3/p65 complex.

We have previously shown that aldimine bond formation is highly selective for Lys122 of 14-3-3 which lies at the interface between 14-3-3 and p65 in the composite binding pocket. The enhanced selectivity for Lys122 is the result of a combination of the local hydrophobic character of the composite pocket, a lowered pKₐ of the lysine side chain, and the templating effects of p65 binding. Given the intrinsically disordered nature of large parts of the p65 subunit of NF-κB, we utilized a 13-mer phosphopeptide representing (EGRSAG pSer45 IPGRRS (SEQ ID NO:9)) the recognition sequence of 14-3-3 to expedite chemical matter elucidation. Our initial investigation used X-ray crystal soaking experiments and a fragment library of commercially available aldehydes (34 fragments) to identify four key chemotypes that induced imine bond formation with Lys122 within the 14-3-3/p65 composite pocket; methylsulfonyl (1 and 2), 1-nitro,3-hydroxybenzene (3), methyl acetamide (4) and five-membered N-heterocycles (5, 6 and 7). Biochemical assessment of fragments 1-7 used a fluorescence anisotropy (FA) assay. To assess the fragments capacity to induce a ternary complex the fragments were titrated to a solution of 100 nM fluorescently labeled p65_45 peptide (FITC-βAla-EGRSAG pSer45 IPGRRS (SEQ ID NO:9)) and 50 μM of 14-3-3γ. The subsequent outputs are herein term termed ‘half maximal complex concentration (CC₅₀)’. Results from the assay showed that these fragments did not increase complex formation. However, the lack of increase in complexation at relevant fragment concentrations was in accordance with crystal data, which indicated little to no direct contacts between the fragments and the p65 peptide. Fragments which are detectable in crystallography experiments but do not induce a detectable effect on complex formation are termed, ‘silent-binders’. We sought to develop a structure-activity relationship (SAR) based upon these four chemotypes with the specific goal to develop high affinity molecular glues which orthosterically engage with p65 and stabilize the 14-3-3/p65 complex.

To facilitate rapid optimization of the initial hit compounds into molecular glues, six focused libraries were synthesized based on the four chemotypes identified from the initial screen. Key to the development of molecular glues is understanding the interaction between the fragments and the 14-3-3/p65 complex which leads to cooperative binding. Within our lab we have developed a highly robust crystallography screening system to rapidly access X-ray crystal structures of soaked fragments. Utilizing this system, we gain unparalleled structural information which guides hit optimization.

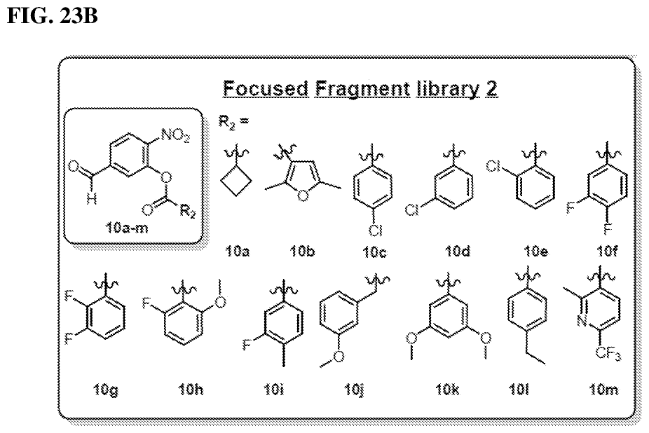

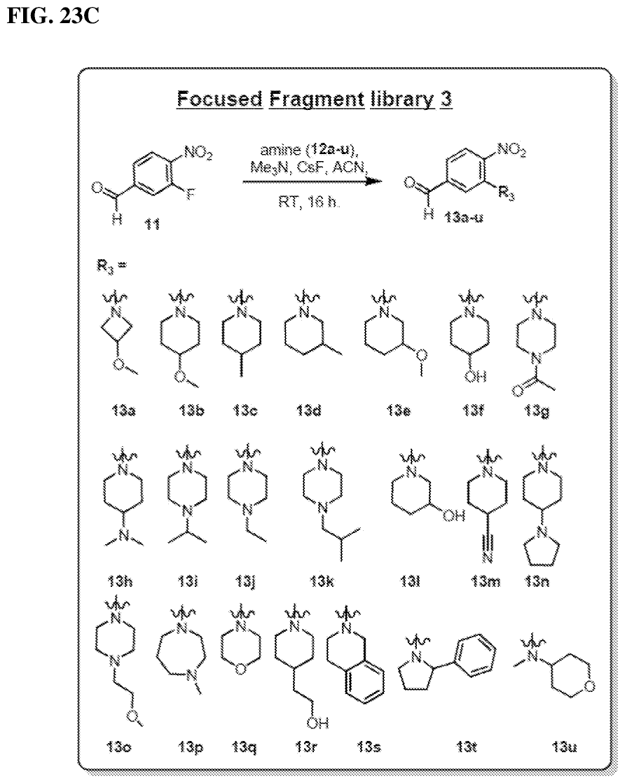

Focused library development around 3-hydroxy,4-nitrobenzaldehyde scaffold. Initial focused library development concentrated on extension of 3 based on crystal data, which showed complete coverage of the fragment by the electron density map, indicating a high occupancy for 3 within the composite pocket. Analysis of the X-ray crystal structure of 3 indicated that a relatively large solvent exposed pocket was present in front of the fragment formed by p65. We sought to explore this chemical space via extension of 3 from the 3-hydroxy position. Focused libraries 1 and 2 were synthesized using either a sulfonylation reaction with a sulfonyl chloride (8a-1) or an esterification using carboxylic acid chlorides (10a-m) (FIGS. 23A-23B).

Analysis of focused libraries 1 and 2 using X-ray crystal soaking experiments showed three sulfonate fragments (9a-c) and four ester fragments (11a, d, j and m) bound to Lys122 in the crystal structures. The various substituents elicited poor to moderate coverage by the electron density map, except for the sulfonetes 9a and 9b. However, neither 9a nor 9b were active in functional FA assays. For these smaller substitutes (9a and 9b), no favorable contacts with the peptide could be observed, whereas the larger substitutions resulted in a loss of electron density, indicative of a high conformational freedom of the ester side chain (9c, 11a, and 11j).

An additional collection of fragments (focused library 3) was synthesized based on tertiary amines at the meta-position. The replacement of the phenol oxygen of 3 with a ternary amine increased the number of vectors for fragment extension, reduced conformational freedom and enabled the exploration of different chemical space. A one-step nucleophilic aromatic substitution using key benzaldehyde intermediate 11 and the corresponding amine (12a-u) was employed to afford 21 analogues (13a-u) with yields ranging from 20-68% (FIG. 23C). Focused fragment library 3 was then soaked into p65_45/14-3-3 crystals and tested in FA assay. Of this collection, four fragments bound in crystallography experiments, with 13e, f, l and q showing significant electron density coverage. Notably, all four fragments contained six membered saturated rings with polar functional groups which formed either direct hydrogen bonds with the backbone of p65 or water mediated hydrogen bonds. The binding poses of these compounds were well defined, whereby the saturated 6-membered ring system extends towards the C-terminus of the p65 peptide. These results suggest that these fragments are shielded by the amphiphilic amino acids Pro47, Gly48, Arg49 and Arg50 of p65, which facilitates covalent tethering. Biophysical assessment of 13e, f, l and q using FA compound titration assays showed that the fragments did not elicit significant ternary complex formation at biochemically relevant concentrations. Surprisingly, 13f and 13l showed no increased complex formation, considering both fragments engage in hydrogen bonding with p65. Given the lack of 14-3-3/p65 increased complex formation in FA compound titration assays at concentrations practical for hit optimization we shifted focus to the five-membered N-heterocycles chemotype.

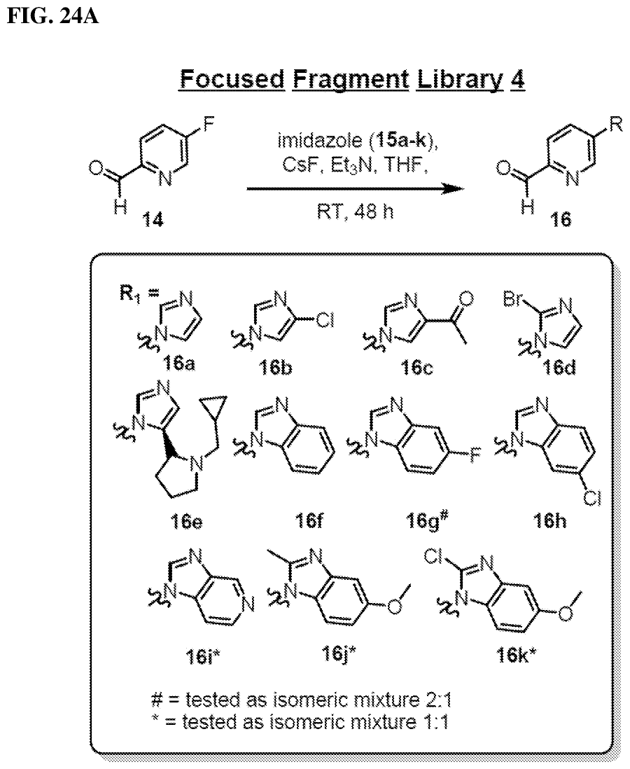

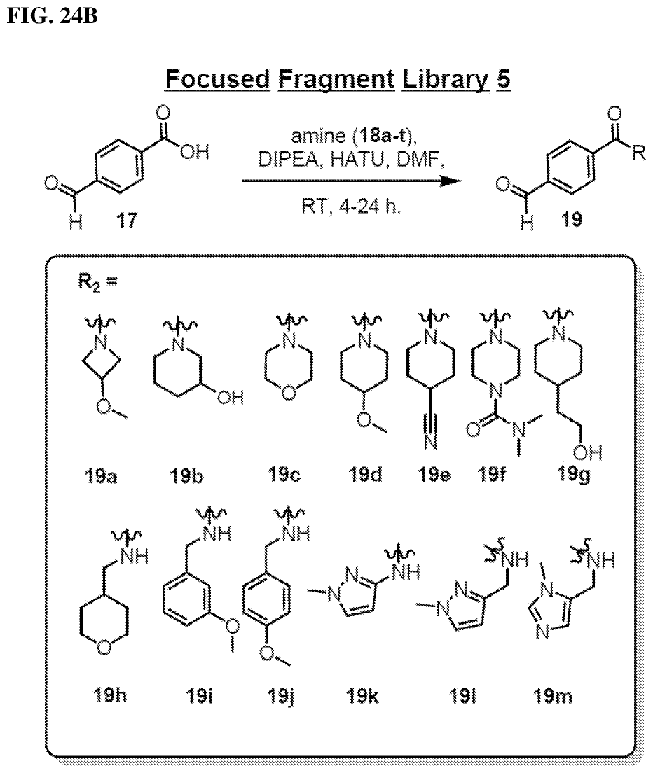

Focused library development around 4-(1H-imidazol-1-yl) benzaldehyde scaffold. Fragment 5 was selected for fragment library development as the para-substitution proved to be more solvent exposed compared with 7 and the 1,3-substituted imidazole provided the possibility for fragment extension from three vectors of the N-heterocycle. A focused library of 13 fragments (focused library 4) was synthesized using a nucleophilic aromatic substitution reaction with cesium fluoride, triethylamine, 4-fluoro-nicotinaldehyde (14), and an array of substituted imidazoles (15a-e) or benzimidazoles (15f-k) (FIG. 24A). The starting reactant 4-fluoro-nicotinaldehyde (14), was used to improve solubility of the fragments and to provide a further point for a polar interaction compared with 5-7. The resulting library was then subject to X-ray crystal soaking experiments and FA assay. Notably, fragments 16b, c, f and g-k were tested as mixtures of regioisomers.

Analysis of the fluorescence anisotropy assay identified that fragments 16d, e, j and k induced an increase in anisotropy. However, only 16d, j and k were detected in the electron density map of soaking experiments. Notably, all three fragments showed a mixture of conformational poses. Both regioisomers of 16j (2-methyl-5-methoxy-benzimidazole) and 16k (2-chloro-5-methoxy-benzimidazole) were observed to bind within the composite binding pocket, as result of soaking experiment using regioisomeric mixtures. For instance, the chlorine atom in 16k points towards the p65 peptide, while the methoxy-substitution can be detected in the 5-position. For the other binding pose, the chlorine atom in 16k points to the FC pocket, whereas the methoxy-substitution located in the 6-position is positioned above Ille46 of p65. Both regioisomers of 16k engage in hydrophobic contacts with the roof of 14-3-3 and 11146 of p65_45. Despite their similar binding poses, 16k (CC₅₀=260 μM) elicited a significantly higher increase in anisotropy compared to 16j which induced a negligible ternary complex formation in FA assay. The replacement of a chloro-moiety for a methyl group in 16k (16j) on the imidazole ring has a significant impact on complex formation, resulting in diminished anisotropy.

Focused library development around 4-formylbenzamide scaffold. We next turned our attention to fragment extension based upon fragment 4. Analysis of the co-crystal structure showed the N-acetyl group probes up and toward the p65 peptide. In order to expediate rapid parallel synthesis of focused library 5, N-(4-formylphenyl)acetamide (4) was inverted to a N-substituted 4-Formyl-benzamide (19a-t), improving the nucleophilicity of the amine in the corresponding amide coupling reactions, as well as providing greater chemical diversity of the fragment extension (FIG. 24B). Further, previously published cluster analysis of occurring torsion (t) angles of benzamide report a t≈300 and 150° between the benzene ring and the acetamide head group. We hypothesized that extension of the fragment from this vector provides the opportunity to engage with the p65 peptide and enhance fragment binding. Focused library 5 was synthesized using standard amide coupling conditions, 1-formylbenzoic acid (17) and amines 18a-t. In total, 13 fragments (19a-t) with amide substitution in the para-position of the aldehyde functionality were synthesized, soaked in p65_45/14-3-3σΔC crystals and tested in FA compound titrations. All fragments from library 5 were shown to bind within the composite binding pocket using X-ray crystallography. Subsequent biophysical analysis identified that all compounds were silent binders, not inducing detectable formation of a ternary complex at assay relevant concentrations. X-ray co-crystallization experiments and an assessment of the C—C—C—N torsion angles provided an explanation for the lack of activity for this series of fragments. The electron density map of the aldehydes showed highly resolved electron density for the benzaldehyde ring and the amide of the benzamide. The carbonyl of the benzamide engages in polar contacts with the water shell of 14-3-3, thereby stabilizing this orientation of the fragments. The binding poses of the different R-substitutions are poorly resolved by the electron density map suggesting a high level of conformational freedom and a high-level entropy of the substitutes, unfavorable for binding. Assessment of this library of fragments showed the R-substituted pointing towards the solution above the p65 peptide or Asp215 of 14-3-3. For a few fragments the R-substitutes engage in polar contacts with the 14-3-3 water shell, exemplified by 19 h. However, the additional polar contacts do not translate to significant increase in ternary complex formation. Given the lack of ternary complex formation of this library at biochemically relevant concentration we shifted focus to the 4-formyl benzenesulfonamide chemotype.

Focused library development around 4-formyl-benzenesulfonamides. Having investigated focused libraries 1-5, we shifted our attention to development of a focused library based on fragment 1. Analysis of the crystal structure of fragment 1, specifically the torsion angle of i=900±30° between the benzene ring and the mesyl group proved interesting. Extension of the fragment from the methyl provided a potential point of reaching over the p65 peptide trapping its binding to 14-3-3. Alternatively, we postulated that fragment extension could also result in a change in the conformation of the fragment leading to the occupation of the FC-pocket and increasing 14-3-3 based affinity. To expedite fragment development the methyl group was replaced for N-substituted amines to facilitate rapid access to a library of 25 structural analogues of 4-formyl-benzenosulfonamides. This library of sulfonamides was synthesized by conversion of sodium 2-formyl-benzene-1-sulfonate (20) to 4-formyl-benzenesulfonyl chloride (21) using thionyl chloride in DMF. Subsequent coupling of 21 with N-substituted amines (22a-z) afforded fragments 23a-z.

Table 8. Exploration of 4-formyl-benzenesulfonamides. CC₅₀: values of compound titrations with 50 μM 14-3-3γ. K_{D,app}: value of protein titrations in presence of 1 mM of fragment. SF: the fold-change of apparent K_{D} in comparison to a DMSO control. All fragments bound to the p65_45/14-3-3σΔC complex.

Focused Fragment Library 6

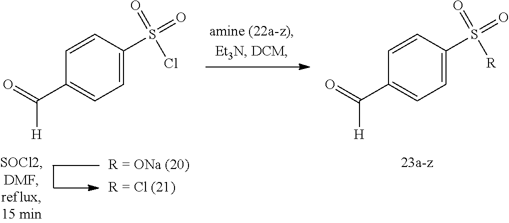

| | | CC₅₀ | K_{D},app | |
|---|---|---|---|---|
| No. | R | (μM) | (μM) | SF |
| 23a | 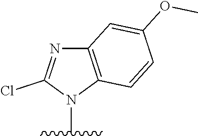 [SMILES: *n1c(Cl)nc2cc(OC)ccc21] | >1000 | ND | ND |
| 23b | 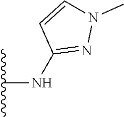 [SMILES: *Nc1ccn(C)n1] | >1000 | 220 | 1.5 |
| 23c | 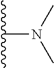 [SMILES: *N(C)C] | >1000 | 180 | 1.8 |
| 23d | 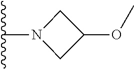 [SMILES: *N1CC(OC)C1] | 940 | 65 | 5.1 |
| 23e | 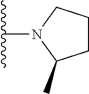 [SMILES: *N1CCC[C@H]1C] | 280^{a} | 16 | 20.6 |
| 23f | 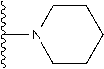 [SMILES: *N1CCCCC1] | 340^{a} | 15 | 22.0 |
| 23g | 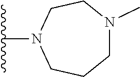 [SMILES: *N1CCCN(C)CC1] | 640^{a} | 82 | 4.0 |
| 23h | 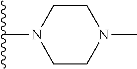 [SMILES: *N1CCN(C)CC1] | >1000 | 100 | |
| 23i | 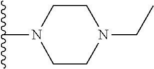 [SMILES: *N1CCN(CC)CC1] | 790^{a} | 60 | 3.3 |
| 23j | 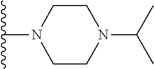 [SMILES: *N1CCN(C(C)C)CC1] | >1000 | 180 | 5.5 |
| 23k | 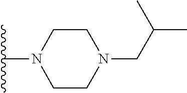 [SMILES: *N1CCN(CC(C)C)CC1] | 510^{a} | 27 | 1.8^{a} |
| 23l | 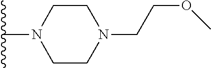 [SMILES: *N1CCN(CCOC)CC1] | 550 | 74 | 4.5 |
| 23m | 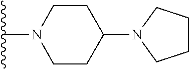 [SMILES: *N1CCC(N2CCCC2)CC1] | 550 | 94 | 3.5 |
| 23n | 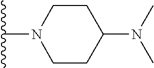 [SMILES: *N1CCC(N(C)C)CC1] | 930^{a} | 93 | 3.5 |
| 23o | 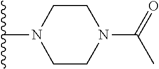 [SMILES: *N1CCN(C(C)=O)CC1] | >1000 | 74 | 4.5 |
| 23p | 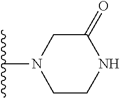 [SMILES: *N1CCNC(=O)C1] | >1000 | 150 | 2.2 |
| 23q | 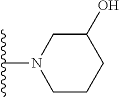 [SMILES: *N1CCCC(O)C1] | >1000^{a} | 32 | 10.3 |
| 23r | 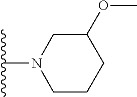 [SMILES: *N1CCCC(OC)C1] | >1000^{a} | 44 | 7.5 |
| 23s | 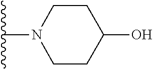 [SMILES: *N1CCC(O)CC1] | 540 | 46 | 7.2 |
| 23t | 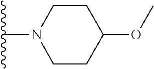 [SMILES: *N1CCC(OC)CC1] | 650 | 20 | 16.5 |
| 23u | 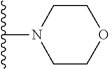 [SMILES: *N1CCOCC1] | 650 | 52 | 6.3 |
| 23v | 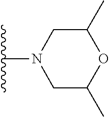 [SMILES: *N1CC(C)OC(C)C1] | 430 | 31 | 10.6 |
| 23w | 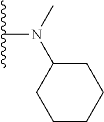 [SMILES: *N(C)C1CCCCC1] | 220 | 26 | 12.7 |
| 23x | 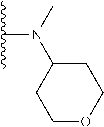 [SMILES: *N(C)C1CCOCC1] | 680 | 56 | 5.9 |
| 23y | 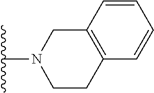 [SMILES: *N1CCc2ccccc2C1] | 180 | 450 | 0.7 |
| 23z | 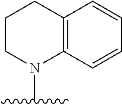 [SMILES: *N1CCCc2ccccc21] | 57 | 5.1 | 64.7 |

^{a} = fit did not converge; ^{b} = tested as isomeric mixture 1:1; ND = not determined

In contrast to focused libraries 1-5 most of the tested 4-formyl-benzenesulfonamide fragments showed significant stabilization in FA assays, allowing a differentiated SAR analysis (Table 8). The activity ranged from a weak affinity with a small, not quantifiable increase in anisotropy, to a two-digit micromolar CC₅₀ potency in compound titrations. From the focused library 6, nine fragments elicited CC₅₀ values ranging from 57-430 μM. An additional assessment of stabilization using FA protein titration assay showed for 19 fragments a significant shift of apparent K_{D} values in presence of 1 mM the fragments. Fragments 23e, f, k, t, w, and z showed K_{D} values of <30 μM compared to the binary complex of full-length 14-3-3 and the p65pSer45 peptide which gave a K_{D}=300±100 μM. Analysis of this subset of fragments showed that these shifts in K_{D}S (Stabilization Factors, SFs) range from ˜10-65-fold. Fragment 23z, possessing a N-substituted 1,2,3,4-tetrahydroquinoline, showed the greatest SF of ˜65-fold.

Comparison of X-ray crystal soaks provided an explanation for the FA assay results. Structural data showed that 2-methyl pyrrolidine (23e) and piperidine (23f) were tolerated within the composite binding pocket. Ring expansion to the N-methyl diazepane (23g) proved to be detrimental to stabilization (EC₅₀=640 μM). Notably, polar functionalities within six membered N-heterocycles were not well tolerated, specifically hydrogen bond accepting and donating groups (23h, n-u) elicited high CC₅₀ values and modest SFs (6-16-fold). This can be exemplified by fragment 23f containing piperidine (app K_{D}=15 μM, SF=22) compared with N-methylpiperazine 23h (app K_{D}=100 μM, SF=3.3) and morpholine 23u (app K_{D}=52 μM, SF=6.3). Analysis of the X-ray structures showed that introduction of a polar functionality typically resulted in reorientation of the R-substitute of the fragment towards the FC-binding pocket. Introduction of a hydrophobic functionality such as 23k (app K_{D}=27 μM, SF=12.2) and 23v (app K_{D}=430 μM, SF=10.6) recovered stabilizing activity, with the fragments re-establishing hydrophobic contacts with the p65 peptide. Notably, large amphiphilic modifications were also not well tolerated, such as 23m (app K_{D}=74 μM, SF=4.5) and 23n (app K_{D}=94 μM, SF=3.5) (Table 8). With both 23m and 23n occupying solvent exposed space within the composite pocket, but not engaging in contacts with p65. Interestingly, fragment 23w (app K_{D}=26 μM, SF=12.7) showed a significant drop in stabilization in respect to 23f. Crystal soaking experiments showed the fragment engages in hydrophobic interactions with Ile46, Pro47 and Gly48 of p65. However, unlike other fragments 23w induces a conformational change in the C-terminus of the peptide shifting toward the pS45. Analysis of soaking experiment with fragment 23y, showed the tetrahydroisoquinoline ring engages in hydrophobic contacts with the p65 peptide, however, unlike 23e and 23f the bicyclic rings repulses Pro47 of p65. This provides an explanation for the poor stabilizing activity of 23y. In contrast to 2-tetrahydroquinoline (23y), 1-tetrahydroquinoline (23z) was highly tolerated eliciting CC₅₀=57 μM and app K_{D}=5.1 μM, this translated to ˜65-fold stabilization. X-ray crystallography analysis of fragment 23z, provided valuable explanation for high stabilization observed in FA assays. Specifically, the bicyclic substructure in 23z engage the p65 peptide and is positioned upwards occupying the hydrophobic roof of 14-3-3σΔC shaped by residues Ile219, Leu218, and Leu222. Further, 23z also engages in direct hydrophobic contacts with Ile46 and Pro57 of p65. Notably, the 14-3-3/p65/23z ternary complex formation results in a re-orientation of the p65 peptide leading to additional 14-3-3/p65 contacts. Fragment 23z, appears to function as a template for the additional binding of p65, increasing cooperative behavior of the ternary complex. Fragment 23z facilitates additional 14-3-3-p65 contacts by enabling the C-terminus of p65 to wrap over 23z and engage in electrostatic contacts with 14-3-3. Specifically, salt bridges are observed between Arg49/Glu14 and Arg50/Asp215 of p65_45/14-3-3, respectively. These structural features translate to high activity in FA assays, with an CC₅₀ of 57 μM and an SF of 65. The additional interactions between 14-3-3 and p65 induced by the binding of 23z lead to increased cooperativity of the ternary complex. This improved cooperative behavior, directly translates into improved stabilization of the 14-3-3/p65 complex.

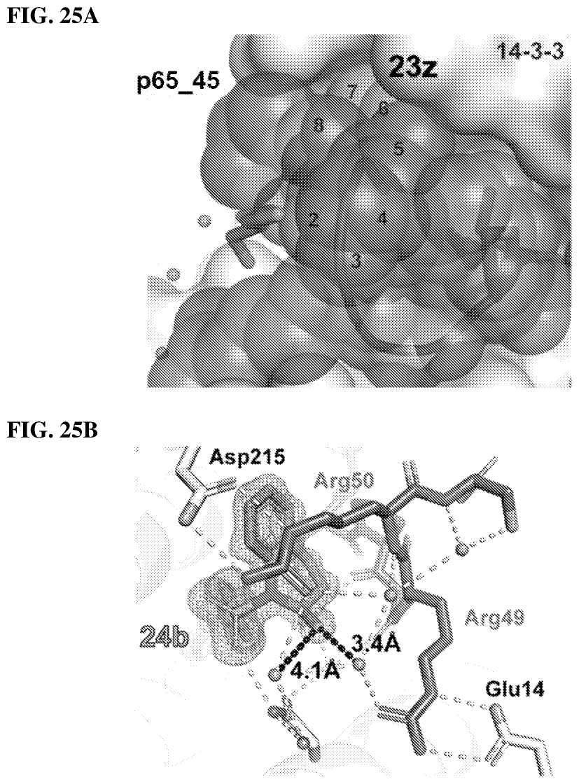

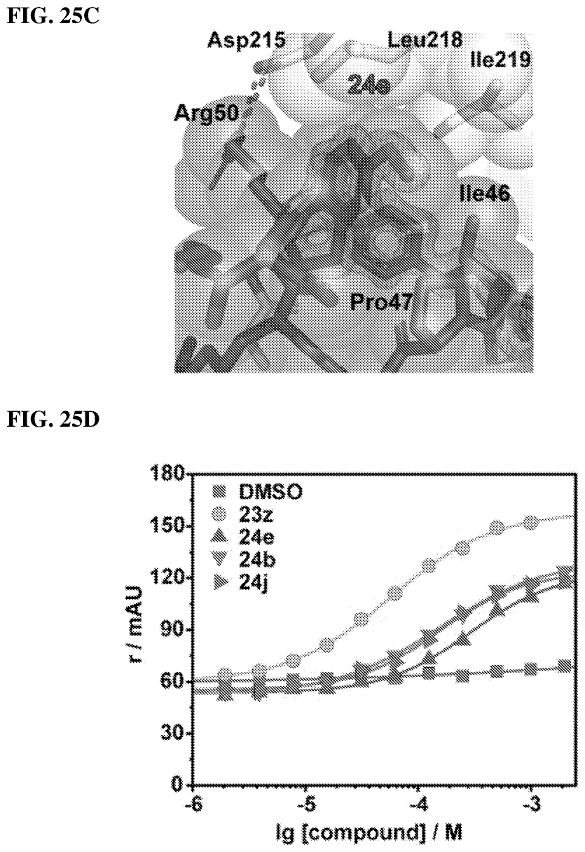

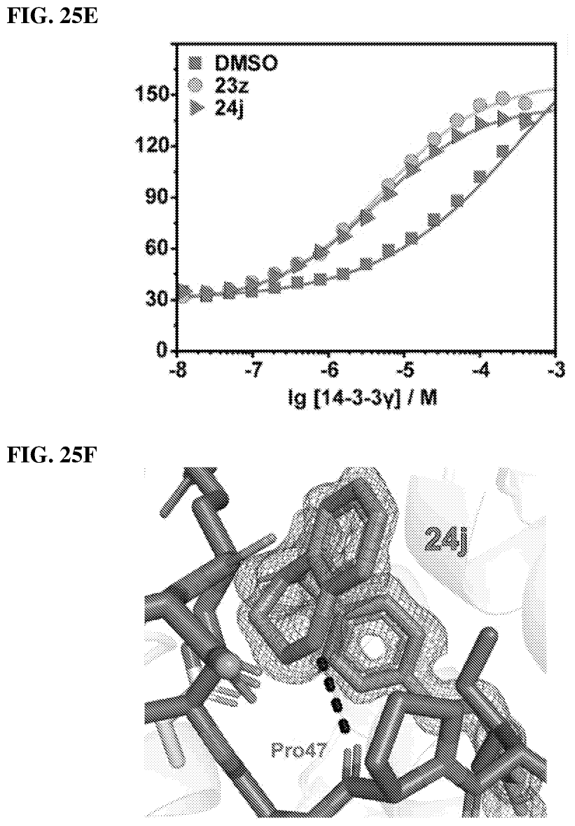

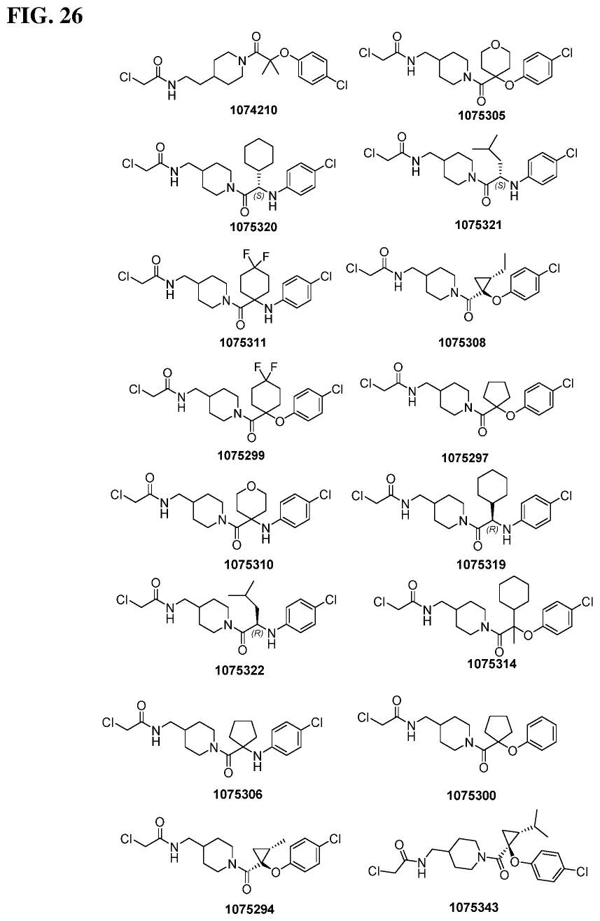

Optimization of fragment 23z. Encouraged by the high stabilizing effect of covalent fragment 23z we looked to develop a library of ten sufonylamides based on tricyclic fragment 23z. Due to the rearrangement of the p65 peptide in the presence of 23z forming a narrow and enclosed binding pocket, this limited the sites of fragment modifications to the 5-, 6- or 7-position of the tetrahydroquinoline ring (FIG. 25A). As a result, we focused on small modifications to build additional interactions with the hydrophobic patch in the roof of 14-3-3 or extension of the fragments over Ile46 of p65. Additionally, we investigated the addition of heteroatoms in the 4-position of the tetrahydroquinoline ring to engage with the backbone carbonyl or nitrogen of Pro47 or Gly48 to establish additional polar contacts. A small library of 10 analogues was synthesized using our previously mentioned sulfonamide coupling (Table 9).

TABLE 9
Exploration of structural analogs of 23z. CC₅₀: values of compound
titrations with 50 μM 14-3-3γ. K_{D,app}: values of protein titrations in
presence of 1 mM of fragment. SF: the fold-change of apparent K_{D}
in comparison to a DMSO control. All fragments bound to the
p65_45/14-3-3σΔC complex.

| | | EC₅₀ | K_{D,app} | |
|---|---|---|---|---|
| No | R | (μM) | (μM) | SF |
| 23y | 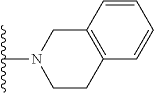 [SMILES: *N1CCc2ccccc2C1] | 180 | 450^{a} | 0.7^{a} |
| 23z | 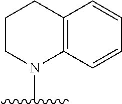 [SMILES: *N1CCCc2ccccc21] | 57 | 5.1 | 64.7 |
| 24a | 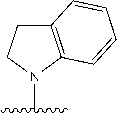 [SMILES: *N1CCc2ccccc21] | 220 | 11 | 30 |
| 24b^{a} | 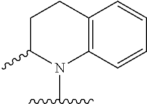 [SMILES: *N1c2ccccc2CCC1C] | 310 | 9.6 | 36 |
| 24c | 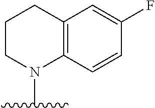 [SMILES: *N1CCCc2cc(F)ccc21] | 140 | 6.2 | 56 |
| 24d | 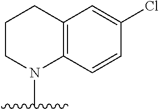 [SMILES: *N1CCCc2cc(Cl)ccc21] | 160 | 9.9 | 36 |
| 24e | 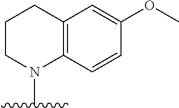 [SMILES: *N1CCCc2cc(OC)ccc21] | 120 | 10 | 34 |
| 24f | 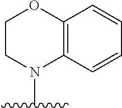 [SMILES: *N1CCOc2ccccc21] | 220 | 15 | 23 |
| 24g | 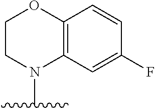 [SMILES: *N1CCOc2ccc(F)cc21] | 310 | 16 | 22 |
| 24h | 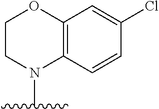 [SMILES: *N1CCOc2cc(Cl)ccc21] | 250 | 12 | 28 |
| 24i | 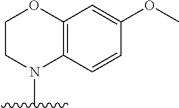 [SMILES: *N1CCOc2cc(OC)ccc21] | 110 | ND | — |
| 24j | 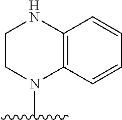 [SMILES: *N1CCNc2ccccc21] | 140 | 4.3 | 81 |

^{a} = tested as enatiomeric mixture. ND = not determined

Structural analysis of this library showed that this series of analogues retained a similar cooperative behavior as 23z, inducing additional PPI contacts. Biophysical analysis using a FA assay provided valuable insight into the SAR around fragment 23z. Analysis of the CC₅₀ concentration indicated that 23z showed the highest ternary complex formation for the with all other analogues eliciting a drop in complexation with EC₅₀ values ranging from 110-310 μM. However, significant insight into fragment stabilization was gained from analysis of app K_{D} values. Substitution of 23z for an indoline (24a) or racemic 2-methyl-tetrahydroquinoline (24b) were tolerated, however, 2-fold reduction was observed in stabilization, with app K_{D} values of 11 and 9.6 μM, respectively. Fragment 24b showed excellent electron density across the entire fragment within the X-ray crystal structure, enabling unambiguous assignment of stereochemistry. From the structure it was identified that the R-enantiomer exclusively bound with the crystal structure, suggesting that the pure R-entiomer may have significantly underestimate CC₅₀ and app K_{D} (FIG. 25B). This site may provide an excellent point for fragment extension towards the p65 peptide. Addition of a halogen or methoxy in the 6-position resulted in a reduction in stabilization, with 6-fluoro (24c), 6-chloro (24d) or 6-methoxy (24e) substituted tetrahydroquinoline fragments affording app K_{D} values of 6.2, 9.9 and 10 μM, respectively. Analysis of soaking experiments show that modifications to this position resulted in unfavorable contacts with the roof of 14-3-3 (FIG. 25D). Addition of an oxygen to the saturated 6-membered ring (24f) proved detrimental to stabilization (app K_{D}=15 μM). Further addition of halogens or methoxy groups in the 6 or 7 positions did not reestablish lost stabilization, such as 6-chloro (24h), 6-methoxy (24i) or 7-fluoro (24g). Substitution of the benzomorpholine (24f) for tetrahydroquinoxaline (24j) resulted in an improvement in stabilization with 24j eliciting an app K_{D} of 4.3, translating into the highest stabilizing effect with 81-fold stabilization (FIGS. 25D-25E). Structural analysis showed that the introduction of a nitrogen atom in the saturated ring installs an additional hydrogen bond with the backbone carbonyl of Pro47 of p65 (FIG. 25F).

These covalent fragments form a covalent tether with Lys122 within the 14-3-3/p65 composite binding pocket. Specifically, we describe the optimization of initial hit covalent fragments into a p65/14-3-3 molecular glue which elicited an 81-fold stabilization of the 14-3-3/p65 complex. Critical to this success was the use of X-ray crystallography and FA measurements to develop a robust understanding of the structural activity relationship. Direct hydrophobic engagement with p65 was the driving interaction which established complex stabilization. Hydrophobic contacts between 23z or 24j with p65 resulted in a conformational change in the 14-3-3/p65 interface with extended interactions, in turn increasing cooperativity of the ternary complex.

Observations from this research indicate that direct engagement of molecular glues with the partner peptide is significant for stabilization. Specifically, hydrophobic contacts are effective to enhance ternary complex formation and support a cooperative binding mode. Analysis of SAR results indicate that fragments which facilitate templating of the p65 peptide and promotion of additional contacts between 14-3-3 and p65 resulted in increased complex stabilization.

### Example 12: Additional Compound Characterization

Protein Expression and Purification

The 14-3-3 proteins were expressed and purified using standard protocols. In short: pPROEX HTb vectors encoding the 14-3-3σΔC (truncated C-terminus ΔC17) and 14-3-3γ isoform were transformed into BL21(DE3) cells. Protein expression was initiated with 0.4 mM IPTG at a cell density OD₆₀₀=0.8-1. The expression took place overnight at 18° C. The cells were harvested by centrifugation (10.000×g, 15 min) and resuspended in lysis buffer (50 mM Tris/HCl pH8, 300 mM NaCl, 12.5 mM imidazole, 2 mM β-mercaptoethanol). The cells were lysed with a homogenizer and the lysate was cleared via centrifugation (40.000×g, 30 min). Ni-NTA-columns were used to isolate the protein, which was washed with 10 CV lysis buffer and eluted with 250 mM imidazole (50 mM Tris/HCl pH 8, 300 mM NaCl, 250 mM imidazole, 2 mM β-mercaptoethanol). The full length 14-3-3γ was dialysis against 25 mM HEPES pH7.5, 100 mM NaCl, 10 mM MgCl₂, 0.5 mM Tris(2-carboxyethyl)phosphine) and stored at −80° C. For the 14-3-3σΔC, the His6-tag was removed by TEV protease; the TEV was removed with Ni-NTA-columns. The rest imidazole of the 14-3-3σΔC solution was removed by size exclusion chromatography (20 mM HEPES pH 7.5, 150 mM NaCl, 2 mM 3-mercaptoethanol) and stored at −80° C.

X-Ray Crystallography

Binary crystals with p65_45 peptide (Sequence: EGRSAG pS45 IPGRRS, C-terminus: amidation; N-terminus: acetylation (SEQ ID NO:9)) and 14-3-3σΔC were grown at a 14-3-3σΔC concentration of 12 mg/ml in a 1:2 ratio with the acetylated peptide in 20 mM HEPES pH7.5, 2 mM MgCl₂, 2 mM β-mercaptoethanol. This complexation mixture was incubated overnight. In a hanging drop set up the complexation mixture was mixed in 1:2 ratio with precipitation buffer (95 mM HEPES pH 7.5, 27-28% PEG400, 190 mM CaCl₂, 5% glycerol). For data acquisition, crystals were directly flash-frozen in liquid nitrogen.

Fragment soaks were performed by adding compounds in DMSO stock solutions direct to fully grown crystals with a final compound concentration of 10 mM (≤1% DMSO). The soaks were incubated for seven days prior to data acquisition. Diffraction data were measured either at P11 beamline of PetraIII (DESY campus, Hamburg, Germany) or i-03/i-24 beamline of the diamond light source (Oxford, UK) or in-house. The diffraction data were integrated with the xia2/DIALS pipeline, followed by molecular replacement with MolRep. The binary p65_45/14-3-3σΔC structure as used as a search model (PDB ID: 6QHL). Model refinement took place in iterative cycles with Coot, Refmac5 and phenix.refine. 3D structures of ligands were prepared using the fragment SMILES and elbow of the phenix suite. Figures were generated with PyMOL© (V2.0.6, Schrodinger LLC).

Fluorescence Anisotropy Assays

Complex stabilization was measured using a fluorescently labeled p65_45 peptide (FITC-βAla-EGRSAG pS₄₅ IPGRRS (residues 1-13 of SEQ ID NO:9)) at a concentration of 100 nM throughout all assays. During compound titrations, the 14-3-3γ concentration was constant 50 μM and the compound was titrated in a 1:1 dilution series. In protein titrations, 14-3-3γ was titrated in a 1:1 dilution series in the presence of 1 mM compound. The plates (Corning 384 well plates, black, round bottom, low binding) were incubated for 3 h RT prior to fluorescence anisotropy (FA) measurements with the Tecan Infinite 500 plate reader (FITC dye: excitation 485 nm, emission 535 nm). Dilution series were prepared in FA buffer (10 mM HEPES pH 7.4, 150 mM NaCl, 0.1% Tween20). All measurements were performed once.

All commercial chemicals were used as received. Reagents were used without further purification unless otherwise noted. Compounds purification and characterization. TLC analysis was performed on TLC aluminum sheets, silica gel layer, ALUGRAM SIL G UV254, 20×20 cm by MACHEREY-NAGEL. TLC plates were analyzed by UV fluorescence (254 nm). UHPLC-MS analysis was performed using UPLC Agilent Technologies 1290 Infinity coupled with Agilent Technologies 6120 Quadrupole LC/MS DAD detector. Column: ACQUITY UHPLC BEH C18 (1.7 μm) 2.1 mm×50 mm. Temperature: 40° C. Detection: DAD+MS/6120 Quadrupole. Injected volume: 1 μL. Flow: 1.2 mL/min. Solvent A: Water+0.1% Formic Acid. Solvent B: Acetonitrile+0.1% Formic Acid. Gradient: 0 min 2% B; 0.2 min 2% B; 2.0 min 98% B; 2.2 min 98% B; 2.21 min 2% B; 2.5 min 2% B. Preparative HPLC was performed using UPLC Agilent Technologies 1260 Infinity coupled with Agilent Technologies 6120 Quadrupole LC/MS. Column: Waters XBridge Prep C18 5 μm OBD 19×150 mm. Detection: DAD+MS/6120 Quadrupole. Flow: 32 mL/min. Solvent A: Water+0.1% Formic Acid. Solvent B: Acetonitrile+0.1% Formic Acid. Gradient: 0 min 77% A/23% B; 1 min 77% A/23% B; 9 min 16% A/84% B; 9.01 min 2% A/98% B; 11 min 2% A/98% B. ¹H NMR and ¹³C NMR spectra were recorded on a Bruker 300 MHz spectrometer at ambient temperature. The chemical shifts are listed in ppm on the 6=scale and coupling constants were recorded in Hertz (Hz). Chemical shifts are calibrated relative to the signals corresponding of the non-deuterated solvent (CHCl₃: 6=7.26 ppm for ¹H and 77.16 ppm for ¹³C; DMSO: 6=2.50 ppm for ¹H and 39.52 ppm for ¹³C). Abbreviations are used in the description of NMR data as follows; chemical shift (6=ppm), multiplicity (s=singlet, d=doublet, t=triplet, m=multiplet, bs=broad singlet, dd=doublet of doublets, td=triplet of doublets), coupling constant (J=Hz).

General procedure 1 (13a-u). A solution of the corresponding amine (12a-u) (0.46 mmol, 1.3 Eq.), trimethylamine (1.06 mmol, 3 Eq.) and cesium fluoride (0.03 mmol, 0.1 Eq.) in ACN (1 ml) was stirred at room temperature for 20 min. To the reaction mixture was then added a solution of 3-fluoro-4-nitrobenzaldehyde (11) (0.35 mmol, 1 Eq.) in CAN (0.5 ml). The resulting reaction was then stirred at room temperature for 24 hours. After complete consumption of the starting materials—monitored by TLC (DCM/MeOH 19:1) and UHPLC-MS the mixture was filtered, and filtrate was evaporated under reduced pressure. Compound was purified by preparative HPLC.

3-(3-methoxyazetidin-1-yl)-4-nitrobenzaldehyde (13a). Compound 13a was synthesized using general procedure 1 and 3-azetidinyl methyl ether hydrochloride (12a). The crude reaction was purified using preparative HPLC to afford the desired compound as an orange amorphous solid (26 mg, 30% yield) with a purity of 98% by UHPLC-MS. UHPLC-MS (ESI+APCI) m/z calcd. for C₁₁H₁₂N₂O₄ [M+H]⁺=237. Found: 237. Retention time: 1.29 min. ¹H NMR (300 MHz, CDCl3) δ 9.99 (s, 1H), 7.89 (d, J=8.36 Hz, 1H), 7.21 (dd, J=8.36 Hz, J=1.46 Hz, 1H), 7.10 (d, J=1.39 Hz, 1H), 4.31 (m, 1H), 4.23 (t, 2H), 3.86 (dd, J=9.69 Hz, J=3.46 Hz, 2H), 3.33 (s, 3H) ppm; ¹³C NMR (75 MHz, CDCl₃) δ 190.9, 144.8, 139.1, 138.5, 127.2, 116.8, 116.7, 69.0, 60.1 and 51.2 ppm.

3-(4-methoxypiperidin-1-yl)-4-nitrobenzaldehyde (13b). Compound 13b was synthesized using general procedure 1 and 4-methoxypiperidine (12b). The crude reaction was purified using preparative HPLC to afford the desired compound as orange amorphous solid (35 mg, 40% yield) with a purity of 99% by UHPLC-MS. UHPLC-MS (ESI+APCI) m/z calcd. for C₁₃H₁₆N₂O₄ [M+H]⁺=265. Found: 265. Retention time: 1.38 min. ¹H NMR (300 MHz, CDCl3) δ 9.99 (s, 1H), 7.82 (d, J=8.21 Hz, 1H), 7.62 (d, J=1.44 Hz, 1H), 7.44 (dd, J=8.20 Hz, J=1.51 Hz, 1H), 3.42 (m, 1H), 3.36 (s, 3H), 3.28 (m, 2H), 2.96 (m, 2H), 1.99 (m, 2H), 1.77 (m, 2H) ppm. ¹³C NMR (75 MHz, CDCl3) δ 190.7, 146.5, 145.7, 139.1, 126.6, 121.6, 121.5, 74.7, 55.6, 48.7 and 30.5 ppm.

3-(4-methylpiperidin-1-yl)-4-nitrobenzaldehyde (13c). Compound 13c was synthesized using general procedure 1 and 4-methylpiperidine (12c). The crude reaction was purified using preparative HPLC to afford the desired compound as red oil (34 mg, 40% yield) with a purity of 99% by UHPLC-MS. UHPLC-MS (ESI+APCI) m/z calcd. for C₁₃H₁₆N₂O₃ [M+H]⁺=249. Found: 249. Retention time: 1.66 min. ¹H NMR (300 MHz, CDCl₃) δ 9.99 (s, 1H), 7.80 (d, J=8.21 Hz, 1H), 7.60 (d, J=1.47 Hz, 1H), 7.40 (dd, J=8.20 Hz, J=1.53 Hz, 1H), 3.28 (d, J=12.46 Hz, 2H), 2.88 (td, J=12.27 Hz, J=2.21 Hz, 2H), 1.72 (dd, J=12.97 Hz, J=2.04 Hz, 2H), 1.54 (m, 1H), 1.38 (qd, J=12.29 Hz, J=3.73 Hz, 2H), 0.99 (d, J=6.35 Hz, 3H) ppm. ¹³C NMR (75 MHz, CDCl₃) δ 190.9, 146.7, 145.3, 139.1, 126.6, 121.5, 121.0, 51.8, 34.0, 30.4 and 21.7 ppm.

3-(3-methylpiperidin-1-yl)-4-nitrobenzaldehyde (13d). Compound 13d was synthesized using general procedure 1 and 3-methylpiperidine (12d). The crude reaction was purified using preparative HPLC to afford the desired compound as a red amorphous solid (44 mg, 50% yield) with a purity of 98% by UHPLC-MS. UHPLC-MS (ESI+APCI) m/z calcd. for C₁₃H₁₆N₂O₃ [M+H]⁺=249. Found: 249. Retention time: 1.67 min. ¹H NMR (300 MHz, CDCl3) δ 9.99 (s, 1H), 7.80 (d, J=8.12 Hz, 1H), 7.59 (d, J=1.43 Hz, 1H), 7.40 (dd, J=8.20 Hz, J=1.51 Hz, 1H), 3.21 (m, 2H), 2.81 (m, 1H), 2.52 (m, 1H), 1.84 (m, 2H), 1.71 (m, 2H), 1.06 (m, 1H), 0.91 (d, J=6.42 Hz, 3H) ppm. ¹³C NMR (75 MHz, CDCl3) δ 190.9, 146.7, 145.3, 139.1, 126.6, 124.6, 121.0, 59.1, 52.1, 32.3, 31.0, 25.2 and 19.0 ppm.

3-(3-methoxypiperidin-1-yl)-4-nitrobenzaldehyde (13e). Compound 13e was synthesized using general procedure 1 and 3-methoxypiperidine (12e). The crude reaction was purified using preparative HPLC to afford the desired compound as a red oil (37 mg, 40% yield) with a purity of 98% by UHPLC-MS. UHPLC-MS (ESI+APCI) m/z calcd. for C₁₃H₁₆N₂O₄ [M+H]⁺=265. Found: 265. Retention time: 1.41 min. ¹H NMR (300 MHz, CDCl3) δ 10.01 (s, 1H), 7.83 (d, J=8.22 Hz, 1H), 7.63 (d, J=1.43 Hz, 1H), 7.45 (dd, J=8.23 Hz, J=1.52 Hz, 1H), 3.43 (m, 2H), 3.37 (s, 3H), 3.19 (m, 1H), 2.87 (m, 1H), 2.76 (td, J=9.50 Hz, J=1.90 Hz, 1H), 2.10 (m, 1H), 1.84 (m, 1H), 1.67 (m, 1H), 1.42 (m, 1H) ppm. ¹³C NMR (75 MHz, CDCl3) δ 190.7, 146.4, 145.5, 139.2, 126.6, 122.1, 121.5, 75.4, 56.3, 55.7, 51.7, 29.5 and 23.1 ppm.

3-(4-hydroxypiperidin-1-yl)-4-nitrobenzaldehyde (13f). Compound 13f was synthesized using general procedure 1 and 4-hydroxypiperidine (12f). The crude reaction was purified using preparative HPLC to afford the desired compound as a red oil (61 mg, 68% yield) with a purity of 97% by UHPLC-MS. UHPLC-MS (ESI+APCI) m/z calcd. for C₂H₁₄N₂O₄[M+H]⁺=251. Found: 251. Retention time: 1.13 min. ¹H NMR (300 MHz, CDCl3) δ 9.98 (s, 1H), 7.81 (d, J=8.20 Hz, 1H), 7.61 (d, J=1.44 Hz, 1H), 7.44 (dd, J=8.22 Hz, J=1.52 Hz, 1H), 3.90 (m, 1H), 3.30 (m, 2H), 2.96 (m, 2H), 1.99 (m, 4H), 1.71 (m, 2H) ppm. ¹³C NMR (75 MHz, CDCl3) δ 190.6, 146.3, 145.6, 138.9, 126.4, 121.7, 121.4, 66.5, 48.7 and 33.9 ppm.

3-(4-acetylpiperazin-1-yl)-4-nitrobenzaldehyde (13g). Compound 13g was synthesized using general procedure 1 and 1-acetylpiperazine (12g). The crude reaction was purified using preparative HPLC to afford the desired compound as an orange amorphous solid (20 mg, 20% yield) with a purity of 94% by UHPLC-MS. UHPLC-MS (ESI+APCI) m/z calcd. for C₁₃H₁₅N₃O₄ [M+H]⁺=278. Found: 278. Retention time: 1.12 min. ¹H NMR (300 MHz, CDCl3) δ 10.03 (s, 1H), 7.88 (d, J=8.17 Hz, 1H), 7.65 (d, J=1.37 Hz, 1H), 7.59 (dd, J=8.18 Hz, J=1.50 Hz, 1H), 3.79 (t, 2H), 3.63 (t, 2H), 3.11 (m, 4H), 2.14 (s, 3H) ppm. ¹³C NMR (75 MHz, CDCl3) δ 190.3, 169.2, 146.9, 145.8, 139.2, 126.5, 123.9, 121.6, 51.9, 51.3, 46.1, 41.3 and 21.3 ppm.

3-[4-(dimethylamino)piperidin-1-yl]-4-nitrobenzaldehyde (13h)—formate salt. Compound 13h was synthesized using general procedure 1 and 4-(dimethylamino)piperidine (12h). The crude reaction was purified using preparative HPLC to afford the desired compound as a red oil (20 mg, 20% yield) with a purity of 95% by UHPLC-MS. UHPLC-MS (ESI+APCI) m/z calcd. for C₁₄H₁₉N₃O₃ [M+H]⁺=278. Found: 278. Retention time: 0.71 min. ¹H NMR (300 MHz, CDCl3) δ 10.02 (s, 1H), 8.47 (s, 1H, formate proton), 7.85 (d, J=8.19 Hz, 1H), 7.63 (d, J=1.44 Hz, 1H), 7.52 (dd, J=8.20 Hz, J=1.51 Hz, 1H), 6.97 (bp, 1.5H, formate proton), 3.41 (d, J=12.48 Hz, 2H), 2.90 (m, 4H), 2.53 (s, 6H), 2.06 (bp, 1H), 2.02 (bp, 1.5H), 1.81 (qd, J=12.08 Hz, J=3.90 Hz, 2H) ppm. ¹³C NMR (75 MHz, CDCl3) δ 190.5, 167.9 (formate carbon), 146.3, 146.0, 139.2, 126.5, 122.9, 121.8, 61.3, 50.9, 39.7 and 26.8 ppm.

4-nitro-3-[4-(propan-2-yl)piperazin-1-yl]benzaldehyde (13i)—formate salt. Compound 13i was synthesized using general procedure 1 and 1-isopropylpiperazine (12i). The crude reaction was purified using preparative HPLC to afford the desired compound as a red oil (48 mg, 50% yield) with a purity of 96% by UHPLC-MS. UHPLC-MS (ESI+APCI) m/z calcd. for C₁₄H₁₉N₃O₃ [M+H]⁺=278. Found: 278. Retention time: 0.71 min. ¹H NMR (300 MHz, CDCl3) δ 10.02 (s, 1H), 8.43 (s, 1H, formate proton), 7.85 (d, J=8.21 Hz, 1H), 7.69 (d, J=1.40 Hz, 1H), 7.58 (dd, J=8.22 Hz, J=1.49 Hz, 1H), 7.20 (bp, 1H), 3.30 (t, 4H), 3.15 (m, 1H), 2.97 (t, 4H), 1.22 (d, J=6.63 Hz, 6H) ppm. ¹³C NMR (75 MHz, CDCl3) δ 190.3, 167.3 (formate carbon), 146.9, 145.5, 139.3, 126.3, 123.4, 122.3, 55.6, 50.2, 47.7 and 17.4 ppm.

3-(4-ethylpiperazin-1-yl)-4-nitrobenzaldehyde (13j)—formate salt. Compound 13j was synthesized using general procedure 1 and 1-ethylpiperazine (12j). The crude reaction was purified using preparative HPLC to afford the desired compound as a red oil (25 mg, 30% yield) with a purity of 99% by UHPLC-MS. UHPLC-MS (ESI+APCI) m/z calcd. for C₁₃H₁₇N₃O₃ [M+H]⁺=264. Found: 264. Retention time: 0.63-0.66 min. ¹H NMR (300 MHz, CDCl3) δ 10.02 (s, 1H), 8.41 (s, 0.3H, formate proton), 7.84 (d, J=8.05 Hz, 1H), 7.65 (s, 1H), 7.5 (d, J=8.20 Hz, 1H), 6.01 (bp, 1H, formate proton), 3.22 (d, J=3.65 Hz, 4H), 3.76 (d, J=3.24 Hz, 4H), 2.62 (m, 2H), 1.17 (m, 3H) ppm. ¹³C NMR (75 MHz, CDCl3) δ 190.5, 167.1 (formate carbon), 146.5, 145.8, 139.2, 126.4, 122.9, 121.7, 52.0, 51.1, 50.7 and 11.1 ppm.

3-[4-(2-methylpropyl)piperazin-1-yl]-4-nitrobenzaldehyde (13k)—formate salt. Compound 13k was synthesized using general procedure 1 and 1-isobutylpiperazine (12k). The crude reaction was purified using preparative HPLC to afford the desired compound as a red oil (31 mg, 30% yield) with a purity of 99%. UHPLC-MS (ESI+APCI) m/z calcd. for C₁₅H₂₁N₃O₃ [M+H]⁺=292. Found: 292. Retention time: 0.76 min. ¹H NMR (300 MHz, CDCl3) δ 10.01 (s, 1H), 8.37 (s, 0.3H, formate proton), 7.83 (d, J=8.19 Hz, 1H), 7.64 (d, J=1.42 Hz, 1H), 7.50 (dd, J=8.20 Hz, J=1.50 Hz, 1H), 6.93 (bp, 0.6H, formate proton), 3.18 (t, 4H), 2.67 (t, 4H), 2.27 (d, J=7.30 Hz, 2H), 1.86 (m, 1H), 0.93 (d, J=6.59 Hz, 6H) ppm. ¹³C NMR (75 MHz, CDCl3) δ 190.6, 166.5 (formate carbon), 146.2, 146.0, 139.2, 126.5, 122.4, 121.6, 66.2, 52.9, 50.8, 25.1 and 20.9 ppm.

3-(3-hydroxypiperidin-1-yl)-4-nitrobenzaldehyde (13l). Compound 13l was synthesized using general procedure 1 and 3-hydroxypiperidine (12l). The crude reaction was purified using preparative HPLC to afford the desired compound as a red oil (55 mg, 61% yield) with a purity of 91% by UHPLC-MS. UHPLC-MS (ESI+APCI) m/z calcd. for C₂H₁₄N204 [M+H]⁺=251. Found: 251. Retention time: 1.18 min. ¹H NMR (300 MHz, CDCl3) δ 10.00 (s, 1H), 7.83 (d, J=8.19 Hz, 1H), 7.64 (d, J=1.49 Hz, 1H), 7.50 (dd, J=8.21 Hz, J=1.56 Hz, 1H), 3.94 (m, 1H), 3.24 (dd, J=11.74 Hz, J=2.85 Hz, 1H), 3.01 (m, 1H), 2.41 (bp, 1H), 1.87 (m, 2H), 1.65 (m, 2H) ppm. ¹³C NMR (75 MHz, CDCl3) δ 190.5, 146.5, 146.1, 139.0, 126.3, 122.4, 122.1, 65.7, 58.2, 52.4, 31.4 and 21.6 ppm.

1-(5-formyl-2-nitrophenyl)piperidine-4-carbonitrile (13m). Compound 13m was synthesized using general procedure 1 and 4-cyanopiperidine (12m). The crude reaction was purified using preparative HPLC to afford the desired compound as a red oil (20 mg, 22% yield) with a purity of 95% by UHPLC-MS. UHPLC-MS (ESI+APCI) m/z calcd. for C₁₃H₁₃N₃O₃ [M+H]⁺=260. Found: 260. Retention time: 1.33 min. ¹H NMR (300 MHz, CDCl3) δ 10.03 (s, 1H), 7.87 (d, J=8.20 Hz, 1H), 7.67 (d, J=1.45 Hz, 1H), 7.58 (dd, J=8.19 Hz, J=1.54 Hz, 1H), 3.29 (m, 2H), 3.09 (m, 2H), 2.88 (m, 1H), 2.06 (m, 4H) ppm. 13C NMR (75 MHz, CDCl₃) δ 190.3, 146.9, 146.2, 139.2, 126.4, 123.7, 121.9, 120.9, 50.0, 28.6 and 25.7 ppm.

4-nitro-3-[4-(pyrrolidin-1-yl)piperidin-1-yl]benzaldehyde (13n)—formate salt. Compound 13n was synthesized using general procedure 1 and using 4-(1-pyrrolidinyl)piperidine (12n). The crude reaction was purified using preparative HPLC to afford the desired compound as a red oil (40 mg, 40% yield) with a purity 97% by UHPLC-MS. UHPLC-MS (ESI+APCI) m/z calcd. for C₁₆H₂₁N₃O₃ [M+H]⁺=304. Found: 304. Retention time: 0.75 min. ¹H NMR (300 MHz, CDCl3) δ 10.00 (s, 1H), 8.47 (s, 1H, formate proton), 7.83 (d, J=8.20 Hz, 1H), 7.62 (d, J=1.39 Hz, 1H), 7.51 (dd, J=8.21 Hz, J=1.46 Hz, 1H), 6.74 (bp, 2H, formate proton), 3.39 (d, J=12.60 Hz, 2H), 3.14 (m, 4H), 2.93 (m, 3H), 2.11 (bp, 1H), 2.07 (bp, 1H), 1.99 (m, 7H) ppm. ¹³C NMR (75 MHz, CDCl3) δ 190.5, 167.8 (formate carbon), 146.2, 145.7, 139.2, 126.5, 122.8, 122.1, 60.3, 50.4, 49.8, 28.3 and 23.4 ppm.

3-[4-(2-methoxyethyl)piperazin-1-yl]-4-Nitrobenzaldehyde (13o)—formate salt. Compound 13o was synthesized using general procedure 1 and 1-(2-methoxyethyl) piperazine (12o). The crude reaction was purified using preparative HPLC to afford the desired compound as a red oil (30 mg, 30% yield) with a purity of 99% by UHPLC-MS. UHPLC-MS (ESI+APCI) m/z calcd. for C₁₄H₁₉N₃O₄ [M+H]⁺=294. Found: 294. Retention time: 0.68 min. ¹H NMR (300 MHz, CDCl₃) δ 10.01 (s, 1H), 8.35 (s, 0.3H, formate proton), 7.82 (d, J=8.19 Hz, 1H), 7.62 (d, J=1.43 Hz, 1H), 7.51 (dd, J=8.19 Hz, J=1.50 Hz, 1H), 5.87 (bp, 0.6H, formate proton), 3.56 (t, 2H), 3.36 (s, 3H), 3.19 (t, 4H), 2.74 (m, 6H) ppm. ¹³C NMR (75 MHz, CDCl₃) δ 190.5, 166.3 (formate carbon), 146.3, 145.9, 139.2, 126.4, 122.5, 121.5, 69.6, 58.9, 57.5, 53.0 and 50.8 ppm.

3-(4-methyl-1,4-diazepan-1-yl)-4-nitrobenzaldehyde (13p)—formate salt. Compound 13p was synthesized using general procedure 1 and 1-methyl-1,4-diazepane (12p). The crude reaction was purified using preparative HPLC to afford the desired compound as a red amorphous solid (35 mg, 40% yield) with a purity of 98% by UHPLC-MS. UHPLC-MS (ESI+APCI) m/z calcd. for C₁₃H₁₇N₃O₃ [M+H]⁺=264. Found: 264. Retention time: 0.64 min. ¹H NMR (300 MHz, CDCl3) δ 9.99 (s, 1H), 8.38 (s, 1H, formate proton), 8.20 (bp, 1.5H, formate proton), 7.79 (d, J=8.24 Hz, 1H), 7.57 (d, J=1.32 Hz, 1H), 7.37 (dd, J=8.23 Hz, J=1.34 Hz, 1H), 3.60 (t, 2H), 3.34 (t, 2H), 3.06 (t, 2H), 2.99 (t, 2H), 2.58 (s, 3H), 2.15 (m, 2H) ppm. ¹³C NMR (75 MHz, CDCl3) δ 190.7, 167.3 (formate carbon), 145.4, 143.5, 138.9, 127.0, 120.3, 119.9, 56.6, 56.4, 51.7, 50.0, 45.0 and 25.7 ppm.

3-(morpholin-4-yl)-4-nitrobenzaldehyde (13q). Compound 13q was synthesized using general procedure 1 and morpholine (12q). The crude reaction was purified using preparative HPLC to afford the desired compound as a red amorphous solid (25 mg, 30% yield) with a purity of 92% by UHPLC-MS. UHPLC-MS (ESI+APCI) m/z calcd. for C₁₁H₁₂N₂O₄ [M+H]+=237. Found: 237. Retention time: 1.22 min. ¹H NMR (300 MHz, CDCl3) δ 10.03 (s, 1H), 7.85 (d, J=8.16 Hz, 1H), 7.64 (d, J=1.45 Hz, 1H), 7.55 (dd, J=8.18 Hz, J=1.51 Hz, 1H), 3.84 (m, 4H), 3.11 (m, 4H) ppm. ¹³C NMR (75 MHz, CDCl3) δ 190.3, 146.3, 145.7, 139.0, 126.3, 122.9, 120.9, 66.4 and 51.5 ppm.

3-[4-(2-hydroxyethyl)piperidin-1-yl]-4-nitrobenzaldehyde (13r). Compound 13r was synthesized using general procedure 1 and 4-piperidineethanol (12r). The crude reaction was purified using preparative HPLC to afford the desired compound as a red oil (20 mg, 20% yield) with a purity of 98% by UHPLC-MS. UHPLC-MS (ESI+APCI) m/z calcd. for C₁₄H₁₈N₂O₄ [M+H]⁺=279. Found: 279. Retention time: 1.30 min. ¹H NMR (300 MHz, CDCl3) δ 10.01 (s, 1H), 7.82 (d, J=8.20 Hz, 1H), 7.60 (d, J=1.41 Hz, 1H), 7.42 (dd, J=8.21 Hz, J=1.49 Hz, 1H), 3.73 (t, 2H), 3.31 (d, J=12.39 Hz, 2H), 2.89 (td, J=12.18 Hz, J=2.01 Hz, 2H), 1.80 (d, J=12.86 Hz, 2H), 1.61 (m, 3H), 1.43 (qd, J=12.37 Hz, J=3.82 Hz, 3H) ppm. ¹³C NMR (75 MHz, CDCl3) δ 190.9, 146.7, 145.5, 139.1, 126.6, 121.4, 121.3, 60.3, 51.9, 39.1, 32.1 and 31.9 ppm.

4-nitro-3-(1,2,3,4-tetrahydroisoquinolin-2-yl)benzaldehyde (13s). Compound 13s was synthesized using general procedure 1 and 1,2,3,4-tetrahydroisochinoline (12s). The crude reaction was purified using preparative HPLC to afford the desired compound as a red oil (31 mg (31% yield) with a purity of 92% by UHPLC-MS. UHPLC-MS (ESI+APCI) m/z calcd. for C₁₆H₁₄N₂O₃ [M+H]⁺=283. Found: 283. Retention time: 1.64 min. ¹H NMR (300 MHz, CDCl3) δ 10.03 (s, 1H), 7.89 (d, J=8.22 Hz, 1H), 7.69 (d, J=1.41 Hz, 1H), 7.42 (dd, J=8.23 Hz, J=1.49 Hz, 1H), 7.20 (m, 3H), 7.13 (m, 1H), 4.36 (s, 2H), 3.45 (t, 2H), 3.02 (t, 2H) ppm. ¹³C NMR (75 MHz, CDCl3) δ 190.9, 145.5, 143.9, 139.1, 134.3, 133.1, 128.7, 127.2, 126.9, 126.4, 126.3, 120.4, 119.8, 52.1, 49.5 and 28.7 ppm.

4-nitro-3-(2-phenylpyrrolidin-1-yl)benzaldehyde (13t). Compound 13t was synthesized using general procedure 1 and 2-phenylpyrrolidine (12t). The crude reaction was purified using preparative HPLC to afford the desired compound as a red amorphous solid (36 mg, 35% yield) with a purity of 96% by UHPLC-MS. UHPLC-MS (ESI+APCI) m/z calcd. for C₁₇H₁₆N₂O₃ [M+H]⁺=297. Found: 297. Retention time: 1.67 min. ¹H NMR (300 MHz, CDCl3) δ 9.79 (s, 1H), 7.80 (d, J=8.33 Hz, 1H), 7.31 (s, 1H), 7.30 (d, J=2.30 Hz, 2H), 7.23 (m, 2H), 7.14 (dd, J=8.34 Hz, J=1.46 Hz, 1H), 4.93 (t, 1H), 3.82 (m, 1H), 2.99 (m, 1H), 2.55 (m, 1H), 2.13 (m, 1H), 1.92 (m, 2H) ppm. 13C NMR (75 MHz, CDCl3) δ 191.2, 142.2, 141.6, 140.8, 138.4, 129.0, 127.6, 127.2, 126.0, 119.3, 115.8, 64.7, 53.1, 37.5 and 25.7 ppm.

3-[methyl(oxan-4-yl)amino]-4-nitrobenzaldehyde (13u). Compound 13u was synthesized using general procedure 1 and N-methyl-N-tetrahydro-2H-pyran-4-ylamine (12u). The crude reaction was purified using preparative HPLC to afford the desired compound as a red amorphous solid (17 mg, 20% yield) with a purity of 99% by UHPLC-MS. UHPLC-MS (ESI+APCI) m/z calcd. for _{C13H16N204} [M+H]⁺=265. Found: 265. Retention time: 1.34 min. ¹H NMR (300 MHz, CDCl3) δ 10.01 (s, 1H), 7.80 (d, J=8.21 Hz, 1H), 7.61 (d, J=1.33 Hz, 1H), 7.39 (dd, J=8.22 Hz, J=1.46 Hz, 1H), 4.04 (dd, J=11.45 Hz, J=4.33 Hz, 2H), 3.42 (m, 3H), 2.77 (s, 3H), 1.90 (qd, J=12.73 Hz, J=4.53 Hz, 2H), 1.73 (dd, J=12.58 Hz, J=1.95 Hz, 2H) ppm. ¹³C NMR (75 MHz, CDCl3) δ 190.8, 145.8, 145.0, 138.8, 126.8, 121.6, 120.5, 67.3, 60.0, 34.0 and 29.9 ppm.

General procedure 2 (16a-k). A suspension of 2-fluoro-5-pyridinecarboxyaldehyde (14) (0.39 mmol, 1 Eq.), the corresponding imidazole (15a-e) or benimidazole (15f-k) (0.44 mmol, 1.1 Eq.), triethylamine (1.20 mmol, 3 Eq.) and cesium fluoride (0.04 mmol, 0.1 Eq.) in 2 ml of THF was stirred at room temperature for 48 hours. After complete consumption of the starting materials—monitored by TLC (CHCl₃/MeOH 9:1) and UHPLC-MS—the mixture was concentrated under reduced pressure. Compound was purified by preparative HPLC.

6-(1H-imidazol-1-yl)pyridine-3-carbaldehyde (16a). Compound 16a was synthesized using general procedure 2 and imidazole (15a). The crude reaction was purified using preparative HPLC to afford the desired compound as a yellow oil (42 mg, 72% yield) with a purity of 99% by UHPLC-MS. UHPLC-MS (ESI+APCI) m/z calcd. for C9H7N30 [M+H]⁺=174. Found: 174. Retention time: 0.25 min. ^{1H} NMR (300 MHz, CDCl3) δ 10.09 (s, 1H), 8.93 (d, J=1.6 Hz, 1H), 8.47 (s, 1H), 8.31 (dd, J=2.2 Hz, J=8.49 Hz, 1H), 7.70 (t, 1H), 7.50 (d, J=8.48 Hz, 1H), 7.23 (m, 1H) ppm. ¹³C NMR (75 MHz, CDCl3) δ 189.0, 152.5, 152.2, 138.9, 135.4, 131.5, 129.9, 116.1, 112.2 ppm.

6-(4-chloro-1H-imidazol-1-yl)pyridine-3-carbaldehyde (16b). Compound 16b was synthesized using general procedure 2 and 4-chloroimidazole (15b). The crude reaction was purified using preparative HPLC to afford the desired compound as a white solid (16.8 mg, 17% yield) with a purity of 99% by UHPLC-MS. UHPLC-MS (ESI+APCI) m/z calcd. for C₉H₆ClN₃O [M+H]⁺=208. Found: 208. Retention time: 1.06 min. ¹H NMR (300 MHz, CDCl₃) δ 10.12 (s, 1H), 8.94 (d, J=1.66 Hz, 1H), 8.36 (t, 1.5H), 8.33 (d, J=2.18 Hz, 0.5H), 7.62 (d, J=1.57 Hz, 1H), 7.47 (d, J=8.48 Hz, 1H) ppm. ¹³C NMR (75 MHz, CDCl₃) δ 188.6, 152.2, 139.0, 133.7, 130.1, 111.9, 111.7 ppm.

6-(4-acetyl-1H-imidazol-1-yl)pyridine-3-carbaldehyde (16c). Compound 16c was synthesized using general procedure 2 and 4-acetylimidazole (15d). The crude reaction was purified using preparative HPLC to afford the desired compound as a white solid (18 mg, 21% yield) with a purity of 90% by UHPLC-MS. UHPLC-MS (ESI+APCI) m/z calcd. for C₁₁H₉₃N₃O₂ [M+H]⁺=216. Found: 216. Retention time: 0.86 min. ¹H NMR (300 MHz, CDCl3): δ 10.14 (s, 1H), 8.98 (d, J=2.07 Hz, 1H), 8.49 (d, J=1.26 Hz, 1H), 8.38 (dd, J=2.17 Hz, J=8.46 Hz, 1H), 8.33 (d, J=1.30 Hz, 1H), 7.58 (d, J=8.43 Hz, 1H), 2.63 (s, 3H) ppm. ¹³C NMR (75 MHz, CDCl3) δ 194.2, 188.7, 152.3, 151.5, 143.9, 139.4, 135.5, 130.7, 119.4, 112.6, 26.9 ppm.

6-(2-bromo-1H-imidazol-1-yl)pyridine-3-carbaldehyde (16d). Compound 16d was synthesized using general procedure 2 and 1-bromoimidazole (15d). The crude reaction was purified using preparative HPLC to afford the desired compound as a white solid (31 mg, 25% yield) with a purity of 100% by UHPLC-MS. UHPLC-MS (ESI+APCI) m/z calcd. for C₉H₆BrN₃O [M+H]⁺=252. Found: 252 and 254. Retention time: 0.92 min. ¹H NMR (300 MHz, CDCl3) δ 10.17 (s, 1H), 9.04 (d, J=2.11 Hz, 1H), 8.38 (dd, J=2.20 Hz, J=8.38 Hz, 1H), 7.89 (d, J=8.38 Hz, 1H), 7.62 (d, J=1.61 Hz, 1H), 7.19 (d, J=1.60 Hz, 1H), ppm. ¹³C NMR (75 MHz, CDCl3) δ 188.8, 152.4, 151.7, 138.3, 130.5, 122.5, 118.4, 116.9 ppm.

6-{5-[(2R)-1-(cyclopropylmethyl)pyrrolidin-2-yl]-1H-imidazol-1-yl}pyridine-3-carbaldehyde (16e). Compound 16e was synthesized using general procedure 2 and (R)-2-(1-(cyclopropylmethyl) pyrrolidin-2-yl)-1H-imidazole (15e). The crude reaction was purified using preparative HPLC to afford the desired compound as a yellow oil (11 mg, 10% yield) with a purity of 93% by UHPLC-MS. UHPLC-MS (ESI+APCI) m/z calcd. for C₁₇H2₀N40 [M+H]⁺=297. Found: 297. Retention time: 0.82 min. ¹H NMR (300 MHz, CDCl₃) δ 10.15 (s, 1H), 9.08 (d, J=1.45 Hz, 1H), 8.50 (s, 1H), 8.35 (dd, J=8.37 Hz, J=2.20 Hz, 1H), 7.76 (d, J=8.38 Hz, 1H), 7.37 (d, J=1.41 Hz, 1H), 7.16 (d, J=1.32 Hz, 1H), 4.87 (bp, 1H), 3.39 (m, 1H), 3.13 (bp, 1H), 2.51 (d, J=6.69 Hz, 2H), 2.37 (m, 2H), 2.12 (m, 4H), 0.91 (m, 1H), 0.43 (d, J=7.82 Hz, 2H), 0.02 (m, 2H) ppm. ¹³C NMR (75 MHz, CDCl₃) δ 189.2, 153.6, 152.3, 138.5, 130.3, 129.3, 119.7, 117.8, 60.6, 57.5, 53.0, 30.7, 22.7, 8.4, 4.1 and 3.9 ppm.

6-(1H-1,3-benzodiazol-1-yl)pyridine-3-carbaldehyde (16f). Compound 16f was synthesized using general procedure 2 and benzimidazole (15f). The crude reaction was purified using preparative HPLC to afford the desired compound as an off-white amorphous solid (61 mg, 59% yield) with a purity of 99% by UHPLC-MS. UHPLC-MS (ESI+APCI) m/z calcd. for C₁₃H₉N₃O [M+H]⁺=224. Found: 224. Retention time: 1.10 min. ¹H NMR (300 MHz, CDCl3) δ 10.13 (s, 1H), 9.04 (s, 1H), 8.72 (s, 1H), 8.37 (d, J=8.44 Hz, 1H), 8.21 (d, J=7.79 Hz, 1H), 7.87 (d, J=7.39 Hz, 1H), 7.75 (d, J=8.47 Hz, 1H), 7.42 (m, 2H) ppm. 13C NMR (75 MHz, CDCl3) δ 188.7, 153.1, 152.4, 144.4, 140.8, 238.5, 131.5, 129.1, 124.7, 123.9, 120.6, 113.3 and 113.2 ppm.

6-(6-fluoro-1H-1,3-benzodiazol-1-yl)pyridine-3-carbaldehyde/6-(5-fluoro-1H-1,3-benzodiazol-1-yl)pyridine-3-carbaldehyde (2:1 mixture) (16g). Compound 16g was synthesized using general procedure 2 and 5-fluoro-1H-benzo[d]imidazole (15g). The crude reaction was purified using preparative HPLC to afford the desired compound as a brown amorphous solid (23 mg, 20% yield) with a purity of 96% by UHPLC-MS as a mixture the two structural isomers in ratio 2:1. UHPLC-MS (ESI+APCI) m/z calcd. for C₁₃H₈FN₃O [M+H]⁺=242. Found: 242. Retention time: 1.18 min. ¹H NMR (300 MHz, CDCl3) δ 10.13 (s, 1.5H, major isomer), 9.05 (s, 1H, minor isomer), 9.04 (s, 1H, major isomer), 8.67 (s, 1H, minor isomer), 8.62 (s, 1H, major isomer), 8.37 (dd, J=8.53 Hz, J=2.17 Hz, 1.5H, major isomer), 8.23 (dd, J=9.02 Hz, J=4.72 Hz, 0.5H, minor isomer), 8.03 (dd, J=9.27 Hz, J=2.45 Hz, 1H, major isomer), 7.78 (dd, J=8.84 Hz, J=4.95 Hz, 1H, major isomer), 7.71 (d, J=8.43 Hz, 1H, minor isomer), 7.69 (d, J=8.52 Hz, 1.3H, major isomer), 7.52 (dd, J=8.83 Hz, J=2.47 Hz, 0.5H, minor isomer), 7.15 (qd, J=10.12 Hz, J=2.46 Hz, 1.5H, major isomer) ppm. ¹³C NMR (75 MHz, CDCl3) δ 188.9, 162.3, 159.1, 153.2, 152.5, 145.6, 145.4, 142.1, 141.1, 138.7, 132.1, 131.9, 129.5, 128.4, 121.5, 121.4, 114.7, 114.6, 113.1, 112.6, 112.3, 106.9, 106.6, 101.6, 101.2 ppm.

6-(5-chloro-1H-1,3-benzodiazol-1-yl)pyridine-3-carbaldehyde (16h). Compound 16h was synthesized using general procedure 2 and 5-Chlorobenzimidazole (15h). The crude reaction was purified using preparative HPLC to afford the desired compound as a white solid (9 mg, 10% yield) with a purity 97% by UHPLC-MS. UHPLC-MS (ESI+APCI) m/z calcd. for C₁₃H₈ClN₃O [M+H]⁺=258. Found: 258. Retention time: 1.27 min. ¹H NMR (300 MHz, CDCl3) δ 10.15 (s, 1H), 9.07 (s, 1H), 8.70 (s, 0.2H, impurity), 8.67 (s, 1H), 8.39 (d, J=8.13 Hz, 1H), 8.34 (s, 1H), 8.21 (d, J=8.77 Hz, 1H, impurity), 7.85 (s, 0.3H, impurity), 7.68 (d, J=8.61 Hz, 1H), 7.62 (d, J=8.51 Hz, 1H), 7.38 (d, J=8.31 Hz, 1H) ppm. 13C NMR (75 MHz, CDCl3) δ 188.9, 153.1, 152.6, 143.2, 141.3, 138.9, 132.4, 130.9, 129.7, 125.4, 124.9, 121.5, 120.6, 114.8, 114.4, 113.4 ppm.

6-{1H-imidazo[4,5-c]pyridin-1-yl}pyridine-3-carbaldehyde/6-{3H-imidazo[4,5-c]pyridin-3-yl}pyridine-3-carbaldehyde (1:1 mixture) (16i). Compound 16i was synthesized using general procedure 2 and 3H-imidazo[4,5-c] pyridine (15i). The crude reaction was purified using preparative HPLC to afford the desired compound as a yellow amorphous solid (8 mg, 10% yield) with a purity 98% by UHPLC-MS as a mixture of the two structural isomers in ratio 1:1. UHPLC-MS (ESI+APCI) m/z calcd. for C₁₂H₈N₄O [M+H]⁺=225. Found: 225. Retention time: 0.66 min. ¹H NMR (300 MHz, CDCl₃) δ 10.17 (s, 2H), 9.70 (d, J=0.93 Hz, 1H), 9.22 (d, J=0.89 Hz, 1H), 9.11 (dd, J=2.15 Hz, J=0.52 Hz, 1H), 9.10 (dd, J=2.18 Hz, J=0.52 Hz, 1H), 8.80 (s, 1H), 8.74 (s, 1H), ), 8.62 (d, J=2.82 Hz, 1H), 8.61 (d, J=2.66 Hz, 1H), 8.46 (dd, J=2.15 Hz, J=0.99 Hz, 1H), 8.43 (dd, J=2.16 Hz, J=0.96 Hz, 1H), 8.16 (dd, J=5.70 Hz, J=0.99 Hz, 1H), 7.82 (d, J=1.00 Hz, 0.5H), 7.80 (d, J=0.74 Hz, 1H), 7.78 (s, 1H), 7.75 (s, 0.5H) ppm. ¹³C NMR (75 MHz, CDCl₃) δ188.8, 152.8, 152.7, 152.6, 150.0, 144.5, 143.9, 143.9, 143.1, 141.9, 141.7, 139.1, 139.1, 137.1, 136.8, 130.0, 129.9, 129.7, 115.5, 113.6, 113.2, and 108.9 ppm.

6-(5-methoxy-2-methyl-1H-1,3-benzodiazol-1-yl)pyridine-3-carbaldehyde/6-(6-methoxy-2-methyl-1H-1,3-benzodiazol-1-yl)pyridine-3-carbaldehyde (1:1 mixture) (16j). Compound 16j was synthesized using general procedure 2 and 2-methyl-5-methoxybenzimidazole (15j). The crude reaction was purified using preparative HPLC to afford the desired compound as a yellow amorphous solid (8 mg, 6% yield) with a purity 85% by UHPLC-MS as a mixture of the two structural isomers in ratio 1:1. UHPLC-MS (ESI+APCI) m/z calcd. for C₁₅H₁₃N₃O₂ [M+H]⁺=268. Found: 268. Retention time: 0.91 min. ¹H NMR (300 MHz, CDCl3) δ 10.20 (s, 1H), 10.19 (s, 1H), 9.14 (d, J=2.23 Hz, 1H), 9.12 (d, J=2.21 Hz, 1H), 8.43 (dd, J=8.30 Hz, J=3.93 Hz, 1H), 8.43 (dd, J=8.30 Hz, J=2.28 Hz, 1H), 8.43 (dd, J=8.31 Hz, J=3.94 Hz, 1H), 7.66 (d, J=8.14 Hz, 1H), 7.62 (s, 1H), 7.41 (d, J=8.88 Hz, 1H), 7.25 (s, 1H), 7.00 (d, J=2.30 Hz, 1H), 6.96 (d, J=2.40 Hz, 0.5H), 6.93 (d, J=2.38 Hz, 1H), 6.90 (d, J=2.42 Hz, 0.5H), 3.88 (s, 3H), 3.83 (s, 3H), 2.77 (s, 3H), 2.73 (s, 3H) ppm. ¹³C NMR (75 MHz, CDCl3) δ 189.1, 157.2, 151.8, 150.5, 138.6, 138.5, 134.8, 130.1, 130.0, 128.4, 119.8, 119.2, 95.4, 56.0, 55.9, 16.1 and 15.9 ppm.

6-(2-chloro-5-methoxy-1H-1,3-benzodiazol-1-yl)pyridine-3-carbaldehyde/6-(2-chloro-6-methoxy-1H-1,3-benzodiazol-1-yl)pyridine-3-carbaldehyde (1:1 mixture) (16k). Compound 16k was synthesized using general procedure 2 and 2-chloro-5-methoxy-1H-1,3-benzodiazole (15k). The crude reaction was purified using preparative HPLC to afford the desired compound as a white amorphous solid (37.5 mg, 27% yield) with a purity of 94% by UHPLC-MS. UHPLC-MS (ESI+APCI) m/z calcd. for C₁₄H₁₀ClN₃O₂ [M+H]⁺=288. Found: 288. Retention time: 1.27 min. ¹H NMR (300 MHz, CDCl3) evidenced the 1:1 mixture. δ 10.23 (s, 1H), 10.21 (s, 1H), 9.18 (d, J=2.12 Hz, 1H), 9.15 (d, J=2.14 Hz, 1H), 8.45 (dd, J=2.26 Hz, J=8.30 Hz, 1H), 8.44 (dd, J=2.26 Hz, J=8.35 Hz, 1H), 7.80 (d, J=8.33 Hz, 1H), 7.79 (d, J=8.28 Hz, 1H), 7.63 (d, J=8.85 Hz, 1H), 7.63 (d, J=8.85 Hz, 1H), 7.53 (d, J=9.00 Hz, 1H), 7.23 (d, J=2.44 Hz, 1H), 7.10 (d, J=2.40 Hz, 1H), 6.99 (t, 1H), 6.96 (t, 1H), 3.88 (s, 4H), 3.83 (s, 3H) ppm. ¹³C NMR (75 MHz, CDCl3) δ 189.3, 152.4, 138.9, 138.79, 130.9, 130.8, 121.0, 120.6, 120.5, 114.4, 113.3, 112.6, 102.4, 96.0, 56.1 ppm.

General procedure 3 (19a-u). To a solution of 4-carboxybenzaldehyde (0.32 mmol, 1 Eq.) in DMF (1.5 ml) were added DIPEA (1.28 mmol, 4 Eq.) and HATU (0.35 mmol, 1.1 Eq.). The reaction mixture was stirred at room temperature for 3 hours, followed by addition of the corresponding amine (or amine HCl salt) (0.38 mmol, 1.2 Eq.). After complete consumption of the starting materials—monitored by TLC (Cyclohexane/Ethyl acetate 1:3) and UHPLC-MS—the reaction mixture was diluted with 1.5 m1 of NaHCO₃ saturated solution and extracted with DCM (3×2 ml). The organic layer was then washed with brine (3×6 ml). After separation, the organic layer was dried over magnesium sulfate and concentrated under reduced pressure. The crude reaction mixture was purified by preparative HPLC.

4-(3-methoxyazetidine-1-carbonyl)benzaldehyde (19a). Compound 19a was synthesized using general procedure 3 and 3-azetidinyl methyl ether hydrochloride (18a). The crude reaction was purified using preparative HPLC to afford the desired compound as a yellow oil (35 mg, 50% yield) with a purity of 99% by UHPLC-MS. UHPLC-MS (ESI+APCI) m/z calcd. for C₁₂H₁₃NO₃ [M+H]⁺=220. Found: 220. Retention time: 0.93 min. ¹H NMR (300 MHz, CDCl3) δ 10.05 (s, 1H), 7.92 (d, J=8.19 Hz, 2H), 7.76 (d, J=8.22 Hz, 2H), 4.38 (m, 2H), 4.26 (m, 1H), 4.13 (m, 2H), 3.31 (s, 3H) ppm. ¹³C NMR (75 MHz, CDCl3) δ 191.3, 169.0, 138.3, 137.6, 129.5, 128.2, 69.1, 59.9, 56.0 and 55.8 ppm.

4-(3-hydroxypiperidine-1-carbonyl)benzaldehyde (19b). Compound 19b was synthesized using general procedure 3 and 3-hydroxypiperidine (18b). The crude reaction was purified using preparative HPLC to afford the desired compound as a transparent oil (25 mg, 26% yield) with a purity of 99% by UHPLC-MS. UHPLC-MS (ESI+APCI) m/z calcd. for C13H15NO3 [M+H]⁺=234. Found: 234. Retention time: 0.85 min. ¹H NMR (300 MHz, CDCl3) δ 10.01 (s, 1H), 7.89 (d, J=7.71 Hz, 2H), 7.54 (d, J=6.19 Hz, 2H), 3.90 (m, 1H), 3.62 (m, 2H), 3.26 (m, 2H), 1.89 (m, 2H), 1.63 (m, 1H), 1.40 (m, 1H) ppm. ¹³C NMR (75 MHz, CDCl3) δ 191.5, 169.9, 141.7, 136.8, 129.9, 127.7, 127.5, 66.1, 65.8, 53.8, 49.0, 48.0, 42.5, 32.6, 31.9, 22.8, 21.4, 18.5 and 17.2 ppm.

4-(morpholine-4-carbonyl)benzaldehyde (19c). Compound 19c was synthesized using general procedure 3 and morpholine (18c). The crude reaction was purified using preparative HPLC to afford the desired compound as a white amorphous solid (48 mg, 55% yield) with a purity 99% by UHPLC-MS. UHPLC-MS (ESI+APCI) m/z calcd. for _{C12H13NO3} [M+H]⁺=220. Found: 220. Retention time: 0.86 min. ¹H NMR (300 MHz, CDCl3) δ 10.02 (s, 1H), 7.91 (d, J=8.11 Hz, 2H), 7.54 (d, J=8.14 Hz, 2H), 3.77 (s, 4H), 3.61 (s, 2H), 3.38 (s, 2H) ppm. ¹³C NMR (75 MHz, CDCl3) δ 191.3, 169.0, 140.9, 137.0, 129.9, 127.6 and 66.7 ppm.

4-(4-methoxypiperidine-1-carbonyl)benzaldehyde (19d). Compound 19d was synthesized using general procedure 3 and 4-methoxypiperidine (18d). The crude reaction was purified using preparative HPLC to afford the desired compound as a yellow oil (35 mg, 35% yield) with a purity of 99% by UHPLC-MS. UHPLC-MS (ESI+APCI) m/z calcd. for C₁₄H₁₇NO₃ [M+H]⁺=248. Found: 248. Retention time: 1.01 min. ¹H NMR (300 MHz, CDCl3) δ 10.02 (s, 1H), 7.91 (d, J=8.22 Hz, 2H), 7.52 (d, J=8.11 Hz, 2H), 3.97 (bp, 1H), 3.51 (m, 3H), 3.34 (s, 3H), 3.17 (bp, 1H), 1.92 (bp, 1H), 1.73 (bp, 2H), 1.55 (bp, 1H) ppm. ¹³C NMR (75 MHz, CDCl3) δ 191.2, 168.7, 141.7, 136.6, 129.7, 127.2, 74.7, 55.6, 44.4, 38.9, 31.0 and 29.9 ppm.

1-(4-formylbenzoyl)piperidine-4-carbonitrile (19e). Compound 19e was synthesized using general procedure 3 and 4-cyanopiperidine (18e). The crude reaction was purified using preparative HPLC to afford the desired compound as a transparent oil (26 mg, 27% yield) with a purity of 99% by UHPLC-MS. UHPLC-MS (ESI+APCI) m/z calcd. for C₁₄H₁₄N202 [M+H]⁺=243. Found: 243. Retention time: 0.97 min. ¹H NMR (300 MHz, CDCl3) δ 10.04 (s, 1H), 7.94 (d, J=8.29 Hz, 2H), 7.54 (d, J=8.10 Hz, 2H), 3.85 (bd, 2H), 3.47 (bd, 2H), 2.95 (m, 1H), 1.92 (bp, 4H) ppm. ¹³C NMR (75 MHz, CDCl3) δ 191.0, 168.8, 161.8, 140.6, 136.8, 129.7, 127.1, 126.9, 120.1, 45.2, 45.1, 39.8, 39.7, 28.7, 28.6, 28.0, 27.9 and 26.0 ppm.

4-(4-formylbenzoyl)-N,N-dimethylpiperazine-1-carboxamide (19f). Compound 19f was synthesized using general procedure 3 and N,N-dimethylpiperazine-1-carboxamide (18f). The crude reaction was purified using preparative HPLC to afford the desired compound as a yellow oil (51 mg, 44% yield) with a purity of 98% by UHPLC-MS. UHPLC-MS (ESI+APCI) m/z calcd. for C15H₁₉N₃O₃ [M+H]⁺=290. Found: 290. Retention time: 0.94 min. ¹H NMR (300 MHz, CDCl3) δ 10.02 (s, 1H), 7.91 (d, J=8.22 Hz, 2H), 7.52 (d, J=8.10 Hz, 2H), 3.77 (bp, 2H), 3.37 (bp, 2H), 3.29 (bp, 2H), 3.16 (bp, 2H), 2.83 (s, 6H) ppm. ¹³C NMR (75 MHz, CDCl3) δ 191.1, 168.9, 164.1, 140.9, 136.6, 129.7, 127.3, 53.2, 46.6, 41.7 and 38.1 ppm.

4-[4-(2-hydroxyethyl)piperidine-1-carbonyl]benzaldehyde (19g). Compound 19g was synthesized using general procedure 3 and 4-piperidineethanol (18g). The crude reaction was purified using preparative HPLC to afford the desired compound as a yellow oil (39 mg, 37% yield) with a purity of 98% by UHPLC-MS. UHPLC-MS (ESI+APCI) m/z calcd. for C15H₁₉NO3 [M+H]⁺=262. Found: 262. Retention time: 0.95 min. ¹H NMR (300 MHz, CDCl3) δ 9.99 (s, 1H), 7.87 (d, J=8.22 Hz, 2H), 7.49 (d, J=8.10 Hz, 2H), 4.64 (d, J=12.42 Hz, 1H), 3.64 (t, 2H), 3.56 (d, J=10.16 Hz, 1H), 2.97 (t, 1H), 2.74 (t, 1H), 2.02 (s, 1H). 1.72 (m, 3H), 1.50 (q, 2H), 1.15 (m, 2H) ppm. ¹³C NMR (75 MHz, CDCl3) δ 191.5, 168.8, 142.0, 136.7, 129.8, 127.3, 59.9, 47.9, 42.4, 38.8, 32.8, 32.6 and 31.8 ppm.

4-formyl-N-[(oxan-4-yl)methyl]benzamide (19h). Compound 19h was synthesized using general procedure 3 and 4-aminomethyltetrahydropyran (18h). The crude reaction was purified using preparative HPLC to afford the desired compound as a yellow amorphous solid (42 mg, 42% yield) with a purity of 99% by UHPLC-MS. UHPLC-MS (ESI+APCI) m/z calcd. for C₁₄H₁₇NO₃ [M+H]⁺=248. Found: 248. Retention time: 0.96 min. ¹H NMR (300 MHz, CDCl3) δ 10.04 (s, 1H), 7.90 (s, 4H), 6.63 (s, 1H), 3.96 (dd, J=11.39 Hz, J=3.63 Hz, 2H), 3.35 (m, 4H), 1.88 (m, 1H), 1.65 (dd, J=12.91 Hz, J=1.69 Hz, 2H), 1.36 (dq, J=12.08 Hz, J=4.46 Hz, 2H) ppm. ¹³C NMR (75 MHz, CDCl3) δ 191.1, 166.2, 139.3, 137.7, 129.4, 127.1, 67.0, 45.4, 34.8 and 30.2 ppm.

4-formyl-N-[(3-methoxyphenyl)methyl]benzamide (19i). Compound 19i was synthesized using general procedure 3 and 3-methoxybenzylamine (18i). The crude reaction was purified using preparative HPLC to afford the desired compound as a brown amorphous solid (46 mg, 43% yield) with a purity of 80% by UHPLC-MS. UHPLC-MS (ESI+APCI) m/z calcd. for C₁₆H₁₅NO₃ [M+H]⁺=270. Found: 270. Retention time: 1.24 min. ¹H NMR (300 MHz, CDCl3) δ 10.01 (s, 1H), 7.89 (s, 4H), 7.22 (d, J=7.61 Hz, 1H), 6.89 (d, J=7.57 Hz, 1H), 6.84 (t, 1H), 6.80 (dd, J=8.21 Hz, J=2.03 Hz, 1H), 6.69 (bp, 1H), 4.58 (d, J=5.66 Hz, 2H), 3.75 (s, 3H) ppm. ¹³C NMR (75 MHz, CDCl3) δ 191.5, 166.2, 159.9, 139.5, 139.3, 138.7, 129.9, 129.8, 129.5, 127.9, 127.7, 120.1, 113.6, 113.0, 55.2 and 44.2 ppm.

4-formyl-N-[(4-methoxyphenyl)methyl]benzamide (19j). Compound 19j was synthesized using general procedure 3 and 4-methoxybenzylamine (18j). The crude reaction was purified using preparative HPLC to afford the desired compound as a yellow amorphous solid (53 mg, 60% yield) with a purity of 99% by UHPLC-MS. UHPLC-MS (ESI+APCI) m/z calcd. for C₁₆H₁₅NO₃ [M+H]⁺=270. Found: 270. Retention time: 1.20 min. ¹H NMR (300 MHz, CDCl3) δ 10.05 (s, 1H), 7.92 (s, 4H), 7.28 (d, J=8.66 Hz, 2H), 6.88 (d, J=8.66 Hz, 2H), 6.56 (bp, 1H), 4.57 (d, J=5.53 Hz, 2H), 3.80 (s, 3H) ppm. ¹³C NMR (75 MHz, CDCl3) δ 191.5, 166.2, 159.3, 139.6, 138.2, 129.8, 129.7, 129.4, 127.7, 114.3, 55.3 and 43.9 ppm.

4-formyl-N-(1-methyl-1H-pyrazol-3-yl)benzamide (19k). Compound 19k was synthesized using general procedure 3 and 1-methyl-1H-pyrazol-3-ylamine (18k). The crude reaction was purified using preparative HPLC to afford the desired compound as a yellow solid (16 mg, 22% yield) with a purity of 99% by UHPLC-MS. UHPLC-MS (ESI+APCI) m/z calcd. for C₁₂H₁₁N₃O₂ [M+H]⁺=230. Found: 230. Retention time: 0.93 min. ¹H NMR (300 MHz, CDCl3) δ 10.08 (s, 1H), 9.50 (bp, 1H), 8.04 (d, J=8.26 Hz, 2H), 7.96 (d, J=8.37 Hz, 2H), 7.30 (d, J=2.25 Hz, 1H), 6.86 (d, J=2.19 Hz, 1H), 3.69 (s, 3H) ppm. ¹³C NMR (75 MHz, CDCl3) δ 191.3, 163.5, 146.7, 139.1, 138.2, 131.0, 129.7, 127.8, 97.6 and 38.4 ppm.

4-formyl-N-[(1-methyl-1H-pyrazol-4-yl)methyl]benzamide (191). Compound 191 was synthesized using general procedure 3 and (1-methyl-1H-pyrazol-4-yl)methanamine (181). The crude reaction was purified using preparative HPLC to afford the desired compound as a yellow solid (21 mg, 21% yield) with a purity of 90% by UHPLC-MS. UHPLC-MS (ESI+APCI) m/z calcd. for C₁₃H₁₃N302 [M+H]⁺=244. Found: 244. Retention time: 0.88 min. ¹H NMR (300 MHz, CDCl3) δ 10.03 (s, 1H), 7.90 (s, 4H), 7.45 (s, 1H), 7.40 (s, 1H), 6.57 (bp, 1H), 4.47 (d, J=5.45 Hz, 2H), 3.85 (s, 3H) ppm. ¹³C NMR (75 MHz, CDCl3) δ 191.5, 166.1, 139.4, 138.7, 138.2, 129.8, 129.6, 127.6, 118.0, 38.9 and 34.6 ppm.

4-formyl-N-(6-methoxy-4-methylpyridin-3-yl)benzamide (19m). Compound 19m was synthesized using general procedure 3 and 5-amino-2-methoxy-4-methylpyridine (18m). The crude reaction was purified using preparative HPLC to afford the desired compound as a yellow solid (46 mg, 43% yield) with a purity of 97% by UHPLC-MS. UHPLC-MS (ESI+APCI) m/z calcd. for CI₅H₁₄N₂O₃ [M+H]⁺=271. Found: 271. Retention time: 1.10 min. ¹H NMR (300 MHz, CDCl3) δ 10.09 (s, 1H), 8.24 (s, 1H), 8.01 (q, 4H), 7.77 (s, 1H), 6.64 (s, 1H), 3.92 (s, 3H), 2.25 (s, 3H) ppm. ¹³C NMR (75 MHz, CDCl3) δ 191.2, 165.2, 162.6, 145.4, 143.5, 138.9, 138.3, 129.8, 127.7, 126.1, 111.5, 53.4 and 17.6 ppm.

2-formylbenzene-1-sulfonyl-chloride (21). Synthesis of 21 was performed as per reported procedure by Fish et al. To sodium 2-formylbenzene-1-sulfonate (20) (9.51 mmol, 1 Eq.) was added thionyl chloride (104.6 mmol, 11 Eq) at room temperature. After stirring for 10 min, catalytic DMF (0.15 mL) was added to the reaction mixture. The reaction mixture was then stirred at room temperature for an additional 2 minutes, before being heated to reflux for 3 min. After complete consumption of the starting materials—monitored by TLC (DCM/MeOH 1:1)—the mixture was then poured in ice water (vapours formation) and extracted with diethyl ether (3×50 ml). The organic layer was then washed with brine (2×30 ml), dried over Magnesium Sulfate and evaporated under reduced pressure to afford the desired product as a white amorphous solid (950 mg, 50% yield) with a purity of 90% by ¹H NMR. ¹H NMR (300 MHz, CDCl3): 7.86 (d, J=7.72 Hz, 1H), 7.80 (dd, J=7.56 Hz, J=1.06 Hz, 1H), 7.74 (d, J=7.52 Hz, 1H), 7.62 (d, J=7.74 Hz, 1H), 7.22 (s, 1H) ppm. Since the proton spectrum was conform to the reported literature, the compound was used as is.

General procedure 4 (23a-aa, 24a-1). To a solution of the corresponding amine (0.29 mmol, 1 Eq.) in DCM (1 ml) was added Triethylamine (0.88 mmol, 3 Eq.). After stirring at room temperature for 10 min., a solution of formylbenzenesulfonyl chloride (21) (0.29 mmol, 1 Eq.) in DCM (1 ml) was added. The reaction mixtures was then stirred at room temperature overnight. After complete consumption of the starting materials—monitored by TLC (DCM/MeOH 9:1) and UHPLC-MS—The reaction mixture was diluted with a saturated solution NaHCO₃ (1 ml). The organic layer was separated, dried over magnesium sulfate, and concentrated under reduced pressure. Compound was purified by preparative HPLC.

During work up of compounds 23n, q, r, s, u, y and z a precipitate formed after addition of a saturated solution NaHCO₃. The resulting precipitate was filtered, and the precipitate was washed with a saturated solution NaHCO₃ and water. The resulting precipitate was dried under reduced pressure to afford the desired compounds.

4-[(2-chloro-5-methoxy-1H-1,3-benzodiazol-1-yl)sulfonyl]benzaldehyde/4-[(2-chloro-6-methoxy-1H-1,3-benzodiazol-1-yl)sulfonyl]benzaldehyde (mixture 1:1) (23a). Compound 23a was synthesized using general procedure 4 and 2-chloro-5-methoxy-1H-1,3-benzodiazole (22a). After purification the desired compound was afforded as a white amorphous solid (34 mg, 35% yield) with a purity of 92% by UHPLC-MS. UHPLC-MS (ESI+APCI) m/z calcd. for C₁₅H₁₁ClN₂O₄S [M+H]⁺=351. Found: 351. Retention time: 1.54 min.

¹H NMR (300 MHz, CDCl3) evidenced the 1:1 mixture. δ 10.08 (s, 1H), 10.07 (s, 1H), 8.17 (d, J=8.36 Hz, 2H), 8.15 (d, J=8.28 Hz, 2H), 8.03 (d, J=8.54 Hz, 2H), 8.02 (d, J=8.52 Hz, 2H), 7.97 (d, J=9.09 Hz, 1H), 7.62 (d, J=2.28 Hz, 1H), 7.51 (d, J=8.85 Hz, 1H), 7.01 (d, J=2.12 Hz, 1H), 7.03 (dd, J=2.41 Hz, J=15.29 Hz, 1H), 7.00 (dd, J=2.42 Hz, J=15.04 Hz, 1H), 3.92 (s, 3H), 3.83 (s, 3H) ppm. ¹³C NMR (75 MHz, CDCl3) δ 190.0, 158.6, 158.1, 142.2, 142.0, 140.3, 140.3, 130.4, 128.1, 128.1, 120.6, 115.0, 114.3, 113.9, 102.8, 98.3, 56.0, 55.7 ppm.

4-formyl-N-(1-methyl-1H-pyrazol-3-yl)benzene-1-sulfonamide (23b). Compound 23b was synthesized using general procedure 4 and 1-methyl-1H-pyrazol-3-ylamine (22b). After purification the desired compound was afforded as a white amorphous solid (13 mg, 20% yield) with a purity of 99% by UHPLC-MS. UHPLC-MS (ESI+APCI) m/z calcd. for C₁₁H₁₁N₃O₃S [M+H]⁺=266. Found: 266. Retention time: 0.99 min. ¹H NMR (300 MHz, CDCl3) δ 10.06 (s, 1H), 7.86 (dd, J=8.64 Hz, J=14.81 Hz, 2H), 7.23 (d, J=2.33 Hz, 0.5H), 6.27 (d, J=2.36 Hz, 0.5H), 3.84 (s, 1.6H) ppm. ¹³C NMR (75 MHz, CDCl3) δ 190.8, 145.7, 144.6, 138.9, 132.0, 129.9, 127.6, 97.8, 38.9 ppm.

4-formyl-N,N-dimethylbenzene-1-sulfonamide (23c). Compound 23c was synthesized using general procedure 4 and dimethylamine (22c) (2M solution in THF). After purification the desired compound was afforded as a white amorphous solid (13 mg, 24% yield) with a purity of 99% by UHPLC-MS. UHPLC-MS (ESI+APCI) m/z calcd. for C₉H₁₁NO₃S [M+H]⁺=214. Found: 214. Retention time: 1.07 min. ¹H NMR (300 MHz, CDCl3) δ 10.12 (s, 1H), 8.05 (d, J=8.45 Hz, 2H), 7.94 (d, J=8.33 Hz, 2H), 2.75 (s, 6H) ppm. ¹³C NMR (75 MHz, CDCl3) δ 190.8, 141.0, 138.9, 130.1, 128.3, 37.8 ppm.

4-[(3-methoxyazetidin-1-yl)sulfonyl]benzaldehyde (23d). Compound 23d was synthesized using general procedure 4 and 3-azetidinyl methyl ether hydrochloride (22d). After purification the desired compound was afforded as a white amorphous solid (17 mg, 27% yield) with a purity of 99% by UHPLC-MS. UHPLC-MS (ESI+APCI) m/z calcd. for C₁₁H₁₃NO₄S [M+H]⁺=256. Found: 256. Retention time: 1.08 min. ¹H NMR (300 MHz, CDCl3) δ 10.13 (s, 1H), 8.08 (d, J=8.52 Hz, 2H), 8.00 (d, J=8.33 Hz, 2H), 4.06 (m, 3H), 3.61 (m, 2H), 3.15 (s, 3H) ppm. ¹³C NMR (75 MHz, CDCl3) δ 190.8, 140.1, 139.2, 130.1, 128.8, 67.7, 58.1, 56.2 ppm.

4-{[(2S)-2-methylpyrrolidin-1-yl]sulfonyl}benzaldehyde (23e). Compound 23e was synthesized using general procedure 4 and (2S)-2-methylpyrrolidine (22e). After purification the desired compound was afforded as a brown amorphous solid (39 mg, 52% yield) with a purity of 99% by UHPLC-MS. UHPLC-MS (ESI+APCI) m/z calcd. for C₁₂H₁₅NO₃S [M+H]⁺=254. Found: 254. Retention time: 1.31 min. 1H NMR (300 MHz, CDCl3) δ 10.09 (s, 1H), 8.03 (d, J=8.67 Hz, 2H), 7.98 (d, J=8.64 Hz, 2H), 3.74 (m, 1H), 3.48 (m, 1H), 3.18 (m, 1H), 1.86 (m, 1H), 1.71 (m, 1H), 1.54 (m, 1H), 1.30 (d, J=6.36 Hz, 3H) ppm. ¹³C NMR (75 MHz, CDCl3) δ 190.7, 143.0, 138.4, 129.9, 127.7, 56.2, 48.8, 32.2, 23.7, 22.4 ppm.

2-(piperidine-1-sulfonyl)benzaldehyde (23f). Compound 23f was synthesized using general procedure 4 and piperidine (22f). After purification the desired compound was afforded as a transparent oil (7 mg, 10% yield) with a purity of 99% by UHPLC-MS. UHPLC-MS (ESI+APCI) m/z calcd. for C₁₂H₁₅NO₃S [M+H]⁺=254. Found: 254. Retention time: 1.35 min. ¹H NMR (300 MHz, CDCl3) δ 10.11 (s, 1H), 8.03 (d, J=8.50 Hz, 2H), 7.91 (d, J=8.27 Hz, 2H), 3.03 (t, 4H), 1.64 (m, 4H), 1.43 (m, 2H) ppm. ¹³C NMR (75 MHz, CDCl3) δ 190.7, 147.7, 138.6, 129.8, 128.0, 46.7, 24.9, 23.2 ppm.

4-[(4-methyl-1,4-diazepan-1-yl)sulfonyl]benzaldehyde (23g)—formate salt. Compound 23g was synthesized using general procedure 4 and 1-methyl-1,4-diazepane (22g). After purification the desired compound was afforded as an off-yellow amorphous solid (40.3 mg, 51% yield) with a purity of 99% by UHPLC-MS. UHPLC-MS (ESI+APCI) m/z calcd. for C₁₃H₁₈N₂O₃S [M+H]⁺=283. Found: 283. Retention time: 0.58 min. ¹H NMR (300 MHz, CDCl3) δ 10.08 (s, 1H), 9.01 (bp, 1H, formate proton), 8.33 (s, 0.5H, formate proton), 8.02 (d, J=8.47 Hz, 2H), 7.92 (d, J=8.32 Hz, 2H), 3.50 (m, 2H), 3.39 (t, 2H), 2.86 (q, 4H), 2.48 (s, 3H), 2.01 (m, 2H) ppm. ¹³C NMR (75 MHz, CDCl3) δ 190.5, 167.3 (formate carbon), 143.6, 138.6, 130.1, 127.4, 58.3, 55.5, 46.5, 45.9, 45.3, 25.7 ppm.

4-[(4-methylpiperazin-1-yl)sulfonyl]benzaldehyde (23h). Compound 23h was synthesized using general procedure 4 and 1-methylpiperazine (22h). After purification the desired compound was afforded as a white amorphous solid (47.6 mg, 60% yield) with a purity of 99% by UHPLC-MS. UHPLC-MS (ESI+APCI) m/z calcd. for C₁₂H₁₆N₂O₃S [M+H]⁺=269. Found: 269. Retention time: 0.55 min. ¹H NMR (300 MHz, CDCl3) δ 10.10 (s, 1H), 9.95 (bp, 1H, formate proton), 8.07 (s, 1H, formate proton), 8.04 (d, J=8.47 Hz, 2H), 7.90 (d, J=8.30 Hz, 2H), 3.22 (m, 3H), 2.80 (t, 3H), 2.43 (s, 2.5H) ppm. ¹³C NMR (75 MHz, CDCl3) δ 190.5, 165.3 (formate carbon), 140.5, 139.9, 130.0, 128.0, 52.9, 44.3, 44.1 ppm.

4-[(4-ethylpiperazin-1-yl)sulfonyl]benzaldehyde (23i)—formate salt. Compound 23i was synthesized using general procedure 4 and 1-ethylpiperazine (22i). After purification the desired compound was afforded as an off-yellow solid (37 mg, 53% yield) with a purity of 99% by UHPLC-MS. UHPLC-MS (ESI+APCI) m/z calcd. for C₁₃H₁₈N₂O₃S [M+H]⁺=283. Found: 283. Retention time: 0.56 min. ¹H NMR (300 MHz, CDCl3) δ 10.09 (s, 1H), 8.08 (s, 0.6H, formate proton), 8.03 (d, J=8.45 Hz, 2H), 7.89 (d, J=8.30 Hz, 2H), 7.80 (bp, 1H, formate proton), 3.20 (m, 4H), 2.76 (m, 4H), 2.60 (q, 2H), 1.02 (t, 3H) ppm. ¹³C NMR (75 MHz, CDCl3) δ 190.7, 165.8 (formate carbon), 140.6, 139.1, 130.2, 128.3, 127.6, 51.6, 51.0, 44.8, 10.5 ppm.

4-{[4-(propan-2-yl)piperazin-1-yl]sulfonyl}benzaldehyde (23j)—formate salt. Compound 23j was synthesized using general procedure 4 and 1-isopropylpiperazine (22j). After purification the desired compound was afforded as a white amorphous solid (14 mg, 16% yield) with a purity of 99% by UHPLC-MS. UHPLC-MS (ESI+APCI) m/z calcd. for C₁₄H₂₀N₂O₃S [M+H]⁺=297. Found: 297. Retention time: 0.64 min. ¹H NMR (300 MHz, CDCl3) δ 10.11 (s, 1H), 8.11 (s, 0.5H, formate proton) 8.04 (d, J=8.38 Hz, 2H), 7.91 (d, J=8.31 Hz, 2H), 6.50 (bp, 1H, formate proton), 3.21 (t, 4H), 2.93 (m, 1H), 2.79 (t, 4H), 1.09 (d, J=6.60 Hz, 6H) ppm. ¹³C NMR (75 MHz, CDCl3) δ 190.5, 165.4 (formate carbon), 140.6, 138.8, 130.0, 128.2, 54.9, 47.2, 45.1, 17.5 ppm.

4-{[4-(2-methylpropyl)piperazin-1-yl]sulfonyl}benzaldehyde (23k). Compound 23k was synthesized using general procedure 4 and 1-isobutylpiperazine (22k). After purification the desired compound was afforded as a yellow amorphous solid (50 mg, 55% yield) with a purity of 99% by UHPLC-MS. UHPLC-MS (ESI+APCI) m/z calcd. for C15H22N₂O₃S [M+H]⁺=311. Found: 311. Retention time: 0.71 min. 1H NMR (300 MHz, CDCl3) δ 10.11 (s, 1H), 8.04 (d, J=8.42 Hz, 2H), 7.91 (d, J=8.30 Hz, 2H), 3.08 (m, 4H), 2.51 (t, 4H), 2.11 (d, J=7.36 Hz, 2H), 1.72 (m, 1H), 0.82 (d, J=6.58 Hz, 6H) ppm. ¹³C NMR (75 MHz, CDCl3) δ 190.6, 140.8, 138.7, 129.9, 128.2, 65.9, 52.2, 45.6, 25.0, 21.0 ppm.

4-{[4-(2-methoxyethyl)piperazin-1-yl]sulfonyl}benzaldehyde (231). Compound 231 was synthesized using general procedure 4 and 1-(2-methoxyethyl)piperazine (221). After purification the desired compound was afforded as a yellow oil (38 mg, 42% yield) with a purity of 99% by UHPLC-MS. UHPLC-MS (ESI+APCI) m/z calcd. for C₁₄H₂₀N₂O₄S [M+H]⁺=313. Found: 313. Retention time: 0.64 min. ¹H NMR (300 MHz, CDCl₃) δ 10.09 (s, 1H), 8.02 (d, J=8.49 Hz, 2H), 7.89 (d, J=8.27 Hz, 2H), 3.44 (t, 2H), 3.27 (s, 3H), 3.10 (t, 4H), 2.60 (m, 6H) ppm. ¹³C NMR (75 MHz, CDCl₃) δ 190.6, 140.5, 138.8, 129.9, 128.2, 69.3, 58.7, 57.1, 52.1, 45.5 ppm.

4-{[4-(pyrrolidin-1-yl)piperidin-1-yl]sulfonyl}benzaldehyde (23m). Compound 23m was synthesized using general procedure 4 and 4-(1-pyrrolidinyl)piperidine (22m). After purification the desired compound was afforded as a yellow amorphous soiled (40 mg, 42% yield) with a purity of 99% by UHPLC-MS. UHPLC-MS (ESI+APCI) m/z calcd. for C16H22N₂O₃S [M+H]⁺=323. Found: 323. Retention time: 0.69 min. ¹H NMR (300 MHz, CDCl3) δ 10.09 (s, 1H), 8.02 (d, J=8.41 Hz, 2H), 7.91 (d, J=8.32 Hz, 2H), 3.70 (d, J=12.26 Hz, 2H), 2.49 (m, 6H), 1.94 (m, 3H), 1.73 (m, 4H), 1.61 (d, J=11.03 Hz, 2H) ppm. ¹³C NMR (75 MHz, CDCl3) δ 190.7, 141.6, 138.6, 129.9, 128.0, 60.2, 51.1, 44.6, 30.3, 23.0 ppm.

4-{[4-(dimethylamino)piperidin-1-yl]sulfonyl}benzaldehyde (23n)—formate salt. Compound 23n was synthesized using general procedure 4 and 4-(dimethylamino)piperidine (22n). After purification the desired compound was afforded as a white amorphous soiled (16 mg, 18% yield) with a purity of 99% by UHPLC-MS. UHPLC-MS (ESI+APCI) m/z calcd. for C₁₄H2₀N₂O₃S [M+H]⁺=297. Found: 297. Retention time: 0.65 min. ¹H NMR (300 MHz, CDCl₃) δ 10.11 (s, 1H), 9.32 (bp, 1H, formate proton), 8.32 (s, 1H, formate proton), 8.04 (d, J=8.48 Hz, 2H), 7.91 (d, J=8.26 Hz, 2H), 3.94 (d, J=12.12 Hz, 2H), 2.75 (m, 1H), 2.47 (s, 6H), 2.38 (td, J=2.13 Hz, J=12.09 Hz, 2H), 2.00 (m, 2H), 1.71 (qd, J=4.04 Hz, J=12.21 Hz, 2H) ppm. ¹³C NMR (75 MHz, CDCl₃) δ 190.4, 167.0 (formate carbon), 141.1, 138.9, 130.0, 128.0, 60.5, 45.0, 39.2, 25.7 ppm.

4-[(4-acetylpiperazin-1-yl)sulfonyl]benzaldehyde (23o). Compound 23o was synthesized using general procedure 4 and 1-acetylpiperazine (220). After purification the desired compound was afforded as a white amorphous soiled (30 mg, 34% yield) with a purity of 99% by UHPLC-MS. UHPLC-MS (ESI+APCI) m/z calcd. for C13H16N₂O₄S [M+H]⁺=297. Found: 297. Retention time: 1.01 min. ¹H NMR (300 MHz, CDCl3) δ 10.11 (s, 1H), 8.05 (d, J=8.44 Hz, 2H), 7.90 (d, J=8.30 Hz, 2H), 3.70 (t, 2H), 3.55 (t, 2H), 3.04 (m, 4H), 2.02 (s, 3H) ppm. ¹³C NMR (75 MHz, CDCl3) δ 190.4, 166.6, 140.5, 139.0, 130.1, 128.11, 45.9, 45.6, 45.5, 40.5, 21.0 ppm.

4-[(3-oxopiperazin-1-yl)sulfonyl]benzaldehyde (23p). Compound 23p was synthesized using general procedure 4 and piperazin-2-one (22p). After purification the desired compound was afforded as a white amorphous soiled (34 mg, 43% yield) with a purity of 99% by UHPLC-MS. UHPLC-MS (ESI+APCI) m/z calcd. for C11H12N2O₄S [M+H]⁺=269. Found: 269. Retention time: 0.91 min. ¹H NMR (300 MHz, DMSO-d6) δ 10.14 (s, 1H), 8.15 (d, J=8.40 Hz, 2H), 8.06 (bp, 1H), 8.03 (d, J=8.27 Hz, 2H), 3.57 (s, 2H), 3.26 (m, 2H), 3.18 (m, 2H) ppm. ¹³C NMR (100 MHz, DMSO-d6) δ 193.1, 130.9, 128.7, 48.5, 42.8, 32.0, 31.7, 31.6, 30.8 ppm.

4-[(3-hydroxypiperidin-1-yl)sulfonyl]benzaldehyde (23q). Compound 23q was synthesized using general procedure 4 and 3-hydroxypiperidine (22q). After purification the desired compound was afforded as a white amorphous soiled (38.5 mg, 49% yield) with a purity of 99% by UHPLC-MS. UHPLC-MS (ESI+APCI) m/z calcd. for _{C12H15NO4S} [M+H]+=270. Found: 270. Retention time: 1.02 min. ¹H NMR (300 MHz, CDCl3) δ 10.11 (s, 1H), 8.04 (d, J=8.46 Hz, 2H), 7.93 (d, J=8.32 Hz, 2H), 3.87 (m, 1H), 3.40 (dd, J=3.53 Hz, J=11.43 Hz, 1H), 3.20 (m, 1H), 2.82 (m, 1H), 2.75 (dd, J=7.40 Hz, J=11.42 Hz, 1H), 2.11 (bp, 1H), 1.81 (m, 2H), 1.63 (m, 1H), 1.42 (m, 1H) ppm. ¹³C NMR (75 MHz, CDCl3) δ 190.6, 141.5, 138.7, 130.0, 128.0, 65.4, 52.2, 46.0, 31.4, 21.6 ppm.

4-[(3-methoxypiperidin-1-yl)sulfonyl]benzaldehyde (23r). Compound 23r was synthesized using general procedure 4 and 3-methoxypiperidine (22r). After purification the desired compound was afforded as a white amorphous soiled (27 mg, 32% yield) with a purity of 99% by UHPLC-MS. UHPLC-MS (ESI+APCI) m/z calcd. for C₁₃H₁₇NO₄S [M+H]⁺=284. Found: 284. Retention time: 1.21 min. ¹H NMR (300 MHz, CDCl3) δ 10.10 (s, 1H), 8.03 (d, J=8.47 Hz, 2H), 7.93 (d, J=8.29 Hz, 2H), 3.60 (dd, J=3.79 Hz, J=11.41 Hz, 1H), 3.41 (m, 1H), 3.35 (s, 3H), 3.31 (dd, J=4.24 Hz, J=8.36 Hz, 1H), 2.55 (m, 2H), 1.85 (m, 2H), 1.60 (m, 1H), 1.28 (m, 1H) ppm. ¹³C NMR (75 MHz, CDCl₃) δ 190.6, 141.7, 138.6, 129.9, 127.9, 74.3, 56.2, 49.0, 46.0, 29.0, 22.0 ppm.

4-[(4-hydroxypiperidin-1-yl)sulfonyl]benzaldehyde (23s). Compound 23s was synthesized using general procedure 4 and 4-hydroxypiperidine (22s). After purification the desired compound was afforded as a white amorphous soiled (19 mg, 20% yield) with a purity of 99% by UHPLC-MS. UHPLC-MS (ESI+APCI) m/z calcd. for C₁₂H₁₅NO₄S [M+H]⁺=270. Found: 270. Retention time: 0.99 min. ¹H NMR (300 MHz, CDCl3) δ 10.10 (s, 1H), 8.03 (d, J=8.48 Hz, 2H), 7.92 (d, J=8.30 Hz, 2H), 3.80 (m, 1H), 3.31 (m, 2H), 2.96 (m, 2H), 1.92 (m, 2H), 1.67 (m, 2H), 1.57 (bs, 1H) ppm. ¹³C NMR (75 MHz, CDCl3) δ 190.8, 141.7, 138.9, 130.1, 128.2, 65.4, 42.9, 33.1 ppm.

4-[(4-methoxypiperidin-1-yl)sulfonyl]benzaldehyde (23t). Compound 23t was synthesized using general procedure 4 and 4-methoxypiperidine (22t). After purification the desired compound was afforded as a white amorphous soiled (42 mg, 51% yield) with a purity of 99% by UHPLC-MS. UHPLC-MS (ESI+APCI) m/z calcd. for C₁₃H₁₇NO₄S [M+H]⁺=284. Found: 284. Retention time: 1.22 min. ¹H NMR (300 MHz, CDCl3) δ 10.10 (s, 1H), 8.03 (d, J=8.45 Hz, 2H), 7.90 (d, J=8.30 Hz, 2H), 3.29 (m, 1H), 3.23 (s, 3H), 3.08 (m, 4H), 1.85 (m, 2H), 1.72 (m, 2H) ppm. ¹³C NMR (75 MHz, CDCl3) δ 190.7, 141.6, 138.7, 129.9, 128.0, 73.2, 55.6, 42.6, 29.4 ppm.

4-(morpholine-4-sulfonyl)benzaldehyde (23u). Compound 23u was synthesized using general procedure 4 and morpholine (22u). After purification the desired compound was afforded as a white amorphous soiled (14 mg, 23% yield) with a purity of 99% by UHPLC-MS. UHPLC-MS (ESI+APCI) m/z calcd. for C₁₁H₁₃NO₄S [M+H]⁺=256. Found: 256. Retention time: 1.07 min. ¹H NMR (300 MHz, CDCl3) δ 10.12 (s, 1H), 8.06 (d, J=8.48 Hz, 2H), 7.92 (d, J=8.30 Hz, 2H), 3.75 (m, 4H), 3.04 (m, 4H) ppm. ¹³C NMR (75 MHz, CDCl3) δ 190.7, 140.6, 139.1, 130.2, 128.4, 66.0, 45.9 ppm.

4-[(2,6-dimethylmorpholin-4-yl)sulfonyl]benzaldehyde (23v). Compound 23v was synthesized using general procedure 4 and 2,6-dimethylmorpholine (22v). After purification the desired compound was afforded as a white amorphous soiled (27 mg, 32% yield) with a purity of 99% by UHPLC-MS. UHPLC-MS (ESI+APCI) m/z calcd. for C₁₃H₁₇NO₄S [M+H]⁺=284. Found: 284. Retention time: 1.29 min. ¹H NMR (300 MHz, CDCl3) δ 10.12 (s, 1H), 8.06 (d, J=8.47 Hz, 2H), 7.91 (d, J=8.26 Hz, 2H), 3.70 (m, 2H), 3.60 (d, J=10.14 Hz, 2H), 1.97 (m, 2H), 1.13 (d, J=6.27 Hz, 6H) ppm. ¹³C NMR (75 MHz, CDCl3) δ 190.5, 140.6, 138.8, 130.0, 128.1, 71.1, 50.5, 18.4 ppm.

N-cyclohexyl-4-formyl-N-methylbenzene-1-sulfonamide (23w). Compound 23w was synthesized using general procedure 4 and N-methylcyclohexylamine (22w). After purification the desired compound was afforded as a white amorphous soiled (42 mg (51% yield) with a purity of 99% by UHPLC-MS. UHPLC-MS (ESI+APCI) m/z calcd. for CI₄H₁₉NO₃S [M+H]⁺=282. Found: 200. Retention time: 1.53 min. ¹H NMR (300 MHz, CDCl3) δ 10.08 (s, 1H), 8.00 (d, J=8.55 Hz, 2H), 7.95 (d, J=8.51 Hz, 2H), 3.77 (m, 1H), 2.77 (s, 3H), 1.73 (m, 2H), 1.59 (d, J=11.55 Hz, 1H), 1.44 (m, 2H), 1.36 (m, 0.5H), 1.32 (d, J=2.43 Hz, 1H), 1.28 (m, 2H), 1.24 (m, 1H), 0.98 (m, 1H) ppm. ¹³C NMR (75 MHz, CDCl3) δ 190.9, 145.6, 138.5, 130.1, 127.4, 57.1, 30.3, 28.7, 25.6, 25.2 ppm.

4-formyl-N-methyl-N-(oxan-4-yl)benzene-1-sulfonamide (23x). Compound 23x was synthesized using general procedure 4 and N-methyl-N-tetrahydro-2H-pyran-4-ylamine (22x). After purification the desired compound was afforded as a white amorphous soiled (16 mg, 19% yield) with a purity of 95% by UHPLC-MS. UHPLC-MS (ESI+APCI) m/z calcd. for C₁₃H₁₇NO₄S [M+H]⁺=284. Found: 254. Retention time: 1.35 min. ¹H NMR (300 MHz, CDCl3) δ 10.10 (s, 1H), 8.02 (d, J=8.61 Hz, 2H), 7.98 (d, J=8.56 Hz, 2H), 4.05 (m, 1H), 3.94 (dd, J=4.68 Hz, J=11.61 Hz, 2H), 3.39 (td, J=1.85 Hz, J=12.00 Hz, 2H), 2.80 (s, 3H), 1.70 (m, 2.5H), 1.38 (m, 2H), ppm. ¹³C NMR (75 MHz, CDCl3) δ 190.5, 145.1, 138.5, 130.0, 127.3, 66.9, 54.0, 30.0, 28.6 ppm.

4-(1,2,3,4-tetrahydroisoquinoline-2-sulfonyl)benzaldehyde (23y). Compound 23y was synthesized using general procedure 4 and 1,2,3,4-tetrahydroisoquinoline (22y). After purification the desired compound was afforded as a yellow amorphous soiled (40.2 mg, 45% yield) with a purity of 99% by UHPLC-MS. UHPLC-MS (ESI+APCI) m/z calcd. for C16H15NO3S [M+H]⁺=302. Found: 302. Retention time: 1.47 min. ¹H NMR (300 MHz, CDCl₃) δ 10.08 (s, 1H), 8.03 (d, J=8.63 Hz, 2H), 7.99 (d, J=8.65 Hz, 2H), 7.15 (m 2H), 7.04 (m 2H), 4.32 (s, 2H), 3.43 (t, 2H), 2.91 (m, 2H) ppm. ¹³C NMR (75 MHz, CDCl₃) δ 190.6, 141.8, 138.7, 132.6, 130.9, 130.0, 128.7, 128.0, 126.8, 126.3, 126.1, 47.2, 43.5, 28.5.

4-(1,2,3,4-tetrahydroquinoline-1-sulfonyl)benzaldehyde (23z). Compound 23z was synthesized using general procedure 4 and 1,2,3,4-tetrahydroquinoline (22z). After purification the desired compound was afforded as a white amorphous soiled (30 mg, 34% yield) with a purity of 99% by UHPLC-MS. UHPLC-MS (ESI+APCI) m/z calcd. for C₁₆H₁₅NO₃S [M+H]⁺=302. Found: 302. Retention time: 1.53 min. 1H NMR (300 MHz, CDCl3) δ 10.05 (s, 1H), 7.90 (d, J=8.54 Hz, 2H), 7.78 (dd, J=0.80 Hz, J=8.23 Hz, 2H), 7.74 (d, J=8.25 Hz, 2H), 7.21 (m, 1H), 7.10 (td, J=1.24 Hz, J=7.49 Hz, 1H), 7.00 (d, J=7.50 Hz, 1H), 3.84 (m, 2H), 2.41 (t, 2H), 1.64 (m, 2H) ppm. ¹³C NMR (75 MHz, CDCl3) δ 190.6, 144.4, 138.54, 136.0, 130.6, 129.8, 129.0, 127.5, 126.5, 125.3, 124.7, 46.5, 26.2, 21.5 ppm.

4-(2,3-dihydro-1H-indole-1-sulfonyl)benzaldehyde (24a). Compound 24a was synthesized using general procedure 4 and indoline. After purification the desired compound was afforded as an off-green amorphous soiled (22 mg, 26% yield) with a purity of 99% by UHPLC-MS. UHPLC-MS (ESI+APCI) m/z calcd. for _{C15H13NO3S} [M+H]⁺=288. Found: 288. Retention time: 1.46 min. ¹H NMR (300 MHz, CDCl3) δ 10.04 (s, 1H), 7.94 (s, 4H), 7.64 (d, J=8.10 Hz, 1H), 7.21 (td, J=7.38 Hz, J=0.66 Hz, 1H), 7.09 (dd, J=7.41 Hz, J=0.53 Hz, 1H), 7.00 (td, J=7.39 Hz, J=0.80 Hz, 1H), 3.96 (t, 2H), 2.90 (t, 2H) ppm. 13C NMR (75 MHz, CDCl3) δ 190.4, 141.8, 141.1, 138.9, 131.5, 129.8, 127.6, 127.6, 125.1, 124.0, 114.7, 49.8 and 27.6 ppm.

rac-4-[(2-methyl-1,2,3,4-tetrahydroquinolin-1-yl)sulfonyl]benzaldehyde (24b). Compound 24b was synthesized using general procedure 4 and 1,2,3,4-Tetrahydroquinaldine. After purification the desired compound was afforded as a brown amorphous soiled (10 mg, 15% yield) with a purity of 99% by UHPLC-MS. UHPLC-MS (ESI+APCI) m/z calcd. for C₁₇H₁₇NO₃S [M+H]⁺=316. Found: 316. Retention time: 1.59 min. ¹H NMR (300 MHz, CDCl3) δ 10.04 (s, 1H), 7.87 (d, J=8.45 Hz, 2H), 7.75 (d, J=8.08 Hz, 1H), 7.63 (d, J=8.27 Hz, 2H), 7.26, (d, J=15.49 Hz, 2H), 7.14 (td, J=7.48 Hz, J=1.08 Hz, 1H), 6.97 (d, J=7.29 Hz, 1H), 4.38 (q, 1H), 2.36 (m, 1H), 1.81 (m, 1H), 1.67 (m, 1H), 1.31 (d, J=6.52 Hz, 3H) ppm. ¹³C NMR (75 MHz, CDCl3) δ 190.8, 144.2, 138.7, 134.6, 133.6, 129.8, 128.1, 127.6, 127.5, 127.0, 126.1, 52.9, 30.4, 24.7 and 21.8 ppm.

4-[(6-fluoro-1,2,3,4-tetrahydroquinolin-1-yl)sulfonyl]benzaldehyde (24c). Compound 24c was synthesized using general procedure 4 and 6-Fluoro-1,2,3,4-tetrahydroquinoline. After purification the desired compound was afforded as a off-green amorphous soiled (22 mg, 25% yield) with a purity of 99% by UHPLC-MS. UHPLC-MS (ESI+APCI) m/z calcd. for C₁₆H₁₄FNO₃S [M+H]⁺=320. Found: 319. Retention time: 1.54 min. ¹H NMR (300 MHz, CDCl3) δ 10.06 (s, 1H), 7.91 (d, J=8.49 Hz, 2H), 7.76 (dd, J=9.08 Hz, J=5.23 Hz, 1H), 7.71 (d, J=8.31 Hz, 2H), 6.92 (td, J=8.85 Hz, J=3.01 Hz, 1H), 6.72 (dd, J=8.73 Hz, J=2.97 Hz, 1H), 3.81 (t, 2H), 2.35 (t, 2H), 1.59 (m, 2H) ppm. ¹³C NMR (75 MHz, CDCl3) δ 190.5, 161.7, 158.5, 144.2, 138.7, 133.2-133.1 (J=7.79 Hz), 132.02-131.98 (J=2.78 Hz), 129.9, 127.6, 127.07-126.96 (J=8.48 Hz), 115.4, 115.1, 113.8, 113.5, 46.4, 26.3 and 21.1 ppm.

4-[(6-chloro-1,2,3,4-tetrahydroquinolin-1-yl)sulfonyl]benzaldehyde (24d). Compound 24d was synthesized using general procedure 4 and 6-Chloro-1,2,3,4-tetrahydroquinoline. After purification the desired compound was afforded as a yellow amorphous soiled (17 mg, 17% yield) with a purity of 99% by UHPLC-MS. UHPLC-MS (ESI+APCI) m/z calcd. for C₁₆H₁₄ClNO₃S [M+H]⁺=336. Found: 335. Retention time: 1.64 min. ¹H NMR (300 MHz, CDCl₃) δ 10.06 (s, 1H), 7.92 (d, J=8.56 Hz, 2H), 7.75 (d, J=8.13 Hz, 3H), 7.18 (dd, J=8.82 Hz, J=2.51 Hz, 1H), 7.07 (d, J=2.43 Hz, 1H), 3.81 (t, 2H), 2.39 (t, 2H), 1.60 (m, 2H) ppm. ¹³C NMR (75 MHz, CDCl₃) δ 190.4, 144.1, 138.7, 134.7, 132.3, 10.8, 129.9, 128.8, 127.5, 126.7, 126.1, 46.4, 26.2 and 21.1 ppm.

4-[(6-methoxy-1,2,3,4-tetrahydroquinolin-1-yl)sulfonyl]benzaldehyde (24e). Compound 24e was synthesized using general procedure 4 and 6-methoxy-1,2,3,4-tetrahydroquinoline. After purification the desired compound was afforded as a off-green amorphous soiled (28 mg, 30% yield) with a purity of 99% by UHPLC-MS. UHPLC-MS (ESI+APCI) m/z calcd. for C₁₇H₁₇NO₄S [M+H]⁺=332. Found: 332. Retention time: 1.51 min. ¹H NMR (300 MHz, CDCl₃) δ 10.04 (s, 1H), 7.89 (d, J=8.04 Hz, 2H), 7.69 (d, J=7.33 Hz, 3H), 6.77 (d, J=8.85 Hz, 1H), 6.52 (s, 1H), 3.78 (s, 5H), 2.29 (t, 2H), 1.55 (t, 2H) ppm. ¹³C NMR (75 MHz, CDCl₃) δ 190.6, 157.1, 144.3, 138.4, 132.5, 129.6, 128.9, 127.5, 126.6, 113.5, 112.1, 55.1, 46.3, 26.2 and 21.2 ppm.

4-(3,4-dihydro-2H-1,4-benzoxazine-4-sulfonyl)benzaldehyde (24f). Compound 24f was synthesized using general procedure 4 and benzomorpholine. After purification the desired compound was afforded as a yellow amorphous soiled (18 mg, 20% yield) with a purity of 99% by UHPLC-MS. UHPLC-MS (ESI+APCI) m/z calcd. for C₁₅H₁₃NO₄S [M+H]⁺=304. Found: 303. Retention time: 1.46 min. ¹H NMR (300 MHz, CDCl₃) δ 10.06 (s, 1H), 7.94 (d, J=8.42 Hz, 2H), 7.84 (dd, J=8.25 Hz, J=1.52 Hz, 1H), 7.79 (d, J=8.33 Hz, 2H), 7.09 (td, J=7.44 Hz, J=1.56 Hz, 1H), 6.95 (td, J=8.51 Hz, J=1.54 Hz, 1H), 6.80 (dd, J=8.17 Hz, J=1.51 Hz, 1H), 3.92 (t, 2H), 3.74 (t, 2H) ppm. ¹³C NMR (75 MHz, CDCl₃) δ 190.5, 146.81, 143.7, 139.2, 130.2, 127.8, 126.8, 124.5, 123.3, 121.1, 117.7, 62.8 and 44.5 ppm.

4-[(6-fluoro-3,4-dihydro-2H-1,4-benzoxazin-4-yl)sulfonyl]benzaldehyde (24g). Compound 24g was synthesized using general procedure 4 and 6-Fluoro-3,4-dihydro-2H-benzo[1,4]oxazine. After purification the desired compound was afforded as a green amorphous soiled (13 mg, 15% yield) with a purity of 98% by UHPLC-MS. UHPLC-MS (ESI+APCI) m/z calcd. for C₁₅H₁₂FNO₄S [M+H]⁺=322. Found: 321. Retention time: 1.50 min. ¹H NMR (300 MHz, CDCl₃) δ 10.07 (s, 1H), 7.97 (d, J=8.57 Hz, 2H), 7.84 (d, J=8.34 Hz, 2H), 7.65 (dd, J=10.37 Hz, J=2.81 Hz, 1H), 6.78 (m, 2H), 3.91 (t, 2H), 3.73 (t, 2H) ppm. ¹³C NMR (75 MHz, CDCl₃) δ 190.4, 158.2, 155.0, 143.2, 142.84-142.80 (J=2.57 Hz), 139.4, 130.3, 127.9, 123.7-123.6 (J=10.74 Hz), 118.4-118.3 (J=8.89 Hz), 113.7, 113.4, 110.9, 110.5, 62.7 and 44.5 ppm.

4-[(7-chloro-3,4-dihydro-2H-1,4-benzoxazin-4-yl)sulfonyl]benzaldehyde (24h). Compound 24h was synthesized using general procedure 4 and 7-Chloro-3,4-dihydro-2H-benzo[b][1,4]oxazine. After purification the desired compound was afforded as a yellow amorphous soiled (10 mg, 10% yield) with a purity of 98% by UHPLC-MS. UHPLC-MS (ESI+APCI) m/z calcd. for C₁₅H₁₂ClNO₄S [M+H]⁺=338. Found: 337. Retention time: 1.30 min. ¹H NMR (300 MHz, CDCl₃) δ 10.07 (s, 1H), 7.97 (d, J=8.48 Hz, 2H), 7.79 (d, J=8.25 Hz, 2H), 7.79 (d, J=9.00 Hz, 1H), 6.94 (dd, J=8.88 Hz, J=2.40 Hz, 1H), 6.83 (d, J=2.38 Hz, 1H), 3.90 (t, 2H), 3.71 (t, 2H) ppm. ¹³C NMR (75 MHz, CDCl₃) δ 190.4, 147.3, 143.4, 139.3, 131.9, 130.4, 127.9, 125.6, 122.0, 121.4, 117.8, 62.8 and 44.3 ppm.

4-[(7-methoxy-3,4-dihydro-2H-1,4-benzoxazin-4-yl)sulfonyl]benzaldehyde (24i). Compound 24i was synthesized using general procedure 4 and 7-Methoxy-3,4-dihydro-2H-benzo[b][1,4]oxazine. After purification the desired compound was afforded as a brown amorphous soiled (16.5 mg, 17% yield) with a purity of 99% by UHPLC-MS. UHPLC-MS (ESI+APCI) m/z calcd. for C₁₆H₁₅NO₅S [M+H]⁺=334. Found: 334. Retention time: 1.46 min. ¹H NMR (300 MHz, CDCl₃) δ 10.06 (s, 1H), 7.94 (d, J=8.04 Hz, 2H), 7.75 (d, J=7.89 Hz, 2H), 7.74 (d, J=6.02 Hz, 2H), 6.55 (dd, J=9.09 Hz, J=2.43, Hz, 1H), 6.32 (d, J=2.35, Hz, 1H), 3.87 (t, 2H), 3.76 (s, 3H), 3.64 (t, 2H) ppm. ¹³C NMR (75 MHz, CDCl₃) δ 190.6, 158.6, 147.9, 143.6, 139.1, 130.2, 127.9, 126.1, 116.2, 107.9, 102.0, 62.6, 55.4 and 44.4 ppm.

4-(1,2,3,4-tetrahydroquinoxaline-1-sulfonyl)benzaldehyde (24j). Compound 24i was synthesized using general procedure 4 and 1,2,3,4-Tetrahydro-quinoxaline. After purification the desired compound was afforded as a yellow oil (31.2 mg, 40% yield) with a purity of 98% by UHPLC-MS. UHPLC-MS (ESI+APCI) m/z calcd. for C15H₁₄N₂O₃S [M+H]⁺=303. Found: 303. Retention time: 1.37 min. ¹H NMR (300 MHz, CDCl₃) δ 10.03 (s, 1H), 7.89 (d, J=8.54 Hz, 2H), 7.71 (d, J=8.30 Hz, 2H), 7.64 (dd, J=8.27 Hz, J=1.33 Hz, 1H), 7.00 (td, J=8.13 Hz, J=1.41 Hz, 1H), 6.70 (td, J=8.31 Hz, J=1.38 Hz, 1H), 6.45 (dd, J=8.05 Hz, J=1.24 Hz, 1H), 3.83 (t, 3H), 2.94 (t, 2H), 2.00 (s, 1H) ppm. ¹³C NMR (75 MHz, CDCl₃) δ 190.4, 144.2, 138.4, 137.3, 136.3, 129.6, 127.4, 126.7, 125.7, 120.6, 116.8, 114.3, 111.2, 43.5 and 38.6 ppm.

### Example 13: Additional Compounds and Data

TABLE 10

| | chloroacetamides | | | | disulfides | | | |
|---|---|---|---|---|---|---|---|---|
| | Raf | | ER | | Raf | | ER | |
| | | Fp | | Fp | | Fp | | Fp |
| | | (EC₅₀), | | (EC₅₀), | | (EC₅₀), | | (EC₅₀), |
| | | μM/fold- | | μM/fold- | | μM/fold- | | μM/fold- |
| | MS | stabiliza- | MS | stabiliza- | MS | stabiliza- | MS | stabiliza- |
| | (K_{D,app}) | tion of | (K_{D,app}), | tion of | (K_{D,app}) | tion of | (K_{D,app}), | tion of |
| structure | μM | peptide | μM | peptide | μM | peptide | μM | peptide |
| 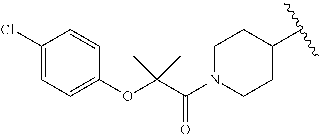 [SMILES: *C1CCN(C(=O)C(C)(C)Oc2ccc(Cl)cc2)CC1] | 17.8 | NA | 84.3 | 38.9 | 1.91 | 7.8/155 | 51.4 | 1.15/20 |
| 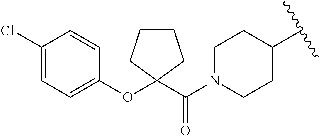 [SMILES: *C1CCN(C(=O)C2(Oc3ccc(Cl)cc3)CCCC2)CC1] | 0.19 | 11.4 | 4.1 | 33 | NA | NA | NA | NA |
| 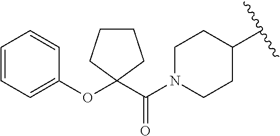 [SMILES: *C1CCN(C(=O)C2(Oc3ccccc3)CCCC2)CC1] | 0.082 | 13/69 | 105 | 37.6/33 | 14.4 | NA | 33.3 | NA |
| 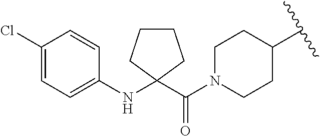 [SMILES: *C1CCN(C(=O)C2(Nc3ccc(Cl)cc3)CCCC2)CC1] | 0.15 | 66.1 | ~0.1 | 8.1/23 | 115 | NA | 6.41 | NA |
| 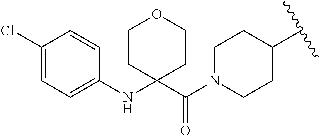 [SMILES: *C1CCN(C(=O)C2(Nc3ccc(Cl)cc3)CCOCC2)CC1] | 19.2 | NA/2-3 | 2.8 | 3.5/20-40 | 300 | NA | 5.73 | NA |
| 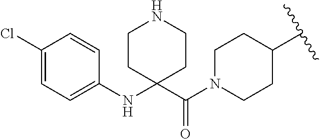 [SMILES: *C1CCN(C(=O)C2(Nc3ccc(Cl)cc3)CCNCC2)CC1] | NA | NA | <0.1 | 7.3 | 720 | NA | 0.604 | NA |
| 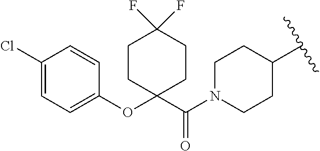 [SMILES: *C1CCN(C(=O)C2(Oc3ccc(Cl)cc3)CCC(F)(F)CC2)CC1] | 1.2 | 27.1 | 3.1 | 41.3 | 0.545 | NA | 16.4 | NA |

TABLE 11

| | Raf | | ER | |
|---|---|---|---|---|
| | MS (K_{D,app}), | Fp (EC₅₀), | MS (K_{D,app}), | Fp (EC₅₀), |
| | μM, 24 hr | μM, 24 hr | μM, 24 hr | μM, 24 hr |
| structure | incubation | incubation | incubation | incubation |
| 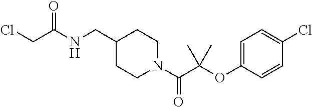 [SMILES: CC(C)(Oc1ccc(Cl)cc1)C(=O)N1CCC(CNC(=O)CCl)CC1] | 17.8 | NA | 84.3 | 38.9 |
| 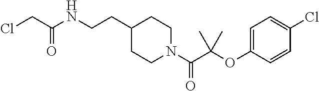 [SMILES: CC(C)(Oc1ccc(Cl)cc1)C(=O)N1CCC(CCNC(=O)CCl)CC1] | 15.4 | 20.5 | 33.7 | 32.7 |
| 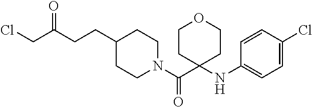 [SMILES: O=C(CCl)CCC1CCN(C(=O)C2(Nc3ccc(Cl)cc3)CCOCC2)CC1] | ~0.10 | NA | 0.145 | NA |
| 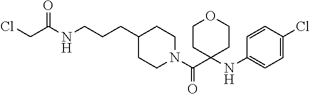 [SMILES: O=C(CCl)NCCCC1CCN(C(=O)C2(Nc3ccc(Cl)cc3)CCOCC2)CC1] | NA | NA | <0.1 | NA |
| 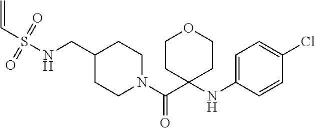 [SMILES: C=CS(=O)(=O)NCC1CCN(C(=O)C2(Nc3ccc(Cl)cc3)CCOCC2)CC1] | NA | NA | 0.125 | NA |
| 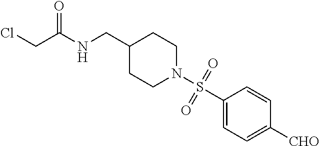 [SMILES: O=Cc1ccc(S(=O)(=O)N2CCC(CNC(=O)CCl)CC2)cc1] | 181 | NA | 150 | NA |
| 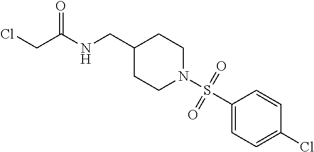 [SMILES: O=C(CCl)NCC1CCN(S(=O)(=O)c2ccc(Cl)cc2)CC1] | NA | NA | 7.15 | NA |

### REFERENCES

(1) Lee, C.-H.; et al. Proc. Natl. Acad. Sci. U.S.A 2012, 109 (3), 929-934. (2) Suzuki, S.; et al. Pathol. Res. Pract. 2014, 210 (2), 130-134. (3) Lee, C.-H.; et al. Am. J. Surg. Pathol. 2012, 36 (5), 641-653. (4) Gremel, G.; et al. Int. J. Cancer 2015, 136 (7), 1608-1618. (5) Attygalle, A. D.; Vroobel, K.; Wren, D.; Barton, D. P. J.; Hazell, S. J.; Cin, P. D.; Koelble, K.; McCluggage, W. G. Int. J. Gynecol. Pathol. Off. J. Int. Soc. Gynecol. Pathol. 2017, 36 (2), 165-171. (6) Hemming, M. L.; et al. Gynecol. Oncol. 2017, 145 (3), 531-535. (7) Lee, C.-H.; et al. Mod. Pathol. Off. J. U. S. Can. Acad. Pathol. Inc 2014, 27 (5), 751-757. (8) Lee, C.-H.; et al. Mod. Pathol. Off. J. U. S. Can. Acad. Pathol. Inc 2014, 27 (5), 751-757. (9) Masone, D.; et al. Sci. Rep. 2017, 7, 46114. (10) Ganguly, S.; et al. Proc. Natl. Acad. Sci. U.S.A. 2005, 102 (4), 1222-1227. (11) Ganguly, S.; et al. Proc. Natl. Acad. Sci. U.S.A 2001, 98 (14), 8083-8088. (12) Obsil, T.; et al. Cell 2001, 105 (2), 257-267. (13) Pozdeyev, N.; et al. J. Neurosci. Off. J. Soc. Neurosci. 2006, 26 (36), 9153-9161. (14) Klein, D. C.; et al. Biochem. Soc. Trans. 2002, 30 (4), 365-373. (15) Schomerus, C.; et al. Acad. Sci. 2005, 1057, 372-383. (16) Huang, Z.; et al. J. Pineal Res. 2009, 46 (1), 119-120. (17) Pagan, C.; et al. Sci. Rep. 2017, 7 (1), 2096. (18) Adler, J. J.; et al. Proc. Natl. Acad. Sci. U.S.A 2013, 110 (43), 17368-17373. (19) Ramm, G.; et al. J. Biol. Chem. 2006, 281 (39), 29174-29180. (20) Howlett, K. F.; et al. Diabetes 2007, 56 (6), 1608-1614. (21) Geraghty, K. M.; et al. Biochem. J. 2007, 407 (2), 231-241. (22) Chen, S.; et al. Biochem. J. 2008, 409 (2), 449-459. (23) Ngo, S.; et al. Mol. Cell. Endocrinol. 2009, 302 (1), 33-40. (24) Chen, S.; et al. Cell Metab. 2011, 13 (1), 68-79. (25) Koumanov, F.; et al. J. Biol. Chem. 2011, 286 (19), 16574-16582. (26) Tan, S.-X.; et al. Mol. Cell. Biol. 2012, 32 (24), 4946-4959. (27) The effect of exercise and insulin on AS160 phosphorylation and 14-3-3 binding capacity in human skeletal muscle.—PubMed—NCBI https://www.ncbi.nlm.nih.gov/pubmed/18042670 (accessed Dec. 18, 2018). (28) Disruption of AMPKalpha1 signaling prevents AICAR-induced inhibition of AS160/TBC1D4 phosphorylation and glucose uptake in primary rat adipocytes.—PubMed—NCBI https://www.ncbi.nlm.nih.gov/pubmed/20501641 (accessed Dec. 18, 2018). (29) Treebak, J. T.; et al. Diabetologia 2009, 52 (5), 891-900. (30) Cockrell, L. M.; et al. Oncogene 2010, 29 (6), 822-830. (31) Brobey, R. K.; et al. PloS One 2015, 10 (10), e0141968. (32) Lee, J. A.; et al. Oncogene 2008, 27 (9), 1297-1305. (33) Min, W.; et al. Circ. Res. 2008, 102 (7), 840-848. (34) Obsil, T.; et al. Adv. Biol. Regul. 2017, 66, 31-36. (35) Petrvalska, O.; et al. J. Biol. Chem. 2016, 291 (39), 20753-20765. (36) Thandavarayan, R. A.; et al. Biochem. Pharmacol. 2008, 75 (9), 1797-1806. (37) Wang, P.; et al. Neurosci. Lett. 2007, 415 (3), 248-252. (38) Zheng, S.; et al. Cardiovasc. Pharmacol. Open Access 2015, 4 (1). (39) Moore, A. E.; et al. Wiley Interdiscip. Rev. RNA 2014, 5 (4), 549-564. (40) White, E. J. F. et al. Wiley Interdiscip. Rev. RNA 2017, 8 (2). (41) He, C.; Schneider, R. EMBO J. 2006, 25 (16), 3823-3831. (42) Fu, H.; et al. Science 1994, 266 (5182), 126-129. (43) Fantd, W. J.; et al. Nature 1994, 371 (6498), 612-614. (44) Freed, E.; et al. Cold Spring Harb. Symp. Quant. Biol. 1994, 59, 187-193. (45) Freed, E.; et al. Science 1994, 265 (5179), 1713-1716. (46) Irie, K.; et al. Science 1994, 265 (5179), 1716-1719. (47) Li, S.; et al. EMBO J. 1995, 14 (4), 685-696. (48) Rommel, C.; et al. Oncogene 1996, 12 (3), 609-619. (49) Tzivion, G.; et al. Nature 1998, 394 (6688), 88-92. (50) Light, Y.; et al. Mol. Cell. Biol. 2002, 22 (14), 4984-4996. (51) Dumaz, N.; Marais, R. J. Biol. Chem. 2003, 278 (32), 29819-29823. (52) Garnett, M. J.; et al. Mol. Cell 2005, 20 (6), 963-969. (53) Kasukabe, T.; et al. Breast Cancer Res. BCR 2005, 7 (6), R1097-1110. (54) Baljuls, A.; et al. J. Biol. Chem. 2008, 283 (40), 27239-27254. (55) Fischer, A.; et al. J. Biol. Chem. 2009, 284 (5), 3183-3194. (56) Molzan, M.; et al. Mol. Cell. Biol. 2010, 30 (19), 4698-4711. (57) Molzan, M.; et al. J. Mol. Biol. 2012, 423 (4), 486-495. (58) Shen, C.-H.; et al. Mol. Cell 2013, 52 (2), 161-172. (59) Desideri, E.; et al. Cell 2015, 161 (5), 967-970. (60) Fan, J.; et al. Neurosci. Lett. 2010, 473 (3), 196-201. (61) Fan, J.; et al. Int. J. Neurosci. 2012, 122 (11), 665-674. (62) Han, J. Y.; et al. J. Neurosci. Res. 2008, 86 (14), 3221-3229. (63) Koh, P.-O. J. Pineal Res. 2008, 44 (1), 101-106. (64) Koh, P.-O. Neurosci. Lett. 2011, 498 (2), 105-109. (65) Koh, P.-O. Neurosci. Lett. 2012, 507 (2), 156-160. (66) Meller, R.; et al. Cell Death Differ. 2003, 10 (5), 539-547. (67) RAcz, B.; et al. Neurotox. Res. 2007, 12 (2), 95-104. (68) Springer, J. E.; et al. J. Neurosci. Off. J. Soc. Neurosci. 2000, 20 (19), 7246-7251. (69) Wang, H. G.; et al. Science 1999, 284 (5412), 339-343. (70) Yu, F.; et al. Neurobiol. Dis. 2005, 20 (2), 491-499. (71) Zha, J.; et al. Cell 1996, 87 (4), 619-628. (72) Won, C. K.; et al. Neurosci. Lett. 2005, 387 (2), 115-119. (73) Ge, F.; et al. J. Proteome Res. 2010, 9 (11), 5848-5858. (74) Xu, Z.; et al. J. Cell Sci. 2013, 126 (Pt 18), 4173-4186. (75) Jia, B.; et al. Prion 2014, 8 (2). (76) Sime, W.; et al. Cell Death Dis. 2018, 9 (5), 458. (77) Kim, J. A.; et al. BMB Rep. 2017, 50 (5), 257-262. (78) Lee, S. K.; et al. Biochem. Biophys. Res. Commun. 2007, 352 (3), 756-762. (79) Nomura, M.; et al. J. Biol. Chem. 2003, 278 (3), 2058-2065. (80) Samuel, T.; et al. J. Biol. Chem. 2001, 276 (48), 45201-45206. (81) Slone, S. R.; et al. PloS One 2011, 6 (7), e21720. (82) Tsuruta, F.; et al. EMBO J. 2004, 23 (8), 1889-1899. (83) Shinoda, S.; et al. J. Neurochem. 2003, 86 (2), 460-469. (84) Schindler, C. K.; et al. Neurosci. Lett. 2004, 356 (3), 163-166. (85) Qi, X.-J.; et al. J. Biol. Chem. 2006, 281 (2), 813-823. (86) Wang, X.; et al. J. Biol. Chem. 2012, 287 (14), 11037-11048. (87) Mohammad, D. K.; et al. Int. J. Biochem. Cell Biol. 2016, 78, 63-74. (88) Mohammad, D. K.; et al. Mol. Cell. Biol. 2013, 33 (16), 3214-3226. (89) Psenakova, K.; et al. Biochim. Biophys. Acta Gen. Subj. 2018, 1862 (7), 1612-1625. (90) Li, Y.; et al. Neuron 2006, 51 (6), 755-771. (91) Li, Y.; et al. Channels Austin Tex 2007, 1 (1), 1-2. (92) Liu, F.; et al. J. Biol. Chem. 2015, 290 (5), 2689-2698. (93) Andersen, J. L.; et al. Mol. Cell 2011, 43 (5), 834-842. (94) Kalabova, D.; et al. Biochem. Biophys. Res. Commun. 2017, 493 (2), 940-945. (95) Nutt, L. K.; et al. Dev. Cell 2009, 16 (6), 856-866. (96) Smidova, A.; et al. FEBS J. 2018, 285 (22), 4196-4213. (97) Conklin, D. S.; et al. 1 Proc. Natl. Acad. Sci. U.S.A 1995, 92 (17), 7892-7896. (98) Peng, C. Y.; et al. Science 1997, 277 (5331), 1501-1505. (99) Sanchez, Y.; et al. Science 1997, 277 (5331), 1497-1501. (100) Zeng, Y.; et al. Nature 1998, 395 (6701), 507-510. (101) Kumagai, A.; et al. Mol. Biol. Cell 1998, 9 (2), 345-354. (102) Dalal, S. N.; Schweitzer, C. M.; Gan, J.; DeCaprio, J. et al. Mol. Cell. Biol. 1999, 19 (6), 4465-4479. (103) Kumagai, A.; et al. Genes Dev. 1999, 13 (9), 1067-1072. (104) Lopez-Girona, A.; etc. Nature 1999, 397 (6715), 172-175. (105) Yang, J.; et al. EMBO J. 1999, 18 (8), 2174-2183. (106) Davezac, N.; et al. Oncogene 2000, 19 (18), 2179-2185. (107) Mils, V.; et al. Oncogene 2000, 19 (10), 1257-1265. (108) Morris, M. C.; et al. J. Biol. Chem. 2000, 275 (37), 28849-28857. (109) Forrest, A.; et al. Oncogene 2001, 20 (32), 4393-4401. (110) Graves, P. R.; et al. Oncogene 2001, 20 (15), 1839-1851. (111) Bulavin, D. V.; et al. Nat. Cell Biol. 2003, 5 (6), 545-551. (112) Chen, M.-S.; et al. Mol. Cell. Biol. 2003, 23 (21), 7488-7497. (113) Giles, N.; et al. J. Biol. Chem. 2003, 278 (31), 28580-28587. (114) Margolis, S. S.; et al. EMBO J. 2003, 22 (21), 5734-5745. (115) Uchida, S.; et al. Biochem. Biophys. Res. Commun. 2004, 316 (1), 226-232. (116) Margolis, S. S.; et al. Role for the PP2A/B56delta Phosphatase in Regulating 14-3-3 Release from Cdc25 to Control Mitosis. Cell 2006, 127 (4), 759-773. (117) Chan, P. M.; et al. Mol. Cell. Proteomics MCP 2011, 10 (3), M110.005157. (118) Zhang, Z.; et al. Tumour Biol. J. Int. Soc. Oncodevelopmental Biol. Med. 2014, 35 (3), 2503-2512. (119) Cho, Y.-C.; et al. Cell Death Differ. 2015, 22 (10), 1605-1617. (120) Laronga, C.; et al. J. Biol. Chem. 2000, 275 (30), 23106-23112. (121) Liang, X.; et al. Mol. Biol. Cell 2012, 23 (6), 996-1009. (122) Stevers, L. M.; et al. Proc. Natl. Acad. Sci. U.S.A 2016, 113 (9), E1152-1161. (123) Bozoky, Z.; et al. Proc. Natl. Acad. Sci. U.S.A 2013, 110 (47), E4427-4436. (124) Ciavardelli, D.; et al. Mol. Biosyst. 2013, 9 (6), 1117-1126. (125) Stevers, L. M.; et al. J. Am. Chem. Soc. 2018, 140 (43), 14498-14510. (126) Li, F.-Q.; et al. J. Cell Biol. 2008, 181 (7), 1141-1154. (127) Takemaru, K.-I.; et al. Cell Cycle Georget. Tex 2009, 8 (2), 210-213. (128) Li, F.-Q.; et al. Mol. Biol. Cell 2010, 21 (2), 311-322. (129) Mancini, M.; et al. Cell. Signal. 2013, 25 (9), 1820-1827. (130) Killoran, R. C.; et al. PloS One 2015, 10 (4), e0123934. (131) Mancini, M.; et al. PloS One 2015, 10 (7), e0131074. (132) Mancini, M.; et al. Oncotarget 2017, 8 (50), 88244-88250. (133) Fischer, V.; et al. J. Mol. Signal. 2012, 7 (1), 6. (134) Chen, L.; et al. Genes Dev. 1999, 13 (6), 675-685. (135) Hapke, G.; et al. Int. J. Oncol. 2002, 21 (5), 1059-1066. (136) Jiang, K.; Pereira, E.; Maxfield, M.; Russell, B.; Goudelock, D. M.; Sanchez, Y. J. Biol. Chem. 2003, 278 (27), 25207-25217. (137) Ashra, H.; Rao, K. V. K. Cancer Lett. 2006, 237 (2), 188-198. (138) Sakiyama, H.; et al. J. Biol. Chem. 2008, 283 (36), 24899-24908. (139) Ge, Q.; et al. J. Biol. Chem. 2011, 286 (32), 28119-28127. (140) Nakagawa, T.; et al. J. Biol. Chem. 2013, 288 (39), 28358-28367. (141) Ge, Q.; et al. J. Biol. Chem. 2012, 287 (50), 41914-41921. (142) Abdul-Wahed, A.; et al. Cell Metab. 2017, 26 (2), 324-341. (143) Prescott, G. R.; Jenkins, R. E.; Walsh, C. M.; Morgan, A. Biochem. Biophys. Res. Commun. 2008, 377 (3), 809-814. (144) Patel, P.; et al. Struct. Lond. Engl. 1993 2016, 24 (8), 1380-1386. (145) Shirafuji, T.; et al. J. Neurosci. Off. J. Soc. Neurosci. 2018, 38 (2), 278-290. (146) Fukui-Miyazaki, A.; et al. mBio 2018, 9 (4). (147) Wang, B.; et al. J. Biol. Chem. 2004, 279 (52), 54140-54152. (148) Milton, A. H.; et al. EMBO J. 2006, 25 (5), 1046-1057. (149) De Vries-van Leeuwen, I. J.; et al. Proc. Natl. Acad. Sci. U.S.A. 2013, 110 (22), 8894-8899. (150) Fu, H.; et al. Proc. Natl. Acad. Sci. U.S.A 1993, 90 (6), 2320-2324. (151) Masters, S. C.; et al. Biochemistry 1999, 38 (16), 5216-5221. (152) Zhang, L.; et al. Biochemistry 1999, 38 (37), 12159-12164. (153) Henriksson, M. L.; et al. Biochem. J. 2000, 349 Pt 3, 697-701. (154) Riese, M. J.; et al. J. Biol. Chem. 2002, 277 (14), 12082-12088. (155) Hallberg, B. Biochem. Soc. Trans. 2002, 30 (4), 401-405. (156) Henriksson, M. L.; et al. Eur. J. Biochem. 2002, 269 (20), 4921-4929. (157) Pederson, K. J.; et al. Mol. Microbiol. 2002, 46 (5), 1381-1390. (158) Krall, R.; et al. J. Biol. Chem. 2004, 279 (4), 2747-2753. (159) Zhang, Y.; et al. Infect. Immun. 2005, 73 (12), 7938-7945. (160) Yasmin, L.; et al. FEBS J. 2006, 273 (3), 638-646. (161) Maresso, A. W.; et al. Cell. Microbiol. 2007, 9 (1), 97-105. (162) Ottmann, C.; et al. EMBO J. 2007, 26 (3), 902-913. (163) Zhang, Y.; et al. J. Biol. Chem. 2007, 282 (17), 13022-13032. (164) Zhang, Y.; et al. Cell. Microbiol. 2007, 9 (9), 2192-2201. https://doi.org/10.1111/j.1462-5822.2007.00949.x. (165) Yasmin, L.; et al. Biochem. J. 2010, 427 (2), 217-224. (166) Bier, D.; et al. Nat. Chem. 2013, 5 (3), 234-239. (167) Okuda, J.; et al. Infect. Chemother. Off. J. Jpn. Soc. Chemother. 2014, 20 (2), 121-127. (168) Pinto, A. F.; et al. J. Biomol. Screen. 2016, 21 (6), 590-595. (169) Karlberg, T.; et al. Nat. Commun. 2018, 9 (1), 3785. (170) Glas, A.; et al. Angew. Chem. Int. Ed Engl. 2014, 53 (9), 2489-2493. (171) Cromm, P. M.; et al. Chembiochem Eur. J. Chem. Biol. 2016, 17 (20), 1915-1919. (172) Kruger, D. M.; et al. J. Med. Chem. 2017, 60 (21), 8982-8988. (173) Karlberg, T.; H; et al. Nat. Commun. 2018, 9 (1), 3785. (174) Zhao, X; et al. Cell 2006, 125 (5), 987-1001. (178) Yuan, Z.; et al. J. Biol. Chem. 2009, 284 (17), 11285-11292. (179) Su, Y.-W.; et al. Proc. Natl. Acad. Sci. U.S.A 2011, 108 (4), 1555-1560. (180) Oh, H.-M.; et al. J. Biol. Chem. 2012, 287 (36), 30436-30443. (181) Daitoku, H.; et al. Biochim. Biophys. Acta 2011, 1813 (11), 1954-1960. (182) Gerst, F.; et al. Diabetologia 2015, 58 (12), 2819-2831. (183) Schall, D.; et al. Eur. J. Immunol. 2015, 45 (11), 3087-3097. (184) Weng, Q.; et al. PloS One 2016, 11 (12), e0167869. (185) Brummer, T.; et al. EMBO J. 2008, 27 (17), 2305-2316. (186) Wöhrle, F. U.; et al. Leukemia 2013, 27 (1), 118-129. (187) Herrera Abreu, M. T.; et al. J. Mol. Biol. Cell 2011, 22 (1), 105-116. (188) Bier, D.; et al. ChemMedChem 2016, 11 (8), 911-918. (189) Yoshimura, Y.; Terabayashi, T.; Miki, H. Mol. Cell. Biol. 2010, 30 (9), 2206-2219. (190) Asaoka, Y.; et al. J. Biol. Chem. 2010, 28 et al. J. Neurovirol. 2008, 14 (3), 186-195. (193) Kapasi, A. A.; et al. Am. J. Physiol. Renal Physiol. 2001, 280 (2), F333-342. (194) Rong, J.; et al. J. Biol. Chem. 2007, 282 (7), 4748-4756. (195) Diao, J.; et al. J. Biol. Chem. 2001, 276 (11), 8328-8340. (196) Aoki, H.; et al. Virol. 2000, 74 (4), 1736-1741. (197) et al. J. Virol. 2010, 84 (12), 5936-5946. (199) Nakamura, H.; et al. Hepatol. Res. Off. J. Jpn. Soc. Hepatol. 2011, 41 (5), 455-462. (200) Grozinger, C. M.; et al. Proc. Natl. Acad. Sci. U.S.A. 2000, 97 (14), 7835-7840. (201) McKinsey, T. A.; et al. Proc. Natl. Acad. Sci. U.S.A. 2000, 97 (26), 14400-14405. (202) Zhang, C. L.; et al. N. Proc. Natl. Acad. Sci. U.S.A. 2001, 98 (13), 7354-7359. (203) Fischle, W.; et al. Biochem. Cell Biol. Biochim. Biol. Cell. 2001, 79 (3), 337-348. (204) Identification of a signal-responsive nuclear export sequence in class II histone deacetylases.—PubMed—NCBI https://www.ncbi.nlm.nih.gov/pubmed/11509672 (accessed Dec. 18, 2018). (205) Kao, H. Y.; et al. J. Biol. Chem. 2001, 276 (50), 47496-47507. (206) Ellis, J. J.; et al. Mol. Cell. Biochem. 2003, 242 (1-2), 153-161. (207) Chang, S.; et al. N. Proc. Natl. Acad. Sci. U.S.A. 2005, 102 (23), 8120-8125. (208) Nishino, T. G.; et al. Biochem. Biophys. Res. Commun. 2008, 377 (3), 852-856. (209) Harrison, B. C.; et al. FEBS Lett. 2010, 584 (6), 1103-1110. (210) Rajendran, P.; et al. Mol. Cancer 2011, 10, 68. (211) Keum, Y.-S.; et al. Oncogene 2013, 32 (4), 444-452. (212) Healy, S.; et al. Discov. Med. 2011, 11 (59), 349-358. (213) Sawicka, A.; et al. Biochim. Biophys. Acta 2014, 1839 (8), 711-718. (214) Zippo, A.; et al. Cell 2009, 138 (6), 1122-1136. (215) Winter, S.; et al. EMBO J. 2008, 27 (1), 88-99. (216) Walter, W.; et al. Mol. Cell. Biol. 2008, 28 (8), 2840-2849. (217) Macdonald, N.; et al. Mol. Cell 2005, 20 (2), 199-211. (218) Wang, X.; et al. Mol. Cell. Biol. 2003, 23 (17), 6013-6026. (219) Wang, X.; et al. J. Biol. Chem. 2004, 279 (47), 49460-49469. (220) Chernik, I. S.; et al. Mol. Cell. Biochem. 2007, 295 (1-2), 9-17. (221) Seit-Nebi, A. S.; et al. Cell Stress Chaperones 2010, 15 (3), 233-236. (222) Sluchanko, N. N.; et al. Arch. Biochem. Biophys. 2011, 506 (1), 24-34. (223) Sluchanko, N. N.; et al. Biochemistry 2011, 50 (45), 9797-9808. (224) Sluchanko, N. N.; et al. Biochemistry 2012, 51 (31), 6127-6138. (225) Sudnitsyna, M. V.; et al. Arch. Biochem. Biophys. 2012, 521 (1-2), 62-70. (226) Sluchanko, N. N.; et al. Struct. Lond. Engl. 1993 2017, 25 (2), 305-316. (227) Omi, K.; et al. Neurosci. Lett. 2008, 431 (1), 45-50. (228) Bonet, R.; et al. J. Mol. Biol. 2013, 425 (17), 3060-3072. (229) Fagerholm, S. C.; et al. J. Cell Biol. 2005, 171 (4), 705-715. (230) Nurmi, S. M.; et al. J. Biol. Chem. 2007, 282 (2), 968-975. (231) Jahan, F.; et al. Phosphorylation of the α-Chain in the Integrin LFA-1 J. Biol. Chem. 2018, 293 (32), 12318-12330. (232) Camoni, L.; et al. Biochem. J. 2011, 436 (2), 429-436. (233) Craparo, A.; et al. J. Biol. Chem. 1997, 272 (17), 11663-11669. (234) Furlanetto, R. W.; et al. Biochem. J. 1997, 327 (Pt 3), 765-771. (235) Lopaczynski, W. Acta Biochim. Pol. 1999, 46 (1), 51-60. (236) Peruzzi, F.; et al. J. Biol. Chem. 2001, 276 (28), 25990-25996. (237) Parvaresch, S.; et al. FEBS Lett. 2002, 532 (3), 357-362. (238) Spence, S. L.; et al. Biochem. Biophys. Res. Commun. 2003, 312 (4), 1060-1066. (239) Stomski, F. C.; et al. Blood 1999, 94 (6), 1933-1942. (240) Guthridge, M. A.; et al. Mol. Cell 2000, 6 (1), 99-108. (241) Sliva, D.; et al. Biochem. J. 2000, 345 Pt 3, 741-747. (242) Paiardini, A.; et al. IUBMB Life 2014, 66 (1), 52-62. (243) Aguilera, C.; et al. J. Cell Sci. 2006, 119 (Pt 17), 3695-3704. (244) Kast, D. J.; et al. Nat. Commun. 2019, 10 (1), 483. (245) Ogihara, T.; et al. J. Biol. Chem. 1997, 272 (40), 25267-25274. (246) Kosaki, A.; et al. J. Biol. Chem. 1998, 273 (2), 940-944. (247) Federici, M.; et al. FASEB J. Off. Publ. Fed. Am. Soc. Exp. Biol. 2001, 15 (1), 22-24. (248) Xiang, X.; et al. Mol. Endocrinol. Baltim. Md 2002, 16 (3), 552-562. (249) Moeschel, K.; et al. J. Biol. Chem. 2004, 279 (24), 25157-25163. (250) Oriente, F.; et al. J. Biol. Chem. 2005, 280 (49), 40642-40649. (251) Raychaudhuri, K.; et al. J. Biol. Chem. 2016, 291 (31), 16068-16081. (252) Xing, H.; et al. Curr. Biol. CB 1997, 7 (5), 294-300. (253) Cacace, A. M.; et al. Mol. Cell. Biol. 1999, 19 (1), 229-240. (254) Ory, S.; et al. Curr. Biol. CB 2003, 13 (16), 1356-1364. (255) Müller, J.; et al. Mol. Cell 2001, 8 (5), 983-993. (256) Jagemann, L. R.; et al. J. Biol. Chem. 2008, 283 (25), 17450-17462. (257) Shen, C.-H.; et al. Mol. Cell 2013, 52 (2), 161-172. (258) Takahashi, M.; et al. J. Biol. Chem. 2017, 292 (4), 1449-1461. (259) Ory, S.; et al. Curr. Biol. CB 2003, 13 (16), 1356-1364. (260) Jagemann, L. R.; et al. J. Biol. Chem. 2008, 283 (25), 17450-17462. (261) Shao, Z.; et al. Sci. Rep. 2016, 6, 25631. (262) Choi, E. Y.; et al. Blood 2008, 111 (7), 3607-3614. (263) Granholm, M.; et al. J. Immunol. Baltim. Md 1950 2011, 187 (7), 3613-3619. (264) Granholm, M.; et al. Blood 2016, 128 (9), 1270-1281. (265) Al-Hakim, A. K.; et al. J. Cell Sci. 2005, 118 (Pt 23), 5661-5673. (266) Ding, S.; et al. Acta Crystallogr. Sect. F Struct. Biol. Commun. 2015, 71 (Pt 9), 1114-1119. (267) Liu, L.; et al. Am. J. Transl. Res. 2012, 4 (2), 175-186. (268) Doggett, E. A.; et al. J. Neurochem. 2012, 120 (1), 37-45. (269) Dzamko, N.; et al. PloS One 2012, 7 (6), e39132. (270) Dzamko, N.; et al. J. Park. Dis. 2013, 3 (2), 145-152. (271) Fraser, K. B.; et al. Hum. Mol. Genet. 2013, 22 (24), 4988-5000. (272) Lavalley, N. J.; et al. Hum. Mol. Genet. 2016, 25 (1), 109-122. (273) Li, X.; et al. PloS One 2011, 6 (3), e17153. (274) Long, S.; et al. Front. Neurosci. 2018, 12, 782. (275) Mamais, A.; et al. J. Biol. Chem. 2014, 289 (31), 21386-21400. (276) Melrose, H. L. Biochem. J. 2015, 470 (3), e21-24. (277) Muda, K.; et al. Proc. Natl. Acad. Sci. U.S.A 2014, 111 (1), E34-43. (278) Nichols, R. J.; et al. Biochem. J. 2010, 430 (3), 393-404. (279) Reyniers, L.; et al. J. Neurochem. 2014, 131 (2), 239-250. (280) Rudenko, I. N.; et al. Biochem. J. 2010, 430 (3), e5-6. (281) Stevers, L. M.; et al. Biochem. J. 2017, 474 (7), 1273-1287. (282) Zhao, J.; et al. Biochem. J. 2015, 469 (1), 107-120. (283) Zhao, Y.; et al. Neurobiol. Dis. 2018, 120, 76-87. (284) Yang, H.-Y.; et al. Mol. Cell. Biol. 2003, 23 (20), 7096-7107. (285) Lee, M.-H.; et al. Semin. Cancer Biol. 2006, 16 (3), 225-234. (286) Wood, N. T.; et al. FEBS Lett. 2009, 583 (4), 615-620. (287) Okamoto, K.; et al. Mol. Cell. Biol. 2005, 25 (21), 9608-9620. (288) LeBron, C.; et al. EMBO J. 2006, 25 (6), 1196-1206. (289) Jin, Y.; et al. EMBO J. 2006, 25 (6), 1207-1218. (290) Lopez-Pajares, V.; et al. J. Biol. Chem. 2008, 283 (20), 13707-13713. (291) Lee, J.-H.; et al. J. Biol. Chem. 2011, 286 (7), 5136-5142. (292) Lee, J.-H.; et al. J. Biol. Chem. 2012, 287 (25), 20898-20903. (293) He, G.; et al. Mol. Cell. Biol. 2014, 34 (2), 148-157. (294) AMP-activated protein kinase induces p53 by phosphorylating MDMX and inhibiting its activity.—PubMed—NCBI https://www.ncbi.nlm.nih.gov/pubmed/24190973 (accessed Dec. 17, 2018). (295) Molzan, M.; et al. FEBS J. 2012, 279 (4), 563-571. (296) Molzan, M.; et al. Cell. Mol. Biol. Lett. 2013, 18 (1), 137-148. (297) Lim, R.; et al. J. Biol. Chem. 2002, 277 (43), 40997-41008. (298) Eilers, A. L.; et al. Mol. Cell. Biol. 2002, 22 (24), 8514-8526. (299) Merla, G.; et al. Hum. Mol. Genet. 2004, 13 (14), 1505-1514. (300) Li, M. V.; et al. Mol. Endocrinol. Baltim. Md 2008, 22 (7), 1658-1672. (301) Sato, S.; et al. J. Biol. Chem. 2016, 291 (20), 10515-10527. (302) Yang, A.-Q.; et al. Mol. Cell. Proteomics MCP 2017, 16 (7), 1233-1243. (303) Pallas, D. C.; et al. Science 1994, 265 (5171), 535-537. (304) Senften, M.; et al. J. Virol. 1997, 71 (9), 6990-6995. (305) Cullerd, X.; et al. J. Virol. 1998, 72 (1), 558-563. (306) Meili, R.; et al. Oncogene 1998, 16 (7), 903-907. (307) Zavrsnik, J.; et al. Oncotarget 2017, 8 (43), 73793-73809. (308) DNA Damage-Mediated c-Myc Degradation Requires 14-3-3 Sigma: Ingenta Connect https://www.ingentaconnect.com/content/asp/ch/2013/00000001/00000001/art00002 (accessed Dec. 10, 2018). (309) Phan, L.; et al. Nat. Commun. 2015, 6, 7530. (310) Yip, M. F.; et al. Cell Metab. 2008, 8 (5), 384-398. (311) Holman, G. D.; et al. Cell Metab. 2008, 8 (5), 344-346. (312) Dubois, F.; et al. Mol. Cell. Proteomics MCP 2009, 8 (11), 2487-2499. (313) Munnich, S.; et al. J. Mol. Biol. 2014, 426 (10), 2070-2081. (314) Toyo-oka, K.; et al. Nat. Genet. 2003, 34 (3), 274-285. (315) Taya, S.; et al. J. Neurosci. Off. J. Soc. Neurosci. 2007, 27 (1), 15-26. (316) Borisova, M. E.; et al. Nat. Commun. 2018, 9 (1), 1017. (317) Liao, W.; et al. FEBS J. 2005, 272 (8), 1845-1854. (318) Chhabra, S.; et al. Proc. Natl. Acad. Sci. U.S.A 2018, 115 (8), E1710-E1719. (319) Inglés-Esteve, J.; et al. PloS One 2012, 7 (5), e38347. (320) Zhou, X.-Y.; et al. Neurochem. Res. 2017, 42 (8), 2354-2362. (321) Lehoux, S.; et al. J. Biol. Chem. 2001, 276 (19), 15794-15800. (322) Amith, S. R.; et al. Oncotarget 2016, 7 (16), 21091-21113. (323) Amith, S. R.; et al. Cell. Signal. 2017, 29, 69-77. (324) Odunewu-Aderibigbe, A.; et al. Cell. Signal. 2017, 36, 145-153. (325) Ramakrishnan, G.; et al. Sci. Rep. 2015, 5, 8782. (326) Qin, S.; et al. Nat. Commun. 2014, 5, 3952. (327) Snow, A. J.; et al. Biol. Reprod. 2008, 79 (2), 337-347. (328) Rose, R.; et al. J. Struct. Biol. 2012, 180 (1), 65-72. (329) Tinti, M.; et al. Database J. Biol. Databases Curation 2014, 2014, bat085. (330) Fujita, N.; et al. J. Biol. Chem. 2002, 277 (32), 28706-28713. (331) Fujita, N.; Sato, S.; Tsuruo, T. J. Biol. Chem. 2003, 278 (49), 49254-49260. (332) Sekimoto, T.; Fukumoto, M.; Yoneda, Y. EMBO J. 2004, 23 (9), 1934-1942. (333) Yang, H.; et al. Oncogene 2006, 25 (33), 4585-4594. (334) Wang, Z.; et al. Mol. Cancer Ther. 2008, 7 (2), 341-349. (335) Short, J. D.; et al. Mol. Carcinog. 2010, 49 (5), 429-439. (336) Waterman, M. J.; et al. Nat. Genet. 1998, 19 (2), 175-178. (337) Rajagopalan, S.; et al. Nucleic Acids Res. 2008, 36 (18), 5983-5991. (338) Rajagopalan, S.; et al. Nucleic Acids Res. 2010, 38 (3), 893-906. (339) Schumacher, B.; et al. FEBS Lett. 2010, 584 (8), 1443-1448. (340) Doveston, R. G.; et al. FEBS Lett. 2017, 591 (16), 2449-2457. (341) Yang, T.; et al. Neuron 2012, 74 (1), 108-121. (342) Fonseca, B. D.; et al. J. Biol. Chem. 2007, 282 (34), 24514-24524. (343) Huang, B.; et al. Acta Pharmacol. Sin. 2005, 26 (10), 1253-1258. (344) Vander Haar, E.; et al. Nat. Cell Biol. 2007, 9 (3), 316-323. (345) Wang, L.; et al. J. Biol. Chem. 2008, 283 (23), 15619-15627. (346) Yu, F.; et al. J. Cereb. Blood Flow Metab. Off. J. Int. Soc. Cereb. Blood Flow Metab. 2008, 28 (1), 44-52. (347) Fonseca, B. D.; et al. Biochem. J. 2008, 411 (1), 141-149. (348) Nascimento, E. B. M.; et al. Cell. Signal. 2010, 22 (6), 961-967. (349) Hong-Brown, et al. J. Cell. Biochem. 2010, 109 (6), 1172-1184. (350) Shang, Y. C.; et al. Curr. Neurovasc. Res. 2012, 9 (4), 239-249. (351) Shin, M. J.; et al. Free Radic. Biol. Med. 2016, 97, 250-262. (352) Di Fede, G.; et al. J. Neuropathol. Exp. Neurol. 2007, 66 (2), 124-130. (353) Han, J.; et al. Int. J. Biochem. Cell Biol. 2014, 47, 20-28. (354) Jéru, I.; et al. Arthritis Rheum. 2005, 52 (6), 1848-1857. (355) Gao, W.; et al. Proc. Natl. Acad. Sci. U.S.A 2016, 113 (33), E4857-4866. (356) Masters, S. L.; et al. Sci. Transl. Med. 2016, 8 (332), 332ra45. (357) Park, Y. H.; et al. Nat. Immunol. 2016, 17 (8), 914-921. (358) Moghaddas, F.; et al. Ann. Rheum. Dis. 2017, 76 (12), 2085-2094. (359) Harapas, C. R.; et al. Mol. Cell Curr. Rheumatol. Rep. 2018, 20 (7), 40. (360) Gwinn, D. M.; et al. J. Mol. Cell 2008, 30 (2), 214-226. (361) Evren, S.; et al. J. Clin. Pathol. 2011, 64 (8), 683-688. (362) DeYoung, M. P.; et al. Genes Dev. 2008, 22 (2), 239-251. (363) Favier, F. B.; et al. Am. J. Physiol. Regul. Integr. Comp. Physiol. 2010, 298 (6), R1659-1666. (364) Britto, F. A.; et al. Am. J. Physiol. Endocrinol. Metab. 2014, 307 (11), E983-993. (365) Jin, H.-O.; et al. Oncogene 2011, 30 (35), 3792-3801. (366) Pieri, B. L. S.; et al. Horm. Metab. Res. Horm. Stoffwechselforschung Horm. Metab. 2014, 46 (9), 621-627. (367) Béguin, P.; et al. Biochem. J. 2005, 390 (Pt 1), 67-75. (368) Yang, C.-S.; et al. Cell Host Microbe 2012, 11 (3), 277-289. (369) Liu, H. M.; et al. Cell Host Microbe 2012, 11 (5), 528-537. (370) Chan, Y. K.; et al. Nat. Immunol. 2016, 17 (5), 523-530. (371) Gupta, S.; et al. PLoS Pathog. 2018, 14 (1), e1006852. (372) Gupta, S.; et al. PLoS Pathog. 2018, 14 (1), e1006852. (373) Riou, P.; et al. Cell 2013, 153 (3), 640-653. (374) Sluchanko, N. N.; et al. Biochem. Biophys. Res. Commun. 2017, 489 (4), 445-450. (375) Sluchanko, N. N.; et al. Sci. Rep. 2017, 7 (1), 12014. (376) Prokop, J. W.; et al. J. Am. Soc. Nephrol. JASN 2018, 29 (5), 1525-1535. (377) Lasserre, R.; et al. J. Cell Biol. 2011, 195 (5), 839-853. (378) Saha, M.; et al. A. Biochem. J. 2012, 447 (1), 159-166. (379) Ballone, A.; et al. J. Struct. Biol. 2018, 202 (3), 210-215. (380) Ballone, A.; et al. Protein X-Ray Crystallography of the 14-3-3ζ/SOS1 Complex. Data Brief 2018, 19, 1683-1687. https://doi.org/10.1016/j.dib.2018.06.060. (381) Jang, S.-W.; et al. J. Biol. Chem. 2009, 284 (36), 24512-24525. (382) Hong, Y.; et al. J. Biol. Chem. 2011, 286 (1), 777-786. (383) Faul, C.; et al. Nat. Med. 2008, 14 (9), 931-938. (384) Zhang, X.; et al. PloS One 2013, 8 (6), e67471. (385) Buvall, L.; et al. J. Am. Soc. Nephrol. JASN 2017, 28 (3), 837-851. (386) Kilisch, M.; et al. J. Cell Sci. 2016, 129 (4), 831-842. (387) Kilisch, M.; et al. Pflugers Arch. 2015, 467 (5), 1105-1120. (388) Rajan, S.; et al. J. Physiol. 2002, 545 (1), 13-26. (389) Zuzarte, M.; et al. J. Physiol. 2009, 587 (Pt 5), 929-952. (390) Anders, C.; et al. Chem. Biol. 2013, 20 (4), 583-593. (391) Sadik, G.; et al. Neurochem. 2009, 108 (1), 33-43. (392) Joo, Y.; et al. FASEB J. Off. Publ. Fed. Am. Soc. Exp. Biol. 2015, 29 (10), 4133-4144. (393) Liu, C.; et al. J. Biol. Chem. 2016, 291 (15), 8173-8188. (394) Qureshi, H. Y.; et al. Biochemistry 2013, 52 (37), 6445-6455. (395) Agarwal-Mawal, A.; et al. J. Biol. Chem. 2003, 278 (15), 12722-12728. (396) Hashiguchi, M.; et al. J. Biol. Chem. 2000, 275 (33), 25247-25254. (397) Hernandez, F.; et al. J. Neurosci. Lett. 2004, 357 (2), 143-146. (398) Kumar, P.; et al. J. Alzheimers Dis. JAD 2015, 43 (2), 341-361. (399) Li, T.; Paudel, H. K. PloS One 2016, 11 (8), e0160635. (400) Li, T.; Paudel, H. K. Neurosci. Lett. 2007, 414 (3), 203-208. (401) Papanikolopoulou, K.; et al. Hum. Mol. Genet. 2018, 27 (13), 2244-2261. (402) Sluchanko, N. N.; et al. Biochem. Biophys. Res. Commun. 2009, 379 (4), 990-994. (403) Stefanoska, K.; et al. J. Biol. Chem. 2018, 293 (10), 3710-3719. (404) Sugimori, K.; et al. Psychiatry Clin. Neurosci. 2007, 61 (2), 159-167. (405) Tugaeva, K. V.; et al. PloS One 2017, 12 (6), e0178933. (406) Yuan, Z.; et al. J. Biol. Chem. 2004, 279 (25), 26105-26114. (407) Frøsig, C.; et al. J. Physiol. 2010, 588 (Pt 22), 4539-4548. (408) Mafakheri, S.; et al. J. Biol. Chem. 2018, 293 (46), 17853-17862. (409) Madigan, J. P.; et al. J. Biol. Chem. 2018, 293 (42), 16142-16159. (410) YWHA/14-3-3 proteins recognize phosphorylated TFEB by a noncanonical mode for controlling TFEB cytoplasmic localization.—PubMed—NCBI https://www.ncbi.nlm.nih.gov/pubmed/?term=30653408 (accessed Apr. 27, 2019). (411) Seimiya, H.; et al. EMBO J. 2000, 19 (11), 2652-2661. (412) Zhang, P.; et al. FASEB J. Off. Publ. Fed. Am. Soc. Exp. Biol. 2003, 17 (6), 767-769. (413) Xi, P.; et al. J. Cell. Biochem. 2013, 114 (2), 409-417. (414) Arancio, W.; et al. Rejuvenation Res. 2014, 17 (2), 161-167. (415) Chen, H.; et al. Cell Res. 2019. (416) Wu, D.; et al. Nature 2018, 559 (7715), 637-641. (417) Mishra, S.; et al. J. Biochem. Biophys. Res. Commun. 2006, 347 (4), 1166-1170. (418) Quinn, B. R.; et al. J. Neurosci. Res. 2018, 96 (7), 1150-1158. (419) Kim, S.-Y. Med. Sci. Basel Switz. 2018, 6 (4). (420) Ghorbani, S.; et al. Amino Acids 2016, 48 (5), 1221-1229. (421) Hritz, J.; et al. Biophys. J. 2014, 107 (9), 2185-2194. (422) Kleppe, R.; et al. Mol. Cell. Proteomics MCP 2014, 13 (8), 2017-2030. (423) Walsen, K.; et al. J. Expert Opin. Ther. Targets 2017, 21 (2), 167-180. (424) Nellist, M.; et al. J. Biol. Chem. 2002, 277 (42), 39417-39424. (425) Li, Y.; Inoki, K.; Yeung, R.; Guan, K.-L. J. Biol. Chem. 2002, 277 (47), 44593-44596. (426) Liu, M. Y.; et al. Cancer Res. 2002, 62 (22), 6475-6480. (427) Shumway, S. D.; et al. J. Biol. Chem. 2003, 278 (4), 2089-2092. (428) Li, Y.; et al. J. Biol. Chem. 2003, 278 (16), 13663-13671. (429) Reincke, M.; et al. Nat. Genet. 2015, 47 (1), 31-38. (430) Kino, T.; et al. DNA Cell Biol. 2004, 23 (4), 193-205. (431) Kino, T.; et al. Acad. Sci. 2004, 1024, 153-167. (432) Kino, T.; et al. Diabetes 2005, 54 (1), 23-31. (433) Kino, T.; et al. J. Virol. 2005, 79 (5), 2780-2787. (434) Matsuda, N.; et al. Microbes Infect. 2006, 8 (12-13), 2736-2744. (435) Huard, S.; et al. J. Virol. 2008, 82 (6), 2904-2917. (436) Bolton, D. L.; et al. Biol. Direct 2008, 3, 17. (437) Lam, T.; et al. PloS One 2013, 8 (11), e80414. (438) Vassilev, A.; et al. Genes Dev. 2001, 15 (10), 1229-1241. (439) Basu, S.; et al. Mol. Cell 2003, 11 (1), 11-23. (440) Zhao, B.; et al. Genes Dev. 2007, 21 (21), 2747-2761. (441) Hong, J.-H.; et al. Science 2005, 309 (5737), 1074-1078. (442) Kanai, F.; et al. EMBO J. 2000, 19 (24), 6778-6791. (443) Wang, K.; et al. Biochem. Cell Biol. Biochim. Biol. Cell. 2009, 87 (1), 77-91. (444) Zhao, B.; et al. Genes Dev. 2010, 24 (1), 72-85. (445) Schlegelmilch, K.; et al. Cell 2011, 144 (5), 782-795. (446) Schumacher, B.; et al. Acta Crystallograph. Sect. F Struct. Biol. Cryst. Commun. 2010, 66 (Pt 9), 978-984. (447) Lei, Q.-Y.; et al. Mol. Cell. Biol. 2008, 28 (7), 2426-2436. (448) Liu, C.; et al. Int. J. Biochem. Mol. Biol. 2011, 2 (3), 247-256. (449) Chan, S. W.; et al. Cancer Res. 2008, 68 (8), 2592-2598. (450) Chen, Q.; et al. Genes Dev. 2015, 29 (12), 1285-1297. (451) Hong, A. W.; et al. EMBO Rep. 2017, 18 (1), 72-86. (452) Moon, S.; et al. EMBO Rep. 2017, 18 (1), 61-71. (453) Sambandam, S. A. T.; et al. Invest. Dermatol. 2015, 135 (6), 1621-1628. (454) Xu, G.; et al. Oncotarget 2016, 7 (32), 52150-52165. (455) Tanas, M. R.; et al. Oncogene 2016, 35 (7), 929-938. (456) Kofler, M.; et al. Nat. Commun. 2018, 9 (1), 4966. (457) Ramser, E. M.; et al. Mol. Cell. Neurosci. 2010, 45 (1), 66-74. (458) Wang, B.; et al. J. Neurosci. Off. J. Soc. Neurosci. 2018. (459) Wakabayashi, K.; et al. Neurosci. Lett. 2018, 674, 171-175. (460) Plotegher, N.; et al. Hum. Mol. Genet. 2014, 23 (21), 5615-5629. (461) Yacoubian, T. A.; et al. Cell Death Dis. 2010, 1, e2. (462) Shirakashi, Y.; et al. Acta Neuropathol. (Berl.) 2006, 112 (6), 681-689. (463) Ostrerova, N.; et al. Neurosci. Off. J. Soc. Neurosci. 1999, 19 (14), 5782-5791. (464) Kawamoto, Y.; et al. J. Neuropathol. Exp. Neurol. 2002, 61 (3), 245-253. (465) Berg, T. Angew. Chem. Int. Ed Engl. 42, 2462-81 (2003). (466) Wells, J. A. & McClendon, C. L. Nature 450, 1001-9 (2007). (467) Arkin, M. R., et al. Chem. Biol. 21, 1102-1114 (2014). (468) Scott, D. E., et al. J. Nat. Rev. Drug Discov. 15, 533-550 (2016). (469) Fischer, G., et al. Curr. Opin. Biotechnol. 35, 78-85 (2015). (470) Andrei, S. A., et al. Expert Opin. Drug Discov. 12, 925-940 (2017). (471) Zarzycka, B., et al. Drug Discov. Today 21, 48-57 (2016). (472) Waring, M. J., et al. Nat. Chem. Biol. 12, 1097-1104 (2016). (473) Bulawa, C. E., et al. R. Proc. Natl. Acad. Sci. U.S.A. 109, 9629-9634 (2012). (474) Hughes, S. J. & Ciulli, A. Essays Biochem. 61, 505-516 (2017). (475) Erlanson, D. A., et al. Proc. Natl. Acad. Sci. U.S.A 97, 9367-9372 (2000). (476) Ostrem, J. M., et al. Nature 503, 548-51 (2013). (477) Erlanson, D. A., et al. Bioorg. Med. Chem. Lett. 21, 3078-3083 (2011). (478) Hyde, J., et al. Biochemistry 42, 6475-6483 (2003). (479) Raimundo, B. C., et al. J. Med. Chem. 47, 3111-3130 (2004). (480) Molzan, M. & Ottmann, C. J. Mol. Biol. 423, 486-495 (2012). (481) Sluchanko, N. N., et al. Struct. Lond. Engl. 1993 25, 305-316 (2017). (482) De Vries-van Leeuwen, I. J., et al. Proc. Natl. Acad. Sci. U.S.A 110, 8894-9 (2013). (483) Schumacher, B., et al. FEBS Lett. 584, 1443-1448 (2010). (484) Zhao, J., et al. Semin. Cell Dev. Biol. 22, 705-712 (2011). (485) Aghazadeh, Y. & Papadopoulos, V. T. Drug Discov. Today 21, 278-287 (2016). (486) Stevers, L. M., et al. J. Med. Chem. 61, 3755-3778 (2018). (487) Erlanson, D. A., et al. Annu. Rev. Biophys. Biomol. Struct. 33, 199-223 (2004). (488) Pennington, K. L., et al. Oncogene 37, 5587 (2018). (489) Zhao, J., et al. Proc. Natl. Acad. Sci. U.S.A 108, 16212-16216 (2011). (490) Milroy, L.-G., et al. Angew. Chem. Int. Ed. 54, 15720-15724 (2015). (491) Andrei, S. A., et al. ACS Chem. Neurosci. 9, 2639-2654 (2018). (492) Hallenbeck, K. K., et al. SLAS Discov. 23, 183-192 (2018). (493) Ottmann, C., et al. EMBO J. 26, 902-913 (2007). (494) Sijbesma, E., et al. Biochemistry 56, 3972-3982 (2017). (495) Anders, C., et al. Chem. Biol. 20, 583-593 (2013). (496) Burlingame, M. A., et al. ACS Comb. Sci. 13, 205-208 (2011). (497) Turner, D. M., et al. ACS Comb. Sci. 16, 661-664 (2014). (498) Arkin, M. R.; et al. Chem. Biol. 2014, 21 (9), 1102-1114. (499) Bier, D.; et al. Prog. Biophys. Mol. Biol. 2015, 119 (1), 10-19. (500) Andrei, S. A.; et al. Expert Opin. Drug Discov. 2017, 12 (9), 925-940. (501) Price, A. J.; et al. 2017, 61 (5), 475-484. (502) Erlanson, D. A.; et al. Proc. Natl. Acad. Sci. U.S.A 2000, 97 (17), 9367-9372. (503) Sijbesma, E.; et al. J. Am. Chem. Soc. 2019. (504) Johnson, C.; et al. Biochem. J. 2010, 427 (1), 69-78. (505) de Vink, P. J.; et al. Chem. Sci. 2019, 10 (10), 2869-2874. (506) Ottmann, C.; et al. Mol. Cell 2007, 25 (3), 427-440. (507) Sharff, A., et al. Pipedream, version 1.2.1 (Global Phasing Ltd, Cambridge, United Kingdom) (2011). (508) Vonrhein, C., et al. Acta Crystallogr. D Biol. Crystallogr. 67, 293-302 (2011). (509) Kabsch, W. XDS. Acta Crystallogr. D Biol. Crystallogr. 66, 125-132 (2010). (510) Winn, M. D., et al. Acta Crystallogr. D Biol. Crystallogr. 67, 235-242 (2011). (511) Evans, P. R. et al. Acta Crystallogr. D Biol. Crystallogr. 69, 1204-1214 (2013). (512) McCoy, A. J., et al. J. Appl. Crystallogr. 40, 658-674 (2007). (513) Bricogne, G., et al. BUSTER. (Global Phasing Ltd, Cambridge, United Kingdom) (2011). (514) Emsley, P. et al. Acta Crystallogr. D Biol. Crystallogr. 60, 2126-2132 (2004). (515) Moriarty, N. W., et al. Acta Crystallogr. D Biol. Crystallogr. 65, 1074-1080 (2009). (516) Adams, P. D., et al. Acta Crystallogr. D Biol. Crystallogr. 66, 213-221 (2010). (517) Afonine, P. V., et al. Acta Crystallogr. D Biol. Crystallogr. 68, 352-367 (2012). (518) Gossert, A. D., et al. J. Biomol. NMR 43, 211-217 (2009). (519) Hajduk, P. J., et al. Science 278, 497-499 (1997). (520) Karplus, P. A. et al. Science 336, 1030-1033 (2012).

What is claimed is:

1. A method of identifying a chemical compound that modulates the binding of a protein to a client protein, the method comprising:
contacting a first candidate compound with a protein comprising a solvent exposed reactive amino acid side chain proximal to a client protein binding site, thereby forming a protein conjugate, wherein said first candidate compound comprises a first candidate chemical moiety covalently bound to a first reactive group, wherein said first reactive group is specifically reactive with said solvent exposed reactive amino acid side chain, which is not a cysteine side chain;
contacting said protein conjugate with said client protein thereby forming a conjugate-client complex; and
detecting a change in stability of said conjugate-client complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

2. The method of claim 1, wherein the method identifies a chemical compound that stabilizes the binding of a protein to a client protein comprising detecting an increase in stability of said conjugate-client complex relative to the stability of a protein-client complex.

3. The method of claim 1, wherein the protein is a 14-3-3 protein.

4. The method of claim 3, wherein the solvent exposed reactive amino acid side chain of the 14-3-3 protein, proximal to the 14-3-3 client protein binding site, is the side chain of a methionine, tryptophan, tyrosine, lysine or histidine.

5. The method of claim 3, wherein the 14-3-3 protein comprises an amino acid mutation.

6. The method of claim 3, wherein the 14-3-3 client protein is ERα, ERRγ, TASK3, ExoS, MYC, Rel A, FOXO-1, p65, or TAZ.

7. The method of claim 1, wherein the conjugate-client complex further comprises a second candidate compound covalently bound to said first candidate compound.

8. The method of claim 1, wherein the conjugate-client complex is further contacted with a second candidate compound, such that the conjugate-client complex is non-covalently attached to said second candidate compound.

9. A method of identifying a chemical compound that modulates binding of a protein to a client protein, the method comprising:
contacting a client protein with a protein comprising a solvent exposed reactive amino acid side chain proximal to a client protein binding site, thereby forming a protein-client complex;
contacting said protein-client complex with a first candidate compound thereby forming a conjugate-client complex, wherein said first candidate compound comprises a first candidate chemical moiety covalently bound to a first reactive group, wherein said first reactive group is specifically reactive with said solvent exposed reactive amino acid side chain, which is not a cysteine side chain, and wherein said first candidate compound covalently attaches to said solvent exposed reactive amino acid side chain to form said conjugate-client complex; and
detecting a change in stability of said conjugate-client complex relative to the stability of said protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

10. The method of claim 9, wherein the method identifies a chemical compound that stabilizes the binding of a protein to a client protein comprising detecting an increase in stability of said conjugate-client complex relative to the stability of a protein-client complex.

11. The method of claim 9, wherein the protein is a 14-3-3 protein.

12. The method of claim 11, wherein the solvent exposed reactive amino acid side chain of the 14-3-3 protein, proximal to the 14-3-3 client protein binding site, is the side chain of a methionine, tryptophan, tyrosine, lysine or histidine.

13. The method of claim 11, wherein the 14-3-3 protein comprises an amino acid mutation.

14. The method of claim 11, wherein the 14-3-3 client protein is ERα, ERRγ, TASK3, ExoS, MYC, Rel A, FOXO-1, p65, or TAZ.

15. The method of claim 9, wherein the conjugate-client complex further comprises a second candidate compound covalently bound to said first candidate compound.

16. The method of claim 9, wherein the conjugate-client complex is further contacted with a second candidate compound, such that the conjugate-client complex is non-covalently attached to said second candidate compound.

17. A method of identifying a chemical compound that modulates binding of a protein to a client protein, the method comprising:
contacting a first candidate compound with a client protein comprising a solvent exposed reactive amino acid side chain, thereby forming a client protein conjugate, wherein said first candidate compound comprises a first candidate chemical moiety covalently bound to a first reactive group, wherein said first reactive group is specifically reactive with said solvent exposed reactive amino acid side chain;
contacting said client protein conjugate with a protein thereby forming a conjugate-protein complex; and
detecting a change in stability of said conjugate-protein complex relative to the stability of a protein-client complex, wherein said protein-client complex comprises said client protein and said protein in the absence of said first candidate compound covalently bound to said solvent exposed reactive amino acid side chain, thereby identifying said first candidate compound as the first chemical compound that modulates binding of said protein to said client protein.

18. The method of claim 17, wherein the method identifies a chemical compound that stabilizes the binding of a protein to a client protein comprising detecting an increase in stability of said conjugate-protein complex relative to the stability of a protein-client complex.

19. The method of claim 17, wherein the protein is a 14-3-3 protein.

20. The method of claim 19, wherein the solvent exposed reactive amino acid side chain of the 14-3-3 client protein is the side chain of a cysteine, methionine, tryptophan, tyrosine, lysine or histidine.

21. The method of claim 20, wherein the solvent exposed reactive amino acid side chain of the 14-3-3 client protein is the side chain of a cysteine.

22. The method of claim 21, wherein the solvent exposed reactive amino acid side chain of the 14-3-3 client protein comprises a thiol.

23. The method of claim 19, wherein the 14-3-3 client protein comprises an amino acid mutation.

24. The method of claim 19, wherein the 14-3-3 client protein is ERα, ERRγ, TASK3, ExoS, MYC, Rel A, FOXO-1, p65, or TAZ.

25. The method of claim 17, wherein the conjugate-protein complex further comprises a second candidate compound covalently bound to said first candidate compound.

26. The method of claim 17, wherein the conjugate-protein complex is further contacted with a second candidate compound, such that the conjugate-protein complex is non-covalently attached to said second candidate compound.

27. A method of identifying a chemical compound that modulates binding of a protein to a client protein, the method comprising:
contacting a protein with a client protein comprising a solvent exposed reactive amino acid side chain thereby forming a protein-client complex;
contacting said protein-client complex with a first candidate compound thereby forming a conjugate-protein complex, wherein said first candidate compound comprises a first candidate chemical moiety covalently bound to a first reactive group, wherein said first reactive group is specifically reactive with said solvent exposed reactive amino acid side chain, and wherein said first candidate compound covalently attaches to said solvent exposed reactive amino acid side chain to form said conjugate-protein complex; and
detecting a change in stability of said conjugate-protein complex relative to the stability of said protein-client complex, wherein said protein-client complex comprises said protein and said client protein in the absence of said first candidate compound covalently bound to said solvent exposed reactive amino acid side chain, thereby identifying said first candidate compound as the first chemical compound that modulates binding of said protein to said client protein.

28. The method of claim 27, wherein the method identifies a chemical compound that stabilizes the binding of a protein to a client protein comprising detecting an increase in stability of said conjugate-protein complex relative to the stability of a protein-client complex.

29. The method of claim 27, wherein the protein is a 14-3-3 protein.

30. The method of claim 29, wherein the solvent exposed reactive amino acid side chain of the 14-3-3 client protein is the side chain of a cysteine, methionine, tryptophan, tyrosine, lysine or histidine.

31. The method of claim 30, wherein the solvent exposed reactive amino acid side chain of the 14-3-3 client protein is the side chain of a cysteine.

32. The method of claim 31, wherein the solvent exposed reactive amino acid side chain of the 14-3-3 client protein comprises a thiol.

33. The method of claim 29, wherein the 14-3-3 client protein comprises an amino acid mutation.

34. The method of claim 29, wherein the 14-3-3 client protein is ERα, ERRγ, TASK3, ExoS, MYC, Rel A, FOXO-1, p65, or TAZ.

35. The method of claim 27, wherein the conjugate-protein complex further comprises a second candidate compound covalently bound to said first candidate compound.

36. The method of claim 27, wherein the conjugate-protein complex is further contacted with a second candidate compound, such that the conjugate-protein complex is non-covalently attached to said second candidate compound.

37. A method of treating a disease in a subject in need thereof, the method comprising administering to the subject an effective amount of a chemical compound that stabilizes binding of a protein to a client protein, wherein the chemical compound is identified by a method of one of claims 1 to 36.

38. The method of claim 37, wherein the disease is cancer, inflammatory disease, metabolic disease, neurodegenerative disease, or infection.

39. A compound having the general formula:
R¹-L¹-W-L³-R³,
wherein:
L¹ and L³ are independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
W is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is -L^{3A}-L^{3B}-E3, hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{3A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —NH—, —C(O)NH—, —NHC(O)NH—, substituted or unsubstituted heteroalkylene, substituted or unsubstituted heterocycloalkylene, or substituted or unsubstituted heteroarylene;
E3 is —SH, 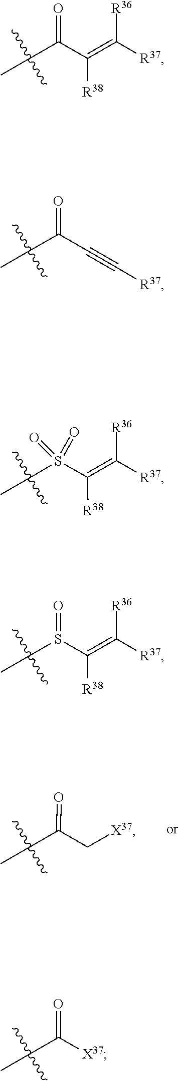
R³⁶, R³⁷, and R³⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹, X³, and X³⁷ are independently —F, —Cl, —Br, or —I;
n1 and n3 are independently an integer from 0 to 4; and
m1, v1, m3, and v3 are independently 1 or 2.

40. The compound of claim 39, wherein
R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to membered heteroaryl.

41. The compound of claim 39, wherein
R¹ is 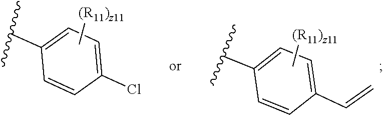
R¹¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹¹ substituents may optionally be joined to form a substituted or unsubstituted cycloalkyl, substituted or unsubstituted heterocycloalkyl, substituted or unsubstituted aryl, or substituted or unsubstituted heteroaryl; and
z11 is an integer from 0 to 4.

42. The compound of claim 39, wherein
R¹ is

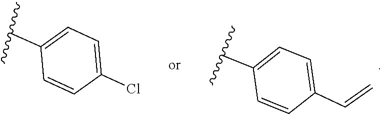

43. The compound of claim 39, further comprising R², wherein
R² is a 14-3-3 C38 binding moiety.

44. The compound of claim 43, wherein R² is a 14-3-3 C38 non-covalent binding moiety.

45. The compound of claim 43, wherein R² is a 14-3-3 C38 covalent binding moiety.

46. The compound of claim 43, wherein
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl;
X² is —F, —Cl, —Br, or —I;
n2 is an integer from 0 to 4; and
m2 and v2 are independently 1 or 2.

47. The compound of claim 43, wherein
R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to membered heteroaryl.

48. The compound of claim 43, wherein
R² is -L^{2A}-L^{2B}-E2;
L^{2A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{2B} is a bond, —NH—, —C(O)NH—, —NHC(O)NH—, substituted or unsubstituted heteroalkylene, substituted or unsubstituted heterocycloalkylene, or substituted or unsubstituted heteroarylene;
E2 is —SH, —SSR²⁶, 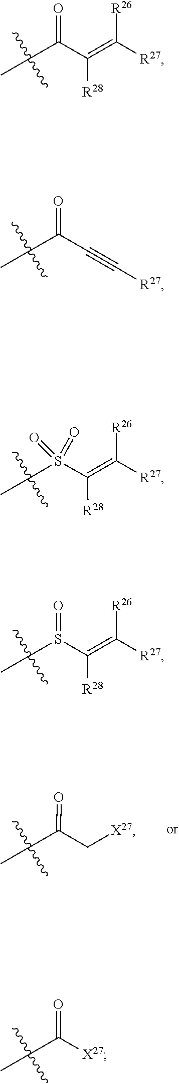
R²⁶, R²⁷, and R²⁸ are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X²⁷ is —F, —Cl, —Br, or —I.

49. The compound of claim 43, wherein
R² is -L^{2A}-L^{2B}-E2;
L^{2A} is a bond;
L^{2B} is —NH—; and
E2 is

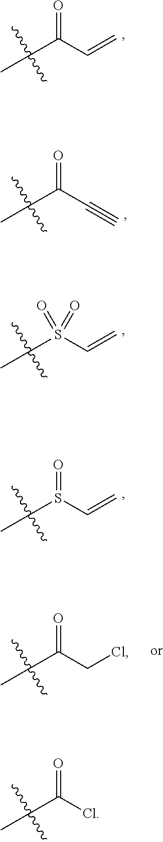

50. The compound of claim 39, wherein W is substituted with -L⁵-R⁵, wherein
L⁵ is a substituted or unsubstituted covalent linker; and
R⁵ is a 14-3-3 D215 binding moiety.

51. The compound of claim 50, wherein
R⁵ is hydrogen, halogen, —CX⁵₃, —CHX⁵₂, —CH₂X⁵, —OCX⁵₃, —OCH₂X⁵, —OCHX⁵₂, —CN, —SOᵥ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X⁵ is —F, —Cl, —Br, or —I;
n5 is an integer from 0 to 4; and
m5 and v5 are independently 1 or 2.

52. The compound of claim 50, wherein
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N3, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

53. The compound of claim 39, wherein
R³ is -L^{3A}-L^{3B}-E3;
L^{3A} is a bond;
μL^{3B} is —NH—; and
E3 is

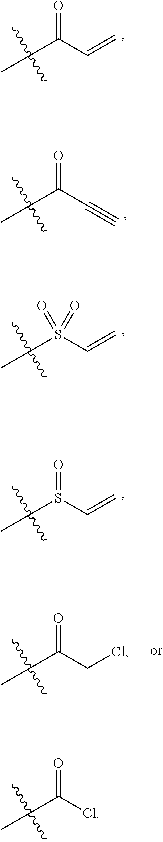

54. A compound having the general formula:
R²-L²-W-L³-R³
wherein:
L² and L³ are independently a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR²CNR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
W is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R³ is -L^{3A}-L^{3B}-E3, hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —C(NR^{3C})NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{3A} is a bond, —S(O)₂—, —NH—, —O—, —S—, —C(O)—, —NHS(O)₂—, —S(O)₂NH—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —NH—, —C(O)NH—, —NHC(O)NH—, substituted or unsubstituted heteroalkylene, substituted or unsubstituted heterocycloalkylene, or substituted or unsubstituted heteroarylene;
E3 is —SH, 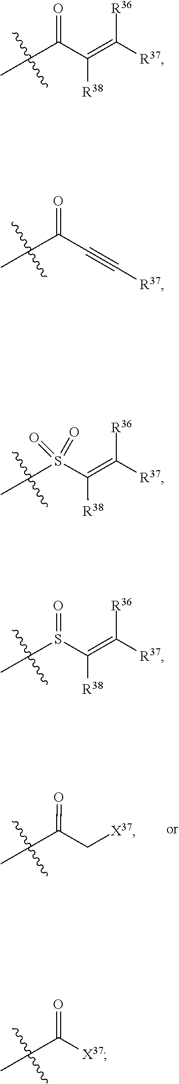
R³⁶, R³⁷, and R³⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X², X³, and X³⁷ are independently —F, —Cl, —Br, or —I;
n2 and n3 are independently an integer from 0 to 4; and
m2, v2, m3, and v3 are independently 1 or 2.

55. The compound of claim 54, further comprising R¹, wherein
R¹ is a 14-3-3 K120 binding moiety.

56. The compound of claim 55, wherein R¹ is a 14-3-3 K120 covalent binding moiety.

57. The compound of claim 55, wherein R¹ is a 14-3-3 K120 non-covalent binding moiety.

58. The compound of claim 55, wherein
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is —F, —Cl, —Br, or —I;
n1 is an integer from 0 to 4; and
m1 and v1 are independently 1 or 2.

59. The compound of claim 55, wherein
R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to membered heteroaryl.

60. The compound of claim 55, wherein
R¹ is 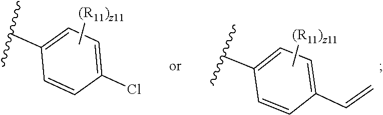
R¹¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹¹ substituents may optionally be joined to form a substituted or unsubstituted cycloalkyl, substituted or unsubstituted heterocycloalkyl, substituted or unsubstituted aryl, or substituted or unsubstituted heteroaryl; and
z11 is an integer from 0 to 4.

61. The compound of claim 55, wherein
R¹ is

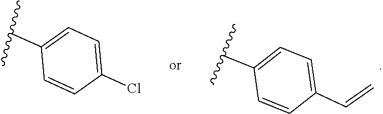

62. The compound of claim 54, wherein
R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to membered heteroaryl.

63. The compound of claim 54, wherein W is substituted with -L⁵-R⁵, wherein
L⁵ is a substituted or unsubstituted covalent linker; and
R⁵ is a 14-3-3 D215 binding moiety.

64. The compound of claim 63, wherein
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, —C(NR^{5C})NR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X⁵ is —F, —Cl, —Br, or —I;
n5 is an integer from 0 to 4; and
m5 and v5 are independently 1 or 2.

65. The compound of claim 63, wherein
R⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, —C(NH)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

66. The compound of claim 54, wherein
R³ is -L^{3A}-L^{3B}-E3;
L^{3A} is a bond;
L^{3B} is —NH—; and
E3 is

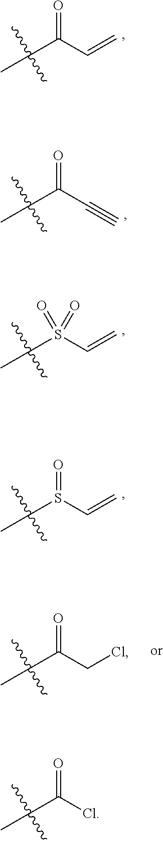

67. A pharmaceutical composition comprising the compound of any one of claims 39 to 66, or a pharmaceutically acceptable salt thereof, and a pharmaceutically acceptable excipient.

68. A method of increasing the level of a 14-3-3 protein-client protein complex in a subject, said method comprising administering a compound of one of claims 39 to 66 to said subject.

69. The method of claim 68, wherein the client protein of the 14-3-3 protein-client protein complex is an estrogen receptor.

70. The method of claim 68, wherein the client protein of the 14-3-3 protein-client protein complex is TAZ.

71. The method of claim 68, wherein the client protein of the 14-3-3 protein-client protein complex is p65.

72. A method of increasing the level of a 14-3-3 protein-client protein complex in a cell, said method comprising contacting the cell with a compound of one of claims 39 to 66.

73. A method of treating an inflammatory disease, cancer, an autoimmune disease, a neurodegenerative disease, a metabolic disease, or cystic fibrosis in a subject in need thereof, said method comprising administering to the subject in need thereof an effective amount of a compound of one of claims 39 to 66.

74. A method of treating a cancer in a subject in need thereof, said method comprising administering to the subject in need thereof an effective amount of a compound of one of claims 39 to 66.

75. The method of claim 74, wherein the cancer is breast cancer.

76. The method of claim 74, further comprising co-administering an anti-cancer agent to said subject in need.


SEQUENCE LISTING (57 sequences)

SEQ ID NO: 1 - protein, 19 aa, Artificial Sequence
  Synthetic polypeptide
  MOD_RES (10)..(10): PHOSPHORYLATION
SRSGLYRSPSMPENLNRPR

SEQ ID NO: 2 - protein, 15 aa, Artificial Sequence
  Synthetic polypeptide
  MISC_FEATURE (14)..(14): Phosphorylated Thr-594
KYYITGEAEGFPATV

SEQ ID NO: 3 - protein, 15 aa, Artificial Sequence
  Synthetic polypeptide
  MOD_RES (1)..(1): ACETYLATION
  MOD_RES (14)..(14): PHOSPHORYLATION
KYYITGEAEGFPATV

SEQ ID NO: 4 - protein, 5 aa, Artificial Sequence
  Synthetic polypeptide
  MOD_RES (1)..(1): ACETYLATION
  MOD_RES (4)..(4): PHOSPHORYLATION
RRKSV

SEQ ID NO: 5 - protein, 13 aa, Artificial Sequence
  Synthetic polypeptide
  MOD_RES (1)..(1): ACETYLATION
  MOD_RES (4)..(4): PHOSPHORYLATION
  MOD_RES (13)..(13): AMIDATION
RSHSSPASLQLGT

SEQ ID NO: 6 - protein, 15 aa, Artificial Sequence
  Synthetic polypeptide
  MOD_RES (1)..(1): ACETYLATION
  MOD_RES (15)..(15): AMIDATION
SGHGQGLLDALDLAS

SEQ ID NO: 7 - protein, 8 aa, Artificial Sequence
  Synthetic polypeptide
  MISC_FEATURE (1)..(1): 5-FAM-labeled
  MOD_RES (7)..(7): PHOSPHORYLATION
AEGFPATV

SEQ ID NO: 8 - protein, 6 aa, Artificial Sequence
  Synthetic polypeptide
IPGRRS

SEQ ID NO: 9 - protein, 13 aa, Artificial Sequence
  Synthetic polypeptide
  MISC_FEATURE (7)..(7): Phosphorylated Ser-45
EGRSAGSIPGRRS

SEQ ID NO: 10 - protein, 33 aa, Artificial Sequence
  Synthetic polypeptide
  MISC_FEATURE (7)..(7): Phosphorylated Ser-45
  MOD_RES (27)..(27): PHOSPHORYLATION
EGRSAGSIPGRRSGSGGGSGPSDRELSEPMEFQ

SEQ ID NO: 11 - protein, 10 aa, Artificial Sequence
  Synthetic polypeptide
  MISC_FEATURE (4)..(4): Phosphorylated Ser-89
RSHSSPASLQ

SEQ ID NO: 12 - protein, 32 aa, Artificial Sequence
  Synthetic polypeptide
  MISC_FEATURE (26)..(26): Phosphorylated Thr-387
SRAHSSHLKSKKGQSTSRHKKLMFKTEGPDSD

SEQ ID NO: 13 - protein, 8 aa, Artificial Sequence
  Synthetic polypeptide
  MISC_FEATURE (7)..(7): Phosphorylated Thr-594
AEGFPATV

SEQ ID NO: 14 - protein, 8 aa, Artificial Sequence
  Synthetic polypeptide
  MOD_RES (7)..(7): PHOSPHORYLATION
AEGFPATV

SEQ ID NO: 15 - protein, 11 aa, Artificial Sequence
  Synthetic polypeptide
EKRMSRSSGRV

SEQ ID NO: 16 - protein, 11 aa, Artificial Sequence
  Synthetic polypeptide
KRMSRSSGRVY

SEQ ID NO: 17 - protein, 11 aa, Artificial Sequence
  Synthetic polypeptide
YYFNHITNASQ

SEQ ID NO: 18 - protein, 11 aa, Artificial Sequence
  Synthetic polypeptide
NHITNASQWER

SEQ ID NO: 19 - protein, 11 aa, Artificial Sequence
  Synthetic polypeptide
SQWERPSGNSS

SEQ ID NO: 20 - protein, 11 aa, Artificial Sequence
  Synthetic polypeptide
ERPSGNSSSGG

SEQ ID NO: 21 - protein, 11 aa, Artificial Sequence
  Synthetic polypeptide
RPSGNSSSGGK

SEQ ID NO: 22 - protein, 11 aa, Artificial Sequence
  Synthetic polypeptide
PSGNSSSGGKN

SEQ ID NO: 23 - protein, 11 aa, Artificial Sequence
  Synthetic polypeptide
PARVRCSHLLV

SEQ ID NO: 24 - protein, 11 aa, Artificial Sequence
  Synthetic polypeptide
HLLVKHSQSRR

SEQ ID NO: 25 - protein, 11 aa, Artificial Sequence
  Synthetic polypeptide
LVKHSQSRRPS

SEQ ID NO: 26 - protein, 11 aa, Artificial Sequence
  Synthetic polypeptide
SQSRRPSSWRQ

SEQ ID NO: 27 - protein, 11 aa, Artificial Sequence
  Synthetic polypeptide
QSRRPSSWRQE

SEQ ID NO: 28 - protein, 11 aa, Artificial Sequence
  Synthetic polypeptide
WRQEKITRTKE

SEQ ID NO: 29 - protein, 11 aa, Artificial Sequence
  Synthetic polypeptide
QEKITRTKEEA

SEQ ID NO: 30 - protein, 11 aa, Artificial Sequence
  Synthetic polypeptide
YIQKIKSGEED

SEQ ID NO: 31 - protein, 11 aa, Artificial Sequence
  Synthetic polypeptide
GEEDFESLASQ

SEQ ID NO: 32 - protein, 11 aa, Artificial Sequence
  Synthetic polypeptide
DFESLASQFSD

SEQ ID NO: 33 - protein, 11 aa, Artificial Sequence
  Synthetic polypeptide
SLASQFSDCSS

SEQ ID NO: 34 - protein, 11 aa, Artificial Sequence
  Synthetic polypeptide
SQFSDCSSAKA

SEQ ID NO: 35 - protein, 11 aa, Artificial Sequence
  Synthetic polypeptide
QFSDCSSAKAR

SEQ ID NO: 36 - protein, 11 aa, Artificial Sequence
  Synthetic polypeptide
GDLGAFSRGQM

SEQ ID NO: 37 - protein, 11 aa, Artificial Sequence
  Synthetic polypeptide
KPFEDASFALR

SEQ ID NO: 38 - protein, 11 aa, Artificial Sequence
  Synthetic polypeptide
ASFALRTGEMS

SEQ ID NO: 39 - protein, 11 aa, Artificial Sequence
  Synthetic polypeptide
LRTGEMSGPVF

SEQ ID NO: 40 - protein, 11 aa, Artificial Sequence
  Synthetic polypeptide
MSGPVFTDSGI

SEQ ID NO: 41 - protein, 11 aa, Artificial Sequence
  Synthetic polypeptide
GPVFTDSGIHI

SEQ ID NO: 42 - protein, 8 aa, Artificial Sequence
  Synthetic polypeptide
IHIILRTE

SEQ ID NO: 43 - protein, 17 aa, Artificial Sequence
  Synthetic polypeptide
  MOD_RES (11)..(11): PHOSPHORYLATION
LVKHSQSRRPSSWRQEK

SEQ ID NO: 44 - protein, 17 aa, Artificial Sequence
  Synthetic polypeptide
  MOD_RES (12)..(12): PHOSPHORYLATION
LVKHSQSRRPSSWRQEK

SEQ ID NO: 45 - protein, 33 aa, Artificial Sequence; identical to SEQ ID NO: 10
  Synthetic polypeptide
  MOD_RES (7)..(7): PHOSPHORYLATION
  MOD_RES (27)..(27): PHOSPHORYLATION

SEQ ID NO: 46 - protein, 13 aa, Artificial Sequence
  Synthetic polypeptide
  MOD_RES (7)..(7): PHOSPHORYLATION
EGRSAGSIPGRRS

SEQ ID NO: 47 - protein, 11 aa, Artificial Sequence
  Synthetic polypeptide
  MOD_RES (6)..(6): PHOSPHORYLATION
IHRSASEPSLN

SEQ ID NO: 48 - protein, 13 aa, Artificial Sequence
  Synthetic polypeptide
  MOD_RES (8)..(8): PHOSPHORYLATION
SQRQRSTSTPNVH

SEQ ID NO: 49 - protein, 11 aa, Artificial Sequence
  Synthetic polypeptide
  MOD_RES (6)..(6): PHOSPHORYLATION
RRRAHTFSHPP

SEQ ID NO: 50 - protein, 11 aa, Artificial Sequence
  Synthetic polypeptide
  MOD_RES (6)..(6): PHOSPHORYLATION
EWRSVTLPRDL

SEQ ID NO: 51 - protein, 28 aa, Artificial Sequence
  Synthetic polypeptide
  MOD_RES (7)..(7): PHOSPHORYLATION
  MOD_RES (22)..(22): PHOSPHORYLATION
AILPRISVISTGPTLQARRRQSVLNLMT

SEQ ID NO: 52 - protein, 11 aa, Artificial Sequence
  Synthetic polypeptide
  MOD_RES (6)..(6): PHOSPHORYLATION
MRRASSYSSLN

SEQ ID NO: 53 - protein, 13 aa, Artificial Sequence
  Synthetic polypeptide
  MOD_RES (7)..(7): PHOSPHORYLATION
GVTRSASSPRLSS

SEQ ID NO: 54 - protein, 9 aa, Artificial Sequence
  Synthetic polypeptide
  MOD_RES (6)..(6): PHOSPHORYLATION
KRRRKSCQA

SEQ ID NO: 55 - protein, 6 aa, Artificial Sequence
  Synthetic polypeptide
  MISC_FEATURE (5)..(5): Phosphorylated Ser-373
KRRKSV

SEQ ID NO: 56 - protein, 10 aa, Artificial Sequence
  Synthetic polypeptide
GLLDALDLAS

SEQ ID NO: 57 - protein, 10 aa, Artificial Sequence
  Synthetic polypeptide
  MISC_FEATURE (4)..(4): Phosphorylated Ser-89
RSHSSPASLQ